SEQUENCE LISTING (879 sequences)

SEQ ID NO: 1 - protein, 20 aa, synthetic construct
GKPGSGKPGSGKPGSGKPGS

SEQ ID NO: 2 - protein, 5 aa, synthetic construct
GGGGS

SEQ ID NO: 3 - protein, 5 aa, synthetic construct
GSGGS

SEQ ID NO: 4 - protein, 5 aa, synthetic construct
GGGGS

SEQ ID NO: 5 - protein, 4 aa, synthetic construct
GGGS

SEQ ID NO: 6 - protein, 15 aa, synthetic construct
EPKSCGKPGSGKPGS

SEQ ID NO: 7 - protein, 383 aa, Homo sapiens
  source 1..383: note = sp|Q29983|MICA_HUMAN MHC class I polypeptide-related sequence A OS=Homo sapiens OX=9606; GN = MICA PE=1 SV=1
MGLGPVFLLLAGIFPFAPPGAAAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 8 - protein, 284 aa, Homo sapiens
  source 1..284: note = sp|Q29983|24-307
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHW

SEQ ID NO: 9 - protein, 366 aa, Macaca fascicularis
  source 1..366: note = UJY53392.1 MHC class I polypeptide-related sequence A [Macaca fascicularis]
MGPGRVLLFLASMFPFARPGAAAELHSLRYNVTVLSRDGSVQSEFLAEGHLDSQLFVRYDRETRRARPQGQWAEAVLGAKTWDTETGDLTENGKDLRRTLTHIEGQKGGLHSLQDIQNCEIYEDGSTGGSRHFYYDGERFLSLNLATQEWTVAQSSRAQTLAMNFWKEDTMKTNTHYHAMQADCLKKLRRYQKSRVAVRRTAPPMVNVTHSEASEGNITVTCRASGFYPRNIALTWRQDGVSLNHDAQRWGGILPDQNGTYQTWVATRIRQGEEQRFACYMEHSGNHSTHTVPSGKVLVFQSQWLDIPYVLGVAAAAVAAAAAIFVIILYVLCCKKKTSAAEGPELVSLRTLDQHPVGTGDHRDAT

SEQ ID NO: 10 - protein, 286 aa, Homo sapiens
  source 1..286: note = Human MICA*002, extracellular domain
AEPHSLRYNLTVLSGDGSVQSGFLAEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETEEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQ

SEQ ID NO: 11 - protein, 286 aa, Homo sapiens
  source 1..286: note = Human MICA*004, extracellular domain
AEPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNVETEEWTVPQSSRAQTLAMNVRNFLKEDAMKTKTHYHAMHADCLQELRRYLESSVVLRRRVPPMVNVTRSEASEGNITVTCRASSFYPRNITLTWRQDGVSLSHDTQQWGDVLPDGNGTYQTWVATRICQGEEQRFTCYMEHSGNHSTHPVPSGKVLVLQSHWQ

SEQ ID NO: 12 - protein, 286 aa, Homo sapiens
  source 1..286: note = Human MICA*008, extracellular domain
AEPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNLETEEWTVPQSSRAQTLAMNVRNFLKEDAMKTKTHYHAMHADCLQELRRYLESGVVLRRTVPPMVNVTRSEASEGNITVTCRASSFYPRNIILTWRQDGVSLSHDTQQWGDVLPDGNGTYQTWVATRICRGEEQRFTCYMEHSGNHSTHPVPSGKVLVLQSHWQ

SEQ ID NO: 13 - protein, 383 aa, Homo sapiens
  source 1..383: note = sp|Q29980|MICB_HUMAN MHC class I polypeptide-related sequence B OS=Homo sapiens OX=9606; GN = MICB PE=1 SV=2
MGLGRVLLFLAVAFPFAPPAAAAEPHSLRYNLMVLSQDGSVQSGFLAEGHLDGQPFLRYDRQKRRAKPQGQWAENVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDFPYVSAAMPCFVIIIILCVPCCKKKTSAAEGPELVSLQVLDQHPVGTGDHRDAAQLGFQPLMSATGSTGSTEGT

SEQ ID NO: 14 - protein, 287 aa, Homo sapiens
  source 1..287: note = sp|Q29980|23-309
AEPHSLRYNLMVLSQDGSVQSGFLAEGHLDGQPFLRYDRQKRRAKPQGQWAENVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTD

SEQ ID NO: 15 - protein, 384 aa, Macaca fascicularis
  source 1..384: note = UJY53393.1 MHC class I polypeptide-related sequence B [Macaca fascicularis]
MGLGRVLLFLGRVLLFLASIFSFTRPGAAAELHSLRYNVTVLSRDGSVQSGFLAEGHLDGQLFVRYDRETRRARPQGQWAEAVLGDQETEDLTENAQDLRRTLTHIEGQKGGLHSLQKIKICEIYEDGSTGGFWHFYYDGELFLSLNLETQKWTVAQSSRAQTLAMSFWKEDTMKTNTHYRAMRADCLKKLRRYQKSRVAVRRTVPPMVNVTHGEASEGNITVTCRASGFYPGNITLTWRQDGVSLSHDAQQWGDVLPDRNGTYYTWVATRIRQGEEQKFACYMEHSGNHSTHPVPSGKPPVLQSERLNLLYVPVAVAVVTAFIIIRVHCCKKKKTSAAEGPELVSLQTLDQHPVGTGDHRDATQLGSQPLMSAPGSTGSTEGA

SEQ ID NO: 16 - protein, 287 aa, Homo sapiens
  source 1..287: note = Human MICB*005, extracellular domain
AEPHSLRYNLMVLSQDGSVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTD

SEQ ID NO: 17 - protein, 304 aa, Homo sapiens
  source 1..304: note = sp|O76036|NCTR1_HUMAN Natural cytotoxicity triggering receptor 1 OS=Homo sapiens OX=9606 GN=NCR1 PE=1; SV = 1
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 18 - protein, 237 aa, Homo sapiens
  source 1..237: note = sp|O76036|NCTR1_HUMAN|22-258 Natural cytotoxicity triggering receptor 1 OS=Homo sapiens OX=9606 GN=NCR1 PE=1; SV = 1
QQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLR

SEQ ID NO: 19 - protein, 325 aa, Mus musculus
  source 1..325: note = sp|Q8C567|NCTR1_MOUSE Natural cytotoxicity triggering receptor 1 OS=Mus musculus OX=10090 GN=Ncr1; PE = 1 SV=2
MLPTLTALLCLGLCLSQRINTEKETLPKPIIWAKPSIMVTNGNSVNIWCQGAQSASEYQLYFEGSFFALERPKPSRSMNKVRFFISQMTSHTAGIYTCFYQSGELWSKSSNPLKLVVTGLYDTPNLWVYPRPEVTLGENVTFFCQLKTATSKFFLLKERGSNHIQNKYGNIQAEFPMGPVTRAHRGTYRCFGSYNDYAWSFPSEPVTLLITGGVENSSLAPTDPTSSLDYWEFDLSTNESGLQKDSAFWDHTTQNLIRIGLACIILITLVWLLTEDWLSKRKDHEEANRLTNWECRRRWRMQHYFEEEQRNAISMMELKATPGAL

SEQ ID NO: 20 - protein, 239 aa, Mus musculus
  source 1..239: note = sp|Q8C567|NCTR1_MOUSE|17-255 Natural cytotoxicity triggering receptor 1 OS=Mus musculus OX=10090 GN=Ncr1; PE = 1 SV=2
QRINTEKETLPKPIIWAKPSIMVTNGNSVNIWCQGAQSASEYQLYFEGSFFALERPKPSRSMNKVRFFISQMTSHTAGIYTCFYQSGELWSKSSNPLKLVVTGLYDTPNLWVYPRPEVTLGENVTFFCQLKTATSKFFLLKERGSNHIQNKYGNIQAEFPMGPVTRAHRGTYRCFGSYNDYAWSFPSEPVTLLITGGVENSSLAPTDPTSSLDYWEFDLSTNESGLQKDSAFWDHTTQN

SEQ ID NO: 21 - protein, 306 aa, Macaca fascicularis
  source 1..306: note = sp|Q95JB9|NCTR1_MACFA Natural cytotoxicity triggering receptor 1 OS=Macaca fascicularis OX=9541; GN = NCR1 PE=2 SV=1
MSSTLRALLCLGLCLSQRISAPKQTLPKPIIRAESTYMVPKEKQATLCCQGSYGAVEYQLHFEGSLFAVERPKPPERINGVKFHIPDMNSRKAGRYSCIYRVGELWSERSDLLDLVVTEMYDTPTLSVHPGPEVTSGEKVTFYCRLDTATSMFLLLKEGRSRDVQRSYGKVQAEFPMGPVTTAHRGSYRCFGSYNNYAWSFPSEPVKLLVTGDIENTSLAPTDPTFPDSWDTCLLTRETGLQKDLALWDHTAQNLLRMGLAFLVLVALVCLLVEDWLSRKRTREQASRASTWEGRRRLNKHKDSEE

SEQ ID NO: 22 - protein, 236 aa, Macaca fascicularis
  source 1..236: note = sp|Q95JB9|NCTR1_MACFA|22-257 Natural cytotoxicity triggering receptor 1 OS=Macaca fascicularis OX=9541; GN = NCR1 PE=2 SV=1
PKQTLPKPIIRAESTYMVPKEKQATLCCQGSYGAVEYQLHFEGSLFAVERPKPPERINGVKFHIPDMNSRKAGRYSCIYRVGELWSERSDLLDLVVTEMYDTPTLSVHPGPEVTSGEKVTFYCRLDTATSMFLLLKEGRSRDVQRSYGKVQAEFPMGPVTTAHRGSYRCFGSYNNYAWSFPSEPVKLLVTGDIENTSLAPTDPTFPDSWDTCLLTRETGLQKDLALWDHTAQNLLR

SEQ ID NO: 23 - protein, 15 aa, synthetic construct
  source 1..15: note = Gly-Ser 15
GGGGSGGGGSGGGGS

SEQ ID NO: 24 - protein, 18 aa, synthetic construct
  source 1..18: note = Whitlow linker
GSTSGSGKPGSGEGSTKG

SEQ ID NO: 25 - protein, 14 aa, synthetic construct
  source 1..14: note = 6paxA_1 (+A)
IRPRAIGGSKPRVA

SEQ ID NO: 26 - protein, 15 aa, synthetic construct
  source 1..15: note = +B
GKGGSGKGGSGKGGS

SEQ ID NO: 27 - protein, 15 aa, synthetic construct
  source 1..15: note = +C
GGKGSGGKGSGGKGS

SEQ ID NO: 28 - protein, 15 aa, synthetic construct
  source 1..15: note = +D
GGGKSGGGKSGGGKS

SEQ ID NO: 29 - protein, 15 aa, synthetic construct
  source 1..15: note = +E
GKGKSGKGKSGKGKS

SEQ ID NO: 30 - protein, 15 aa, synthetic construct
  source 1..15: note = +F
GGGKSGGKGSGKGGS

SEQ ID NO: 31 - protein, 15 aa, synthetic construct
  source 1..15: note = +G
GKPGSGKPGSGKPGS

SEQ ID NO: 32 - protein, 20 aa, synthetic construct
  source 1..20: note = +H
GKPGSGKPGSGKPGSGKPGS

SEQ ID NO: 33 - protein, 20 aa, synthetic construct
  source 1..20: note = +I
GKGKSGKGKSGKGKSGKGKS

SEQ ID NO: 34 - protein, 20 aa, synthetic construct
  source 1..20: note = Gly-Ser 20
GGGGSGGGGSGGGGSGGGGS

SEQ ID NO: 35 - protein, 14 aa, synthetic construct
  source 1..14: note = 3hsc_2 (-A)
STAGDTHLGGEDFD

SEQ ID NO: 36 - protein, 15 aa, synthetic construct
  source 1..15: note = -B
GEGGSGEGGSGEGGS

SEQ ID NO: 37 - protein, 15 aa, synthetic construct
  source 1..15: note = -C
GGEGSGGEGSGGEGS

SEQ ID NO: 38 - protein, 15 aa, synthetic construct
  source 1..15: note = -D
GGGESGGGESGGGES

SEQ ID NO: 39 - protein, 15 aa, synthetic construct
  source 1..15: note = -E
GEGESGEGESGEGES

SEQ ID NO: 40 - protein, 15 aa, synthetic construct
  source 1..15: note = -F
GGGESGGEGSGEGGS

SEQ ID NO: 41 - protein, 20 aa, synthetic construct
  source 1..20: note = -G
GEGESGEGESGEGESGEGES

SEQ ID NO: 42 - protein, 15 aa, synthetic construct
GGGGSGGGGSGGGGS

SEQ ID NO: 43 - protein, 20 aa, synthetic construct
GGGGSGGGGSGGGGSGGGGS

SEQ ID NO: 44 - protein, 18 aa, synthetic construct
GSTSGSGKPGSGEGSTKG

SEQ ID NO: 45 - protein, 19 aa, synthetic construct
PRGASKSGSASQTGSAPGS

SEQ ID NO: 46 - DNA, 19 bp, synthetic construct
  source 1..19: mol_type = other DNA
gtaaagagaaggaaagaag

SEQ ID NO: 47 - protein, 19 aa, synthetic construct
GTSGSSGSGSGGSGSGGGG

SEQ ID NO: 48 - protein, 20 aa, synthetic construct
GKPGSGKPGSGKPGSGKPGS

SEQ ID NO: 49 - protein, 5 aa, synthetic construct
  source 1..5: note = (GGGGS)1 or GGGGS
GGGGS

SEQ ID NO: 50 - protein, 10 aa, synthetic construct
  source 1..10: note = (GGGGS)2
GGGGSGGGGS

SEQ ID NO: 51 - protein, 15 aa, synthetic construct
  source 1..15: note = (GGGGS)3
GGGGSGGGGSGGGGS

SEQ ID NO: 52 - protein, 20 aa, synthetic construct
  source 1..20: note = (GGGGS)4
GGGGSGGGGSGGGGSGGGGS

SEQ ID NO: 53 - protein, 25 aa, synthetic construct
  source 1..25: note = (GGGGS)5
GGGGSGGGGSGGGGSGGGGSGGGGS

SEQ ID NO: 54 - protein, 30 aa, synthetic construct
  source 1..30: note = (GGGGS)6
GGGGSGGGGSGGGGSGGGGSGGGGSGGGGS

SEQ ID NO: 55 - protein, 35 aa, synthetic construct
  source 1..35: note = (GGGGS)7
GGGGSGGGGSGGGGSGGGGSGGGGSGGGGSGGGGS

SEQ ID NO: 56 - protein, 5 aa, synthetic construct
  source 1..5: note = (GGGGA)1 or GGGGA
GGGGA

SEQ ID NO: 57 - protein, 10 aa, synthetic construct
  source 1..10: note = (GGGGA)2
GGGGAGGGGA

SEQ ID NO: 58 - protein, 15 aa, synthetic construct
  source 1..15: note = (GGGGA)3
GGGGAGGGGAGGGGA

SEQ ID NO: 59 - protein, 20 aa, synthetic construct
  source 1..20: note = (GGGGA)4
GGGGAGGGGAGGGGAGGGGA

SEQ ID NO: 60 - protein, 25 aa, synthetic construct
  source 1..25: note = (GGGGA)5
GGGGAGGGGAGGGGAGGGGAGGGGA

SEQ ID NO: 61 - protein, 30 aa, synthetic construct
  source 1..30: note = (GGGGA)6
GGGGAGGGGAGGGGAGGGGAGGGGAGGGGA

SEQ ID NO: 62 - protein, 35 aa, synthetic construct
  source 1..35: note = (GGGGA)7
GGGGAGGGGAGGGGAGGGGAGGGGAGGGGAGGGGA

SEQ ID NO: 63 - protein, 30 aa, synthetic construct
  source 1..30: note = 30AA-linker
DPALVHQRPAPPGGGGSGGGGSGGGGSGGG

SEQ ID NO: 64 - protein, 5 aa, synthetic construct
  source 1..5: note = (GKPGS)1 or GKPGS
GKPGS

SEQ ID NO: 65 - protein, 25 aa, synthetic construct
  source 1..25: note = (GKPGS)5
GKPGSGKPGSGKPGSGKPGSGKPGS

SEQ ID NO: 66 - protein, 30 aa, synthetic construct
  source 1..30: note = (GKPGS)6
GKPGSGKPGSGKPGSGKPGSGKPGSGKPGS

SEQ ID NO: 67 - protein, 5 aa, synthetic construct
  source 1..5: note = (GGGES)1 or GGGES
GGGES

SEQ ID NO: 68 - protein, 15 aa, synthetic construct
  source 1..15: note = "full hinge"
EPKSCDKTHTCPPCP

SEQ ID NO: 69 - protein, 9 aa, synthetic construct
  source 1..9: note = "lower half hinge"
KTHTCPPCP

SEQ ID NO: 70 - protein, 15 aa, synthetic construct
  source 1..15: note = "full hinge C220S variant"
EPKSSDKTHTCPPCP

SEQ ID NO: 71 - protein, 19 aa, synthetic construct
  source 1..19: note = "flex lower half hinge"
GGGGSGGGGSKTHTCPPCP

SEQ ID NO: 72 - protein, 19 aa, synthetic construct
  source 1..19: note = "charged lower half hinge1"
GKPGSGKPGSKTHTCPPCP

SEQ ID NO: 73 - protein, 14 aa, synthetic construct
  source 1..14: note = "charged lower half hinge2"
GKPGSKTHTCPPCP

SEQ ID NO: 74 - protein, 5 aa, synthetic construct
  source 1..5: note = "upper half hinge"
EPKSC

SEQ ID NO: 75 - protein, 15 aa, synthetic construct
  source 1..15: note = "flex upper half hinge"
EPKSCGGGGSGGGGS

SEQ ID NO: 76 - protein, 15 aa, synthetic construct
  source 1..15: note = "charged upper half hinge1"
EPKSCGKPGSGKPGS

SEQ ID NO: 77 - protein, 10 aa, synthetic construct
  source 1..10: note = "charged upper half hinge2"
EPKSCGKPGS

SEQ ID NO: 78 - protein, 216 aa, synthetic construct
  source 1..216: note = FcKO Heterodimeric Fc Backbone 1 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 79 - protein, 216 aa, synthetic construct
  source 1..216: note = FcKO Heterodimeric Fc Backbone 1 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 216 aa, synthetic construct; identical to SEQ ID NO: 78
  source 1..216: note = FcKO Heterodimeric Fc Backbone 2 monomer 1 (-)

SEQ ID NO: 81 - protein, 216 aa, synthetic construct
  source 1..216: note = FcKO Heterodimeric Fc Backbone monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 216 aa, synthetic construct
  source 1..216: note = FcKO Heterodimeric Fc Backbone 3 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCEVSGFYPSDIAVEWESDGQPENNYKTTPPVLDSDGSFFLYSKLTVDKSRWEQGDVFSCSVMHEALHNHYTQKSLSLSPGK

SEQ ID NO: 83 - protein, 216 aa, synthetic construct; identical to SEQ ID NO: 81
  source 1..216: note = FcKO Heterodimeric Fc Backbone 3 monomer 2 (+)

SEQ ID NO: 84 - protein, 216 aa, synthetic construct
  source 1..216: note = FcKO Heterodimeric Fc Backbone 4 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTENEVSLTCLVKGFYPSDIAVEWESDGQPENNYKTTPPVLDSDGSFFLYSKLEVDKSRWEQGDVFSCSVMHEALHNHYTQKSLSLSPGK

SEQ ID NO: 85 - protein, 216 aa, synthetic construct
  source 1..216: note = FcKO Heterodimeric Fc Backbone 4 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 86 - protein, 216 aa, synthetic construct
  source 1..216: note = FcKO Heterodimeric Fc Backbone 5 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 87 - protein, 216 aa, synthetic construct
  source 1..216: note = FcKO Heterodimeric Fc Backbone 5 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 216 aa, synthetic construct
  source 1..216: note = FcKO Heterodimeric Fc Backbone 6 monomer 1 (-)
APPVAGPSVFLFPPKPKDTLMISRTPEVTCVVVDVKHEDPEVKFNWYVDGVEVHNAKTKPREEEYASTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 89 - protein, 216 aa, synthetic construct
  source 1..216: note = FcKO Heterodimeric Fc Backbone 6 monomer 2 (+)
APPVAGPSVFLFPPKPKDTLMISRTPEVTCVVVDVKHEDPEVKFNWYVDGVEVHNAKTKPREEQYASTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 216 aa, synthetic construct
  source 1..216: note = FcKO Heterodimeric Fc Backbone 7 monomer 1 (-)
APPVAGPSVFLFPPKPKDTLMISRTPEVTCVVVDVKHEDPEVKFNWYVDGVEVHNAKTKPREEEYSSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 91 - protein, 216 aa, synthetic construct
  source 1..216: note = FcKO Heterodimeric Fc Backbone 7 monomer 2 (+)
APPVAGPSVFLFPPKPKDTLMISRTPEVTCVVVDVKHEDPEVKFNWYVDGVEVHNAKTKPREEQYSSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 217 aa, synthetic construct
  source 1..217: note = FcKO Heterodimeric Fc Backbone 8 monomer 1 (-)
APEFLGGPSVFLFPPKPKDTLMISRTPEVTCVVVDVSQEDPEVQFNWYVDGVEVHNAKTKPREEEFNSTYRVVSVLTVLHQDWLNGKEYKCKVSNKGLPSSIEKTISKAKGQPREPQVYTLPPSQEEMTKNQVSLTCDVSGFYPSDIAVEWESDGQPENNYKTTPPVLDSDGSFFLYSRLTVDKSRWEEGDVFSCSVMHEALHNHYTQKSLSLSLGK

SEQ ID NO: 93 - protein, 217 aa, synthetic construct
  source 1..217: note = FcKO Heterodimeric Fc Backbone 8 monomer 2 (+)
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEQMTKNQVKLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 216 aa, synthetic construct
  source 1..216: note = FcKO Heterodimeric Fc Backbone 9 monomer 1 (-)
APPVAGPSVFLFPPKPKDTLMISRTPEVTCVVVDVSHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 95 - protein, 216 aa, synthetic construct
  source 1..216: note = FcKO Heterodimeric Fc Backbone 9 monomer 2 (+)
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 96 - protein, 216 aa, synthetic construct
  source 1..216: note = FcKO Heterodimeric Fc Backbone 10 monomer 1 (-)
APPVAGPSVFLFPPKPKDTLMISRTPEVTCVVVDVKHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 97 - protein, 216 aa, synthetic construct
  source 1..216: note = FcKO Heterodimeric Fc Backbone 10 monomer 2 (+)
APPVAGPSVFLFPPKPKDTLMISRTPEVTCVVVDVKHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 98 - protein, 216 aa, synthetic construct
  source 1..216: note = FcKO Heterodimeric Fc Backbone 11 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 99 - protein, 216 aa, synthetic construct
  source 1..216: note = FcKO Heterodimeric Fc Backbone 11 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 100 - protein, 216 aa, synthetic construct
  source 1..216: note = FcKO Heterodimeric Fc Backbone 12 monomer 1 (-)
APPVAGPSVFLFPPKPKDTLMISRTPEVTCVVVDVKHEDPEVKFKWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 216 aa, synthetic construct; identical to SEQ ID NO: 78
  source 1..216: note = FcKO Heterodimeric Fc Backbone 12 monomer 2 (+)

SEQ ID NO: 102 - protein, 217 aa, synthetic construct
  source 1..217: note = FcKO Heterodimeric Fc Backbone 13 monomer 1
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 217 aa, synthetic construct
  source 1..217: note = FcKO Heterodimeric Fc Backbone 13 monomer 2
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 104 - protein, 217 aa, synthetic construct
  source 1..217: note = FcKO Heterodimeric Fc Backbone 14 monomer 1
APEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 217 aa, synthetic construct
  source 1..217: note = FcKO Heterodimeric Fc Backbone 14 monomer 2
APEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 106 - protein, 216 aa, synthetic construct
  source 1..216: note = FcKO Heterodimeric Fc Backbone 15 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 107 - protein, 216 aa, synthetic construct
  source 1..216: note = FcKO Heterodimeric Fc Backbone 15 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 108 - protein, 216 aa, synthetic construct
  source 1..216: note = FcKO Heterodimeric Fc Backbone 16 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 109 - protein, 216 aa, synthetic construct
  source 1..216: note = FcKO Heterodimeric Fc Backbone 16 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 110 - protein, 216 aa, synthetic construct
  source 1..216: note = FcKO Heterodimeric Fc Backbone 17 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 111 - protein, 216 aa, synthetic construct
  source 1..216: note = FcKO Heterodimeric Fc Backbone 17 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 112 - protein, 217 aa, synthetic construct
  source 1..217: note = Heterodimeric Fc Backbone 1 monomer 1 (-)
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 113 - protein, 217 aa, synthetic construct
  source 1..217: note = Heterodimeric Fc Backbone 1 monomer 2 (+)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 217 aa, synthetic construct; identical to SEQ ID NO: 112
  source 1..217: note = Heterodimeric Fc Backbone 2 monomer 1 (-)

SEQ ID NO: 115 - protein, 217 aa, synthetic construct
  source 1..217: note = Heterodimeric Fc Backbone monomer 2 (+)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 217 aa, synthetic construct
  source 1..217: note = Heterodimeric Fc Backbone 3 monomer 1 (-)
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCEVSGFYPSDIAVEWESDGQPENNYKTTPPVLDSDGSFFLYSKLTVDKSRWEQGDVFSCSVMHEALHNHYTQKSLSLSPGK

SEQ ID NO: 117 - protein, 217 aa, synthetic construct; identical to SEQ ID NO: 115
  source 1..217: note = Heterodimeric Fc Backbone 3 monomer 2 (+)

SEQ ID NO: 118 - protein, 217 aa, synthetic construct
  source 1..217: note = Heterodimeric Fc Backbone 4 monomer 1 (-)
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTENEVSLTCLVKGFYPSDIAVEWESDGQPENNYKTTPPVLDSDGSFFLYSKLEVDKSRWEQGDVFSCSVMHEALHNHYTQKSLSLSPGK

SEQ ID NO: 119 - protein, 217 aa, synthetic construct
  source 1..217: note = Heterodimeric Fc Backbone 4 monomer 2 (+)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 120 - protein, 217 aa, synthetic construct
  source 1..217: note = Heterodimeric Fc Backbone 5 monomer 1 (-)
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 121 - protein, 217 aa, synthetic construct
  source 1..217: note = Heterodimeric Fc Backbone 5 monomer 2 (+)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 217 aa, synthetic construct
  source 1..217: note = Heterodimeric Fc Backbone 6 monomer 1 (-)
APELLGGPSVFLFPPKPKDTLMISRTPEVTCVVVDVSHEDPEVKFNWYVDGVEVHNAKTKPREEEYASTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 123 - protein, 217 aa, synthetic construct
  source 1..217: note = Heterodimeric Fc Backbone 6 monomer 2 (+)
APELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 217 aa, synthetic construct
  source 1..217: note = Heterodimeric Fc Backbone 7 monomer 1 (-)
APELLGGPSVFLFPPKPKDTLMISRTPEVTCVVVDVSHEDPEVKFNWYVDGVEVHNAKTKPREEEYSSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 125 - protein, 217 aa, synthetic construct
  source 1..217: note = Heterodimeric Fc Backbone 7 monomer 2 (+)
APELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 217 aa, synthetic construct
  source 1..217: note = Heterodimeric Fc Backbone 8 monomer 1 (-)
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 127 - protein, 217 aa, synthetic construct
  source 1..217: note = Heterodimeric Fc Backbone 8 monomer 2 (+)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 128 - protein, 217 aa, synthetic construct
  source 1..217: note = Heterodimeric Fc Backbone 9 monomer 1
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 129 - protein, 217 aa, synthetic construct
  source 1..217: note = Heterodimeric Fc Backbone 9 monomer 2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 130 - protein, 217 aa, synthetic construct
  source 1..217: note = Heterodimeric Fc Backbone 10 monomer 1
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 131 - protein, 217 aa, synthetic construct
  source 1..217: note = Heterodimeric Fc Backbone 10 monomer 2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 132 - protein, 217 aa, synthetic construct
  source 1..217: note = Heterodimeric Fc Backbone 11 monomer 1
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 133 - protein, 217 aa, synthetic construct
  source 1..217: note = Heterodimeric Fc Backbone 11 monomer 2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 134 - protein, 217 aa, synthetic construct
  source 1..217: note = S239D/I332E : S239D/I332E_ADCC-enhanced Heterodimeric Backbone 1 monomer 1 (-)
APELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 135 - protein, 217 aa, synthetic construct
  source 1..217: note = S239D/I332E : S239D/I332E_ADCC-enhanced Heterodimeric Backbone 1 monomer 2 (+)
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 217 aa, synthetic construct
  source 1..217: note = S239D : S239D_ADCC-enhanced Heterodimeric Backbone 2 monomer 1 (-)
APELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 137 - protein, 217 aa, synthetic construct
  source 1..217: note = S239D : S239D_ADCC-enhanced Heterodimeric Backbone 2 monomer 2 (+)
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 217 aa, synthetic construct
  source 1..217: note = I332E : I332E_ADCC-enhanced Heterodimeric Backbone 3 monomer 1 (-)
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 139 - protein, 217 aa, synthetic construct
  source 1..217: note = I332E : I332E_ADCC-enhanced Heterodimeric Backbone 3 monomer 2 (+)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 217 aa, synthetic construct; identical to SEQ ID NO: 112
  source 1..217: note = WT : S239D/I332E_ADCC-enhanced Heterodimeric Backbone 4 monomer 1 (-)

SEQ ID NO: 141 - protein, 217 aa, synthetic construct; identical to SEQ ID NO: 135
  source 1..217: note = WT : S239D/I332E_ADCC-enhanced Heterodimeric Backbone 4 monomer 2 (+)

SEQ ID NO: 142 - protein, 217 aa, synthetic construct; identical to SEQ ID NO: 112
  source 1..217: note = WT : S239D_ADCC-enhanced Heterodimeric Backbone 5 monomer 1 (-)

SEQ ID NO: 143 - protein, 217 aa, synthetic construct; identical to SEQ ID NO: 137
  source 1..217: note = WT : S239D_ADCC-enhanced Heterodimeric Backbone 5 monomer 2 (+)

SEQ ID NO: 144 - protein, 217 aa, synthetic construct; identical to SEQ ID NO: 112
  source 1..217: note = WT : I332E_ADCC-enhanced Heterodimeric Backbone 6 monomer 1 (-)

SEQ ID NO: 145 - protein, 217 aa, synthetic construct; identical to SEQ ID NO: 139
  source 1..217: note = WT : I332E_ADCC-enhanced Heterodimeric Backbone 6 monomer 2 (+)

SEQ ID NO: 146 - protein, 217 aa, synthetic construct; identical to SEQ ID NO: 134
  source 1..217: note = S239D/I332E : WT_ADCC-enhanced Heterodimeric Backbone 7 monomer 1 (-)

SEQ ID NO: 147 - protein, 217 aa, synthetic construct; identical to SEQ ID NO: 113
  source 1..217: note = S239D/I332E : WT_ADCC-enhanced Heterodimeric Backbone 7 monomer 2 (+)

SEQ ID NO: 148 - protein, 217 aa, synthetic construct; identical to SEQ ID NO: 136
  source 1..217: note = S239D : WT_ADCC-enhanced Heterodimeric Backbone 8 monomer 1 (-)

SEQ ID NO: 149 - protein, 217 aa, synthetic construct; identical to SEQ ID NO: 113
  source 1..217: note = S239D : WT_ADCC-enhanced Heterodimeric Backbone 8 monomer 2 (+)

SEQ ID NO: 150 - protein, 217 aa, synthetic construct; identical to SEQ ID NO: 138
  source 1..217: note = I332E : WT_ADCC-enhanced Heterodimeric Backbone 9 monomer 1 (-)

SEQ ID NO: 151 - protein, 217 aa, synthetic construct; identical to SEQ ID NO: 113
  source 1..217: note = I332E : WT_ADCC-enhanced Heterodimeric Backbone 9 monomer 2 (+)

SEQ ID NO: 152 - protein, 217 aa, synthetic construct; identical to SEQ ID NO: 134
  source 1..217: note = S239D/I332E : S239D_ADCC-enhanced Heterodimeric Backbone 10 monomer 1 (-)

SEQ ID NO: 153 - protein, 217 aa, synthetic construct; identical to SEQ ID NO: 137
  source 1..217: note = S239D/I332E : S239D_ADCC-enhanced Heterodimeric Backbone 10 monomer 2 (+)

SEQ ID NO: 154 - protein, 217 aa, synthetic construct; identical to SEQ ID NO: 134
  source 1..217: note = S239D/I332E : I332E_ADCC-enhanced Heterodimeric Backbone 11 monomer 1 (-)

SEQ ID NO: 155 - protein, 217 aa, synthetic construct; identical to SEQ ID NO: 139
  source 1..217: note = S239D/I332E : I332E_ADCC-enhanced Heterodimeric Backbone 11 monomer 2 (+)

SEQ ID NO: 156 - protein, 217 aa, synthetic construct; identical to SEQ ID NO: 136
  source 1..217: note = S239D : S239D/I332E_ADCC-enhanced Heterodimeric Backbone 12 monomer 1 (-)

SEQ ID NO: 157 - protein, 217 aa, synthetic construct; identical to SEQ ID NO: 135
  source 1..217: note = S239D : S239D/I332E_ADCC-enhanced Heterodimeric Backbone 12 monomer 2 (+)

SEQ ID NO: 158 - protein, 217 aa, synthetic construct; identical to SEQ ID NO: 138
  source 1..217: note = I332E : S239D/I332E_ADCC-enhanced Heterodimeric Backbone 13 monomer 1 (-)

SEQ ID NO: 159 - protein, 217 aa, synthetic construct; identical to SEQ ID NO: 135
  source 1..217: note = I332E : S239D/I332E_ADCC-enhanced Heterodimeric Backbone 13 monomer 2 (+)

SEQ ID NO: 160 - protein, 217 aa, synthetic construct; identical to SEQ ID NO: 136
  source 1..217: note = S239D : I332E_ADCC-enhanced Heterodimeric Backbone 14 monomer 1 (-)

SEQ ID NO: 161 - protein, 217 aa, synthetic construct; identical to SEQ ID NO: 139
  source 1..217: note = S239D : I332E_ADCC-enhanced Heterodimeric Backbone 14 monomer 2 (+)

SEQ ID NO: 162 - protein, 217 aa, synthetic construct; identical to SEQ ID NO: 138
  source 1..217: note = I332E : S239D_ADCC-enhanced Heterodimeric Backbone 15 monomer 1 (-)

SEQ ID NO: 163 - protein, 217 aa, synthetic construct; identical to SEQ ID NO: 137
  source 1..217: note = I332E : S239D_ADCC-enhanced Heterodimeric Backbone 15 monomer 2 (+)

SEQ ID NO: 164 - protein, 217 aa, synthetic construct
  source 1..217: note = S239D/I332E : S239D/I332E_ADCC-enhanced Heterodimeric Backbone 1 w/ Xtend monomer 1 (-)
APELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 165 - protein, 217 aa, synthetic construct
  source 1..217: note = S239D/I332E : S239D/I332E_ADCC-enhanced Heterodimeric Backbone 1 w/ Xtend monomer 2 (+)
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 166 - protein, 217 aa, synthetic construct
  source 1..217: note = S239D : S239D_ADCC-enhanced Heterodimeric Backbone 2 w/ Xtend monomer 1 (-)
APELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 167 - protein, 217 aa, synthetic construct
  source 1..217: note = S239D : S239D_ADCC-enhanced Heterodimeric Backbone 2 w/ Xtend monomer 2 (+)
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 168 - protein, 217 aa, synthetic construct
  source 1..217: note = I332E : I332E_ADCC-enhanced Heterodimeric Backbone 3 w/ Xtend monomer 1 (-)
APELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 169 - protein, 217 aa, synthetic construct
  source 1..217: note = I332E : I332E_ADCC-enhanced Heterodimeric Backbone 3 w/ Xtend monomer 2 (+)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 170 - protein, 217 aa, synthetic construct; identical to SEQ ID NO: 126
  source 1..217: note = WT : S239D/I332E_ADCC-enhanced Heterodimeric Backbone 4 w/ Xtend monomer 1 (-)

SEQ ID NO: 171 - protein, 217 aa, synthetic construct; identical to SEQ ID NO: 165
  source 1..217: note = WT : S239D/I332E_ADCC-enhanced Heterodimeric Backbone 4 w/ Xtend monomer 2 (+)

SEQ ID NO: 172 - protein, 217 aa, synthetic construct; identical to SEQ ID NO: 126
  source 1..217: note = WT : S239D_ADCC-enhanced Heterodimeric Backbone 5 w/ Xtend monomer 1 (-)

SEQ ID NO: 173 - protein, 217 aa, synthetic construct; identical to SEQ ID NO: 167
  source 1..217: note = WT : S239D_ADCC-enhanced Heterodimeric Backbone 5 w/ Xtend monomer 2 (+)

SEQ ID NO: 174 - protein, 217 aa, synthetic construct; identical to SEQ ID NO: 126
  source 1..217: note = WT : I332E_ADCC-enhanced Heterodimeric Backbone 6 w/ Xtend monomer 1 (-)

SEQ ID NO: 175 - protein, 217 aa, synthetic construct; identical to SEQ ID NO: 169
  source 1..217: note = WT : I332E_ADCC-enhanced Heterodimeric Backbone 6 w/ Xtend monomer 2 (+)

SEQ ID NO: 176 - protein, 217 aa, synthetic construct; identical to SEQ ID NO: 164
  source 1..217: note = S239D/I332E : WT_ADCC-enhanced Heterodimeric Backbone 7 w/ Xtend monomer 1 (-)

SEQ ID NO: 177 - protein, 217 aa, synthetic construct; identical to SEQ ID NO: 127
  source 1..217: note = S239D/I332E : WT_ADCC-enhanced Heterodimeric Backbone 7 w/ Xtend monomer 2 (+)

SEQ ID NO: 178 - protein, 217 aa, synthetic construct; identical to SEQ ID NO: 166
  source 1..217: note = S239D : WT_ADCC-enhanced Heterodimeric Backbone 8 w/ Xtend monomer 1 (-)

SEQ ID NO: 179 - protein, 217 aa, synthetic construct; identical to SEQ ID NO: 127
  source 1..217: note = S239D : WT_ADCC-enhanced Heterodimeric Backbone 8 w/ Xtend monomer 2 (+)

SEQ ID NO: 180 - protein, 217 aa, synthetic construct; identical to SEQ ID NO: 168
  source 1..217: note = I332E : WT_ADCC-enhanced Heterodimeric Backbone 9 w/ Xtend monomer 1 (-)

SEQ ID NO: 181 - protein, 217 aa, synthetic construct; identical to SEQ ID NO: 127
  source 1..217: note = I332E : WT_ADCC-enhanced Heterodimeric Backbone 9 w/ Xtend monomer 2 (+)

SEQ ID NO: 182 - protein, 217 aa, synthetic construct; identical to SEQ ID NO: 164
  source 1..217: note = S239D/I332E : S239D_ADCC-enhanced Heterodimeric Backbone 10 w/ Xtend monomer 1 (-)

SEQ ID NO: 183 - protein, 217 aa, synthetic construct; identical to SEQ ID NO: 167
  source 1..217: note = S239D/I332E : S239D_ADCC-enhanced Heterodimeric Backbone 10 w/ Xtend monomer 2 (+)

SEQ ID NO: 184 - protein, 217 aa, synthetic construct; identical to SEQ ID NO: 164
  source 1..217: note = S239D/I332E : I332E_ADCC-enhanced Heterodimeric Backbone 11 w/ Xtend monomer 1 (-)

SEQ ID NO: 185 - protein, 217 aa, synthetic construct; identical to SEQ ID NO: 169
  source 1..217: note = S239D/I332E : I332E_ADCC-enhanced Heterodimeric Backbone 11 w/ Xtend monomer 2 (+)

SEQ ID NO: 186 - protein, 217 aa, synthetic construct; identical to SEQ ID NO: 166
  source 1..217: note = S239D : S239D/I332E_ADCC-enhanced Heterodimeric Backbone 12 w/ Xtend monomer 1 (-)

SEQ ID NO: 187 - protein, 217 aa, synthetic construct; identical to SEQ ID NO: 165
  source 1..217: note = S239D : S239D/I332E_ADCC-enhanced Heterodimeric Backbone 12 w/ Xtend monomer 2 (+)

SEQ ID NO: 188 - protein, 217 aa, synthetic construct; identical to SEQ ID NO: 168
  source 1..217: note = I332E : S239D/I332E_ADCC-enhanced Heterodimeric Backbone 13 w/ Xtend monomer 1 (-)

SEQ ID NO: 189 - protein, 217 aa, synthetic construct; identical to SEQ ID NO: 165
  source 1..217: note = I332E : S239D/I332E_ADCC-enhanced Heterodimeric Backbone 13 w/ Xtend monomer 2 (+)

SEQ ID NO: 190 - protein, 217 aa, synthetic construct; identical to SEQ ID NO: 166
  source 1..217: note = S239D : I332E_ADCC-enhanced Heterodimeric Backbone 14 w/ Xtend monomer 1 (-)

SEQ ID NO: 191 - protein, 217 aa, synthetic construct; identical to SEQ ID NO: 169
  source 1..217: note = S239D : I332E_ADCC-enhanced Heterodimeric Backbone 14 w/ Xtend monomer 2 (+)

SEQ ID NO: 192 - protein, 217 aa, synthetic construct; identical to SEQ ID NO: 168
  source 1..217: note = I332E : S239D_ADCC-enhanced Heterodimeric Backbone 15 w/ Xtend monomer 1 (-)

SEQ ID NO: 193 - protein, 217 aa, synthetic construct; identical to SEQ ID NO: 167
  source 1..217: note = I332E : S239D_ADCC-enhanced Heterodimeric Backbone 15 w/ Xtend monomer 2 (+)

SEQ ID NO: 194 - protein, 216 aa, synthetic construct; identical to SEQ ID NO: 78
  source 1..216: note = 2 + 1 mAb-scFv Heterodimeric Fc Backbone FcKO monomer 1 (-)

SEQ ID NO: 195 - protein, 215 aa, synthetic construct
  source 1..215: note = 2 + 1 mAb-scFv Heterodimeric Fc Backbone FcKO monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 196 - protein, 217 aa, synthetic construct; identical to SEQ ID NO: 112
  source 1..217: note = 2 + 1 mAb-scFv Heterodimeric Fc Backbone (WT effector)_Heterodimeric Fc Backbone 1 monomer 1 (-)

SEQ ID NO: 197 - protein, 216 aa, synthetic construct
  source 1..216: note = 2 + 1 mAb-scFv Heterodimeric Fc Backbone (WT effector)_Heterodimeric Fc Backbone 1 monomer 2 (+)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 198 - protein, 216 aa, synthetic construct
  source 1..216: note = S239D/I332E : S239D/I332E_ADCC-enhanced Heterodimeric Backbone 1 monomer 2 (+)
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 199 - protein, 216 aa, synthetic construct
  source 1..216: note = S239D : S239D_ADCC-enhanced Heterodimeric Backbone 2 monomer 2 (+)
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 200 - protein, 216 aa, synthetic construct
  source 1..216: note = I332E : I332E_ADCC-enhanced Heterodimeric Backbone 3 monomer 2 (+)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 201 - protein, 216 aa, synthetic construct; identical to SEQ ID NO: 198
  source 1..216: note = WT : S239D/I332E_ADCC-enhanced Heterodimeric Backbone 4 monomer 2 (+)

SEQ ID NO: 202 - protein, 216 aa, synthetic construct; identical to SEQ ID NO: 199
  source 1..216: note = WT : S239D_ADCC-enhanced Heterodimeric Backbone 5 monomer 2 (+)

SEQ ID NO: 203 - protein, 216 aa, synthetic construct; identical to SEQ ID NO: 200
  source 1..216: note = WT : I332E_ADCC-enhanced Heterodimeric Backbone 6 monomer 2 (+)

SEQ ID NO: 204 - protein, 216 aa, synthetic construct; identical to SEQ ID NO: 197
  source 1..216: note = S239D/I332E : WT_ADCC-enhanced Heterodimeric Backbone 7 monomer 2 (+)

SEQ ID NO: 205 - protein, 216 aa, synthetic construct; identical to SEQ ID NO: 197
  source 1..216: note = S239D : WT_ADCC-enhanced Heterodimeric Backbone 8 monomer 2 (+)

SEQ ID NO: 206 - protein, 216 aa, synthetic construct; identical to SEQ ID NO: 197
  source 1..216: note = I332E : WT_ADCC-enhanced Heterodimeric Backbone 9 monomer 2 (+)

SEQ ID NO: 207 - protein, 216 aa, synthetic construct; identical to SEQ ID NO: 199
  source 1..216: note = S239D/I332E : S239D_ADCC-enhanced Heterodimeric Backbone 10 monomer 2 (+)

SEQ ID NO: 208 - protein, 216 aa, synthetic construct; identical to SEQ ID NO: 200
  source 1..216: note = S239D/I332E : I332E_ADCC-enhanced Heterodimeric Backbone 11 monomer 2 (+)

SEQ ID NO: 209 - protein, 216 aa, synthetic construct; identical to SEQ ID NO: 198
  source 1..216: note = S239D : S239D/I332E_ADCC-enhanced Heterodimeric Backbone 12 monomer 2 (+)

SEQ ID NO: 210 - protein, 216 aa, synthetic construct; identical to SEQ ID NO: 198
  source 1..216: note = I332E : S239D/I332E_ADCC-enhanced Heterodimeric Backbone 13 monomer 2 (+)

SEQ ID NO: 211 - protein, 216 aa, synthetic construct; identical to SEQ ID NO: 200
  source 1..216: note = S239D : I332E_ADCC-enhanced Heterodimeric Backbone 14 monomer 2 (+)

SEQ ID NO: 212 - protein, 216 aa, synthetic construct; identical to SEQ ID NO: 199
  source 1..216: note = I332E : S239D_ADCC-enhanced Heterodimeric Backbone 15 monomer 2 (+)

SEQ ID NO: 213 - protein, 216 aa, synthetic construct
  source 1..216: note = S239D/I332E : S239D/I332E_ADCC-enhanced Heterodimeric Backbone 1 w/ Xtend monomer 2 (+)
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 214 - protein, 216 aa, synthetic construct
  source 1..216: note = S239D : S239D_ADCC-enhanced Heterodimeric Backbone 2 w/ Xtend monomer 2 (+)
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 215 - protein, 216 aa, synthetic construct
  source 1..216: note = I332E : I332E_ADCC-enhanced Heterodimeric Backbone 3 w/ Xtend monomer 2 (+)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 216 - protein, 216 aa, synthetic construct; identical to SEQ ID NO: 213
  source 1..216: note = WT : S239D/I332E_ADCC-enhanced Heterodimeric Backbone 4 w/ Xtend monomer 2 (+)

SEQ ID NO: 217 - protein, 216 aa, synthetic construct; identical to SEQ ID NO: 214
  source 1..216: note = WT : S239D_ADCC-enhanced Heterodimeric Backbone 5 w/ Xtend monomer 2 (+)

SEQ ID NO: 218 - protein, 216 aa, synthetic construct; identical to SEQ ID NO: 215
  source 1..216: note = WT : I332E_ADCC-enhanced Heterodimeric Backbone 6 w/ Xtend monomer 2 (+)

SEQ ID NO: 219 - protein, 216 aa, synthetic construct
  source 1..216: note = S239D/I332E : WT_ADCC-enhanced Heterodimeric Backbone 7 w/ Xtend monomer 2 (+)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 220 - protein, 216 aa, synthetic construct; identical to SEQ ID NO: 219
  source 1..216: note = S239D : WT_ADCC-enhanced Heterodimeric Backbone 8 w/ Xtend monomer 2 (+)

SEQ ID NO: 221 - protein, 216 aa, synthetic construct; identical to SEQ ID NO: 219
  source 1..216: note = I332E : WT_ADCC-enhanced Heterodimeric Backbone 9 w/ Xtend monomer 2 (+)

SEQ ID NO: 222 - protein, 216 aa, synthetic construct; identical to SEQ ID NO: 214
  source 1..216: note = S239D/I332E : S239D_ADCC-enhanced Heterodimeric Backbone 10 w/ Xtend monomer 2 (+)

SEQ ID NO: 223 - protein, 216 aa, synthetic construct; identical to SEQ ID NO: 215
  source 1..216: note = S239D/I332E : I332E_ADCC-enhanced Heterodimeric Backbone 11 w/ Xtend monomer 2 (+)

SEQ ID NO: 224 - protein, 216 aa, synthetic construct; identical to SEQ ID NO: 213
  source 1..216: note = S239D : S239D/I332E_ADCC-enhanced Heterodimeric Backbone 12 w/ Xtend monomer 2 (+)

SEQ ID NO: 225 - protein, 216 aa, synthetic construct; identical to SEQ ID NO: 213
  source 1..216: note = I332E : S239D/I332E_ADCC-enhanced Heterodimeric Backbone 13 w/ Xtend monomer 2 (+)

SEQ ID NO: 226 - protein, 216 aa, synthetic construct; identical to SEQ ID NO: 215
  source 1..216: note = S239D : I332E_ADCC-enhanced Heterodimeric Backbone 14 w/ Xtend monomer 2 (+)

SEQ ID NO: 227 - protein, 216 aa, synthetic construct; identical to SEQ ID NO: 214
  source 1..216: note = I332E : S239D_ADCC-enhanced Heterodimeric Backbone 15 w/ Xtend monomer 2 (+)

SEQ ID NO: 228 - protein, 98 aa, synthetic construct
  source 1..98: note = IgG1 CH1(+)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 229 - protein, 98 aa, synthetic construct
  source 1..98: note = IgG1 CH1(-)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKV

SEQ ID NO: 230 - protein, 98 aa, synthetic construct
  source 1..98: note = IgG2 CH1(+)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 231 - protein, 98 aa, synthetic construct
  source 1..98: note = IgG2 CH1(-)
ASTKGPSVFPLAPCSRSTSESTAALGCLVKDYFPEPVTVSWNSGALTSGVHTFPAVLQSSGLYSLSSVVTVPSSNFGTQTYTCNVDHKPSDTKVDKTV

SEQ ID NO: 232 - protein, 98 aa, synthetic construct
  source 1..98: note = IgG4 CH1(+)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 233 - protein, 98 aa, synthetic construct
  source 1..98: note = IgG4 CH1(-)
ASTKGPSVFPLAPCSRSTSESTAALGCLVKDYFPEPVTVSWNSGALTSGVHTFPAVLQSSGLYSLSSVVTVPSSSLGTKTYTCNVDHKPSDTKVDKRV

SEQ ID NO: 234 - protein, 15 aa, synthetic construct
  source 1..15: note = IgG1 hinge
EPKSCDKTHTCPPCP

SEQ ID NO: 235 - protein, 12 aa, synthetic construct
  source 1..12: note = IgG2 hinge
ERKCCVECPPCP

SEQ ID NO: 236 - protein, 12 aa, synthetic construct
  source 1..12: note = IgG4 hinge
ESKYGPPCPSCP

SEQ ID NO: 237 - protein, 107 aa, synthetic construct [sequence omitted]

SEQ ID NO: 238 - protein, 106 aa, synthetic construct
  source 1..106: note = Light Chain Constant Domain - Lambda
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 239 - protein, 118 aa, synthetic construct
  source 1..118: note = D94837_1E11_1 [MICA/B]_H0L0 Variable heavy (vh) domain
EIQLQQSGPELVKPGASVKVSCKASGYAFTSYNMYWVKQSHGKSLEWIGYIDPYNGGTIYNQKFKGKATLTVDKSSSTAYMLLNSLTSEDSAVYYCARVGDAYPFAYWGQGTLVTVSA

SEQ ID NO: 240 - protein, 10 aa, synthetic construct
  source 1..10: note = D94837_1E11_1 [MICA/B]_H0L0 vhCDR1
GYAFTSYNMY

SEQ ID NO: 241 - protein, 17 aa, synthetic construct
  source 1..17: note = D94837_1E11_1 [MICA/B]_H0L0 vhCDR2
YIDPYNGGTIYNQKFKG

SEQ ID NO: 242 - protein, 9 aa, synthetic construct
  source 1..9: note = D94837_1E11_1 [MICA/B]_H0L0 vhCDR3
VGDAYPFAY

SEQ ID NO: 243 - protein, 111 aa, synthetic construct
  source 1..111: note = D94837_1E11_1 [MICA/B]_H0L0 Variable light (vl) domain
DIVLTQSPASLAVSLGQRATISCRASESVDNYGISFMNWFQQKAGQPPKLLIYAASNQGSGVPARFSGSGSGTDFSLNIHPMEEDDTAMYFCQQRKEVPFTFGSGTKLEIK

SEQ ID NO: 244 - protein, 15 aa, synthetic construct
  source 1..15: note = D94837_1E11_1 [MICA/B]_H0L0 vlCDR1
RASESVDNYGISFMN

SEQ ID NO: 245 - protein, 7 aa, synthetic construct
  source 1..7: note = D94837_1E11_1 [MICA/B]_H0L0 vlCDR2
AASNQGS

SEQ ID NO: 246 - protein, 9 aa, synthetic construct
  source 1..9: note = D94837_1E11_1 [MICA/B]_H0L0 vlCDR3
QQRKEVPFT

SEQ ID NO: 247 - protein, 118 aa, synthetic construct
  source 1..118: note = D94837_1E11_1 [MICA/B]_H1L1 Variable heavy (vh) domain
QIQLVQSGAEVKKPGASVKVSCKASGYAFTSYNMYWVRQAPGQSLEWMGYIDPYNGGTIYNQKLQGRVTMTVDKSTSTAYMELRSLRSDDTAVYYCARVGDAYPFAYWGQGTLVTVSS

SEQ ID NO: 248 - protein, 10 aa, synthetic construct
  source 1..10: note = D94837_1E11_1 [MICA/B]_H1L1 vhCDR1
GYAFTSYNMY

SEQ ID NO: 249 - protein, 17 aa, synthetic construct
  source 1..17: note = D94837_1E11_1 [MICA/B]_H1L1 vhCDR2
YIDPYNGGTIYNQKLQG

SEQ ID NO: 250 - protein, 9 aa, synthetic construct
  source 1..9: note = D94837_1E11_1 [MICA/B]_H1L1 vhCDR3
VGDAYPFAY

SEQ ID NO: 251 - protein, 111 aa, synthetic construct
  source 1..111: note = D94837_1E11_1 [MICA/B]_H1L1 Variable light (vl) domain
DIVLTQSPATLSLSPGERATLSCRASESVDNYGISFMNWFQQKPGQPPRLLIYAASNQGSGVPARFSGSGSGTDFTLTISSLEPEDFAVYFCQQRKEVPFTFGSGTKLEIK

SEQ ID NO: 252 - protein, 15 aa, synthetic construct
  source 1..15: note = D94837_1E11_1 [MICA/B]_H1L1 vlCDR1
RASESVDNYGISFMN

SEQ ID NO: 253 - protein, 7 aa, synthetic construct
  source 1..7: note = D94837_1E11_1 [MICA/B]_H1L1 vlCDR2
AASNQGS

SEQ ID NO: 254 - protein, 9 aa, synthetic construct
  source 1..9: note = D94837_1E11_1 [MICA/B]_H1L1 vlCDR3
QQRKEVPFT

SEQ ID NO: 255 - protein, 118 aa, synthetic construct; identical to SEQ ID NO: 247
  source 1..118: note = D94837_1E11_1 [MICA/B]_H1L2 Variable heavy (vh) domain

SEQ ID NO: 256 - protein, 10 aa, synthetic construct
  source 1..10: note = D94837_1E11_1 [MICA/B]_H1L2 vhCDR1
GYAFTSYNMY

SEQ ID NO: 257 - protein, 17 aa, synthetic construct
  source 1..17: note = D94837_1E11_1 [MICA/B]_H1L2 vhCDR2
YIDPYNGGTIYNQKLQG

SEQ ID NO: 258 - protein, 9 aa, synthetic construct
  source 1..9: note = D94837_1E11_1 [MICA/B]_H1L2 vhCDR3
VGDAYPFAY

SEQ ID NO: 259 - protein, 111 aa, synthetic construct
  source 1..111: note = D94837_1E11_1 [MICA/B]_H1L2 Variable light (vl) domain
DIVLTQSPDSLAVSLGERATINCKASESVDNYGISFMNWFQQKPGQPPKLLIYAASNQGSGVPARFSGSGSGTDFTLTISSLQAEDVAVYFCQQRKEVPFTFGSGTKLEIK

SEQ ID NO: 260 - protein, 15 aa, synthetic construct
  source 1..15: note = D94837_1E11_1 [MICA/B]_H1L2 vlCDR1
KASESVDNYGISFMN

SEQ ID NO: 261 - protein, 7 aa, synthetic construct
  source 1..7: note = D94837_1E11_1 [MICA/B]_H1L2 vlCDR2
AASNQGS

SEQ ID NO: 262 - protein, 9 aa, synthetic construct
  source 1..9: note = D94837_1E11_1 [MICA/B]_H1L2 vlCDR3
QQRKEVPFT

SEQ ID NO: 263 - protein, 118 aa, synthetic construct
  source 1..118: note = D94837_1E11_1 [MICA/B]_H2L1 Variable heavy (vh) domain
QIQLVQSGSELKKPGASVKVSCKASGYAFTSYNMYWVRQAPGQSLEWMGYIDPYNGGTIYNQGFTGRFVFSVDKSVSTAYLQISSLKAEDTAVYYCARVGDAYPFAYWGQGTLVTVSS

SEQ ID NO: 264 - protein, 10 aa, synthetic construct
  source 1..10: note = D94837_1E11_1 [MICA/B]_H2L1 vhCDR1
GYAFTSYNMY

SEQ ID NO: 265 - protein, 17 aa, synthetic construct
  source 1..17: note = D94837_1E11_1 [MICA/B]_H2L1 vhCDR2
YIDPYNGGTIYNQGFTG

SEQ ID NO: 266 - protein, 9 aa, synthetic construct
  source 1..9: note = D94837_1E11_1 [MICA/B]_H2L1 vhCDR3
VGDAYPFAY

SEQ ID NO: 267 - protein, 111 aa, synthetic construct; identical to SEQ ID NO: 251
  source 1..111: note = D94837_1E11_1 [MICA/B]_H2L1 Variable light (vl) domain

SEQ ID NO: 268 - protein, 15 aa, synthetic construct
  source 1..15: note = D94837_1E11_1 [MICA/B]_H2L1 vlCDR1
RASESVDNYGISFMN

SEQ ID NO: 269 - protein, 7 aa, synthetic construct
  source 1..7: note = D94837_1E11_1 [MICA/B]_H2L1 vlCDR2
AASNQGS

SEQ ID NO: 270 - protein, 9 aa, synthetic construct
  source 1..9: note = D94837_1E11_1 [MICA/B]_H2L1 vlCDR3
QQRKEVPFT

SEQ ID NO: 271 - protein, 118 aa, synthetic construct; identical to SEQ ID NO: 263
  source 1..118: note = D94837_1E11_1 [MICA/B]_H2L2 Variable heavy (vh) domain

SEQ ID NO: 272 - protein, 10 aa, synthetic construct
  source 1..10: note = D94837_1E11_1 [MICA/B]_H2L2 vhCDR1
GYAFTSYNMY

SEQ ID NO: 273 - protein, 17 aa, synthetic construct
  source 1..17: note = D94837_1E11_1 [MICA/B]_H2L2 vhCDR2
YIDPYNGGTIYNQGFTG

SEQ ID NO: 274 - protein, 9 aa, synthetic construct
  source 1..9: note = D94837_1E11_1 [MICA/B]_H2L2 vhCDR3
VGDAYPFAY

SEQ ID NO: 275 - protein, 111 aa, synthetic construct; identical to SEQ ID NO: 259
  source 1..111: note = D94837_1E11_1 [MICA/B]_H2L2 Variable light (vl) domain

SEQ ID NO: 276 - protein, 15 aa, synthetic construct
  source 1..15: note = D94837_1E11_1 [MICA/B]_H2L2 vlCDR1
KASESVDNYGISFMN

SEQ ID NO: 277 - protein, 7 aa, synthetic construct
  source 1..7: note = D94837_1E11_1 [MICA/B]_H2L2 vlCDR2
AASNQGS

SEQ ID NO: 278 - protein, 9 aa, synthetic construct
  source 1..9: note = D94837_1E11_1 [MICA/B]_H2L2 vlCDR3
QQRKEVPFT

SEQ ID NO: 279 - protein, 125 aa, synthetic construct
  source 1..125: note = 2E5 [MICA/B]_H0L0 Variable heavy (vh) domain
EVKLVESGGGLVQPGGSLKLSCATSGFTFSDYYMFWVRQTPEKRLEWVAYISEGGGSTYYPDTIKGRFTISRDNAKNTLYLQMSRLRSEDTAMYYCVRQRGAYNRYDDGRGFAYWGQGTLVTVSA

SEQ ID NO: 280 - protein, 10 aa, synthetic construct
  source 1..10: note = 2E5 [MICA/B]_H0L0 vhCDR1
GFTFSDYYMF

SEQ ID NO: 281 - protein, 17 aa, synthetic construct
  source 1..17: note = 2E5 [MICA/B]_H0L0 vhCDR2
YISEGGGSTYYPDTIKG

SEQ ID NO: 282 - protein, 16 aa, synthetic construct
  source 1..16: note = 2E5 [MICA/B]_H0L0 vhCDR3
QRGAYNRYDDGRGFAY

SEQ ID NO: 283 - protein, 112 aa, synthetic construct
  source 1..112: note = 2E5 [MICA/B]_H0L0 Variable light (vl) domain
DVLMTQTPLSLPVSLGDQASISCRSSQSIVHSNGNTYLEWYLQKPGQSPKLLIYKVSNRFSGVPDRLSGSGSGTDFTLKISRGEAEDLGVYYCFQGSHVPWTFGGGTKLEIK

SEQ ID NO: 284 - protein, 16 aa, synthetic construct
  source 1..16: note = 2E5 [MICA/B]_H0L0 vlCDR1
RSSQSIVHSNGNTYLE

SEQ ID NO: 285 - protein, 7 aa, synthetic construct
  source 1..7: note = 2E5 [MICA/B]_H0L0 vlCDR2
KVSNRFS

SEQ ID NO: 286 - protein, 9 aa, synthetic construct
  source 1..9: note = 2E5 [MICA/B]_H0L0 vlCDR3
FQGSHVPWT

SEQ ID NO: 287 - protein, 118 aa, synthetic construct
  source 1..118: note = 2E5 [MICA/B]_H1L1 Variable heavy (vh) domain
EVQLLESGGGLVQPGGSLRLSCATSGFTFSDYYMFWVRQAPGKRLEWVSYISEGGGSTYYPDSVKGRFTISRDNSKNTLYLQMNSLRAEDTAVYYCVRQRGAYNRYDDGRGTLVTVSS

SEQ ID NO: 288 - protein, 10 aa, synthetic construct
  source 1..10: note = 2E5 [MICA/B]_H1L1 vhCDR1
GFTFSDYYMF

SEQ ID NO: 289 - protein, 17 aa, synthetic construct
  source 1..17: note = 2E5 [MICA/B]_H1L1 vhCDR2
YISEGGGSTYYPDSVKG

SEQ ID NO: 290 - protein, 10 aa, synthetic construct
  source 1..10: note = 2E5 [MICA/B]_H1L1 vhCDR3
QRGAYNRYDD

SEQ ID NO: 291 - protein, 112 aa, synthetic construct
  source 1..112: note = 2E5 [MICA/B]_H1L1 Variable light (vl) domain
DVLMTQSPLSLPVTLGQPASISCRSSQSIVHSNGNTYLEWYQQRPGQSPRLLIYKVSNRFSGVPDRLSGSGSGTDFTLKISRVEAEDVGVYYCFQGSHVPWTFGGGTKVEIK

SEQ ID NO: 292 - protein, 16 aa, synthetic construct
  source 1..16: note = 2E5 [MICA/B]_H1L1 vlCDR1
RSSQSIVHSNGNTYLE

SEQ ID NO: 293 - protein, 7 aa, synthetic construct
  source 1..7: note = 2E5 [MICA/B]_H1L1 vlCDR2
KVSNRFS

SEQ ID NO: 294 - protein, 9 aa, synthetic construct
  source 1..9: note = 2E5 [MICA/B]_H1L1 vlCDR3
FQGSHVPWT

SEQ ID NO: 295 - protein, 118 aa, synthetic construct; identical to SEQ ID NO: 287
  source 1..118: note = 2E5 [MICA/B]_H1L2 Variable heavy (vh) domain

SEQ ID NO: 296 - protein, 10 aa, synthetic construct
  source 1..10: note = 2E5 [MICA/B]_H1L2 vhCDR1
GFTFSDYYMF

SEQ ID NO: 297 - protein, 17 aa, synthetic construct
  source 1..17: note = 2E5 [MICA/B]_H1L2 vhCDR2
YISEGGGSTYYPDSVKG

SEQ ID NO: 298 - protein, 10 aa, synthetic construct
  source 1..10: note = 2E5 [MICA/B]_H1L2 vhCDR3
QRGAYNRYDD

SEQ ID NO: 299 - protein, 112 aa, synthetic construct
  source 1..112: note = 2E5 [MICA/B]_H1L2 Variable light (vl) domain
DVLMTQSPDSLAVSLGERATINCKSSQSIVHSNGNTYLEWYQQKPGQSPKLLIYKVSNRFSGVPDRLSGSGSGTDFTLTISSLQAEDVAVYYCFQGSHVPWTFGGGTKVEIK

SEQ ID NO: 300 - protein, 16 aa, synthetic construct
  source 1..16: note = 2E5 [MICA/B]_H1L2 vlCDR1
KSSQSIVHSNGNTYLE

SEQ ID NO: 301 - protein, 7 aa, synthetic construct
  source 1..7: note = 2E5 [MICA/B]_H1L2 vlCDR2
KVSNRFS

SEQ ID NO: 302 - protein, 9 aa, synthetic construct
  source 1..9: note = 2E5 [MICA/B]_H1L2 vlCDR3
FQGSHVPWT

SEQ ID NO: 303 - protein, 118 aa, synthetic construct
  source 1..118: note = 2E5 [MICA/B]_H2L1 Variable heavy (vh) domain
QVQLVQSGAEVKKPGASVKVSCKTSGFTFSDYYMFWVRQAPGQRLEWMGYISEGGGSTYYPDKFQGRVTMTRDNSKNTLYMELSSLRSEDTAVYYCVRQRGAYNRYDDGRGTLVTVSS

SEQ ID NO: 304 - protein, 10 aa, synthetic construct
  source 1..10: note = 2E5 [MICA/B]_H2L1 vhCDR1
GFTFSDYYMF

SEQ ID NO: 305 - protein, 17 aa, synthetic construct
  source 1..17: note = 2E5 [MICA/B]_H2L1 vhCDR2
YISEGGGSTYYPDKFQG

SEQ ID NO: 306 - protein, 10 aa, synthetic construct
  source 1..10: note = 2E5 [MICA/B]_H2L1 vhCDR3
QRGAYNRYDD

SEQ ID NO: 307 - protein, 112 aa, synthetic construct; identical to SEQ ID NO: 291
  source 1..112: note = 2E5 [MICA/B]_H2L1 Variable light (vl) domain

SEQ ID NO: 308 - protein, 16 aa, synthetic construct
  source 1..16: note = 2E5 [MICA/B]_H2L1 vlCDR1
RSSQSIVHSNGNTYLE

SEQ ID NO: 309 - protein, 7 aa, synthetic construct
  source 1..7: note = 2E5 [MICA/B]_H2L1 vlCDR2
KVSNRFS

SEQ ID NO: 310 - protein, 9 aa, synthetic construct
  source 1..9: note = 2E5 [MICA/B]_H2L1 vlCDR3
FQGSHVPWT

SEQ ID NO: 311 - protein, 118 aa, synthetic construct; identical to SEQ ID NO: 303
  source 1..118: note = 2E5 [MICA/B]_H2L2 Variable heavy (vh) domain

SEQ ID NO: 312 - protein, 10 aa, synthetic construct
  source 1..10: note = 2E5 [MICA/B]_H2L2 vhCDR1
GFTFSDYYMF

SEQ ID NO: 313 - protein, 17 aa, synthetic construct
  source 1..17: note = 2E5 [MICA/B]_H2L2 vhCDR2
YISEGGGSTYYPDKFQG

SEQ ID NO: 314 - protein, 10 aa, synthetic construct
  source 1..10: note = 2E5 [MICA/B]_H2L2 vhCDR3
QRGAYNRYDD

SEQ ID NO: 315 - protein, 112 aa, synthetic construct; identical to SEQ ID NO: 299
  source 1..112: note = 2E5 [MICA/B]_H2L2 Variable light (vl) domain

SEQ ID NO: 316 - protein, 16 aa, synthetic construct
  source 1..16: note = 2E5 [MICA/B]_H2L2 vlCDR1
KSSQSIVHSNGNTYLE

SEQ ID NO: 317 - protein, 7 aa, synthetic construct
  source 1..7: note = 2E5 [MICA/B]_H2L2 vlCDR2
KVSNRFS

SEQ ID NO: 318 - protein, 9 aa, synthetic construct
  source 1..9: note = 2E5 [MICA/B]_H2L2 vlCDR3
FQGSHVPWT

SEQ ID NO: 319 - protein, 117 aa, synthetic construct
  source 1..117: note = D94852_2E12 [MICA/B]_H0L0 Variable heavy (vh) domain
QVQLQQSGAELVRPGTSVKVSCKASGYAFTNYLIEWVKQRPGQGLEWIGGINPGSGSTNYNEKFKGKATLTADKSSSTAYMQLSTLTFDDSAVYFCARLGGNYVDYWGQGTTLTVSS

SEQ ID NO: 320 - protein, 10 aa, synthetic construct
  source 1..10: note = D94852_2E12 [MICA/B]_H0L0 vhCDR1
GYAFTNYLIE

SEQ ID NO: 321 - protein, 17 aa, synthetic construct
  source 1..17: note = D94852_2E12 [MICA/B]_H0L0 vhCDR2
GINPGSGSTNYNEKFKG

SEQ ID NO: 322 - protein, 8 aa, synthetic construct
  source 1..8: note = D94852_2E12 [MICA/B]_H0L0 vhCDR3
LGGNYVDY

SEQ ID NO: 323 - protein, 107 aa, synthetic construct
  source 1..107: note = D94852_2E12 [MICA/B]_H0L0 Variable light (vl) domain
DIQMTQSPASLSASVGETVTITCRASGNIHNYLAWYQQKQGKSPQLLVYNAKTLADGVPSRFSGSGSGTQYSLKINSLQPEDFGSYYCQHFWSTPRTFGGGTKLEIK

SEQ ID NO: 324 - protein, 11 aa, synthetic construct
  source 1..11: note = D94852_2E12 [MICA/B]_H0L0 vlCDR1
RASGNIHNYLA

SEQ ID NO: 325 - protein, 7 aa, synthetic construct
  source 1..7: note = D94852_2E12 [MICA/B]_H0L0 vlCDR2
NAKTLAD

SEQ ID NO: 326 - protein, 9 aa, synthetic construct
  source 1..9: note = D94852_2E12 [MICA/B]_H0L0 vlCDR3
QHFWSTPRT

SEQ ID NO: 327 - protein, 117 aa, synthetic construct
  source 1..117: note = D94852_2E12 [MICA/B]_H1L1 Variable heavy (vh) domain
QVQLVQSGAEVKKPGASVKVSCKASGYAFTNYLIEWVRQAPGQGLEWMGGINPGSGSTNYNEKFQGRVTMTADKSISTAYMELSRLRSDDTAVYFCARLGGNYVDYWGQGTLVTVSS

SEQ ID NO: 328 - protein, 10 aa, synthetic construct
  source 1..10: note = D94852_2E12 [MICA/B]_H1L1 vhCDR1
GYAFTNYLIE

SEQ ID NO: 329 - protein, 17 aa, synthetic construct
  source 1..17: note = D94852_2E12 [MICA/B]_H1L1 vhCDR2
GINPGSGSTNYNEKFQG

SEQ ID NO: 330 - protein, 8 aa, synthetic construct
  source 1..8: note = D94852_2E12 [MICA/B]_H1L1 vhCDR3
LGGNYVDY

SEQ ID NO: 331 - protein, 107 aa, synthetic construct
  source 1..107: note = D94852_2E12 [MICA/B]_H1L1 Variable light (vl) domain
DIQMTQSPSSLSASVGDRVTITCRASGNIHNYLAWYQQKPGKSPKLLVYNAKTLADGVPSRFSGSGSGTDYTLTISSLQPEDVATYYCQHFWSTPRTFGGGTKVEIK

SEQ ID NO: 332 - protein, 11 aa, synthetic construct
  source 1..11: note = D94852_2E12 [MICA/B]_H1L1 vlCDR1
RASGNIHNYLA

SEQ ID NO: 333 - protein, 7 aa, synthetic construct
  source 1..7: note = D94852_2E12 [MICA/B]_H1L1 vlCDR2
NAKTLAD

SEQ ID NO: 334 - protein, 9 aa, synthetic construct
  source 1..9: note = D94852_2E12 [MICA/B]_H1L1 vlCDR3
QHFWSTPRT

SEQ ID NO: 335 - protein, 117 aa, synthetic construct; identical to SEQ ID NO: 327
  source 1..117: note = D94852_2E12 [MICA/B]_H1L2 Variable heavy (vh) domain

SEQ ID NO: 336 - protein, 10 aa, synthetic construct
  source 1..10: note = D94852_2E12 [MICA/B]_H1L2 vhCDR1
GYAFTNYLIE

SEQ ID NO: 337 - protein, 17 aa, synthetic construct
  source 1..17: note = D94852_2E12 [MICA/B]_H1L2 vhCDR2
GINPGSGSTNYNEKFQG

SEQ ID NO: 338 - protein, 8 aa, synthetic construct
  source 1..8: note = D94852_2E12 [MICA/B]_H1L2 vhCDR3
LGGNYVDY

SEQ ID NO: 339 - protein, 107 aa, synthetic construct
  source 1..107: note = D94852_2E12 [MICA/B]_H1L2 Variable light (vl) domain
DIQMTQSPDSLAVSLGERATINCKASGNIHNYLAWYQQKPGQSPKLLVYNAKTLADGVPSRFSGSGSGTDYTLTISSLQAEDVAVYYCQHFWSTPRTFGGGTKVEIK

SEQ ID NO: 340 - protein, 11 aa, synthetic construct
  source 1..11: note = D94852_2E12 [MICA/B]_H1L2 vlCDR1
KASGNIHNYLA

SEQ ID NO: 341 - protein, 7 aa, synthetic construct
  source 1..7: note = D94852_2E12 [MICA/B]_H1L2 vlCDR2
NAKTLAD

SEQ ID NO: 342 - protein, 9 aa, synthetic construct
  source 1..9: note = D94852_2E12 [MICA/B]_H1L2 vlCDR3
QHFWSTPRT

SEQ ID NO: 343 - protein, 117 aa, synthetic construct
  source 1..117: note = D94852_2E12 [MICA/B]_H2L1 Variable heavy (vh) domain
EVQLVQSGAEVKKPGESLRISCKASGYAFTNYLIEWVRQMPGKGLEWMGGINPGSGSTNYAESFQGHVTISADKSISTAYLQWSSLKASDTAMYFCARLGGNYVDYWGQGTLVTVSS

SEQ ID NO: 344 - protein, 10 aa, synthetic construct
  source 1..10: note = D94852_2E12 [MICA/B]_H2L1 vhCDR1
GYAFTNYLIE

SEQ ID NO: 345 - protein, 17 aa, synthetic construct
  source 1..17: note = D94852_2E12 [MICA/B]_H2L1 vhCDR2
GINPGSGSTNYAESFQG

SEQ ID NO: 346 - protein, 8 aa, synthetic construct
  source 1..8: note = D94852_2E12 [MICA/B]_H2L1 vhCDR3
LGGNYVDY

SEQ ID NO: 347 - protein, 107 aa, synthetic construct; identical to SEQ ID NO: 331
  source 1..107: note = D94852_2E12 [MICA/B]_H2L1 Variable light (vl) domain

SEQ ID NO: 348 - protein, 11 aa, synthetic construct
  source 1..11: note = D94852_2E12 [MICA/B]_H2L1 vlCDR1
RASGNIHNYLA

SEQ ID NO: 349 - protein, 7 aa, synthetic construct
  source 1..7: note = D94852_2E12 [MICA/B]_H2L1 vlCDR2
NAKTLAD

SEQ ID NO: 350 - protein, 9 aa, synthetic construct
  source 1..9: note = D94852_2E12 [MICA/B]_H2L1 vlCDR3
QHFWSTPRT

SEQ ID NO: 351 - protein, 117 aa, synthetic construct; identical to SEQ ID NO: 343
  source 1..117: note = D94852_2E12 [MICA/B]_H2L2 Variable heavy (vh) domain

SEQ ID NO: 352 - protein, 10 aa, synthetic construct
  source 1..10: note = D94852_2E12 [MICA/B]_H2L2 vhCDR1
GYAFTNYLIE

SEQ ID NO: 353 - protein, 17 aa, synthetic construct
  source 1..17: note = D94852_2E12 [MICA/B]_H2L2 vhCDR2
GINPGSGSTNYAESFQG

SEQ ID NO: 354 - protein, 8 aa, synthetic construct
  source 1..8: note = D94852_2E12 [MICA/B]_H2L2 vhCDR3
LGGNYVDY

SEQ ID NO: 355 - protein, 107 aa, synthetic construct; identical to SEQ ID NO: 339
  source 1..107: note = D94852_2E12 [MICA/B]_H2L2 Variable light (vl) domain

SEQ ID NO: 356 - protein, 11 aa, synthetic construct
  source 1..11: note = D94852_2E12 [MICA/B]_H2L2 vlCDR1
KASGNIHNYLA

SEQ ID NO: 357 - protein, 7 aa, synthetic construct
  source 1..7: note = D94852_2E12 [MICA/B]_H2L2 vlCDR2
NAKTLAD

SEQ ID NO: 358 - protein, 9 aa, synthetic construct
  source 1..9: note = D94852_2E12 [MICA/B]_H2L2 vlCDR3
QHFWSTPRT

SEQ ID NO: 359 - protein, 123 aa, synthetic construct
  source 1..123: note = D99136_2F7 [MICA/B]_H0L0 Variable heavy (vh) domain
QVHLKQSGAELVRPGASVKLSCKASGYTFTDYYIDWMNQRPGQGLEWIARIYPGSGNSYYNEKFKGKATLTAEKSASTAHMQLSSLTSEDSAVYFCAREGPTGVAPHWYFDVWGTGTTVTVSS

SEQ ID NO: 360 - protein, 10 aa, synthetic construct
  source 1..10: note = D99136_2F7 [MICA/B]_H0L0 vhCDR1
GYTFTDYYID

SEQ ID NO: 361 - protein, 17 aa, synthetic construct
  source 1..17: note = D99136_2F7 [MICA/B]_H0L0 vhCDR2
RIYPGSGNSYYNEKFKG

SEQ ID NO: 362 - protein, 14 aa, synthetic construct
  source 1..14: note = D99136_2F7 [MICA/B]_H0L0 vhCDR3
EGPTGVAPHWYFDV

SEQ ID NO: 363 - protein, 107 aa, synthetic construct
  source 1..107: note = D99136_2F7 [MICA/B]_H0L0 Variable light (vl) domain
DIVMSQSPSSLSSYLGDRVTISCRASQDIGNYLNWYQQKPDGTVKLLIYSTSRLHSGVPSRFSGSGSGTDYSLTISNLEQEDVATYFCQQGNTLPWTFGGGTKLEIK

SEQ ID NO: 364 - protein, 11 aa, synthetic construct
  source 1..11: note = D99136_2F7 [MICA/B]_H0L0 vlCDR1
RASQDIGNYLN

SEQ ID NO: 365 - protein, 7 aa, synthetic construct
  source 1..7: note = D99136_2F7 [MICA/B]_H0L0 vlCDR2
STSRLHS

SEQ ID NO: 366 - protein, 9 aa, synthetic construct
  source 1..9: note = D99136_2F7 [MICA/B]_H0L0 vlCDR3
QQGNTLPWT

SEQ ID NO: 367 - protein, 123 aa, synthetic construct
  source 1..123: note = D99136_2F7 [MICA/B]_H1L1 Variable heavy (vh) domain
QVQLVQSGAEVKKPGASVKVSCKASGYTFTDYYIDWMRQAPGQGLEWMGRIYPGSGNSYYNEKFQGRVTMTADKSISTAYMELSRLRSDDTAVYFCAREGPTGVAPHWYFDVWGTGTLVTVSS

SEQ ID NO: 368 - protein, 10 aa, synthetic construct
  source 1..10: note = D99136_2F7 [MICA/B]_H1L1 vhCDR1
GYTFTDYYID

SEQ ID NO: 369 - protein, 17 aa, synthetic construct
  source 1..17: note = D99136_2F7 [MICA/B]_H1L1 vhCDR2
RIYPGSGNSYYNEKFQG

SEQ ID NO: 370 - protein, 14 aa, synthetic construct
  source 1..14: note = D99136_2F7 [MICA/B]_H1L1 vhCDR3
EGPTGVAPHWYFDV

SEQ ID NO: 371 - protein, 107 aa, synthetic construct
  source 1..107: note = D99136_2F7 [MICA/B]_H1L1 Variable light (vl) domain
DIVMTQSPSSLSASVGDRVTITCQASQDIGNYLNWYQQKPGKTVKLLIYSTSRLHSGVPSRFSGSGSGTDYTFTISSLQPEDIATYFCQQGNTLPWTFGGGTKVEIK

SEQ ID NO: 372 - protein, 11 aa, synthetic construct
  source 1..11: note = D99136_2F7 [MICA/B]_H1L1 vlCDR1
QASQDIGNYLN

SEQ ID NO: 373 - protein, 7 aa, synthetic construct
  source 1..7: note = D99136_2F7 [MICA/B]_H1L1 vlCDR2
STSRLHS

SEQ ID NO: 374 - protein, 9 aa, synthetic construct
  source 1..9: note = D99136_2F7 [MICA/B]_H1L1 vlCDR3
QQGNTLPWT

SEQ ID NO: 375 - protein, 123 aa, synthetic construct; identical to SEQ ID NO: 367
  source 1..123: note = D99136_2F7 [MICA/B]_H1L2 Variable heavy (vh) domain

SEQ ID NO: 376 - protein, 10 aa, synthetic construct
  source 1..10: note = D99136_2F7 [MICA/B]_H1L2 vhCDR1
GYTFTDYYID

SEQ ID NO: 377 - protein, 17 aa, synthetic construct
  source 1..17: note = D99136_2F7 [MICA/B]_H1L2 vhCDR2
RIYPGSGNSYYNEKFQG

SEQ ID NO: 378 - protein, 14 aa, synthetic construct
  source 1..14: note = D99136_2F7 [MICA/B]_H1L2 vhCDR3
EGPTGVAPHWYFDV

SEQ ID NO: 379 - protein, 107 aa, synthetic construct
  source 1..107: note = D99136_2F7 [MICA/B]_H1L2 Variable light (vl) domain
DIVMTQSPDSLAVSLGERATINCKASQDIGNYLNWYQQKPGQTVKLLIYSTSRLHSGVPSRFSGSGSGTDYTLTISSLQAEDVAVYFCQQGNTLPWTFGGGTKVEIK

SEQ ID NO: 380 - protein, 11 aa, synthetic construct
  source 1..11: note = D99136_2F7 [MICA/B]_H1L2 vlCDR1
KASQDIGNYLN

SEQ ID NO: 381 - protein, 7 aa, synthetic construct
  source 1..7: note = D99136_2F7 [MICA/B]_H1L2 vlCDR2
STSRLHS

SEQ ID NO: 382 - protein, 9 aa, synthetic construct
  source 1..9: note = D99136_2F7 [MICA/B]_H1L2 vlCDR3
QQGNTLPWT

SEQ ID NO: 383 - protein, 123 aa, synthetic construct
  source 1..123: note = D99136_2F7 [MICA/B]_H2L1 Variable heavy (vh) domain
QVQLVQSGSELKKPGASVKVSCKASGYTFTDYYIDWMRQAPGQGLEWMGRIYPGSGNSYYNEGFTGRFVFSADKSVSTAYLQISSLKAEDTAVYFCAREGPTGVAPHWYFDVWGTGTLVTVSS

SEQ ID NO: 384 - protein, 10 aa, synthetic construct
  source 1..10: note = D99136_2F7 [MICA/B]_H2L1 vhCDR1
GYTFTDYYID

SEQ ID NO: 385 - protein, 17 aa, synthetic construct
  source 1..17: note = D99136_2F7 [MICA/B]_H2L1 vhCDR2
RIYPGSGNSYYNEGFTG

SEQ ID NO: 386 - protein, 14 aa, synthetic construct
  source 1..14: note = D99136_2F7 [MICA/B]_H2L1 vhCDR3
EGPTGVAPHWYFDV

SEQ ID NO: 387 - protein, 107 aa, synthetic construct; identical to SEQ ID NO: 371
  source 1..107: note = D99136_2F7 [MICA/B]_H2L1 Variable light (vl) domain

SEQ ID NO: 388 - protein, 11 aa, synthetic construct
  source 1..11: note = D99136_2F7 [MICA/B]_H2L1 vlCDR1
QASQDIGNYLN

SEQ ID NO: 389 - protein, 7 aa, synthetic construct
  source 1..7: note = D99136_2F7 [MICA/B]_H2L1 vlCDR2
STSRLHS

SEQ ID NO: 390 - protein, 9 aa, synthetic construct
  source 1..9: note = D99136_2F7 [MICA/B]_H2L1 vlCDR3
QQGNTLPWT

SEQ ID NO: 391 - protein, 123 aa, synthetic construct; identical to SEQ ID NO: 383
  source 1..123: note = D99136_2F7 [MICA/B]_H2L2 Variable heavy (vh) domain

SEQ ID NO: 392 - protein, 10 aa, synthetic construct
  source 1..10: note = D99136_2F7 [MICA/B]_H2L2 vhCDR1
GYTFTDYYID

SEQ ID NO: 393 - protein, 17 aa, synthetic construct
  source 1..17: note = D99136_2F7 [MICA/B]_H2L2 vhCDR2
RIYPGSGNSYYNEGFTG

SEQ ID NO: 394 - protein, 14 aa, synthetic construct
  source 1..14: note = D99136_2F7 [MICA/B]_H2L2 vhCDR3
EGPTGVAPHWYFDV

SEQ ID NO: 395 - protein, 107 aa, synthetic construct; identical to SEQ ID NO: 379
  source 1..107: note = D99136_2F7 [MICA/B]_H2L2 Variable light (vl) domain

SEQ ID NO: 396 - protein, 11 aa, synthetic construct
  source 1..11: note = D99136_2F7 [MICA/B]_H2L2 vlCDR1
KASQDIGNYLN

SEQ ID NO: 397 - protein, 7 aa, synthetic construct
  source 1..7: note = D99136_2F7 [MICA/B]_H2L2 vlCDR2
STSRLHS

SEQ ID NO: 398 - protein, 9 aa, synthetic construct
  source 1..9: note = D99136_2F7 [MICA/B]_H2L2 vlCDR3
QQGNTLPWT

SEQ ID NO: 399 - protein, 117 aa, synthetic construct
  source 1..117: note = D103388_1C7 [MICA/B]_H0L0 Variable heavy (vh) domain
QVQLRQSGPGLGQPSQSLSITCTVFGFSLTNYGVHWVRQSPGKGLEWLGMIWNDGFTDFNAAFISRLSISKDNSKSQVFFKMNSLQPSDTAIYYCASPYFHYAMDYWGQGTSVTVSS

SEQ ID NO: 400 - protein, 10 aa, synthetic construct
  source 1..10: note = D103388_1C7 [MICA/B]_H0L0 vhCDR1
GFSLTNYGVH

SEQ ID NO: 401 - protein, 16 aa, synthetic construct
  source 1..16: note = D103388_1C7 [MICA/B]_H0L0 vhCDR2
MIWNDGFTDFNAAFIS

SEQ ID NO: 402 - protein, 9 aa, synthetic construct
  source 1..9: note = D103388_1C7 [MICA/B]_H0L0 vhCDR3
PYFHYAMDY

SEQ ID NO: 403 - protein, 108 aa, synthetic construct
  source 1..108: note = D103388_1C7 [MICA/B]_H0L0 Variable light (vl) domain
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPFTFGSGTKLEIK

SEQ ID NO: 404 - protein, 12 aa, synthetic construct
  source 1..12: note = D103388_1C7 [MICA/B]_H0L0 vlCDR1
TASSSVSSSYLH

SEQ ID NO: 405 - protein, 7 aa, synthetic construct
  source 1..7: note = D103388_1C7 [MICA/B]_H0L0 vlCDR2
STSNLAS

SEQ ID NO: 406 - protein, 9 aa, synthetic construct
  source 1..9: note = D103388_1C7 [MICA/B]_H0L0 vlCDR3
HQYHRSPFT

SEQ ID NO: 407 - protein, 117 aa, synthetic construct
  source 1..117: note = D103388_1C7 [MICA/B]_H1L1 Variable heavy (vh) domain
QVQLVQSGAEVKKPGASVKVSCKVSGFSLTNYGVHWVRQAPGQGLEWMGMIWNDGFTDYNAKFQGRVTMTKDNSISTVYMELSRLRSDDTAVYYCASPYFHYAMDYWGQGTTVTVSS

SEQ ID NO: 408 - protein, 10 aa, synthetic construct
  source 1..10: note = D103388_1C7 [MICA/B]_H1L1 vhCDR1
GFSLTNYGVH

SEQ ID NO: 409 - protein, 16 aa, synthetic construct
  source 1..16: note = D103388_1C7 [MICA/B]_H1L1 vhCDR2
MIWNDGFTDYNAKFQG

SEQ ID NO: 410 - protein, 9 aa, synthetic construct
  source 1..9: note = D103388_1C7 [MICA/B]_H1L1 vhCDR3
PYFHYAMDY

SEQ ID NO: 411 - protein, 108 aa, synthetic construct
  source 1..108: note = D103388_1C7 [MICA/B]_H1L1 Variable light (vl) domain
QIVLTQSPATLSLSPGERATLSCRASSSVSSSYLHWYQQKPGQSPRLLIYSTSNLASGVPARFSGSGSGTDYTLTISSLEPEDFAVYYCHQYHRSPFTFGSGTKLEIK

SEQ ID NO: 412 - protein, 12 aa, synthetic construct
  source 1..12: note = D103388_1C7 [MICA/B]_H1L1 vlCDR1
RASSSVSSSYLH

SEQ ID NO: 413 - protein, 7 aa, synthetic construct
  source 1..7: note = D103388_1C7 [MICA/B]_H1L1 vlCDR2
STSNLAS

SEQ ID NO: 414 - protein, 9 aa, synthetic construct
  source 1..9: note = D103388_1C7 [MICA/B]_H1L1 vlCDR3
HQYHRSPFT

SEQ ID NO: 415 - protein, 117 aa, synthetic construct; identical to SEQ ID NO: 407
  source 1..117: note = D103388_1C7 [MICA/B]_H1L2 Variable heavy (vh) domain

SEQ ID NO: 416 - protein, 10 aa, synthetic construct
  source 1..10: note = D103388_1C7 [MICA/B]_H1L2 vhCDR1
GFSLTNYGVH

SEQ ID NO: 417 - protein, 16 aa, synthetic construct
  source 1..16: note = D103388_1C7 [MICA/B]_H1L2 vhCDR2
MIWNDGFTDYNAKFQG

SEQ ID NO: 418 - protein, 9 aa, synthetic construct
  source 1..9: note = D103388_1C7 [MICA/B]_H1L2 vhCDR3
PYFHYAMDY

SEQ ID NO: 419 - protein, 108 aa, synthetic construct
  source 1..108: note = D103388_1C7 [MICA/B]_H1L2 Variable light (vl) domain
QIVLTQSPFSLSASVGDRVTITCWASSSVSSSYLHWYQQKPAKSPKLFIYSTSNLASGVPARFSGSGSGTDYTLTISSLQPEDFATYYCHQYHRSPFTFGSGTKLEIK

SEQ ID NO: 420 - protein, 12 aa, synthetic construct
  source 1..12: note = D103388_1C7 [MICA/B]_H1L2 vlCDR1
WASSSVSSSYLH

SEQ ID NO: 421 - protein, 7 aa, synthetic construct
  source 1..7: note = D103388_1C7 [MICA/B]_H1L2 vlCDR2
STSNLAS

SEQ ID NO: 422 - protein, 9 aa, synthetic construct
  source 1..9: note = D103388_1C7 [MICA/B]_H1L2 vlCDR3
HQYHRSPFT

SEQ ID NO: 423 - protein, 117 aa, synthetic construct
  source 1..117: note = D103388_1C7 [MICA/B]_H2L1 Variable heavy (vh) domain
EVQLVESGGGLVKPGGSLRLSCAVSGFSLTNYGVHWVRQAPGKGLEWVGMIWNDGFTDYNAPVKGRFTISKDNSKSTVYLQMNSLKTEDTAVYYCASPYFHYAMDYWGQGTTVTVSS

SEQ ID NO: 424 - protein, 10 aa, synthetic construct
  source 1..10: note = D103388_1C7 [MICA/B]_H2L1 vhCDR1
GFSLTNYGVH

SEQ ID NO: 425 - protein, 16 aa, synthetic construct
  source 1..16: note = D103388_1C7 [MICA/B]_H2L1 vhCDR2
MIWNDGFTDYNAPVKG

SEQ ID NO: 426 - protein, 9 aa, synthetic construct
  source 1..9: note = D103388_1C7 [MICA/B]_H2L1 vhCDR3
PYFHYAMDY

SEQ ID NO: 427 - protein, 108 aa, synthetic construct; identical to SEQ ID NO: 411
  source 1..108: note = D103388_1C7 [MICA/B]_H2L1 Variable light (vl) domain

SEQ ID NO: 428 - protein, 12 aa, synthetic construct
  source 1..12: note = D103388_1C7 [MICA/B]_H2L1 vlCDR1
RASSSVSSSYLH

SEQ ID NO: 429 - protein, 7 aa, synthetic construct
  source 1..7: note = D103388_1C7 [MICA/B]_H2L1 vlCDR2
STSNLAS

SEQ ID NO: 430 - protein, 9 aa, synthetic construct
  source 1..9: note = D103388_1C7 [MICA/B]_H2L1 vlCDR3
HQYHRSPFT

SEQ ID NO: 431 - protein, 117 aa, synthetic construct; identical to SEQ ID NO: 423
  source 1..117: note = D103388_1C7 [MICA/B]_H2L2 Variable heavy (vh) domain

SEQ ID NO: 432 - protein, 10 aa, synthetic construct
  source 1..10: note = D103388_1C7 [MICA/B]_H2L2 vhCDR1
GFSLTNYGVH

SEQ ID NO: 433 - protein, 16 aa, synthetic construct
  source 1..16: note = D103388_1C7 [MICA/B]_H2L2 vhCDR2
MIWNDGFTDYNAPVKG

SEQ ID NO: 434 - protein, 9 aa, synthetic construct
  source 1..9: note = D103388_1C7 [MICA/B]_H2L2 vhCDR3
PYFHYAMDY

SEQ ID NO: 435 - protein, 108 aa, synthetic construct; identical to SEQ ID NO: 419
  source 1..108: note = D103388_1C7 [MICA/B]_H2L2 Variable light (vl) domain

SEQ ID NO: 436 - protein, 12 aa, synthetic construct
  source 1..12: note = D103388_1C7 [MICA/B]_H2L2 vlCDR1
WASSSVSSSYLH

SEQ ID NO: 437 - protein, 7 aa, synthetic construct
  source 1..7: note = D103388_1C7 [MICA/B]_H2L2 vlCDR2
STSNLAS

SEQ ID NO: 438 - protein, 9 aa, synthetic construct
  source 1..9: note = D103388_1C7 [MICA/B]_H2L2 vlCDR3
HQYHRSPFT

SEQ ID NO: 439 - protein, 116 aa, synthetic construct
  source 1..116: note = D103388_1D7 [MICA/B]_H0L0 Variable heavy (vh) domain
QVQLQQSGAELMKPGASVKISCKATGYSFTSYYIEWVRQRPGHGLEWIGEIFPGNGDTNYNVNFKGKASLTAYTSSNTVYLQLSSLTSEDSAVYYCTRGHYNMDYWGQGTSVTVSS

SEQ ID NO: 440 - protein, 10 aa, synthetic construct
  source 1..10: note = D103388_1D7 [MICA/B]_H0L0 vhCDR1
GYSFTSYYIE

SEQ ID NO: 441 - protein, 17 aa, synthetic construct
  source 1..17: note = D103388_1D7 [MICA/B]_H0L0 vhCDR2
EIFPGNGDTNYNVNFKG

SEQ ID NO: 442 - protein, 7 aa, synthetic construct
  source 1..7: note = D103388_1D7 [MICA/B]_H0L0 vhCDR3
GHYNMDY

SEQ ID NO: 443 - protein, 107 aa, synthetic construct
  source 1..107: note = D103388_1D7 [MICA/B]_H0L0 Variable light (vl) domain
DIQMTQTTSSLSASLGDRVTISCRASQDISNYSNWYQQKPDGSVKLLIYYTSRLHSGVPSRFSGSGSGTDYSLTISNLDQEDIATYFCQQGNTLPRTFGGGTKLEIK

SEQ ID NO: 444 - protein, 11 aa, synthetic construct
  source 1..11: note = D103388_1D7 [MICA/B]_H0L0 vlCDR1
RASQDISNYSN

SEQ ID NO: 445 - protein, 7 aa, synthetic construct
  source 1..7: note = D103388_1D7 [MICA/B]_H0L0 vlCDR2
YTSRLHS

SEQ ID NO: 446 - protein, 9 aa, synthetic construct
  source 1..9: note = D103388_1D7 [MICA/B]_H0L0 vlCDR3
QQGNTLPRT

SEQ ID NO: 447 - protein, 116 aa, synthetic construct
  source 1..116: note = D103388_1D7 [MICA/B]_H1L1 Variable heavy (vh) domain
QVQLVQSGAEVKKPGASVKVSCKASGYSFTSYYIEWVRQAPGQGLEWMGEIFPGNGDTNYNVKFQGRVTMTADTSTNTVYMELSSLRSEDTAVYYCTRGHYNMDYWGQGTTVTVSS

SEQ ID NO: 448 - protein, 10 aa, synthetic construct
  source 1..10: note = D103388_1D7 [MICA/B]_H1L1 vhCDR1
GYSFTSYYIE

SEQ ID NO: 449 - protein, 17 aa, synthetic construct
  source 1..17: note = D103388_1D7 [MICA/B]_H1L1 vhCDR2
EIFPGNGDTNYNVKFQG

SEQ ID NO: 450 - protein, 7 aa, synthetic construct
  source 1..7: note = D103388_1D7 [MICA/B]_H1L1 vhCDR3
GHYNMDY

SEQ ID NO: 451 - protein, 107 aa, synthetic construct
  source 1..107: note = D103388_1D7 [MICA/B]_H1L1 Variable light (vl) domain
DIQMTQSPSSLSASVGDRVTITCQASQDISNYLNWYQQKPGKSVKLLIYYTSRLHSGVPSRFSGSGSGTDYTFTISSLQPEDIATYFCQQGNTLPRTFGGGTKVEIK

SEQ ID NO: 452 - protein, 11 aa, synthetic construct
  source 1..11: note = D103388_1D7 [MICA/B]_H1L1 vlCDR1
QASQDISNYLN

SEQ ID NO: 453 - protein, 7 aa, synthetic construct
  source 1..7: note = D103388_1D7 [MICA/B]_H1L1 vlCDR2
YTSRLHS

SEQ ID NO: 454 - protein, 9 aa, synthetic construct
  source 1..9: note = D103388_1D7 [MICA/B]_H1L1 vlCDR3
QQGNTLPRT

SEQ ID NO: 455 - protein, 116 aa, synthetic construct; identical to SEQ ID NO: 447
  source 1..116: note = D103388_1D7 [MICA/B]_H1L2 Variable heavy (vh) domain

SEQ ID NO: 456 - protein, 10 aa, synthetic construct
  source 1..10: note = D103388_1D7 [MICA/B]_H1L2 vhCDR1
GYSFTSYYIE

SEQ ID NO: 457 - protein, 17 aa, synthetic construct
  source 1..17: note = D103388_1D7 [MICA/B]_H1L2 vhCDR2
EIFPGNGDTNYNVKFQG

SEQ ID NO: 458 - protein, 7 aa, synthetic construct
  source 1..7: note = D103388_1D7 [MICA/B]_H1L2 vhCDR3
GHYNMDY

SEQ ID NO: 459 - protein, 107 aa, synthetic construct
  source 1..107: note = D103388_1D7 [MICA/B]_H1L2 Variable light (vl) domain
DIQMTQSPDSLAVSLGERATINCKASQDISNYLNWYQQKPGQSVKLLIYYTSRLHSGVPSRFSGSGSGTDYTLTISSLQAEDVAVYFCQQGNTLPRTFGGGTKVEIK

SEQ ID NO: 460 - protein, 11 aa, synthetic construct
  source 1..11: note = D103388_1D7 [MICA/B]_H1L2 vlCDR1
KASQDISNYLN

SEQ ID NO: 461 - protein, 7 aa, synthetic construct
  source 1..7: note = D103388_1D7 [MICA/B]_H1L2 vlCDR2
YTSRLHS

SEQ ID NO: 462 - protein, 9 aa, synthetic construct
  source 1..9: note = D103388_1D7 [MICA/B]_H1L2 vlCDR3
QQGNTLPRT

SEQ ID NO: 463 - protein, 116 aa, synthetic construct
  source 1..116: note = D103388_1D7 [MICA/B]_H2L1 Variable heavy (vh) domain
QVQLVQSGSELKKPGASVKVSCKASGYSFTSYYIEWVRQAPGQGLEWMGEIFPGNGDTNYNVGFTGRFVFSADTSVNTVYLQISSLKAEDTAVYYCTRGHYNMDYWGQGTTVTVSS

SEQ ID NO: 464 - protein, 10 aa, synthetic construct
  source 1..10: note = D103388_1D7 [MICA/B]_H2L1 vhCDR1
GYSFTSYYIE

SEQ ID NO: 465 - protein, 17 aa, synthetic construct
  source 1..17: note = D103388_1D7 [MICA/B]_H2L1 vhCDR2
EIFPGNGDTNYNVGFTG

SEQ ID NO: 466 - protein, 7 aa, synthetic construct
  source 1..7: note = D103388_1D7 [MICA/B]_H2L1 vhCDR3
GHYNMDY

SEQ ID NO: 467 - protein, 107 aa, synthetic construct; identical to SEQ ID NO: 451
  source 1..107: note = D103388_1D7 [MICA/B]_H2L1 Variable light (vl) domain

SEQ ID NO: 468 - protein, 11 aa, synthetic construct
  source 1..11: note = D103388_1D7 [MICA/B]_H2L1 vlCDR1
QASQDISNYLN

SEQ ID NO: 469 - protein, 7 aa, synthetic construct
  source 1..7: note = D103388_1D7 [MICA/B]_H2L1 vlCDR2
YTSRLHS

SEQ ID NO: 470 - protein, 9 aa, synthetic construct
  source 1..9: note = D103388_1D7 [MICA/B]_H2L1 vlCDR3
QQGNTLPRT

SEQ ID NO: 471 - protein, 116 aa, synthetic construct; identical to SEQ ID NO: 463
  source 1..116: note = D103388_1D7 [MICA/B]_H2L2 Variable heavy (vh) domain

SEQ ID NO: 472 - protein, 10 aa, synthetic construct
  source 1..10: note = D103388_1D7 [MICA/B]_H2L2 vhCDR1
GYSFTSYYIE

SEQ ID NO: 473 - protein, 17 aa, synthetic construct
  source 1..17: note = D103388_1D7 [MICA/B]_H2L2 vhCDR2
EIFPGNGDTNYNVGFTG

SEQ ID NO: 474 - protein, 7 aa, synthetic construct
  source 1..7: note = D103388_1D7 [MICA/B]_H2L2 vhCDR3
GHYNMDY

SEQ ID NO: 475 - protein, 107 aa, synthetic construct; identical to SEQ ID NO: 459
  source 1..107: note = D103388_1D7 [MICA/B]_H2L2 Variable light (vl) domain

SEQ ID NO: 476 - protein, 11 aa, synthetic construct
  source 1..11: note = D103388_1D7 [MICA/B]_H2L2 vlCDR1
KASQDISNYLN

SEQ ID NO: 477 - protein, 7 aa, synthetic construct
  source 1..7: note = D103388_1D7 [MICA/B]_H2L2 vlCDR2
YTSRLHS

SEQ ID NO: 478 - protein, 9 aa, synthetic construct
  source 1..9: note = D103388_1D7 [MICA/B]_H2L2 vlCDR3
QQGNTLPRT

SEQ ID NO: 479 - protein, 116 aa, synthetic construct
  source 1..116: note = D105317_1A2 [MICA/B]_H0L0 Variable heavy (vh) domain
QVQLKQSGPGLVQPSQSLSITCTVSGFSLTNNGVHWVRQSPGKGLEWLGVIWNVGITDYNAAFISRLSISKDNSKSQVFFKMNSLQGDDTAIYYCARKGGWAMDYWGQGTSVTVSS

SEQ ID NO: 480 - protein, 10 aa, synthetic construct
  source 1..10: note = D105317_1A2 [MICA/B]_H0L0 vhCDR1
GFSLTNNGVH

SEQ ID NO: 481 - protein, 16 aa, synthetic construct
  source 1..16: note = D105317_1A2 [MICA/B]_H0L0 vhCDR2
VIWNVGITDYNAAFIS

SEQ ID NO: 482 - protein, 8 aa, synthetic construct
  source 1..8: note = D105317_1A2 [MICA/B]_H0L0 vhCDR3
KGGWAMDY

SEQ ID NO: 483 - protein, 108 aa, synthetic construct
  source 1..108: note = D105317_1A2 [MICA/B]_H0L0 Variable light (vl) domain
QIALTQSPAIMFASLGERVTMTCTASSSVSSSYFHWFQQRPGSSPKLWIYSTSNLASGVPTRFSGSGSGTSYSLTISSMEAEDAATYYCHQYHRSPFTFGSGTKLEIK

SEQ ID NO: 484 - protein, 12 aa, synthetic construct
  source 1..12: note = D105317_1A2 [MICA/B]_H0L0 vlCDR1
TASSSVSSSYFH

SEQ ID NO: 485 - protein, 7 aa, synthetic construct
  source 1..7: note = D105317_1A2 [MICA/B]_H0L0 vlCDR2
STSNLAS

SEQ ID NO: 486 - protein, 9 aa, synthetic construct
  source 1..9: note = D105317_1A2 [MICA/B]_H0L0 vlCDR3
HQYHRSPFT

SEQ ID NO: 487 - protein, 116 aa, synthetic construct
  source 1..116: note = D105317_1A2 [MICA/B]_H1L1 Variable heavy (vh) domain
QVQLVQSGAEVKKPGASVKVSCKVSGFSLTNNGVHWVRQAPGQGLEWMGVIWNVGITDYNAKFQGRVTMTKDNSKSTVYMELSSLRSEDTAVYYCARKGGWAMDYWGQGTLVTVSS

SEQ ID NO: 488 - protein, 10 aa, synthetic construct
  source 1..10: note = D105317_1A2 [MICA/B]_H1L1 vhCDR1
GFSLTNNGVH

SEQ ID NO: 489 - protein, 16 aa, synthetic construct
  source 1..16: note = D105317_1A2 [MICA/B]_H1L1 vhCDR2
VIWNVGITDYNAKFQG

SEQ ID NO: 490 - protein, 8 aa, synthetic construct
  source 1..8: note = D105317_1A2 [MICA/B]_H1L1 vhCDR3
KGGWAMDY

SEQ ID NO: 491 - protein, 108 aa, synthetic construct
  source 1..108: note = D105317_1A2 [MICA/B]_H1L1 Variable light (vl) domain
QIALTQSPGTLSLSPGERATLSCRASSSVSSSYFHWFQQKPGQSPRLLIYSTSNLASGVPTRFSGSGSGTDYTLTISRLEPEDFAVYYCHQYHRSPFTFGSGTKLEIK

SEQ ID NO: 492 - protein, 12 aa, synthetic construct
  source 1..12: note = D105317_1A2 [MICA/B]_H1L1 vlCDR1
RASSSVSSSYFH

SEQ ID NO: 493 - protein, 7 aa, synthetic construct
  source 1..7: note = D105317_1A2 [MICA/B]_H1L1 vlCDR2
STSNLAS

SEQ ID NO: 494 - protein, 9 aa, synthetic construct
  source 1..9: note = D105317_1A2 [MICA/B]_H1L1 vlCDR3
HQYHRSPFT

SEQ ID NO: 495 - protein, 116 aa, synthetic construct; identical to SEQ ID NO: 487
  source 1..116: note = D105317_1A2 [MICA/B]_H1L2 Variable heavy (vh) domain

SEQ ID NO: 496 - protein, 10 aa, synthetic construct
  source 1..10: note = D105317_1A2 [MICA/B]_H1L2 vhCDR1
GFSLTNNGVH

SEQ ID NO: 497 - protein, 16 aa, synthetic construct
  source 1..16: note = D105317_1A2 [MICA/B]_H1L2 vhCDR2
VIWNVGITDYNAKFQG

SEQ ID NO: 498 - protein, 8 aa, synthetic construct
  source 1..8: note = D105317_1A2 [MICA/B]_H1L2 vhCDR3
KGGWAMDY

SEQ ID NO: 499 - protein, 108 aa, synthetic construct
  source 1..108: note = D105317_1A2 [MICA/B]_H1L2 Variable light (vl) domain
QIALTQSPFSLSASVGDRVTITCWASSSVSSSYFHWFQQKPAKSPKLFIYSTSNLASGVPTRFSGSGSGTDYTLTISSLQPEDFATYYCHQYHRSPFTFGSGTKLEIK

SEQ ID NO: 500 - protein, 12 aa, synthetic construct
  source 1..12: note = D105317_1A2 [MICA/B]_H1L2 vlCDR1
WASSSVSSSYFH

SEQ ID NO: 501 - protein, 7 aa, synthetic construct
  source 1..7: note = D105317_1A2 [MICA/B]_H1L2 vlCDR2
STSNLAS

SEQ ID NO: 502 - protein, 9 aa, synthetic construct
  source 1..9: note = D105317_1A2 [MICA/B]_H1L2 vlCDR3
HQYHRSPFT

SEQ ID NO: 503 - protein, 116 aa, synthetic construct
  source 1..116: note = D105317_1A2 [MICA/B]_H2L1 Variable heavy (vh) domain
EVQLVESGGGLVKPGGSLRLSCAVSGFSLTNNGVHWVRQAPGKGLEWVGVIWNVGITDYNAPVKGRFTISKDNSKSTVYLQMNSLKTEDTAVYYCARKGGWAMDYWGQGTLVTVSS

SEQ ID NO: 504 - protein, 10 aa, synthetic construct
  source 1..10: note = D105317_1A2 [MICA/B]_H2L1 vhCDR1
GFSLTNNGVH

SEQ ID NO: 505 - protein, 16 aa, synthetic construct
  source 1..16: note = D105317_1A2 [MICA/B]_H2L1 vhCDR2
VIWNVGITDYNAPVKG

SEQ ID NO: 506 - protein, 8 aa, synthetic construct
  source 1..8: note = D105317_1A2 [MICA/B]_H2L1 vhCDR3
KGGWAMDY

SEQ ID NO: 507 - protein, 108 aa, synthetic construct; identical to SEQ ID NO: 491
  source 1..108: note = D105317_1A2 [MICA/B]_H2L1 Variable light (vl) domain

SEQ ID NO: 508 - protein, 12 aa, synthetic construct
  source 1..12: note = D105317_1A2 [MICA/B]_H2L1 vlCDR1
RASSSVSSSYFH

SEQ ID NO: 509 - protein, 7 aa, synthetic construct
  source 1..7: note = D105317_1A2 [MICA/B]_H2L1 vlCDR2
STSNLAS

SEQ ID NO: 510 - protein, 9 aa, synthetic construct
  source 1..9: note = D105317_1A2 [MICA/B]_H2L1 vlCDR3
HQYHRSPFT

SEQ ID NO: 511 - protein, 116 aa, synthetic construct; identical to SEQ ID NO: 503
  source 1..116: note = D105317_1A2 [MICA/B]_H2L2 Variable heavy (vh) domain

SEQ ID NO: 512 - protein, 10 aa, synthetic construct
  source 1..10: note = D105317_1A2 [MICA/B]_H2L2 vhCDR1
GFSLTNNGVH

SEQ ID NO: 513 - protein, 16 aa, synthetic construct
  source 1..16: note = D105317_1A2 [MICA/B]_H2L2 vhCDR2
VIWNVGITDYNAPVKG

SEQ ID NO: 514 - protein, 8 aa, synthetic construct
  source 1..8: note = D105317_1A2 [MICA/B]_H2L2 vhCDR3
KGGWAMDY

SEQ ID NO: 515 - protein, 108 aa, synthetic construct; identical to SEQ ID NO: 499
  source 1..108: note = D105317_1A2 [MICA/B]_H2L2 Variable light (vl) domain

SEQ ID NO: 516 - protein, 12 aa, synthetic construct
  source 1..12: note = D105317_1A2 [MICA/B]_H2L2 vlCDR1
WASSSVSSSYFH

SEQ ID NO: 517 - protein, 7 aa, synthetic construct
  source 1..7: note = D105317_1A2 [MICA/B]_H2L2 vlCDR2
STSNLAS

SEQ ID NO: 518 - protein, 9 aa, synthetic construct
  source 1..9: note = D105317_1A2 [MICA/B]_H2L2 vlCDR3
HQYHRSPFT

SEQ ID NO: 519 - protein, 117 aa, synthetic construct
  source 1..117: note = D99136_2C11 [MICA/B]_H0L0 Variable heavy (vh) domain
DVQLQESGPGLVKPSQSLSLTCSVTGYSITSGYNWDWIRHSPGNKLEWMGYISYDGSTNYNPSLKNRISITRDTSKNQFFLKLNSVTTEDTATYYCASSYYRYFDYWGQGTTLTVSS

SEQ ID NO: 520 - protein, 11 aa, synthetic construct
  source 1..11: note = D99136_2C11 [MICA/B]_H0L0 vhCDR1
GYSITSGYNWD

SEQ ID NO: 521 - protein, 16 aa, synthetic construct
  source 1..16: note = D99136_2C11 [MICA/B]_H0L0 vhCDR2
YISYDGSTNYNPSLKN

SEQ ID NO: 522 - protein, 8 aa, synthetic construct
  source 1..8: note = D99136_2C11 [MICA/B]_H0L0 vhCDR3
SYYRYFDY

SEQ ID NO: 523 - protein, 107 aa, synthetic construct
  source 1..107: note = D99136_2C11 [MICA/B]_H0L0 Variable light (vl) domain
DVQMTQTTSSLSASLGDRVTISCSASQGISNYLNWFQQKPDGTVKLLIYYTSSLHSGVPSRFSGSGSGTDYSLTISNLEPEDIATYYCQQYTKLPWTFGGGTELEIK

SEQ ID NO: 524 - protein, 11 aa, synthetic construct
  source 1..11: note = D99136_2C11 [MICA/B]_H0L0 vlCDR1
SASQGISNYLN

SEQ ID NO: 525 - protein, 7 aa, synthetic construct
  source 1..7: note = D99136_2C11 [MICA/B]_H0L0 vlCDR2
YTSSLHS

SEQ ID NO: 526 - protein, 9 aa, synthetic construct
  source 1..9: note = D99136_2C11 [MICA/B]_H0L0 vlCDR3
QQYTKLPWT

SEQ ID NO: 527 - protein, 117 aa, synthetic construct
  source 1..117: note = D99136_2C11 [MICA/B]_H1L1 Variable heavy (vh) domain
QVQLQESGPGLVKPSETLSLTCTVSGYSITSGYNWDWIRHPPGKKLEWIGYISYDGSTNYNPSLKSRVTISRDTSKNQFSLKLSSVTAADTAVYYCASSYYRYFDYWGQGTLVTVSS

SEQ ID NO: 528 - protein, 11 aa, synthetic construct
  source 1..11: note = D99136_2C11 [MICA/B]_H1L1 vhCDR1
GYSITSGYNWD

SEQ ID NO: 529 - protein, 16 aa, synthetic construct
  source 1..16: note = D99136_2C11 [MICA/B]_H1L1 vhCDR2
YISYDGSTNYNPSLKS

SEQ ID NO: 530 - protein, 8 aa, synthetic construct
  source 1..8: note = D99136_2C11 [MICA/B]_H1L1 vhCDR3
SYYRYFDY

SEQ ID NO: 531 - protein, 107 aa, synthetic construct
  source 1..107: note = D99136_2C11 [MICA/B]_H1L1 Variable light (vl) domain
DVQMTQSPSSLSASVGDRVTITCRASQGISNYLNWFQQKPGKTVKLLIYYTSSLHSGVPSRFSGSGSGTDYTLTISSLQPEDFATYYCQQYTKLPWTFGGGTKVEIK

SEQ ID NO: 532 - protein, 11 aa, synthetic construct
  source 1..11: note = D99136_2C11 [MICA/B]_H1L1 vlCDR1
RASQGISNYLN

SEQ ID NO: 533 - protein, 7 aa, synthetic construct
  source 1..7: note = D99136_2C11 [MICA/B]_H1L1 vlCDR2
YTSSLHS

SEQ ID NO: 534 - protein, 9 aa, synthetic construct
  source 1..9: note = D99136_2C11 [MICA/B]_H1L1 vlCDR3
QQYTKLPWT

SEQ ID NO: 535 - protein, 117 aa, synthetic construct; identical to SEQ ID NO: 527
  source 1..117: note = D99136_2C11 [MICA/B]_H1L2 Variable heavy (vh) domain

SEQ ID NO: 536 - protein, 11 aa, synthetic construct
  source 1..11: note = D99136_2C11 [MICA/B]_H1L2 vhCDR1
GYSITSGYNWD

SEQ ID NO: 537 - protein, 16 aa, synthetic construct
  source 1..16: note = D99136_2C11 [MICA/B]_H1L2 vhCDR2
YISYDGSTNYNPSLKS

SEQ ID NO: 538 - protein, 8 aa, synthetic construct
  source 1..8: note = D99136_2C11 [MICA/B]_H1L2 vhCDR3
SYYRYFDY

SEQ ID NO: 539 - protein, 107 aa, synthetic construct
  source 1..107: note = D99136_2C11 [MICA/B]_H1L2 Variable light (vl) domain
DVQMTQSPLSLPVTLGQPASISCRASQGISNYLNWFQQRPGQTVRLLIYYTSSLHSGVPSRFSGSGSGTDYTLKISRVEAEDVGVYYCQQYTKLPWTFGGGTKVEIK

SEQ ID NO: 540 - protein, 11 aa, synthetic construct
  source 1..11: note = D99136_2C11 [MICA/B]_H1L2 vlCDR1
RASQGISNYLN

SEQ ID NO: 541 - protein, 7 aa, synthetic construct
  source 1..7: note = D99136_2C11 [MICA/B]_H1L2 vlCDR2
YTSSLHS

SEQ ID NO: 542 - protein, 9 aa, synthetic construct
  source 1..9: note = D99136_2C11 [MICA/B]_H1L2 vlCDR3
QQYTKLPWT

SEQ ID NO: 543 - protein, 117 aa, synthetic construct
  source 1..117: note = D99136_2C11 [MICA/B]_H2L1 Variable heavy (vh) domain
QVQLVQSGAEVKKPGASVKVSCKVSGYSITSGYNWDWIRHAPGQKLEWMGYISYDGSTNYNPKFQGRVTMTRDTSINTFYMELSRLRSDDTAVYYCASSYYRYFDYWGQGTLVTVSS

SEQ ID NO: 544 - protein, 11 aa, synthetic construct
  source 1..11: note = D99136_2C11 [MICA/B]_H2L1 vhCDR1
GYSITSGYNWD

SEQ ID NO: 545 - protein, 16 aa, synthetic construct
  source 1..16: note = D99136_2C11 [MICA/B]_H2L1 vhCDR2
YISYDGSTNYNPKFQG

SEQ ID NO: 546 - protein, 8 aa, synthetic construct
  source 1..8: note = D99136_2C11 [MICA/B]_H2L1 vhCDR3
SYYRYFDY

SEQ ID NO: 547 - protein, 107 aa, synthetic construct; identical to SEQ ID NO: 531
  source 1..107: note = D99136_2C11 [MICA/B]_H2L1 Variable light (vl) domain

SEQ ID NO: 548 - protein, 11 aa, synthetic construct
  source 1..11: note = D99136_2C11 [MICA/B]_H2L1 vlCDR1
RASQGISNYLN

SEQ ID NO: 549 - protein, 7 aa, synthetic construct
  source 1..7: note = D99136_2C11 [MICA/B]_H2L1 vlCDR2
YTSSLHS

SEQ ID NO: 550 - protein, 9 aa, synthetic construct
  source 1..9: note = D99136_2C11 [MICA/B]_H2L1 vlCDR3
QQYTKLPWT

SEQ ID NO: 551 - protein, 117 aa, synthetic construct; identical to SEQ ID NO: 543
  source 1..117: note = D99136_2C11 [MICA/B]_H2L2 Variable heavy (vh) domain

SEQ ID NO: 552 - protein, 11 aa, synthetic construct
  source 1..11: note = D99136_2C11 [MICA/B]_H2L2 vhCDR1
GYSITSGYNWD

SEQ ID NO: 553 - protein, 16 aa, synthetic construct
  source 1..16: note = D99136_2C11 [MICA/B]_H2L2 vhCDR2
YISYDGSTNYNPKFQG

SEQ ID NO: 554 - protein, 8 aa, synthetic construct
  source 1..8: note = D99136_2C11 [MICA/B]_H2L2 vhCDR3
SYYRYFDY

SEQ ID NO: 555 - protein, 107 aa, synthetic construct; identical to SEQ ID NO: 539
  source 1..107: note = D99136_2C11 [MICA/B]_H2L2 Variable light (vl) domain

SEQ ID NO: 556 - protein, 11 aa, synthetic construct
  source 1..11: note = D99136_2C11 [MICA/B]_H2L2 vlCDR1
RASQGISNYLN

SEQ ID NO: 557 - protein, 7 aa, synthetic construct
  source 1..7: note = D99136_2C11 [MICA/B]_H2L2 vlCDR2
YTSSLHS

SEQ ID NO: 558 - protein, 9 aa, synthetic construct
  source 1..9: note = D99136_2C11 [MICA/B]_H2L2 vlCDR3
QQYTKLPWT

SEQ ID NO: 559 - protein, 116 aa, synthetic construct
  source 1..116: note = D103388_1B11-2 [MICA/B]_H0L0 Variable heavy (vh) domain
QVHLNQSGPGLVQPSQSLSITCTVSGFSLTNYGVHWIRQSPGKGLEWLGVIWNVGITDYNAAFVSRLSITKDNSKSQVFFKLNSLQTIDTAIYYCARKGGIAMDYWGQGTSVTVSS

SEQ ID NO: 560 - protein, 10 aa, synthetic construct
  source 1..10: note = D103388_1B11-2 [MICA/B]_H0L0 vhCDR1
GFSLTNYGVH

SEQ ID NO: 561 - protein, 16 aa, synthetic construct
  source 1..16: note = D103388_1B11-2 [MICA/B]_H0L0 vhCDR2
VIWNVGITDYNAAFVS

SEQ ID NO: 562 - protein, 8 aa, synthetic construct
  source 1..8: note = D103388_1B11-2 [MICA/B]_H0L0 vhCDR3
KGGIAMDY

SEQ ID NO: 563 - protein, 108 aa, synthetic construct
  source 1..108: note = D103388_1B11-2 [MICA/B]_H0L0 Variable light (vl) domain
QIVLTQSPAIMSASLGDRVTMTCTASSSVSSTYLHWYQQKPGSSPKLWIYSTSNLASGVPARFSGSGSGTSYSLTISSLEAEDSTTYYCHQYHRSPFTFGSGTKLEIK

SEQ ID NO: 564 - protein, 12 aa, synthetic construct
  source 1..12: note = D103388_1B11-2 [MICA/B]_H0L0 vlCDR1
TASSSVSSTYLH

SEQ ID NO: 565 - protein, 7 aa, synthetic construct
  source 1..7: note = D103388_1B11-2 [MICA/B]_H0L0 vlCDR2
STSNLAS

SEQ ID NO: 566 - protein, 9 aa, synthetic construct
  source 1..9: note = D103388_1B11-2 [MICA/B]_H0L0 vlCDR3
HQYHRSPFT

SEQ ID NO: 567 - protein, 116 aa, synthetic construct
  source 1..116: note = D105317_1B6 [MICA/B]_H0L0 Variable heavy (vh) domain
QVQLKQSGPGLVQPSQSLSLTCTVSGFSLSNFGVHWVRQSPGKGLEWLGVIWNVGITDFNAAFISRLSISKDNSKSQVFFKMNSLQVDDTAIYYCARKGPYAMDYWGQGTSVTVSS

SEQ ID NO: 568 - protein, 10 aa, synthetic construct
  source 1..10: note = D105317_1B6 [MICA/B]_H0L0 vhCDR1
GFSLSNFGVH

SEQ ID NO: 569 - protein, 16 aa, synthetic construct
  source 1..16: note = D105317_1B6 [MICA/B]_H0L0 vhCDR2
VIWNVGITDFNAAFIS

SEQ ID NO: 570 - protein, 8 aa, synthetic construct
  source 1..8: note = D105317_1B6 [MICA/B]_H0L0 vhCDR3
KGPYAMDY

SEQ ID NO: 571 - protein, 108 aa, synthetic construct
  source 1..108: note = D105317_1B6 [MICA/B]_H0L0 Variable light (vl) domain
QIVLTQSPAIMSASLGERVTMTCTASSSVSSSYLHWYQQKPGSSPKLWISSTSNLASGVPGRFSGSGSGTSYSLTISSMEAEDAATYYCHQYHRSPFTFGSGTKLEIK

SEQ ID NO: 572 - protein, 12 aa, synthetic construct
  source 1..12: note = D105317_1B6 [MICA/B]_H0L0 vlCDR1
TASSSVSSSYLH

SEQ ID NO: 573 - protein, 7 aa, synthetic construct
  source 1..7: note = D105317_1B6 [MICA/B]_H0L0 vlCDR2
STSNLAS

SEQ ID NO: 574 - protein, 9 aa, synthetic construct
  source 1..9: note = D105317_1B6 [MICA/B]_H0L0 vlCDR3
HQYHRSPFT

SEQ ID NO: 575 - protein, 116 aa, synthetic construct
  source 1..116: note = D105317_1C8 [MICA/B]_H0L0 Variable heavy (vh) domain
QVQLKQSGPGLVQPSQSLSFTCTVSGFSLTNYGVHWVRQSPGKGLEWLGVIWNSGTTDYNAAFISRLSINKDNSKSQVFFKMNSLQADDTAIYYCARKGSYAMDYWGQGTSVTVSS

SEQ ID NO: 576 - protein, 10 aa, synthetic construct
  source 1..10: note = D105317_1C8 [MICA/B]_H0L0 vhCDR1
GFSLTNYGVH

SEQ ID NO: 577 - protein, 16 aa, synthetic construct
  source 1..16: note = D105317_1C8 [MICA/B]_H0L0 vhCDR2
VIWNSGTTDYNAAFIS

SEQ ID NO: 578 - protein, 8 aa, synthetic construct
  source 1..8: note = D105317_1C8 [MICA/B]_H0L0 vhCDR3
KGSYAMDY

SEQ ID NO: 579 - protein, 108 aa, synthetic construct
  source 1..108: note = D105317_1C8 [MICA/B]_H0L0 Variable light (vl) domain
QIVLTQSPAIMSASLGERVTMTCTASSSVSSIYLHWYQQKPGSSPKLWISSTSNLASGVPARFNGSGSGTSYSLTVSNMEAEDAATYYCHQYHRSPFTFGSGTKLDIK

SEQ ID NO: 580 - protein, 12 aa, synthetic construct
  source 1..12: note = D105317_1C8 [MICA/B]_H0L0 vlCDR1
TASSSVSSIYLH

SEQ ID NO: 581 - protein, 7 aa, synthetic construct
  source 1..7: note = D105317_1C8 [MICA/B]_H0L0 vlCDR2
STSNLAS

SEQ ID NO: 582 - protein, 9 aa, synthetic construct
  source 1..9: note = D105317_1C8 [MICA/B]_H0L0 vlCDR3
HQYHRSPFT

SEQ ID NO: 583 - protein, 116 aa, synthetic construct
  source 1..116: note = D105317_1F6 [MICA/B]_H0L0 Variable heavy (vh) domain
QVQLKQSGPGLVQPSQSLSITCTVSGFSLTNYGVHWVRQSPGKGLEWLGVIWSGGITDYNAAFISRLSISKDNSKSQVFFKMNSLQADDTAIYYCARRGGYAMDYWGQGTSVTVSS

SEQ ID NO: 584 - protein, 10 aa, synthetic construct
  source 1..10: note = D105317_1F6 [MICA/B]_H0L0 vhCDR1
GFSLTNYGVH

SEQ ID NO: 585 - protein, 16 aa, synthetic construct
  source 1..16: note = D105317_1F6 [MICA/B]_H0L0 vhCDR2
VIWSGGITDYNAAFIS

SEQ ID NO: 586 - protein, 8 aa, synthetic construct
  source 1..8: note = D105317_1F6 [MICA/B]_H0L0 vhCDR3
RGGYAMDY

SEQ ID NO: 587 - protein, 108 aa, synthetic construct
  source 1..108: note = D105317_1F6 [MICA/B]_H0L0 Variable light (vl) domain
QIVLTQSPAIMSASLGERVTMTCTASSSVSSSYLHWYQQKPGSSPKLWIYSTSNLASGVPARFSGSGSGTSYSLTINTMEAEDAATYYCHQYHRSPFTFGSGTKLEIK

SEQ ID NO: 588 - protein, 12 aa, synthetic construct
  source 1..12: note = D105317_1F6 [MICA/B]_H0L0 vlCDR1
TASSSVSSSYLH

SEQ ID NO: 589 - protein, 7 aa, synthetic construct
  source 1..7: note = D105317_1F6 [MICA/B]_H0L0 vlCDR2
STSNLAS

SEQ ID NO: 590 - protein, 9 aa, synthetic construct
  source 1..9: note = D105317_1F6 [MICA/B]_H0L0 vlCDR3
HQYHRSPFT

SEQ ID NO: 591 - protein, 118 aa, synthetic construct
  source 1..118: note = D105317_1F7 [MICA/B]_H0L0 Variable heavy (vh) domain
DIQLQESGPGLVKPSQSLSLTCSVTGYSITSGYYWNWIRQFPGNKLEWMGYIRYDGRNNYNPSLKNRISITRDTSKNQFFLRLNSVTTEDTATYYCASSFHYYAMDYWGQGTSVTVSS

SEQ ID NO: 592 - protein, 11 aa, synthetic construct
  source 1..11: note = D105317_1F7 [MICA/B]_H0L0 vhCDR1
GYSITSGYYWN

SEQ ID NO: 593 - protein, 16 aa, synthetic construct
  source 1..16: note = D105317_1F7 [MICA/B]_H0L0 vhCDR2
YIRYDGRNNYNPSLKN

SEQ ID NO: 594 - protein, 9 aa, synthetic construct
  source 1..9: note = D105317_1F7 [MICA/B]_H0L0 vhCDR3
SFHYYAMDY

SEQ ID NO: 595 - protein, 111 aa, synthetic construct
  source 1..111: note = D105317_1F7 [MICA/B]_H0L0 Variable light (vl) domain
DIVLTQSPGSLSVSPGQRATISCRASESVDSYGDSFLHWYQQKPGQSPKLLIYRASNLESGIPARFSGSGSRTDFTLTINPIEADDVATYYCQQSNEDPWTFGGGTNLEIK

SEQ ID NO: 596 - protein, 15 aa, synthetic construct
  source 1..15: note = D105317_1F7 [MICA/B]_H0L0 vlCDR1
RASESVDSYGDSFLH

SEQ ID NO: 597 - protein, 7 aa, synthetic construct
  source 1..7: note = D105317_1F7 [MICA/B]_H0L0 vlCDR2
RASNLES

SEQ ID NO: 598 - protein, 9 aa, synthetic construct
  source 1..9: note = D105317_1F7 [MICA/B]_H0L0 vlCDR3
QQSNEDPWT

SEQ ID NO: 599 - protein, 116 aa, synthetic construct
  source 1..116: note = D94837_1D3 [MICA/B]_H0L0 Variable heavy (vh) domain
QVQLRQSGPGLGQPSQSLSITCTVSGFSLTNHGVHWVRQSLGKGLEWLGVIWNNGITDYNADFISRLSITKDNSKSQVFFKMSSLQANDTAIYYCARKGSYAMDYWGQGSSVTVSS

SEQ ID NO: 600 - protein, 10 aa, synthetic construct
  source 1..10: note = D94837_1D3 [MICA/B]_H0L0 vhCDR1
GFSLTNHGVH

SEQ ID NO: 601 - protein, 16 aa, synthetic construct
  source 1..16: note = D94837_1D3 [MICA/B]_H0L0 vhCDR2
VIWNNGITDYNADFIS

SEQ ID NO: 602 - protein, 8 aa, synthetic construct
  source 1..8: note = D94837_1D3 [MICA/B]_H0L0 vhCDR3
KGSYAMDY

SEQ ID NO: 603 - protein, 108 aa, synthetic construct
  source 1..108: note = D94837_1D3 [MICA/B]_H0L0 Variable light (vl) domain
QIVLTQSPAIMSASLGERVTMTCTASSSVSSSYFHWYQQKPGSSPKLWIYSTSNLASGVPARFSGSGSGTSYSLTISSMEAEDAATYYCHQYHRSPWTFGGGTKLEIK

SEQ ID NO: 604 - protein, 12 aa, synthetic construct
  source 1..12: note = D94837_1D3 [MICA/B]_H0L0 vlCDR1
TASSSVSSSYFH

SEQ ID NO: 605 - protein, 7 aa, synthetic construct
  source 1..7: note = D94837_1D3 [MICA/B]_H0L0 vlCDR2
STSNLAS

SEQ ID NO: 606 - protein, 9 aa, synthetic construct
  source 1..9: note = D94837_1D3 [MICA/B]_H0L0 vlCDR3
HQYHRSPWT

SEQ ID NO: 607 - protein, 118 aa, synthetic construct
  source 1..118: note = D94837_1D8 [MICA/B]_H0L0 Variable heavy (vh) domain
QVQLKQSGPGLVQPSQSLSITCTVFGFSLTNYGVHWVRQSPGKGLEWLGVIWSDGYTDYYAAFVSRLSISKDNSKSQVFFKMNSLQADDTAIYYCARKGGDPYAMDYWGQGTSVTVSS

SEQ ID NO: 608 - protein, 10 aa, synthetic construct
  source 1..10: note = D94837_1D8 [MICA/B]_H0L0 vhCDR1
GFSLTNYGVH

SEQ ID NO: 609 - protein, 16 aa, synthetic construct
  source 1..16: note = D94837_1D8 [MICA/B]_H0L0 vhCDR2
VIWSDGYTDYYAAFVS

SEQ ID NO: 610 - protein, 10 aa, synthetic construct
  source 1..10: note = D94837_1D8 [MICA/B]_H0L0 vhCDR3
KGGDPYAMDY

SEQ ID NO: 611 - protein, 108 aa, synthetic construct
  source 1..108: note = D94837_1D8 [MICA/B]_H0L0 Variable light (vl) domain
QIVLTQSPAIMSASLGERVTMTCTASSSVSSSYLHWYQQKPGSSPKLWIFSTSNLASGVPARFSGSGSGTSYSLTISSMEAEDAATYYCHQYHRSPFTFGSGTKLEIK

SEQ ID NO: 612 - protein, 12 aa, synthetic construct
  source 1..12: note = D94837_1D8 [MICA/B]_H0L0 vlCDR1
TASSSVSSSYLH

SEQ ID NO: 613 - protein, 7 aa, synthetic construct
  source 1..7: note = D94837_1D8 [MICA/B]_H0L0 vlCDR2
STSNLAS

SEQ ID NO: 614 - protein, 9 aa, synthetic construct
  source 1..9: note = D94837_1D8 [MICA/B]_H0L0 vlCDR3
HQYHRSPFT

SEQ ID NO: 615 - protein, 119 aa, synthetic construct
  source 1..119: note = D94837_1D9 [MICA/B]_H0L0 Variable heavy (vh) domain
QVQLQQSGAELVRPGTSVKMSCKAAGYTFTNYWIGWVKQRPGHGLEWIGDIYPGGDYTNYNGKFKARATLTADTSASTAYMHLSSLTSEDSAIYYCARRNHYPYVMDDWGQGTSVTVSS

SEQ ID NO: 616 - protein, 10 aa, synthetic construct
  source 1..10: note = D94837_1D9 [MICA/B]_H0L0 vhCDR1
GYTFTNYWIG

SEQ ID NO: 617 - protein, 17 aa, synthetic construct
  source 1..17: note = D94837_1D9 [MICA/B]_H0L0 vhCDR2
DIYPGGDYTNYNGKFKA

SEQ ID NO: 618 - protein, 10 aa, synthetic construct
  source 1..10: note = D94837_1D9 [MICA/B]_H0L0 vhCDR3
RNHYPYVMDD

SEQ ID NO: 619 - protein, 108 aa, synthetic construct
  source 1..108: note = D94837_1D9 [MICA/B]_H0L0 Variable light (vl) domain
QIVLTQSPAIMSASLGERVTMTCTASSSVSSSYLHWYQQKPGSSPKLWIYSTSNLASGVPPRFSGSGSGTSYSLTISSMEAEDAATYYCHQYHRSPWTFGGGTKLEIK

SEQ ID NO: 620 - protein, 12 aa, synthetic construct
  source 1..12: note = D94837_1D9 [MICA/B]_H0L0 vlCDR1
TASSSVSSSYLH

SEQ ID NO: 621 - protein, 7 aa, synthetic construct
  source 1..7: note = D94837_1D9 [MICA/B]_H0L0 vlCDR2
STSNLAS

SEQ ID NO: 622 - protein, 9 aa, synthetic construct
  source 1..9: note = D94837_1D9 [MICA/B]_H0L0 vlCDR3
HQYHRSPWT

SEQ ID NO: 623 - protein, 116 aa, synthetic construct
  source 1..116: note = D94837_1E1 [MICA/B]_H0L0 Variable heavy (vh) domain
QVQLKQSGPGLVQPSQSLSITCTVSGFSLTNYGVHWVRQSPGKGLEWLGVIWNVGITDYNAAFISRLSISKDNSKSQVFLKMNSLQADDTAIYYCARKGSYAMDYWGQGTSVTVSS

SEQ ID NO: 624 - protein, 10 aa, synthetic construct
  source 1..10: note = D94837_1E1 [MICA/B]_H0L0 vhCDR1
GFSLTNYGVH

SEQ ID NO: 625 - protein, 16 aa, synthetic construct
  source 1..16: note = D94837_1E1 [MICA/B]_H0L0 vhCDR2
VIWNVGITDYNAAFIS

SEQ ID NO: 626 - protein, 8 aa, synthetic construct
  source 1..8: note = D94837_1E1 [MICA/B]_H0L0 vhCDR3
KGSYAMDY

SEQ ID NO: 627 - protein, 108 aa, synthetic construct
  source 1..108: note = D94837_1E1 [MICA/B]_H0L0 Variable light (vl) domain
QIVLTQSPAIMSASLGERVTMTCTASSSVSSSYLHWYHQKPGSSPTLWIYSTSNLASGVPTRFSGSGSGTSYSLTISSMEAEDAATYYCHQYHRSPFTFGSGTKLEIK

SEQ ID NO: 628 - protein, 12 aa, synthetic construct
  source 1..12: note = D94837_1E1 [MICA/B]_H0L0 vlCDR1
TASSSVSSSYLH

SEQ ID NO: 629 - protein, 7 aa, synthetic construct
  source 1..7: note = D94837_1E1 [MICA/B]_H0L0 vlCDR2
STSNLAS

SEQ ID NO: 630 - protein, 9 aa, synthetic construct
  source 1..9: note = D94837_1E1 [MICA/B]_H0L0 vlCDR3
HQYHRSPFT

SEQ ID NO: 631 - protein, 117 aa, synthetic construct
  source 1..117: note = D94852_2D4 [MICA/B]_H0L0 Variable heavy (vh) domain
QVQLKESGPGLVAPSQSLSITCTVSGFSLTSYGVHWVRQPPGKGLEWLGVIWTGGNTNYNSAFMSRVSISKDNSKSQVFLKMNSLQTDDTAMYYCARFQYDYDVAYWGQGTLVTVSA

SEQ ID NO: 632 - protein, 10 aa, synthetic construct
  source 1..10: note = D94852_2D4 [MICA/B]_H0L0 vhCDR1
GFSLTSYGVH

SEQ ID NO: 633 - protein, 16 aa, synthetic construct
  source 1..16: note = D94852_2D4 [MICA/B]_H0L0 vhCDR2
VIWTGGNTNYNSAFMS

SEQ ID NO: 634 - protein, 9 aa, synthetic construct
  source 1..9: note = D94852_2D4 [MICA/B]_H0L0 vhCDR3
FQYDYDVAY

SEQ ID NO: 635 - protein, 108 aa, synthetic construct
  source 1..108: note = D94852_2D4 [MICA/B]_H0L0 Variable light (vl) domain
QIVLTQSPAIMSASLGERVTVTCTASSSVSSSFLHWYQQKPGSSPKLWIYSTSNLASGVPARFSGSGSGTSYSLTISSMEAEDAATYYCHQYHRSPFTFGSGTKLEIK

SEQ ID NO: 636 - protein, 12 aa, synthetic construct
  source 1..12: note = D94852_2D4 [MICA/B]_H0L0 vlCDR1
TASSSVSSSFLH

SEQ ID NO: 637 - protein, 7 aa, synthetic construct
  source 1..7: note = D94852_2D4 [MICA/B]_H0L0 vlCDR2
STSNLAS

SEQ ID NO: 638 - protein, 9 aa, synthetic construct
  source 1..9: note = D94852_2D4 [MICA/B]_H0L0 vlCDR3
HQYHRSPFT

SEQ ID NO: 639 - protein, 117 aa, synthetic construct
  source 1..117: note = D94852_2G8 [MICA/B]_H0L0 Variable heavy (vh) domain
QVQLKQSGPGLVQPSQSLSITCTVSGFSLTNYGVHWVRQSPGKGLEWLGVIWSGGNTDYNAAFISRLSISKDNSKSQVFFKMNSLQPNDTALFYCARGTYTYGFDYWGQGTLVTVSA

SEQ ID NO: 640 - protein, 10 aa, synthetic construct
  source 1..10: note = D94852_2G8 [MICA/B]_H0L0 vhCDR1
GFSLTNYGVH

SEQ ID NO: 641 - protein, 16 aa, synthetic construct
  source 1..16: note = D94852_2G8 [MICA/B]_H0L0 vhCDR2
VIWSGGNTDYNAAFIS

SEQ ID NO: 642 - protein, 9 aa, synthetic construct
  source 1..9: note = D94852_2G8 [MICA/B]_H0L0 vhCDR3
GTYTYGFDY

SEQ ID NO: 643 - protein, 108 aa, synthetic construct
  source 1..108: note = D94852_2G8 [MICA/B]_H0L0 Variable light (vl) domain
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPWTFGGGTKLEIK

SEQ ID NO: 644 - protein, 12 aa, synthetic construct
  source 1..12: note = D94852_2G8 [MICA/B]_H0L0 vlCDR1
TASSSVSSSYLH

SEQ ID NO: 645 - protein, 7 aa, synthetic construct
  source 1..7: note = D94852_2G8 [MICA/B]_H0L0 vlCDR2
STSNLAS

SEQ ID NO: 646 - protein, 9 aa, synthetic construct
  source 1..9: note = D94852_2G8 [MICA/B]_H0L0 vlCDR3
HQYHRSPWT

SEQ ID NO: 647 - protein, 118 aa, synthetic construct
  source 1..118: note = D88487_2A8 [MICA/B]_H0L0 Variable heavy (vh) domain
QVQLKQSGPGLVQPSQSLSITCTVSGFSLIKYGVHWVRQFPGKGLEWLGVIWNEGYTDYYAAFISRLSISKDNSKSQVFFKMNSLQANDTAIYYCARKGGDPYAMDYWGQGTSVTVSS

SEQ ID NO: 648 - protein, 10 aa, synthetic construct
  source 1..10: note = D88487_2A8 [MICA/B]_H0L0 vhCDR1
GFSLIKYGVH

SEQ ID NO: 649 - protein, 16 aa, synthetic construct
  source 1..16: note = D88487_2A8 [MICA/B]_H0L0 vhCDR2
VIWNEGYTDYYAAFIS

SEQ ID NO: 650 - protein, 10 aa, synthetic construct
  source 1..10: note = D88487_2A8 [MICA/B]_H0L0 vhCDR3
KGGDPYAMDY

SEQ ID NO: 651 - protein, 108 aa, synthetic construct
  source 1..108: note = D88487_2A8 [MICA/B]_H0L0 Variable light (vl) domain
QIVLTQSPAIMSASLGERVTMTCTASSSVSSSYLHWFQQKPGSSPKLWIYSTSNLASGVPARFSGSGSGTSYSLTISSMEAEDAVTYYCHQYHRSPFTFGSGTKLEIK

SEQ ID NO: 652 - protein, 12 aa, synthetic construct
  source 1..12: note = D88487_2A8 [MICA/B]_H0L0 vlCDR1
TASSSVSSSYLH

SEQ ID NO: 653 - protein, 7 aa, synthetic construct
  source 1..7: note = D88487_2A8 [MICA/B]_H0L0 vlCDR2
STSNLAS

SEQ ID NO: 654 - protein, 9 aa, synthetic construct
  source 1..9: note = D88487_2A8 [MICA/B]_H0L0 vlCDR3
HQYHRSPFT

SEQ ID NO: 655 - protein, 117 aa, synthetic construct
  source 1..117: note = D94837_3B12 [MICA/B]_H0L0 Variable heavy (vh) domain
QVQLQQSGAELAKPGASVKMSCKASGYTFTSYWMHWVKQRPGQGLEWIGYINPNTGYTEYNQKFKDKATLTADRSSSTAYMQLSSLTSEDSAVYYCARTYDYDGAYWGQGTLVTVSS

SEQ ID NO: 656 - protein, 10 aa, synthetic construct
  source 1..10: note = D94837_3B12 [MICA/B]_H0L0 vhCDR1
GYTFTSYWMH

SEQ ID NO: 657 - protein, 17 aa, synthetic construct
  source 1..17: note = D94837_3B12 [MICA/B]_H0L0 vhCDR2
YINPNTGYTEYNQKFKD

SEQ ID NO: 658 - protein, 8 aa, synthetic construct
  source 1..8: note = D94837_3B12 [MICA/B]_H0L0 vhCDR3
TYDYDGAY

SEQ ID NO: 659 - protein, 106 aa, synthetic construct
  source 1..106: note = D94837_3B12 [MICA/B]_H0L0 Variable light (vl) domain
QIVLTQSPAIMSASPGESVTMTCSASSSVSYMSWYQQKPGSSPRLLIYGTSNLASGVPVRFSGSGSGTSYSLTISRMEAEDAATYYCQQWSSYPLTFGAGTKLELK

SEQ ID NO: 660 - protein, 10 aa, synthetic construct
  source 1..10: note = D94837_3B12 [MICA/B]_H0L0 vlCDR1
SASSSVSYMS

SEQ ID NO: 661 - protein, 7 aa, synthetic construct
  source 1..7: note = D94837_3B12 [MICA/B]_H0L0 vlCDR2
GTSNLAS

SEQ ID NO: 662 - protein, 9 aa, synthetic construct
  source 1..9: note = D94837_3B12 [MICA/B]_H0L0 vlCDR3
QQWSSYPLT

SEQ ID NO: 663 - protein, 116 aa, synthetic construct
  source 1..116: note = D99122_1H7 [MICA/B]_H0L0 Variable heavy (vh) domain
QVQLQQSGAELARPGASVKLSCKASGYTFISYGISWVKQRTGQGLEWIGEIYPRSGNTYYNEKFKGKATLTADKSSSTAYMEVRSLTSEDSAVYFCASNWDEKVYWGQGTTLTVSS

SEQ ID NO: 664 - protein, 10 aa, synthetic construct
  source 1..10: note = D99122_1H7 [MICA/B]_H0L0 vhCDR1
GYTFISYGIS

SEQ ID NO: 665 - protein, 17 aa, synthetic construct
  source 1..17: note = D99122_1H7 [MICA/B]_H0L0 vhCDR2
EIYPRSGNTYYNEKFKG

SEQ ID NO: 666 - protein, 7 aa, synthetic construct
  source 1..7: note = D99122_1H7 [MICA/B]_H0L0 vhCDR3
NWDEKVY

SEQ ID NO: 667 - protein, 111 aa, synthetic construct
  source 1..111: note = D99122_1H7 [MICA/B]_H0L0 Variable light (vl) domain
DIVLTQSPASLAVSLGQRATISCRASKSVDNYGISFMHWFQQKPGQPPKLLIYAASNQGSGVPARFSGSGSGTDFSLNIHPLEGDDTAMYFCQQSREFPWTFGGGTKLEIR

SEQ ID NO: 668 - protein, 15 aa, synthetic construct
  source 1..15: note = D99122_1H7 [MICA/B]_H0L0 vlCDR1
RASKSVDNYGISFMH

SEQ ID NO: 669 - protein, 7 aa, synthetic construct
  source 1..7: note = D99122_1H7 [MICA/B]_H0L0 vlCDR2
AASNQGS

SEQ ID NO: 670 - protein, 9 aa, synthetic construct
  source 1..9: note = D99122_1H7 [MICA/B]_H0L0 vlCDR3
QQSREFPWT

SEQ ID NO: 671 - protein, 116 aa, synthetic construct
  source 1..116: note = D99136_2E8 [MICA/B]_H0L0 Variable heavy (vh) domain
QVHLQQSGAELARPGASVKLSCKASGYTFISYGISWVKQGTGQGLEWIGEIYPRSGTTYYNEKFKVKATLTADKSSSTAYLELRSLTSEDSAVYFCASNWDEKVYWGQGTTLTVSS

SEQ ID NO: 672 - protein, 10 aa, synthetic construct
  source 1..10: note = D99136_2E8 [MICA/B]_H0L0 vhCDR1
GYTFISYGIS

SEQ ID NO: 673 - protein, 17 aa, synthetic construct
  source 1..17: note = D99136_2E8 [MICA/B]_H0L0 vhCDR2
EIYPRSGTTYYNEKFKV

SEQ ID NO: 674 - protein, 7 aa, synthetic construct
  source 1..7: note = D99136_2E8 [MICA/B]_H0L0 vhCDR3
NWDEKVY

SEQ ID NO: 675 - protein, 111 aa, synthetic construct
  source 1..111: note = D99136_2E8 [MICA/B]_H0L0 Variable light (vl) domain
DIVLTQSPASLAVSLGQRATISCRASESVDNYGISFMHWFQQKPGQPPKLLIYAASNQGSGVPARFSGSGSGTDFSLNIHPLEEDDTAMYFCQQSRKLPWTFGGGTKLEIR

SEQ ID NO: 676 - protein, 15 aa, synthetic construct
  source 1..15: note = D99136_2E8 [MICA/B]_H0L0 vlCDR1
RASESVDNYGISFMH

SEQ ID NO: 677 - protein, 7 aa, synthetic construct
  source 1..7: note = D99136_2E8 [MICA/B]_H0L0 vlCDR2
AASNQGS

SEQ ID NO: 678 - protein, 9 aa, synthetic construct
  source 1..9: note = D99136_2E8 [MICA/B]_H0L0 vlCDR3
QQSRKLPWT

SEQ ID NO: 679 - protein, 121 aa, synthetic construct
  source 1..121: note = 3F9[MICA/B]_H0L0 Variable heavy (vh) domain
EVQLQESGEGLVKPGGSLKLSCAASGFTFSNYAMSWVRQTPEKRLEWVAYISPGGDYIYYADTVKGRFTISRDNARNTLYLQMSSLKSEDTAMYYCTTDRRHYGSYAMDYWGQGISVTVSS

SEQ ID NO: 680 - protein, 5 aa, synthetic construct
  source 1..5: note = 3F9[MICA/B]_H0L0 vhCDR1
NYAMS

SEQ ID NO: 681 - protein, 17 aa, synthetic construct
  source 1..17: note = 3F9[MICA/B]_H0L0 vhCDR2
YISPGGDYIYYADTVKG

SEQ ID NO: 682 - protein, 12 aa, synthetic construct
  source 1..12: note = 3F9[MICA/B]_H0L0 vhCDR3
DRRHYGSYAMDY

SEQ ID NO: 683 - protein, 112 aa, synthetic construct
  source 1..112: note = 3F9[MICA/B]_H0L0 Variable light (vl) domain
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 684 - protein, 16 aa, synthetic construct
  source 1..16: note = 3F9[MICA/B]_H0L0 vlCDR1
RSSKSLLHSNGNTYLY

SEQ ID NO: 685 - protein, 7 aa, synthetic construct
  source 1..7: note = 3F9[MICA/B]_H0L0 vlCDR2
RMSNLAS

SEQ ID NO: 686 - protein, 9 aa, synthetic construct
  source 1..9: note = 3F9[MICA/B]_H0L0 vlCDR3
MQHLEYPFT

SEQ ID NO: 687 - protein, 122 aa, synthetic construct
  source 1..122: note = 6E1[MICA/B]_H0L0 Variable heavy (vh) domain
QGQMQQSGAELVKPGASVKLSCKTSGFTFSDNYISWLKQKPGQSLEWIAWIYAGTGGSSYNQKFRDKAQLTVDTSSRTAYMQLSSLTTEDSAIYYCARHDYYGTSGAWFAYWGRGTLVTVSA

SEQ ID NO: 688 - protein, 5 aa, synthetic construct
  source 1..5: note = 6E1[MICA/B]_H0L0 vhCDR1
DNYIS

SEQ ID NO: 689 - protein, 17 aa, synthetic construct
  source 1..17: note = 6E1[MICA/B]_H0L0 vhCDR2
WIYAGTGGSSYNQKFRD

SEQ ID NO: 690 - protein, 13 aa, synthetic construct
  source 1..13: note = 6E1[MICA/B]_H0L0 vhCDR3
HDYYGTSGAWFAY

SEQ ID NO: 691 - protein, 112 aa, synthetic construct
  source 1..112: note = 6E1[MICA/B]_H0L0 Variable light (vl) domain
DVLMTQTPLSLPVSLGDQASISCRSSQHIVHSNENTYLEWYLQKPGQSPKLLIYKVSNRFSGVPDRFSGSGSGTDFTLKISRVEAEDLGVYYCFQGSHVPWTFGGGTKLEIK

SEQ ID NO: 692 - protein, 16 aa, synthetic construct
  source 1..16: note = 6E1[MICA/B]_H0L0 vlCDR1
RSSQHIVHSNENTYLE

SEQ ID NO: 693 - protein, 7 aa, synthetic construct
  source 1..7: note = 6E1[MICA/B]_H0L0 vlCDR2
KVSNRFS

SEQ ID NO: 694 - protein, 9 aa, synthetic construct
  source 1..9: note = 6E1[MICA/B]_H0L0 vlCDR3
FQGSHVPWT

SEQ ID NO: 695 - protein, 118 aa, synthetic construct
  source 1..118: note = 7C6[MICA/B]_H0L0 Variable heavy (vh) domain
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSS

SEQ ID NO: 696 - protein, 5 aa, synthetic construct
  source 1..5: note = 7C6[MICA/B]_H0L0 vhCDR1
NYAMN

SEQ ID NO: 697 - protein, 17 aa, synthetic construct
  source 1..17: note = 7C6[MICA/B]_H0L0 vhCDR2
WINTHTGDPTYADDFKG

SEQ ID NO: 698 - protein, 9 aa, synthetic construct
  source 1..9: note = 7C6[MICA/B]_H0L0 vhCDR3
TYGNYAMDY

SEQ ID NO: 699 - protein, 107 aa, synthetic construct
  source 1..107: note = 7C6[MICA/B]_H0L0 Variable light (vl) domain
DIQMTQTTSSLSASLGDRVTISCSASQDISNYLNWYQQKPDGTVKLLIYDTSILHLGVPSRFSGSGSGTDYSLTISNLEPEDIATYYCQQYSKFPRTFGGGTTLEIK

SEQ ID NO: 700 - protein, 11 aa, synthetic construct
  source 1..11: note = 7C6[MICA/B]_H0L0 vlCDR1
SASQDISNYLN

SEQ ID NO: 701 - protein, 7 aa, synthetic construct
  source 1..7: note = 7C6[MICA/B]_H0L0 vlCDR2
DTSILHL

SEQ ID NO: 702 - protein, 9 aa, synthetic construct
  source 1..9: note = 7C6[MICA/B]_H0L0 vlCDR3
QQYSKFPRT

SEQ ID NO: 703 - protein, 117 aa, synthetic construct
  source 1..117: note = 13A9 [MICA/B]_H0L0 Variable heavy (vh) domain
QVQLQQSGAELVRPGTSVKVSCKASGYAFTNYLIEWVKQRPGQGLEWIGAINPGSGATNYNEKFKDKARLTADKSSNTAYLQFSSLTSDDSAVYFCARFLGNYFDNWGQGATLTVSS

SEQ ID NO: 704 - protein, 10 aa, synthetic construct
  source 1..10: note = 13A9 [MICA/B]_H0L0 vhCDR1
GYAFTNYLIE

SEQ ID NO: 705 - protein, 17 aa, synthetic construct
  source 1..17: note = 13A9 [MICA/B]_H0L0 vhCDR2
AINPGSGATNYNEKFKD

SEQ ID NO: 706 - protein, 8 aa, synthetic construct
  source 1..8: note = 13A9 [MICA/B]_H0L0 vhCDR3
FLGNYFDN

SEQ ID NO: 707 - protein, 107 aa, synthetic construct
  source 1..107: note = 13A9 [MICA/B]_H0L0 Variable light (vl) domain
DIQMTQSPASLSASVGETVTITCRASGNIHSYLAWYQQKQGKSPQLLVYYAETLADGVPSRFSGRGSGTQYSLKINSLQPEDFGSYFCQQFWTTPYTFGGGTKVEIK

SEQ ID NO: 708 - protein, 11 aa, synthetic construct
  source 1..11: note = 13A9 [MICA/B]_H0L0 vlCDR1
RASGNIHSYLA

SEQ ID NO: 709 - protein, 7 aa, synthetic construct
  source 1..7: note = 13A9 [MICA/B]_H0L0 vlCDR2
YAETLAD

SEQ ID NO: 710 - protein, 9 aa, synthetic construct
  source 1..9: note = 13A9 [MICA/B]_H0L0 vlCDR3
QQFWTTPYT

SEQ ID NO: 711 - protein, 117 aa, synthetic construct
  source 1..117: note = 1D5 [MICA/B]_H0L0 Variable heavy (vh) domain
EIQLQQSGPELVKPGASVKVSCKASGYAFTSQNIYWVKQSHGKSLEWIGYIEPYNVVPMYNPKFKGKATLTVDKSSSSAYIHLNSLTSEDSAIYYCARSGSSNFDYWGQGTTLTVSS

SEQ ID NO: 712 - protein, 10 aa, synthetic construct
  source 1..10: note = 1D5 [MICA/B]_H0L0 vhCDR1
GYAFTSQNIY

SEQ ID NO: 713 - protein, 17 aa, synthetic construct
  source 1..17: note = 1D5 [MICA/B]_H0L0 vhCDR2
YIEPYNVVPMYNPKFKG

SEQ ID NO: 714 - protein, 8 aa, synthetic construct
  source 1..8: note = 1D5 [MICA/B]_H0L0 vhCDR3
SGSSNFDY

SEQ ID NO: 715 - protein, 108 aa, synthetic construct
  source 1..108: note = 1D5 [MICA/B]_H0L0 Variable light (vl) domain
EIILTQSPTTMAASPGEKITITCSASSSISSHYLHWYQQKSGFSPKLLIYRTSNLASGVPARFSGSGSGTSYSLTIGTMEAEDVATYYCQQGSSLPLTFGAGTKVEIK

SEQ ID NO: 716 - protein, 12 aa, synthetic construct
  source 1..12: note = 1D5 [MICA/B]_H0L0 vlCDR1
SASSSISSHYLH

SEQ ID NO: 717 - protein, 7 aa, synthetic construct
  source 1..7: note = 1D5 [MICA/B]_H0L0 vlCDR2
RTSNLAS

SEQ ID NO: 718 - protein, 9 aa, synthetic construct
  source 1..9: note = 1D5 [MICA/B]_H0L0 vlCDR3
QQGSSLPLT

SEQ ID NO: 719 - protein, 120 aa, synthetic construct
  source 1..120: note = 2C10A3.372[NKp46]_H1L1 Variable heavy (vh) domain
QVQLVQSGAEVKKPGSSVKVSCKASGGTFSNYAIHWVRQAPGQGLEWMGGIIPIFGLANYAQKFQGRVTITADESTSTAYMELSSLRSEDTAVYYCARRGALWGNAFDYWGQGTLVTVSS

SEQ ID NO: 720 - protein, 10 aa, synthetic construct
  source 1..10: note = 2C10A3.372[NKp46]_H1L1 vhCDR1
GGTFSNYAIH

SEQ ID NO: 721 - protein, 17 aa, synthetic construct
  source 1..17: note = 2C10A3.372[NKp46]_H1L1 vhCDR2
GIIPIFGLANYAQKFQG

SEQ ID NO: 722 - protein, 11 aa, synthetic construct
  source 1..11: note = 2C10A3.372[NKp46]_H1L1 vhCDR3
RGALWGNAFDY

SEQ ID NO: 723 - protein, 107 aa, synthetic construct
  source 1..107: note = 2C10A3.372[NKp46]_H1L1 Variable light (vl) domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 724 - protein, 11 aa, synthetic construct
  source 1..11: note = 2C10A3.372[NKp46]_H1L1 vlCDR1
RASQSISSYLN

SEQ ID NO: 725 - protein, 7 aa, synthetic construct
  source 1..7: note = 2C10A3.372[NKp46]_H1L1 vlCDR2
AASSLQS

SEQ ID NO: 726 - protein, 9 aa, synthetic construct
  source 1..9: note = 2C10A3.372[NKp46]_H1L1 vlCDR3
QQSYSTPYT

SEQ ID NO: 727 - protein, 117 aa, synthetic construct
  source 1..117: note = NKp46-A[NKp46] Variable heavy (vh) domain
QVQLVQSGAEVKKPGASVKVSCKASGYTFTSFTMHWVRQAPGQGLEWIGYINPSSGYTEYNQKFKDRVTITADKSTSTAYMELSSLRSEDTAVYYCVRGSSRGFDYWGQGTLVTVSS

SEQ ID NO: 728 - protein, 10 aa, synthetic construct
  source 1..10: note = NKp46-A[NKp46] vhCDR1
GYTFTSFTMH

SEQ ID NO: 729 - protein, 17 aa, synthetic construct
  source 1..17: note = NKp46-A[NKp46] vhCDR2
YINPSSGYTEYNQKFKD

SEQ ID NO: 730 - protein, 8 aa, synthetic construct
  source 1..8: note = NKp46-A[NKp46] vhCDR3
GSSRGFDY

SEQ ID NO: 731 - protein, 107 aa, synthetic construct
  source 1..107: note = NKp46-A[NKp46] Variable light (vl) domain
DIQMTQSPSSLSASVGDRVTITCRASENIYSNLAWFQQKPGKAPKLLVYAATNLADGVPSRFSGSGSGTDYTLTISSLQPEDFATYYCQHFWGTPRTFGGGTKVEIK

SEQ ID NO: 732 - protein, 11 aa, synthetic construct
  source 1..11: note = NKp46-A[NKp46] vlCDR1
RASENIYSNLA

SEQ ID NO: 733 - protein, 7 aa, synthetic construct
  source 1..7: note = NKp46-A[NKp46] vlCDR2
AATNLAD

SEQ ID NO: 734 - protein, 9 aa, synthetic construct
  source 1..9: note = NKp46-A[NKp46] vlCDR3
QHFWGTPRT

SEQ ID NO: 735 - protein, 679 aa, synthetic construct
  source 1..679: note = XENP47441 Chain 1 - 7C6[MICA/B]_H0_IgG1_pI(-)_Isosteric_A_L368D/K370S
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCGGGGSGGGGSQIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 736 - protein, 478 aa, synthetic construct
  source 1..478: note = XENP47441 Chain 2 - 7C6[MICA/B]_H0_Numax[RSV]_H1L1_ scFv(GKPGS)4_Fc(216)_IgG1_C220S/S364K/E357Q
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSGKPGSGKPGSGKPGSGKPGSDIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 737 - protein, 214 aa, synthetic construct
  source 1..214: note = XENP47441 Chain 3 - 7C6[MICA/B]_L0 Light Chain
DIQMTQTTSSLSASLGDRVTISCSASQDISNYLNWYQQKPDGTVKLLIYDTSILHLGVPSRFSGSGSGTDYSLTISNLEPEDIATYYCQQYSKFPRTFGGGTTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 738 - protein, 448 aa, synthetic construct
  source 1..448: note = XENP40894_7C6[MICA/B]_H0_L0_IgG1 Heavy Chain
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 739 - protein, 214 aa, synthetic construct; identical to SEQ ID NO: 737
  source 1..214: note = XENP40894_7C6[MICA/B]_H0_L0_IgG1 Light Chain

SEQ ID NO: 740 - protein, 447 aa, synthetic construct
  source 1..447: note = XENP47444 Chain 1 - 7C6[MICAB]_H0_IgG1_pI(-)_Isoste ric_A_PVA_/S267K/L368D/K370S
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 741 - protein, 707 aa, synthetic construct
  source 1..707: note = XENP47444 Chain 2 - 7C6[MICAB]_H0_IgG1_PVA_/S267K/S 364K/E357Q/K447del_(G4S)3_Numax[RSV]_H1L1_scFv(GKPGS)4
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSGKPGSGKPGSGKPGSGKPGSDIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIK

SEQ ID NO: 742 - protein, 214 aa, synthetic construct; identical to SEQ ID NO: 737
  source 1..214: note = XENP47444 Chain 3 - 7C6[MICAB]_L0

SEQ ID NO: 743 - protein, 448 aa, synthetic construct
  source 1..448: note = XENP47445 Chain 1 - 7C6[MICAB]_H0_IgG1_pI(-)_Isosteric_A_L368D/K370S
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 744 - protein, 708 aa, synthetic construct
  source 1..708: note = XENP47445 Chain 2 - 7C6[MICAB]_H0_IgG1_S364K/E357Q/ K447del_(G4S)3_Numax[RSV]_H1L1_scFv(GKPGS)4
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSGKPGSGKPGSGKPGSGKPGSDIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIK

SEQ ID NO: 745 - protein, 214 aa, synthetic construct; identical to SEQ ID NO: 737
  source 1..214: note = XENP47445 Chain 3 - 7C6[MICAB]_L0

SEQ ID NO: 746 - protein, 447 aa, synthetic construct
  source 1..447: note = XENP41021 Chain 1 - 38E2[B7H3]_H2_IgG1_pI(-)_Isoste ric_A_L368D/K370S-Fc(216)_IgG1_C220S/S364K/E357Q
EVQLLESGGGLVQPGGSLRLSCAVSGFSLSSYGVIWVRQAPGKGLEYVSWVSSGGSTNYASSVKGRFTISRDTSKNTVYLQMNSLRAEDTAVYFCARGATNSGTDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 747 - protein, 479 aa, synthetic construct
  source 1..479: note = XENP41021 Chain 2 - 38E2[B7H3]_H2_L1.1_scFv(GKPGS)4_IgG1_C220S/S364K/E357Q
EVQLLESGGGLVQPGGSLRLSCAVSGFSLSSYGVIWVRQAPGKGLEYVSWVSSGGSTNYASSVKGRFTISRDTSKNTVYLQMNSLRAEDTAVYFCARGATNSGTDIWGPGTLVTVSSGKPGSGKPGSGKPGSGKPGSDIVMTQSPSSLSASVGDRVTITCRASQSINSWLSWYQQKPGKPPKLLIYGASNLASGVPSRFKGSGSGTDFTLTISSLQPEDFATYYCQNNYGIIAAGATFGGGTKVEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 748 - protein, 217 aa, synthetic construct
  source 1..217: note = XENP41021 Chain 3 - 38E2[B7H3]_L1.1_Light Chain
DIVMTQSPSSLSASVGDRVTITCRASQSINSWLSWYQQKPGKPPKLLIYGASNLASGVPSRFKGSGSGTDFTLTISSLQPEDFATYYCQNNYGIIAAGATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 749 - protein, 447 aa, synthetic construct
  source 1..447: note = XENP41022 Chain 1 - 38E2[B7H3]_H2_IgG1_pI(-)_Isoste ric_A_S239D/I332E/L368D/K370S
EVQLLESGGGLVQPGGSLRLSCAVSGFSLSSYGVIWVRQAPGKGLEYVSWVSSGGSTNYASSVKGRFTISRDTSKNTVYLQMNSLRAEDTAVYFCARGATNSGTDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 750 - protein, 479 aa, synthetic construct
  source 1..479: note = XENP41022 Chain 2 - 38E2[B7H3]_H2_L1.1_scFv(GKPGS)4 -Fc(216)_IgG1_C220S/S239D/I332E/S364K/E357Q
EVQLLESGGGLVQPGGSLRLSCAVSGFSLSSYGVIWVRQAPGKGLEYVSWVSSGGSTNYASSVKGRFTISRDTSKNTVYLQMNSLRAEDTAVYFCARGATNSGTDIWGPGTLVTVSSGKPGSGKPGSGKPGSGKPGSDIVMTQSPSSLSASVGDRVTITCRASQSINSWLSWYQQKPGKPPKLLIYGASNLASGVPSRFKGSGSGTDFTLTISSLQPEDFATYYCQNNYGIIAAGATFGGGTKVEIKEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 751 - protein, 217 aa, synthetic construct; identical to SEQ ID NO: 748
  source 1..217: note = XENP41022 Chain 3 - 38E2[B7H3]_L1.1_Light Chain

SEQ ID NO: 752 - protein, 447 aa, synthetic construct
  source 1..447: note = XENP41023 Chain 1 - 38E2[B7H3]_H2_IgG1_pI(-)_Isosteric_A_L368D/K370S/S239D
EVQLLESGGGLVQPGGSLRLSCAVSGFSLSSYGVIWVRQAPGKGLEYVSWVSSGGSTNYASSVKGRFTISRDTSKNTVYLQMNSLRAEDTAVYFCARGATNSGTDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 753 - protein, 479 aa, synthetic construct
  source 1..479: note = XENP41023 Chain 2 - 38E2[B7H3]_H2_L1.1_scFv(GKPGS)4 -Fc(216)_IgG1_C220S/S364K/E357Q/S239D
EVQLLESGGGLVQPGGSLRLSCAVSGFSLSSYGVIWVRQAPGKGLEYVSWVSSGGSTNYASSVKGRFTISRDTSKNTVYLQMNSLRAEDTAVYFCARGATNSGTDIWGPGTLVTVSSGKPGSGKPGSGKPGSGKPGSDIVMTQSPSSLSASVGDRVTITCRASQSINSWLSWYQQKPGKPPKLLIYGASNLASGVPSRFKGSGSGTDFTLTISSLQPEDFATYYCQNNYGIIAAGATFGGGTKVEIKEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 754 - protein, 217 aa, synthetic construct; identical to SEQ ID NO: 748
  source 1..217: note = XENP41023 Chain 3 - 38E2[B7H3]_L1.1_Light Chain

SEQ ID NO: 755 - protein, 447 aa, synthetic construct
  source 1..447: note = XENP41024 Chain 1 - 38E2[B7H3]_H2_IgG1_pI(-)_Isosteric_A_L368D/K370S/I332E
EVQLLESGGGLVQPGGSLRLSCAVSGFSLSSYGVIWVRQAPGKGLEYVSWVSSGGSTNYASSVKGRFTISRDTSKNTVYLQMNSLRAEDTAVYFCARGATNSGTDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 756 - protein, 479 aa, synthetic construct
  source 1..479: note = XENP41024 Chain 2 - 38E2[B7H3]_H2_L1.1_scFv(GKPGS)4 -Fc(216)_IgG1_C220S/S364K/E357Q/I332E
EVQLLESGGGLVQPGGSLRLSCAVSGFSLSSYGVIWVRQAPGKGLEYVSWVSSGGSTNYASSVKGRFTISRDTSKNTVYLQMNSLRAEDTAVYFCARGATNSGTDIWGPGTLVTVSSGKPGSGKPGSGKPGSGKPGSDIVMTQSPSSLSASVGDRVTITCRASQSINSWLSWYQQKPGKPPKLLIYGASNLASGVPSRFKGSGSGTDFTLTISSLQPEDFATYYCQNNYGIIAAGATFGGGTKVEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 757 - protein, 217 aa, synthetic construct; identical to SEQ ID NO: 748
  source 1..217: note = XENP41024 Chain 3 - 38E2[B7H3]_L1.1_Light Chain

SEQ ID NO: 758 - protein, 447 aa, synthetic construct; identical to SEQ ID NO: 746
  source 1..447: note = XENP41025 Chain 1 - 38E2[B7H3]_H2_IgG1_pI(-)_Isosteric_A_L368D/K370S

SEQ ID NO: 759 - protein, 479 aa, synthetic construct; identical to SEQ ID NO: 750
  source 1..479: note = XENP41025 Chain 2 - 38E2[B7H3]_H2_L1.1_scFv(GKPGS)4 -Fc(216)_IgG1_C220S/S239D/I332E/S364K/E357Q

SEQ ID NO: 760 - protein, 217 aa, synthetic construct; identical to SEQ ID NO: 748
  source 1..217: note = XENP41025 Chain 3 - 38E2[B7H3]_L1.1_Light Chain

SEQ ID NO: 761 - protein, 447 aa, synthetic construct; identical to SEQ ID NO: 746
  source 1..447: note = XENP41026 Chain 1 - 38E2[B7H3]_H2_IgG1_pI(-)_Isosteric_A_L368D/K370S

SEQ ID NO: 762 - protein, 479 aa, synthetic construct; identical to SEQ ID NO: 753
  source 1..479: note = XENP41026 Chain 2 - 38E2[B7H3]_H2_L1.1_scFv(GKPGS)4 -Fc(216)_IgG1_C220S/S364K/E357Q/S239D

SEQ ID NO: 763 - protein, 217 aa, synthetic construct; identical to SEQ ID NO: 748
  source 1..217: note = XENP41026 Chain 3 - 38E2[B7H3]_L1.1_Light Chain

SEQ ID NO: 764 - protein, 447 aa, synthetic construct; identical to SEQ ID NO: 746
  source 1..447: note = XENP41027 Chain 1 - 38E2[B7H3]_H2_IgG1_pI(-)_Isosteric_A_L368D/K370S

SEQ ID NO: 765 - protein, 479 aa, synthetic construct; identical to SEQ ID NO: 756
  source 1..479: note = XENP41027 Chain 2 - 38E2[B7H3]_H2_L1.1_scFv(GKPGS)4 -Fc(216)_IgG1_C220S/S364K/E357Q/I332E

SEQ ID NO: 766 - protein, 217 aa, synthetic construct; identical to SEQ ID NO: 748
  source 1..217: note = XENP41027 Chain 3 - 38E2[B7H3]_L1.1_Light Chain

SEQ ID NO: 767 - protein, 447 aa, synthetic construct; identical to SEQ ID NO: 749
  source 1..447: note = XENP41028 Chain 1 - 38E2[B7H3]_H2_IgG1_pI(-)_Isoste ric_A_S239D/I332E/L368D/K370S

SEQ ID NO: 768 - protein, 479 aa, synthetic construct; identical to SEQ ID NO: 747
  source 1..479: note = XENP41028 Chain 2 - 38E2[B7H3]_H2_L1.1_scFv(GKPGS)4 -Fc(216)_IgG1_C220S/S364K/E357Q

SEQ ID NO: 769 - protein, 217 aa, synthetic construct; identical to SEQ ID NO: 748
  source 1..217: note = XENP41028 Chain 3 - 38E2[B7H3]_L1.1_Light Chain

SEQ ID NO: 770 - protein, 447 aa, synthetic construct; identical to SEQ ID NO: 752
  source 1..447: note = XENP41029 Chain 1 - 38E2[B7H3]_H2_IgG1_pI(-)_Isosteric_A_L368D/K370S/S239D

SEQ ID NO: 771 - protein, 479 aa, synthetic construct; identical to SEQ ID NO: 747
  source 1..479: note = XENP41029 Chain 2 - 38E2[B7H3]_H2_L1.1_scFv(GKPGS)4 -Fc(216)_IgG1_C220S/S364K/E357Q

SEQ ID NO: 772 - protein, 217 aa, synthetic construct; identical to SEQ ID NO: 748
  source 1..217: note = XENP41029 Chain 3 - 38E2[B7H3]_L1.1_Light Chain

SEQ ID NO: 773 - protein, 447 aa, synthetic construct; identical to SEQ ID NO: 755
  source 1..447: note = XENP41030 Chain 1 - 38E2[B7H3]_H2_IgG1_pI(-)_Isosteric_A_L368D/K370S/I332E

SEQ ID NO: 774 - protein, 479 aa, synthetic construct; identical to SEQ ID NO: 747
  source 1..479: note = XENP41030 Chain 2 - 38E2[B7H3]_H2_L1.1_scFv(GKPGS)4 -Fc(216)_IgG1_C220S/S364K/E357Q

SEQ ID NO: 775 - protein, 217 aa, synthetic construct; identical to SEQ ID NO: 748
  source 1..217: note = XENP41030 Chain 3 - 38E2[B7H3]_L1.1_Light Chain

SEQ ID NO: 776 - protein, 447 aa, synthetic construct; identical to SEQ ID NO: 749
  source 1..447: note = XENP41031 Chain 1 - 38E2[B7H3]_H2_IgG1_pI(-)_Isoste ric_A_S239D/I332E/L368D/K370S

SEQ ID NO: 777 - protein, 479 aa, synthetic construct; identical to SEQ ID NO: 753
  source 1..479: note = XENP41031 Chain 2 - 38E2[B7H3]_H2_L1.1_scFv(GKPGS)4 -Fc(216)_IgG1_C220S/S364K/E357Q/S239D

SEQ ID NO: 778 - protein, 217 aa, synthetic construct; identical to SEQ ID NO: 748
  source 1..217: note = XENP41031 Chain 3 - 38E2[B7H3]_L1.1_Light Chain

SEQ ID NO: 779 - protein, 447 aa, synthetic construct; identical to SEQ ID NO: 749
  source 1..447: note = XENP41032 Chain 1 - 38E2[B7H3]_H2_IgG1_pI(-)_Isoste ric_A_S239D/I332E/L368D/K370S

SEQ ID NO: 780 - protein, 479 aa, synthetic construct; identical to SEQ ID NO: 756
  source 1..479: note = XENP41032 Chain 2 - 38E2[B7H3]_H2_L1.1_scFv(GKPGS)4 -Fc(216)_IgG1_C220S/S364K/E357Q/I332E

SEQ ID NO: 781 - protein, 217 aa, synthetic construct; identical to SEQ ID NO: 748
  source 1..217: note = XENP41032 Chain 3 - 38E2[B7H3]_L1.1_Light Chain

SEQ ID NO: 782 - protein, 447 aa, synthetic construct; identical to SEQ ID NO: 752
  source 1..447: note = XENP41033 Chain 1 - 38E2[B7H3]_H2_IgG1_pI(-)_Isosteric_A_L368D/K370S/S239D

SEQ ID NO: 783 - protein, 479 aa, synthetic construct; identical to SEQ ID NO: 750
  source 1..479: note = XENP41033 Chain 2 - 38E2[B7H3]_H2_L1.1_scFv(GKPGS)4 -Fc(216)_IgG1_C220S/S239D/I332E/S364K/E357Q

SEQ ID NO: 784 - protein, 217 aa, synthetic construct; identical to SEQ ID NO: 748
  source 1..217: note = XENP41033 Chain 3 - 38E2[B7H3]_L1.1_Light Chain

SEQ ID NO: 785 - protein, 447 aa, synthetic construct; identical to SEQ ID NO: 755
  source 1..447: note = XENP41034 Chain 1 - 38E2[B7H3]_H2_IgG1_pI(-)_Isosteric_A_L368D/K370S/I332E

SEQ ID NO: 786 - protein, 479 aa, synthetic construct; identical to SEQ ID NO: 750
  source 1..479: note = XENP41034 Chain 2 - 38E2[B7H3]_H2_L1.1_scFv(GKPGS)4 -Fc(216)_IgG1_C220S/S239D/I332E/S364K/E357Q

SEQ ID NO: 787 - protein, 217 aa, synthetic construct; identical to SEQ ID NO: 748
  source 1..217: note = XENP41034 Chain 3 - 38E2[B7H3]_L1.1_Light Chain

SEQ ID NO: 788 - protein, 447 aa, synthetic construct; identical to SEQ ID NO: 752
  source 1..447: note = XENP41035 Chain 1 - 38E2[B7H3]_H2_IgG1_pI(-)_Isosteric_A_L368D/K370S/S239D

SEQ ID NO: 789 - protein, 479 aa, synthetic construct; identical to SEQ ID NO: 756
  source 1..479: note = XENP41035 Chain 2 - 38E2[B7H3]_H2_L1.1_scFv(GKPGS)4 -Fc(216)_IgG1_C220S/S364K/E357Q/I332E

SEQ ID NO: 790 - protein, 217 aa, synthetic construct; identical to SEQ ID NO: 748
  source 1..217: note = XENP41035 Chain 3 - 38E2[B7H3]_L1.1_Light Chain

SEQ ID NO: 791 - protein, 447 aa, synthetic construct; identical to SEQ ID NO: 755
  source 1..447: note = XENP41036 Chain 1 - 38E2[B7H3]_H2_IgG1_pI(-)_Isosteric_A_L368D/K370S/I332E

SEQ ID NO: 792 - protein, 479 aa, synthetic construct; identical to SEQ ID NO: 753
  source 1..479: note = XENP41036 Chain 2 - 38E2[B7H3]_H2_L1.1_scFv(GKPGS)4 -Fc(216)_IgG1_C220S/S364K/E357Q/S239D

SEQ ID NO: 793 - protein, 217 aa, synthetic construct; identical to SEQ ID NO: 748
  source 1..217: note = XENP41036 Chain 3 - 38E2[B7H3]_L1.1_Light Chain

SEQ ID NO: 794 - protein, 447 aa, synthetic construct; identical to SEQ ID NO: 740
  source 1..447: note = XENP46810 Chain 1 - 7C6[MICA/B]_H0_IgG1_pI(-)_Isost eric_A_PVA_/S267K/L368D/K370S

SEQ ID NO: 795 - protein, 478 aa, synthetic construct
  source 1..478: note = XENP46810 Chain 2 - 2C10A3.372[NKp46]_H1L1_scFv(GKP GS)4_Fc(216)_IgG1_C220S/PVA_/S267K/S364K/E357Q
QVQLVQSGAEVKKPGSSVKVSCKASGGTFSNYAIHWVRQAPGQGLEWMGGIIPIFGLANYAQKFQGRVTITADESTSTAYMELSSLRSEDTAVYYCARRGALWGNAFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 796 - protein, 214 aa, synthetic construct; identical to SEQ ID NO: 737
  source 1..214: note = XENP46810 Chain 3 - 7C6[MICA/B]_L0 Light Chain

SEQ ID NO: 797 - protein, 448 aa, synthetic construct; identical to SEQ ID NO: 743
  source 1..448: note = XENP46811 Chain 1 - 7C6[MICA/B]_H0_IgG1_pI(-)_Isosteric_A_L368D/K370S

SEQ ID NO: 798 - protein, 479 aa, synthetic construct
  source 1..479: note = XENP46811 Chain 2 - 2C10A3.372[NKp46]_H1L1_scFv(GKP GS)4_Fc(216)_IgG1_C220S/S364K/E357Q
QVQLVQSGAEVKKPGSSVKVSCKASGGTFSNYAIHWVRQAPGQGLEWMGGIIPIFGLANYAQKFQGRVTITADESTSTAYMELSSLRSEDTAVYYCARRGALWGNAFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 799 - protein, 214 aa, synthetic construct; identical to SEQ ID NO: 737
  source 1..214: note = XENP46811 Chain 3 - 7C6[MICA/B]_L0 Light Chain

SEQ ID NO: 800 - protein, 448 aa, synthetic construct
  source 1..448: note = XENP46812 Chain 1 - 7C6[MICA/B]_H0_IgG1_pI(-)_Isost eric_A_S239D/I332E/L368D/K370S
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 801 - protein, 479 aa, synthetic construct
  source 1..479: note = XENP46812 Chain 2 - 2C10A3.372[NKp46]_H1L1_scFv(GKP GS)4_Fc(216)_IgG1_C220S/S239D/I332E/S364K/E357Q
QVQLVQSGAEVKKPGSSVKVSCKASGGTFSNYAIHWVRQAPGQGLEWMGGIIPIFGLANYAQKFQGRVTITADESTSTAYMELSSLRSEDTAVYYCARRGALWGNAFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 802 - protein, 214 aa, synthetic construct; identical to SEQ ID NO: 737
  source 1..214: note = XENP46812 Chain 3 - 7C6[MICA/B]_L0 Light Chain

SEQ ID NO: 803 - protein, 231 aa, synthetic construct
  source 1..231: note = XENP47274 Chain 1 - empty-Fc(216)_IgG1_pI(-)_Isoste ric_A_C220S/PVA_/S267K/L368D/K370S
EPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 804 - protein, 713 aa, synthetic construct
  source 1..713: note = XENP47274 Chain 2 - 2C10A3.372[NKp46]_H1_(G4S)2_7C6 [MICAB]_H0L0_scFv(GKPGS)4_(G4S)2_Fc(222)_IgG1_PVA_/S267K/S 364K/E357Q
QVQLVQSGAEVKKPGSSVKVSCKASGGTFSNYAIHWVRQAPGQGLEWMGGIIPIFGLANYAQKFQGRVTITADESTSTAYMELSSLRSEDTAVYYCARRGALWGNAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSGKPGSGKPGSGKPGSGKPGSDIQMTQTTSSLSASLGDRVTISCSASQDISNYLNWYQQKPDGTVKLLIYDTSILHLGVPSRFSGSGSGTDYSLTISNLEPEDIATYYCQQYSKFPRTFGGGTTLEIKGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 805 - protein, 214 aa, synthetic construct
  source 1..214: note = XENP47274 Chain 3 - 2C10A3.372[NKp46]_L1 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 806 - protein, 232 aa, synthetic construct
  source 1..232: note = XENP47281 Chain 1 - empty-Fc(216)_IgG1_pI(-)_Isosteric_A_C220S/L368D/K370S
EPKSSDKTHTCPPCPAPELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 807 - protein, 714 aa, synthetic construct
  source 1..714: note = XENP47281 Chain 2 - 2C10A3.372[NKp46]_H1_(G4S)2_7C6 [MICAB]_H0L0_scFv(GKPGS)4_(G4S)2_Fc(222)_IgG1_S364K/E357Q
QVQLVQSGAEVKKPGSSVKVSCKASGGTFSNYAIHWVRQAPGQGLEWMGGIIPIFGLANYAQKFQGRVTITADESTSTAYMELSSLRSEDTAVYYCARRGALWGNAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSGKPGSGKPGSGKPGSGKPGSDIQMTQTTSSLSASLGDRVTISCSASQDISNYLNWYQQKPDGTVKLLIYDTSILHLGVPSRFSGSGSGTDYSLTISNLEPEDIATYYCQQYSKFPRTFGGGTTLEIKGGGGSGGGGSKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 808 - protein, 214 aa, synthetic construct; identical to SEQ ID NO: 805
  source 1..214: note = XENP47281 Chain 3 - 2C10A3.372[NKp46]_L1 Light Chain

SEQ ID NO: 809 - protein, 447 aa, synthetic construct; identical to SEQ ID NO: 740
  source 1..447: note = XENP47438 Chain 1 - 7C6[MICAB]_H0_IgG1_pI(-)_Isoste ric_A_PVA_/S267K/L368D/K370S

SEQ ID NO: 810 - protein, 713 aa, synthetic construct
  source 1..713: note = XENP47438 Chain 2 - 7C6[MICAB]_H0_(G4S)2_2C10A3.372 [NKp46]_H1L1_scFv(GKPGS)4_(G4S)2_Fc(222)_IgG1_/PVA_/S267K/ S364K/E357Q
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLVQSGAEVKKPGSSVKVSCKASGGTFSNYAIHWVRQAPGQGLEWMGGIIPIFGLANYAQKFQGRVTITADESTSTAYMELSSLRSEDTAVYYCARRGALWGNAFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKGGGGSGGGGSKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 811 - protein, 214 aa, synthetic construct; identical to SEQ ID NO: 737
  source 1..214: note = XENP47438 Chain 3 - 7C6[MICA/B]_L0 Light Chain

SEQ ID NO: 812 - protein, 448 aa, synthetic construct; identical to SEQ ID NO: 743
  source 1..448: note = XENP47439 Chain 1 - 7C6[MICAB]_H0_IgG1_pI(-)_Isosteric_A_L368D/K370S

SEQ ID NO: 813 - protein, 714 aa, synthetic construct
  source 1..714: note = XENP47439 Chain 2 - 7C6[MICAB]_H0_(G4S)2_2C10A3.372 [NKp46]_H1L1_scFv(GKPGS)4_(G4S)2_Fc(222)_IgG1_S364K/E357Q
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLVQSGAEVKKPGSSVKVSCKASGGTFSNYAIHWVRQAPGQGLEWMGGIIPIFGLANYAQKFQGRVTITADESTSTAYMELSSLRSEDTAVYYCARRGALWGNAFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKGGGGSGGGGSKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 814 - protein, 214 aa, synthetic construct; identical to SEQ ID NO: 737
  source 1..214: note = XENP47439 Chain 3 - 7C6[MICA/B]_L0 Light Chain

SEQ ID NO: 815 - protein, 678 aa, synthetic construct
  source 1..678: note = XENP47442 Chain 1 - 7C6[MICA/B]_H0_(G4S)2_7C6[MICA/ B]_H0_IgG1_pI(-)_Isosteric_A_PVA_/S267K/L368D/K370S
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCGGGGSGGGGSQIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 816 - protein, 478 aa, synthetic construct; identical to SEQ ID NO: 795
  source 1..478: note = XENP47442 Chain 2 - 2C10A3.372[NKp46]_H1L1_scFv(GKP GS)4_Fc(216)_IgG1_C220S/PVA_/S267K/S364K/E357Q

SEQ ID NO: 817 - protein, 214 aa, synthetic construct; identical to SEQ ID NO: 737
  source 1..214: note = XENP47442 Chain 3 - 7C6[MICA/B]_L0 Light Chain

SEQ ID NO: 818 - protein, 679 aa, synthetic construct; identical to SEQ ID NO: 735
  source 1..679: note = XENP47443 Chain 1 - 7C6[MICA/B]_H0_(G4S)2_7C6[MICA/ B]_H0_IgG1_pI(-)_Isosteric_A_L368D/K370S

SEQ ID NO: 819 - protein, 479 aa, synthetic construct; identical to SEQ ID NO: 798
  source 1..479: note = XENP47443 Chain 2 - 2C10A3.372[NKp46]_H1L1_scFv(GKP GS)4_Fc(216)_IgG1_C220S/S364K/E357Q

SEQ ID NO: 820 - protein, 214 aa, synthetic construct; identical to SEQ ID NO: 737
  source 1..214: note = XENP47443 Chain 3 - 7C6[MICA/B]_L0 Light Chain

SEQ ID NO: 821 - protein, 447 aa, synthetic construct; identical to SEQ ID NO: 740
  source 1..447: note = XENP47446 Chain 1 - 7C6[MICAB]_H0_IgG1_pI(-)_Isoste ric_A_PVA_/S267K/L368D/K370S

SEQ ID NO: 822 - protein, 708 aa, synthetic construct
  source 1..708: note = XENP47446 Chain 2 - 7C6[MICAB]_H0_IgG1_PVA_/S267K/S 364K/E357Q/K447del_(G4S)3_2C10A3.372[NKp46]_H1L1_scFv(GKPG S)4
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLVQSGAEVKKPGSSVKVSCKASGGTFSNYAIHWVRQAPGQGLEWMGGIIPIFGLANYAQKFQGRVTITADESTSTAYMELSSLRSEDTAVYYCARRGALWGNAFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 823 - protein, 214 aa, synthetic construct; identical to SEQ ID NO: 737
  source 1..214: note = XENP47446 Chain 3 - 7C6[MICA/B]_L0 Light Chain

SEQ ID NO: 824 - protein, 448 aa, synthetic construct; identical to SEQ ID NO: 743
  source 1..448: note = XENP47447 Chain 1 - 7C6[MICAB]_H0_IgG1_pI(-)_Isosteric_A_L368D/K370S

SEQ ID NO: 825 - protein, 709 aa, synthetic construct
  source 1..709: note = XENP47447 Chain 2 - 7C6[MICAB]_H0_IgG1_S364K/E357Q/ K447del_(G4S)3_2C10A3.372[NKp46]_H1L1_scFv(GKPGS)4
QIQLVQSGPELKKPGETVKVSCKASGYMFTNYAMNWVKQAPEKGLKWMGWINTHTGDPTYADDFKGRIAFSLETSASTAYLQINNLKNEDTATYFCVRTYGNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLVQSGAEVKKPGSSVKVSCKASGGTFSNYAIHWVRQAPGQGLEWMGGIIPIFGLANYAQKFQGRVTITADESTSTAYMELSSLRSEDTAVYYCARRGALWGNAFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 826 - protein, 214 aa, synthetic construct; identical to SEQ ID NO: 737
  source 1..214: note = XENP47447 Chain 3 - 7C6[MICA/B]_L0 Light Chain

SEQ ID NO: 827 - protein, 93 aa, synthetic construct
  source 1..93: note = Consensus Identity
VPPMVNVTRSEASEGNITVTCRASSFYPRNITLTWRQDGVSLSHDTQQWGDVLPDGNGTYQTWVATRICQGEEQRFTCYMEHSGNHSTHPVPS

SEQ ID NO: 828 - protein, 93 aa, Homo sapiens
  source 1..93: note = MICB*004 a3
VPPMVNVICSEVSEGNITVTCRASSFYPRNITLTWRQDGVSLSHNTQQWGDVLPDGNGTYQTWVATRIRQGEEQRFTCYMEHSGNHGTHPVPS

SEQ ID NO: 829 - protein, 93 aa, Homo sapiens
  source 1..93: note = MICB*005 a3
VPPMVNVTCSEVSEGNITVTCRASSFYPRNITLTWRQDGVSLSHNTQQWGDVLPDGNGTYQTWVATRIRQGEEQRFTCYMEHSGNHGTHPVPS

SEQ ID NO: 830 - protein, 93 aa, Homo sapiens
  source 1..93: note = MICA*002 a3
VPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 831 - protein, 93 aa, Homo sapiens; identical to SEQ ID NO: 827
  source 1..93: note = MICA*004 a3

SEQ ID NO: 832 - protein, 93 aa, Homo sapiens
  source 1..93: note = MICA*008 a3
VPPMVNVTRSEASEGNITVTCRASSFYPRNIILTWRQDGVSLSHDTQQWGDVLPDGNGTYQTWVATRICRGEEQRFTCYMEHSGNHSTHPVPS

SEQ ID NO: 833 - protein, 350 aa, synthetic construct
  source 1..350: note = XENP24045 Chain 1 - human_IL15_D30N/E64Q/N65D_(GGGG S)1_Fc(216)_IgG1_pI(-)_Isosteric_A_C220S/PVA_/S267K/L368D/ K370S
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 834 - protein, 301 aa, synthetic construct
  source 1..301: note = XENP24045 Chain 2 - human_IL15Ra(Sushi)_(GGGGS)1_Fc (216)_IgG1_C220S/PVA_/S267K/S364K/E357Q
ITCPPPMSVEHADIWVKSYSLYSRERYICNSGFKRKAGTSSLTECVLNKATNVAHWTTPSLKCIRGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 835 - protein, 450 aa, synthetic construct
  source 1..450: note = XENP43729 Chain 1
EVQLLESGGGLVQPGGSLRLSCAASGFTFSNYYMAWVRQAPGKGLEWVSSISTGGGSTYYRDSVKGRFTISRDNSKSTLYLQMNSLRAEDTAVYYCTTDLSAIAATPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 836 - protein, 477 aa, synthetic construct
  source 1..477: note = XENP43729 Chain 2
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSGKPGSGKPGSGKPGSGKPGSDIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 837 - protein, 213 aa, synthetic construct
  source 1..213: note = XENP43729 Chain 3
DIQMTQSPSSLSASVGDRVTITCRASQNIYTSLAWYQQKPGKAPKLLIYNAHSLQTGIPSRFSGSGSGADFTLTISSLQPEDFATYFCQQYYSGLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 838 - protein, 451 aa, synthetic construct
  source 1..451: note = XENP45899 Chain 1
EVQLLESGGGLVQPGGSLRLSCAASGFTFSNYYMAWVRQAPGKGLEWVSSISTGGGSTYYRDSVKGRFTISRDNSKSTLYLQMNSLRAEDTAVYYCTTDLSAIAATPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPELLGGPDVFLFPPKPKDTLMISRTPEVTCVVVDVSHEDPEVKFNWYVDGVEVHNAKTKPREEEYNSTYRVVSVLTVLHQDWLNGKEYKCKVSNKALPAPEEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 839 - protein, 479 aa, synthetic construct
  source 1..479: note = XENP45899 Chain 2
QVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSGKPGSGKPGSGKPGSGKPGSDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 840 - protein, 213 aa, synthetic construct; identical to SEQ ID NO: 837
  source 1..213: note = XENP45899 Chain 3

SEQ ID NO: 841 - protein, 451 aa, synthetic construct; identical to SEQ ID NO: 838
  source 1..451: note = XENP45900 Chain 1

SEQ ID NO: 842 - protein, 476 aa, synthetic construct
  source 1..476: note = XENP45900 Chain 2
QVQLVQSGAEVKKPGASVKVSCKASGYTFTSFTMHWVRQAPGQGLEWIGYINPSSGYTEYNQKFKDRVTITADKSTSTAYMELSSLRSEDTAVYYCVRGSSRGFDYWGQGTLVTVSSGKPGSGKPGSGKPGSGKPGSDIQMTQSPSSLSASVGDRVTITCRASENIYSNLAWFQQKPGKAPKLLVYAATNLADGVPSRFSGSGSGTDYTLTISSLQPEDFATYYCQHFWGTPRTFGGGTKVEIKEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 843 - protein, 213 aa, synthetic construct; identical to SEQ ID NO: 837
  source 1..213: note = XENP45900 Chain 3

SEQ ID NO: 844 - protein, 451 aa, synthetic construct; identical to SEQ ID NO: 838
  source 1..451: note = XENP43733 Chain 1

SEQ ID NO: 845 - protein, 478 aa, synthetic construct
  source 1..478: note = XENP43733 Chain 2
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSGKPGSGKPGSGKPGSGKPGSDIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 846 - protein, 213 aa, synthetic construct; identical to SEQ ID NO: 837
  source 1..213: note = XENP43733 Chain 3

SEQ ID NO: 847 - protein, 451 aa, synthetic construct
  source 1..451: note = XENP43730 Chain 1
EVQLLESGGGLVQPGGSLRLSCAASGFTFSNYYMAWVRQAPGKGLEWVSSISTGGGSTYYRDSVKGRFTISRDNSKSTLYLQMNSLRAEDTAVYYCTTDLSAIAATPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 848 - protein, 478 aa, synthetic construct; identical to SEQ ID NO: 736
  source 1..478: note = XENP43730 Chain 2

SEQ ID NO: 849 - protein, 213 aa, synthetic construct; identical to SEQ ID NO: 837
  source 1..213: note = XENP43730 Chain 3

SEQ ID NO: 850 - protein, 451 aa, synthetic construct; identical to SEQ ID NO: 838
  source 1..451: note = XENP45601 Chain 1

SEQ ID NO: 851 - protein, 474 aa, synthetic construct
  source 1..474: note = XENP45601 Chain 2
QVQLVQSGAEVKKPGASVKVSCKASGYTFTSYWMHWIRQAPGQGLEWMGHINPSTDYTAYNQKFQGRVTMTADKSTSTAYMELSSLRSEDTAVYYCALTGRFAYWGQGTLVTVSSGKPGSGKPGSGKPGSGKPGSDIQMTQSPSSLSASVGDRVTITCRASQDIGSSLNWLQQKPGKTIKRLIYATSSLDSGVPKRFSGSRSGSDYTLTISSLQPEDFATYYCLQYATSPFTFGSGTKLEIKEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 852 - protein, 213 aa, synthetic construct; identical to SEQ ID NO: 837
  source 1..213: note = XENP45601 Chain 3

SEQ ID NO: 853 - protein, 451 aa, synthetic construct; identical to SEQ ID NO: 838
  source 1..451: note = XENP43933 Chain 1

SEQ ID NO: 854 - protein, 481 aa, synthetic construct
  source 1..481: note = XENP43933 Chain 2
QVQLVQSGAEVKKPGASVKVSCKASGYTFTNYWMHWVRQAPGQGLEWMGEIDPSDIYTNYAQKFQGRVTMTRDTSTSTVYMELSSLRSEDTAVYYCARGIYDGYYVYGMDYWGQGTTVTVSSGKPGSGKPGSGKPGSGKPGSYIQMTQSPSSLSASVGDRVTITCRSSQDISNYLNWYQQKPGKAPKLLIYYTSRLHSGVPSRFSGSGSGTDFTFTISSLQPEDIATYYCQQGKTLPRTFGGGTKVEIKEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 855 - protein, 213 aa, synthetic construct; identical to SEQ ID NO: 837
  source 1..213: note = XENP43933 Chain 3

SEQ ID NO: 856 - protein, 451 aa, synthetic construct; identical to SEQ ID NO: 847
  source 1..451: note = XENP43995 Chain 1

SEQ ID NO: 857 - protein, 479 aa, synthetic construct
  source 1..479: note = XENP43995 Chain 2
QVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSGKPGSGKPGSGKPGSGKPGSDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 858 - protein, 213 aa, synthetic construct; identical to SEQ ID NO: 837
  source 1..213: note = XENP43995 Chain 3

SEQ ID NO: 859 - protein, 450 aa, synthetic construct; identical to SEQ ID NO: 835
  source 1..450: note = XENP44543 Chain 1

SEQ ID NO: 860 - protein, 478 aa, synthetic construct; identical to SEQ ID NO: 795
  source 1..478: note = XENP44543 Chain 2

SEQ ID NO: 861 - protein, 213 aa, synthetic construct; identical to SEQ ID NO: 837
  source 1..213: note = XENP44543 Chain 3

SEQ ID NO: 862 - protein, 451 aa, synthetic construct; identical to SEQ ID NO: 838
  source 1..451: note = XENP45903 Chain 1

SEQ ID NO: 863 - protein, 479 aa, synthetic construct; identical to SEQ ID NO: 801
  source 1..479: note = XENP45903 Chain 2

SEQ ID NO: 864 - protein, 213 aa, synthetic construct; identical to SEQ ID NO: 837
  source 1..213: note = XENP45903 Chain 3

SEQ ID NO: 865 - protein, 451 aa, synthetic construct; identical to SEQ ID NO: 838
  source 1..451: note = XENP45902 Chain 1

SEQ ID NO: 866 - protein, 489 aa, synthetic construct
  source 1..489: note = XENP45902 Chain 2
EVQLLESGGGLVQPGGSLRLSCAASGFTFSSYAMSWVRQAPGKGLEWVSAIGGTGGNTNYADSVKGRFTISRDNSKNTLYLQMNSLRAEDTAVYYCAKGAYSGSYYGLGLFDYWGQGTLVTVSSGKPGSGKPGSGKPGSGKPGSDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKLEIKEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 867 - protein, 213 aa, synthetic construct; identical to SEQ ID NO: 837
  source 1..213: note = XENP45902 Chain 3

SEQ ID NO: 868 - protein, 451 aa, synthetic construct; identical to SEQ ID NO: 847
  source 1..451: note = XENP44549 Chain 1

SEQ ID NO: 869 - protein, 479 aa, synthetic construct; identical to SEQ ID NO: 798
  source 1..479: note = XENP44549 Chain 2

SEQ ID NO: 870 - protein, 213 aa, synthetic construct; identical to SEQ ID NO: 837
  source 1..213: note = XENP44549 Chain 3

SEQ ID NO: 871 - protein, 451 aa, synthetic construct; identical to SEQ ID NO: 847
  source 1..451: note = XENP44551 Chain 1

SEQ ID NO: 872 - protein, 478 aa, synthetic construct
  source 1..478: note = XENP44551 Chain 2
QVQLVQSGAEVKKPGSSVKVSCKASGGTFSSYAISWVRQAPGQGLEWMGGIIPVFGSAHYAQKFQGRVTITADESTSTAYMELSSLRSEDTAVYYCAREDVARGYLDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 873 - protein, 213 aa, synthetic construct; identical to SEQ ID NO: 837
  source 1..213: note = XENP44551 Chain 3

SEQ ID NO: 874 - protein, 451 aa, synthetic construct; identical to SEQ ID NO: 847
  source 1..451: note = XENP44553 Chain 1

SEQ ID NO: 875 - protein, 489 aa, synthetic construct
  source 1..489: note = XENP44553 Chain 2
EVQLLESGGGLVQPGGSLRLSCAASGFTFSSYAMSWVRQAPGKGLEWVSAIGGTGGNTNYADSVKGRFTISRDNSKNTLYLQMNSLRAEDTAVYYCAKGAYSGSYYGLGLFDYWGQGTLVTVSSGKPGSGKPGSGKPGSGKPGSDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 876 - protein, 213 aa, synthetic construct; identical to SEQ ID NO: 837
  source 1..213: note = XENP44553 Chain 3

SEQ ID NO: 877 - protein, 451 aa, synthetic construct; identical to SEQ ID NO: 847
  source 1..451: note = XENP44552 Chain 1

SEQ ID NO: 878 - protein, 478 aa, synthetic construct
  source 1..478: note = XENP44552 Chain 2
EVQLLESGGGLVQPGGSLRLSCAASGFTFSGYAMSWVRQAPGKGLEWVSIISGSGATYYYADSVKGRFTISRDNSKNTLYLQMNSLRAEDTAVYYCAKEGYLYSQMDIWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 879 - protein, 213 aa, synthetic construct; identical to SEQ ID NO: 837
  source 1..213: note = XENP44552 Chain 3

(19) United States
(12) Patent Application Publication
(10) Pub. No.: US 2025/0243279 A1
(43) Pub. Date: Jul. 31, 2025

(54) BISPECIFIC ANTIBODIES THAT BIND TO NKP46 AND MICA/B

(71) Applicant: Xencor, Inc., Pasadena, CA (US)

(72) Inventors: Matthew S. FABER, Glendora, CA (US); Katrina BYKOVA, Seattle, WA (US); Tian ZHANG, Chino Hills, CA (US); Kendra AVERY, Hawthorne, CA (US); James WIELER, La Canada, CA (US); Jing QI, Arcadia, CA (US); Juan DIAZ, Anaheim Hills, CA (US); Elizabeth A. HENDERSON, Fullerton, CA (US); Su-shin HAO, Pasadena, CA (US)

(21) Appl. No.: 18/919,124

(22) Filed: Oct. 17, 2024

Related U.S. Application Data
(60) Provisional application No. 63/590,925, filed on Oct. 17, 2023.
(60) Provisional application No. 63/594,672, filed on Oct. 31, 2023.
(60) Provisional application No. 63/610,815, filed on Dec. 15, 2023.

(51) Int. Cl. C07K 16/28

(52) U.S. Cl. CPC C07K 16/2833; C07K 2317/31; C07K 2317/522; C07K 2317/524; C07K 2317/526; C07K 2317/53; C07K 2317/565; C07K 2317/622

(57) ABSTRACT

Provided herein are anti-NKp46×anti-MICA/B antibodies and methods of using such antibodies for the treatment of MICA/B-associated cancers, as well as anti-NKp46×anti-B7H3 antibodies.

### CROSS-REFERENCE

This application claims priority to U.S. Provisional Application No. 63/590,925, filed on Oct. 17, 2023, U.S. Provisional Application No. 63/594,672, filed on Oct. 31, 2023, and U.S. Provisional Application No. 63/610,815, filed on Dec. 15, 2023, which are incorporated herein by reference in their entirety.

### SEQUENCE LISTING

The instant application contains a Sequence Listing which has been submitted electronically in XML format and is hereby incorporated by reference in its entirety. Said XML copy, created on Dec. 6, 2024, is named 51096_4017_US_SL.xml and is 1,046,403 bytes in size.

### BACKGROUND

Antibody-based therapeutics have been used successfully to treat a variety of diseases, including cancer. An increasingly prevalent avenue being explored is the engineering of single immunoglobulin molecules that co-engage two different antigens. Such alternate antibody formats that engage two different antigens are often referred to as bispecific antibodies. Because the considerable diversity of the antibody variable region (Fv) makes it possible to produce an Fv that recognizes virtually any molecule, the typical approach to bispecific antibody generation is the introduction of new variable regions into the antibody.

Natural killer cell engagers (NKEs), a new class of immune-oncology therapeutics, contain fragments of antibodies such as antibody binding domains and are designed to exploit the immune functions of NK cells in cancer.

NK cells are part of the innate immune system and represent 5-20% of circulating lymphocytes in humans. NK cells infiltrate virtually all tissues and were originally characterized by their ability to kill tumor cells effectively without the need for prior sensitization. This ability to infiltrate also occurs in the tumor microenvironment and this infiltration is associated with better overall survival in patients.

Activated NK cells kill target cells by means similar to cytotoxic T cells, using cytolytic granules as well as through death receptor pathways. When NK cells encounter foreign or cancer cells, they are activated via there activating receptors, including NKp46. Thus, similar to “T Cell Engagers” (TCEs), that bind to CD3 on T Cells and a tumor antigen on a tumor cell to drive cytotoxic killing of the tumor cell, Natural Killer Engagers (NKEs) can be made that bind to the NK cells and a tumor cell and similarly facilitate cytotoxic killing.

Natural killer group 2 member D (NKG2D) is an activating receptor present on the surface of natural killer (NK) cells, some NK T cells, CD8+ cytotoxic T cells, 76 T cells, and CD4+ T cells, under certain conditions. (Champsaur M, Lanier L L. Immunol Rev 2010; 235:267-85; Jamieson A M, Diefenbach A, McMahon C W, et al. Immunity 2002; 17:19-29).

MICA and MICB (MICA/B) are NKG2D ligands which can, in some instances, be upregulated in several human cancers. Further MICA/B are transmembrane proteins with MHC-like extracellular domains that do not associate with beta-2 microglobulin nor present antigens. The proteins can undergo proteolytic cleavage in a multistep process and thereafter, the soluble MICA/B can bind NKG2D on NK cells. In some cases, the soluble MICA/B is shed in the blood plasma and serum in subjects with cancer.

The present disclosure is directed to improved bispecific MICA/B and NKp46 antibodies and the use of such antibodies for use in therapy (e.g., cancer therapy).

### SUMMARY

In one aspect, the present disclosure provides a MICA/B antigen binding domain comprising a set of vhCDR1-3 and vlCDR1-3 from a variable heavy domain and variable light domain pair selected from the group including: (i) SEQ ID NOs: 240-242 for vhCDR1-3 and SEQ ID NOs: 244-246 for vlCDR1-3 of D94837_1E11_1 [MICA/B]_H0_D94837_1E11_1 [MICA/B]_L0, (ii) SEQ ID NOs: 248-250 for vhCDR1-3 and SEQ ID NOs: 252-254 for vlCDR1-3 of D94837_1E11_1 [MICA/B]_H1_D94837_1E11_1 [MICA/B]_L1, (iii) SEQ ID NOs: 256-258 for vhCDR1-3 and SEQ ID NOs: 260-262 for vlCDR1-3 of D94837_1E11_1 [MICA/B]_H1_D94837_1E11_1 [MICA/B]_L2, (iv) SEQ ID NOs: 264-266 for vhCDR1-3 and SEQ ID NOs: 268-270 for vlCDR1-3 of D94837_1E11_1 [MICA/B]_H2_D94837_1E11_1 [MICA/B]_L1, (v) SEQ ID NOs: 272-274 for vhCDR1-3 and SEQ ID NOs: 276-278 for v1CDR1-3 of D94837_E11_1 [MICA/B]_H2_D94837_1E11_1 [MICA/B]_L2, (vi) SEQ ID NOs: 280-282 for vhCDR1-3 and SEQ ID NOs: 284-286 for v1CDR1-3 of 2E5 [MICA/B]_H0_2E5 [MICA/B]_L0, (vii) SEQ ID NOs: 288-290 for vhCDR1-3 and SEQ ID NOs: 292-294 for v1CDR1-3 of 2E5 [MICA/B]_H1_2E5 [MICA/B]_L1, (viii) SEQ ID NOs: 296-298 for vhCDR1-3 and SEQ ID NOs: 300-302 for v1CDR1-3 of 2E5 [MICA/B]_H1_2E5 [MICA/B]_L2, (ix) SEQ ID NOs: 304-306 for vhCDR1-3 and SEQ ID NOs: 308-310 for v1CDR1-3 of 2E5 [MICA/B]_H2_2E5 [MICA/B]_L1, (x) SEQ ID NOs: 312-314 for vhCDR1-3 and SEQ ID NOs: 316-318 for v1CDR1-3 of 2E5 [MICA/B]_H2_2E5 [MICA/B]_L2, (xi) SEQ ID NOs: 320-322 for vhCDR1-3 and SEQ ID NOs: 324-326 for v1CDR1-3 of D94852_2E12 [MICA/B]_H0_D94852_2E12 [MICA/B]_L0, (xii) SEQ ID NOs: 328-330 for vhCDR1-3 and SEQ ID NOs: 332-334 for v1CDR1-3 of D94852_2E12 [MICA/B]_H1_D94852_2E12 [MICA/B]_L1, (xiii) SEQ ID NOs: 336-338 for vhCDR1-3 and SEQ ID NOs: 340-342 for v1CDR1-3 of D94852_2E12 [MICA/B]_H1_D94852_2E12 [MICA/B]_L2, (xiv) SEQ ID NOs: 344-346 for vhCDR1-3 and SEQ ID NOs: 348-350 for v1CDR1-3 of D94852_2E12 [MICA/B]_H2_D94852_2E12 [MICA/B]_L1, (xv) SEQ ID NOs: 352-354 for vhCDR1-3 and SEQ ID NOs: 356-358 for v1CDR1-3 of D94852_2E12 [MICA/B]_H2_D94852_2E12 [MICA/B]_L2, (xvi) SEQ ID NOs: 360-362 for vhCDR1-3 and SEQ ID NOs: 364-366 for v1CDR1-3 of D99136_2F7 [MICA/B]_H0_D99136_2F7 [MICA/B]_L0, (xvii) SEQ ID NOs: 368-370 for vhCDR1-3 and SEQ ID NOs: 372-374 for v1CDR1-3 of D99136_2F7 [MICA/B]_H1_D99136_2F7 [MICA/B]_L1, (xviii) SEQ ID NOs: 376-378 for vhCDR1-3 and SEQ ID NOs: 380-382 for v1CDR1-3 of D99136_2F7 [MICA/B]_H1_D99136_2F7 [MICA/B]_L2, (xix) SEQ ID NOs: 384-386 for vhCDR1-3 and SEQ ID NOs: 388-390 for v1CDR1-3 of D99136_2F7 [MICA/B]_H2_D99136_2F7 [MICA/B]_L1, (xx) SEQ ID NOs: 392-394 for vhCDR1-3 and SEQ ID NOs: 396-398 for v1CDR1-3 of D99136_2F7 [MICA/B]_H2_D99136_2F7 [MICA/B]_L2, (xxi) SEQ ID NOs: 400-402 for vhCDR1-3 and SEQ ID NOs: 404-406 for v1CDR1-3 of D103388_1C7 [MICA/B]_H0_D103388_1C7 [MICA/B]_L0, (xxii) SEQ ID NOs: 408-410 for vhCDR1-3 and SEQ ID NOs: 412-414 for v1CDR1-3 of D103388_1C7 [MICA/B]_H1_D103388_1C7 [MICA/B]_L1, (xxiii) SEQ ID NOs: 416-418 for vhCDR1-3 and SEQ ID NOs: 420-422 for v1CDR1-3 of D103388_1C7 [MICA/B]_H1_D103388_1C7 [MICA/B]_L2, (xxiv) SEQ ID NOs: 424-426 for vhCDR1-3 and SEQ ID NOs: 428-430 for v1CDR1-3 of D103388_1C7 [MICA/B]_H2_D103388_1C7 [MICA/B]_L1, (xxv) SEQ ID NOs: 432-434 for vhCDR1-3 and SEQ ID NOs: 436-438 for v1CDR1-3 of D103388_1C7 [MICA/B]_H2_D103388_1C7 [MICA/B]_L2, (xxvi) SEQ ID NOs: 440-442 for vhCDR1-3 and SEQ ID NOs: 444-446 for v1CDR1-3 of D103388_1D7 [MICA/B]_H0_D103388_1D7 [MICA/B]_L0, (xxvii) SEQ ID NOs: 448-450 for vhCDR1-3 and SEQ ID NOs: 452-454 for v1CDR1-3 of D103388_1D7 [MICA/B]_H1_D103388_1D7 [MICA/B]_L1, (xxviii) SEQ ID NOs: 456-458 for vhCDR1-3 and SEQ ID NOs: 460-462 for v1CDR1-3 of D103388_1D7 [MICA/B]_H1_D103388_1D7 [MICA/B]_L2, (xxix) SEQ ID NOs: 464-466 for vhCDR1-3 and SEQ ID NOs: 468-470 for v1CDR1-3 of D103388_1D7 [MICA/B]_H2_D103388_1D7 [MICA/B]_L1, (xxx) SEQ ID NOs: 472-474 for vhCDR1-3 and SEQ ID NOs: 476-478 for v1CDR1-3 of D103388_1D7 [MICA/B]_H2_D103388_1D7 [MICA/B]_L2, (xxxi) SEQ ID NOs: 480-482 for vhCDR1-3 and SEQ ID NOs: 484-486 for v1CDR1-3 of D105317_1A2 [MICA/B]_H0_D105317_1A2 [MICA/B]_L0, (xxxii) SEQ ID NOs: 488-490 for vhCDR1-3 and SEQ ID NOs: 492-494 for v1CDR1-3 of D105317_1A2 [MICA/B]_H1_D105317_1A2 [MICA/B]_L1, (xxxiii) SEQ ID NOs: 496-498 for vhCDR1-3 and SEQ ID NOs: 500-502 for v1CDR1-3 of D105317_1A2 [MICA/B]_H1_D105317_1A2 [MICA/B]_L2, (xxxiv) SEQ ID NOs: 504-506 for vhCDR1-3 and SEQ ID NOs: 508-510 for v1CDR1-3 of D105317_1A2 [MICA/B]_H2_D105317_1A2 [MICA/B]_L1, (xxxv) SEQ ID NOs: 512-514 for vhCDR1-3 and SEQ ID NOs: 516-518 for vlCDR1-3 of D105317_1A2 [MICA/B]_H2_D105317_1A2 [MICA/B]_L2, (xxxvi) SEQ ID NOs: 520-522 for vhCDR1-3 and SEQ ID NOs: 524-526 for vlCDR1-3 of D99136_2C11 [MICA/B]_H0_D99i36_2C11 [MICA/B]_L0, (xxxvii) SEQ ID NOs: 528-530 for vhCDR1-3 and SEQ ID NOs: 532-534 for v1CDR1-3 of D99136_2C11 [MICA/B]_H1_D99136_2C11 [MICA/B]_L1, (xxxviii) SEQ ID NOs: 536-538 for vhCDR1-3 and SEQ ID NOs: 540-542 for v1CDR1-3 of D99136_2C11 [MICA/B]_H1_D99136_2C11 [MICA/B]_L2, (xxxix) SEQ ID NOs: 544-546 for vhCDR1-3 and SEQ ID NOs: 548-550 for v1CDR1-3 of D99136_2C11 [MICA/B]_H2_D99136_2C11 [MICA/B]_L1, (xl) SEQ ID NOs: 552-554 for vhCDR1-3 and SEQ ID NOs: 556-558 for vlCDR1-3 of D99136_2C11 [MICA/B]_H2_D99136_2C11 [MICA/B]_L2, (xli) SEQ ID NOs: 560-562 for vhCDR1-3 and SEQ ID NOs: 564-566 for vlCDR1-3 of D103388_1B11-2 [MICA/B]_H0_D103388_1B11-2 [MICA/B]_L0, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, and (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, as depicted in FIG. 21.

In another aspect, the present disclosure provides a MICA/B antigen binding domain comprising a variable heavy domain and variable light domain pair selected from the group including: (i) SEQ ID Nos: 239 and 243 for D94837_E11_1 [MICA/B]_H0_D94837_1E11_1 [MICA/B]_L0, (ii) SEQ ID Nos: 247 and 251 for D94837_1E11_1 [MICA/B]_H1_D94837_1E11_1 [MICA/B]_L1, (iii) SEQ ID Nos: 255 and 259 for D94837_1E11_1 [MICA/B]_H1_D94837_E11_1 [MICA/B]_L2, (iv) SEQ ID Nos: 263 and 267 for D94837_1E11_1 [MICA/B]_H2_D94837_E11_1 [MICA/B]_L1, (v) SEQ ID Nos: 271 and 275 for D94837_E11_1 [MICA/B]_H2_D94837_E11_1 [MICA/B]_L2, (vi) SEQ ID Nos: 279 and 283 for 2E5 [MICA/B]_H0_2E5 [MICA/B]_L0, (vii) SEQ ID Nos: 287 and 291 for 2E5 [MICA/B]_H1_2E5 [MICA/B]_L1, (viii) SEQ ID Nos: 295 and 299 for 2E5 [MICA/B]_H1_2E5 [MICA/B]_L2, (ix) SEQ ID Nos: 303 and 307 for 2E5 [MICA/B]_H2_2E5 [MICA/B]_L1, (x) SEQ ID Nos: 311 and 315 for 2E5 [MICA/B]_H2_2E5 [MICA/B]_L2, (xi) SEQ ID Nos: 319 and 323 for D94852_2E12 [MICA/B]_H0_D94852_2E12 [MICA/B]_L0, (xii) SEQ ID Nos: 327 and 331 for D94852_2E12 [MICA/B]_H1_D94852_2E12 [MICA/B]_L1, (xiii) SEQ ID Nos: 335 and 339 for D94852_2E12 [MICA/B]_H1_D94852_2E12 [MICA/B]_L2, (xiv) SEQ ID Nos: 343 and 347 for D94852_2E12 [MICA/B]_H2_D94852_2E12 [MICA/B]_L1, (xv) SEQ ID Nos: 351 and 355 for D94852_2E12 [MICA/B]_H2_D94852_2E12 [MICA/B]_L2, (xvi) SEQ ID Nos: 359 and 363 for D99136_2F7 [MICA/B]_H0_D99136_2F7 [MICA/B]_L0, (xvii) SEQ ID Nos: 367 and 371 for D99136_2F7 [MICA/B]_H1_D99136_2F7 [MICA/B]_L1, (xviii) SEQ ID Nos: 375 and 379 for D99136_2F7 [MICA/B]_H1_D99136_2F7 [MICA/B]_L2, (xix) SEQ ID Nos: 383 and 387 for D99136_2F7 [MICA/B]_H2_D99136_2F7 [MICA/B]_L1, (xx) SEQ ID Nos: 391 and 395 for D99136_2F7 [MICA/B]_H2_D99136_2F7 [MICA/B]_L2, (xxi) SEQ ID Nos: 399 and 403 for D103388_1C7 [MICA/B]_H0_D103388_1C7 [MICA/B]_L0, (xxii) SEQ ID Nos: 407 and 411 for D103388_1C7 [MICA/B]_H1_D103388_1C7 [MICA/B]_L1, (xxiii) SEQ ID Nos: 415 and 419 for D103388_1C7 [MICA/B]_H1_D103388_1C7 [MICA/B]_L2, (xxiv) SEQ ID Nos: 423 and 427 for D103388_1C7 [MICA/B]_H2_D103388_1C7 [MICA/B]_L1, (xxv) SEQ ID Nos: 431 and 435 for D103388_1C7 [MICA/B]_H2_D103388_1C7 [MICA/B]_L2, (xxvi) SEQ ID Nos: 439 and 443 for D103388_1D7 [MICA/B]_H0_D103388_1D7 [MICA/B]_L0, (xxvii) SEQ ID Nos: 447 and 451 for D103388_1D7 [MICA/B]_H1_D103388_1D7 [MICA/B]_L1, (xxviii) SEQ ID Nos: 455 and 459 for D103388_1D7 [MICA/B]_H1_D103388_1D7 [MICA/B]_L2, (xxix) SEQ ID Nos: 463 and 467 for D103388_1D7 [MICA/B]_H2_D103388_1D7 [MICA/B]_L1, (xxx) SEQ ID Nos: 471 and 475 for D103388_1D7 [MICA/B]_H2_D103388_1D7 [MICA/B]_L2, (xxxi) SEQ ID Nos: 479 and 483 for D105317_1A2 [MICA/B]_H0_D105317_1A2 [MICA/B]_L0, (xxxii) SEQ ID Nos: 487 and 491 for D105317_1A2 [MICA/B]_H1_D105317_1A2 [MICA/B]_L1, (xxxiii) SEQ ID Nos: 495 and 499 for D105317_1A2 [MICA/B]_H1_D105317_1A2 [MICA/B]_L2, (xxxiv) SEQ ID Nos: 503 and 507 for D105317_1A2 [MICA/B]_H2_D105317_1A2 [MICA/B]_L1, (xxxv) SEQ ID Nos: 511 and 515 for D105317_1A2 [MICA/B]_H2_D105317_1A2 [MICA/B]_L2, (xxxvi) SEQ ID Nos: 519 and 523 for D99136_2C11 [MICA/B]_H0_D99136_2C11 [MICA/B]_L0, (xxxvii) SEQ ID Nos: 527 and 531 for D99136_2C11 [MICA/B]_H1_D99136_2C11 [MICA/B]_L1, (xxxviii) SEQ ID Nos: 535 and 539 for D99136_2C11 [MICA/B]_H1_D99136_2C11 [MICA/B]_L2, (xxxix) SEQ ID Nos: 543 and 547 for D99136_2C11 [MICA/B]_H2_D99136_2C11 [MICA/B]_L1, (xl) SEQ ID Nos: 551 and 555 for D99136_2C11 [MICA/B]_H2_D99136_2C11 [MICA/B]_L2, (xli) SEQ ID Nos: 559 and 563 for D103388_1B11-2 [MICA/B]_H0_D103388_1B11-2 [MICA/B]_L0, (xlii) SEQ ID Nos: 567 and 571 for D105317_1B6 [MICA/B]_H0_D105317_1B6 [MICA/B]_L0, (xliii) SEQ ID Nos: 575 and 579 for D105317_1C8 [MICA/B]_H0_D105317_1C8 [MICA/B]_L0, (xliv) SEQ ID Nos: 583 and 587 for D105317_1F6 [MICA/B]_H0_D105317_1F6 [MICA/B]_L0, (xlv) SEQ ID Nos: 591 and 595 for D105317_1F7 [MICA/B]_H0_D105317_1F7 [MICA/B]_L0, (xlvi) SEQ ID Nos: 599 and 603 for D94837_1D3 [MICA/B]_H0_D94837_1D3 [MICA/B]_L0, (xlvii) SEQ ID Nos: 607 and 611 for D94837_1D8 [MICA/B]_H0_D94837_1D8 [MICA/B]_L0, (xlviii) SEQ ID Nos: 615 and 619 for D94837_1D9 [MICA/B]_H0_D94837_1D9 [MICA/B]_L0, (xlix) SEQ ID Nos: 623 and 627 for D94837_1E1 [MICA/B]_H0_D94837_1E1 [MICA/B]_L0, (1) SEQ ID Nos: 631 and 635 for D94852_2D4 [MICA/B]_H0_D94852_2D4 [MICA/B]_L0, (li) SEQ ID Nos: 639 and 643 for D94852_2G8 [MICA/B]_H0_D94852_2G8 [MICA/B]_L0, (lii) SEQ ID Nos: 647 and 651 for D88487_2A8 [MICA/B]_H0_D88487_2A8 [MICA/B]_L0, (liii) SEQ ID Nos: 655 and 659 for D94837_3B12 [MICA/B]_H0_D94837_3B12 [MICA/B]_L0, (liv) SEQ ID Nos: 663 and 667 for D99122_1H7 [MICA/B]_H0_D99122_1H7 [MICA/B]_L0, (lv) SEQ ID Nos: 671 and 675 for D99136_2E8 [MICA/B]_H0_D99136_2E8 [MICA/B]_L0, (lvi) SEQ ID Nos: 647 and 651 for D88487_2A8 [MICA/B]_H0_D88487_2A8 [MICA/B]_L0, (lvii) SEQ ID Nos: 679 and 683 for 3F9[MICA/B]_H0_3F9[MICA/B]_L0, (lviii) SEQ ID Nos: 687 and 691 for 6E1[MICA/B]_H0_6E1[MICA/B]_L0, (lix) SEQ ID Nos: 695 and 699 for 7C6[MICA/B]_H0_7C6[MICA/B]_L0, (lx) SEQ ID Nos: 703 and 707 for 13A9 [MICA/B]_H0_13A9 [MICA/B]_L0, and (lxi) SEQ ID Nos: 711 and 715 for 1D5 [MICA/B]_H0_1D5 [MICA/B]_L0, as depicted in FIG. 21.

In another aspect, the present disclosure provides an antibody comprising a MICA/B antigen binding domain (as described above).

In a further embodiment and in accordance with the above, the antibody is a monoclonal antibody or a bispecific antibody.

In another aspect, the present disclosure provides a composition comprising a MICA/B antigen binding domain or an antibody (as described above).

In another aspect, the present disclosure provides a nucleic acid composition comprising: (a) a first nucleic acid encoding a variable heavy domain (as described above), and (b) a second nucleic acid encoding a variable light domain (as described above).

In another aspect, the present disclosure provides an expression vector composition comprising: (a) a first expression vector comprising a first nucleic acid (as described above), and (b) a second expression vector comprising a second nucleic acid (as described above).

In another aspect, the present disclosure provides a host cell comprising an expression vector composition (as described above).

In another aspect, the present disclosure provides a method of making a MICA/B antigen binding domain or an antibody comprising such, the method comprising: (a) culturing a host cell (as described above) under conditions wherein the MICA/B antigen binding domain or the antibody comprising such is expressed, and (b) recovering the MICA/B antigen binding domain or the antibody comprising such.

In another aspect, the present disclosure provides a NKp46 antigen binding domain comprising a set of vhCDR1-3 and vlCDR1-3 from a variable heavy domain and variable light domain pair selected from the group including: SEQ ID NOs: 720-722 for vhCDR1-3 and SEQ ID NOs: 724-726 for vlCDR1-3 of 2C10A3.372[NKp46]_H1_2C10A3.372[NKp46]_L1, as depicted in FIG. 23.

In another aspect, the present disclosure provides a NKp46 antigen binding domain comprising a variable heavy domain and variable light domain pair selected from the group including: SEQ ID Nos: 719 and 723 for 2C10A3.372[NKp46]_H1_2C10A3.372[NKp46]_L1, as depicted in FIG. 23.

In another aspect, the present disclosure provides an antibody comprising a NKp46 antigen binding domain (as described above).

In a further embodiment and in accordance with the above, the antibody is a monoclonal antibody or a bispecific antibody.

In another aspect, the present disclosure provides a composition comprising a NKp46 antigen binding domain or an antibody (as described above).

In another aspect, the present disclosure provides a nucleic acid composition comprising: (a) a first nucleic acid encoding a variable heavy domain (as described above), and (b) a second nucleic acid encoding a variable light domain (as described above).

In another aspect, the present disclosure provides an expression vector composition comprising: (a) a first expression vector comprising a first nucleic acid (as described above), and (b) a second expression vector comprising a second nucleic acid (as described above).

In another aspect, the present disclosure provides a host cell comprising an expression vector composition (as described above).

In another aspect, the present disclosure provides a method of making a NKp46 antigen binding domain or an antibody comprising such, the method comprising: (a) culturing a host cell (as described above) under conditions wherein the NKp46 antigen binding domain or the antibody comprising such is expressed, and (b) recovering the NKp46 antigen binding domain or the antibody comprising such.

In another aspect, the present disclosure provides a heterodimeric antibody, comprising: (a) a first monomer, comprising: (i) an anti-NKp46 scFv comprising a first variable heavy VH1 domain, an scFv linker, and a first variable light VL1 domain; and (ii) a first Fc domain, wherein the scFv is covalently attached to the N-terminus of the first Fc domain using a domain linker; (b) a second monomer comprising a VH2-CH1-hinge-CH2-CH3 monomer, wherein VH2 is a second variable heavy domain and CH2-CH3 is a second Fc domain; and (c) a light chain comprising a second variable light VL2 domain, wherein the second variable heavy VH2 domain and the second variable light VL2 domain form a MICA/B antigen binding domain.

In a further embodiment and in accordance with the above, the MICA/B antigen binding domain comprises a set of vhCDR1-3 and vlCDR1-3 from a variable heavy domain and variable light domain pair selected from the group including: (i) SEQ ID NOs: 240-242 for vhCDR1-3 and SEQ ID NOs: 244-246 for vlCDR1-3 of D94837_E11_1 [MICA/B]_H0_D94837_1E11_1 [MICA/B]_L0, (ii) SEQ ID NOs: 248-250 for vhCDR1-3 and SEQ ID NOs: 252-254 for vlCDR1-3 of D94837_E11_1 [MICA/B]_H1_D94837_1E11_1 [MICA/B]_L1, (iii) SEQ ID NOs: 256-258 for vhCDR1-3 and SEQ ID NOs: 260-262 for vlCDR1-3 of D94837_1E11_1 [MICA/B]_H1_D94837_E11_1 [MICA/B]_L2, (iv) SEQ ID NOs: 264-266 for vhCDR1-3 and SEQ ID NOs: 268-270 for vlCDR1-3 of D94837_E11_1 [MICA/B]_H2_D94837_1E11_1 [MICA/B]_L1, (v) SEQ ID NOs: 272-274 for vhCDR1-3 and SEQ ID NOs: 276-278 for vlCDR1-3 of D94837_E11_1 [MICA/B]_H2_D94837_1E11_1 [MICA/B]_L2, (vi) SEQ ID NOs: 280-282 for vhCDR1-3 and SEQ ID NOs: 284-286 for vlCDR1-3 of 2E5 [MICA/B]_H0_2E5 [MICA/B]_L0, (vii) SEQ ID NOs: 288-290 for vhCDR1-3 and SEQ ID NOs: 292-294 for vlCDR1-3 of 2E5 [MICA/B]_H1_2E5 [MICA/B]_L1, (viii) SEQ ID NOs: 296-298 for vhCDR1-3 and SEQ ID NOs: 300-302 for vlCDR1-3 of 2E5 [MICA/B]_H1_2E5 [MICA/B]_L2, (ix) SEQ ID NOs: 304-306 for vhCDR1-3 and SEQ ID NOs: 308-310 for vlCDR1-3 of 2E5 [MICA/B]_H2_2E5 [MICA/B]_L1, (x) SEQ ID NOs: 312-314 for vhCDR1-3 and SEQ ID NOs: 316-318 for vlCDR1-3 of 2E5 [MICA/B]_H2_2E5 [MICA/B]_L2, (xi) SEQ ID NOs: 320-322 for vhCDR1-3 and SEQ ID NOs: 324-326 for vlCDR1-3 of D94852_2E12 [MICA/B]_H0_D94852_2E12 [MICA/B]_L0, (xii) SEQ ID NOs: 328-330 for vhCDR1-3 and SEQ ID NOs: 332-334 for vlCDR1-3 of D94852_2E12 [MICA/B]_H1_D94852_2E12 [MICA/B]_L1, (xiii) SEQ ID NOs: 336-338 for vhCDR1-3 and SEQ ID NOs: 340-342 for vlCDR1-3 of D94852_2E12 [MICA/B]_H1_D94852_2E12 [MICA/B]_L2, (xiv) SEQ ID NOs: 344-346 for vhCDR1-3 and SEQ ID NOs: 348-350 for vlCDR1-3 of D94852_2E12 [MICA/B]_H2_D94852_2E12 [MICA/B]_L1, (xv) SEQ ID NOs: 352-354 for vhCDR1-3 and SEQ ID NOs: 356-358 for vlCDR1-3 of D94852_2E12 [MICA/B]_H2_D94852_2E12 [MICA/B]_L2, (xvi) SEQ ID NOs: 360-362 for vhCDR1-3 and SEQ ID NOs: 364-366 for vlCDR1-3 of D99136_2F7 [MICA/B]_H0_D99136_2F7 [MICA/B]_L0, (xvii) SEQ ID NOs: 368-370 for vhCDR1-3 and SEQ ID NOs: 372-374 for vlCDR1-3 of D99136_2F7 [MICA/B]_H1_D99136_2F7 [MICA/B]_L1, (xviii) SEQ ID NOs: 376-378 for vhCDR1-3 and SEQ ID NOs: 380-382 for vlCDR1-3 of D99136_2F7 [MICA/B]_H1_D99136_2F7 [MICA/B]_L2, (xix) SEQ ID NOs: 384-386 for vhCDR1-3 and SEQ ID NOs: 388-390 for vlCDR1-3 of D99136_2F7 [MICA/B]_H2_D99136_2F7 [MICA/B]_L1, (xx) SEQ ID NOs: 392-394 for vhCDR1-3 and SEQ ID NOs: 396-398 for v1CDR1-3 of D99136_2F7 [MICA/B]_H2_D99136_2F7 [MICA/B]_L2, (xxi) SEQ ID NOs: 400-402 for vhCDR1-3 and SEQ ID NOs: 404-406 for v1CDR1-3 of D103388_1C7 [MICA/B]_H0_D103388_1C7 [MICA/B]_L0, (xxii) SEQ ID NOs: 408-410 for vhCDR1-3 and SEQ ID NOs: 412-414 for v1CDR1-3 of D103388_1C7 [MICA/B]_H1_D103388_1C7 [MICA/B]_L1, (xxiii) SEQ ID NOs: 416-418 for vhCDR1-3 and SEQ ID NOs: 420-422 for v1CDR1-3 of D103388_1C7 [MICA/B]_H1_D103388_1C7 [MICA/B]_L2, (xxiv) SEQ ID NOs: 424-426 for vhCDR1-3 and SEQ ID NOs: 428-430 for v1CDR1-3 of D103388_1C7 [MICA/B]_H2_D103388_1C7 [MICA/B]_L1, (xxv) SEQ ID NOs: 432-434 for vhCDR1-3 and SEQ ID NOs: 436-438 for v1CDR1-3 of D103388_1C7 [MICA/B]_H2_D103388_1C7 [MICA/B]_L2, (xxvi) SEQ ID NOs: 440-442 for vhCDR1-3 and SEQ ID NOs: 444-446 for v1CDR1-3 of D103388_1D7 [MICA/B]_H0_D103388_1D7 [MICA/B]_L0, (xxvii) SEQ ID NOs: 448-450 for vhCDR1-3 and SEQ ID NOs: 452-454 for v1CDR1-3 of D103388_1D7 [MICA/B]_H1_D103388_1D7 [MICA/B]_L1, (xxviii) SEQ ID NOs: 456-458 for vhCDR1-3 and SEQ ID NOs: 460-462 for v1CDR1-3 of D103388_1D7 [MICA/B]_H1_D103388_1D7 [MICA/B]_L2, (xxix) SEQ ID NOs: 464-466 for vhCDR1-3 and SEQ ID NOs: 468-470 for v1CDR1-3 of D103388_1D7 [MICA/B]_H2_D103388_1D7 [MICA/B]_L1, (xxx) SEQ ID NOs: 472-474 for vhCDR1-3 and SEQ ID NOs: 476-478 for v1CDR1-3 of D103388_1D7 [MICA/B]_H2_D103388_1D7 [MICA/B]_L2, (xxxi) SEQ ID NOs: 480-482 for vhCDR1-3 and SEQ ID NOs: 484-486 for v1CDR1-3 of D105317_1A2 [MICA/B]_H0_D105317_1A2 [MICA/B]_L0, (xxxii) SEQ ID NOs: 488-490 for vhCDR1-3 and SEQ ID NOs: 492-494 for v1CDR1-3 of D105317_1A2 [MICA/B]_H1_D105317_1A2 [MICA/B]_L1, (xxxiii) SEQ ID NOs: 496-498 for vhCDR1-3 and SEQ ID NOs: 500-502 for v1CDR1-3 of D105317_1A2 [MICA/B]_H1_D105317_1A2 [MICA/B]_L2, (xxxiv) SEQ ID NOs: 504-506 for vhCDR1-3 and SEQ ID NOs: 508-510 for v1CDR1-3 of D105317_1A2 [MICA/B]_H2_D105317_1A2 [MICA/B]_L1, (xxxv) SEQ ID NOs: 512-514 for vhCDR1-3 and SEQ ID NOs: 516-518 for vlCDR1-3 of D105317_1A2 [MICA/B]_H2_D105317_1A2 [MICA/B]_L2, (xxxvi) SEQ ID NOs: 520-522 for vhCDR1-3 and SEQ ID NOs: 524-526 for vlCDR1-3 of D99136_2C11 [MICA/B]_H0_D99136_2C11 [MICA/B]_L0, (xxxvii) SEQ ID NOs: 528-530 for vhCDR1-3 and SEQ ID NOs: 532-534 for v1CDR1-3 of D99136_2C11 [MICA/B]_H1_D99136_2C11 [MICA/B]_L1, (xxxviii) SEQ ID NOs: 536-538 for vhCDR1-3 and SEQ ID NOs: 540-542 for v1CDR1-3 of D99136_2C11 [MICA/B]_H1_D99136_2C11 [MICA/B]_L2, (xxxix) SEQ ID NOs: 544-546 for vhCDR1-3 and SEQ ID NOs: 548-550 for v1CDR1-3 of D99136_2C11 [MICA/B]_H2_D99136_2C11 [MICA/B]_L1, (xl) SEQ ID NOs: 552-554 for vhCDR1-3 and SEQ ID NOs: 556-558 for v1CDR1-3 of D99136_2C11 [MICA/B]_H2_D99136_2C11 [MICA/B]_L2, (xli) SEQ ID NOs: 560-562 for vhCDR1-3 and SEQ ID NOs: 564-566 for v1CDR1-3 of D103388_1B11-2 [MICA/B]_H0_D103388_1B11-2 [MICA/B]_L0, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (lvii) SEQ ID NOs: 680-682 for vhCDR1-3 and SEQ ID NOs: 684-686 for vlCDR1-3 of 3F9[MICA/B]_H0_3F9[MICA/B]_L0, (lviii) SEQ ID NOs: 688-690 for vhCDR1-3 and SEQ ID NOs: 692-694 for vlCDR1-3 of 6E1[MICA/B]_H0_6E1[MICA/B]_L0, (lix) SEQ ID NOs: 696-698 for vhCDR1-3 and SEQ ID NOs: 700-702 for vlCDR1-3 of 7C6[MICA/B]_H0_7C6[MICA/B]_L0, (lx) SEQ ID NOs: 704-706 for vhCDR1-3 and SEQ ID NOs: 708-710 for vlCDR1-3 of 13A9 [MICA/B]_H0_13A9 [MICA/B]_L0, and (lxi) SEQ ID NOs: 712-714 for vhCDR1-3 and SEQ ID NOs: 716-718 for vlCDR1-3 of 1D5 [MICA/B]_H0_1D5 [MICA/B]_L0, as depicted in FIGS. 21 and 22.

In a further embodiment and in accordance with any of the above, the MICA/B antigen binding domain comprises a variable heavy domain and variable light domain pair selected from the group including: (i) SEQ ID Nos: 239 and 243 for D94837_1E11_1 [MICA/B]_H0_D94837_1E11_1 [MICA/B]_L0, (ii) SEQ ID Nos: 247 and 251 for D94837_1E11_l [MICA/B]_H1_D94837_1E1_1 [MICA/B]_L1, (iii) SEQ ID Nos: 255 and 259 for D94837_1E11_1 [MICA/B]_H1_D94837_1E11_1 [MICA/B]_L2, (iv) SEQ ID Nos: 263 and 267 for D94837_1E11_1 [MICA/B]_H2_D94837_E11_1 [MICA/B]_L1, (v) SEQ ID Nos: 271 and 275 for D94837_1E11_1 [MICA/B]_H2_D94837_1E11_1 [MICA/B]_L2, (vi) SEQ ID Nos: 279 and 283 for 2E5 [MICA/B]_H0_2E5 [MICA/B]_L0, (vii) SEQ ID Nos: 287 and 291 for 2E5 [MICA/B]_H1_2E5 [MICA/B]_L1, (viii) SEQ ID Nos: 295 and 299 for 2E5 [MICA/B]_H1_2E5 [MICA/B]_L2, (ix) SEQ ID Nos: 303 and 307 for 2E5 [MICA/B]_H2_2E5 [MICA/B]_L1, (x) SEQ ID Nos: 311 and 315 for 2E5 [MICA/B]_H2_2E5 [MICA/B]_L2, (xi) SEQ ID Nos: 319 and 323 for D94852_2E12 [MICA/B]_H0_D94852_2E12 [MICA/B]_L0, (xii) SEQ ID Nos: 327 and 331 for D94852_2E12 [MICA/B]_H1_D94852_2E12 [MICA/B]_L1, (xiii) SEQ ID Nos: 335 and 339 for D94852_2E12 [MICA/B]_H1_D94852_2E12 [MICA/B]_L2, (xiv) SEQ ID Nos: 343 and 347 for D94852_2E12 [MICA/B]_H2_D94852_2E12 [MICA/B]_L1, (xv) SEQ ID Nos: 351 and 355 for D94852_2E12 [MICA/B]_H2_D94852_2E12 [MICA/B]_L2, (xvi) SEQ ID Nos: 359 and 363 for D99136_2F7 [MICA/B]_H0_D99136_2F7 [MICA/B]_L0, (xvii) SEQ ID Nos: 367 and 371 for D99136_2F7 [MICA/B]_H1_D99136_2F7 [MICA/B]_L1, (xviii) SEQ ID Nos: 375 and 379 for D99136_2F7 [MICA/B]_H1_D99136_2F7 [MICA/B]_L2, (xix) SEQ ID Nos: 383 and 387 for D99136_2F7 [MICA/B]_H2_D99136_2F7 [MICA/B]_L1, (xx) SEQ ID Nos: 391 and 395 for D99136_2F7 [MICA/B]_H2_D99136_2F7 [MICA/B]_L2, (xxi) SEQ ID Nos: 399 and 403 for D103388_1C7 [MICA/B]_H0_D103388_1C7 [MICA/B]_L0, (xxii) SEQ ID Nos: 407 and 411 for D103388_1C7 [MICA/B]_H1_D103388_1C7 [MICA/B]_L1, (xxiii) SEQ ID Nos: 415 and 419 for D103388_1C7 [MICA/B]_H1_D103388_1C7 [MICA/B]_L2, (xxiv) SEQ ID Nos: 423 and 427 for D103388_1C7 [MICA/B]_H2_D103388_1C7 [MICA/B]_L1, (xxv) SEQ ID Nos: 431 and 435 for D103388_1C7 [MICA/B]_H2_D103388_1C7 [MICA/B]_L2, (xxvi) SEQ ID Nos: 439 and 443 for D103388_1D7 [MICA/B]_H0_D103388_1D7 [MICA/B]_L0, (xxvii) SEQ ID Nos: 447 and 451 for D103388_1D7 [MICA/B]_H1_D103388_1D7 [MICA/B]_L1, (xxviii) SEQ ID Nos: 455 and 459 for D103388_1D7 [MICA/B]_H1D103388_1D7 [MICA/B]_L2, (xxix) SEQ ID Nos: 463 and 467 for D103388_1D7 [MICA/B]_H2_D103388_1D7 [MICA/B]_L1, (xxx) SEQ ID Nos: 471 and 475 for D103388_1D7 [MICA/B]_H2_D103388_1D7 [MICA/B]_L2, (xxxi) SEQ ID Nos: 479 and 483 for D105317_1A2 [MICA/B]_H0_D105317_1A2 [MICA/B]_L0, (xxxii) SEQ ID Nos: 487 and 491 for D105317_1A2 [MICA/B]_H1_D105317_1A2 [MICA/B]_L1, (xxxiii) SEQ ID Nos: 495 and 499 for D105317_1A2 [MICA/B]_H1_D105317_1A2 [MICA/B]_L2, (xxxiv) SEQ ID Nos: 503 and 507 for D105317_1A2 [MICA/B]_H2_D105317_1A2 [MICA/B]_L1, (xxxv) SEQ ID Nos: 511 and 515 for D105317_1A2 [MICA/B]_H2_D105317_1A2 [MICA/B]_L2, (xxxvi) SEQ ID Nos: 519 and 523 for D99136_2C11 [MICA/B]_H0_D99136_2C11 [MICA/B]_L0, (xxxvii) SEQ ID Nos: 527 and 531 for D99136_2C11 [MICA/B]_H1_D99136_2C11 [MICA/B]_L1, (xxxviii) SEQ ID Nos: 535 and 539 for D99136_2C11 [MICA/B]_H1_D99136_2C11 [MICA/B]_L2, (xxxix) SEQ ID Nos: 543 and 547 for D99136_2C11 [MICA/B]_H2_D99136_2C11 [MICA/B]_L1, (xl) SEQ ID Nos: 551 and 555 for D99136_2C11 [MICA/B]_H2_D99136_2C11 [MICA/B]_L2, (xli) SEQ ID Nos: 559 and 563 for D103388_1B11-2 [MICA/B]_H0_D103388_1B11-2 [MICA/B]_L0, (xlii) SEQ ID Nos: 567 and 571 for D105317_1B6 [MICA/B]_H0_D105317_1B6 [MICA/B]_L0, (xliii) SEQ ID Nos: 575 and 579 for D105317_1C8 [MICA/B]_H0_D105317_1C8 [MICA/B]_L0, (xliv) SEQ ID Nos: 583 and 587 for D105317_1F6 [MICA/B]_H0_D105317_1F6 [MICA/B]_L0, (xlv) SEQ ID Nos: 591 and 595 for D105317_1F7 [MICA/B]_H0_D105317_1F7 [MICA/B]_L0, (xlvi) SEQ ID Nos: 599 and 603 for D94837_1D3 [MICA/B]_H0_D94837_1D3 [MICA/B]_L0, (xlvii) SEQ ID Nos: 607 and 611 for D94837_1D8 [MICA/B]_H0_D94837_1D8 [MICA/B]_L0, (xlviii) SEQ ID Nos: 615 and 619 for D94837_1D9 [MICA/B]_H0_D94837_1D9 [MICA/B]_L0, (xlix) SEQ ID Nos: 623 and 627 for D94837_1E1 [MICA/B]_H0_D94837_1E1 [MICA/B]_L0, (1) SEQ ID Nos: 631 and 635 for D94852_2D4 [MICA/B]_H0_D94852_2D4 [MICA/B]_L0, (li) SEQ ID Nos: 639 and 643 for D94852_2G8 [MICA/B]_H0_D94852_2G8 [MICA/B]_L0, (lii) SEQ ID Nos: 647 and 651 for D88487_2A8 [MICA/B]_H0_D88487_2A8 [MICA/B]_L0, (liii) SEQ ID Nos: 655 and 659 for D94837_3B12 [MICA/B]_H0_D94837_3B12 [MICA/B]_L0, (liv) SEQ ID Nos: 663 and 667 for D99122_1H7 [MICA/B]_H0_D99122_1H7 [MICA/B]_L0, (lv) SEQ ID Nos: 671 and 675 for D99136_2E8 [MICA/B]_H0_D99136_2E8 [MICA/B]_L0, (lvi) SEQ ID Nos: 647 and 651 for D88487_2A8 [MICA/B]_H0_D88487_2A8 [MICA/B]_L0, (lvii) SEQ ID Nos: 679 and 683 for 3F9[MICA/B]_H0_3F9[MICA/B]_L0, (lviii) SEQ ID Nos: 687 and 691 for 6E1[MICA/B]_H0_6E1[MICA/B]_L0, (lix) SEQ ID Nos: 695 and 699 for 7C6[MICA/B]_H0_7C6[MICA/B]_L0, (lx) SEQ ID Nos: 703 and 707 for 13A9 [MICA/B]_H0_13A9 [MICA/B]_L0, and (lxi) SEQ ID Nos: 711 and 715 for 1D5 [MICA/B]_H0_1D5 [MICA/B]_L0, as depicted in FIGS. 21 and 22.

In a further embodiment and in accordance with any of the above, the anti-NKp46 scFv comprises a set of vhCDR1-3 and vlCDR1-3 from a first variable heavy VH1 domain and first variable light VL1 domain pair selected from the group including: (i) SEQ ID NOs: 720-722 for vhCDR1-3 and SEQ ID NOs: 724-726 for vlCDR1-3 of 2C10A3.372[NKp46]_H1_2C10A3.372[NKp46]_L1, and (ii) SEQ ID NOs: 728-730 for vhCDR1-3 and SEQ ID NOs: 732-734 for vlCDR1-3 of NKp46-A[NKp46]_H_NKp46-A[NKp46]_L, as depicted in FIGS. 23 and 24.

In a further embodiment and in accordance with any of the above, the anti-NKp46 scFv comprises a variable heavy domain and variable light domain pair selected from the group including: (i) SEQ ID Nos: 719 and 723 for 2C10A3.372[NKp46]_H1_2C10A3.372[NKp46]_L1, and (ii) SEQ ID Nos: 727 and 731 for NKp46-A[NKp46]_H_NKp46-A[NKp46]_L, as depicted in FIGS. 23 and 24.

In a further embodiment and in accordance with any of the above, the first variable light VL1 domain of the anti-NKp46 scFv is covalently attached to the N-terminus of the first Fc domain using a domain linker, or the first variable heavy VH1 domain of the anti-NKp46 scFv is covalently attached to the N-terminus of the first Fc domain using a domain linker.

In a further embodiment and in accordance with any of the above, the heterodimeric antibody comprises a first amino acid sequence, a second amino acid sequence, and a third amino acid sequence, wherein the first, second, and third amino acid sequences are selected from the group including: (i) the amino acid sequences of SEQ ID NOs: 794-796 of XENP46810, (ii) the amino acid sequences of SEQ ID NOS: 797-799 of XENP46811, and (iii) the amino acid sequences of SEQ ID NOs: 800-802 of XENP46812, as depicted in FIG. 31.

In a further embodiment and in accordance with any of the above, the scFv linker is a charged scFv linker.

In a further embodiment and in accordance with the above, the scFv linker is a charged scFv linker having the amino acid sequence (GKPGS)₄ (SEQ ID NO: 1).

In a further embodiment and in accordance with any of the above, the first and second Fc domains are each variant Fc domains.

In a further embodiment and in accordance with the above, the first and/or second variant Fc domains comprise one or more FcγRIIIA (CD16a) binding variant substitutions.

In a further embodiment and in accordance with the above, the one or more FcγRIIIA (CD16a) binding variant substitutions are selected from the group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with the above, the first and second variant Fc domains comprise a set of FcγRIIIA (CD16a) binding variant substitutions selected from the group including: (i) S239D/I332E: S239D/I332E, (ii) S239D: S239D, (iii) 1332E: 1332E, (iv) WT: S239D/I332E, (v) WT: S239D, (vi) WT: 1332E, (vii) S239D/I332E: WT, (viii) S239D: WT, (ix) 1332E: WT, (x) S239D/I332E: S239D, (xi) S239D/I332E: 1332E, (xii) S239D: S239D/I332E, (xiii) 1332E: S239D/I332E, (xiv) S239D: 1332E, and (xv) 1332E: S239D, wherein numbering is according to EU numbering.

In a further embodiment and in accordance with any of the above, the first and/or second variant Fc domains comprise the FcγRIIIA (CD16a) binding variant substitutions of S239D/I332E, wherein numbering is according to EU numbering.

In a further embodiment and in accordance with any of the above, the first and second variant Fe domains comprise a set of heterodimerization variants selected from the group including those depicted in FIGS. 4A-4F, wherein numbering is according to EU numbering.

In a further embodiment and in accordance with the above, the set of heterodimerization variants is selected from the group including: (i) S364K/E357Q: L368D/K370S, (ii) S364K: L368D/K370S, (iii) S364K: L368E/K370S, (iv) D401K: T411E/K360E/Q362E, and (v) T366W: T366S/L368A/Y407V, wherein numbering is according to EU numbering.

In a further embodiment and in accordance with any of the above, the first and second variant Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant Fc domain comprises one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first monomer comprises amino acid variants S364K/E357Q/E233P/L234V/L235A/G236del/S267K, wherein the second monomer comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second monomers each further comprise amino acid variants M428L/N434S, M428L/434A, or M252Y/S254T/T256E, wherein numbering is according to EU numbering.

In another aspect, a nucleic acid composition comprising nucleic acids encoding the first and second monomers and the light chain of the antibody (as described above) is provided.

In another aspect, an expression vector comprising the nucleic acids (as described above) is provided.

In another aspect, a host cell transformed with the expression vector (as described above) is provided.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, the present disclosure provides a heterodimeric antibody, comprising: (a) a first monomer, comprising: (i) an anti-MICA/B scFv comprising a first variable heavy VH1 domain, an scFv linker, and a first variable light VL1 domain; and (ii) a first Fc domain, wherein the scFv is covalently attached to the N-terminus of the first Fc domain using a domain linker; (b) a second monomer comprising a VH2-CH1-hinge-CH2-CH3 monomer, wherein VH2 is a second variable heavy domain and CH2-CH3 is a second Fc domain; and (c) a light chain comprising a second variable light VL2 domain, wherein the second variable heavy VH2 domain and the second variable light VL2 domain form a NKp46 antigen binding domain.

In a further embodiment and in accordance with the above, the NKp46 antigen binding domain comprises a set of vhCDR1-3 and vlCDR1-3 from a variable heavy domain and variable light domain pair selected from the group including: (i) SEQ ID NOs: 720-722 for vhCDR1-3 and SEQ ID NOs: 724-726 for vlCDR1-3 of 2C10A3.372[NKp46]_H1_2C10A3.372[NKp46]_L1, and (ii) SEQ ID NOs: 728-730 for vhCDR1-3 and SEQ ID NOs: 732-734 for vlCDR1-3 of NKp46-A[NKp46]_H_NKp46-A[NKp46]_L, as depicted in FIGS. 23 and 24.

In a further embodiment and in accordance with any of the above, the NKp46 antigen binding domain comprises a variable heavy domain and variable light domain pair selected from the group including: (i) SEQ ID Nos: 719 and 723 for 2C10A3.372[NKp46]_H1_2C10A3.372[NKp46]_L1, and (ii) SEQ ID Nos: 727 and 731 for NKp46-A[NKp46]_H_NKp46-A[NKp46]_L, as depicted in FIGS. 23 and 24.

In a further embodiment and in accordance with any of the above, the anti-MICA/B scFv comprises a set of vhCDR1-3 and vlCDR1-3 from a first variable heavy VH1 domain and first variable light VL1 domain pair selected from the group including: (i) SEQ ID NOs: 240-242 for vhCDR1-3 and SEQ ID NOs: 244-246 for vlCDR1-3 of D94837_1E1_1 [MICA/B]_H0_D94837_E11_1 [MICA/B]_L0, (ii) SEQ ID NOs: 248-250 for vhCDR1-3 and SEQ ID NOs: 252-254 for v1CDR1-3 of D94837_1E11_1 [MICA/B]_H1_D94837_1E11_1 [MICA/B]_L1, (iii) SEQ ID NOs: 256-258 for vhCDR1-3 and SEQ ID NOs: 260-262 for v1CDR1-3 of D94837_E11_1 [MICA/B]_H1_D94837_1E11_1 [MICA/B]_L2, (iv) SEQ ID NOs: 264-266 for vhCDR1-3 and SEQ ID NOs: 268-270 for v1CDR1-3 of D94837_1E11_1 [MICA/B]_H2_D94837_1E11_1 [MICA/B]_L1, (v) SEQ ID NOs: 272-274 for vhCDR1-3 and SEQ ID NOs: 276-278 for v1CDR1-3 of D94837_1E11_1 [MICA/B]_H2_D94837_1E11_1 [MICA/B]_L2, (vi) SEQ ID NOs: 280-282 for vhCDR1-3 and SEQ ID NOs: 284-286 for v1CDR1-3 of 2E5 [MICA/B]_H0_2E5 [MICA/B]_L0, (vii) SEQ ID NOs: 288-290 for vhCDR1-3 and SEQ ID NOs: 292-294 for v1CDR1-3 of 2E5 [MICA/B]_H1_2E5 [MICA/B]_L1, (viii) SEQ ID NOs: 296-298 for vhCDR1-3 and SEQ ID NOs: 300-302 for v1CDR1-3 of 2E5 [MICA/B]_H1_2E5 [MICA/B]_L2, (ix) SEQ ID NOs: 304-306 for vhCDR1-3 and SEQ ID NOs: 308-310 for v1CDR1-3 of 2E5 [MICA/B]_H2_2E5 [MICA/B]_L1, (x) SEQ ID NOs: 312-314 for vhCDR1-3 and SEQ ID NOs: 316-318 for v1CDR1-3 of 2E5 [MICA/B]_H2_2E5 [MICA/B]_L2, (xi) SEQ ID NOs: 320-322 for vhCDR1-3 and SEQ ID NOs: 324-326 for v1CDR1-3 of D94852_2E12 [MICA/B]_H0_D94852_2E12 [MICA/B]_L0, (xii) SEQ ID NOs: 328-330 for vhCDR1-3 and SEQ ID NOs: 332-334 for v1CDR1-3 of D94852_2E12 [MICA/B]_H1_D94852_2E12 [MICA/B]_L1, (xiii) SEQ ID NOs: 336-338 for vhCDR1-3 and SEQ ID NOs: 340-342 for v1CDR1-3 of D94852_2E12 [MICA/B]_H1_D94852_2E12 [MICA/B]_L2, (xiv) SEQ ID NOs: 344-346 for vhCDR1-3 and SEQ ID NOs: 348-350 for v1CDR1-3 of D94852_2E12 [MICA/B]_H2_D94852_2E12 [MICA/B]_L1, (xv) SEQ ID NOs: 352-354 for vhCDR1-3 and SEQ ID NOs: 356-358 for v1CDR1-3 of D94852_2E12 [MICA/B]_H2_D94852_2E12 [MICA/B]_L2, (xvi) SEQ ID NOs: 360-362 for vhCDR1-3 and SEQ ID NOs: 364-366 for v1CDR1-3 of D99136_2F7 [MICA/B]_H0_D99136_2F7 [MICA/B]_L0, (xvii) SEQ ID NOs: 368-370 for vhCDR1-3 and SEQ ID NOs: 372-374 for v1CDR1-3 of D99136_2F7 [MICA/B]_H1_D99136_2F7 [MICA/B]_L1, (xviii) SEQ ID NOs: 376-378 for vhCDR1-3 and SEQ ID NOs: 380-382 for v1CDR1-3 of D99136_2F7 [MICA/B]_H1_D99136_2F7 [MICA/B]_L2, (xix) SEQ ID NOs: 384-386 for vhCDR1-3 and SEQ ID NOs: 388-390 for v1CDR1-3 of D99136_2F7 [MICA/B]_H2_D99136_2F7 [MICA/B]_L1, (xx) SEQ ID NOs: 392-394 for vhCDR1-3 and SEQ ID NOs: 396-398 for v1CDR1-3 of D99136_2F7 [MICA/B]_H2_D99136_2F7 [MICA/B]_L2, (xxi) SEQ ID NOs: 400-402 for vhCDR1-3 and SEQ ID NOs: 404-406 for v1CDR1-3 of D103388_1C7 [MICA/B]_H0_D103388_1C7 [MICA/B]_L0, (xxii) SEQ ID NOs: 408-410 for vhCDR1-3 and SEQ ID NOs: 412-414 for v1CDR1-3 of D103388_1C7 [MICA/B]_H1_D103388_1C7 [MICA/B]_L1, (xxiii) SEQ ID NOs: 416-418 for vhCDR1-3 and SEQ ID NOs: 420-422 for v1CDR1-3 of D103388_1C7 [MICA/B]_H1_D103388_1C7 [MICA/B]_L2, (xxiv) SEQ ID NOs: 424-426 for vhCDR1-3 and SEQ ID NOs: 428-430 for v1CDR1-3 of D103388_1C7 [MICA/B]_H2_D103388_1C7 [MICA/B]_L1, (xxv) SEQ ID NOs: 432-434 for vhCDR1-3 and SEQ ID NOs: 436-438 for v1CDR1-3 of D103388_1C7 [MICA/B]_H2_D103388_1C7 [MICA/B]_L2, (xxvi) SEQ ID NOs: 440-442 for vhCDR1-3 and SEQ ID NOs: 444-446 for v1CDR1-3 of D103388_1D7 [MICA/B]_H0_D103388_1D7 [MICA/B]_L0, (xxvii) SEQ ID NOs: 448-450 for vhCDR1-3 and SEQ ID NOs: 452-454 for v1CDR1-3 of D103388_1D7 [MICA/B]_H1_D103388_1D7 [MICA/B]_L1, (xxviii) SEQ ID NOs: 456-458 for vhCDR1-3 and SEQ ID NOs: 460-462 for v1CDR1-3 of D103388_1D7 [MICA/B]_H1_D103388_1D7 [MICA/B]_L2, (xxix) SEQ ID NOs: 464-466 for vhCDR1-3 and SEQ ID NOs: 468-470 for v1CDR1-3 of D103388_1D7 [MICA/B]_H2_D103388_1D7 [MICA/B]_L1, (xxx) SEQ ID NOs: 472-474 for vhCDR1-3 and SEQ ID NOs: 476-478 for v1CDR1-3 of D103388_1D7 [MICA/B]_H2_D103388_1D7 [MICA/B]_L2, (xxxi) SEQ ID NOs: 480-482 for vhCDR1-3 and SEQ ID NOs: 484-486 for v1CDR1-3 of D105317_1A2 [MICA/B]_H0_D105317_1A2 [MICA/B]_L0, (xxxii) SEQ ID NOs: 488-490 for vhCDR1-3 and SEQ ID NOs: 492-494 for v1CDR1-3 of D105317_1A2 [MICA/B]_H1_D105317_1A2 [MICA/B]_L1, (xxxiii) SEQ ID NOs: 496-498 for vhCDR1-3 and SEQ ID NOs: 500-502 for v1CDR1-3 of D105317_1A2 [MICA/B]_H1_D105317_1A2 [MICA/B]_L2, (xxxiv) SEQ ID NOs: 504-506 for vhCDR1-3 and SEQ ID NOs: 508-510 for vlCDR1-3 of D105317_1A2 [MICA/B]_H2_D105317_1A2 [MICA/B]_L1, (xxxv) SEQ ID NOs: 512-514 for vhCDR1-3 and SEQ ID NOs: 516-518 for v1CDR1-3 of D105317_1A2 [MICA/B]_H2_D105317_1A2 [MICA/B]_L2, (xxxvi) SEQ ID NOs: 520-522 for vhCDR1-3 and SEQ ID NOs: 524-526 for v1CDR1-3 of D99136_2C11 [MICA/B]_H0_D99136_2C11 [MICA/B]_L0, (xxxvii) SEQ ID NOs: 528-530 for vhCDR1-3 and SEQ ID NOs: 532-534 for vlCDR1-3 of D99136_2C11 [MICA/B]_H1_D99136_2C11 [MICA/B]_L1, (xxxviii) SEQ ID NOs: 536-538 for vhCDR1-3 and SEQ ID NOs: 540-542 for v1CDR1-3 of D99136_2C11 [MICA/B]_H1_D99136_2C11 [MICA/B]_L2, (xxxix) SEQ ID NOs: 544-546 for vhCDR1-3 and SEQ ID NOs: 548-550 for v1CDR1-3 of D99136_2C11 [MICA/B]_H2_D99136_2C11 [MICA/B]_L1, (xl) SEQ ID NOs: 552-554 for vhCDR1-3 and SEQ ID NOs: 556-558 for vlCDR1-3 of D99136_2C11 [MICA/B]_H2_D99136_2C11 [MICA/B]_L2, (xli) SEQ ID NOs: 560-562 for vhCDR1-3 and SEQ ID NOs: 564-566 for v1CDR1-3 of D103388_1B11-2 [MICA/B]_H0_D103388_1B11-2 [MICA/B]_L0, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (lvii) SEQ ID NOs: 680-682 for vhCDR1-3 and SEQ ID NOs: 684-686 for vlCDR1-3 of 3F9[MICA/B]_H0_3F9[MICA/B]_L0, (lviii) SEQ ID NOs: 688-690 for vhCDR1-3 and SEQ ID NOs: 692-694 for vlCDR1-3 of 6E1[MICA/B]_H0_6E1[MICA/B]_L0, (lix) SEQ ID NOs: 696-698 for vhCDR1-3 and SEQ ID NOs: 700-702 for vlCDR1-3 of 7C6[MICA/B]_H0_7C6[MICA/B]_L0, (lx) SEQ ID NOs: 704-706 for vhCDR1-3 and SEQ ID NOs: 708-710 for vlCDR1-3 of 13A9 [MICA/B]_H0_13A9 [MICA/B]_L0, and (lxi) SEQ ID NOs: 712-714 for vhCDR1-3 and SEQ ID NOs: 716-718 for vlCDR1-3 of 1D5 [MICA/B]_H0_1D5 [MICA/B]_L0, as depicted in FIGS. 21 and 22.

In a further embodiment and in accordance with any of the above, the anti-MICA/B scFv comprises a variable heavy domain and variable light domain pair selected from the group including: (i) SEQ ID Nos: 239 and 243 for D94837_E11_1 [MICA/B]_H0_D94837_1E11_1 [MICA/B]_L0, (ii) SEQ ID Nos: 247 and 251 for D94837_1E11_1 [MICA/B]_H1_D94837_1E11_1 [MICA/B]_L1, (iii) SEQ ID Nos: 255 and 259 for D94837_1E1_1 [MICA/B]_H1_D94837_E11_1 [MICA/B]_L2, (iv) SEQ ID Nos: 263 and 267 for D94837_1E11_1 [MICA/B]_H2_D94837_1E11_1 [MICA/B]_L1, (v) SEQ ID Nos: 271 and 275 for D94837_E11_1 [MICA/B]_H2_D94837_E11_1 [MICA/B]_L2, (vi) SEQ ID Nos: 279 and 283 for 2E5 [MICA/B]_H0_2E5 [MICA/B]_L0, (vii) SEQ ID Nos: 287 and 291 for 2E5 [MICA/B]_H1_2E5 [MICA/B]_L1, (viii) SEQ ID Nos: 295 and 299 for 2E5 [MICA/B]_H1_2E5 [MICA/B]_L2, (ix) SEQ ID Nos: 303 and 307 for 2E5 [MICA/B]_H2_2E5 [MICA/B]_L1, (x) SEQ ID Nos: 311 and 315 for 2E5 [MICA/B]_H2_2E5 [MICA/B]_L2, (xi) SEQ ID Nos: 319 and 323 for D94852_2E12 [MICA/B]_H0_D94852_2E12 [MICA/B]_L0, (xii) SEQ ID Nos: 327 and 331 for D94852_2E12 [MICA/B]_H1_D94852_2E12 [MICA/B]_L1, (xiii) SEQ ID Nos: 335 and 339 for D94852_2E12 [MICA/B]_H1_D94852_2E12 [MICA/B]_L2, (xiv) SEQ ID Nos: 343 and 347 for D94852_2E12 [MICA/B]_H2_D94852_2E12 [MICA/B]_L1, (xv) SEQ ID Nos: 351 and 355 for D94852_2E12 [MICA/B]_H2_D94852_2E12 [MICA/B]_L2, (xvi) SEQ ID Nos: 359 and 363 for D99136_2F7 [MICA/B]_H0_D99136_2F7 [MICA/B]_L0, (xvii) SEQ ID Nos: 367 and 371 for D99136_2F7 [MICA/B]_H1_D99136_2F7 [MICA/B]_L1, (xviii) SEQ ID Nos: 375 and 379 for D99136_2F7 [MICA/B]_H1_D99136_2F7 [MICA/B]_L2, (xix) SEQ ID Nos: 383 and 387 for D99136_2F7 [MICA/B]_H2_D99136_2F7 [MICA/B]_L1, (xx) SEQ ID Nos: 391 and 395 for D99136_2F7 [MICA/B]_H2_D99136_2F7 [MICA/B]_L2, (xxi) SEQ ID Nos: 399 and 403 for D103388_1C7 [MICA/B]_H0_D103388_1C7 [MICA/B]_L0, (xxii) SEQ ID Nos: 407 and 411 for D103388_1C7 [MICA/B]_H1_D103388_1C7 [MICA/B]_L1, (xxiii) SEQ ID Nos: 415 and 419 for D103388_1C7 [MICA/B]_H1_D103388_1C7 [MICA/B]_L2, (xxiv) SEQ ID Nos: 423 and 427 for D103388_1C7 [MICA/B]_H2_D103388_1C7 [MICA/B]_L1, (xxv) SEQ ID Nos: 431 and 435 for D103388_1C7 [MICA/B]_H2_D103388_1C7 [MICA/B]_L2, (xxvi) SEQ ID Nos: 439 and 443 for D103388_1D7 [MICA/B]_H0_D103388_1D7 [MICA/B]_L0, (xxvii) SEQ ID Nos: 447 and 451 for D103388_1D7 [MICA/B]_H1_D103388_1D7 [MICA/B]_L1, (xxviii) SEQ ID Nos: 455 and 459 for D103388_1D7 [MICA/B]_H1_D103388_1D7 [MICA/B]_L2, (xxix) SEQ ID Nos: 463 and 467 for D103388_1D7 [MICA/B]_H2_D103388_1D7 [MICA/B]_L1, (xxx) SEQ ID Nos: 471 and 475 for D103388_1D7 [MICA/B]_H2_D103388_1D7 [MICA/B]_L2, (xxxi) SEQ ID Nos: 479 and 483 for D105317_1A2 [MICA/B]_H0_D105317_1A2 [MICA/B]_L0, (xxxii) SEQ ID Nos: 487 and 491 for D105317_1A2 [MICA/B]_H1_D105317_1A2 [MICA/B]_L1, (xxxiii) SEQ ID Nos: 495 and 499 for D105317_1A2 [MICA/B]_H1_D105317_1A2 [MICA/B]_L2, (xxxiv) SEQ ID Nos: 503 and 507 for D105317_1A2 [MICA/B]_H2_D105317_1A2 [MICA/B]_L1, (xxxv) SEQ ID Nos: 511 and 515 for D105317_1A2 [MICA/B]_H2_D105317_1A2 [MICA/B]_L2, (xxxvi) SEQ ID Nos: 519 and 523 for D99136_2C11 [MICA/B]_H0_D99136_2C11 [MICA/B]_L0, (xxxvii) SEQ ID Nos: 527 and 531 for D99136_2C11 [MICA/B]_H1_D99136_2C11 [MICA/B]_L1, (xxxviii) SEQ ID Nos: 535 and 539 for D99136_2C11 [MICA/B]_H1_D99136_2C11 [MICA/B]_L2, (xxxix) SEQ ID Nos: 543 and 547 for D99136_2C11 [MICA/B]_H2_D99136_2C11 [MICA/B]_L1, (xl) SEQ ID Nos: 551 and 555 for D99136_2C11 [MICA/B]_H2_D99136_2C11 [MICA/B]_L2, (xli) SEQ ID Nos: 559 and 563 for D103388_1B11-2 [MICA/B]_H0_D103388_1B11-2 [MICA/B]_L0, (xlii) SEQ ID Nos: 567 and 571 for D105317_1B6 [MICA/B]_H0_D105317_1B6 [MICA/B]_L0, (xliii) SEQ ID Nos: 575 and 579 for D105317_1C8 [MICA/B]_H0_D105317_1C8 [MICA/B]_L0, (xliv) SEQ ID Nos: 583 and 587 for D105317_1F6 [MICA/B]_H0_D105317_1F6 [MICA/B]_L0, (xlv) SEQ ID Nos: 591 and 595 for D105317_1F7 [MICA/B]_H0_D105317_1F7 [MICA/B]_L0, (xlvi) SEQ ID Nos: 599 and 603 for D94837_1D3 [MICA/B]_H0_D94837_1D3 [MICA/B]_L0, (xlvii) SEQ ID Nos: 607 and 611 for D94837_1D8 [MICA/B]_H0_D94837_1D8 [MICA/B]_L0, (xlviii) SEQ ID Nos: 615 and 619 for D94837_1D9 [MICA/B]_H0_D94837_1D9 [MICA/B]_L0, (xlix) SEQ ID Nos: 623 and 627 for D94837_1E1 [MICA/B]_H0_D94837_1E1 [MICA/B]_L0, (1) SEQ ID Nos: 631 and 635 for D94852_2D4 [MICA/B]_H0_D94852_2D4 [MICA/B]_L0, (li) SEQ ID Nos: 639 and 643 for D94852_2G8 [MICA/B]_H0_D94852_2G8 [MICA/B]_L0, (lii) SEQ ID Nos: 647 and 651 for D88487_2A8 [MICA/B]_H0_D88487_2A8 [MICA/B]_L0, (liii) SEQ ID Nos: 655 and 659 for D94837_3B12 [MICA/B]_H0_D94837_3B12 [MICA/B]_L0, (liv) SEQ ID Nos: 663 and 667 for D99122_1H7 [MICA/B]_H0_D99122_1H7 [MICA/B]_L0, (lv) SEQ ID Nos: 671 and 675 for D99136_2E8 [MICA/B]_H0_D99136_2E8 [MICA/B]_L0, (lvi) SEQ ID Nos: 647 and 651 for D88487_2A8 [MICA/B]_H0_D88487_2A8 [MICA/B]_L0, (lvii) SEQ ID Nos: 679 and 683 for 3F9[MICA/B]_H0_3F9[MICA/B]_L0, (lviii) SEQ ID Nos: 687 and 691 for 6E1[MICA/B]_H0_6E1[MICA/B]_L0, (lix) SEQ ID Nos: 695 and 699 for 7C6[MICA/B]_H0_7C6[MICA/B]_L0, (lx) SEQ ID Nos: 703 and 707 for 13A9 [MICA/B]_H0_13A9 [MICA/B]_L0, and (lxi) SEQ ID Nos: 711 and 715 for 1D5 [MICA/B]_H0_1D5 [MICA/B]_L0, as depicted in FIGS. 21 and 22.

In a further embodiment and in accordance with any of the above, the first variable light VL1 domain of the anti-MICA/B scFv is covalently attached to the N-terminus of the first Fc domain using a domain linker, or the first variable heavy VH1 domain of the anti-MICA/B scFv is covalently attached to the N-terminus of the first Fc domain using a domain linker.

In a further embodiment and in accordance with any of the above, the scFv linker is a charged scFv linker.

In a further embodiment and in accordance with the above, the scFv linker is a charged scFv linker having the amino acid sequence (GKPGS)₄ (SEQ ID NO: 1).

In a further embodiment and in accordance with any of the above, the first and second Fc domains are each variant Fc domains.

In a further embodiment and in accordance with the above, the first and/or second variant Fc domains comprise one or more FcγRIIIA (CD16a) binding variant substitutions.

In a further embodiment and in accordance with the above, the one or more FcγRIIIA (CD16a) binding variant substitutions are selected from the group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with the above, the first and second variant Fc domains comprise a set of FcγRIIIA (CD16a) binding variant substitutions selected from the group including: (i) S239D/I332E: S239D/I332E, (ii) S239D: S239D, (iii) 1332E: 1332E, (iv) WT: S239D/I332E, (v) WT: S239D, (vi) WT: 1332E, (vii) S239D/I332E: WT, (viii) S239D: WT, (ix) 1332E: WT, (x) S239D/I332E: S239D, (xi) S239D/I332E: 1332E, (xii) S239D: S239D/I332E, (xiii) 1332E: S239D/I332E, (xiv) S239D: 1332E, and (xv) 1332E: S239D, wherein numbering is according to EU numbering.

In a further embodiment and in accordance with any of the above, the first and/or second variant Fc domains comprise the FcγRIIIA (CD16a) binding variant substitutions of S239D/I332E, wherein numbering is according to EU numbering.

In a further embodiment and in accordance with any of the above, the first and second variant Fc domains comprise a set of heterodimerization variants selected from the group including those depicted in FIGS. 4A-4F, wherein numbering is according to EU numbering.

In a further embodiment and in accordance with the above, the set of heterodimerization variants is selected from the group including: (i) S364K/E357Q: L368D/K370S, (ii) S364K: L368D/K370S, (iii) S364K: L368E/K370S, (iv) D401K: T411E/K360E/Q362E, and (v) T366W: T366S/L368A/Y407V, wherein numbering is according to EU numbering.

In a further embodiment and in accordance with any of the above, the first and second variant Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant Fc domain comprises one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first monomer comprises amino acid variants S364K/E357Q/E233P/L234V/L235A/G236del/S267K, wherein the second monomer comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second monomers each further comprise amino acid variants M428L/N434S, M428L/434A, or M252Y/S254T/T256E, wherein numbering is according to EU numbering.

In another aspect, a nucleic acid composition comprising nucleic acids encoding the first and second monomers and the light chain of the antibody (as described above) is provided.

In another aspect, an expression vector comprising the nucleic acids (as described above) is provided.

In another aspect, a host cell transformed with the expression vector (as described above) is provided.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, the present disclosure provides a heterodimeric antibody, comprising: (a) a first monomer comprising, from N-terminus to C-terminus, a VH1-CH1-[first optional domain linker]-scFv-[second optional domain linker]-CH2-CH3, wherein VH1 is a first variable heavy VH1 domain, scFv is an anti-MICA/B scFv, and CH2-CH3 is a first Fc domain; (b) a second monomer comprising, from N-terminus to C-terminus, a CH2-CH3, wherein the CH2-CH3 is a second Fc domain; and (c) a light chain comprising, from N-terminus to C-terminus, a VL1-CL, wherein VL1 is a first variable light VH1 domain and CL is a constant light domain, wherein: (i) the first variable heavy VH1 domain and the first variable light VL1 domain form a NKp46 antigen binding domain, (ii) the anti-MICA/B scFv comprises a second variable heavy VH2 domain, a scFv linker, and a second variable light VL2 domain, and (iii) the anti-MICA/B scFv is covalently attached between the C-terminus of the CH1 domain and the N-terminus of the first Fc domain.

In a further embodiment and in accordance with the above, the NKp46 antigen binding domain comprises a set of vhCDR1-3 and vlCDR1-3 from a variable heavy domain and variable light domain pair selected from the group including: (i) SEQ ID NOs: 720-722 for vhCDR1-3 and SEQ ID NOs: 724-726 for vlCDR1-3 of 2C10A3.372[NKp46]_H1_2C10A3.372[NKp46]_L1, and (ii) SEQ ID NOs: 728-730 for vhCDR1-3 and SEQ ID NOs: 732-734 for vlCDR1-3 of NKp46-A[NKp46]_H_NKp46-A[NKp46]_L, as depicted in FIGS. 23 and 24.

In a further embodiment and in accordance with any of the above, the NKp46 antigen binding domain comprises a variable heavy domain and variable light domain pair selected from the group including: (i) SEQ ID Nos: 719 and 723 for 2C10A3.372[NKp46]_H1_2C10A3.372[NKp46]_L1, and (ii) SEQ ID Nos: 727 and 731 for NKp46-A[NKp46]_H_NKp46-A[NKp46]_L, as depicted in FIGS. 23 and 24.

In a further embodiment and in accordance with any of the above, the anti-MICA/B scFv comprises a set of vhCDR1-3 and vlCDR1-3 from a variable heavy domain and variable light domain pair selected from the group including: (i) SEQ ID NOs: 240-242 for vhCDR1-3 and SEQ ID NOs: 244-246 for vlCDR1-3 of D94837_E11_1 [MICA/B]_H0_D94837_1E11_1 [MICA/B]_L0, (ii) SEQ ID NOs: 248-250 for vhCDR1-3 and SEQ ID NOs: 252-254 for vlCDR1-3 of D94837_E11_1 [MICA/B]_H1_D94837_1E11_1 [MICA/B]_L1, (iii) SEQ ID NOs: 256-258 for vhCDR1-3 and SEQ ID NOs: 260-262 for vlCDR1-3 of D94837_1E11_1 [MICA/B]_H1_D94837_E11_1 [MICA/B]_L2, (iv) SEQ ID NOs: 264-266 for vhCDR1-3 and SEQ ID NOs: 268-270 for vlCDR1-3 of D94837_E11_1 [MICA/B]_H2_D94837_1E11_1 [MICA/B]_L1, (v) SEQ ID NOs: 272-274 for vhCDR1-3 and SEQ ID NOs: 276-278 for vlCDR1-3 of D94837_E11_1 [MICA/B]_H2_D94837_1E11_1 [MICA/B]_L2, (vi) SEQ ID NOs: 280-282 for vhCDR1-3 and SEQ ID NOs: 284-286 for vlCDR1-3 of 2E5 [MICA/B]_H0_2E5 [MICA/B]_L0, (vii) SEQ ID NOs: 288-290 for vhCDR1-3 and SEQ ID NOs: 292-294 for vlCDR1-3 of 2E5 [MICA/B]_H1_2E5 [MICA/B]_L1, (viii) SEQ ID NOs: 296-298 for vhCDR1-3 and SEQ ID NOs: 300-302 for vlCDR1-3 of 2E5 [MICA/B]_H1_2E5 [MICA/B]_L2, (ix) SEQ ID NOs: 304-306 for vhCDR1-3 and SEQ ID NOs: 308-310 for vlCDR1-3 of 2E5 [MICA/B]_H2_2E5 [MICA/B]_L1, (x) SEQ ID NOs: 312-314 for vhCDR1-3 and SEQ ID NOs: 316-318 for vlCDR1-3 of 2E5 [MICA/B]_H2_2E5 [MICA/B]_L2, (xi) SEQ ID NOs: 320-322 for vhCDR1-3 and SEQ ID NOs: 324-326 for v1CDR1-3 of D94852_2E12 [MICA/B]_H0_D94852_2E12 [MICA/B]_L0, (xii) SEQ ID NOs: 328-330 for vhCDR1-3 and SEQ ID NOs: 332-334 for v1CDR1-3 of D94852_2E12 [MICA/B]_H1_D94852_2E12 [MICA/B]_L1, (xiii) SEQ ID NOs: 336-338 for vhCDR1-3 and SEQ ID NOs: 340-342 for v1CDR1-3 of D94852_2E12 [MICA/B]_H1_D94852_2E12 [MICA/B]_L2, (xiv) SEQ ID NOs: 344-346 for vhCDR1-3 and SEQ ID NOs: 348-350 for v1CDR1-3 of D94852_2E12 [MICA/B]_H2_D94852_2E12 [MICA/B]_L1, (xv) SEQ ID NOs: 352-354 for vhCDR1-3 and SEQ ID NOs: 356-358 for v1CDR1-3 of D94852_2E12 [MICA/B]_H2_D94852_2E12 [MICA/B]_L2, (xvi) SEQ ID NOs: 360-362 for vhCDR1-3 and SEQ ID NOs: 364-366 for v1CDR1-3 of D99136_2F7 [MICA/B]_H0_D99136_2F7 [MICA/B]_L0, (xvii) SEQ ID NOs: 368-370 for vhCDR1-3 and SEQ ID NOs: 372-374 for v1CDR1-3 of D99136_2F7 [MICA/B]_H1_D99136_2F7 [MICA/B]_L1, (xviii) SEQ ID NOs: 376-378 for vhCDR1-3 and SEQ ID NOs: 380-382 for v1CDR1-3 of D99136_2F7 [MICA/B]_H1_D99136_2F7 [MICA/B]_L2, (xix) SEQ ID NOs: 384-386 for vhCDR1-3 and SEQ ID NOs: 388-390 for v1CDR1-3 of D99136_2F7 [MICA/B]_H2_D99136_2F7 [MICA/B]_L1, (xx) SEQ ID NOs: 392-394 for vhCDR1-3 and SEQ ID NOs: 396-398 for v1CDR1-3 of D99136_2F7 [MICA/B]_H2_D99136_2F7 [MICA/B]_L2, (xxi) SEQ ID NOs: 400-402 for vhCDR1-3 and SEQ ID NOs: 404-406 for v1CDR1-3 of D103388_1C7 [MICA/B]_H0_D103388_1C7 [MICA/B]_L0, (xxii) SEQ ID NOs: 408-410 for vhCDR1-3 and SEQ ID NOs: 412-414 for v1CDR1-3 of D103388_1C7 [MICA/B]_H1_D103388_1C7 [MICA/B]_L1, (xxiii) SEQ ID NOs: 416-418 for vhCDR1-3 and SEQ ID NOs: 420-422 for v1CDR1-3 of D103388_1C7 [MICA/B]_H1_D103388_1C7 [MICA/B]_L2, (xxiv) SEQ ID NOs: 424-426 for vhCDR1-3 and SEQ ID NOs: 428-430 for v1CDR1-3 of D103388_1C7 [MICA/B]_H2_D103388_1C7 [MICA/B]_L1, (xxv) SEQ ID NOs: 432-434 for vhCDR1-3 and SEQ ID NOs: 436-438 for v1CDR1-3 of D103388_1C7 [MICA/B]_H2_D103388_1C7 [MICA/B]_L2, (xxvi) SEQ ID NOs: 440-442 for vhCDR1-3 and SEQ ID NOs: 444-446 for v1CDR1-3 of D103388_1D7 [MICA/B]_H0_D103388_1D7 [MICA/B]_L0, (xxvii) SEQ ID NOs: 448-450 for vhCDR1-3 and SEQ ID NOs: 452-454 for v1CDR1-3 of D103388_1D7 [MICA/B]_H1_D103388_1D7 [MICA/B]_L1, (xxviii) SEQ ID NOs: 456-458 for vhCDR1-3 and SEQ ID NOs: 460-462 for v1CDR1-3 of D103388_1D7 [MICA/B]_H1_D103388_1D7 [MICA/B]_L2, (xxix) SEQ ID NOs: 464-466 for vhCDR1-3 and SEQ ID NOs: 468-470 for v1CDR1-3 of D103388_1D7 [MICA/B]_H2_D103388_1D7 [MICA/B]_L1, (xxx) SEQ ID NOs: 472-474 for vhCDR1-3 and SEQ ID NOs: 476-478 for v1CDR1-3 of D103388_1D7 [MICA/B]_H2_D103388_1D7 [MICA/B]_L2, (xxxi) SEQ ID NOs: 480-482 for vhCDR1-3 and SEQ ID NOs: 484-486 for v1CDR1-3 of D105317_1A2 [MICA/B]_H0_D105317_1A2 [MICA/B]_L0, (xxxii) SEQ ID NOs: 488-490 for vhCDR1-3 and SEQ ID NOs: 492-494 for v1CDR1-3 of D105317_1A2 [MICA/B]_H1_D105317_1A2 [MICA/B]_L1, (xxxiii) SEQ ID NOs: 496-498 for vhCDR1-3 and SEQ ID NOs: 500-502 for v1CDR1-3 of D105317_1A2 [MICA/B]_H1_D105317_1A2 [MICA/B]_L2, (xxxiv) SEQ ID NOs: 504-506 for vhCDR1-3 and SEQ ID NOs: 508-510 for v1CDR1-3 of D105317_1A2 [MICA/B]_H2_D105317_1A2 [MICA/B]_L1, (xxxv) SEQ ID NOs: 512-514 for vhCDR1-3 and SEQ ID NOs: 516-518 for vlCDR1-3 of D105317_1A2 [MICA/B]_H2_D105317_1A2 [MICA/B]_L2, (xxxvi) SEQ ID NOs: 520-522 for vhCDR1-3 and SEQ ID NOs: 524-526 for vlCDR1-3 of D99136_2C11 [MICA/B]_H0_D99136_2C11 [MICA/B]_L0, (xxxvii) SEQ ID NOs: 528-530 for vhCDR1-3 and SEQ ID NOs: 532-534 for v1CDR1-3 of D99136_2C11 [MICA/B]_H1_D99136_2C11 [MICA/B]_L1, (xxxviii) SEQ ID NOs: 536-538 for vhCDR1-3 and SEQ ID NOs: 540-542 for v1CDR1-3 of D99136_2C11 [MICA/B]_H1_D99136_2C11 [MICA/B]_L2, (xxxix) SEQ ID NOs: 544-546 for vhCDR1-3 and SEQ ID NOs: 548-550 for v1CDR1-3 of D99136_2C11 [MICA/B]_H2_D99136_2C11 [MICA/B]_L1, (xl) SEQ ID NOs: 552-554 for vhCDR1-3 and SEQ ID NOs: 556-558 for v1CDR1-3 of D99136_2C11 [MICA/B]_H2_D99136_2C11 [MICA/B]_L2, (xli) SEQ ID NOs: 560-562 for vhCDR1-3 and SEQ ID NOs: 564-566 for v1CDR1-3 of D103388_1B11-2 [MICA/B]_H0_D103388_1B11-2 [MICA/B]_L0, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (lvii) SEQ ID NOs: 680-682 for vhCDR1-3 and SEQ ID NOs: 684-686 for vlCDR1-3 of 3F9[MICA/B]_H0_3F9[MICA/B]_L0, (lviii) SEQ ID NOs: 688-690 for vhCDR1-3 and SEQ ID NOs: 692-694 for vlCDR1-3 of 6E1[MICA/B]_H0_6E1[MICA/B]_L0, (lix) SEQ ID NOs: 696-698 for vhCDR1-3 and SEQ ID NOs: 700-702 for vlCDR1-3 of 7C6[MICA/B]_H0_7C6[MICA/B]_L0, (lx) SEQ ID NOs: 704-706 for vhCDR1-3 and SEQ ID NOs: 708-710 for vlCDR1-3 of 13A9 [MICA/B]_H0_13A9 [MICA/B]_L0, and (lxi) SEQ ID NOs: 712-714 for vhCDR1-3 and SEQ ID NOs: 716-718 for vlCDR1-3 of 1D5 [MICA/B]_H0_1D5 [MICA/B]_L0, as depicted in FIGS. 21 and 22.

In a further embodiment and in accordance with any of the above, the anti-MICA/B scFv comprises a variable heavy domain and variable light domain pair selected from the group including: (i) SEQ ID Nos: 239 and 243 for D94837_1E11_1 [MICA/B]_H0_D94837_1E11_1 [MICA/B]_L0, (ii) SEQ ID Nos: 247 and 251 for D94837_1E11_1 [MICA/B]_H1_D94837_1E11_1 [MICA/B]_L1, (iii) SEQ ID Nos: 255 and 259 for D94837_1E11_1 [MICA/B]_H1_D94837_1E1_1 [MICA/B]_L2, (iv) SEQ ID Nos: 263 and 267 for D94837_1E11_1 [MICA/B]_H2_D94837_1E11_1 [MICA/B]_L1, (v) SEQ ID Nos: 271 and 275 for D94837_1E11_1 [MICA/B]_H2_D94837_1E11_1 [MICA/B]_L2, (vi) SEQ ID Nos: 279 and 283 for 2E5 [MICA/B]_H0_2E5 [MICA/B]_L0, (vii) SEQ ID Nos: 287 and 291 for 2E5 [MICA/B]_H1_2E5 [MICA/B]_L1, (viii) SEQ ID Nos: 295 and 299 for 2E5 [MICA/B]_H1_2E5 [MICA/B]_L2, (ix) SEQ ID Nos: 303 and 307 for 2E5 [MICA/B]_H2_2E5 [MICA/B]_L1, (x) SEQ ID Nos: 311 and 315 for 2E5 [MICA/B]_H2_2E5 [MICA/B]_L2, (xi) SEQ ID Nos: 319 and 323 for D94852_2E12 [MICA/B]_H0_D94852_2E12 [MICA/B]_L0, (xii) SEQ ID Nos: 327 and 331 for D94852_2E12 [MICA/B]_H1_D94852_2E12 [MICA/B]_L1, (xiii) SEQ ID Nos: 335 and 339 for D94852_2E12 [MICA/B]_H1_D94852_2E12 [MICA/B]_L2, (xiv) SEQ ID Nos: 343 and 347 for D94852_2E12 [MICA/B]_H2_D94852_2E12 [MICA/B]_L1, (xv) SEQ ID Nos: 351 and 355 for D94852_2E12 [MICA/B]_H2_D94852_2E12 [MICA/B]_L2, (xvi) SEQ ID Nos: 359 and 363 for D99136_2F7 [MICA/B]_H0_D99136_2F7 [MICA/B]_L0, (xvii) SEQ ID Nos: 367 and 371 for D99136_2F7 [MICA/B]_H1_D99136_2F7 [MICA/B]_L1, (xviii) SEQ ID Nos: 375 and 379 for D99136_2F7 [MICA/B]_H1_D99136_2F7 [MICA/B]_L2, (xix) SEQ ID Nos: 383 and 387 for D99136_2F7 [MICA/B]_H2_D99136_2F7 [MICA/B]_L1, (xx) SEQ ID Nos: 391 and 395 for D99136_2F7 [MICA/B]_H2_D99136_2F7 [MICA/B]_L2, (xxi) SEQ ID Nos: 399 and 403 for D103388_1C7 [MICA/B]_H0_D103388_1C7 [MICA/B]_L0, (xxii) SEQ ID Nos: 407 and 411 for D103388_1C7 [MICA/B]_H1_D103388_1C7 [MICA/B]_L1, (xxiii) SEQ ID Nos: 415 and 419 for D103388_1C7 [MICA/B]_H1_D103388_1C7 [MICA/B]_L2, (xxiv) SEQ ID Nos: 423 and 427 for D103388_1C7 [MICA/B]_H2_D103388_1C7 [MICA/B]_L1, (xxv) SEQ ID Nos: 431 and 435 for D103388_1C7 [MICA/B]_H2_D103388_1C7 [MICA/B]_L2, (xxvi) SEQ ID Nos: 439 and 443 for D103388_1D7 [MICA/B]_H0_D103388_1D7 [MICA/B]_L0, (xxvii) SEQ ID Nos: 447 and 451 for D103388_1D7 [MICA/B]_H1_D103388_1D7 [MICA/B]_L1, (xxviii) SEQ ID Nos: 455 and 459 for D103388_1D7 [MICA/B]_H1_D103388_1D7 [MICA/B]_L2, (xxix) SEQ ID Nos: 463 and 467 for D103388_1D7 [MICA/B]_H2_D103388_1D7 [MICA/B]_L1, (xxx) SEQ ID Nos: 471 and 475 for D103388_1D7 [MICA/B]_H2_D103388_1D7 [MICA/B]_L2, (xxxi) SEQ ID Nos: 479 and 483 for D105317_1A2 [MICA/B]_H0_D105317_1A2 [MICA/B]_L0, (xxxii) SEQ ID Nos: 487 and 491 for D105317_1A2 [MICA/B]_H1_D105317_1A2 [MICA/B]_L1, (xxxiii) SEQ ID Nos: 495 and 499 for D105317_1A2 [MICA/B]_H1_D105317_1A2 [MICA/B]_L2, (xxxiv) SEQ ID Nos: 503 and 507 for D105317_1A2 [MICA/B]_H2_D105317_1A2 [MICA/B]_L1, (xxxv) SEQ ID Nos: 511 and 515 for D105317_1A2 [MICA/B]_H2_D105317_1A2 [MICA/B]_L2, (xxxvi) SEQ ID Nos: 519 and 523 for D99136_2C11 [MICA/B]_H0_D99136_2C11 [MICA/B]_L0, (xxxvii) SEQ ID Nos: 527 and 531 for D99136_2C11 [MICA/B]_H1_D99136_2C11 [MICA/B]_L1, (xxxviii) SEQ ID Nos: 535 and 539 for D99136_2C11 [MICA/B]_H1_D99136_2C11 [MICA/B]_L2, (xxxix) SEQ ID Nos: 543 and 547 for D99136_2C11 [MICA/B]_H2_D99136_2C11 [MICA/B]_L1, (xl) SEQ ID Nos: 551 and 555 for D99136_2C11 [MICA/B]_H2_D99136_2C11 [MICA/B]_L2, (xli) SEQ ID Nos: 559 and 563 for D103388_1B11-2 [MICA/B]_H0_D103388_1B11-2 [MICA/B]_L0, (xlii) SEQ ID Nos: 567 and 571 for D105317_1B6 [MICA/B]_H0_D105317_1B6 [MICA/B]_L0, (xliii) SEQ ID Nos: 575 and 579 for D105317_1C8 [MICA/B]_H0_D105317_1C8 [MICA/B]_L0, (xliv) SEQ ID Nos: 583 and 587 for D105317_1F6 [MICA/B]_H0_D105317_1F6 [MICA/B]_L0, (xlv) SEQ ID Nos: 591 and 595 for D105317_1F7 [MICA/B]_H0_D105317_1F7 [MICA/B]_L0, (xlvi) SEQ ID Nos: 599 and 603 for D94837_1D3 [MICA/B]_H0_D94837_1D3 [MICA/B]_L0, (xlvii) SEQ ID Nos: 607 and 611 for D94837_1D8 [MICA/B]_H0_D94837_1D8 [MICA/B]_L0, (xlviii) SEQ ID Nos: 615 and 619 for D94837_1D9 [MICA/B]_H0_D94837_1D9 [MICA/B]_L0, (xlix) SEQ ID Nos: 623 and 627 for D94837_1E1 [MICA/B]_H0_D94837_1E1 [MICA/B]_L0, (1) SEQ ID Nos: 631 and 635 for D94852_2D4 [MICA/B]_H0_D94852_2D4 [MICA/B]_L0, (li) SEQ ID Nos: 639 and 643 for D94852_2G8 [MICA/B]_H0_D94852_2G8 [MICA/B]_L0, (lii) SEQ ID Nos: 647 and 651 for D88487_2A8 [MICA/B]_H0_D88487_2A8 [MICA/B]_L0, (liii) SEQ ID Nos: 655 and 659 for D94837_3B12 [MICA/B]_H0_D94837_3B12 [MICA/B]_L0, (liv) SEQ ID Nos: 663 and 667 for D99122_1H7 [MICA/B]_H0_D99122_1H7 [MICA/B]_L0, (lv) SEQ ID Nos: 671 and 675 for D99136_2E8 [MICA/B]_H0_D99136_2E8 [MICA/B]_L0, (lvi) SEQ ID Nos: 647 and 651 for D88487_2A8 [MICA/B]_H0_D88487_2A8 [MICA/B]_L0, (lvii) SEQ ID Nos: 679 and 683 for 3F9[MICA/B]_H0_3F9[MICA/B]_L0, (lviii) SEQ ID Nos: 687 and 691 for 6E1[MICA/B]_H0_6E1[MICA/B]_L0, (lix) SEQ ID Nos: 695 and 699 for 7C6[MICA/B]_H0_7C6[MICA/B]_L0, (lx) SEQ ID Nos: 703 and 707 for 13A9 [MICA/B]_H0_13A9 [MICA/B]_L0, and (lxi) SEQ ID Nos: 711 and 715 for 1D5 [MICA/B]_H0_1D5 [MICA/B]_L0, as depicted in FIGS. 21 and 22.

In a further embodiment and in accordance with any of the above, wherein: (i) the anti-MICA/B scFv comprises, from N-terminus to C-terminus, VH2-scFv linker-VL2, (ii) the variable heavy domain is covalently attached to the C-terminus of the CH1 domain, optionally using a first domain linker, and (iii) the variable light domain is covalently attached to the N-terminus of the first Fc domain, optionally using a second domain linker; or (i) the anti-MICA/B scFv comprises, from N-terminus to C-terminus, VL2-scFv linker-VH2, (ii) the variable light domain is covalently attached to the C-terminus of the CH1 domain, optionally using a first domain linker, and (iii) the variable heavy domain is covalently attached to the N-terminus of the first Fc domain, optionally using a second domain linker.

In a further embodiment and in accordance with any of the above, the heterodimeric antibody comprises a first amino acid sequence, a second amino acid sequence, and a third amino acid sequence, wherein the first, second, and third amino acid sequences are selected from the group including: (i) the amino acid sequences of SEQ ID NOs: 803-805 of XENP47274, and (ii) the amino acid sequences of SEQ ID NOs: 806-808 of XENP47281, as depicted in FIG. 32.

In a further embodiment and in accordance with any of the above, the scFv linker is a charged scFv linker.

In a further embodiment and in accordance with the above, the scFv linker is a charged scFv linker having the amino acid sequence (GKPGS)₄ (SEQ ID NO: 1).

In a further embodiment and in accordance with any of the above, the first and second Fc domains are each variant Fc domains.

In a further embodiment and in accordance with the above, the first and/or second variant Fc domains comprise one or more FcγRIIIA (CD16a) binding variant substitutions.

In a further embodiment and in accordance with the above, the one or more FcγRIIIA (CD16a) binding variant substitutions are selected from the group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with the above, the first and second variant Fc domains comprise a set of FcγRIIIA (CD16a) binding variant substitutions selected from the group including: (i) S239D/I332E: S239D/I332E, (ii) S239D: S239D, (iii) 1332E: 1332E, (iv) WT: S239D/I332E, (v) WT: S239D, (vi) WT: 1332E, (vii) S239D/I332E: WT, (viii) S239D: WT, (ix) 1332E: WT, (x) S239D/I332E: S239D, (xi) S239D/I332E: 1332E, (xii) S239D: S239D/I332E, (xiii) 1332E: S239D/I332E, (xiv) S239D: 1332E, and (xv) 1332E: S239D, wherein numbering is according to EU numbering.

In a further embodiment and in accordance with any of the above, the first and/or second variant Fc domains comprise the FcγRIIIA (CD16a) binding variant substitutions of S239D/I332E, wherein numbering is according to EU numbering.

In a further embodiment and in accordance with any of the above, the first and second variant Fc domains comprise a set of heterodimerization variants selected from the group including those depicted in FIGS. 4A-4F, wherein numbering is according to EU numbering.

In a further embodiment and in accordance with the above, the set of heterodimerization variants is selected from the group including: (i) S364K/E357Q: L368D/K370S, (ii) S364K: L368D/K370S, (iii) S364K: L368E/K370S, (iv) D401K: T411E/K360E/Q362E, and (v) T366W: T366S/L368A/Y407V, wherein numbering is according to EU numbering.

In a further embodiment and in accordance with any of the above, the first and second variant Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant Fc domain comprises one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first monomer comprises amino acid variants S364K/E357Q/E233P/L234V/L235A/G236del/S267K, wherein the second monomer comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second monomers each further comprise amino acid variants M428L/N434S, M428L/434A, or M252Y/S254T/T256E, wherein numbering is according to EU numbering.

In another aspect, a nucleic acid composition comprising nucleic acids encoding the first and second monomers and the light chain of the antibody (as described above) is provided.

In another aspect, an expression vector comprising the nucleic acids (as described above) is provided.

In another aspect, a host cell transformed with the expression vector (as described above) is provided.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, the present disclosure provides a heterodimeric antibody, comprising: (a) a first monomer comprising, from N-terminus to C-terminus, a VH1-CH1-[first optional domain linker]-scFv-[second optional domain linker]-CH2-CH3, wherein VH1 is a first variable heavy VH1 domain, scFv is an anti-NKp46 scFv, and CH2-CH3 is a first Fc domain; (b) a second monomer comprising, from N-terminus to C-terminus, a CH2-CH3, wherein the CH2-CH3 is a second Fc domain; and (c) a light chain comprising, from N-terminus to C-terminus, a VL1-CL, wherein VL1 is a first variable light VH1 domain and CL is a constant light domain, wherein: (i) the first variable heavy VH1 domain and the first variable light VL1 domain form a MICA/B antigen binding domain, (ii) the anti-NKp46 scFv comprises a second variable heavy VH2 domain, a scFv linker, and a second variable light VL2 domain, and (iii) the anti-NKp46 scFv is covalently attached between the C-terminus of the CH1 domain and the N-terminus of the first Fc domain.

In a further embodiment and in accordance with the above, the MICA/B antigen binding domain comprises a set of vhCDR1-3 and vlCDR1-3 from a variable heavy domain and variable light domain pair selected from the group including: (i) SEQ ID NOs: 240-242 for vhCDR1-3 and SEQ ID NOs: 244-246 for vlCDR1-3 of D94837_1E1_1 [MICA/B]_H0_D94837_1E11_1 [MICA/B]_L0, (ii) SEQ ID NOs: 248-250 for vhCDR1-3 and SEQ ID NOs: 252-254 for v1CDR1-3 of D94837_E11_1 [MICA/B]_H1_D94837_1E11_1 [MICA/B]_L1, (iii) SEQ ID NOs: 256-258 for vhCDR1-3 and SEQ ID NOs: 260-262 for v1CDR1-3 of D94837_1E11_1 [MICA/B]_H1_D94837_E11_1 [MICA/B]_L2, (iv) SEQ ID NOs: 264-266 for vhCDR1-3 and SEQ ID NOs: 268-270 for v1CDR1-3 of D94837_1E11_1 [MICA/B]_H2_D94837_1E11_1 [MICA/B]_L1, (v) SEQ ID NOs: 272-274 for vhCDR1-3 and SEQ ID NOs: 276-278 for v1CDR1-3 of D94837_E11_1 [MICA/B]_H2_D94837_1E11_1 [MICA/B]_L2, (vi) SEQ ID NOs: 280-282 for vhCDR1-3 and SEQ ID NOs: 284-286 for v1CDR1-3 of 2E5 [MICA/B]_H0_2E5 [MICA/B]_L0, (vii) SEQ ID NOs: 288-290 for vhCDR1-3 and SEQ ID NOs: 292-294 for v1CDR1-3 of 2E5 [MICA/B]_H1_2E5 [MICA/B]_L1, (viii) SEQ ID NOs: 296-298 for vhCDR1-3 and SEQ ID NOs: 300-302 for v1CDR1-3 of 2E5 [MICA/B]_H1_2E5 [MICA/B]_L2, (ix) SEQ ID NOs: 304-306 for vhCDR1-3 and SEQ ID NOs: 308-310 for v1CDR1-3 of 2E5 [MICA/B]_H2_2E5 [MICA/B]_L1, (x) SEQ ID NOs: 312-314 for vhCDR1-3 and SEQ ID NOs: 316-318 for v1CDR1-3 of 2E5 [MICA/B]_H2_2E5 [MICA/B]_L2, (xi) SEQ ID NOs: 320-322 for vhCDR1-3 and SEQ ID NOs: 324-326 for v1CDR1-3 of D94852_2E12 [MICA/B]_H0_D94852_2E12 [MICA/B]_L0, (xii) SEQ ID NOs: 328-330 for vhCDR1-3 and SEQ ID NOs: 332-334 for v1CDR1-3 of D94852_2E12 [MICA/B]_H1_D94852_2E12 [MICA/B]_L1, (xiii) SEQ ID NOs: 336-338 for vhCDR1-3 and SEQ ID NOs: 340-342 for v1CDR1-3 of D94852_2E12 [MICA/B]_H1_D94852_2E12 [MICA/B]_L2, (xiv) SEQ ID NOs: 344-346 for vhCDR1-3 and SEQ ID NOs: 348-350 for v1CDR1-3 of D94852_2E12 [MICA/B]_H2_D94852_2E12 [MICA/B]_L1, (xv) SEQ ID NOs: 352-354 for vhCDR1-3 and SEQ ID NOs: 356-358 for v1CDR1-3 of D94852_2E12 [MICA/B]_H2_D94852_2E12 [MICA/B]_L2, (xvi) SEQ ID NOs: 360-362 for vhCDR1-3 and SEQ ID NOs: 364-366 for v1CDR1-3 of D99136_2F7 [MICA/B]_H0_D99136_2F7 [MICA/B]_L0, (xvii) SEQ ID NOs: 368-370 for vhCDR1-3 and SEQ ID NOs: 372-374 for v1CDR1-3 of D99136_2F7 [MICA/B]_H1_D99136_2F7 [MICA/B]_L1, (xviii) SEQ ID NOs: 376-378 for vhCDR1-3 and SEQ ID NOs: 380-382 for v1CDR1-3 of D99136_2F7 [MICA/B]_H1_D99136_2F7 [MICA/B]_L2, (xix) SEQ ID NOs: 384-386 for vhCDR1-3 and SEQ ID NOs: 388-390 for v1CDR1-3 of D99136_2F7 [MICA/B]_H2_D99136_2F7 [MICA/B]_L1, (xx) SEQ ID NOs: 392-394 for vhCDR1-3 and SEQ ID NOs: 396-398 for v1CDR1-3 of D99136_2F7 [MICA/B]_H2_D99136_2F7 [MICA/B]_L2, (xxi) SEQ ID NOs: 400-402 for vhCDR1-3 and SEQ ID NOs: 404-406 for v1CDR1-3 of D103388_1C7 [MICA/B]_H0_D103388_1C7 [MICA/B]_L0, (xxii) SEQ ID NOs: 408-410 for vhCDR1-3 and SEQ ID NOs: 412-414 for v1CDR1-3 of D103388_1C7 [MICA/B]_H1_D103388_1C7 [MICA/B]_L1, (xxiii) SEQ ID NOs: 416-418 for vhCDR1-3 and SEQ ID NOs: 420-422 for v1CDR1-3 of D103388_1C7 [MICA/B]_H1_D103388_1C7 [MICA/B]_L2, (xxiv) SEQ ID NOs: 424-426 for vhCDR1-3 and SEQ ID NOs: 428-430 for v1CDR1-3 of D103388_1C7 [MICA/B]_H2_D103388_1C7 [MICA/B]_L1, (xxv) SEQ ID NOs: 432-434 for vhCDR1-3 and SEQ ID NOs: 436-438 for v1CDR1-3 of D103388_1C7 [MICA/B]_H2_D103388_1C7 [MICA/B]_L2, (xxvi) SEQ ID NOs: 440-442 for vhCDR1-3 and SEQ ID NOs: 444-446 for v1CDR1-3 of D103388_1D7 [MICA/B]_H0_D103388_1D7 [MICA/B]_L0, (xxvii) SEQ ID NOs: 448-450 for vhCDR1-3 and SEQ ID NOs: 452-454 for v1CDR1-3 of D103388_1D7 [MICA/B]_H1_D103388_1D7 [MICA/B]_L1, (xxviii) SEQ ID NOs: 456-458 for vhCDR1-3 and SEQ ID NOs: 460-462 for v1CDR1-3 of D103388_1D7 [MICA/B]_H1_D103388_1D7 [MICA/B]_L2, (xxix) SEQ ID NOs: 464-466 for vhCDR1-3 and SEQ ID NOs: 468-470 for v1CDR1-3 of D103388_1D7 [MICA/B]_H2_D103388_1D7 [MICA/B]_L1, (xxx) SEQ ID NOs: 472-474 for vhCDR1-3 and SEQ ID NOs: 476-478 for v1CDR1-3 of D103388_1D7 [MICA/B]_H2_D103388_1D7 [MICA/B]_L2, (xxxi) SEQ ID NOs: 480-482 for vhCDR1-3 and SEQ ID NOs: 484-486 for v1CDR1-3 of D105317_1A2 [MICA/B]_H0_D105317_1A2 [MICA/B]_L0, (xxxii) SEQ ID NOs: 488-490 for vhCDR1-3 and SEQ ID NOs: 492-494 for v1CDR1-3 of D105317_1A2 [MICA/B]_H1_D105317_1A2 [MICA/B]_L1, (xxxiii) SEQ ID NOs: 496-498 for vhCDR1-3 and SEQ ID NOs: 500-502 for v1CDR1-3 of D105317_1A2 [MICA/B]_H1_D105317_1A2 [MICA/B]_L2, (xxxiv) SEQ ID NOs: 504-506 for vhCDR1-3 and SEQ ID NOs: 508-510 for v1CDR1-3 of D105317_1A2 [MICA/B]_H2_D105317_1A2 [MICA/B]_L1, (xxxv) SEQ ID NOs: 512-514 for vhCDR1-3 and SEQ ID NOs: 516-518 for vlCDR1-3 of D105317_1A2 [MICA/B]_H2_D105317_1A2 [MICA/B]_L2, (xxxvi) SEQ ID NOs: 520-522 for vhCDR1-3 and SEQ ID NOs: 524-526 for vlCDR1-3 of D99136_2C11 [MICA/B]_H0_D99136_2C11 [MICA/B]_L0, (xxxvii) SEQ ID NOs: 528-530 for vhCDR1-3 and SEQ ID NOs: 532-534 for v1CDR1-3 of D99136_2C11 [MICA/B]_H1_D99136_2C11 [MICA/B]_L1, (xxxviii) SEQ ID NOs: 536-538 for vhCDR1-3 and SEQ ID NOs: 540-542 for v1CDR1-3 of D99136_2C11 [MICA/B]_H1_D99136_2C11 [MICA/B]_L2, (xxxix) SEQ ID NOs: 544-546 for vhCDR1-3 and SEQ ID NOs: 548-550 for v1CDR1-3 of D99136_2C11 [MICA/B]_H2_D99136_2C11 [MICA/B]_L1, (xl) SEQ ID NOs: 552-554 for vhCDR1-3 and SEQ ID NOs: 556-558 for vlCDR1-3 of D99136_2C11 [MICA/B]_H2_D99136_2C11 [MICA/B]_L2, (xli) SEQ ID NOs: 560-562 for vhCDR1-3 and SEQ ID NOs: 564-566 for v1CDR1-3 of D103388_1B11-2 [MICA/B]_H0_D103388_1B11-2 [MICA/B]_L0, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (lvii) SEQ ID NOs: 680-682 for vhCDR1-3 and SEQ ID NOs: 684-686 for vlCDR1-3 of 3F9[MICA/B]_H0_3F9[MICA/B]_L0, (lviii) SEQ ID NOs: 688-690 for vhCDR1-3 and SEQ ID NOs: 692-694 for vlCDR1-3 of 6E1[MICA/B]_H0_6E1[MICA/B]_L0, (lix) SEQ ID NOs: 696-698 for vhCDR1-3 and SEQ ID NOs: 700-702 for vlCDR1-3 of 7C6[MICA/B]_H0_7C6[MICA/B]_L0, (lx) SEQ ID NOs: 704-706 for vhCDR1-3 and SEQ ID NOs: 708-710 for vlCDR1-3 of 13A9 [MICA/B]_H0_13A9 [MICA/B]_L0, and (lxi) SEQ ID NOs: 712-714 for vhCDR1-3 and SEQ ID NOs: 716-718 for vlCDR1-3 of 1D5 [MICA/B]_H0_1D5 [MICA/B]_L0, as depicted in FIGS. 21 and 22.

In a further embodiment and in accordance with any of the above, the MICA/B antigen binding domain comprises a variable heavy domain and variable light domain pair selected from the group including: (i) SEQ ID Nos: 239 and 243 for D94837_1E11_1 [MICA/B]_H0_D94837_1E11_1 [MICA/B]_L0, (ii) SEQ ID Nos: 247 and 251 for D94837_1E1_1 [MICA/B]_H1_D94837_E11_1 [MICA/B]_L1, (iii) SEQ ID Nos: 255 and 259 for D94837_1E11_1 [MICA/B]_H1_D94837_1E11_1 [MICA/B]_L2, (iv) SEQ ID Nos: 263 and 267 for D94837_E11_1 [MICA/B]_H2_D94837_1E11_1 [MICA/B]_L1, (v) SEQ ID Nos: 271 and 275 for D94837_E11_1 [MICA/B]_H2_D94837_E11_1 [MICA/B]_L2, (vi) SEQ ID Nos: 279 and 283 for 2E5 [MICA/B]_H0_2E5 [MICA/B]_L0, (vii) SEQ ID Nos: 287 and 291 for 2E5 [MICA/B]_H1_2E5 [MICA/B]_L1, (viii) SEQ ID Nos: 295 and 299 for 2E5 [MICA/B]_H1_2E5 [MICA/B]_L2, (ix) SEQ ID Nos: 303 and 307 for 2E5 [MICA/B]_H2_2E5 [MICA/B]_L1, (x) SEQ ID Nos: 311 and 315 for 2E5 [MICA/B]_H2_2E5 [MICA/B]_L2, (xi) SEQ ID Nos: 319 and 323 for D94852_2E12 [MICA/B]_H0_D94852_2E12 [MICA/B]_L0, (xii) SEQ ID Nos: 327 and 331 for D94852_2E12 [MICA/B]_H1_D94852_2E12 [MICA/B]_L1, (xiii) SEQ ID Nos: 335 and 339 for D94852_2E12 [MICA/B]_H1_D94852_2E12 [MICA/B]_L2, (xiv) SEQ ID Nos: 343 and 347 for D94852_2E12 [MICA/B]_H2_D94852_2E12 [MICA/B]_L1, (xv) SEQ ID Nos: 351 and 355 for D94852_2E12 [MICA/B]_H2_D94852_2E12 [MICA/B]_L2, (xvi) SEQ ID Nos: 359 and 363 for D99136_2F7 [MICA/B]_H0_D99136_2F7 [MICA/B]_L0, (xvii) SEQ ID Nos: 367 and 371 for D99136_2F7 [MICA/B]_H1_D99136_2F7 [MICA/B]_L1, (xviii) SEQ ID Nos: 375 and 379 for D99136_2F7 [MICA/B]_H1_D99136_2F7 [MICA/B]_L2, (xix) SEQ ID Nos: 383 and 387 for D99136_2F7 [MICA/B]_H2_D99136_2F7 [MICA/B]_L1, (xx) SEQ ID Nos: 391 and 395 for D99136_2F7 [MICA/B]_H2_D99136_2F7 [MICA/B]_L2, (xxi) SEQ ID Nos: 399 and 403 for D103388_1C7 [MICA/B]_H0_D103388_1C7 [MICA/B]_L0, (xxii) SEQ ID Nos: 407 and 411 for D103388_1C7 [MICA/B]_H1_D103388_1C7 [MICA/B]_L1, (xxiii) SEQ ID Nos: 415 and 419 for D103388_1C7 [MICA/B]_H1_D103388_1C7 [MICA/B]_L2, (xxiv) SEQ ID Nos: 423 and 427 for D103388_1C7 [MICA/B]_H2_D103388_1C7 [MICA/B]_L1, (xxv) SEQ ID Nos: 431 and 435 for D103388_1C7 [MICA/B]_H2_D103388_1C7 [MICA/B]_L2, (xxvi) SEQ ID Nos: 439 and 443 for D103388_1D7 [MICA/B]_H0_D103388_1D7 [MICA/B]_L0, (xxvii) SEQ ID Nos: 447 and 451 for D103388_1D7 [MICA/B]_H1_D103388_1D7 [MICA/B]_L1, (xxviii) SEQ ID Nos: 455 and 459 for D103388_1D7 [MICA/B]_H1D103388_1D7 [MICA/B]_L2, (xxix) SEQ ID Nos: 463 and 467 for D103388_1D7 [MICA/B]_H2_D103388_1D7 [MICA/B]_L1, (xxx) SEQ ID Nos: 471 and 475 for D103388_1D7 [MICA/B]_H2_D103388_1D7 [MICA/B]_L2, (xxxi) SEQ ID Nos: 479 and 483 for D105317_1A2 [MICA/B]_H0_D105317_1A2 [MICA/B]_L0, (xxxii) SEQ ID Nos: 487 and 491 for D105317_1A2 [MICA/B]_H1_D105317_1A2 [MICA/B]_L1, (xxxiii) SEQ ID Nos: 495 and 499 for D105317_1A2 [MICA/B]_H1_D105317_1A2 [MICA/B]_L2, (xxxiv) SEQ ID Nos: 503 and 507 for D105317_1A2 [MICA/B]_H2_D105317_1A2 [MICA/B]_L1, (xxxv) SEQ ID Nos: 511 and 515 for D105317_1A2 [MICA/B]_H2_D105317_1A2 [MICA/B]_L2, (xxxvi) SEQ ID Nos: 519 and 523 for D99136_2C11 [MICA/B]_H0_D99136_2C11 [MICA/B]_L0, (xxxvii) SEQ ID Nos: 527 and 531 for D99136_2C11 [MICA/B]_H1_D99136_2C11 [MICA/B]_L1, (xxxviii) SEQ ID Nos: 535 and 539 for D99136_2C11 [MICA/B]_H1_D99136_2C11 [MICA/B]_L2, (xxxix) SEQ ID Nos: 543 and 547 for D99136_2C11 [MICA/B]_H2_D99136_2C11 [MICA/B]_L1, (xl) SEQ ID Nos: 551 and 555 for D99136_2C11 [MICA/B]_H2_D99136_2C11 [MICA/B]_L2, (xli) SEQ ID Nos: 559 and 563 for D103388_1B11-2 [MICA/B]_H0_D103388_1B11-2 [MICA/B]_L0, (xlii) SEQ ID Nos: 567 and 571 for D105317_1B6 [MICA/B]_H0_D105317_1B6 [MICA/B]_L0, (xliii) SEQ ID Nos: 575 and 579 for D105317_1C8 [MICA/B]_H0_D105317_1C8 [MICA/B]_L0, (xliv) SEQ ID Nos: 583 and 587 for D105317_1F6 [MICA/B]_H0_D105317_1F6 [MICA/B]_L0, (xlv) SEQ ID Nos: 591 and 595 for D105317_1F7 [MICA/B]_H0_D105317_1F7 [MICA/B]_L0, (xlvi) SEQ ID Nos: 599 and 603 for D94837_1D3 [MICA/B]_H0_D94837_1D3 [MICA/B]_L0, (xlvii) SEQ ID Nos: 607 and 611 for D94837_1D8 [MICA/B]_H0_D94837_1D8 [MICA/B]_L0, (xlviii) SEQ ID Nos: 615 and 619 for D94837_1D9 [MICA/B]_H0_D94837_1D9 [MICA/B]_L0, (xlix) SEQ ID Nos: 623 and 627 for D94837_1E1 [MICA/B]_H0_D94837_1E1 [MICA/B]_L0, (1) SEQ ID Nos: 631 and 635 for D94852_2D4 [MICA/B]_H0_D94852_2D4 [MICA/B]_L0, (li) SEQ ID Nos: 639 and 643 for D94852_2G8 [MICA/B]_H0_D94852_2G8 [MICA/B]_L0, (lii) SEQ ID Nos: 647 and 651 for D88487_2A8 [MICA/B]_H0_D88487_2A8 [MICA/B]_L0, (liii) SEQ ID Nos: 655 and 659 for D94837_3B12 [MICA/B]_H0_D94837_3B12 [MICA/B]_L0, (liv) SEQ ID Nos: 663 and 667 for D99122_1H7 [MICA/B]_H0_D99122_1H7 [MICA/B]_L0, (lv) SEQ ID Nos: 671 and 675 for D99136_2E8 [MICA/B]_H0_D99136_2E8 [MICA/B]_L0, (lvi) SEQ ID Nos: 647 and 651 for D88487_2A8 [MICA/B]_H0_D88487_2A8 [MICA/B]_L0, (lvii) SEQ ID Nos: 679 and 683 for 3F9[MICA/B]_H0_3F9[MICA/B]_L0, (lviii) SEQ ID Nos: 687 and 691 for 6E1[MICA/B]_H0_6E1[MICA/B]_L0, (lix) SEQ ID Nos: 695 and 699 for 7C6[MICA/B]_H0_7C6[MICA/B]_L0, (lx) SEQ ID Nos: 703 and 707 for 13A9 [MICA/B]_H0_13A9 [MICA/B]_L0, and (lxi) SEQ ID Nos: 711 and 715 for 1D5 [MICA/B]_H0_1D5 [MICA/B]_L0, as depicted in FIGS. 21 and 22.

In a further embodiment and in accordance with any of the above, the anti-NKp46 scFv comprises a set of vhCDR1-3 and vlCDR1-3 from a variable heavy domain and variable light domain pair selected from the group including: (i) SEQ ID NOs: 720-722 for vhCDR1-3 and SEQ ID NOs: 724-726 for vlCDR1-3 of 2C10A3.372[NKp46]_H1_2C10A3.372[NKp46]_L1, and (ii) SEQ ID NOs: 728-730 for vhCDR1-3 and SEQ ID NOs: 732-734 for vlCDR1-3 of NKp46-A[NKp46]_H_NKp46-A[NKp46]_L, as depicted in FIGS. 23 and 24.

In a further embodiment and in accordance with any of the above, the anti-NKp46 scFv comprises a variable heavy domain and variable light domain pair selected from the group including: (i) SEQ ID Nos: 719 and 723 for 2C10A3.372[NKp46]_H1_2C10A3.372[NKp46]_L1, and (ii) SEQ ID Nos: 727 and 731 for NKp46-A[NKp46]_H_NKp46-A[NKp46]_L, as depicted in FIGS. 23 and 24.

In a further embodiment and in accordance with any of the above, wherein: (i) the anti-NKp46 scFv comprises, from N-terminus to C-terminus, VH2-scFv linker-VL2, (ii) the variable heavy domain is covalently attached to the C-terminus of the CH1 domain, optionally using a first domain linker, and (iii) the variable light domain is covalently attached to the N-terminus of the first Fc domain, optionally using a second domain linker; or (i) the anti-NKp46 scFv comprises, from N-terminus to C-terminus, VL2-scFv linker-VH2, (ii) the variable light domain is covalently attached to the C-terminus of the CH1 domain, optionally using a first domain linker, and (iii) the variable heavy domain is covalently attached to the N-terminus of the first Fc domain, optionally using a second domain linker.

In a further embodiment and in accordance with any of the above, the scFv linker is a charged scFv linker.

In a further embodiment and in accordance with the above, the scFv linker is a charged scFv linker having the amino acid sequence (GKPGS)₄ (SEQ ID NO: 1).

In a further embodiment and in accordance with any of the above, the first and second Fc domains are each variant Fc domains.

In a further embodiment and in accordance with the above, the first and/or second variant Fc domains comprise one or more FcγRIIIA (CD16a) binding variant substitutions.

In a further embodiment and in accordance with the above, the one or more FcγRIIIA (CD16a) binding variant substitutions are selected from the group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with the above, the first and second variant Fc domains comprise a set of FcγRIIIA (CD16a) binding variant substitutions selected from the group including: (i) S239D/I332E: S239D/I332E, (ii) S239D: S239D, (iii) I332E: I332E, (iv) WT: S239D/I332E, (v) WT: S239D, (vi) WT: 1332E, (vii) S239D/I332E: WT, (viii) S239D: WT, (ix) 1332E: WT, (x) S239D/I332E: S239D, (xi) S239D/I332E: 1332E, (xii) S239D: S239D/I332E, (xiii) 1332E: S239D/I332E, (xiv) S239D: 1332E, and (xv) 1332E: S239D, wherein numbering is according to EU numbering.

In a further embodiment and in accordance with any of the above, the first and/or second variant Fc domains comprise the FcγRIIIA (CD16a) binding variant substitutions of S239D/I332E, wherein numbering is according to EU numbering.

In a further embodiment and in accordance with any of the above, the first and second variant Fc domains comprise a set of heterodimerization variants selected from the group including those depicted in FIGS. 4A-4F, wherein numbering is according to EU numbering.

In a further embodiment and in accordance with the above, the set of heterodimerization variants is selected from the group including: (i) S364K/E357Q: L368D/K370S, (ii) S364K: L368D/K370S, (iii) S364K: L368E/K370S, (iv) D401K: T411E/K360E/Q362E, and (v) T366W: T366S/L368A/Y407V, wherein numbering is according to EU numbering.

In a further embodiment and in accordance with any of the above, the first and second variant Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant Fc domain comprises one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first monomer comprises amino acid variants S364K/E357Q/E233P/L234V/L235A/G236del/S267K, wherein the second monomer comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second monomers each further comprise amino acid variants M428L/N434S, M428L/434A, or M252Y/S254T/T256E, wherein numbering is according to EU numbering.

In another aspect, a nucleic acid composition comprising nucleic acids encoding the first and second monomers and the light chain of the antibody (as described above) is provided.

In another aspect, an expression vector comprising the nucleic acids (as described above) is provided.

In another aspect, a host cell transformed with the expression vector (as described above) is provided.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, the present disclosure provides a heterodimeric antibody, comprising: (a) a first monomer comprising, from N-terminus to C-terminus, a VH1-CH1-first linker-scFv-second linker-CH2-CH3, wherein the VH1 is a first variable heavy domain, the scFv is an anti-NKp46 scFv, and the CH2-CH3 is a first Fc domain; (b) a second monomer comprising, from N-terminus to C-terminus, a VH2-CH1-hinge-CH2-CH3, wherein the VH2 is a second variable heavy domain and the CH2-CH3 is a second Fc domain; and (c) a common light chain comprising, from N-terminus to C-terminus, a VL-CL, wherein: (i) the VL is a variable light domain and the CL is a light chain constant domain, (ii) the first variable heavy VH1 domain and the variable light VL domain form a first MICA/B antigen binding domain, and (iii) the second variably heavy VH2 domain and the variable light VL domain form a second MICA/B antigen binding domain.

In a further embodiment and in accordance with the above, the first and/or second MICA/B antigen binding domains comprise a set of vhCDR1-3 and vlCDR1-3 from a variable heavy domain and variable light domain pair selected from the group including: (i) SEQ ID NOs: 240-242 for vhCDR1-3 and SEQ ID NOs: 244-246 for vlCDR1-3 of D94837_1E1_1 [MICA/B]_H0_D94837_E11_1 [MICA/B]_L0, (ii) SEQ ID NOs: 248-250 for vhCDR1-3 and SEQ ID NOs: 252-254 for vlCDR1-3 of D94837_E11_1 [MICA/B]_H1_D94837_1E11_1 [MICA/B]_L1, (iii) SEQ ID NOs: 256-258 for vhCDR1-3 and SEQ ID NOs: 260-262 for vlCDR1-3 of D94837_E11_1 [MICA/B]_H1_D94837_1E11_1 [MICA/B]_L2, (iv) SEQ ID NOs: 264-266 for vhCDR1-3 and SEQ ID NOs: 268-270 for vlCDR1-3 of D94837_1E11_1 [MICA/B]_H2_D94837_1E11_1 [MICA/B]_L1, (v) SEQ ID NOs: 272-274 for vhCDR1-3 and SEQ ID NOs: 276-278 for vlCDR1-3 of D94837_E11_1 [MICA/B]_H2_D94837_1E11_1 [MICA/B]_L2, (vi) SEQ ID NOs: 280-282 for vhCDR1-3 and SEQ ID NOs: 284-286 for vlCDR1-3 of 2E5 [MICA/B]_H0_2E5 [MICA/B]_L0, (vii) SEQ ID NOs: 288-290 for vhCDR1-3 and SEQ ID NOs: 292-294 for vlCDR1-3 of 2E5 [MICA/B]_H1_2E5 [MICA/B]_L1, (viii) SEQ ID NOs: 296-298 for vhCDR1-3 and SEQ ID NOs: 300-302 for vlCDR1-3 of 2E5 [MICA/B]_H1_2E5 [MICA/B]_L2, (ix) SEQ ID NOs: 304-306 for vhCDR1-3 and SEQ ID NOs: 308-310 for v1CDR1-3 of 2E5 [MICA/B]_H2_2E5 [MICA/B]_L1, (x) SEQ ID NOs: 312-314 for vhCDR1-3 and SEQ ID NOs: 316-318 for v1CDR1-3 of 2E5 [MICA/B]_H2_2E5 [MICA/B]_L2, (xi) SEQ ID NOs: 320-322 for vhCDR1-3 and SEQ ID NOs: 324-326 for v1CDR1-3 of D94852_2E12 [MICA/B]_H0_D94852_2E12 [MICA/B]_L0, (xii) SEQ ID NOs: 328-330 for vhCDR1-3 and SEQ ID NOs: 332-334 for v1CDR1-3 of D94852_2E12 [MICA/B]_H1_D94852_2E12 [MICA/B]_L1, (xiii) SEQ ID NOs: 336-338 for vhCDR1-3 and SEQ ID NOs: 340-342 for v1CDR1-3 of D94852_2E12 [MICA/B]_H1_D94852_2E12 [MICA/B]_L2, (xiv) SEQ ID NOs: 344-346 for vhCDR1-3 and SEQ ID NOs: 348-350 for v1CDR1-3 of D94852_2E12 [MICA/B]_H2_D94852_2E12 [MICA/B]_L1, (xv) SEQ ID NOs: 352-354 for vhCDR1-3 and SEQ ID NOs: 356-358 for v1CDR1-3 of D94852_2E12 [MICA/B]_H2_D94852_2E12 [MICA/B]_L2, (xvi) SEQ ID NOs: 360-362 for vhCDR1-3 and SEQ ID NOs: 364-366 for v1CDR1-3 of D99136_2F7 [MICA/B]_H0_D99136_2F7 [MICA/B]_L0, (xvii) SEQ ID NOs: 368-370 for vhCDR1-3 and SEQ ID NOs: 372-374 for v1CDR1-3 of D99136_2F7 [MICA/B]_H1_D99136_2F7 [MICA/B]_L1, (xviii) SEQ ID NOs: 376-378 for vhCDR1-3 and SEQ ID NOs: 380-382 for v1CDR1-3 of D99136_2F7 [MICA/B]_H1_D99136_2F7 [MICA/B]_L2, (xix) SEQ ID NOs: 384-386 for vhCDR1-3 and SEQ ID NOs: 388-390 for v1CDR1-3 of D99136_2F7 [MICA/B]_H2_D99136_2F7 [MICA/B]_L1, (xx) SEQ ID NOs: 392-394 for vhCDR1-3 and SEQ ID NOs: 396-398 for v1CDR1-3 of D99136_2F7 [MICA/B]_H2_D99136_2F7 [MICA/B]_L2, (xxi) SEQ ID NOs: 400-402 for vhCDR1-3 and SEQ ID NOs: 404-406 for v1CDR1-3 of D103388_1C7 [MICA/B]_H0_D103388_1C7 [MICA/B]_L0, (xxii) SEQ ID NOs: 408-410 for vhCDR1-3 and SEQ ID NOs: 412-414 for v1CDR1-3 of D103388_1C7 [MICA/B]_H1_D103388_1C7 [MICA/B]_L1, (xxiii) SEQ ID NOs: 416-418 for vhCDR1-3 and SEQ ID NOs: 420-422 for v1CDR1-3 of D103388_1C7 [MICA/B]_H1_D103388_1C7 [MICA/B]_L2, (xxiv) SEQ ID NOs: 424-426 for vhCDR1-3 and SEQ ID NOs: 428-430 for v1CDR1-3 of D103388_1C7 [MICA/B]_H2_D103388_1C7 [MICA/B]_L1, (xxv) SEQ ID NOs: 432-434 for vhCDR1-3 and SEQ ID NOs: 436-438 for v1CDR1-3 of D103388_1C7 [MICA/B]_H2_D103388_1C7 [MICA/B]_L2, (xxvi) SEQ ID NOs: 440-442 for vhCDR1-3 and SEQ ID NOs: 444-446 for v1CDR1-3 of D103388_1D7 [MICA/B]_H0_D103388_1D7 [MICA/B]_L0, (xxvii) SEQ ID NOs: 448-450 for vhCDR1-3 and SEQ ID NOs: 452-454 for v1CDR1-3 of D103388_1D7 [MICA/B]_H1_D103388_1D7 [MICA/B]_L1, (xxviii) SEQ ID NOs: 456-458 for vhCDR1-3 and SEQ ID NOs: 460-462 for v1CDR1-3 of D103388_1D7 [MICA/B]_H1_D103388_1D7 [MICA/B]_L2, (xxix) SEQ ID NOs: 464-466 for vhCDR1-3 and SEQ ID NOs: 468-470 for v1CDR1-3 of D103388_1D7 [MICA/B]_H2_D103388_1D7 [MICA/B]_L1, (xxx) SEQ ID NOs: 472-474 for vhCDR1-3 and SEQ ID NOs: 476-478 for v1CDR1-3 of D103388_1D7 [MICA/B]_H2_D103388_1D7 [MICA/B]_L2, (xxxi) SEQ ID NOs: 480-482 for vhCDR1-3 and SEQ ID NOs: 484-486 for v1CDR1-3 of D105317_1A2 [MICA/B]_H0_D105317_1A2 [MICA/B]_L0, (xxxii) SEQ ID NOs: 488-490 for vhCDR1-3 and SEQ ID NOs: 492-494 for v1CDR1-3 of D105317_1A2 [MICA/B]_H1_D105317_1A2 [MICA/B]_L1, (xxxiii) SEQ ID NOs: 496-498 for vhCDR1-3 and SEQ ID NOs: 500-502 for v1CDR1-3 of D105317_1A2 [MICA/B]_H1_D105317_1A2 [MICA/B]_L2, (xxxiv) SEQ ID NOs: 504-506 for vhCDR1-3 and SEQ ID NOs: 508-510 for vlCDR1-3 of D105317_1A2 [MICA/B]_H2_D105317_1A2 [MICA/B]_L1, (xxxv) SEQ ID NOs: 512-514 for vhCDR1-3 and SEQ ID NOs: 516-518 for v1CDR1-3 of D105317_1A2 [MICA/B]_H2_D105317_1A2 [MICA/B]_L2, (xxxvi) SEQ ID NOs: 520-522 for vhCDR1-3 and SEQ ID NOs: 524-526 for v1CDR1-3 of D99136_2C11 [MICA/B]_H0_D99136_2C11 [MICA/B]_L0, (xxxvii) SEQ ID NOs: 528-530 for vhCDR1-3 and SEQ ID NOs: 532-534 for vlCDR1-3 of D99136_2C11 [MICA/B]_H1_D99i36_2C11 [MICA/B]_L1, (xxxviii) SEQ ID NOs: 536-538 for vhCDR1-3 and SEQ ID NOs: 540-542 for v1CDR1-3 of D99136_2C11 [MICA/B]_H1_D99136_2C11 [MICA/B]_L2, (xxxix) SEQ ID NOs: 544-546 for vhCDR1-3 and SEQ ID NOs: 548-550 for v1CDR1-3 of D99136_2C11 [MICA/B]_H2_D99136_2C11 [MICA/B]_L1, (xl) SEQ ID NOs: 552-554 for vhCDR1-3 and SEQ ID NOs: 556-558 for v1CDR1-3 of D99136_2C11 [MICA/B]_H2_D99136_2C11 [MICA/B]_L2, (xli) SEQ ID NOs: 560-562 for vhCDR1-3 and SEQ ID NOs: 564-566 for v1CDR1-3 of D103388_1B11-2 [MICA/B]_H0_D103388_1B11-2 [MICA/B]_L0, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (lvii) SEQ ID NOs: 680-682 for vhCDR1-3 and SEQ ID NOs: 684-686 for vlCDR1-3 of 3F9[MICA/B]_H0_3F9[MICA/B]_L0, (lviii) SEQ ID NOs: 688-690 for vhCDR1-3 and SEQ ID NOs: 692-694 for vlCDR1-3 of 6E1[MICA/B]_H0_6E1[MICA/B]_L0, (lix) SEQ ID NOs: 696-698 for vhCDR1-3 and SEQ ID NOs: 700-702 for vlCDR1-3 of 7C6[MICA/B]_H0_7C6[MICA/B]_L0, (lx) SEQ ID NOs: 704-706 for vhCDR1-3 and SEQ ID NOs: 708-710 for vlCDR1-3 of 13A9 [MICA/B]_H0_13A9 [MICA/B]_L0, and (lxi) SEQ ID NOs: 712-714 for vhCDR1-3 and SEQ ID NOs: 716-718 for vlCDR1-3 of 1D5 [MICA/B]_H0_1D5 [MICA/B]_L0, as depicted in FIGS. 21 and 22.

In a further embodiment and in accordance with any of the above, the first and/or second MICA/B antigen binding domains comprise a variable heavy domain and variable light domain pair selected from the group including: (i) SEQ ID Nos: 239 and 243 for D94837_1E11_1 [MICA/B]_H0_D94837_1E11_1 [MICA/B]_L0, (ii) SEQ ID Nos: 247 and 251 for D94837_1E11_1 [MICA/B]_H1_D94837_E11_1 [MICA/B]_L1, (iii) SEQ ID Nos: 255 and 259 for D94837_1E11_1 [MICA/B]_H1_D94837_1E11_1 [MICA/B]_L2, (iv) SEQ ID Nos: 263 and 267 for D94837_E11_1 [MICA/B]_H2_D94837_E11_1 [MICA/B]_L1, (v) SEQ ID Nos: 271 and 275 for D948371E11_1 [MICA/B]_H2_D94837_E11_1 [MICA/B]_L2, (vi) SEQ ID Nos: 279 and 283 for 2E5 [MICA/B]_H0_2E5 [MICA/B]_L0, (vii) SEQ ID Nos: 287 and 291 for 2E5 [MICA/B]_H1_2E5 [MICA/B]_L1, (viii) SEQ ID Nos: 295 and 299 for 2E5 [MICA/B]_H1_2E5 [MICA/B]_L2, (ix) SEQ ID Nos: 303 and 307 for 2E5 [MICA/B]_H2_2E5 [MICA/B]_L1, (x) SEQ ID Nos: 311 and 315 for 2E5 [MICA/B]_H2_2E5 [MICA/B]_L2, (xi) SEQ ID Nos: 319 and 323 for D94852_2E12 [MICA/B]_H0_D94852_2E12 [MICA/B]_L0, (xii) SEQ ID Nos: 327 and 331 for D94852_2E12 [MICA/B]_H1_D94852_2E12 [MICA/B]_L1, (xiii) SEQ ID Nos: 335 and 339 for D94852_2E12 [MICA/B]_H1_D94852_2E12 [MICA/B]_L2, (xiv) SEQ ID Nos: 343 and 347 for D94852_2E12 [MICA/B]_H2_D94852_2E12 [MICA/B]_L1, (xv) SEQ ID Nos: 351 and 355 for D94852_2E12 [MICA/B]_H2_D94852_2E12 [MICA/B]_L2, (xvi) SEQ ID Nos: 359 and 363 for D99136_2F7 [MICA/B]_H0_D99136_2F7 [MICA/B]_L0, (xvii) SEQ ID Nos: 367 and 371 for D99136_2F7 [MICA/B]_H1_D99136_2F7 [MICA/B]_L1, (xviii) SEQ ID Nos: 375 and 379 for D99136_2F7 [MICA/B]_H1_D99136_2F7 [MICA/B]_L2, (xix) SEQ ID Nos: 383 and 387 for D99136_2F7 [MICA/B]_H2_D99136_2F7 [MICA/B]_L1, (xx) SEQ ID Nos: 391 and 395 for D99136_2F7 [MICA/B]_H2_D99136_2F7 [MICA/B]_L2, (xxi) SEQ ID Nos: 399 and 403 for D103388_1C7 [MICA/B]_H0_D103388_1C7 [MICA/B]_L0, (xxii) SEQ ID Nos: 407 and 411 for D103388_1C7 [MICA/B]_H1_D103388_1C7 [MICA/B]_L1, (xxiii) SEQ ID Nos: 415 and 419 for D103388_1C7 [MICA/B]_H1_D103388_1C7 [MICA/B]_L2, (xxiv) SEQ ID Nos: 423 and 427 for D103388_1C7 [MICA/B]_H2_D103388_1C7 [MICA/B]_L1, (xxv) SEQ ID Nos: 431 and 435 for D103388_1C7 [MICA/B]_H2_D103388_1C7 [MICA/B]_L2, (xxvi) SEQ ID Nos: 439 and 443 for D103388_1D7 [MICA/B]_H0_D103388_1D7 [MICA/B]_L0, (xxvii) SEQ ID Nos: 447 and 451 for D103388_1D7 [MICA/B]_H1_D103388_1D7 [MICA/B]_L1, (xxviii) SEQ ID Nos: 455 and 459 for D103388_1D7 [MICA/B]_H1D103388_1D7 [MICA/B]_L2, (xxix) SEQ ID Nos: 463 and 467 for D103388_1D7 [MICA/B]_H2_D103388_1D7 [MICA/B]_L1, (xxx) SEQ ID Nos: 471 and 475 for D103388_1D7 [MICA/B]_H2_D103388_1D7 [MICA/B]_L2, (xxxi) SEQ ID Nos: 479 and 483 for D105317_1A2 [MICA/B]_H0_D105317_1A2 [MICA/B]_L0, (xxxii) SEQ ID Nos: 487 and 491 for D105317_1A2 [MICA/B]_H1_D105317_1A2 [MICA/B]_L1, (xxxiii) SEQ ID Nos: 495 and 499 for D105317_1A2 [MICA/B]_H1_D105317_1A2 [MICA/B]_L2, (xxxiv) SEQ ID Nos: 503 and 507 for D105317_1A2 [MICA/B]_H2_D105317_1A2 [MICA/B]_L1, (xxxv) SEQ ID Nos: 511 and 515 for D105317_1A2 [MICA/B]_H2_D105317_1A2 [MICA/B]_L2, (xxxvi) SEQ ID Nos: 519 and 523 for D99136_2C11 [MICA/B]_H0_D99136_2C11 [MICA/B]_L0, (xxxvii) SEQ ID Nos: 527 and 531 for D99136_2C11 [MICA/B]_H1_D99136_2C11 [MICA/B]_L1, (xxxviii) SEQ ID Nos: 535 and 539 for D99136_2C11 [MICA/B]_H1_D99136_2C11 [MICA/B]_L2, (xxxix) SEQ ID Nos: 543 and 547 for D99136_2C11 [MICA/B]_H2_D99136_2C11 [MICA/B]_L1, (xl) SEQ ID Nos: 551 and 555 for D99136_2C11 [MICA/B]_H2_D99136_2C11 [MICA/B]_L2, (xli) SEQ ID Nos: 559 and 563 for D103388_1B11-2 [MICA/B]_H0_D103388_1B11-2 [MICA/B]_L0, (xlii) SEQ ID Nos: 567 and 571 for D105317_1B6 [MICA/B]_H0_D105317_1B6 [MICA/B]_L0, (xliii) SEQ ID Nos: 575 and 579 for D105317_1C8 [MICA/B]_H0_D105317_1C8 [MICA/B]_L0, (xliv) SEQ ID Nos: 583 and 587 for D105317_1F6 [MICA/B]_H0_D105317_1F6 [MICA/B]_L0, (xlv) SEQ ID Nos: 591 and 595 for D105317_1F7 [MICA/B]_H0_D105317_1F7 [MICA/B]_L0, (xlvi) SEQ ID Nos: 599 and 603 for D94837_1D3 [MICA/B]_H0_D94837_1D3 [MICA/B]_L0, (xlvii) SEQ ID Nos: 607 and 611 for D94837_1D8 [MICA/B]_H0_D94837_1D8 [MICA/B]_L0, (xlviii) SEQ ID Nos: 615 and 619 for D94837_1D9 [MICA/B]_H0_D94837_1D9 [MICA/B]_L0, (xlix) SEQ ID Nos: 623 and 627 for D94837_1E1 [MICA/B]_H0_D94837_1E1 [MICA/B]_L0, (1) SEQ ID Nos: 631 and 635 for D94852_2D4 [MICA/B]_H0_D94852_2D4 [MICA/B]_L0, (li) SEQ ID Nos: 639 and 643 for D94852_2G8 [MICA/B]_H0_D94852_2G8 [MICA/B]_L0, (lii) SEQ ID Nos: 647 and 651 for D88487_2A8 [MICA/B]_H0_D88487_2A8 [MICA/B]_L0, (liii) SEQ ID Nos: 655 and 659 for D94837_3B12 [MICA/B]_H0_D94837_3B12 [MICA/B]_L0, (liv) SEQ ID Nos: 663 and 667 for D99122_1H7 [MICA/B]_H0_D99122_1H7 [MICA/B]_L0, (lv) SEQ ID Nos: 671 and 675 for D99136_2E8 [MICA/B]_H0_D99136_2E8 [MICA/B]_L0, (lvi) SEQ ID Nos: 647 and 651 for D88487_2A8 [MICA/B]_H0_D88487_2A8 [MICA/B]_L0, (lvii) SEQ ID Nos: 679 and 683 for 3F9[MICA/B]_H0_3F9[MICA/B]_L0, (lviii) SEQ ID Nos: 687 and 691 for 6E1[MICA/B]_H0_6E1[MICA/B]_L0, (lix) SEQ ID Nos: 695 and 699 for 7C6[MICA/B]_H0_7C6[MICA/B]_L0, (lx) SEQ ID Nos: 703 and 707 for 13A9 [MICA/B]_H0_13A9 [MICA/B]_L0, and (lxi) SEQ ID Nos: 711 and 715 for 1D5 [MICA/B]_H0_1D5 [MICA/B]_L0, as depicted in FIGS. 21 and 22.

In a further embodiment and in accordance with any of the above, the anti-NKp46 scFv comprises a set of vhCDR1-3 and vlCDR1-3 from a first variable heavy VH1 domain and a first variable light VL1 domain pair selected from the group including: (i) SEQ ID NOs: 720-722 for vhCDR1-3 and SEQ ID NOs: 724-726 for vlCDR1-3 of 2C10A3.372[NKp46]_H1_2C10A3.372[NKp46]_L1, and (ii) SEQ ID NOs: 728-730 for vhCDR1-3 and SEQ ID NOs: 732-734 for vlCDR1-3 of NKp46-A[NKp46]_H_NKp46-A[NKp46]_L, as depicted in FIGS. 23 and 24.

In a further embodiment and in accordance with any of the above, the anti-NKp46 scFv comprises a variable heavy domain and variable light domain pair selected from the group including: (i) SEQ ID Nos: 719 and 723 for 2C10A3.372[NKp46]_H1_2C10A3.372[NKp46]_L1, and (ii) SEQ ID Nos: 727 and 731 for NKp46-A[NKp46]_H_NKp46-A[NKp46]_L, as depicted in FIGS. 23 and 24.

In a further embodiment and in accordance with any of the above, the variable light domain of the anti-NKp46 scFv is covalently attached to the N-terminus of the first Fc domain using a domain linker, or the variable heavy domain of the anti-NKp46 scFv is covalently attached to the N-terminus of the first Fc domain using a domain linker.

In a further embodiment and in accordance with any of the above, the heterodimeric antibody comprises a first amino acid sequence, a second amino acid sequence, and a third amino acid sequence, wherein the first, second, and third amino acid sequences are selected from the group including: (i) the amino acid sequences of SEQ ID NOs: 809-811 of XENP47438, and (ii) the amino acid sequences of SEQ ID NOs: 812-814 of XENP47439, as depicted in FIG. 33.

In a further embodiment and in accordance with any of the above, the scFv linker is a charged scFv linker.

In a further embodiment and in accordance with the above, the scFv linker is a charged scFv linker having the amino acid sequence (GKPGS)₄ (SEQ ID NO: 1).

In a further embodiment and in accordance with any of the above, the first and second Fc domains are each variant Fc domains.

In a further embodiment and in accordance with the above, the first and/or second variant Fc domains comprise one or more FcγRIIIA (CD16a) binding variant substitutions.

In a further embodiment and in accordance with the above, the one or more FcγRIIIA (CD16a) binding variant substitutions are selected from the group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with the above, the first and second variant Fc domains comprise a set of FcγRIIIA (CD16a) binding variant substitutions selected from the group including: (i) S239D/I332E: S239D/I332E, (ii) S239D: S239D, (iii) 1332E: 1332E, (iv) WT: S239D/I332E, (v) WT: S239D, (vi) WT: 1332E, (vii) S239D/I332E: WT, (viii) S239D: WT, (ix) 1332E: WT, (x) S239D/I332E: S239D, (xi) S239D/I332E: 1332E, (xii) S239D: S239D/I332E, (xiii) 1332E: S239D/I332E, (xiv) S239D: 1332E, and (xv) 1332E: S239D, wherein numbering is according to EU numbering.

In a further embodiment and in accordance with any of the above, the first and/or second variant Fc domains comprise the FcγRIIIA (CD16a) binding variant substitutions of S239D/I332E, wherein numbering is according to EU numbering.

In a further embodiment and in accordance with any of the above, the first and second variant Fc domains comprise a set of heterodimerization variants selected from the group including those depicted in FIGS. 4A-4F, wherein numbering is according to EU numbering.

In a further embodiment and in accordance with the above, the set of heterodimerization variants is selected from the group including: (i) S364K/E357Q: L368D/K370S, (ii) S364K: L368D/K370S, (iii) S364K: L368E/K370S, (iv) D401K: T411E/K360E/Q362E, and (v) T366W: T366S/L368A/Y407V, wherein numbering is according to EU numbering.

In a further embodiment and in accordance with any of the above, the first and second variant Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant Fc domain comprises one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first monomer comprises amino acid variants S364K/E357Q/E233P/L234V/L235A/G236de1/S267K, wherein the second monomer comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second monomers each further comprise amino acid variants M428L/N434S, M428L/434A, or M252Y/S254T/T256E, wherein numbering is according to EU numbering.

In another aspect, a nucleic acid composition comprising nucleic acids encoding the first and second monomers and the light chain of the antibody (as described above) is provided.

In another aspect, an expression vector comprising the nucleic acids (as described above) is provided.

In another aspect, a host cell transformed with the expression vector (as described above) is provided.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, the present disclosure provides a heterodimeric antibody, comprising: (a) a first monomer comprising, from N-terminus to C-terminus, a VH1-CH1-first linker-scFv-second linker-CH2-CH3, wherein the VH1 is a first variable heavy domain, the scFv is an anti-MICA/B scFv, and the CH2-CH3 is a first Fc domain; (b) a second monomer comprising, from N-terminus to C-terminus, a VH2-CH1-hinge-CH2-CH3, wherein the VH2 is a second variable heavy domain and the CH2-CH3 is a second Fc domain; and (c) a common light chain comprising, from N-terminus to C-terminus, a VL-CL, wherein: (i) the VL is a variable light domain and the CL is a light chain constant domain, (ii) the first variable heavy VH1 domain and the variable light VL domain form a first NKp46 antigen binding domain, and (iii) the second variably heavy VH2 domain and the variable light VL domain form a second NKp46 antigen binding domain.

In a further embodiment and in accordance with the above, the first and/or second NKp46 antigen binding domains comprise a set of vhCDR1-3 and vlCDR1-3 from a variable heavy domain and variable light domain pair selected from the group including: (i) SEQ ID NOs: 720-722 for vhCDR1-3 and SEQ ID NOs: 724-726 for vlCDR1-3 of 2C10A3.372[NKp46]_H1_2C10A3.372[NKp46]_L1, and (ii) SEQ ID NOs: 728-730 for vhCDR1-3 and SEQ ID NOs: 732-734 for vlCDR1-3 of NKp46-A[NKp46]_H_NKp46-A[NKp46]_L, as depicted in FIGS. 23 and 24.

In a further embodiment and in accordance with any of the above, the first and/or second NKp46 antigen binding domains comprise a variable heavy domain and variable light domain pair selected from the group including: (i) SEQ ID Nos: 719 and 723 for 2C10A3.372[NKp46]_H1_2C10A3.372[NKp46]_L1, and (ii) SEQ ID Nos: 727 and 731 for NKp46-A[NKp46]_H_NKp46-A[NKp46]_L, as depicted in FIGS. 23 and 24.

In a further embodiment and in accordance with any of the above, the anti-MICA/B scFv comprises a set of vhCDR1-3 and vlCDR1-3 from a first variable heavy VH1 domain and a first variable light VL1 domain pair selected from the group including: (i) SEQ ID NOs: 240-242 for vhCDR1-3 and SEQ ID NOs: 244-246 for vlCDR1-3 of D94837_1E11_1 [MICA/B]_H0_D94837_E11_1 [MICA/B]_L0, (ii) SEQ ID NOs: 248-250 for vhCDR1-3 and SEQ ID NOs: 252-254 for vlCDR1-3 of D94837_E11_1 [MICA/B]_H1_D94837_1E11_1 [MICA/B]_L1, (iii) SEQ ID NOs: 256-258 for vhCDR1-3 and SEQ ID NOs: 260-262 for vlCDR1-3 of D94837_1E11_1 [MICA/B]_H1_D94837_1E11_1 [MICA/B]_L2, (iv) SEQ ID NOs: 264-266 for vhCDR1-3 and SEQ ID NOs: 268-270 for vlCDR1-3 of D94837_1E11_1 [MICA/B]_H2_D94837_1E11_1 [MICA/B]_L1, (v) SEQ ID NOs: 272-274 for vhCDR1-3 and SEQ ID NOs: 276-278 for vlCDR1-3 of D94837_E11_1 [MICA/B]_H2_D94837_1E11_1 [MICA/B]_L2, (vi) SEQ ID NOs: 280-282 for vhCDR1-3 and SEQ ID NOs: 284-286 for vlCDR1-3 of 2E5 [MICA/B]_H0_2E5 [MICA/B]_L0, (vii) SEQ ID NOs: 288-290 for vhCDR1-3 and SEQ ID NOs: 292-294 for v1CDR1-3 of 2E5 [MICA/B]_H1_2E5 [MICA/B]_L1, (viii) SEQ ID NOs: 296-298 for vhCDR1-3 and SEQ ID NOs: 300-302 for v1CDR1-3 of 2E5 [MICA/B]_H1_2E5 [MICA/B]_L2, (ix) SEQ ID NOs: 304-306 for vhCDR1-3 and SEQ ID NOs: 308-310 for v1CDR1-3 of 2E5 [MICA/B]_H2_2E5 [MICA/B]_L1, (x) SEQ ID NOs: 312-314 for vhCDR1-3 and SEQ ID NOs: 316-318 for v1CDR1-3 of 2E5 [MICA/B]_H2_2E5 [MICA/B]_L2, (xi) SEQ ID NOs: 320-322 for vhCDR1-3 and SEQ ID NOs: 324-326 for v1CDR1-3 of D94852_2E12 [MICA/B]_H0_D94852_2E12 [MICA/B]_L0, (xii) SEQ ID NOs: 328-330 for vhCDR1-3 and SEQ ID NOs: 332-334 for v1CDR1-3 of D94852_2E12 [MICA/B]_H1_D94852_2E12 [MICA/B]_L1, (xiii) SEQ ID NOs: 336-338 for vhCDR1-3 and SEQ ID NOs: 340-342 for v1CDR1-3 of D94852_2E12 [MICA/B]_H1_D94852_2E12 [MICA/B]_L2, (xiv) SEQ ID NOs: 344-346 for vhCDR1-3 and SEQ ID NOs: 348-350 for v1CDR1-3 of D94852_2E12 [MICA/B]_H2_D94852_2E12 [MICA/B]_L1, (xv) SEQ ID NOs: 352-354 for vhCDR1-3 and SEQ ID NOs: 356-358 for v1CDR1-3 of D94852_2E12 [MICA/B]_H2_D94852_2E12 [MICA/B]_L2, (xvi) SEQ ID NOs: 360-362 for vhCDR1-3 and SEQ ID NOs: 364-366 for v1CDR1-3 of D99136_2F7 [MICA/B]_H0_D99136_2F7 [MICA/B]_L0, (xvii) SEQ ID NOs: 368-370 for vhCDR1-3 and SEQ ID NOs: 372-374 for v1CDR1-3 of D99136_2F7 [MICA/B]_H1_D99136_2F7 [MICA/B]_L1, (xviii) SEQ ID NOs: 376-378 for vhCDR1-3 and SEQ ID NOs: 380-382 for v1CDR1-3 of D99136_2F7 [MICA/B]_H1_D99136_2F7 [MICA/B]_L2, (xix) SEQ ID NOs: 384-386 for vhCDR1-3 and SEQ ID NOs: 388-390 for v1CDR1-3 of D99136_2F7 [MICA/B]_H2_D99136_2F7 [MICA/B]_L1, (xx) SEQ ID NOs: 392-394 for vhCDR1-3 and SEQ ID NOs: 396-398 for v1CDR1-3 of D99136_2F7 [MICA/B]_H2_D99136_2F7 [MICA/B]_L2, (xxi) SEQ ID NOs: 400-402 for vhCDR1-3 and SEQ ID NOs: 404-406 for v1CDR1-3 of D103388_1C7 [MICA/B]_H0_D103388_1C7 [MICA/B]_L0, (xxii) SEQ ID NOs: 408-410 for vhCDR1-3 and SEQ ID NOs: 412-414 for v1CDR1-3 of D103388_1C7 [MICA/B]_H1_D103388_1C7 [MICA/B]_L1, (xxiii) SEQ ID NOs: 416-418 for vhCDR1-3 and SEQ ID NOs: 420-422 for v1CDR1-3 of D103388_1C7 [MICA/B]_H1_D103388_1C7 [MICA/B]_L2, (xxiv) SEQ ID NOs: 424-426 for vhCDR1-3 and SEQ ID NOs: 428-430 for v1CDR1-3 of D103388_1C7 [MICA/B]_H2_D103388_1C7 [MICA/B]_L1, (xxv) SEQ ID NOs: 432-434 for vhCDR1-3 and SEQ ID NOs: 436-438 for v1CDR1-3 of D103388_1C7 [MICA/B]_H2_D103388_1C7 [MICA/B]_L2, (xxvi) SEQ ID NOs: 440-442 for vhCDR1-3 and SEQ ID NOs: 444-446 for v1CDR1-3 of D103388_1D7 [MICA/B]_H0_D103388_1D7 [MICA/B]_L0, (xxvii) SEQ ID NOs: 448-450 for vhCDR1-3 and SEQ ID NOs: 452-454 for v1CDR1-3 of D103388_1D7 [MICA/B]_H1_D103388_1D7 [MICA/B]_L1, (xxviii) SEQ ID NOs: 456-458 for vhCDR1-3 and SEQ ID NOs: 460-462 for v1CDR1-3 of D103388_1D7 [MICA/B]_H1_D103388_1D7 [MICA/B]_L2, (xxix) SEQ ID NOs: 464-466 for vhCDR1-3 and SEQ ID NOs: 468-470 for v1CDR1-3 of D103388_1D7 [MICA/B]_H2_D103388_1D7 [MICA/B]_L1, (xxx) SEQ ID NOs: 472-474 for vhCDR1-3 and SEQ ID NOs: 476-478 for v1CDR1-3 of D103388_1D7 [MICA/B]_H2_D103388_1D7 [MICA/B]_L2, (xxxi) SEQ ID NOs: 480-482 for vhCDR1-3 and SEQ ID NOs: 484-486 for v1CDR1-3 of D105317_1A2 [MICA/B]_H0_D105317_1A2 [MICA/B]_L0, (xxxii) SEQ ID NOs: 488-490 for vhCDR1-3 and SEQ ID NOs: 492-494 for v1CDR1-3 of D105317_1A2 [MICA/B]_H1_D105317_1A2 [MICA/B]_L1, (xxxiii) SEQ ID NOs: 496-498 for vhCDR1-3 and SEQ ID NOs: 500-502 for v1CDR1-3 of D105317_1A2 [MICA/B]_H1_D105317_1A2 [MICA/B]_L2, (xxxiv) SEQ ID NOs: 504-506 for vhCDR1-3 and SEQ ID NOs: 508-510 for vlCDR1-3 of D105317_1A2 [MICA/B]_H2_D105317_1A2 [MICA/B]_L1, (xxxv) SEQ ID NOs: 512-514 for vhCDR1-3 and SEQ ID NOs: 516-518 for v1CDR1-3 of D105317_1A2 [MICA/B]_H2_D105317_1A2 [MICA/B]_L2, (xxxvi) SEQ ID NOs: 520-522 for vhCDR1-3 and SEQ ID NOs: 524-526 for v1CDR1-3 of D99136_2C11 [MICA/B]_H0_D99136_2C11 [MICA/B]_L0, (xxxvii) SEQ ID NOs: 528-530 for vhCDR1-3 and SEQ ID NOs: 532-534 for vlCDR1-3 of D99136_2C11 [MICA/B]_H1_D99i36_2C11 [MICA/B]_L1, (xxxviii) SEQ ID NOs: 536-538 for vhCDR1-3 and SEQ ID NOs: 540-542 for v1CDR1-3 of D99136_2C11 [MICA/B]_H1_D99136_2C11 [MICA/B]_L2, (xxxix) SEQ ID NOs: 544-546 for vhCDR1-3 and SEQ ID NOs: 548-550 for v1CDR1-3 of D99136_2C11 [MICA/B]_H2_D99136_2C11 [MICA/B]_L1, (xl) SEQ ID NOs: 552-554 for vhCDR1-3 and SEQ ID NOs: 556-558 for v1CDR1-3 of D99136_2C11 [MICA/B]_H2_D99136_2C11 [MICA/B]_L2, (xli) SEQ ID NOs: 560-562 for vhCDR1-3 and SEQ ID NOs: 564-566 for vlCDR1-3 of D103388_1B11-2 [MICA/B]_H0_D103388_1B11-2 [MICA/B]_L0, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (lvii) SEQ ID NOs: 680-682 for vhCDR1-3 and SEQ ID NOs: 684-686 for vlCDR1-3 of 3F9[MICA/B]_H0_3F9[MICA/B]_L0, (lviii) SEQ ID NOs: 688-690 for vhCDR1-3 and SEQ ID NOs: 692-694 for vlCDR1-3 of 6E1[MICA/B]_H0_6E1[MICA/B]_L0, (lix) SEQ ID NOs: 696-698 for vhCDR1-3 and SEQ ID NOs: 700-702 for vlCDR1-3 of 7C6[MICA/B]_H0_7C6[MICA/B]_L0, (lx) SEQ ID NOs: 704-706 for vhCDR1-3 and SEQ ID NOs: 708-710 for vlCDR1-3 of 13A9 [MICA/B]_H0_13A9 [MICA/B]_L0, and (lxi) SEQ ID NOs: 712-714 for vhCDR1-3 and SEQ ID NOs: 716-718 for vlCDR1-3 of 1D5 [MICA/B]_H0_1D5 [MICA/B]_L0, as depicted in FIGS. 21 and 22.

In a further embodiment and in accordance with any of the above, the anti-MICA/B scFv comprises a variable heavy domain and variable light domain pair selected from the group including: (i) SEQ ID Nos: 239 and 243 for D94837_E11_1 [MICA/B]_H0_D94837_1E11_1 [MICA/B]_L0, (ii) SEQ ID Nos: 247 and 251 for D94837_1E11_1 [MICA/B]_H1_D94837_1E11_1 [MICA/B]_L1, (iii) SEQ ID Nos: 255 and 259 for D94837_1E11_1 [MICA/B]_H1_D94837_E11_1 [MICA/B]_L2, (iv) SEQ ID Nos: 263 and 267 for D94837_1E11_1 [MICA/B]_H2_D94837_1E11_1 [MICA/B]_L1, (v) SEQ ID Nos: 271 and 275 for D94837_E11_1 [MICA/B]_H2_D94837_E11_1 [MICA/B]_L2, (vi) SEQ ID Nos: 279 and 283 for 2E5 [MICA/B]_H0_2E5 [MICA/B]_L0, (vii) SEQ ID Nos: 287 and 291 for 2E5 [MICA/B]_H1_2E5 [MICA/B]_L1, (viii) SEQ ID Nos: 295 and 299 for 2E5 [MICA/B]_H1_2E5 [MICA/B]_L2, (ix) SEQ ID Nos: 303 and 307 for 2E5 [MICA/B]_H2_2E5 [MICA/B]_L1, (x) SEQ ID Nos: 311 and 315 for 2E5 [MICA/B]_H2_2E5 [MICA/B]_L2, (xi) SEQ ID Nos: 319 and 323 for D94852_2E12 [MICA/B]_H0_D94852_2E12 [MICA/B]_L0, (xii) SEQ ID Nos: 327 and 331 for D94852_2E12 [MICA/B]_H1_D94852_2E12 [MICA/B]_L1, (xiii) SEQ ID Nos: 335 and 339 for D94852_2E12 [MICA/B]_H1_D94852_2E12 [MICA/B]_L2, (xiv) SEQ ID Nos: 343 and 347 for D94852_2E12 [MICA/B]_H2_D94852_2E12 [MICA/B]_L1, (xv) SEQ ID Nos: 351 and 355 for D94852_2E12 [MICA/B]_H2_D94852_2E12 [MICA/B]_L2, (xvi) SEQ ID Nos: 359 and 363 for D99136_2F7 [MICA/B]_H0_D99136_2F7 [MICA/B]_L0, (xvii) SEQ ID Nos: 367 and 371 for D99136_2F7 [MICA/B]_H1_D99136_2F7 [MICA/B]_L1, (xviii) SEQ ID Nos: 375 and 379 for D99136_2F7 [MICA/B]_H1_D99136_2F7 [MICA/B]_L2, (xix) SEQ ID Nos: 383 and 387 for D99136_2F7 [MICA/B]_H2_D99136_2F7 [MICA/B]_L1, (xx) SEQ ID Nos: 391 and 395 for D99136_2F7 [MICA/B]_H2_D99136_2F7 [MICA/B]_L2, (xxi) SEQ ID Nos: 399 and 403 for D103388_1C7 [MICA/B]_H0_D103388_1C7 [MICA/B]_L0, (xxii) SEQ ID Nos: 407 and 411 for D103388_1C7 [MICA/B]_H1_D103388_1C7 [MICA/B]_L1, (xxiii) SEQ ID Nos: 415 and 419 for D103388_1C7 [MICA/B]_H1_D103388_1C7 [MICA/B]_L2, (xxiv) SEQ ID Nos: 423 and 427 for D103388_1C7 [MICA/B]_H2_D103388_1C7 [MICA/B]_L1, (xxv) SEQ ID Nos: 431 and 435 for D103388_1C7 [MICA/B]_H2_D103388_1C7 [MICA/B]_L2, (xxvi) SEQ ID Nos: 439 and 443 for D103388_1D7 [MICA/B]_H0_D103388_1D7 [MICA/B]_L0, (xxvii) SEQ ID Nos: 447 and 451 for D103388_1D7 [MICA/B]_H1_D103388_1D7 [MICA/B]_L1, (xxviii) SEQ ID Nos: 455 and 459 for D103388_1D7 [MICA/B]_H1_D103388_1D7 [MICA/B]_L2, (xxix) SEQ ID Nos: 463 and 467 for D103388_1D7 [MICA/B]_H2_D103388_1D7 [MICA/B]_L1, (xxx) SEQ ID Nos: 471 and 475 for D103388_1D7 [MICA/B]_H2_D103388_1D7 [MICA/B]_L2, (xxxi) SEQ ID Nos: 479 and 483 for D105317_1A2 [MICA/B]_H0_D105317_1A2 [MICA/B]_L0, (xxxii) SEQ ID Nos: 487 and 491 for D105317_1A2 [MICA/B]_H1_D105317_1A2 [MICA/B]_L1, (xxxiii) SEQ ID Nos: 495 and 499 for D105317_1A2 [MICA/B]_H1_D105317_1A2 [MICA/B]_L2, (xxxiv) SEQ ID Nos: 503 and 507 for D105317_1A2 [MICA/B]_H2_D105317_1A2 [MICA/B]_L1, (xxxv) SEQ ID Nos: 511 and 515 for D105317_1A2 [MICA/B]_H2_D105317_1A2 [MICA/B]_L2, (xxxvi) SEQ ID Nos: 519 and 523 for D99136_2C11 [MICA/B]_H0_D99136_2C11 [MICA/B]_L0, (xxxvii) SEQ ID Nos: 527 and 531 for D99136_2C11 [MICA/B]_H1_D99136_2C11 [MICA/B]_L1, (xxxviii) SEQ ID Nos: 535 and 539 for D99136_2C11 [MICA/B]_H1_D99136_2C11 [MICA/B]_L2, (xxxix) SEQ ID Nos: 543 and 547 for D99136_2C11 [MICA/B]_H2_D99136_2C11 [MICA/B]_L1, (xl) SEQ ID Nos: 551 and 555 for D99136_2C11 [MICA/B]_H2_D99136_2C11 [MICA/B]_L2, (xli) SEQ ID Nos: 559 and 563 for D103388_1B11-2 [MICA/B]_H0_D103388_1B11-2 [MICA/B]_L0, (xlii) SEQ ID Nos: 567 and 571 for D105317_1B6 [MICA/B]_H0_D105317_1B6 [MICA/B]_L0, (xliii) SEQ ID Nos: 575 and 579 for D105317_1C8 [MICA/B]_H0_D105317_1C8 [MICA/B]_L0, (xliv) SEQ ID Nos: 583 and 587 for D105317_1F6 [MICA/B]_H0_D105317_1F6 [MICA/B]_L0, (xlv) SEQ ID Nos: 591 and 595 for D105317_1F7 [MICA/B]_H0_D105317_1F7 [MICA/B]_L0, (xlvi) SEQ ID Nos: 599 and 603 for D94837_1D3 [MICA/B]_H0_D94837_1D3 [MICA/B]_L0, (xlvii) SEQ ID Nos: 607 and 611 for D94837_1D8 [MICA/B]_H0_D94837_1D8 [MICA/B]_L0, (xlviii) SEQ ID Nos: 615 and 619 for D94837_1D9 [MICA/B]_H0_D94837_1D9 [MICA/B]_L0, (xlix) SEQ ID Nos: 623 and 627 for D94837_1E1 [MICA/B]_H0_D94837_1E1 [MICA/B]_L0, (1) SEQ ID Nos: 631 and 635 for D94852_2D4 [MICA/B]_H0_D94852_2D4 [MICA/B]_L0, (li) SEQ ID Nos: 639 and 643 for D94852_2G8 [MICA/B]_H0_D94852_2G8 [MICA/B]_L0, (lii) SEQ ID Nos: 647 and 651 for D88487_2A8 [MICA/B]_H0_D88487_2A8 [MICA/B]_L0, (liii) SEQ ID Nos: 655 and 659 for D94837_3B12 [MICA/B]_H0_D94837_3B12 [MICA/B]_L0, (liv) SEQ ID Nos: 663 and 667 for D99122_1H7 [MICA/B]_H0_D99122_1H7 [MICA/B]_L0, (lv) SEQ ID Nos: 671 and 675 for D99136_2E8 [MICA/B]_H0_D99136_2E8 [MICA/B]_L0, (lvi) SEQ ID Nos: 647 and 651 for D88487_2A8 [MICA/B]_H0_D88487_2A8 [MICA/B]_L0, (lvii) SEQ ID Nos: 679 and 683 for 3F9[MICA/B]_H0_3F9[MICA/B]_L0, (lviii) SEQ ID Nos: 687 and 691 for 6E1[MICA/B]_H0_6E1[MICA/B]_L0, (lix) SEQ ID Nos: 695 and 699 for 7C6[MICA/B]_H0_7C6[MICA/B]_L0, (lx) SEQ ID Nos: 703 and 707 for 13A9 [MICA/B]_H0_13A9 [MICA/B]_L0, and (lxi) SEQ ID Nos: 711 and 715 for 1D5 [MICA/B]_H0_1D5 [MICA/B]_L0, as depicted in FIGS. 21 and 22.

In a further embodiment and in accordance with any of the above, the variable light domain of the anti-MICA/B scFv is covalently attached to the N-terminus of the first Fc domain using a domain linker, or the variable heavy domain of the anti-MICA/B scFv is covalently attached to the N-terminus of the first Fc domain using a domain linker.

In a further embodiment and in accordance with any of the above, the scFv linker is a charged scFv linker.

In a further embodiment and in accordance with the above, the scFv linker is a charged scFv linker having the amino acid sequence (GKPGS)₄ (SEQ ID NO: 1).

In a further embodiment and in accordance with any of the above, the first and second Fc domains are each variant Fc domains.

In a further embodiment and in accordance with the above, the first and/or second variant Fc domains comprise one or more FcγRIIIA (CD16a) binding variant substitutions.

In a further embodiment and in accordance with the above, the one or more FcγRIIIA (CD16a) binding variant substitutions are selected from the group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with the above, the first and second variant Fc domains comprise a set of FcγRIIIA (CD16a) binding variant substitutions selected from the group including: (i) S239D/I332E: S239D/I332E, (ii) S239D: S239D, (iii) 1332E: 1332E, (iv) WT: S239D/I332E, (v) WT: S239D, (vi) WT: 1332E, (vii) S239D/I332E: WT, (viii) S239D: WT, (ix) 1332E: WT, (x) S239D/I332E: S239D, (xi) S239D/I332E: 1332E, (xii) S239D: S239D/I332E, (xiii) 1332E: S239D/I332E, (xiv) S239D: 1332E, and (xv) 1332E: S239D, wherein numbering is according to EU numbering.

In a further embodiment and in accordance with any of the above, the first and/or second variant Fc domains comprise the FcγRIIIA (CD16a) binding variant substitutions of S239D/I332E, wherein numbering is according to EU numbering.

In a further embodiment and in accordance with any of the above, the first and second variant Fc domains comprise a set of heterodimerization variants selected from the group including those depicted in FIGS. 4A-4F, wherein numbering is according to EU numbering.

In a further embodiment and in accordance with the above, the set of heterodimerization variants is selected from the group including: (i) S364K/E357Q: L368D/K370S, (ii) S364K: L368D/K370S, (iii) S364K: L368E/K370S, (iv) D401K: T411E/K360E/Q362E, and (v) T366W: T366S/L368A/Y407V, wherein numbering is according to EU numbering.

In a further embodiment and in accordance with any of the above, the first and second variant Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant Fc domain comprises one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first monomer comprises amino acid variants S364K/E357Q/E233P/L234V/L235A/G236del/S267K, wherein the second monomer comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second monomers each further comprise amino acid variants M428L/N434S, M428L/434A, or M252Y/S254T/T256E, wherein numbering is according to EU numbering.

In another aspect, a nucleic acid composition comprising nucleic acids encoding the first and second monomers and the light chain of the antibody (as described above) is provided.

In another aspect, an expression vector comprising the nucleic acids (as described above) is provided.

In another aspect, a host cell transformed with the expression vector (as described above) is provided.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, the present disclosure provides a heterodimeric antibody, comprising: (a) a first monomer comprising, from N-terminus to C-terminus, a VH1-CH1-hinge-first linker-VH1-CH1-hinge-CH2-CH3, wherein VH1 is a first variable heavy domain and CH2-CH3 is a first Fc domain; (b) a light chain comprising, from N-terminus to C-terminus, a VL1-CL, wherein VL1 is a first variable light domain and CL is a constant light domain, and wherein the VH1 and VL1 form MICA/B antigen binding domains; and (c) a second monomer comprising, from N-terminus to C-terminus, an anti-NKp46 scFv and a second Fc domain, wherein the scFv is covalently attached to the N-terminus of the second Fc domain using a domain linker.

In a further embodiment and in accordance with the above, the anti-NKp46 scFv comprises a second variable heavy VH2 domain, an scFv linker, and a second variable light VL2 domain.

In a further embodiment and in accordance with any of the above, the anti-NKp46 scFv comprises a set of vhCDR1-3 and vlCDR1-3 from a variable heavy domain and variable light domain pair selected from the group including: (i) SEQ ID NOs: 720-722 for vhCDR1-3 and SEQ ID NOs: 724-726 for vlCDR1-3 of 2C10A3.372[NKp46]_H1_2C10A3.372[NKp46]_L1, and (ii) SEQ ID NOs: 728-730 for vhCDR1-3 and SEQ ID NOs: 732-734 for vlCDR1-3 of NKp46-A[NKp46]_H_NKp46-A[NKp46]_L, as depicted in FIGS. 23 and 24.

In a further embodiment and in accordance with any of the above, the anti-NKp46 scFv comprises a variable heavy domain and variable light domain pair selected from the group including: (i) SEQ ID Nos: 719 and 723 for 2C10A3.372[NKp46]_H1_2C10A3.372[NKp46]_L1, and (ii) SEQ ID Nos: 727 and 731 for NKp46-A[NKp46]_H_NKp46-A[NKp46]_L, as depicted in FIGS. 23 and 24.

In a further embodiment and in accordance with any of the above, each of the MICA/B antigen binding domains comprise a set of vhCDR1-3 and vlCDR1-3 from a variable heavy domain and variable light domain pair, wherein the set of vhCDR1-3 and vlCDR1-3 is selected from the group including: (i) SEQ ID NOs: 240-242 for vhCDR1-3 and SEQ ID NOs: 244-246 for vlCDR1-3 of D94837_1E11_1 [MICA/B]_H0_D94837_E11_1 [MICA/B]_L0, (ii) SEQ ID NOs: 248-250 for vhCDR1-3 and SEQ ID NOs: 252-254 for vlCDR1-3 of D94837_1E11_1 [MICA/B]_H1_D94837_1E11_1 [MICA/B]_L1, (iii) SEQ ID NOs: 256-258 for vhCDR1-3 and SEQ ID NOs: 260-262 for vlCDR1-3 of D94837_E11_1 [MICA/B]_H1_D94837_1E11_1 [MICA/B]_L2, (iv) SEQ ID NOs: 264-266 for vhCDR1-3 and SEQ ID NOs: 268-270 for vlCDR1-3 of D94837_E11_1 [MICA/B]_H2_D94837_1E11_1 [MICA/B]_L1, (v) SEQ ID NOs: 272-274 for vhCDR1-3 and SEQ ID NOs: 276-278 for vlCDR1-3 of D94837_1E11_1 [MICA/B]_H2_D94837_E11_1 [MICA/B]_L2, (vi) SEQ ID NOs: 280-282 for vhCDR1-3 and SEQ ID NOs: 284-286 for vlCDR1-3 of 2E5 [MICA/B]_H0_2E5 [MICA/B]_L0, (vii) SEQ ID NOs: 288-290 for vhCDR1-3 and SEQ ID NOs: 292-294 for vlCDR1-3 of 2E5 [MICA/B]_H1_2E5 [MICA/B]_L1, (viii) SEQ ID NOs: 296-298 for vhCDR1-3 and SEQ ID NOs: 300-302 for vlCDR1-3 of 2E5 [MICA/B]_H1_2E5 [MICA/B]_L2, (ix) SEQ ID NOs: 304-306 for vhCDR1-3 and SEQ ID NOs: 308-310 for vlCDR1-3 of 2E5 [MICA/B]_H2_2E5 [MICA/B]_L1, (x) SEQ ID NOs: 312-314 for vhCDR1-3 and SEQ ID NOs: 316-318 for vlCDR1-3 of 2E5 [MICA/B]_H2_2E5 [MICA/B]_L2, (xi) SEQ ID NOs: 320-322 for vhCDR1-3 and SEQ ID NOs: 324-326 for vlCDR1-3 of D94852_2E12 [MICA/B]_H0_D94852_2E12 [MICA/B]_L0, (xii) SEQ ID NOs: 328-330 for vhCDR1-3 and SEQ ID NOs: 332-334 for vlCDR1-3 of D94852_2E12 [MICA/B]_H1_D94852_2E12 [MICA/B]_L1, (xiii) SEQ ID NOs: 336-338 for vhCDR1-3 and SEQ ID NOs: 340-342 for vlCDR1-3 of D94852_2E12 [MICA/B]_H1_D94852_2E12 [MICA/B]_L2, (xiv) SEQ ID NOs: 344-346 for vhCDR1-3 and SEQ ID NOs: 348-350 for vlCDR1-3 of D94852_2E12 [MICA/B]_H2_D94852_2E12 [MICA/B]_L1, (xv) SEQ ID NOs: 352-354 for vhCDR1-3 and SEQ ID NOs: 356-358 for vlCDR1-3 of D94852_2E12 [MICA/B]_H2_D94852_2E12 [MICA/B]_L2, (xvi) SEQ ID NOs: 360-362 for vhCDR1-3 and SEQ ID NOs: 364-366 for vlCDR1-3 of D99136_2F7 [MICA/B]_H0_D99136_2F7 [MICA/B]_L0, (xvii) SEQ ID NOs: 368-370 for vhCDR1-3 and SEQ ID NOs: 372-374 for v1CDR1-3 of D99136_2F7 [MICA/B]_H1_D99136_2F7 [MICA/B]_L1, (xviii) SEQ ID NOs: 376-378 for vhCDR1-3 and SEQ ID NOs: 380-382 for v1CDR1-3 of D99136_2F7 [MICA/B]_H1_D99136_2F7 [MICA/B]_L2, (xix) SEQ ID NOs: 384-386 for vhCDR1-3 and SEQ ID NOs: 388-390 for v1CDR1-3 of D99136_2F7 [MICA/B]_H2_D99136_2F7 [MICA/B]_L1, (xx) SEQ ID NOs: 392-394 for vhCDR1-3 and SEQ ID NOs: 396-398 for v1CDR1-3 of D99136_2F7 [MICA/B]_H2_D99136_2F7 [MICA/B]_L2, (xxi) SEQ ID NOs: 400-402 for vhCDR1-3 and SEQ ID NOs: 404-406 for v1CDR1-3 of D103388_1C7 [MICA/B]_H0_D103388_1C7 [MICA/B]_L0, (xxii) SEQ ID NOs: 408-410 for vhCDR1-3 and SEQ ID NOs: 412-414 for v1CDR1-3 of D103388_1C7 [MICA/B]_H1_D103388_1C7 [MICA/B]_L1, (xxiii) SEQ ID NOs: 416-418 for vhCDR1-3 and SEQ ID NOs: 420-422 for v1CDR1-3 of D103388_1C7 [MICA/B]_H1_D103388_1C7 [MICA/B]_L2, (xxiv) SEQ ID NOs: 424-426 for vhCDR1-3 and SEQ ID NOs: 428-430 for v1CDR1-3 of D103388_1C7 [MICA/B]_H2_D103388_1C7 [MICA/B]_L1, (xxv) SEQ ID NOs: 432-434 for vhCDR1-3 and SEQ ID NOs: 436-438 for v1CDR1-3 of D103388_1C7 [MICA/B]_H2_D103388_1C7 [MICA/B]_L2, (xxvi) SEQ ID NOs: 440-442 for vhCDR1-3 and SEQ ID NOs: 444-446 for v1CDR1-3 of D103388_1D7 [MICA/B]_H0_D103388_1D7 [MICA/B]_L0, (xxvii) SEQ ID NOs: 448-450 for vhCDR1-3 and SEQ ID NOs: 452-454 for v1CDR1-3 of D103388_1D7 [MICA/B]_H1_D103388_1D7 [MICA/B]_L1, (xxviii) SEQ ID NOs: 456-458 for vhCDR1-3 and SEQ ID NOs: 460-462 for v1CDR1-3 of D103388_1D7 [MICA/B]_H1_D103388_1D7 [MICA/B]_L2, (xxix) SEQ ID NOs: 464-466 for vhCDR1-3 and SEQ ID NOs: 468-470 for v1CDR1-3 of D103388_1D7 [MICA/B]_H2_D103388_1D7 [MICA/B]_L1, (xxx) SEQ ID NOs: 472-474 for vhCDR1-3 and SEQ ID NOs: 476-478 for v1CDR1-3 of D103388_1D7 [MICA/B]_H2_D103388_1D7 [MICA/B]_L2, (xxxi) SEQ ID NOs: 480-482 for vhCDR1-3 and SEQ ID NOs: 484-486 for v1CDR1-3 of D105317_1A2 [MICA/B]_H0_D105317_1A2 [MICA/B]_L0, (xxxii) SEQ ID NOs: 488-490 for vhCDR1-3 and SEQ ID NOs: 492-494 for v1CDR1-3 of D105317_1A2 [MICA/B]_H1_D105317_1A2 [MICA/B]_L1, (xxxiii) SEQ ID NOs: 496-498 for vhCDR1-3 and SEQ ID NOs: 500-502 for v1CDR1-3 of D105317_1A2 [MICA/B]_H1_D105317_1A2 [MICA/B]_L2, (xxxiv) SEQ ID NOs: 504-506 for vhCDR1-3 and SEQ ID NOs: 508-510 for vlCDR1-3 of D105317_1A2 [MICA/B]_H2_D105317_1A2 [MICA/B]_L1, (xxxv) SEQ ID NOs: 512-514 for vhCDR1-3 and SEQ ID NOs: 516-518 for vlCDR1-3 of D105317_1A2 [MICA/B]_H2_D105317_1A2 [MICA/B]_L2, (xxxvi) SEQ ID NOs: 520-522 for vhCDR1-3 and SEQ ID NOs: 524-526 for v1CDR1-3 of D99136_2C11 [MICA/B]_H0_D99136_2C11 [MICA/B]_L0, (xxxvii) SEQ ID NOs: 528-530 for vhCDR1-3 and SEQ ID NOs: 532-534 for vlCDR1-3 of D99136_2C11 [MICA/B]_H1_D99136_2C11 [MICA/B]_L1, (xxxviii) SEQ ID NOs: 536-538 for vhCDR1-3 and SEQ ID NOs: 540-542 for vlCDR1-3 of D99136_2C11 [MICA/B]_H1_D99136_2C11 [MICA/B]_L2, (xxxix) SEQ ID NOs: 544-546 for vhCDR1-3 and SEQ ID NOs: 548-550 for v1CDR1-3 of D99136_2C11 [MICA/B]_H2_D99136_2C11 [MICA/B]_L1, (xl) SEQ ID NOs: 552-554 for vhCDR1-3 and SEQ ID NOs: 556-558 for v1CDR1-3 of D99136_2C11 [MICA/B]_H2_D99136_2C11 [MICA/B]_L2, (xli) SEQ ID NOs: 560-562 for vhCDR1-3 and SEQ ID NOs: 564-566 for v1CDR1-3 of D103388_1B11-2 [MICA/B]_H0_D103388_1B11-2 [MICA/B]_L0, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (lvii) SEQ ID NOs: 680-682 for vhCDR1-3 and SEQ ID NOs: 684-686 for vlCDR1-3 of 3F9[MICA/B]_H0_3F9[MICA/B]_L0, (lviii) SEQ ID NOs: 688-690 for vhCDR1-3 and SEQ ID NOs: 692-694 for vlCDR1-3 of 6E1[MICA/B]_H0_6E1[MICA/B]_L0, (lix) SEQ ID NOs: 696-698 for vhCDR1-3 and SEQ ID NOs: 700-702 for vlCDR1-3 of 7C6[MICA/B]_H0_7C6[MICA/B]_L0, (lx) SEQ ID NOs: 704-706 for vhCDR1-3 and SEQ ID NOs: 708-710 for vlCDR1-3 of 13A9 [MICA/B]_H0_13A9 [MICA/B]_L0, and (lxi) SEQ ID NOs: 712-714 for vhCDR1-3 and SEQ ID NOs: 716-718 for vlCDR1-3 of 1D5 [MICA/B]_H0_1D5 [MICA/B]_L0, as depicted in FIGS. 21 and 22.

In a further embodiment and in accordance with any of the above, each of the MICA/B antigen binding domains comprise a variable heavy domain and variable light domain pair selected from the group including: (i) SEQ ID Nos: 239 and 243 for D94837_1E11_1 [MICA/B]_H0_D94837_1E11_1 [MICA/B]_L0, (ii) SEQ ID Nos: 247 and 251 for D94837_1E1_1 [MICA/B]_H1_D94837_E11_1 [MICA/B]_L1, (iii) SEQ ID Nos: 255 and 259 for D94837_1E11_1 [MICA/B]_H1_D94837_1E11_1 [MICA/B]_L2, (iv) SEQ ID Nos: 263 and 267 for D94837_E11_1 [MICA/B]_H2_D94837_E11_1 [MICA/B]_L1, (v) SEQ ID Nos: 271 and 275 for D94837_E11_1 [MICA/B]_H2_D94837_E11_1 [MICA/B]_L2, (vi) SEQ ID Nos: 279 and 283 for 2E5 [MICA/B]_H0_2E5 [MICA/B]_L0, (vii) SEQ ID Nos: 287 and 291 for 2E5 [MICA/B]_H1_2E5 [MICA/B]_L1, (viii) SEQ ID Nos: 295 and 299 for 2E5 [MICA/B]_H1_2E5 [MICA/B]_L2, (ix) SEQ ID Nos: 303 and 307 for 2E5 [MICA/B]_H2_2E5 [MICA/B]_L1, (x) SEQ ID Nos: 311 and 315 for 2E5 [MICA/B]_H2_2E5 [MICA/B]_L2, (xi) SEQ ID Nos: 319 and 323 for D94852_2E12 [MICA/B]_H0_D94852_2E12 [MICA/B]_L0, (xii) SEQ ID Nos: 327 and 331 for D94852_2E12 [MICA/B]_H1_D94852_2E12 [MICA/B]_L1, (xiii) SEQ ID Nos: 335 and 339 for D94852_2E12 [MICA/B]_H1_D94852_2E12 [MICA/B]_L2, (xiv) SEQ ID Nos: 343 and 347 for D94852_2E12 [MICA/B]_H2_D94852_2E12 [MICA/B]_L1, (xv) SEQ ID Nos: 351 and 355 for D94852_2E12 [MICA/B]_H2_D94852_2E12 [MICA/B]_L2, (xvi) SEQ ID Nos: 359 and 363 for D99136_2F7 [MICA/B]_H0_D99136_2F7 [MICA/B]_L0, (xvii) SEQ ID Nos: 367 and 371 for D99136_2F7 [MICA/B]_H1_D99136_2F7 [MICA/B]_L1, (xviii) SEQ ID Nos: 375 and 379 for D99136_2F7 [MICA/B]_H1_D99136_2F7 [MICA/B]_L2, (xix) SEQ ID Nos: 383 and 387 for D99136_2F7 [MICA/B]_H2_D99136_2F7 [MICA/B]_L1, (xx) SEQ ID Nos: 391 and 395 for D99136_2F7 [MICA/B]_H2_D99136_2F7 [MICA/B]_L2, (xxi) SEQ ID Nos: 399 and 403 for D103388_1C7 [MICA/B]_H0_D103388_1C7 [MICA/B]_L0, (xxii) SEQ ID Nos: 407 and 411 for D103388_1C7 [MICA/B]_H1_D103388_1C7 [MICA/B]_L1, (xxiii) SEQ ID Nos: 415 and 419 for D103388_1C7 [MICA/B]_H1_D103388_1C7 [MICA/B]_L2, (xxiv) SEQ ID Nos: 423 and 427 for D103388_1C7 [MICA/B]_H2_D103388_1C7 [MICA/B]_L1, (xxv) SEQ ID Nos: 431 and 435 for D103388_1C7 [MICA/B]_H2_D103388_1C7 [MICA/B]_L2, (xxvi) SEQ ID Nos: 439 and 443 for D103388_1D7 [MICA/B]_H0_D103388_1D7 [MICA/B]_L0, (xxvii) SEQ ID Nos: 447 and 451 for D103388_1D7 [MICA/B]_H1_D103388_1D7 [MICA/B]_L1, (xxviii) SEQ ID Nos: 455 and 459 for D103388_1D7 [MICA/B]_H1D103388_1D7 [MICA/B]_L2, (xxix) SEQ ID Nos: 463 and 467 for D103388_1D7 [MICA/B]_H2_D103388_1D7 [MICA/B]_L1, (xxx) SEQ ID Nos: 471 and 475 for D103388_1D7 [MICA/B]_H2_D103388_1D7 [MICA/B]_L2, (xxxi) SEQ ID Nos: 479 and 483 for D105317_1A2 [MICA/B]_H0_D105317_1A2 [MICA/B]_L0, (xxxii) SEQ ID Nos: 487 and 491 for D105317_1A2 [MICA/B]_H1_D105317_1A2 [MICA/B]_L1, (xxxiii) SEQ ID Nos: 495 and 499 for D105317_1A2 [MICA/B]_H1_D105317_1A2 [MICA/B]_L2, (xxxiv) SEQ ID Nos: 503 and 507 for D105317_1A2 [MICA/B]_H2_D105317_1A2 [MICA/B]_L1, (xxxv) SEQ ID Nos: 511 and 515 for D105317_1A2 [MICA/B]_H2_D105317_1A2 [MICA/B]_L2, (xxxvi) SEQ ID Nos: 519 and 523 for D99136_2C11 [MICA/B]_H0_D99136_2C11 [MICA/B]_L0, (xxxvii) SEQ ID Nos: 527 and 531 for D99136_2C11 [MICA/B]_H1_D99136_2C11 [MICA/B]_L1, (xxxviii) SEQ ID Nos: 535 and 539 for D99136_2C11 [MICA/B]_H1_D99136_2C11 [MICA/B]_L2, (xxxix) SEQ ID Nos: 543 and 547 for D99136_2C11 [MICA/B]_H2_D99136_2C11 [MICA/B]_L1, (xl) SEQ ID Nos: 551 and 555 for D99136_2C11 [MICA/B]_H2_D99136_2C11 [MICA/B]_L2, (xli) SEQ ID Nos: 559 and 563 for D103388_1B11-2 [MICA/B]_H0_D103388_1B11-2 [MICA/B]_L0, (xlii) SEQ ID Nos: 567 and 571 for D105317_1B6 [MICA/B]_H0_D105317_1B6 [MICA/B]_L0, (xliii) SEQ ID Nos: 575 and 579 for D105317_1C8 [MICA/B]_H0_D105317_1C8 [MICA/B]_L0, (xliv) SEQ ID Nos: 583 and 587 for D105317_1F6 [MICA/B]_H0_D105317_1F6 [MICA/B]_L0, (xlv) SEQ ID Nos: 591 and 595 for D105317_1F7 [MICA/B]_H0_D105317_1F7 [MICA/B]_L0, (xlvi) SEQ ID Nos: 599 and 603 for D94837_1D3 [MICA/B]_H0_D94837_1D3 [MICA/B]_L0, (xlvii) SEQ ID Nos: 607 and 611 for D94837_1D8 [MICA/B]_H0_D94837_1D8 [MICA/B]_L0, (xlviii) SEQ ID Nos: 615 and 619 for D94837_1D9 [MICA/B]_H0_D94837_1D9 [MICA/B]_L0, (xlix) SEQ ID Nos: 623 and 627 for D94837_1E1 [MICA/B]_H0_D94837_1E1 [MICA/B]_L0, (1) SEQ ID Nos: 631 and 635 for D94852_2D4 [MICA/B]_H0_D94852_2D4 [MICA/B]_L0, (li) SEQ ID Nos: 639 and 643 for D94852_2G8 [MICA/B]_H0_D94852_2G8 [MICA/B]_L0, (lii) SEQ ID Nos: 647 and 651 for D88487_2A8 [MICA/B]_H0_D88487_2A8 [MICA/B]_L0, (liii) SEQ ID Nos: 655 and 659 for D94837_3B12 [MICA/B]_H0_D94837_3B12 [MICA/B]_L0, (liv) SEQ ID Nos: 663 and 667 for D99122_1H7 [MICA/B]_H0_D99122_1H7 [MICA/B]_L0, (lv) SEQ ID Nos: 671 and 675 for D99136_2E8 [MICA/B]_H0_D99136_2E8 [MICA/B]_L0, (lvi) SEQ ID Nos: 647 and 651 for D88487_2A8 [MICA/B]_H0_D88487_2A8 [MICA/B]_L0, (lvii) SEQ ID Nos: 679 and 683 for 3F9[MICA/B]_H0_3F9[MICA/B]_L0, (lviii) SEQ ID Nos: 687 and 691 for 6E1[MICA/B]_H0_6E1[MICA/B]_L0, (lix) SEQ ID Nos: 695 and 699 for 7C6[MICA/B]_H0_7C6[MICA/B]_L0, (lx) SEQ ID Nos: 703 and 707 for 13A9 [MICA/B]_H0_13A9 [MICA/B]_L0, and (lxi) SEQ ID Nos: 711 and 715 for 1D5 [MICA/B]_H0_1D5 [MICA/B]_L0, as depicted in FIGS. 21 and 22.

In a further embodiment and in accordance with any of the above, the heterodimeric antibody comprises a first amino acid sequence, a second amino acid sequence, and a third amino acid sequence, wherein the first, second, and third amino acid sequences are selected from the group including: (i) the amino acid sequences of SEQ ID NOs: 815-817 of XENP47442, and (ii) the amino acid sequences of SEQ ID NOs: 818-820 of XENP47443, as depicted in FIG. 34.

In a further embodiment and in accordance with any of the above, the scFv linker is a charged scFv linker.

In a further embodiment and in accordance with the above, the scFv linker is a charged scFv linker having the amino acid sequence (GKPGS)₄ (SEQ ID NO: 1).

In a further embodiment and in accordance with any of the above, the first and second linkers are each domain linkers.

In a further embodiment and in accordance with any of the above, the first and second Fe domains are each variant Fc domains.

In a further embodiment and in accordance with the above, the first and/or second variant Fc domains comprise one or more FcγRIIIA (CD16a) binding variant substitutions.

In a further embodiment and in accordance with the above, the one or more FcγRIIIA (CD16a) binding variant substitutions are selected from the group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with the above, the first and second variant Fc domains comprise a set of FcγRIIIA (CD16a) binding variant substitutions selected from the group including: (i) S239D/I332E: S239D/I332E, (ii) S239D: S239D, (iii) I332E: I332E, (iv) WT: S239D/I332E, (v) WT: S239D, (vi) WT: I332E, (vii) S239D/I332E: WT, (viii) S239D: WT, (ix) I332E: WT, (x) S239D/I332E: S239D, (xi) S239D/I332E: I332E, (xii) S239D: S239D/I332E, (xiii) I332E: S239D/I332E, (xiv) S239D: I332E, and (xv) I332E: S239D, wherein numbering is according to EU numbering.

In a further embodiment and in accordance with any of the above, the first and/or second variant Fc domains comprise the FcγRIIIA (CD16a) binding variant substitutions of S239D/I332E, wherein numbering is according to EU numbering.

In a further embodiment and in accordance with any of the above, the first and second variant Fc domains comprise a set of heterodimerization variants selected from the group including those depicted in FIGS. 4A-4F, wherein numbering is according to EU numbering.

In a further embodiment and in accordance with the above, the set of heterodimerization variants is selected from the group including: (i) S364K/E357Q: L368D/K370S, (ii) S364K: L368D/K370S, (iii) S364K: L368E/K370S, (iv) D401K: T411E/K360E/Q362E, and (v) T366W: T366S/L368A/Y407V, wherein numbering is according to EU numbering.

In a further embodiment and in accordance with any of the above, the first and second variant Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant Fc domain comprises one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first monomer comprises amino acid variants S364K/E357Q/E233P/L234V/L235A/G236del/S267K, wherein the second monomer comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second monomers each further comprise amino acid variants M428L/N434S, M428L/434A, or M252Y/S254T/T256E, wherein numbering is according to EU numbering.

In another aspect, a nucleic acid composition comprising nucleic acids encoding the first and second monomers and the light chain of the antibody (as described above) is provided.

In another aspect, an expression vector comprising the nucleic acids (as described above) is provided.

In another aspect, a host cell transformed with the expression vector (as described above) is provided.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, the present disclosure provides a heterodimeric antibody, comprising: (a) a first monomer comprising, from N-terminus to C-terminus, a VH1-CH1-hinge-first linker-VH1-CH1-hinge-CH2-CH3, wherein VH1 is a first variable heavy domain and CH2-CH3 is a first Fc domain; (b) a light chain comprising, from N-terminus to C-terminus, a VL1-CL, wherein VL1 is a first variable light domain and CL is a constant light domain, and wherein the VH1 and VL1 form NKp46 antigen binding domains; and (c) a second monomer comprising, from N-terminus to C-terminus, an anti-MICA/B scFv and a second Fc domain, wherein the scFv is covalently attached to the N-terminus of the second Fc domain using a second linker.

In a further embodiment and in accordance with the above, the anti-MICA/B scFv comprises a second variable heavy VH2 domain, an scFv linker, and a second variable light VL2 domain.

In a further embodiment and in accordance with any of the above, the anti-MICA/B scFv comprises a set of vhCDR1-3 and vlCDR1-3 from a variable heavy domain and variable light domain pair selected from the group including: (i) SEQ ID NOs: 240-242 for vhCDR1-3 and SEQ ID NOs: 244-246 for vlCDR1-3 of D94837_E11_1 [MICA/B]_H0_D94837_1E11_1 [MICA/B]_L0, (ii) SEQ ID NOs: 248-250 for vhCDR1-3 and SEQ ID NOs: 252-254 for vlCDR1-3 of D94837_E11_1 [MICA/B]_H1_D94837_1E11_1 [MICA/B]_L1, (iii) SEQ ID NOs: 256-258 for vhCDR1-3 and SEQ ID NOs: 260-262 for vlCDR1-3 of D94837_1E11 [MICA/B]_H1_D94837_E11_1 [MICA/B]_L2, (iv) SEQ ID NOs: 264-266 for vhCDR1-3 and SEQ ID NOs: 268-270 for vlCDR1-3 of D94837_E11_1 [MICA/B]_H2_D94837_1E11_1 [MICA/B]_L1, (v) SEQ ID NOs: 272-274 for vhCDR1-3 and SEQ ID NOs: 276-278 for vlCDR1-3 of D94837_E11_1 [MICA/B]_H2_D94837_1E11_1 [MICA/B]_L2, (vi) SEQ ID NOs: 280-282 for vhCDR1-3 and SEQ ID NOs: 284-286 for vlCDR1-3 of 2E5 [MICA/B]_H0_2E5 [MICA/B]_L0, (vii) SEQ ID NOs: 288-290 for vhCDR1-3 and SEQ ID NOs: 292-294 for vlCDR1-3 of 2E5 [MICA/B]_H1_2E5 [MICA/B]_L1, (viii) SEQ ID NOs: 296-298 for vhCDR1-3 and SEQ ID NOs: 300-302 for vlCDR1-3 of 2E5 [MICA/B]_H1_2E5 [MICA/B]_L2, (ix) SEQ ID NOs: 304-306 for vhCDR1-3 and SEQ ID NOs: 308-310 for vlCDR1-3 of 2E5 [MICA/B]_H2_2E5 [MICA/B]_L1, (x) SEQ ID NOs: 312-314 for vhCDR1-3 and SEQ ID NOs: 316-318 for vlCDR1-3 of 2E5 [MICA/B]_H2_2E5 [MICA/B]_L2, (xi) SEQ ID NOs: 320-322 for vhCDR1-3 and SEQ ID NOs: 324-326 for vlCDR1-3 of D94852_2E12 [MICA/B]_H0_D94852_2E12 [MICA/B]_L0, (xii) SEQ ID NOs: 328-330 for vhCDR1-3 and SEQ ID NOs: 332-334 for v1CDR1-3 of D94852_2E12 [MICA/B]_H1_D94852_2E12 [MICA/B]_L1, (xiii) SEQ ID NOs: 336-338 for vhCDR1-3 and SEQ ID NOs: 340-342 for v1CDR1-3 of D94852_2E12 [MICA/B]_H1_D94852_2E12 [MICA/B]_L2, (xiv) SEQ ID NOs: 344-346 for vhCDR1-3 and SEQ ID NOs: 348-350 for v1CDR1-3 of D94852_2E12 [MICA/B]_H2_D94852_2E12 [MICA/B]_L1, (xv) SEQ ID NOs: 352-354 for vhCDR1-3 and SEQ ID NOs: 356-358 for v1CDR1-3 of D94852_2E12 [MICA/B]_H2_D94852_2E12 [MICA/B]_L2, (xvi) SEQ ID NOs: 360-362 for vhCDR1-3 and SEQ ID NOs: 364-366 for v1CDR1-3 of D99136_2F7 [MICA/B]_H0_D99136_2F7 [MICA/B]_L0, (xvii) SEQ ID NOs: 368-370 for vhCDR1-3 and SEQ ID NOs: 372-374 for v1CDR1-3 of D99136_2F7 [MICA/B]_H1_D99136_2F7 [MICA/B]_L1, (xviii) SEQ ID NOs: 376-378 for vhCDR1-3 and SEQ ID NOs: 380-382 for v1CDR1-3 of D99136_2F7 [MICA/B]_H1_D99136_2F7 [MICA/B]_L2, (xix) SEQ ID NOs: 384-386 for vhCDR1-3 and SEQ ID NOs: 388-390 for v1CDR1-3 of D99136_2F7 [MICA/B]_H2_D99136_2F7 [MICA/B]_L1, (xx) SEQ ID NOs: 392-394 for vhCDR1-3 and SEQ ID NOs: 396-398 for v1CDR1-3 of D99136_2F7 [MICA/B]_H2_D99136_2F7 [MICA/B]_L2, (xxi) SEQ ID NOs: 400-402 for vhCDR1-3 and SEQ ID NOs: 404-406 for v1CDR1-3 of D103388_1C7 [MICA/B]_H0_D103388_1C7 [MICA/B]_L0, (xxii) SEQ ID NOs: 408-410 for vhCDR1-3 and SEQ ID NOs: 412-414 for v1CDR1-3 of D103388_1C7 [MICA/B]_H1_D103388_1C7 [MICA/B]_L1, (xxiii) SEQ ID NOs: 416-418 for vhCDR1-3 and SEQ ID NOs: 420-422 for v1CDR1-3 of D103388_1C7 [MICA/B]_H1_D103388_1C7 [MICA/B]_L2, (xxiv) SEQ ID NOs: 424-426 for vhCDR1-3 and SEQ ID NOs: 428-430 for v1CDR1-3 of D103388_1C7 [MICA/B]_H2_D103388_1C7 [MICA/B]_L1, (xxv) SEQ ID NOs: 432-434 for vhCDR1-3 and SEQ ID NOs: 436-438 for v1CDR1-3 of D103388_1C7 [MICA/B]_H2_D103388_1C7 [MICA/B]_L2, (xxvi) SEQ ID NOs: 440-442 for vhCDR1-3 and SEQ ID NOs: 444-446 for v1CDR1-3 of D103388_1D7 [MICA/B]_H0_D103388_1D7 [MICA/B]_L0, (xxvii) SEQ ID NOs: 448-450 for vhCDR1-3 and SEQ ID NOs: 452-454 for v1CDR1-3 of D103388_1D7 [MICA/B]_H1_D103388_1D7 [MICA/B]_L1, (xxviii) SEQ ID NOs: 456-458 for vhCDR1-3 and SEQ ID NOs: 460-462 for v1CDR1-3 of D103388_1D7 [MICA/B]_H1_D103388_1D7 [MICA/B]_L2, (xxix) SEQ ID NOs: 464-466 for vhCDR1-3 and SEQ ID NOs: 468-470 for v1CDR1-3 of D103388_1D7 [MICA/B]_H2_D103388_1D7 [MICA/B]_L1, (xxx) SEQ ID NOs: 472-474 for vhCDR1-3 and SEQ ID NOs: 476-478 for v1CDR1-3 of D103388_1D7 [MICA/B]_H2_D103388_1D7 [MICA/B]_L2, (xxxi) SEQ ID NOs: 480-482 for vhCDR1-3 and SEQ ID NOs: 484-486 for v1CDR1-3 of D105317_1A2 [MICA/B]_H0_D105317_1A2 [MICA/B]_L0, (xxxii) SEQ ID NOs: 488-490 for vhCDR1-3 and SEQ ID NOs: 492-494 for v1CDR1-3 of D105317_1A2 [MICA/B]_H1_D105317_1A2 [MICA/B]_L1, (xxxiii) SEQ ID NOs: 496-498 for vhCDR1-3 and SEQ ID NOs: 500-502 for v1CDR1-3 of D105317_1A2 [MICA/B]_H1_D105317_1A2 [MICA/B]_L2, (xxxiv) SEQ ID NOs: 504-506 for vhCDR1-3 and SEQ ID NOs: 508-510 for v1CDR1-3 of D105317_1A2 [MICA/B]_H2_D105317_1A2 [MICA/B]_L1, (xxxv) SEQ ID NOs: 512-514 for vhCDR1-3 and SEQ ID NOs: 516-518 for vlCDR1-3 of D105317_1A2 [MICA/B]_H2_D105317_1A2 [MICA/B]_L2, (xxxvi) SEQ ID NOs: 520-522 for vhCDR1-3 and SEQ ID NOs: 524-526 for vlCDR1-3 of D99136_2C11 [MICA/B]_H0_D99i36_2C11 [MICA/B]_L0, (xxxvii) SEQ ID NOs: 528-530 for vhCDR1-3 and SEQ ID NOs: 532-534 for v1CDR1-3 of D99136_2C11 [MICA/B]_H1_D99136_2C11 [MICA/B]_L1, (xxxviii) SEQ ID NOs: 536-538 for vhCDR1-3 and SEQ ID NOs: 540-542 for v1CDR1-3 of D99136_2C11 [MICA/B]_H1_D99136_2C11 [MICA/B]_L2, (xxxix) SEQ ID NOs: 544-546 for vhCDR1-3 and SEQ ID NOs: 548-550 for v1CDR1-3 of D99136_2C11 [MICA/B]_H2_D99136_2C11 [MICA/B]_L1, (xl) SEQ ID NOs: 552-554 for vhCDR1-3 and SEQ ID NOs: 556-558 for v1CDR1-3 of D99136_2C11 [MICA/B]_H2_D99136_2C11 [MICA/B]_L2, (xli) SEQ ID NOs: 560-562 for vhCDR1-3 and SEQ ID NOs: 564-566 for v1CDR1-3 of D103388_1B11-2 [MICA/B]_H0_D103388_1B11-2 [MICA/B]_L0, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (lvii) SEQ ID NOs: 680-682 for vhCDR1-3 and SEQ ID NOs: 684-686 for vlCDR1-3 of 3F9[MICA/B]_H0_3F9[MICA/B]_L0, (lviii) SEQ ID NOs: 688-690 for vhCDR1-3 and SEQ ID NOs: 692-694 for vlCDR1-3 of 6E1[MICA/B]_H0_6E1[MICA/B]_L0, (lix) SEQ ID NOs: 696-698 for vhCDR1-3 and SEQ ID NOs: 700-702 for vlCDR1-3 of 7C6[MICA/B]_H0_7C6[MICA/B]_L0, (lx) SEQ ID NOs: 704-706 for vhCDR1-3 and SEQ ID NOs: 708-710 for vlCDR1-3 of 13A9 [MICA/B]_H0_13A9 [MICA/B]_L0, and (lxi) SEQ ID NOs: 712-714 for vhCDR1-3 and SEQ ID NOs: 716-718 for vlCDR1-3 of 1D5 [MICA/B]_H0_1D5 [MICA/B]_L0, as depicted in FIGS. 21 and 22.

In a further embodiment and in accordance with any of the above, the anti-MICA/B scFv comprises a variable heavy domain and variable light domain pair selected from the group including: (i) SEQ ID Nos: 239 and 243 for D94837_1E11_1 [MICA/B]_H0_D94837_1E11_1 [MICA/B]_L0, (ii) SEQ ID Nos: 247 and 251 for D94837_1E11_1 [MICA/B]_H1_D94837_1E11_1 [MICA/B]_L1, (iii) SEQ ID Nos: 255 and 259 for D94837_1E11_1 [MICA/B]_H1_D94837_1E1_1 [MICA/B]_L2, (iv) SEQ ID Nos: 263 and 267 for D94837_1E11_1 [MICA/B]_H2_D94837_1E11_1 [MICA/B]_L1, (v) SEQ ID Nos: 271 and 275 for D94837_1E11_1 [MICA/B]_H2_D94837_1E11_1 [MICA/B]_L2, (vi) SEQ ID Nos: 279 and 283 for 2E5 [MICA/B]_H0_2E5 [MICA/B]_L0, (vii) SEQ ID Nos: 287 and 291 for 2E5 [MICA/B]_H1_2E5 [MICA/B]_L1, (viii) SEQ ID Nos: 295 and 299 for 2E5 [MICA/B]_H1_2E5 [MICA/B]_L2, (ix) SEQ ID Nos: 303 and 307 for 2E5 [MICA/B]_H2_2E5 [MICA/B]_L1, (x) SEQ ID Nos: 311 and 315 for 2E5 [MICA/B]_H2_2E5 [MICA/B]_L2, (xi) SEQ ID Nos: 319 and 323 for D94852_2E12 [MICA/B]_H0_D94852_2E12 [MICA/B]_L0, (xii) SEQ ID Nos: 327 and 331 for D94852_2E12 [MICA/B]_H1_D94852_2E12 [MICA/B]_L1, (xiii) SEQ ID Nos: 335 and 339 for D94852_2E12 [MICA/B]_H1_D94852_2E12 [MICA/B]_L2, (xiv) SEQ ID Nos: 343 and 347 for D94852_2E12 [MICA/B]_H2_D94852_2E12 [MICA/B]_L1, (xv) SEQ ID Nos: 351 and 355 for D94852_2E12 [MICA/B]_H2_D94852_2E12 [MICA/B]_L2, (xvi) SEQ ID Nos: 359 and 363 for D99136_2F7 [MICA/B]_H0_D99136_2F7 [MICA/B]_L0, (xvii) SEQ ID Nos: 367 and 371 for D99136_2F7 [MICA/B]_H1_D99136_2F7 [MICA/B]_L1, (xviii) SEQ ID Nos: 375 and 379 for D99136_2F7 [MICA/B]_H1_D99136_2F7 [MICA/B]_L2, (xix) SEQ ID Nos: 383 and 387 for D99136_2F7 [MICA/B]_H2_D99136_2F7 [MICA/B]_L1, (xx) SEQ ID Nos: 391 and 395 for D99136_2F7 [MICA/B]_H2_D99136_2F7 [MICA/B]_L2, (xxi) SEQ ID Nos: 399 and 403 for D103388_1C7 [MICA/B]_H0_D103388_1C7 [MICA/B]_L0, (xxii) SEQ ID Nos: 407 and 411 for D103388_1C7 [MICA/B]_H1_D103388_1C7 [MICA/B]_L1, (xxiii) SEQ ID Nos: 415 and 419 for D103388_1C7 [MICA/B]_H1_D103388_1C7 [MICA/B]_L2, (xxiv) SEQ ID Nos: 423 and 427 for D103388_1C7 [MICA/B]_H2_D103388_1C7 [MICA/B]_L1, (xxv) SEQ ID Nos: 431 and 435 for D103388_1C7 [MICA/B]_H2_D103388_1C7 [MICA/B]_L2, (xxvi) SEQ ID Nos: 439 and 443 for D103388_1D7 [MICA/B]_H0_D103388_1D7 [MICA/B]_L0, (xxvii) SEQ ID Nos: 447 and 451 for D103388_1D7 [MICA/B]_H1_D103388_1D7 [MICA/B]_L1, (xxviii) SEQ ID Nos: 455 and 459 for D103388_1D7 [MICA/B]_H1_D103388_1D7 [MICA/B]_L2, (xxix) SEQ ID Nos: 463 and 467 for D103388_1D7 [MICA/B]_H2_D103388_1D7 [MICA/B]_L1, (xxx) SEQ ID Nos: 471 and 475 for D103388_1D7 [MICA/B]_H2_D103388_1D7 [MICA/B]_L2, (xxxi) SEQ ID Nos: 479 and 483 for D105317_1A2 [MICA/B]_H0_D105317_1A2 [MICA/B]_L0, (xxxii) SEQ ID Nos: 487 and 491 for D105317_1A2 [MICA/B]_H1_D105317_1A2 [MICA/B]_L1, (xxxiii) SEQ ID Nos: 495 and 499 for D105317_1A2 [MICA/B]_H1_D105317_1A2 [MICA/B]_L2, (xxxiv) SEQ ID Nos: 503 and 507 for D105317_1A2 [MICA/B]_H2_D105317_1A2 [MICA/B]_L1, (xxxv) SEQ ID Nos: 511 and 515 for D105317_1A2 [MICA/B]_H2_D105317_1A2 [MICA/B]_L2, (xxxvi) SEQ ID Nos: 519 and 523 for D99136_2C11 [MICA/B]_H0_D99136_2C11 [MICA/B]_L0, (xxxvii) SEQ ID Nos: 527 and 531 for D99136_2C11 [MICA/B]_H1_D99136_2C11 [MICA/B]_L1, (xxxviii) SEQ ID Nos: 535 and 539 for D99136_2C11 [MICA/B]_H1_D99136_2C11 [MICA/B]_L2, (xxxix) SEQ ID Nos: 543 and 547 for D99136_2C11 [MICA/B]_H2_D99136_2C11 [MICA/B]_L1, (xl) SEQ ID Nos: 551 and 555 for D99136_2C11 [MICA/B]_H2_D99136_2C11 [MICA/B]_L2, (xli) SEQ ID Nos: 559 and 563 for D103388_1B11-2 [MICA/B]_H0_D103388_1B11-2 [MICA/B]_L0, (xlii) SEQ ID Nos: 567 and 571 for D105317_1B6 [MICA/B]_H0_D105317_1B6 [MICA/B]_L0, (xliii) SEQ ID Nos: 575 and 579 for D105317_1C8 [MICA/B]_H0_D105317_1C8 [MICA/B]_L0, (xliv) SEQ ID Nos: 583 and 587 for D105317_1F6 [MICA/B]_H0_D105317_1F6 [MICA/B]_L0, (xlv) SEQ ID Nos: 591 and 595 for D105317_1F7 [MICA/B]_H0_D105317_1F7 [MICA/B]_L0, (xlvi) SEQ ID Nos: 599 and 603 for D94837_1D3 [MICA/B]_H0_D94837_1D3 [MICA/B]_L0, (xlvii) SEQ ID Nos: 607 and 611 for D94837_1D8 [MICA/B]_H0_D94837_1D8 [MICA/B]_L0, (xlviii) SEQ ID Nos: 615 and 619 for D94837_1D9 [MICA/B]_H0_D94837_1D9 [MICA/B]_L0, (xlix) SEQ ID Nos: 623 and 627 for D94837_1E1 [MICA/B]_H0_D94837_1E1 [MICA/B]_L0, (1) SEQ ID Nos: 631 and 635 for D94852_2D4 [MICA/B]_H0_D94852_2D4 [MICA/B]_L0, (li) SEQ ID Nos: 639 and 643 for D94852_2G8 [MICA/B]_H0_D94852_2G8 [MICA/B]_L0, (lii) SEQ ID Nos: 647 and 651 for D88487_2A8 [MICA/B]_H0_D88487_2A8 [MICA/B]_L0, (liii) SEQ ID Nos: 655 and 659 for D94837_3B12 [MICA/B]_H0_D94837_3B12 [MICA/B]_L0, (liv) SEQ ID Nos: 663 and 667 for D99122_1H7 [MICA/B]_H0_D99122_1H7 [MICA/B]_L0, (lv) SEQ ID Nos: 671 and 675 for D99136_2E8 [MICA/B]_H0_D99136_2E8 [MICA/B]_L0, (lvi) SEQ ID Nos: 647 and 651 for D88487_2A8 [MICA/B]_H0_D88487_2A8 [MICA/B]_L0, (lvii) SEQ ID Nos: 679 and 683 for 3F9[MICA/B]_H0_3F9[MICA/B]_L0, (lviii) SEQ ID Nos: 687 and 691 for 6E1[MICA/B]_H0_6E1[MICA/B]_L0, (lix) SEQ ID Nos: 695 and 699 for 7C6[MICA/B]_H0_7C6[MICA/B]_L0, (lx) SEQ ID Nos: 703 and 707 for 13A9 [MICA/B]_H0_13A9 [MICA/B]_L0, and (lxi) SEQ ID Nos: 711 and 715 for 1D5 [MICA/B]_H0_1D5 [MICA/B]_L0, as depicted in FIGS. 21 and 22.

In a further embodiment and in accordance with any of the above, each of the NKp46 antigen binding domains comprise a set of vhCDR1-3 and vlCDR1-3 from a variable heavy domain and variable light domain pair, wherein the set of vhCDR1-3 and vlCDR1-3 is selected from the group including: (i) SEQ ID NOs: 720-722 for vhCDR1-3 and SEQ ID NOs: 724-726 for vlCDR1-3 of 2C10A3.372[NKp46]_H1_2C10A3.372[NKp46]_L1, and (ii) SEQ ID NOs: 728-730 for vhCDR1-3 and SEQ ID NOs: 732-734 for vlCDR1-3 of NKp46-A[NKp46]_H_NKp46-A[NKp46]_L, as depicted in FIGS. 23 and 24.

In a further embodiment and in accordance with any of the above, each of the NKp46 antigen binding domains comprise a variable heavy domain and variable light domain pair selected from the group including: (i) SEQ ID Nos: 719 and 723 for 2C10A3.372[NKp46]_H1_2C10A3.372[NKp46]_L1, and (ii) SEQ ID Nos: 727 and 731 for NKp46-A[NKp46]_H_NKp46-A[NKp46]_L, as depicted in FIGS. 23 and 24.

In a further embodiment and in accordance with any of the above, the scFv linker is a charged scFv linker.

In a further embodiment and in accordance with the above, the scFv linker is a charged scFv linker having the amino acid sequence (GKPGS)₄ (SEQ ID NO: 1).

In a further embodiment and in accordance with any of the above, the first and second linkers are each domain linkers.

In a further embodiment and in accordance with any of the above, the first and second Fc domains are each variant Fc domains.

In a further embodiment and in accordance with the above, the first and/or second variant Fc domains comprise one or more FcγRIIIA (CD16a) binding variant substitutions.

In a further embodiment and in accordance with the above, the one or more FcγRIIIA (CD16a) binding variant substitutions are selected from the group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with the above, the first and second variant Fc domains comprise a set of FcγRIIIA (CD16a) binding variant substitutions selected from the group including: (i) S239D/I332E: S239D/I332E, (ii) S239D: S239D, (iii) 1332E: 1332E, (iv) WT: S239D/I332E, (v) WT: S239D, (vi) WT: 1332E, (vii) S239D/I332E: WT, (viii) S239D: WT, (ix) 1332E: WT, (x) S239D/I332E: S239D, (xi) S239D/I332E: 1332E, (xii) S239D: S239D/I332E, (xiii) 1332E: S239D/I332E, (xiv) S239D: 1332E, and (xv) 1332E: S239D, wherein numbering is according to EU numbering.

In a further embodiment and in accordance with any of the above, the first and/or second variant Fc domains comprise the FcγRIIIA (CD16a) binding variant substitutions of S239D/I332E, wherein numbering is according to EU numbering.

In a further embodiment and in accordance with any of the above, the first and second variant Fc domains comprise a set of heterodimerization variants selected from the group including those depicted in FIGS. 4A-4F, wherein numbering is according to EU numbering.

In a further embodiment and in accordance with the above, the set of heterodimerization variants is selected from the group including: (i) S364K/E357Q: L368D/K370S, (ii) S364K: L368D/K370S, (iii) S364K: L368E/K370S, (iv) D401K: T411E/K360E/Q362E, and (v) T366W: T366S/L368A/Y407V, wherein numbering is according to EU numbering.

In a further embodiment and in accordance with any of the above, the first and second variant Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant Fc domain comprises one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first monomer comprises amino acid variants S364K/E357Q/E233P/L234V/L235A/G236del/S267K, wherein the second monomer comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second monomers each further comprise amino acid variants M428L/N434S, M428L/434A, or M252Y/S254T/T256E, wherein numbering is according to EU numbering.

In another aspect, a nucleic acid composition comprising nucleic acids encoding the first and second monomers and the light chain of the antibody (as described above) is provided.

In another aspect, an expression vector comprising the nucleic acids (as described above) is provided.

In another aspect, a host cell transformed with the expression vector (as described above) is provided.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, the present disclosure provides a heterodimeric antibody, comprising: (a) a first monomer comprising, from N-terminus to C-terminus, a VH1-CH1-hinge-CH2-CH3-domain linker-scFv, wherein VH1 is a first variable heavy domain, scFv is an anti-NKp46 scFv, and CH2-CH3 is a first Fc domain; (b) a second monomer comprising, from N-terminus to C-terminus, a VH2-CH1-hinge-CH2-CH3, wherein CH2-CH3 is a second Fc domain; and (c) a light chain comprising, from N-terminus to C-terminus, a VL1-CL, wherein VL1 is a first variable light domain and CL is a constant light domain, wherein: (i) the VH1 and the VL1 form a first MICA/B antigen binding domain, (ii) the VH2 domain and the VL1 form a second MICA/B antigen binding domain, and (iii) the anti-NKp46 scFv comprises a VH3 domain, a scFv linker, and a VL2 domain.

In a further embodiment and in accordance with the above, the first and/or second MICA/B antigen binding domains comprise a set of vhCDR1-3 and vlCDR1-3 from a variable heavy domain and variable light domain pair selected from the group including: (i) SEQ ID NOs: 240-242 for vhCDR1-3 and SEQ ID NOs: 244-246 for vlCDR1-3 of D94837_1E1_1 [MICA/B]_H0_D94837_E11_1 [MICA/B]_L0, (ii) SEQ ID NOs: 248-250 for vhCDR1-3 and SEQ ID NOs: 252-254 for vlCDR1-3 of D94837_E11_1 [MICA/B]_H1_D94837_1E11_1 [MICA/B]_L1, (iii) SEQ ID NOs: 256-258 for vhCDR1-3 and SEQ ID NOs: 260-262 for v1CDR1-3 of D94837_E11_1 [MICA/B]_H1_D94837_1E11_1 [MICA/B]_L2, (iv) SEQ ID NOs: 264-266 for vhCDR1-3 and SEQ ID NOs: 268-270 for v1CDR1-3 of D94837_1E11_1 [MICA/B]_H2_D94837_1E11_1 [MICA/B]_L1, (v) SEQ ID NOs: 272-274 for vhCDR1-3 and SEQ ID NOs: 276-278 for v1CDR1-3 of D94837_1E11_1 [MICA/B]_H2_D94837_1E11_1 [MICA/B]_L2, (vi) SEQ ID NOs: 280-282 for vhCDR1-3 and SEQ ID NOs: 284-286 for v1CDR1-3 of 2E5 [MICA/B]_H0_2E5 [MICA/B]_L0, (vii) SEQ ID NOs: 288-290 for vhCDR1-3 and SEQ ID NOs: 292-294 for v1CDR1-3 of 2E5 [MICA/B]_H1_2E5 [MICA/B]_L1, (viii) SEQ ID NOs: 296-298 for vhCDR1-3 and SEQ ID NOs: 300-302 for v1CDR1-3 of 2E5 [MICA/B]_H1_2E5 [MICA/B]_L2, (ix) SEQ ID NOs: 304-306 for vhCDR1-3 and SEQ ID NOs: 308-310 for v1CDR1-3 of 2E5 [MICA/B]_H2_2E5 [MICA/B]_L1, (x) SEQ ID NOs: 312-314 for vhCDR1-3 and SEQ ID NOs: 316-318 for v1CDR1-3 of 2E5 [MICA/B]_H2_2E5 [MICA/B]_L2, (xi) SEQ ID NOs: 320-322 for vhCDR1-3 and SEQ ID NOs: 324-326 for v1CDR1-3 of D94852_2E12 [MICA/B]_H0_D94852_2E12 [MICA/B]_L0, (xii) SEQ ID NOs: 328-330 for vhCDR1-3 and SEQ ID NOs: 332-334 for v1CDR1-3 of D94852_2E12 [MICA/B]_H1_D94852_2E12 [MICA/B]_L1, (xiii) SEQ ID NOs: 336-338 for vhCDR1-3 and SEQ ID NOs: 340-342 for v1CDR1-3 of D94852_2E12 [MICA/B]_H1_D94852_2E12 [MICA/B]_L2, (xiv) SEQ ID NOs: 344-346 for vhCDR1-3 and SEQ ID NOs: 348-350 for v1CDR1-3 of D94852_2E12 [MICA/B]_H2_D94852_2E12 [MICA/B]_L1, (xv) SEQ ID NOs: 352-354 for vhCDR1-3 and SEQ ID NOs: 356-358 for v1CDR1-3 of D94852_2E12 [MICA/B]_H2_D94852_2E12 [MICA/B]_L2, (xvi) SEQ ID NOs: 360-362 for vhCDR1-3 and SEQ ID NOs: 364-366 for v1CDR1-3 of D99136_2F7 [MICA/B]_H0_D99136_2F7 [MICA/B]_L0, (xvii) SEQ ID NOs: 368-370 for vhCDR1-3 and SEQ ID NOs: 372-374 for v1CDR1-3 of D99136_2F7 [MICA/B]_H1_D99136_2F7 [MICA/B]_L1, (xviii) SEQ ID NOs: 376-378 for vhCDR1-3 and SEQ ID NOs: 380-382 for v1CDR1-3 of D99136_2F7 [MICA/B]_H1_D99136_2F7 [MICA/B]_L2, (xix) SEQ ID NOs: 384-386 for vhCDR1-3 and SEQ ID NOs: 388-390 for v1CDR1-3 of D99136_2F7 [MICA/B]_H2_D99136_2F7 [MICA/B]_L1, (xx) SEQ ID NOs: 392-394 for vhCDR1-3 and SEQ ID NOs: 396-398 for v1CDR1-3 of D99136_2F7 [MICA/B]_H2_D99136_2F7 [MICA/B]_L2, (xxi) SEQ ID NOs: 400-402 for vhCDR1-3 and SEQ ID NOs: 404-406 for v1CDR1-3 of D103388_1C7 [MICA/B]_H0_D103388_1C7 [MICA/B]_L0, (xxii) SEQ ID NOs: 408-410 for vhCDR1-3 and SEQ ID NOs: 412-414 for v1CDR1-3 of D103388_1C7 [MICA/B]_H1_D103388_1C7 [MICA/B]_L1, (xxiii) SEQ ID NOs: 416-418 for vhCDR1-3 and SEQ ID NOs: 420-422 for v1CDR1-3 of D103388_1C7 [MICA/B]_H1_D103388_1C7 [MICA/B]_L2, (xxiv) SEQ ID NOs: 424-426 for vhCDR1-3 and SEQ ID NOs: 428-430 for v1CDR1-3 of D103388_1C7 [MICA/B]_H2_D103388_1C7 [MICA/B]_L1, (xxv) SEQ ID NOs: 432-434 for vhCDR1-3 and SEQ ID NOs: 436-438 for v1CDR1-3 of D103388_1C7 [MICA/B]_H2_D103388_1C7 [MICA/B]_L2, (xxvi) SEQ ID NOs: 440-442 for vhCDR1-3 and SEQ ID NOs: 444-446 for v1CDR1-3 of D103388_1D7 [MICA/B]_H0_D103388_1D7 [MICA/B]_L0, (xxvii) SEQ ID NOs: 448-450 for vhCDR1-3 and SEQ ID NOs: 452-454 for v1CDR1-3 of D103388_1D7 [MICA/B]_H1_D103388_1D7 [MICA/B]_L1, (xxviii) SEQ ID NOs: 456-458 for vhCDR1-3 and SEQ ID NOs: 460-462 for v1CDR1-3 of D103388_1D7 [MICA/B]_H1_D103388_1D7 [MICA/B]_L2, (xxix) SEQ ID NOs: 464-466 for vhCDR1-3 and SEQ ID NOs: 468-470 for v1CDR1-3 of D103388_1D7 [MICA/B]_H2_D103388_1D7 [MICA/B]_L1, (xxx) SEQ ID NOs: 472-474 for vhCDR1-3 and SEQ ID NOs: 476-478 for v1CDR1-3 of D103388_1D7 [MICA/B]_H2_D103388_1D7 [MICA/B]_L2, (xxxi) SEQ ID NOs: 480-482 for vhCDR1-3 and SEQ ID NOs: 484-486 for v1CDR1-3 of D105317_1A2 [MICA/B]_H0_D105317_1A2 [MICA/B]_L0, (xxxii) SEQ ID NOs: 488-490 for vhCDR1-3 and SEQ ID NOs: 492-494 for v1CDR1-3 of D105317_1A2 [MICA/B]_H1_D105317_1A2 [MICA/B]_L1, (xxxiii) SEQ ID NOs: 496-498 for vhCDR1-3 and SEQ ID NOs: 500-502 for v1CDR1-3 of D105317_1A2 [MICA/B]_H1_D105317_1A2 [MICA/B]_L2, (xxxiv) SEQ ID NOs: 504-506 for vhCDR1-3 and SEQ ID NOs: 508-510 for vlCDR1-3 of D105317_1A2 [MICA/B]_H2_D105317_1A2 [MICA/B]_L1, (xxxv) SEQ ID NOs: 512-514 for vhCDR1-3 and SEQ ID NOs: 516-518 for v1CDR1-3 of D105317_1A2 [MICA/B]_H2_D105317_1A2 [MICA/B]_L2, (xxxvi) SEQ ID NOs: 520-522 for vhCDR1-3 and SEQ ID NOs: 524-526 for v1CDR1-3 of D99136_2C11 [MICA/B]_H0_D99136_2C11 [MICA/B]_L0, (xxxvii) SEQ ID NOs: 528-530 for vhCDR1-3 and SEQ ID NOs: 532-534 for vlCDR1-3 of D99136_2C11 [MICA/B]_H1_D99i36_2C11 [MICA/B]_L1, (xxxviii) SEQ ID NOs: 536-538 for vhCDR1-3 and SEQ ID NOs: 540-542 for v1CDR1-3 of D99136_2C11 [MICA/B]_H1_D99136_2C11 [MICA/B]_L2, (xxxix) SEQ ID NOs: 544-546 for vhCDR1-3 and SEQ ID NOs: 548-550 for v1CDR1-3 of D99136_2C11 [MICA/B]_H2_D99136_2C11 [MICA/B]_L1, (xl) SEQ ID NOs: 552-554 for vhCDR1-3 and SEQ ID NOs: 556-558 for vlCDR1-3 of D99136_2C11 [MICA/B]_H2_D99136_2C11 [MICA/B]_L2, (xli) SEQ ID NOs: 560-562 for vhCDR1-3 and SEQ ID NOs: 564-566 for vlCDR1-3 of D103388_1B11-2 [MICA/B]_H0_D103388_1B11-2 [MICA/B]_L0, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (lvii) SEQ ID NOs: 680-682 for vhCDR1-3 and SEQ ID NOs: 684-686 for vlCDR1-3 of 3F9[MICA/B]_H0_3F9[MICA/B]_L0, (lviii) SEQ ID NOs: 688-690 for vhCDR1-3 and SEQ ID NOs: 692-694 for vlCDR1-3 of 6E1[MICA/B]_H0_6E1[MICA/B]_L0, (lix) SEQ ID NOs: 696-698 for vhCDR1-3 and SEQ ID NOs: 700-702 for vlCDR1-3 of 7C6[MICA/B]_H0_7C6[MICA/B]_L0, (lx) SEQ ID NOs: 704-706 for vhCDR1-3 and SEQ ID NOs: 708-710 for vlCDR1-3 of 13A9 [MICA/B]_H0_13A9 [MICA/B]_L0, and (lxi) SEQ ID NOs: 712-714 for vhCDR1-3 and SEQ ID NOs: 716-718 for vlCDR1-3 of 1D5 [MICA/B]_H0_1D5 [MICA/B]_L0, as depicted in FIGS. 21 and 22.

In a further embodiment and in accordance with any of the above, the first and/or second MICA/B antigen binding domains comprise a variable heavy domain and variable light domain pair selected from the group including: (i) SEQ ID Nos: 239 and 243 for D94837_1E11_1 [MICA/B]_H0_D94837_1E11_1 [MICA/B]_L0, (ii) SEQ ID Nos: 247 and 251 for D94837_1E1_1 [MICA/B]_H1_D94837_E11_1 [MICA/B]_L1, (iii) SEQ ID Nos: 255 and 259 for D94837_1E11_1 [MICA/B]_H1_D94837_1E11_1 [MICA/B]_L2, (iv) SEQ ID Nos: 263 and 267 for D94837_E11_1 [MICA/B]_H2_D94837_E11_1 [MICA/B]_L1, (v) SEQ ID Nos: 271 and 275 for D94837_E11_1 [MICA/B]_H2_D94837_E11_1 [MICA/B]_L2, (vi) SEQ ID Nos: 279 and 283 for 2E5 [MICA/B]_H0_2E5 [MICA/B]_L0, (vii) SEQ ID Nos: 287 and 291 for 2E5 [MICA/B]_H1_2E5 [MICA/B]_L1, (viii) SEQ ID Nos: 295 and 299 for 2E5 [MICA/B]_H1_2E5 [MICA/B]_L2, (ix) SEQ ID Nos: 303 and 307 for 2E5 [MICA/B]_H2_2E5 [MICA/B]_L1, (x) SEQ ID Nos: 311 and 315 for 2E5 [MICA/B]_H2_2E5 [MICA/B]_L2, (xi) SEQ ID Nos: 319 and 323 for D94852_2E12 [MICA/B]_H0_D94852_2E12 [MICA/B]_L0, (xii) SEQ ID Nos: 327 and 331 for D94852_2E12 [MICA/B]_H1_D94852_2E12 [MICA/B]_L1, (xiii) SEQ ID Nos: 335 and 339 for D94852_2E12 [MICA/B]_H1_D94852_2E12 [MICA/B]_L2, (xiv) SEQ ID Nos: 343 and 347 for D94852_2E12 [MICA/B]_H2_D94852_2E12 [MICA/B]_L1, (xv) SEQ ID Nos: 351 and 355 for D94852_2E12 [MICA/B]_H2_D94852_2E12 [MICA/B]_L2, (xvi) SEQ ID Nos: 359 and 363 for D99136_2F7 [MICA/B]_H0_D99136_2F7 [MICA/B]_L0, (xvii) SEQ ID Nos: 367 and 371 for D99136_2F7 [MICA/B]_H1_D99136_2F7 [MICA/B]_L1, (xviii) SEQ ID Nos: 375 and 379 for D99136_2F7 [MICA/B]_H1_D99136_2F7 [MICA/B]_L2, (xix) SEQ ID Nos: 383 and 387 for D99136_2F7 [MICA/B]_H2_D99136_2F7 [MICA/B]_L1, (xx) SEQ ID Nos: 391 and 395 for D99136_2F7 [MICA/B]_H2_D99136_2F7 [MICA/B]_L2, (xxi) SEQ ID Nos: 399 and 403 for D103388_1C7 [MICA/B]_H0_D103388_1C7 [MICA/B]_L0, (xxii) SEQ ID Nos: 407 and 411 for D103388_1C7 [MICA/B]_H1_D103388_1C7 [MICA/B]_L1, (xxiii) SEQ ID Nos: 415 and 419 for D103388_1C7 [MICA/B]_H1_D103388_1C7 [MICA/B]_L2, (xxiv) SEQ ID Nos: 423 and 427 for D103388_1C7 [MICA/B]_H2_D103388_1C7 [MICA/B]_L1, (xxv) SEQ ID Nos: 431 and 435 for D103388_1C7 [MICA/B]_H2_D103388_1C7 [MICA/B]_L2, (xxvi) SEQ ID Nos: 439 and 443 for D103388_1D7 [MICA/B]_H0_D103388_1D7 [MICA/B]_L0, (xxvii) SEQ ID Nos: 447 and 451 for D103388_1D7 [MICA/B]_H1_D103388_1D7 [MICA/B]_L1, (xxviii) SEQ ID Nos: 455 and 459 for D103388_1D7 [MICA/B]_H1D103388_1D7 [MICA/B]_L2, (xxix) SEQ ID Nos: 463 and 467 for D103388_1D7 [MICA/B]_H2_D103388_1D7 [MICA/B]_L1, (xxx) SEQ ID Nos: 471 and 475 for D103388_1D7 [MICA/B]_H2_D103388_1D7 [MICA/B]_L2, (xxxi) SEQ ID Nos: 479 and 483 for D105317_1A2 [MICA/B]_H0_D105317_1A2 [MICA/B]_L0, (xxxii) SEQ ID Nos: 487 and 491 for D105317_1A2 [MICA/B]_H1_D105317_1A2 [MICA/B]_L1, (xxxiii) SEQ ID Nos: 495 and 499 for D105317_1A2 [MICA/B]_H1_D105317_1A2 [MICA/B]_L2, (xxxiv) SEQ ID Nos: 503 and 507 for D105317_1A2 [MICA/B]_H2_D105317_1A2 [MICA/B]_L1, (xxxv) SEQ ID Nos: 511 and 515 for D105317_1A2 [MICA/B]_H2_D105317_1A2 [MICA/B]_L2, (xxxvi) SEQ ID Nos: 519 and 523 for D99136_2C11 [MICA/B]_H0_D99136_2C11 [MICA/B]_L0, (xxxvii) SEQ ID Nos: 527 and 531 for D99136_2C11 [MICA/B]_H1_D99136_2C11 [MICA/B]_L1, (xxxviii) SEQ ID Nos: 535 and 539 for D99136_2C11 [MICA/B]_H1_D99136_2C11 [MICA/B]_L2, (xxxix) SEQ ID Nos: 543 and 547 for D99136_2C11 [MICA/B]_H2_D99136_2C11 [MICA/B]_L1, (xl) SEQ ID Nos: 551 and 555 for D99136_2C11 [MICA/B]_H2_D99136_2C11 [MICA/B]_L2, (xli) SEQ ID Nos: 559 and 563 for D103388_1B11-2 [MICA/B]_H0_D103388_1B11-2 [MICA/B]_L0, (xlii) SEQ ID Nos: 567 and 571 for D105317_1B6 [MICA/B]_H0_D105317_1B6 [MICA/B]_L0, (xliii) SEQ ID Nos: 575 and 579 for D105317_1C8 [MICA/B]_H0_D105317_1C8 [MICA/B]_L0, (xliv) SEQ ID Nos: 583 and 587 for D105317_1F6 [MICA/B]_H0_D105317_1F6 [MICA/B]_L0, (xlv) SEQ ID Nos: 591 and 595 for D105317_1F7 [MICA/B]_H0_D105317_1F7 [MICA/B]_L0, (xlvi) SEQ ID Nos: 599 and 603 for D94837_1D3 [MICA/B]_H0_D94837_1D3 [MICA/B]_L0, (xlvii) SEQ ID Nos: 607 and 611 for D94837_1D8 [MICA/B]_H0_D94837_1D8 [MICA/B]_L0, (xlviii) SEQ ID Nos: 615 and 619 for D94837_1D9 [MICA/B]_H0_D94837_1D9 [MICA/B]_L0, (xlix) SEQ ID Nos: 623 and 627 for D94837_1E1 [MICA/B]_H0_D94837_1E1 [MICA/B]_L0, (1) SEQ ID Nos: 631 and 635 for D94852_2D4 [MICA/B]_H0_D94852_2D4 [MICA/B]_L0, (li) SEQ ID Nos: 639 and 643 for D94852_2G8 [MICA/B]_H0_D94852_2G8 [MICA/B]_L0, (lii) SEQ ID Nos: 647 and 651 for D88487_2A8 [MICA/B]_H0_D88487_2A8 [MICA/B]_L0, (liii) SEQ ID Nos: 655 and 659 for D94837_3B12 [MICA/B]_H0_D94837_3B12 [MICA/B]_L0, (liv) SEQ ID Nos: 663 and 667 for D99122_1H7 [MICA/B]_H0_D99122_1H7 [MICA/B]_L0, (lv) SEQ ID Nos: 671 and 675 for D99136_2E8 [MICA/B]_H0_D99136_2E8 [MICA/B]_L0, (lvi) SEQ ID Nos: 647 and 651 for D88487_2A8 [MICA/B]_H0_D88487_2A8 [MICA/B]_L0, (lvii) SEQ ID Nos: 679 and 683 for 3F9[MICA/B]_H0_3F9[MICA/B]_L0, (lviii) SEQ ID Nos: 687 and 691 for 6E1[MICA/B]_H0_6E1[MICA/B]_L0, (lix) SEQ ID Nos: 695 and 699 for 7C6[MICA/B]_H0_7C6[MICA/B]_L0, (lx) SEQ ID Nos: 703 and 707 for 13A9 [MICA/B]_H0_13A9 [MICA/B]_L0, and (lxi) SEQ ID Nos: 711 and 715 for 1D5 [MICA/B]_H0_1D5 [MICA/B]_L0, as depicted in FIGS. 21 and 22.

In a further embodiment and in accordance with any of the above, the anti-NKp46 scFv comprises a set of vhCDR1-3 and vlCDR1-3 from a first variable heavy chain VH1 domain and first variable light chain VL1 domain pair selected from the group including: (i) SEQ ID NOs: 720-722 for vhCDR1-3 and SEQ ID NOs: 724-726 for vlCDR1-3 of 2C10A3.372[NKp46]_H1_2C10A3.372[NKp46]_L1, and (ii) SEQ ID NOs: 728-730 for vhCDR1-3 and SEQ ID NOs: 732-734 for vlCDR1-3 of NKp46-A[NKp46]_H_NKp46-A[NKp46]_L, as depicted in FIGS. 23 and 24.

In a further embodiment and in accordance with any of the above, the anti-NKp46 scFv comprises a variable heavy domain and variable light domain pair selected from the group including: (i) SEQ ID Nos: 719 and 723 for 2C10A3.372[NKp46]_H1_2C10A3.372[NKp46]_L1, and (ii) SEQ ID Nos: 727 and 731 for NKp46-A[NKp46]_H_NKp46-A[NKp46]_L, as depicted in FIGS. 23 and 24.

In a further embodiment and in accordance with any of the above, the variable light domain of the anti-NKp46 scFv is covalently attached to the N-terminus of the first Fc domain using a domain linker, or the variable heavy domain of the anti-NKp46 scFv is covalently attached to the N-terminus of the first Fc domain using a domain linker.

In a further embodiment and in accordance with any of the above, the heterodimeric antibody comprises a first amino acid sequence, a second amino acid sequence, and a third amino acid sequence, wherein the first, second, and third amino acid sequences are selected from the group including: (i) the amino acid sequences of SEQ ID NOs: 821-823 of XENP47446, and (ii) the amino acid sequences of SEQ ID NOs: 824-826 of XENP47447, as depicted in FIG. 35.

In a further embodiment and in accordance with any of the above, the scFv linker is a charged scFv linker.

In a further embodiment and in accordance with the above, the scFv linker is a charged scFv linker having the amino acid sequence (GKPGS)₄ (SEQ ID NO: 1).

In a further embodiment and in accordance with any of the above, the first and second Fc domains are each variant Fc domains.

In a further embodiment and in accordance with the above, the first and/or second variant Fc domains comprise one or more FcγRIIIA (CD16a) binding variant substitutions.

In a further embodiment and in accordance with the above, the one or more FcγRIIIA (CD16a) binding variant substitutions are selected from the group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with the above, the first and second variant Fc domains comprise a set of FcγRIIIA (CD16a) binding variant substitutions selected from the group including: (i) S239D/I332E: S239D/I332E, (ii) S239D: S239D, (iii) I332E: I332E, (iv) WT: S239D/I332E, (v) WT: S239D, (vi) WT: 1332E, (vii) S239D/I332E: WT, (viii) S239D: WT, (ix) 1332E: WT, (x) S239D/I332E: S239D, (xi) S239D/I332E: 1332E, (xii) S239D: S239D/I332E, (xiii) 1332E: S239D/I332E, (xiv) S239D: 1332E, and (xv) 1332E: S239D, wherein numbering is according to EU numbering.

In a further embodiment and in accordance with any of the above, the first and/or second variant Fc domains comprise the FcγRIIIA (CD16a) binding variant substitutions of S239D/I332E, wherein numbering is according to EU numbering.

In a further embodiment and in accordance with any of the above, the first and second variant Fc domains comprise a set of heterodimerization variants selected from the group including those depicted in FIGS. 4A-4F, wherein numbering is according to EU numbering.

In a further embodiment and in accordance with the above, the set of heterodimerization variants is selected from the group including: (i) S364K/E357Q: L368D/K370S, (ii) S364K: L368D/K370S, (iii) S364K: L368E/K370S, (iv) D401K: T411E/K360E/Q362E, and (v) T366W: T366S/L368A/Y407V, wherein numbering is according to EU numbering.

In a further embodiment and in accordance with any of the above, the first and second variant Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant Fc domain comprises one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first monomer comprises amino acid variants S364K/E357Q/E233P/L234V/L235A/G236del/S267K, wherein the second monomer comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second monomers each further comprise amino acid variants M428L/N434S, M428L/434A, or M252Y/S254T/T256E, wherein numbering is according to EU numbering.

In another aspect, a nucleic acid composition comprising nucleic acids encoding the first and second monomers and the light chain of the antibody (as described above) is provided.

In another aspect, an expression vector comprising the nucleic acids (as described above) is provided.

In another aspect, a host cell transformed with the expression vector (as described above) is provided.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, the present disclosure provides a heterodimeric antibody, comprising: (a) a first monomer comprising, from N-terminus to C-terminus, a VH1-CH1-hinge-CH2-CH3-domain linker-scFv, wherein VH1 is a first variable heavy domain, scFv is an anti-MICA/B scFv, and CH2-CH3 is a first Fc domain; (b) a second monomer comprising, from N-terminus to C-terminus, a VH2-CH1-hinge-CH2-CH3, wherein CH2-CH3 is a second Fc domain; and (c) a light chain comprising, from N-terminus to C-terminus, a VL1-CL, wherein VL1 is a first variable light domain and CL is a constant light domain, wherein: (i) the VH1 and the VL1 form a first NKp46 antigen binding domain, (ii) the VH2 domain and the VL1 form a second NKp46 antigen binding domain, and (iii) the anti-NKp46 scFv comprises a VH3 domain, a scFv linker, and a VL2 domain.

In a further embodiment and in accordance with the above, the first and/or second NKp46 antigen binding domains comprise a set of vhCDR1-3 and vlCDR1-3 from a variable heavy domain and variable light domain pair selected from the group including: (i) SEQ ID NOs: 720-722 for vhCDR1-3 and SEQ ID NOs: 724-726 for vlCDR1-3 of 2C10A3.372[NKp46]_H1_2C10A3.372[NKp46]_L1, and (ii) SEQ ID NOs: 728-730 for vhCDR1-3 and SEQ ID NOs: 732-734 for vlCDR1-3 of NKp46-A[NKp46]_H_NKp46-A[NKp46]_L, as depicted in FIGS. 23 and 24.

In a further embodiment and in accordance with any of the above, the first and/or second NKp46 antigen binding domains comprise a variable heavy domain and variable light domain pair selected from the group including: (i) SEQ ID Nos: 719 and 723 for 2C10A3.372[NKp46]_H1_2C10A3.372[NKp46]_L1, and (ii) SEQ ID Nos: 727 and 731 for NKp46-A[NKp46]_H_NKp46-A[NKp46]_L, as depicted in FIGS. 23 and 24.

In a further embodiment and in accordance with any of the above, the anti-MICA/B scFv comprises a set of vhCDR1-3 and vlCDR1-3 from a first variable heavy chain VH1 domain and first variable light chain VL1 domain pair selected from the group including: (i) SEQ ID NOs: 240-242 for vhCDR1-3 and SEQ ID NOs: 244-246 for vlCDR1-3 of D94837_1E1 11 [MICA/B]_H0_D94837_E11_1 [MICA/B]_L0, (ii) SEQ ID NOs: 248-250 for vhCDR1-3 and SEQ ID NOs: 252-254 for v1CDR1-3 of D94837_1E11_1 [MICA/B]_H1_D94837_1E11_1 [MICA/B]_L1, (iii) SEQ ID NOs: 256-258 for vhCDR1-3 and SEQ ID NOs: 260-262 for v1CDR1-3 of D94837_E11_1 [MICA/B]_H1_D94837_1E11_1 [MICA/B]_L2, (iv) SEQ ID NOs: 264-266 for vhCDR1-3 and SEQ ID NOs: 268-270 for v1CDR1-3 of D94837_1E11_1 [MICA/B]_H2_D94837_1E11_1 [MICA/B]_L1, (v) SEQ ID NOs: 272-274 for vhCDR1-3 and SEQ ID NOs: 276-278 for v1CDR1-3 of D94837_1E11_1 [MICA/B]_H2_D94837_1E11_1 [MICA/B]_L2, (vi) SEQ ID NOs: 280-282 for vhCDR1-3 and SEQ ID NOs: 284-286 for v1CDR1-3 of 2E5 [MICA/B]_H0_2E5 [MICA/B]_L0, (vii) SEQ ID NOs: 288-290 for vhCDR1-3 and SEQ ID NOs: 292-294 for v1CDR1-3 of 2E5 [MICA/B]_H1_2E5 [MICA/B]_L1, (viii) SEQ ID NOs: 296-298 for vhCDR1-3 and SEQ ID NOs: 300-302 for v1CDR1-3 of 2E5 [MICA/B]_H1_2E5 [MICA/B]_L2, (ix) SEQ ID NOs: 304-306 for vhCDR1-3 and SEQ ID NOs: 308-310 for v1CDR1-3 of 2E5 [MICA/B]_H2_2E5 [MICA/B]_L1, (x) SEQ ID NOs: 312-314 for vhCDR1-3 and SEQ ID NOs: 316-318 for v1CDR1-3 of 2E5 [MICA/B]_H2_2E5 [MICA/B]_L2, (xi) SEQ ID NOs: 320-322 for vhCDR1-3 and SEQ ID NOs: 324-326 for v1CDR1-3 of D94852_2E12 [MICA/B]_H0_D94852_2E12 [MICA/B]_L0, (xii) SEQ ID NOs: 328-330 for vhCDR1-3 and SEQ ID NOs: 332-334 for v1CDR1-3 of D94852_2E12 [MICA/B]_H1_D94852_2E12 [MICA/B]_L1, (xiii) SEQ ID NOs: 336-338 for vhCDR1-3 and SEQ ID NOs: 340-342 for v1CDR1-3 of D94852_2E12 [MICA/B]_H1_D94852_2E12 [MICA/B]_L2, (xiv) SEQ ID NOs: 344-346 for vhCDR1-3 and SEQ ID NOs: 348-350 for v1CDR1-3 of D94852_2E12 [MICA/B]_H2_D94852_2E12 [MICA/B]_L1, (xv) SEQ ID NOs: 352-354 for vhCDR1-3 and SEQ ID NOs: 356-358 for v1CDR1-3 of D94852_2E12 [MICA/B]_H2_D94852_2E12 [MICA/B]_L2, (xvi) SEQ ID NOs: 360-362 for vhCDR1-3 and SEQ ID NOs: 364-366 for v1CDR1-3 of D99136_2F7 [MICA/B]_H0_D99136_2F7 [MICA/B]_L0, (xvii) SEQ ID NOs: 368-370 for vhCDR1-3 and SEQ ID NOs: 372-374 for v1CDR1-3 of D99136_2F7 [MICA/B]_H1_D99136_2F7 [MICA/B]_L1, (xviii) SEQ ID NOs: 376-378 for vhCDR1-3 and SEQ ID NOs: 380-382 for v1CDR1-3 of D99136_2F7 [MICA/B]_H1_D99136_2F7 [MICA/B]_L2, (xix) SEQ ID NOs: 384-386 for vhCDR1-3 and SEQ ID NOs: 388-390 for v1CDR1-3 of D99136_2F7 [MICA/B]_H2_D99136_2F7 [MICA/B]_L1, (xx) SEQ ID NOs: 392-394 for vhCDR1-3 and SEQ ID NOs: 396-398 for v1CDR1-3 of D99136_2F7 [MICA/B]_H2_D99136_2F7 [MICA/B]_L2, (xxi) SEQ ID NOs: 400-402 for vhCDR1-3 and SEQ ID NOs: 404-406 for v1CDR1-3 of D103388_1C7 [MICA/B]_H0_D103388_1C7 [MICA/B]_L0, (xxii) SEQ ID NOs: 408-410 for vhCDR1-3 and SEQ ID NOs: 412-414 for v1CDR1-3 of D103388_1C7 [MICA/B]_H1_D103388_1C7 [MICA/B]_L1, (xxiii) SEQ ID NOs: 416-418 for vhCDR1-3 and SEQ ID NOs: 420-422 for v1CDR1-3 of D103388_1C7 [MICA/B]_H1_D103388_1C7 [MICA/B]_L2, (xxiv) SEQ ID NOs: 424-426 for vhCDR1-3 and SEQ ID NOs: 428-430 for v1CDR1-3 of D103388_1C7 [MICA/B]_H2_D103388_1C7 [MICA/B]_L1, (xxv) SEQ ID NOs: 432-434 for vhCDR1-3 and SEQ ID NOs: 436-438 for v1CDR1-3 of D103388_1C7 [MICA/B]_H2_D103388_1C7 [MICA/B]_L2, (xxvi) SEQ ID NOs: 440-442 for vhCDR1-3 and SEQ ID NOs: 444-446 for v1CDR1-3 of D103388_1D7 [MICA/B]_H0_D103388_1D7 [MICA/B]_L0, (xxvii) SEQ ID NOs: 448-450 for vhCDR1-3 and SEQ ID NOs: 452-454 for v1CDR1-3 of D103388_1D7 [MICA/B]_H1_D103388_1D7 [MICA/B]_L1, (xxviii) SEQ ID NOs: 456-458 for vhCDR1-3 and SEQ ID NOs: 460-462 for v1CDR1-3 of D103388_1D7 [MICA/B]_H1_D103388_1D7 [MICA/B]_L2, (xxix) SEQ ID NOs: 464-466 for vhCDR1-3 and SEQ ID NOs: 468-470 for v1CDR1-3 of D103388_1D7 [MICA/B]_H2_D103388_1D7 [MICA/B]_L1, (xxx) SEQ ID NOs: 472-474 for vhCDR1-3 and SEQ ID NOs: 476-478 for v1CDR1-3 of D103388_1D7 [MICA/B]_H2_D103388_1D7 [MICA/B]_L2, (xxxi) SEQ ID NOs: 480-482 for vhCDR1-3 and SEQ ID NOs: 484-486 for v1CDR1-3 of D105317_1A2 [MICA/B]_H0_D105317_1A2 [MICA/B]_L0, (xxxii) SEQ ID NOs: 488-490 for vhCDR1-3 and SEQ ID NOs: 492-494 for v1CDR1-3 of D105317_1A2 [MICA/B]_H1_D105317_1A2 [MICA/B]_L1, (xxxiii) SEQ ID NOs: 496-498 for vhCDR1-3 and SEQ ID NOs: 500-502 for v1CDR1-3 of D105317_1A2 [MICA/B]_H1_D105317_1A2 [MICA/B]_L2, (xxxiv) SEQ ID NOs: 504-506 for vhCDR1-3 and SEQ ID NOs: 508-510 for vlCDR1-3 of D105317_1A2 [MICA/B]_H2_D105317_1A2 [MICA/B]_L1, (xxxv) SEQ ID NOs: 512-514 for vhCDR1-3 and SEQ ID NOs: 516-518 for v1CDR1-3 of D105317_1A2 [MICA/B]_H2_D105317_1A2 [MICA/B]_L2, (xxxvi) SEQ ID NOs: 520-522 for vhCDR1-3 and SEQ ID NOs: 524-526 for v1CDR1-3 of D99136_2C11 [MICA/B]_H0_D99136_2C11 [MICA/B]_L0, (xxxvii) SEQ ID NOs: 528-530 for vhCDR1-3 and SEQ ID NOs: 532-534 for vlCDR1-3 of D99136_2C11 [MICA/B]_H1_D99136_2C11 [MICA/B]_L1, (xxxviii) SEQ ID NOs: 536-538 for vhCDR1-3 and SEQ ID NOs: 540-542 for v1CDR1-3 of D99136_2C11 [MICA/B]_H1_D99136_2C11 [MICA/B]_L2, (xxxix) SEQ ID NOs: 544-546 for vhCDR1-3 and SEQ ID NOs: 548-550 for v1CDR1-3 of D99136_2C11 [MICA/B]_H2_D99136_2C11 [MICA/B]_L1, (xl) SEQ ID NOs: 552-554 for vhCDR1-3 and SEQ ID NOs: 556-558 for vlCDR1-3 of D99136_2C11 [MICA/B]_H2_D99136_2C11 [MICA/B]_L2, (xli) SEQ ID NOs: 560-562 for vhCDR1-3 and SEQ ID NOs: 564-566 for v1CDR1-3 of D103388_1B11-2 [MICA/B]_H0_D103388_1B11-2 [MICA/B]_L0, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (lvii) SEQ ID NOs: 680-682 for vhCDR1-3 and SEQ ID NOs: 684-686 for vlCDR1-3 of 3F9[MICA/B]_H0_3F9[MICA/B]_L0, (lviii) SEQ ID NOs: 688-690 for vhCDR1-3 and SEQ ID NOs: 692-694 for vlCDR1-3 of 6E1[MICA/B]_H0_6E1[MICA/B]_L0, (lix) SEQ ID NOs: 696-698 for vhCDR1-3 and SEQ ID NOs: 700-702 for vlCDR1-3 of 7C6[MICA/B]_H0_7C6[MICA/B]_L0, (lx) SEQ ID NOs: 704-706 for vhCDR1-3 and SEQ ID NOs: 708-710 for vlCDR1-3 of 13A9 [MICA/B]_H0_13A9 [MICA/B]_L0, and (lxi) SEQ ID NOs: 712-714 for vhCDR1-3 and SEQ ID NOs: 716-718 for vlCDR1-3 of 1D5 [MICA/B]_H0_1D5 [MICA/B]_L0, as depicted in FIGS. 21 and 22.

In a further embodiment and in accordance with any of the above, the anti-MICA/B scFv comprises a variable heavy domain and variable light domain pair selected from the group including: (i) SEQ ID Nos: 239 and 243 for D94837_E11_1 [MICA/B]_H0_D94837_1E11_1 [MICA/B]_L0, (ii) SEQ ID Nos: 247 and 251 for D94837_1E11_1 [MICA/B]_H1_D94837_1E11_1 [MICA/B]_L1, (iii) SEQ ID Nos: 255 and 259 for D94837_1E1_1 [MICA/B]_H1_D94837_E11_1 [MICA/B]_L2, (iv) SEQ ID Nos: 263 and 267 for D94837_1E11_1 [MICA/B]_H2_D94837_1E11_1 [MICA/B]_L1, (v) SEQ ID Nos: 271 and 275 for D94837_E11_1 [MICA/B]_H2_D94837_E11_1 [MICA/B]_L2, (vi) SEQ ID Nos: 279 and 283 for 2E5 [MICA/B]_H0_2E5 [MICA/B]_L0, (vii) SEQ ID Nos: 287 and 291 for 2E5 [MICA/B]_H1_2E5 [MICA/B]_L1, (viii) SEQ ID Nos: 295 and 299 for 2E5 [MICA/B]_H1_2E5 [MICA/B]_L2, (ix) SEQ ID Nos: 303 and 307 for 2E5 [MICA/B]_H2_2E5 [MICA/B]_L1, (x) SEQ ID Nos: 311 and 315 for 2E5 [MICA/B]_H2_2E5 [MICA/B]_L2, (xi) SEQ ID Nos: 319 and 323 for D94852_2E12 [MICA/B]_H0_D94852_2E12 [MICA/B]_L0, (xii) SEQ ID Nos: 327 and 331 for D94852_2E12 [MICA/B]_H1_D94852_2E12 [MICA/B]_L1, (xiii) SEQ ID Nos: 335 and 339 for D94852_2E12 [MICA/B]_H1_D94852_2E12 [MICA/B]_L2, (xiv) SEQ ID Nos: 343 and 347 for D94852_2E12 [MICA/B]_H2_D94852_2E12 [MICA/B]_L1, (xv) SEQ ID Nos: 351 and 355 for D94852_2E12 [MICA/B]_H2_D94852_2E12 [MICA/B]_L2, (xvi) SEQ ID Nos: 359 and 363 for D99136_2F7 [MICA/B]_H0_D99136_2F7 [MICA/B]_L0, (xvii) SEQ ID Nos: 367 and 371 for D99136_2F7 [MICA/B]_H1_D99136_2F7 [MICA/B]_L1, (xviii) SEQ ID Nos: 375 and 379 for D99136_2F7 [MICA/B]_H1_D99136_2F7 [MICA/B]_L2, (xix) SEQ ID Nos: 383 and 387 for D99136_2F7 [MICA/B]_H2_D99136_2F7 [MICA/B]_L1, (xx) SEQ ID Nos: 391 and 395 for D99136_2F7 [MICA/B]_H2_D99136_2F7 [MICA/B]_L2, (xxi) SEQ ID Nos: 399 and 403 for D103388_1C7 [MICA/B]_H0_D103388_1C7 [MICA/B]_L0, (xxii) SEQ ID Nos: 407 and 411 for D103388_1C7 [MICA/B]_H1_D103388_1C7 [MICA/B]_L1, (xxiii) SEQ ID Nos: 415 and 419 for D103388_1C7 [MICA/B]_H1_D103388_1C7 [MICA/B]_L2, (xxiv) SEQ ID Nos: 423 and 427 for D103388_1C7 [MICA/B]_H2_D103388_1C7 [MICA/B]_L1, (xxv) SEQ ID Nos: 431 and 435 for D103388_1C7 [MICA/B]_H2_D103388_1C7 [MICA/B]_L2, (xxvi) SEQ ID Nos: 439 and 443 for D103388_1D7 [MICA/B]_H0_D103388_1D7 [MICA/B]_L0, (xxvii) SEQ ID Nos: 447 and 451 for D103388_1D7 [MICA/B]_H1_D103388_1D7 [MICA/B]_L1, (xxviii) SEQ ID Nos: 455 and 459 for D103388_1D7 [MICA/B]_H1_D103388_1D7 [MICA/B]_L2, (xxix) SEQ ID Nos: 463 and 467 for D103388_1D7 [MICA/B]_H2_D103388_1D7 [MICA/B]_L1, (xxx) SEQ ID Nos: 471 and 475 for D103388_1D7 [MICA/B]_H2_D103388_1D7 [MICA/B]_L2, (xxxi) SEQ ID Nos: 479 and 483 for D105317_1A2 [MICA/B]_H0_D105317_1A2 [MICA/B]_L0, (xxxii) SEQ ID Nos: 487 and 491 for D105317_1A2 [MICA/B]_H1_D105317_1A2 [MICA/B]_L1, (xxxiii) SEQ ID Nos: 495 and 499 for D105317_1A2 [MICA/B]_H1_D105317_1A2 [MICA/B]_L2, (xxxiv) SEQ ID Nos: 503 and 507 for D105317_1A2 [MICA/B]_H2_D105317_1A2 [MICA/B]_L1, (xxxv) SEQ ID Nos: 511 and 515 for D105317_1A2 [MICA/B]_H2_D105317_1A2 [MICA/B]_L2, (xxxvi) SEQ ID Nos: 519 and 523 for D99136_2C11 [MICA/B]_H0_D99136_2C11 [MICA/B]_L0, (xxxvii) SEQ ID Nos: 527 and 531 for D99136_2C11 [MICA/B]_H1_D99136_2C11 [MICA/B]_L1, (xxxviii) SEQ ID Nos: 535 and 539 for D99136_2C11 [MICA/B]_H1_D99136_2C11 [MICA/B]_L2, (xxxix) SEQ ID Nos: 543 and 547 for D99136_2C11 [MICA/B]_H2_D99136_2C11 [MICA/B]_L1, (xl) SEQ ID Nos: 551 and 555 for D99136_2C11 [MICA/B]_H2_D99136_2C11 [MICA/B]_L2, (xli) SEQ ID Nos: 559 and 563 for D103388_1B11-2 [MICA/B]_H0_D103388_1B11-2 [MICA/B]_L0, (xlii) SEQ ID Nos: 567 and 571 for D105317_1B6 [MICA/B]_H0_D105317_1B6 [MICA/B]_L0, (xliii) SEQ ID Nos: 575 and 579 for D105317_1C8 [MICA/B]_H0_D105317_1C8 [MICA/B]_L0, (xliv) SEQ ID Nos: 583 and 587 for D105317_1F6 [MICA/B]_H0_D105317_1F6 [MICA/B]_L0, (xlv) SEQ ID Nos: 591 and 595 for D105317_1F7 [MICA/B]_H0_D105317_1F7 [MICA/B]_L0, (xlvi) SEQ ID Nos: 599 and 603 for D94837_1D3 [MICA/B]_H0_D94837_1D3 [MICA/B]_L0, (xlvii) SEQ ID Nos: 607 and 611 for D94837_1D8 [MICA/B]_H0_D94837_1D8 [MICA/B]_L0, (xlviii) SEQ ID Nos: 615 and 619 for D94837_1D9 [MICA/B]_H0_D94837_1D9 [MICA/B]_L0, (xlix) SEQ ID Nos: 623 and 627 for D94837_1E1 [MICA/B]_H0_D94837_1E1 [MICA/B]_L0, (1) SEQ ID Nos: 631 and 635 for D94852_2D4 [MICA/B]_H0_D94852_2D4 [MICA/B]_L0, (li) SEQ ID Nos: 639 and 643 for D94852_2G8 [MICA/B]_H0_D94852_2G8 [MICA/B]_L0, (lii) SEQ ID Nos: 647 and 651 for D88487_2A8 [MICA/B]_H0_D88487_2A8 [MICA/B]_L0, (liii) SEQ ID Nos: 655 and 659 for D94837_3B12 [MICA/B]_H0_D94837_3B12 [MICA/B]_L0, (liv) SEQ ID Nos: 663 and 667 for D99122_1H7 [MICA/B]_H0_D99122_1H7 [MICA/B]_L0, (lv) SEQ ID Nos: 671 and 675 for D99136_2E8 [MICA/B]_H0_D99136_2E8 [MICA/B]_L0, (lvi) SEQ ID Nos: 647 and 651 for D88487_2A8 [MICA/B]_H0_D88487_2A8 [MICA/B]_L0, (lvii) SEQ ID Nos: 679 and 683 for 3F9[MICA/B]_H0_3F9[MICA/B]_L0, (lviii) SEQ ID Nos: 687 and 691 for 6E1[MICA/B]_H0_6E1[MICA/B]_L0, (lix) SEQ ID Nos: 695 and 699 for 7C6[MICA/B]_H0_7C6[MICA/B]_L0, (lx) SEQ ID Nos: 703 and 707 for 13A9 [MICA/B]_H0_13A9 [MICA/B]_L0, and (lxi) SEQ ID Nos: 711 and 715 for 1D5 [MICA/B]_H0_1D5 [MICA/B]_L0, as depicted in FIGS. 21 and 22.

In a further embodiment and in accordance with any of the above, the variable light domain of the anti-MICA/B scFv is covalently attached to the N-terminus of the first Fc domain using a domain linker, or the variable heavy domain of the anti-MICA/B scFv is covalently attached to the N-terminus of the first Fc domain using a domain linker.

In a further embodiment and in accordance with any of the above, the scFv linker is a charged scFv linker.

In a further embodiment and in accordance with the above, the scFv linker is a charged scFv linker having the amino acid sequence (GKPGS)₄ (SEQ ID NO: 1).

In a further embodiment and in accordance with any of the above, the first and second Fc domains are each variant Fc domains.

In a further embodiment and in accordance with the above, the first and/or second variant Fc domains comprise one or more FcγRIIIA (CD16a) binding variant substitutions.

In a further embodiment and in accordance with the above, the one or more FcγRIIIA (CD16a) binding variant substitutions are selected from the group including: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

In a further embodiment and in accordance with the above, the first and second variant Fc domains comprise a set of FcγRIIIA (CD16a) binding variant substitutions selected from the group including: (i) S239D/I332E: S239D/I332E, (ii) S239D: S239D, (iii) 1332E: 1332E, (iv) WT: S239D/I332E, (v) WT: S239D, (vi) WT: 1332E, (vii) S239D/I332E: WT, (viii) S239D: WT, (ix) 1332E: WT, (x) S239D/I332E: S239D, (xi) S239D/I332E: 1332E, (xii) S239D: S239D/I332E, (xiii) 1332E: S239D/I332E, (xiv) S239D: 1332E, and (xv) 1332E: S239D, wherein numbering is according to EU numbering.

In a further embodiment and in accordance with any of the above, the first and/or second variant Fc domains comprise the FcγRIIIA (CD16a) binding variant substitutions of S239D/I332E, wherein numbering is according to EU numbering.

In a further embodiment and in accordance with any of the above, the first and second variant Fc domains comprise a set of heterodimerization variants selected from the group including those depicted in FIGS. 4A-4F, wherein numbering is according to EU numbering.

In a further embodiment and in accordance with the above, the set of heterodimerization variants is selected from the group including: (i) S364K/E357Q: L368D/K370S, (ii) S364K: L368D/K370S, (iii) S364K: L368E/K370S, (iv) D401K: T411E/K360E/Q362E, and (v) T366W: T366S/L368A/Y407V, wherein numbering is according to EU numbering.

In a further embodiment and in accordance with any of the above, the first and second variant Fc domains further comprise one or more ablation variants.

In a further embodiment and in accordance with the above, the one or more ablation variants are E233P/L234V/L235A/G236del/S267K, wherein numbering is according to EU numbering.

In a further embodiment and in accordance with any of the above, one of the first or second variant Fc domain comprises one or more pI variants.

In a further embodiment and in accordance with the above, the one or more pI variants are N208D/Q295E/N384D/Q418E/N421D, wherein numbering is according to EU numbering.

In a further embodiment and in accordance with any of the above, the first monomer comprises amino acid variants S364K/E357Q/E233P/L234V/L235A/G236del/S267K, wherein the second monomer comprises amino acid variants L368D/K370S/N208D/Q295E/N384D/Q418E/N421D/E233P/L234V/L235A/G236del/S267K, and wherein numbering is according to EU numbering.

In a further embodiment and in accordance with any of the above, the first and second monomers each further comprise amino acid variants M428L/N434S, M428L/434A, or M252Y/S254T/T256E, wherein numbering is according to EU numbering.

In another aspect, a nucleic acid composition comprising nucleic acids encoding the first and second monomers and the light chain of the antibody (as described above) is provided.

In another aspect, an expression vector comprising the nucleic acids (as described above) is provided.

In another aspect, a host cell transformed with the expression vector (as described above) is provided.

In another aspect, a method of making a heterodimeric antibody is provided, the method comprising: (a) culturing the host cell (as described above) under conditions wherein the heterodimeric antibody is expressed, and (b) recovering the heterodimeric antibody.

In another aspect, the present disclosure provides a method of treating cancer, comprising: administering to a human subject an antibody comprising: (i) a MICA/B antigen binding domain (as described above), (ii) a NKp46 antigen binding domain (as described above), or (iii) a heterodimeric antibody (as described above) to the human subject.

In a further embodiment and in accordance with the above, the method can further comprise administering an IL-15-Fc fusion protein to the human subject.

In a further embodiment and in accordance with the above, the IL-15 Fc fusion protein comprises the amino acid sequences of SEQ ID NOs: 833-834 of XENP24045.

In another aspect, the present disclosure provides a method of blocking MICA/B shedding by NK cells in a human subject, the method comprising: administering to the human subject a therapeutically effective amount of an antibody comprising: (i) a MICA/B antigen binding domain (as described above), or (ii) a heterodimeric antibody (as described above) to the human subject.

In a further embodiment and in accordance with the above, CD16 and/or NKG2D is activated on the NK cells.

In a further embodiment and in accordance with any of the above, the method further comprises administering an IL-15-Fc fusion protein to the human subject.

In a further embodiment and in accordance with the above, the IL-15 Fc fusion protein comprises the amino acid sequences of SEQ ID NOs: 833-834 of XENP24045.

In another aspect, the present disclosure provides a heterodimeric antibody comprising: (a) a first monomer; (b) a second monomer; and (c) a third monomer, wherein the first monomer, the second monomer, and the third monomer comprise: (i) the amino acid sequences of SEQ ID NOs: 859-861 of XENP44543, respectively, (ii) the amino acid sequences of SEQ ID NOs: 862-864 of XENP45903, respectively, (iii) the amino acid sequences of SEQ ID NOs: 865-867 of XENP45902, respectively, (iv) the amino acid sequences of SEQ ID NOs: 868-870 of XENP44549, respectively, (v) the amino acid sequences of SEQ ID NOs: 871-873 of XENP44551, respectively, (vi) the amino acid sequences of SEQ ID NOs: 874-876 of XENP44553, respectively, or (vii) the amino acid sequences of SEQ ID NOs: 877-879 of XENP44552, respectively, as depicted in FIG. 71.

In another aspect, the present disclosure provides a nucleic acid composition comprising: (a) a first nucleic acid encoding the first monomer of a heterodimeric antibody (as described above), (b) a second nucleic acid encoding the second monomer of a heterodimeric antibody (as described above), and (c) a third nucleic acid encoding the third monomer of a heterodimeric antibody (as described above).

In another aspect, the present disclosure provides an expression vector composition comprising: (a) a first expression vector comprising the first nucleic acid encoding the first monomer of a heterodimeric antibody (as described above), (b) a second expression vector comprising the second nucleic acid encoding the second monomer of a heterodimeric antibody (as described above), and (c) a third expression vector comprising the third nucleic acid encoding the third monomer of a heterodimeric antibody (as described above).

In another aspect, the present disclosure provides a host cell comprising an expression vector composition (as described above).

In another aspect, the present disclosure provides a method of making a heterodimeric antibody, comprising: (a) culturing a host cell (as described above) under conditions wherein the heterodimeric antibody is expressed; and (b) recovering the heterodimeric antibody.

In another aspect, the present disclosure provides a method of treating cancer, comprising: administering to a human subject an antibody comprising a heterodimeric antibody (as described above).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “FIG.,” “FIG.,” “Figure,” “Figures,” “FIGS.,” and “FIGS.” herein) of which.

FIG. 1 depicts the sequences for human and cynomolgus MICA. Such MICA are useful for the development of cross-reactive MICA antigen binding domains for ease of clinical development. The following alleles of MICA are known: MICA*001, MICA*002, MICA*004, MICA*005, MICA*006, MICA*007, MICA*008, MICA*009, MICA*010, MICA*011, MICA*012, MICA*013, MICA*014, MICA*015, MICA*016, MICA*017, MICA*018, MICA*019, MICA*020, MICA*022, MICA*023, MICA*024, MICA*025, MICA*026, MICA*027, MICA*028, MICA*029, MICA*030, MICA*031, MICA*032, MICA*033, MICA*034, MICA*035, MICA*036, MICA*037, MICA*038, MICA*039, MICA*040, MICA*041, MICA*042, MICA*043, MICA*044, MICA*045, MICA*046, MICA*047, MICA*048, MICA*049, MICA*050, MICA*051, MICA*052, MICA*053, MICA*054, MICA*055 and MICA*056.

FIG. 2 depicts the sequences for human and cynomolgus MICB. Such MICB are useful for the development of cross-reactive MICA antigen binding domains for ease of clinical development. The following alleles of MICB are known: MICB*001, MICB*002, MICB*003, MICB*004, MICB*005, MICB*006, MICB*007, MICB*008, MICB*009N, MICB*010, MICB*011, MICB*012, MICB*013, MICB*014, MICB*015, MICB*016, MICB*018, MICB*019, MICB*020, MICB*021N and MICB*022. MICB*009N and MICB*021N are null alleles which are not expressed. The three most common MICB alleles in the human population could be MICB*005, MICB*004, and MICB*002.

FIG. 3 depicts the sequences for human, mouse, and cynomolgus NKp46. Such NKp46 are useful for the development of cross-reactive NKp46 antigen binding domains for ease of clinical development.

FIG. 4 (FIGS. 4A-4F) depicts useful pairs of heterodimerization variant sets (including skew and pI variants). In FIG. 4F, there are variants for which there are no corresponding “monomer 2” variants. Such variants are pI variants that can be used alone on either monomer of a NKp46×MICA/B bsAb, or included, for example, on the non-scFv side of a format that utilizes an scFv as a component and an appropriate charged scFv linker can be used on the second monomer that utilizes an scFv as a binding domain. Suitable charged linkers are shown in FIG. 8.

FIG. 5 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein.

FIG. 6 depicts useful ablation variants that ablate FcγR binding (also referred to as “knockouts” or “KO” variants). In some embodiments, such ablation variants are included in the Fc domain of both monomers of the subject antibody described herein. In other embodiments, the ablation variants are only included on only one variant Fc domain.

FIG. 7 depicts useful variants that enhance FcγR binding (also referred to as ADCC-enhanced variants). In some embodiments, such ADCC-enhanced variants are included in the Fc domain of both monomers of the subject antibody described herein. In other embodiments, the variants are only included on only one variant Fc domain.

FIG. 8 depicts a number of charged scFv linkers that find use in increasing or decreasing the pI of the subject heterodimeric NKp46×MICA/B bsAbs that utilize one or more scFv as a component, as described herein. The (+H) positive linker finds particular use herein. A single prior art scFv linker with a single charge is referenced as “Whitlow”, from Whitlow et al., Protein Engineering 6(8):989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc and 2+1 Fab₂-scFv-Fc formats).

FIG. 9 depicts a number of exemplary domain linkers. In some embodiments, these linkers find use linking a single-chain Fv to an Fc chain. In some embodiments, these linkers may be combined in any orientation. For example, a GGGGS linker (SEQ ID NO: 2) may be combined with a “lower half hinge” linker at the N-terminus or at the C-terminus.

FIG. 10 shows a particularly useful embodiment of the heterodimeric Fc domains (i.e. CH2-CH3 in this embodiment) of the NKp46×MICA/B bsAbs of the invention.

FIG. 11 depicts various heterodimeric skewing variant amino acid substitutions that can be used with the heterodimeric antibodies described herein.

FIG. 12 (FIGS. 12A-12E) shows the sequences of heterodimeric NKp46×MICA/B bsAb backbones with ablated effector function. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants on both chains. Heterodimeric Fc backbone 13 is based on human IgG1 (356E/358M allotype), and includes the T366W skew variant on a first heterodimeric Fc chain, the T366S/L368A/Y407V skew variants and H435R/Y436F purification variants on a second heterodimeric Fc chain, and the L234A/L235A/D265S ablation variants on both chains. Heterodimeric Fc backbone 14 is based on human IgG1 (356E/358M allotype), and includes the T366W skew variant on a first heterodimeric Fc chain, the T366S/L368A/Y407V skew variants and H435R/Y436F purification variants on a second heterodimeric Fc chain, and the L234A/L235A/D265S ablation variants and M252Y/S254T/T256E half-life extension variants on both chains. Heterodimeric Fc backbone 15 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 16 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 17 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIG. 13 (FIGS. 13A-13C) shows the sequences of several useful heterodimeric NKp46×MICA/B bsAb backbones based on human IgG1 and having WT effector function. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and N297S variant that removes glycosylation on both chains. Heterodimeric Fc backbone 8 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and M428L/N434S Xtend variants on both chains. Heterodimeric Fc backbone 9 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 10 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and M428L/N434A Xtend variants on both chains. Heterodimeric Fc backbone 11 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

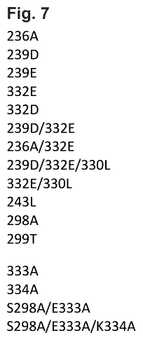

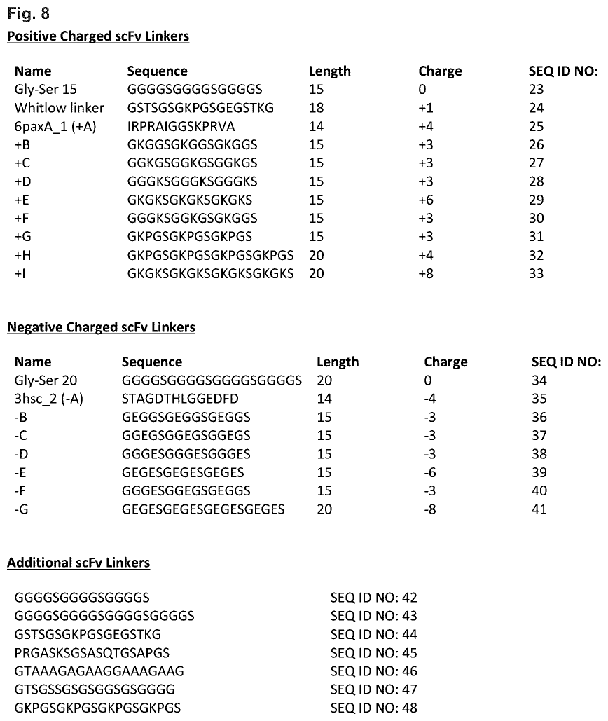

FIG. 14 (FIGS. 14A-14C) shows the sequences of several useful heterodimeric NKp46×MICA/B bsAb backbones based on human IgG1 and having enhanced ADCC function. The sequences here are based on heterodimeric Fc backbone 1 in FIG. 13, although the ADCC variants in FIG. 7 may also be included in any of the other heterodimeric Fc backbones in FIG. 13. ADCC-enhanced Heterodimeric Backbone 1 includes S239D/I332E on both the first and the second heterodimeric Fc chain. ADCC-enhanced Heterodimeric Backbone 2 includes S239D on both the first and the second heterodimeric Fc chain. ADCC-enhanced Heterodimeric Backbone 3 includes 1332E on both the first and the second heterodimeric Fc chain. ADCC-enhanced Heterodimeric Backbone 4 includes S239D/I332E on the second heterodimeric Fc chain. ADCC-enhanced Heterodimeric Backbone 5 includes S239D on the second heterodimeric Fc chain. ADCC-enhanced Heterodimeric Backbone 6 includes 1332E on the second heterodimeric Fc chain. ADCC-enhanced Heterodimeric Backbone 7 includes S239D/I332E on the first heterodimeric Fc chain. ADCC-enhanced Heterodimeric Backbone 8 includes S239D on the first heterodimeric Fc chain. ADCC-enhanced Heterodimeric Backbone 9 includes 1332E on the first heterodimeric Fc chain. ADCC-enhanced Heterodimeric Backbone 10 includes S239D/I332E on the first heterodimeric Fc chain and S239D on the second heterodimeric Fc chain. ADCC-enhanced Heterodimeric Backbone 11 includes S239D/I332E on the first heterodimeric Fc chain and 1332E on the second heterodimeric Fc chain. ADCC-enhanced Heterodimeric Backbone 12 includes S239D on the first heterodimeric Fc chain and S239D/I332E on the second heterodimeric Fc chain. ADCC-enhanced Heterodimeric Backbone 13 includes 1332E on the first heterodimeric Fc chain and S239D/I332E on the second heterodimeric Fc chain. ADCC-enhanced Heterodimeric Backbone 14 includes S239D on the first heterodimeric Fc chain and 1332E on the second heterodimeric Fc chain. ADCC-enhanced Heterodimeric Backbone 15 includes 1332E on the first heterodimeric Fc chain and S239D on the second heterodimeric Fc chain.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1. That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIG. 15 (FIGS. 15A-15C) shows the sequences of several useful heterodimeric NKp46×MICA/B bsAb backbones based on human IgG1 and having enhanced ADCC function and enhanced serum half-life. The sequences here are based on heterodimeric Fc backbone 8 in FIG. 13, although the ADCC variants in FIG. 7 may also be included in any of the other heterodimeric Fc backbones in FIG. 13. ADCC-enhanced Heterodimeric Backbone 1 w/Xtend includes S239D/I332E on both the first and the second heterodimeric Fc chain. ADCC-enhanced Heterodimeric Backbone 2 w/Xtend includes S239D on both the first and the second heterodimeric Fc chain. ADCC-enhanced Heterodimeric Backbone 3 w/Xtend includes 1332E on both the first and the second heterodimeric Fc chain. ADCC-enhanced Heterodimeric Backbone 4 w/Xtend includes S239D/I332E on the second heterodimeric Fc chain. ADCC-enhanced Heterodimeric Backbone 5 w/Xtend includes S239D on the second heterodimeric Fc chain.

ADCC-enhanced Heterodimeric Backbone 6 w/Xtend includes 1332E on the second heterodimeric Fc chain. ADCC-enhanced Heterodimeric Backbone 7 w/Xtend includes S239D/I332E on the first heterodimeric Fc chain. ADCC-enhanced Heterodimeric Backbone 8 w/Xtend includes S239D on the first heterodimeric Fc chain. ADCC-enhanced Heterodimeric Backbone 9 w/Xtend includes 1332E on the first heterodimeric Fc chain. ADCC-enhanced Heterodimeric Backbone 10 w/Xtend includes S239D/I332E on the first heterodimeric Fc chain and S239D on the second heterodimeric Fc chain. ADCC-enhanced Heterodimeric Backbone 11 w/Xtend includes S239D/I332E on the first heterodimeric Fc chain and 1332E on the second heterodimeric Fc chain. ADCC-enhanced Heterodimeric Backbone 12 w/Xtend includes S239D on the first heterodimeric Fc chain and S239D/I332E on the second heterodimeric Fc chain. ADCC-enhanced Heterodimeric Backbone 13 w/Xtend includes 1332E on the first heterodimeric Fc chain and S239D/I332E on the second heterodimeric Fc chain. ADCC-enhanced Heterodimeric Backbone 14 w/Xtend includes S239D on the first heterodimeric Fc chain and 1332E on the second heterodimeric Fc chain. ADCC-enhanced Heterodimeric Backbone 15 w/Xtend includes 1332E on the first heterodimeric Fc chain and S239D on the second heterodimeric Fc chain.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1. That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

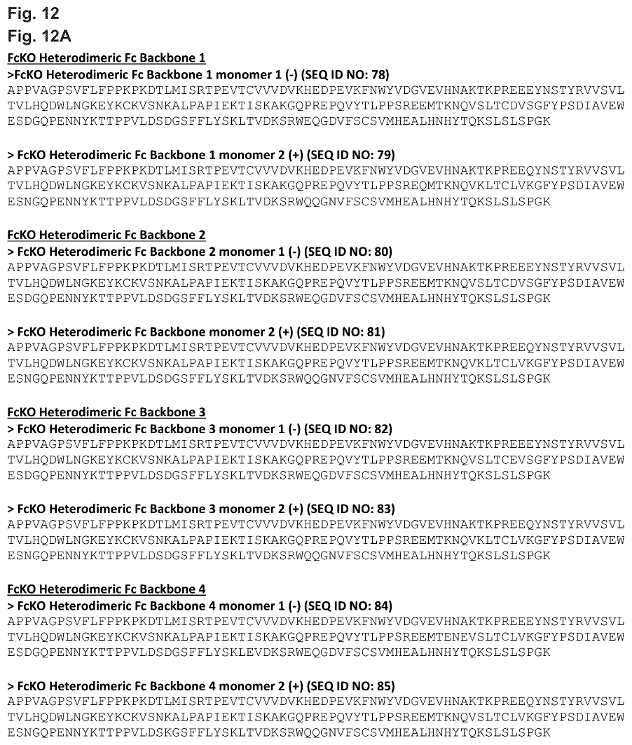

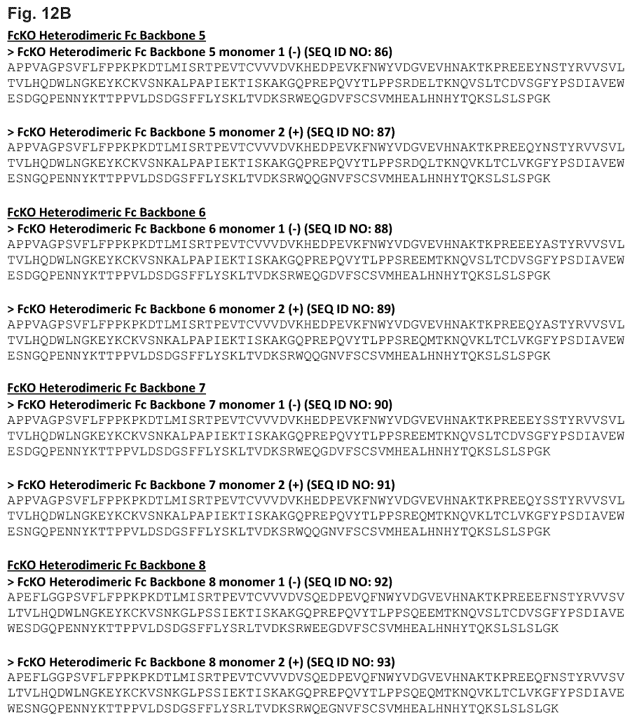

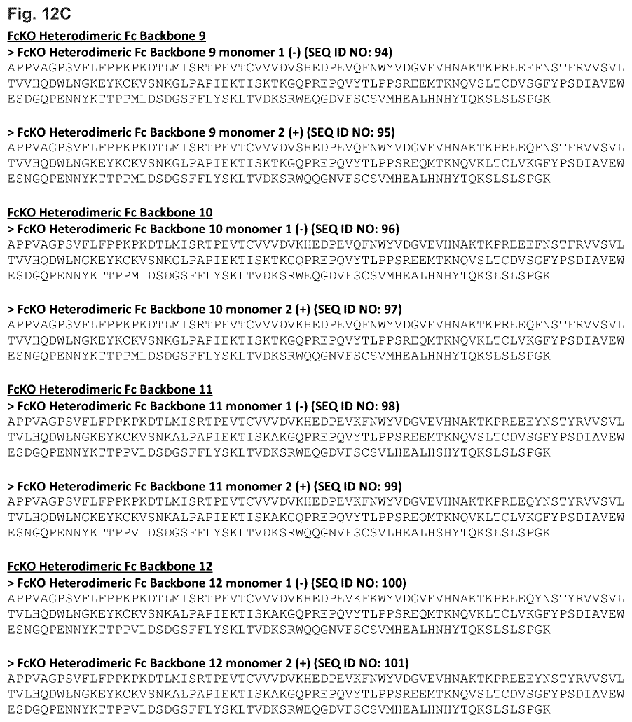

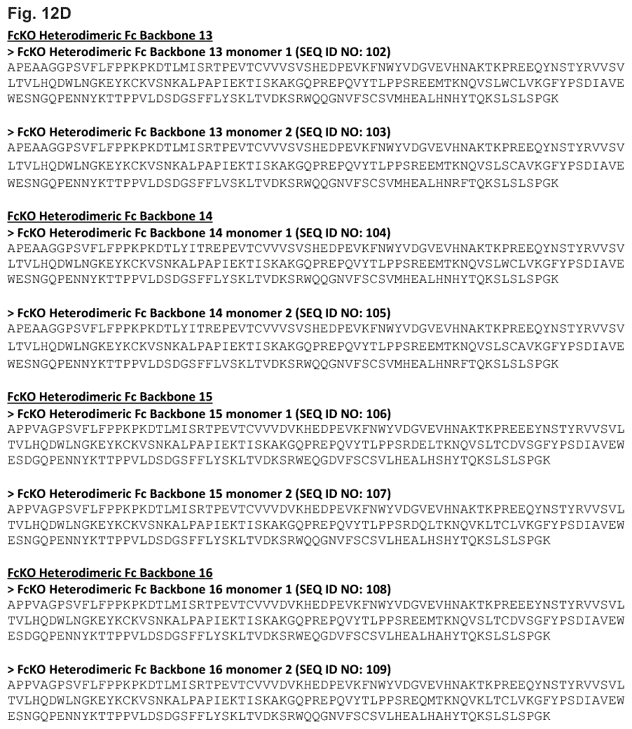

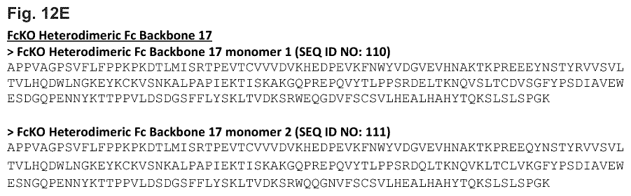

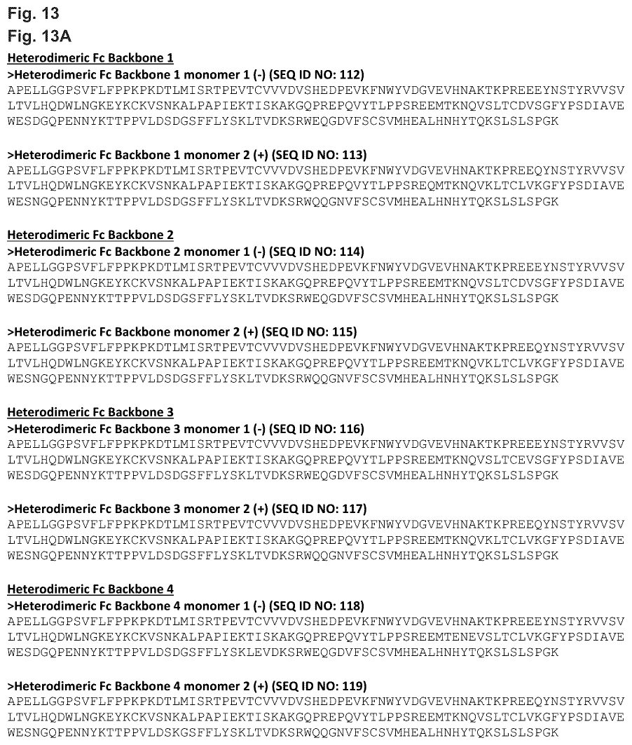

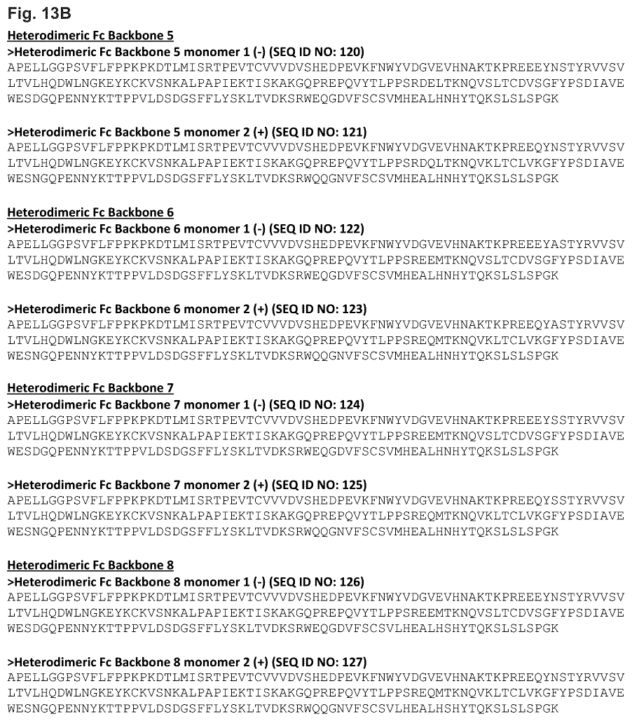

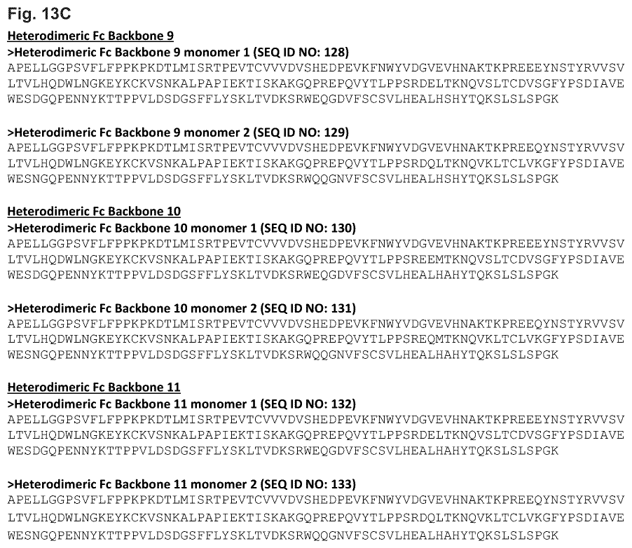

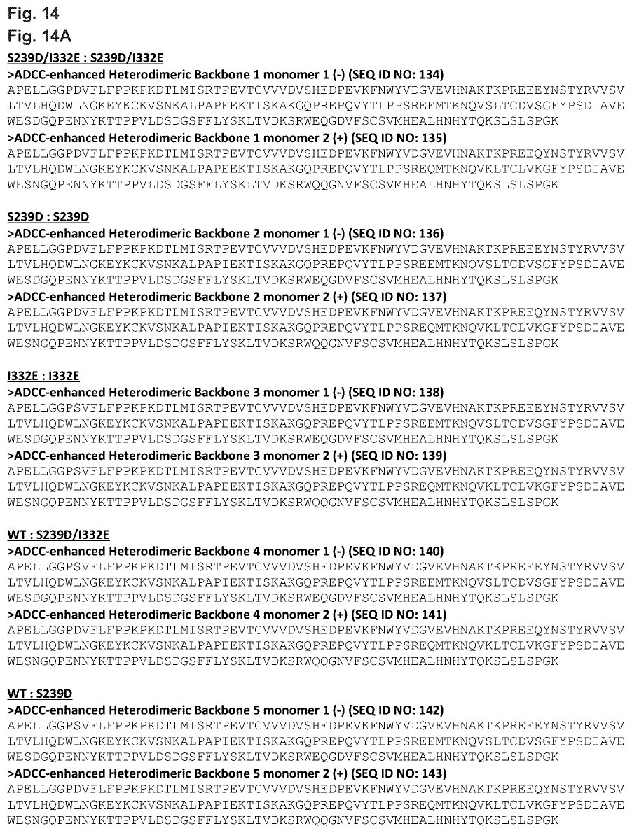

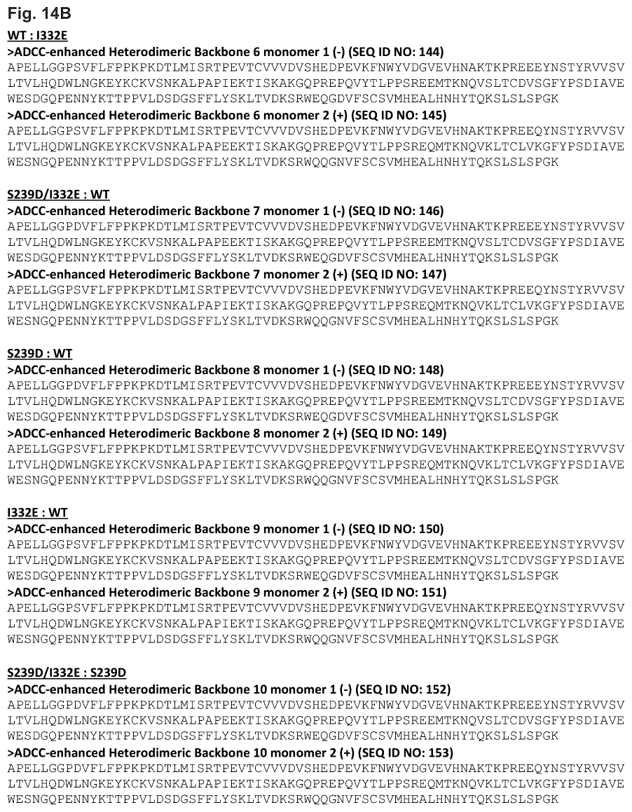

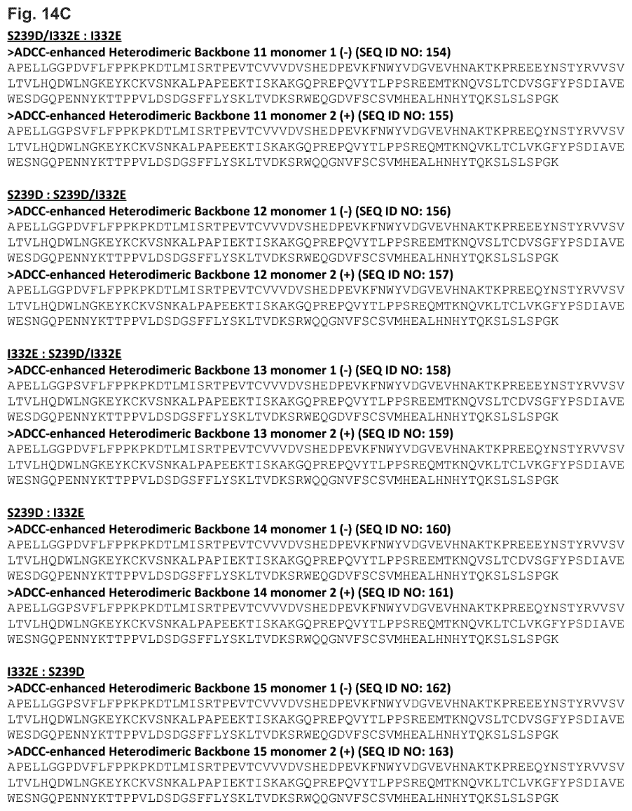

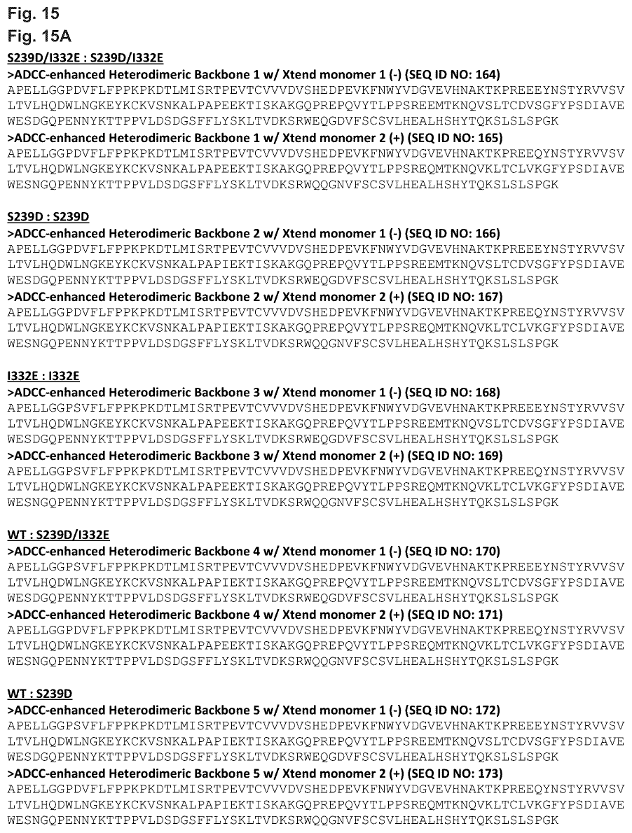

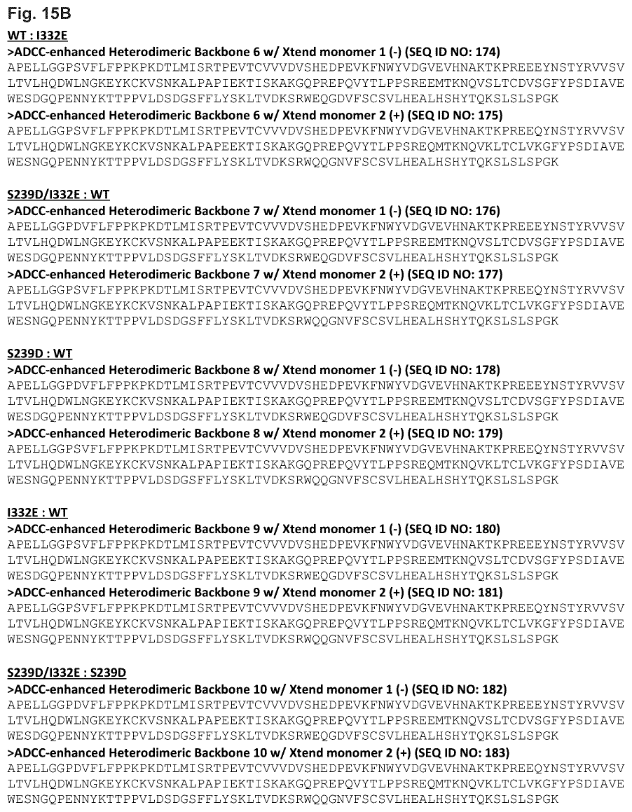

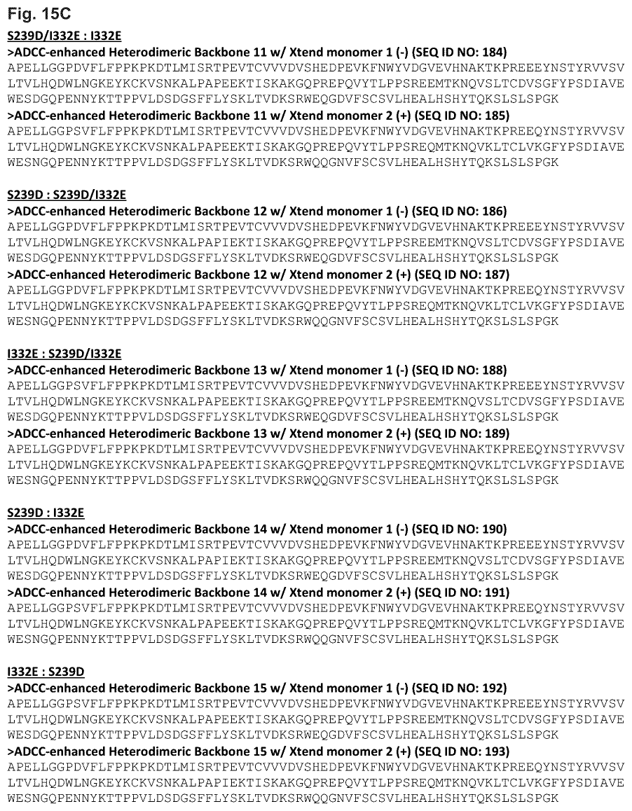

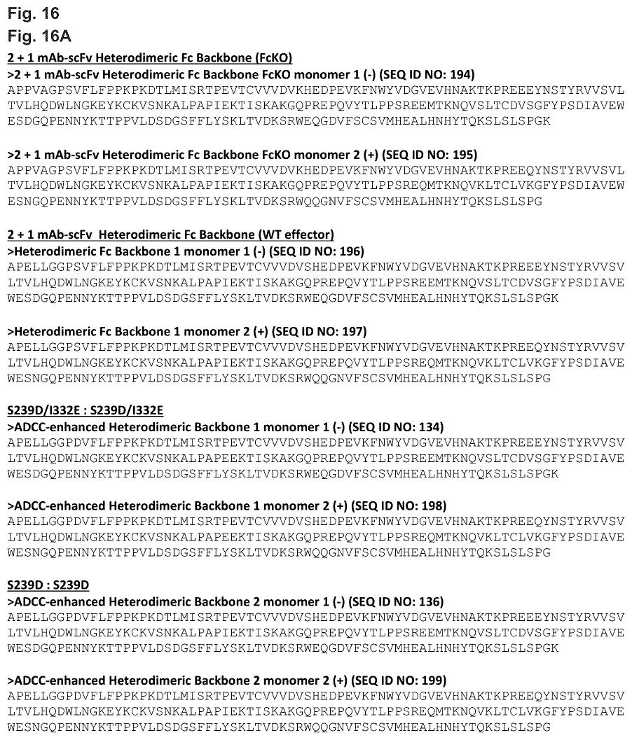

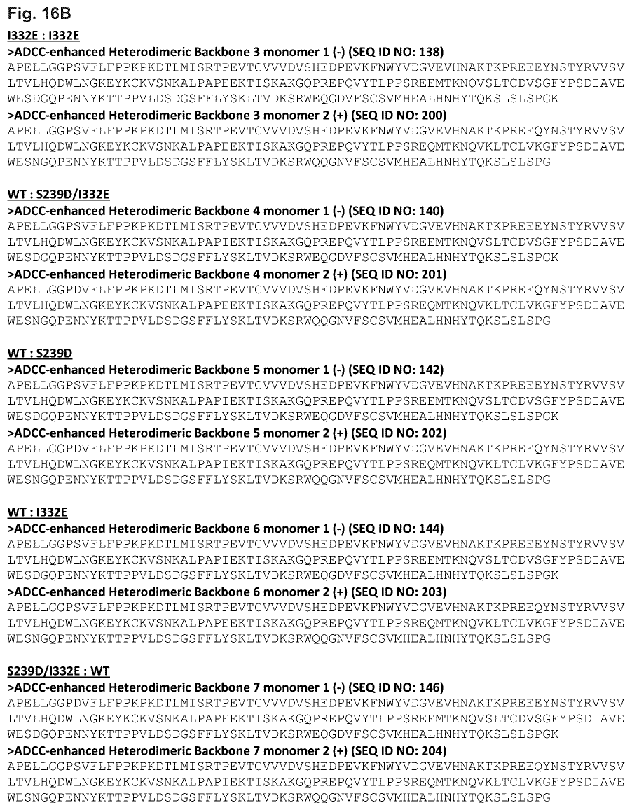

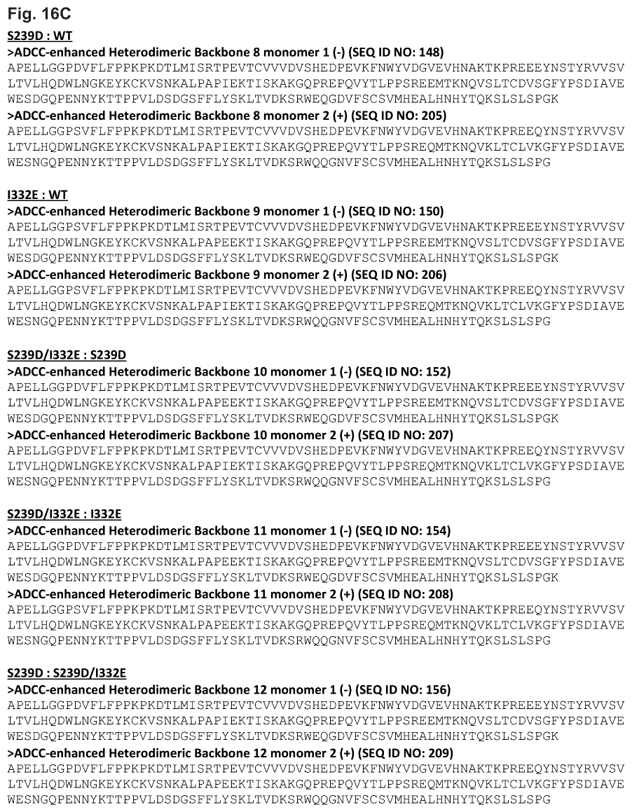

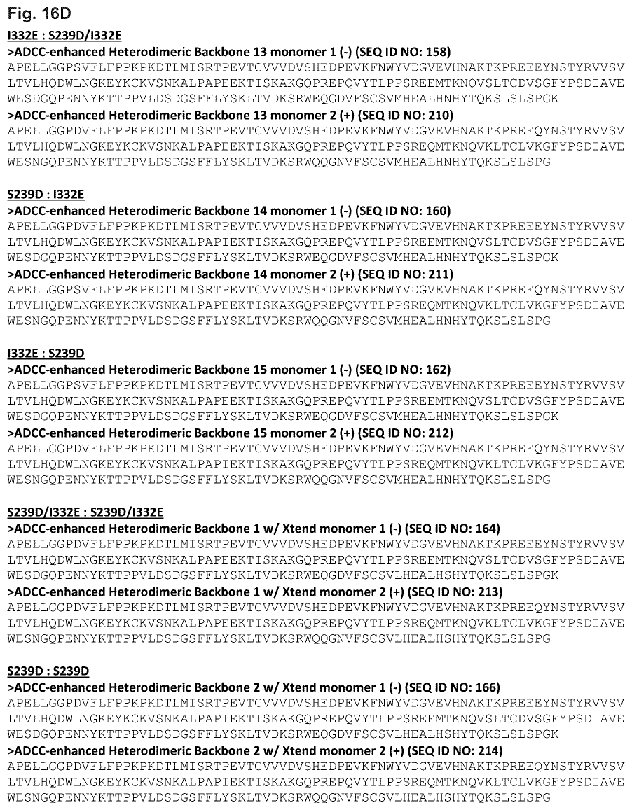

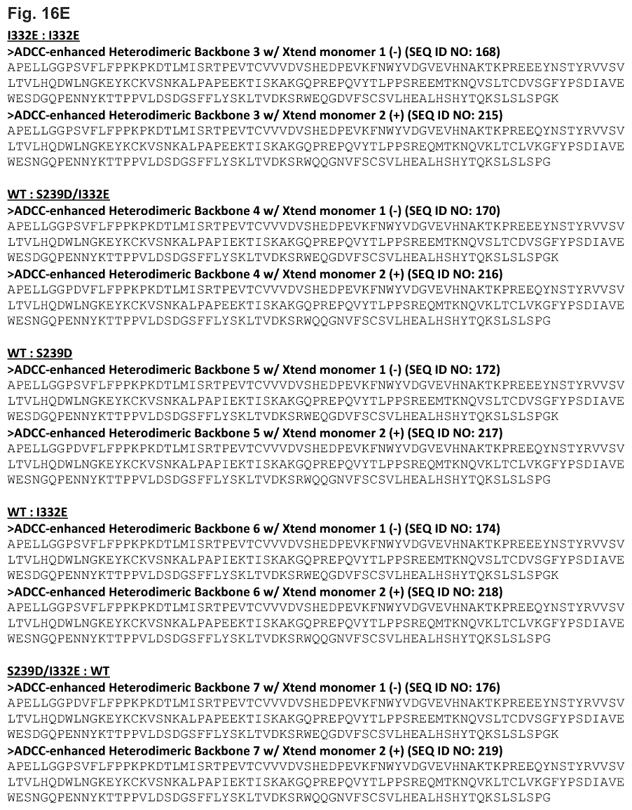

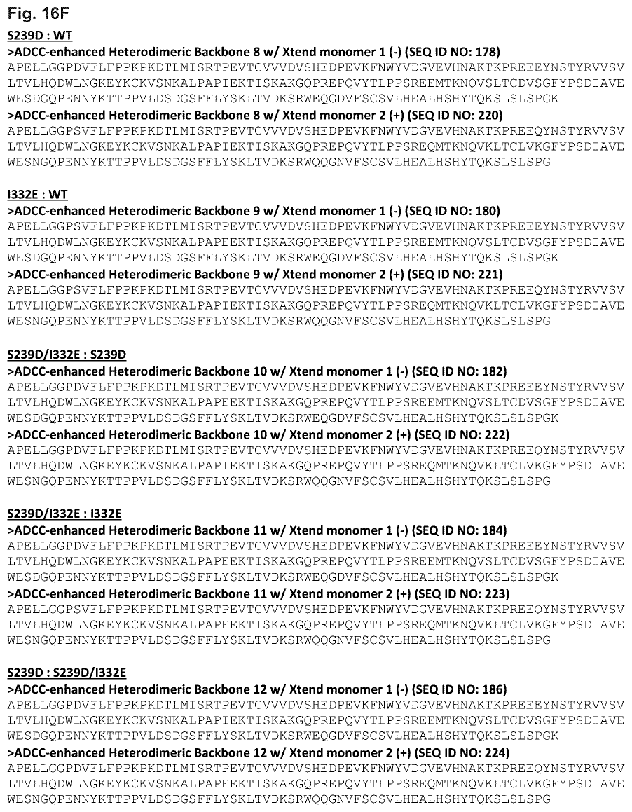

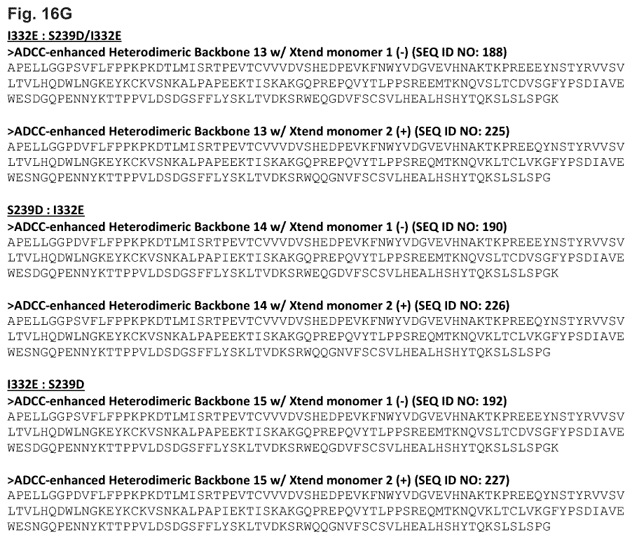

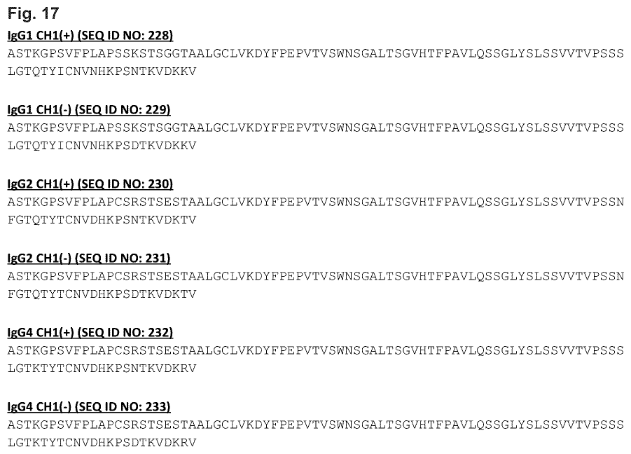

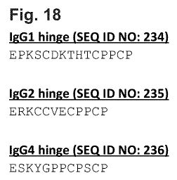

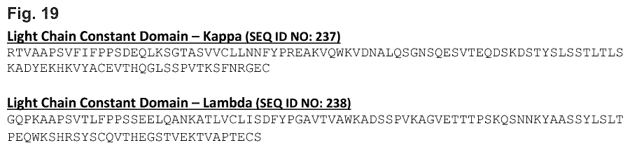

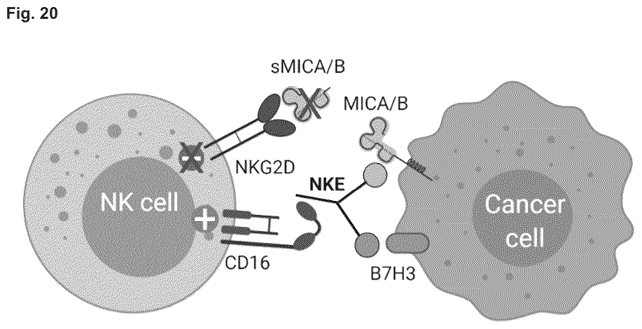

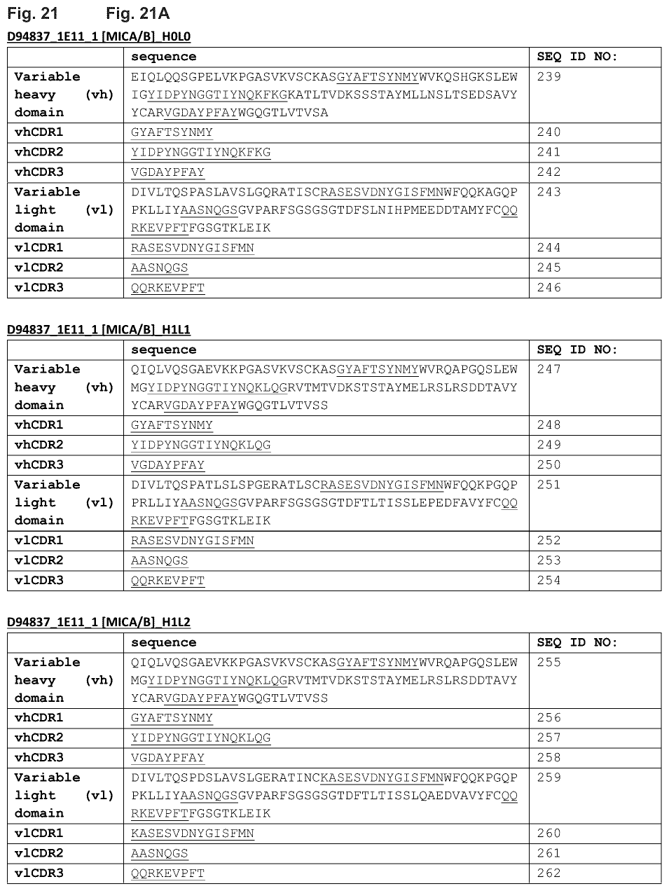

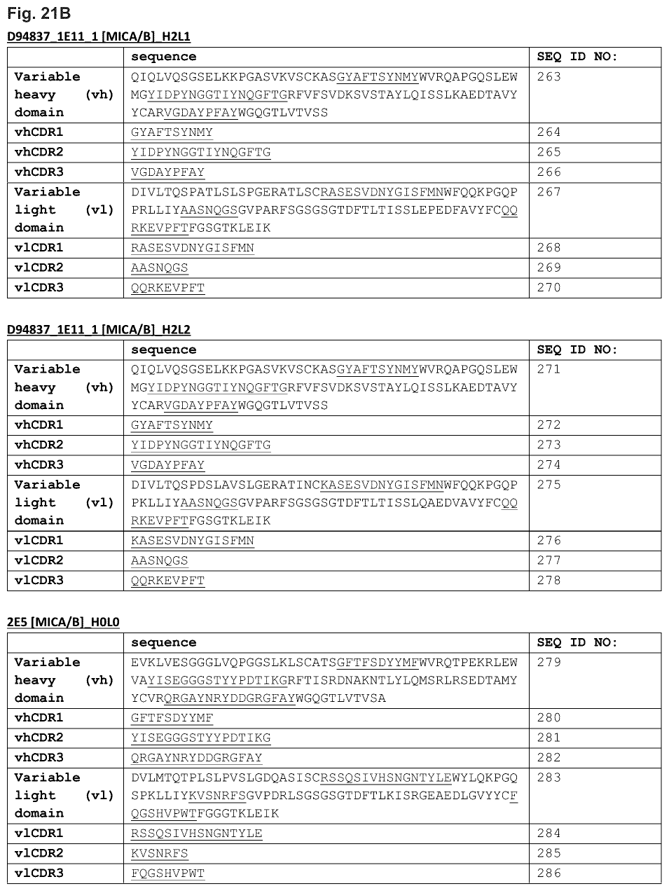

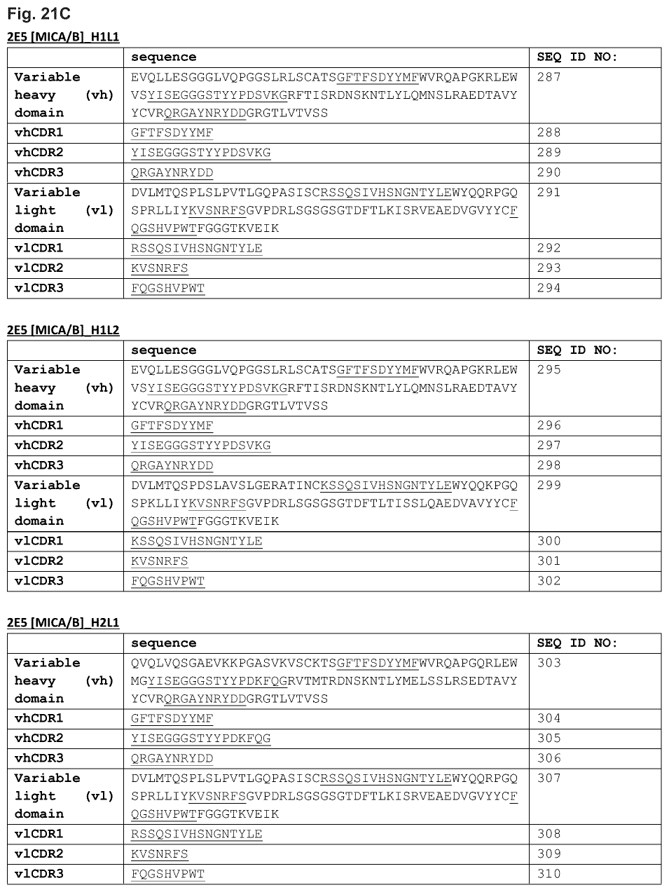

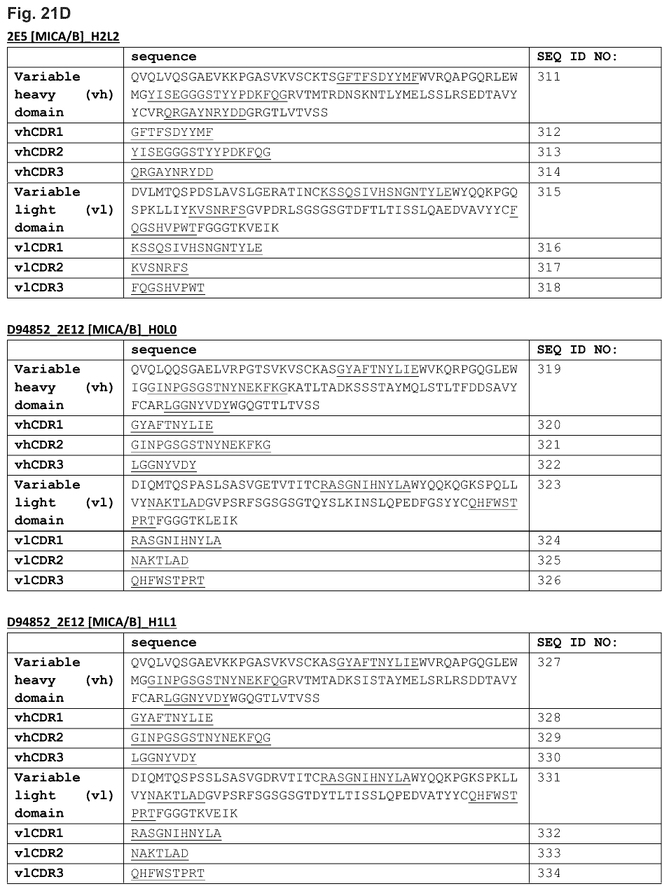

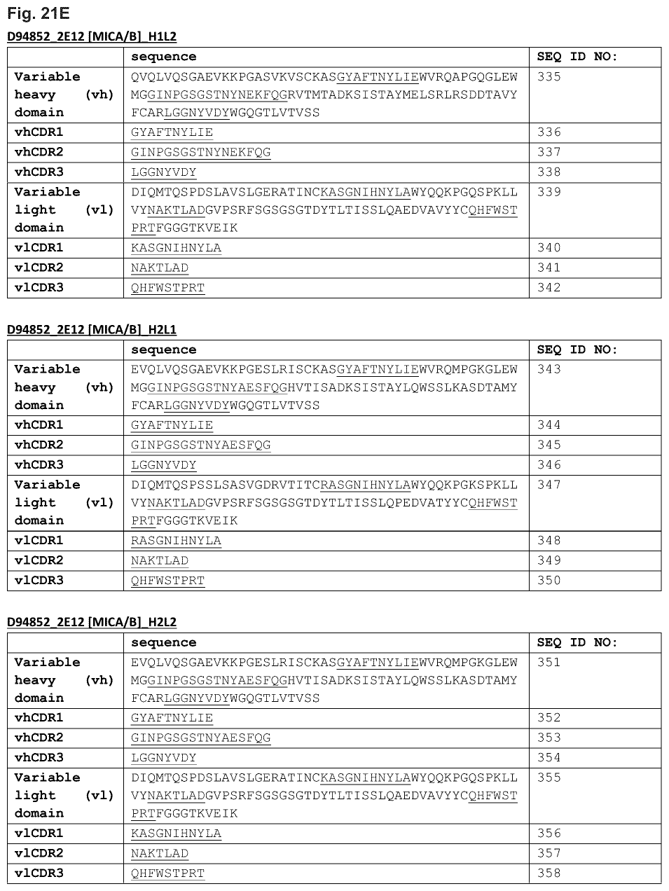

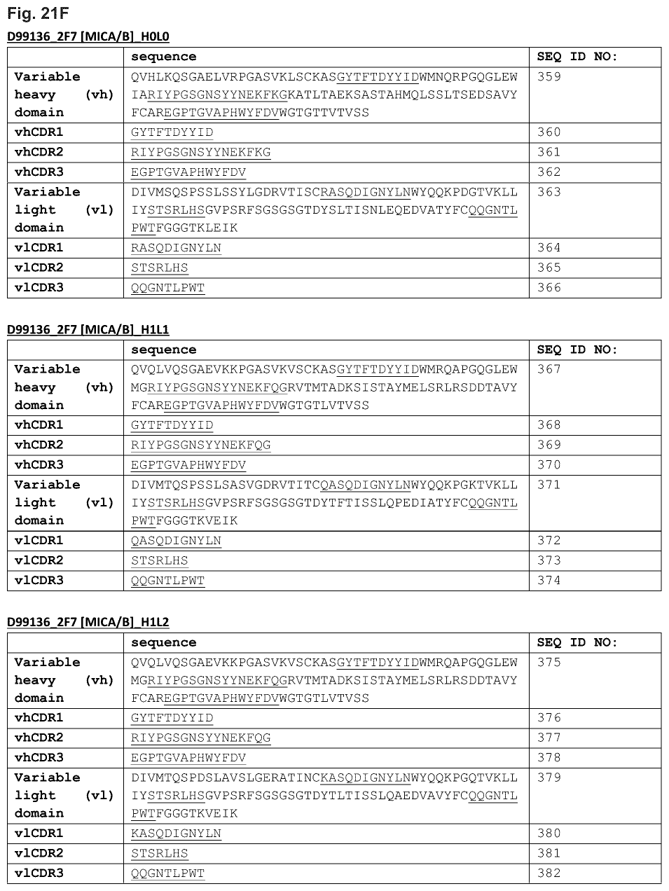

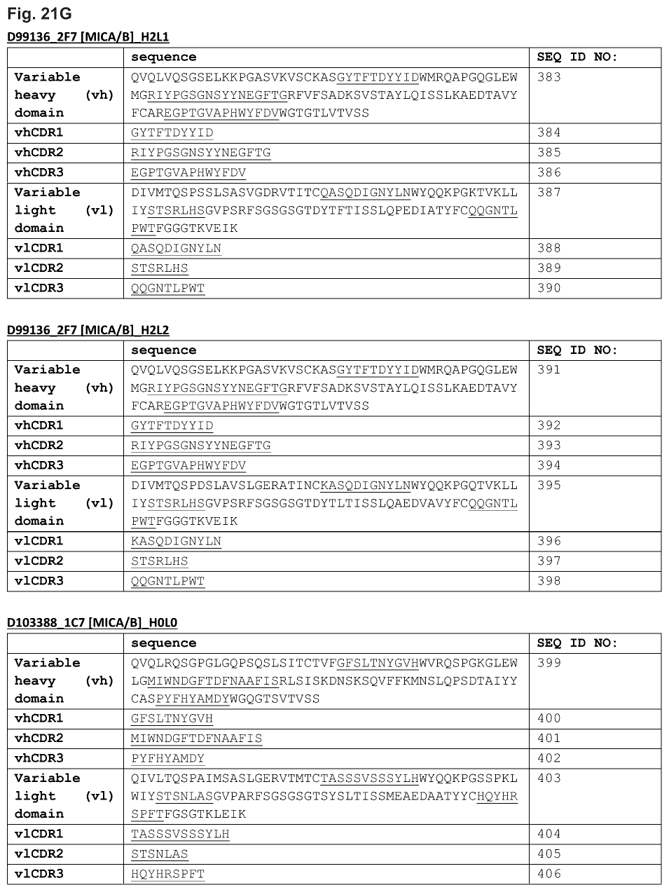

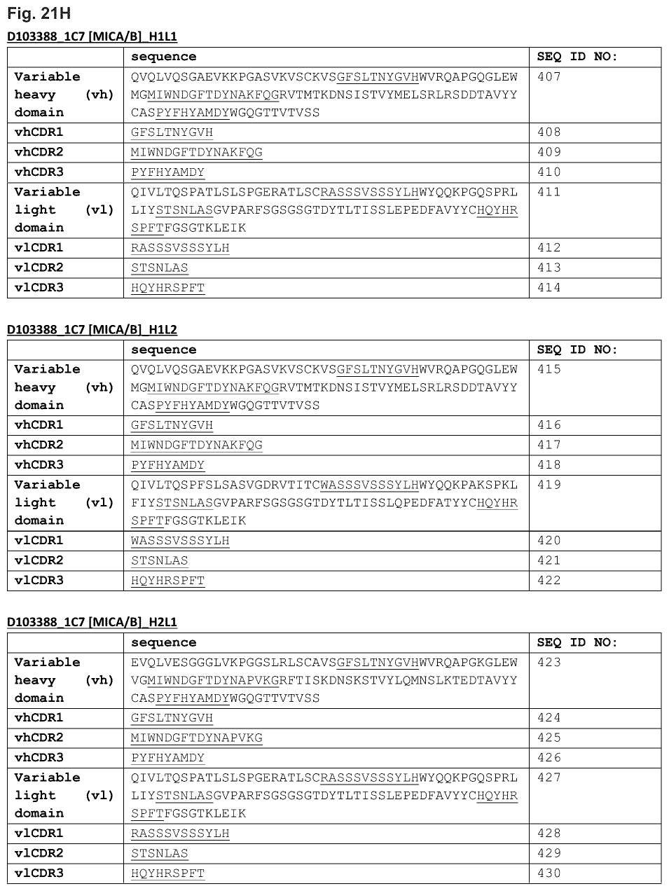

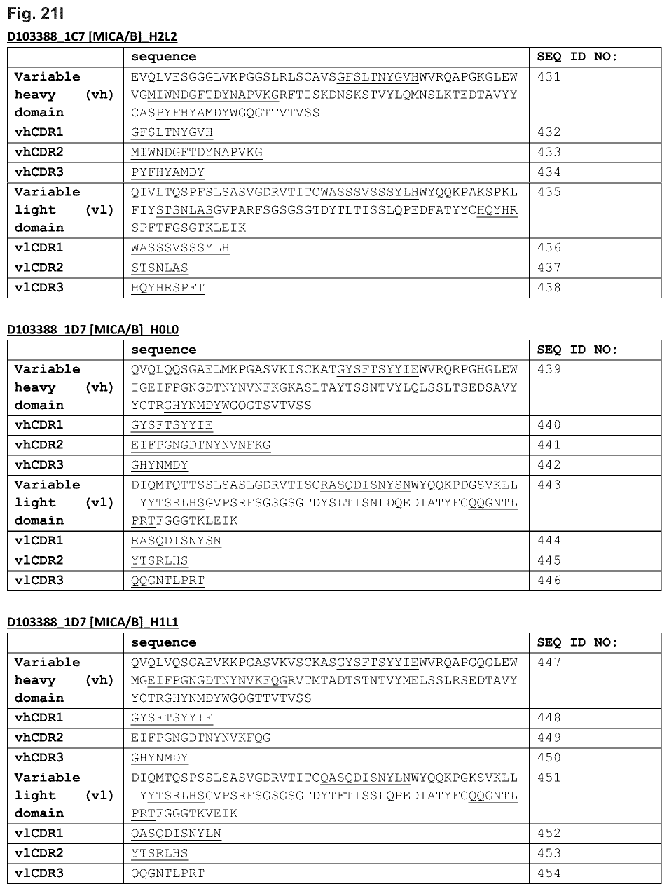

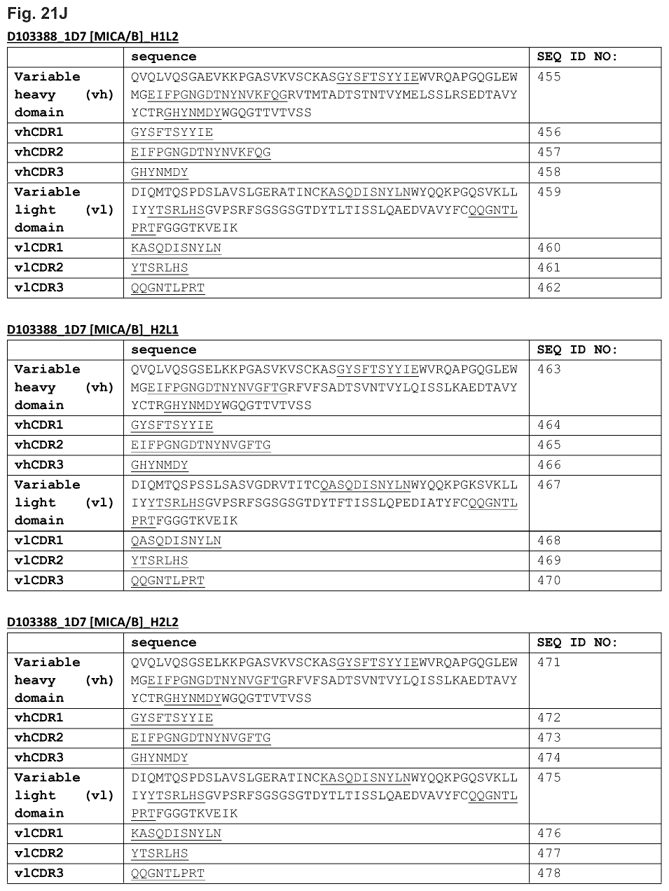

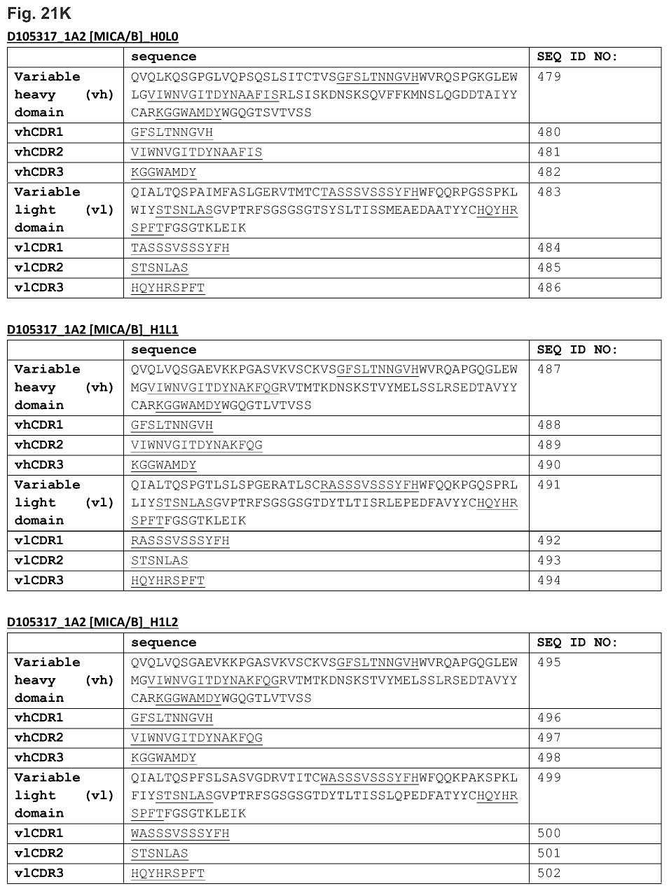

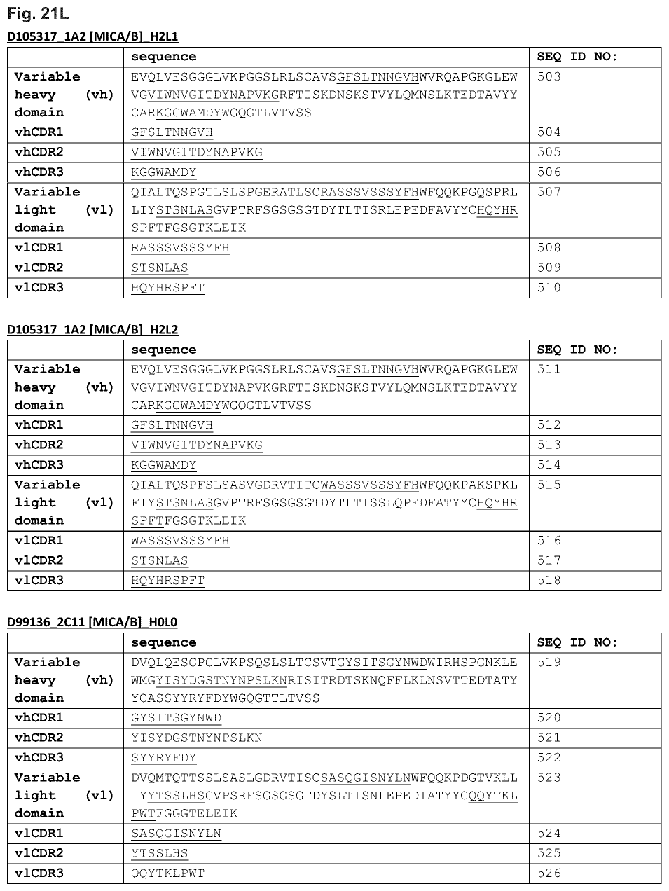

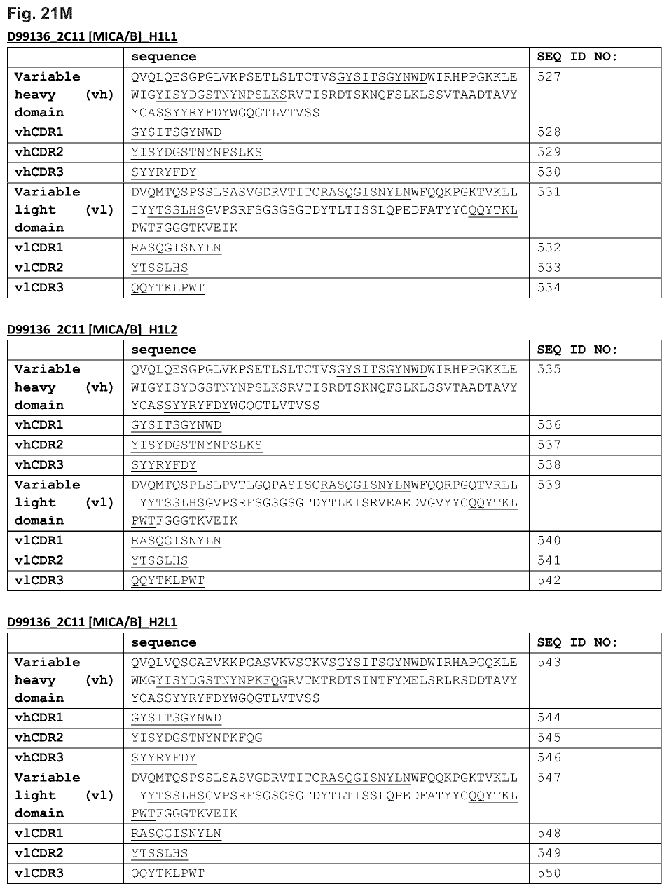

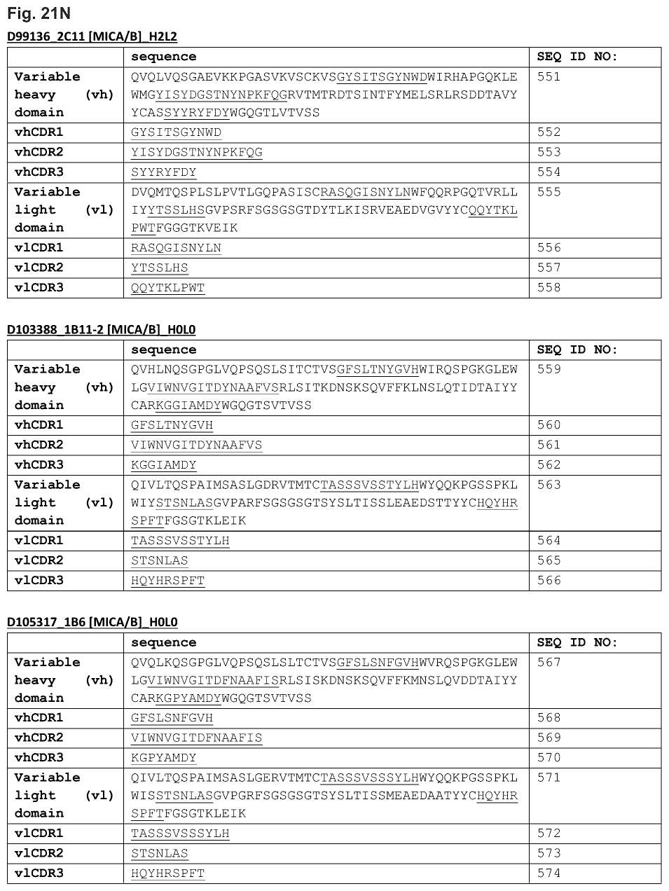

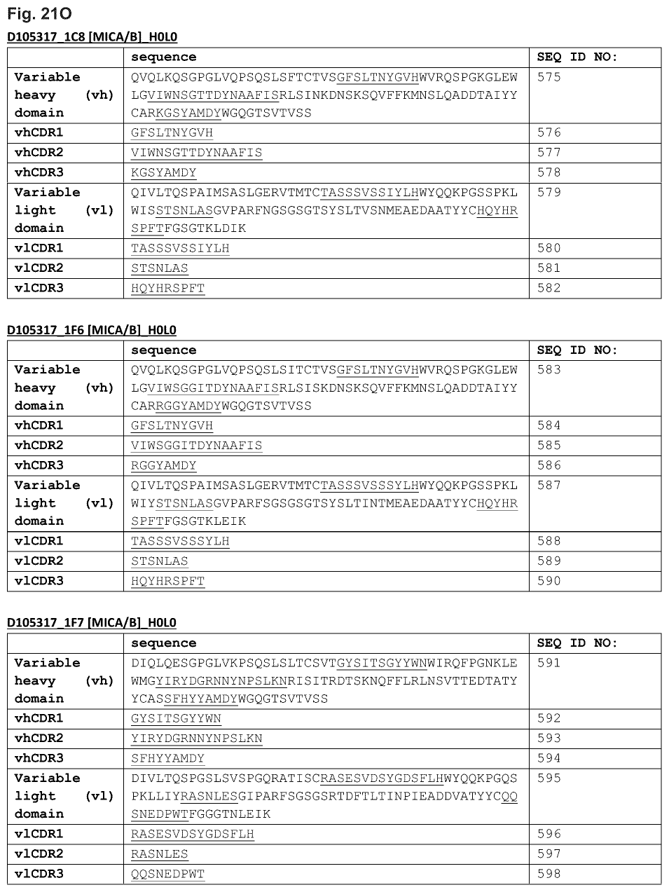

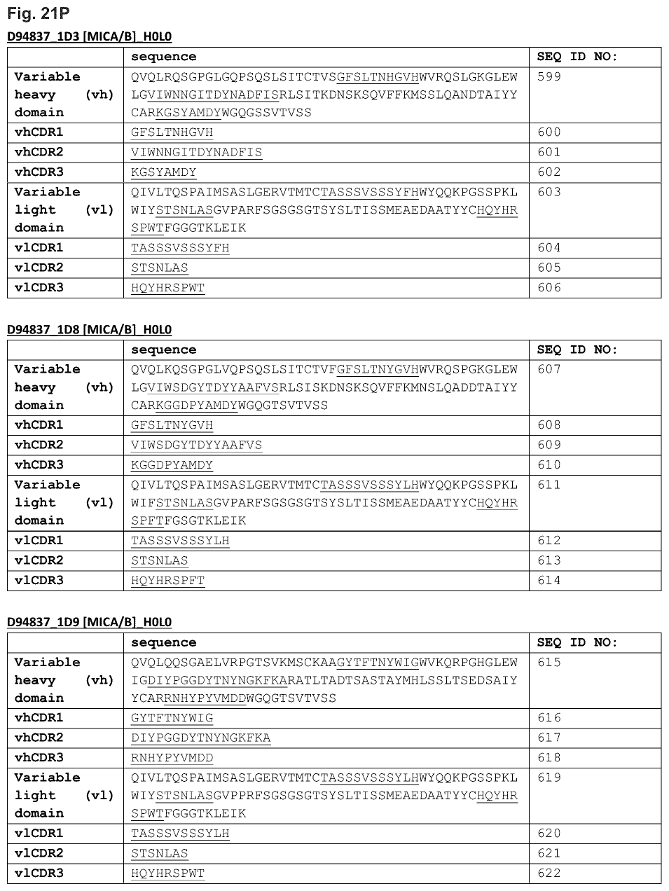

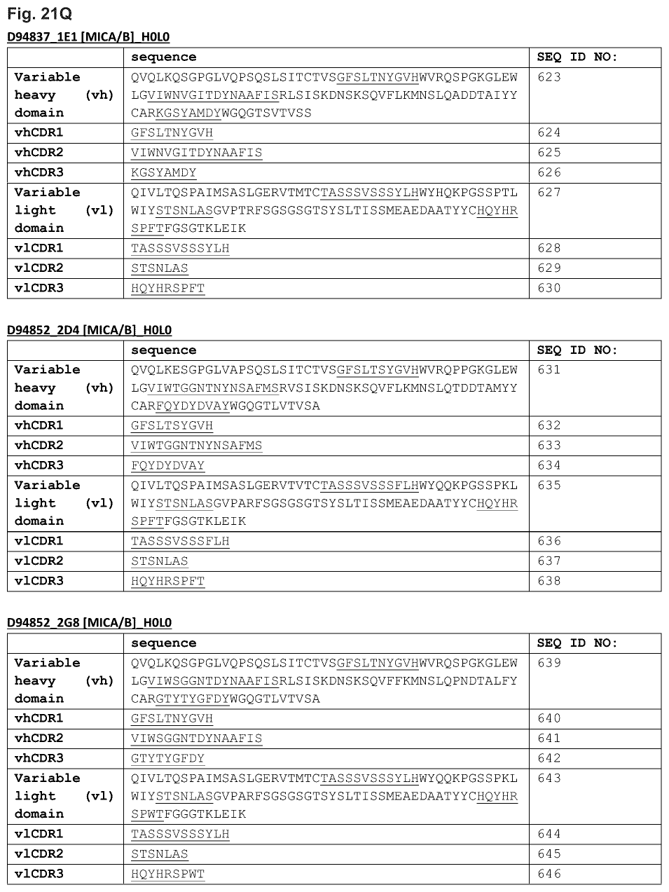

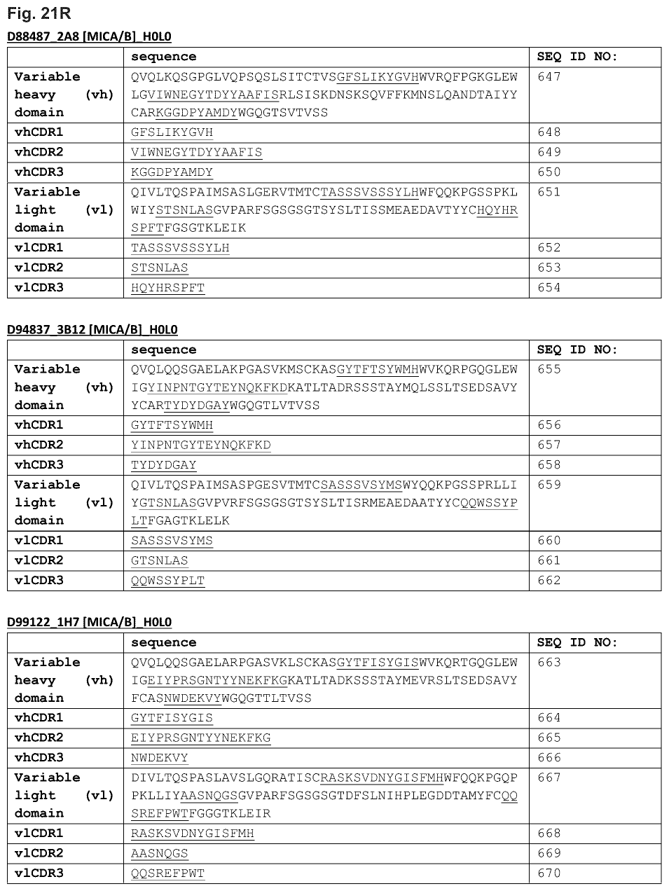

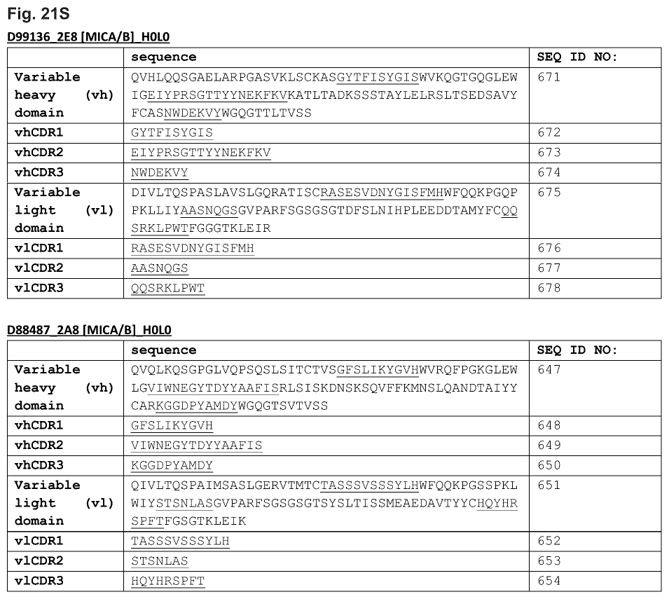

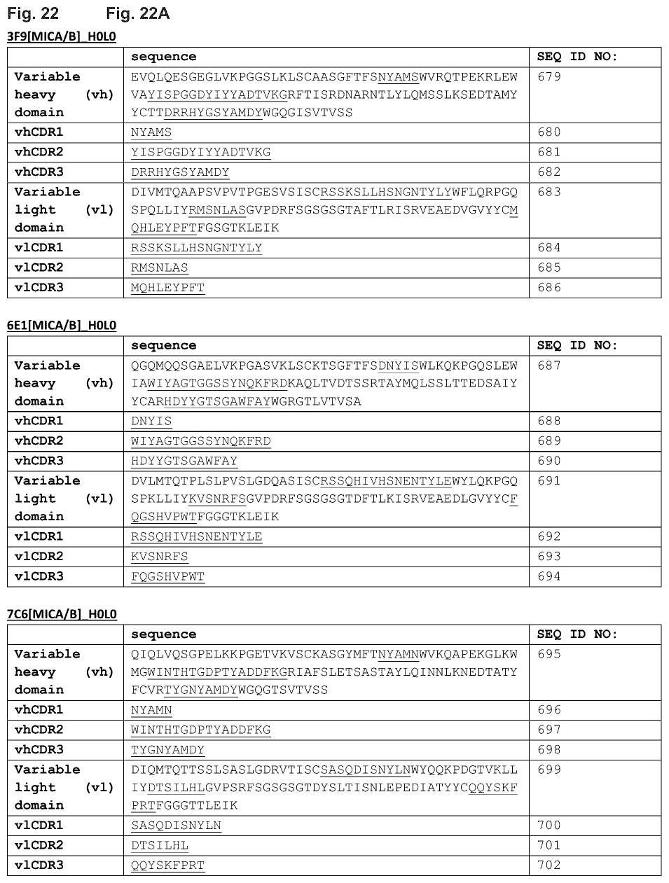

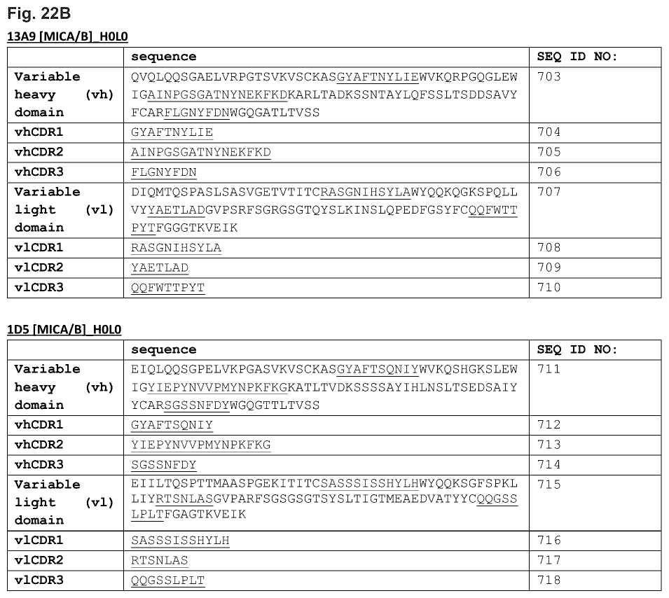

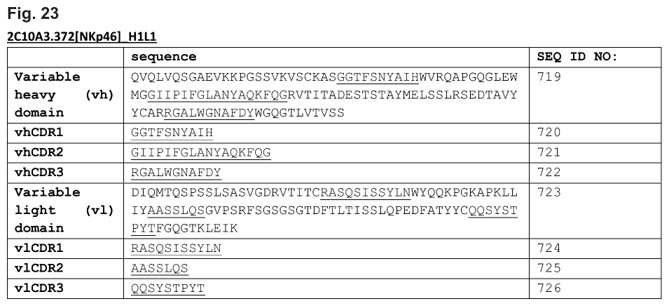

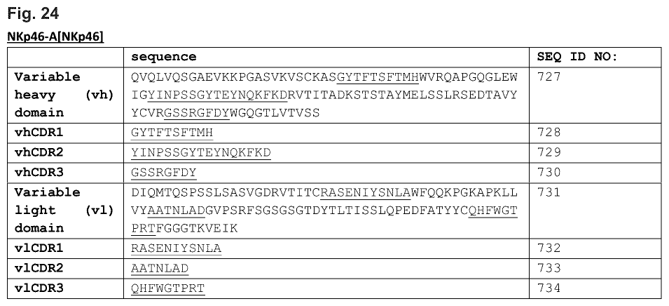

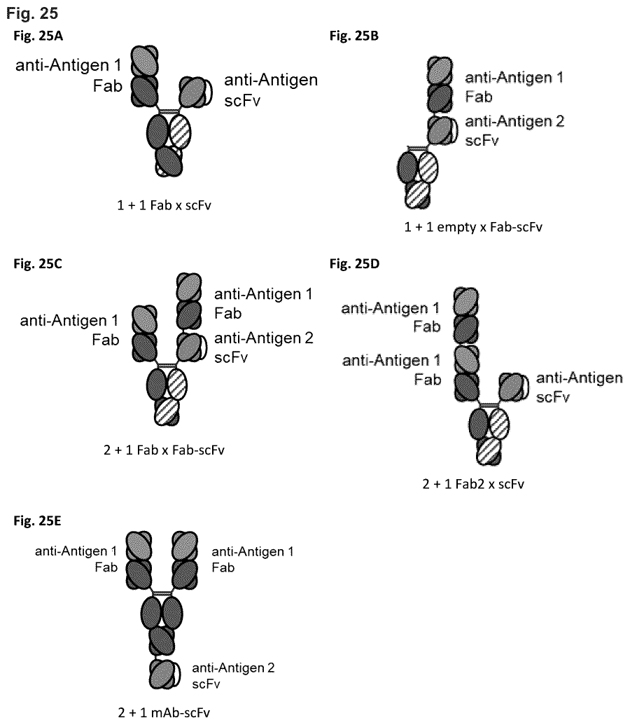

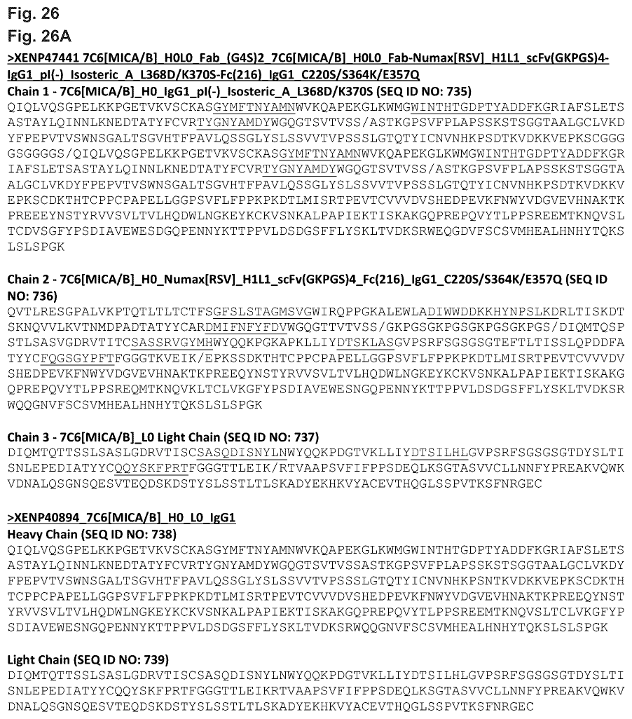

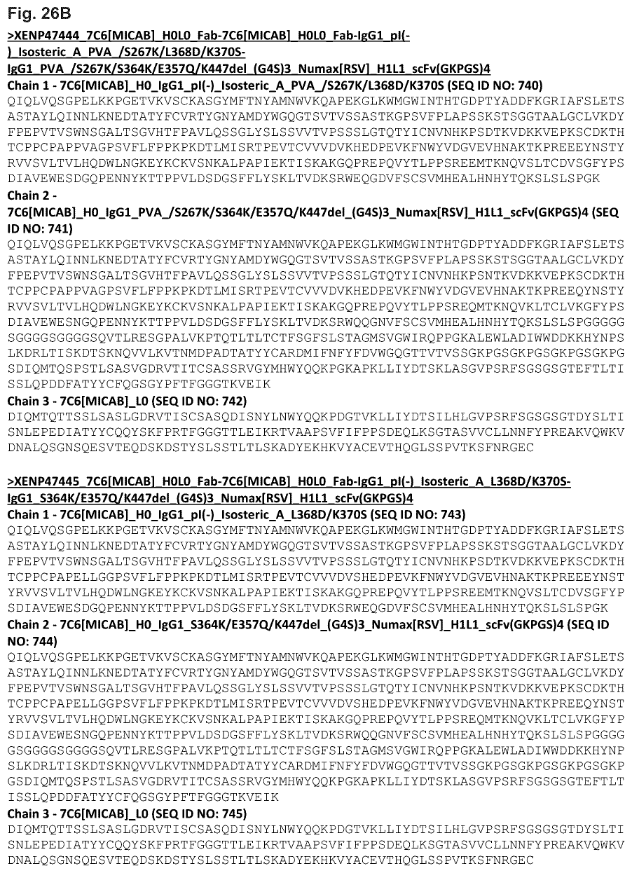

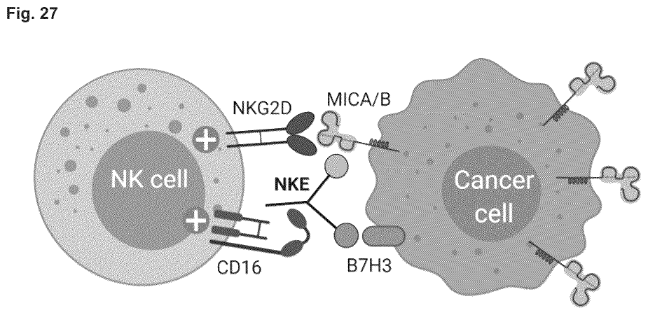

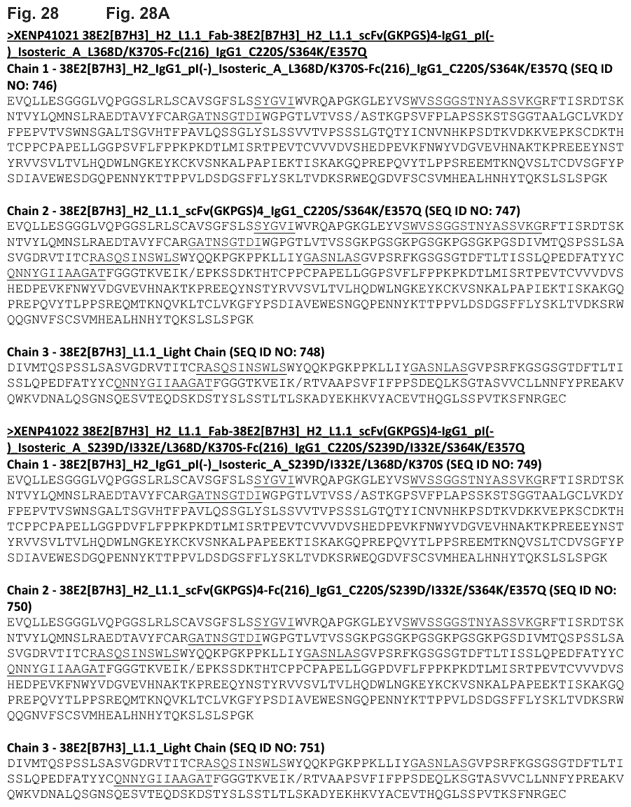

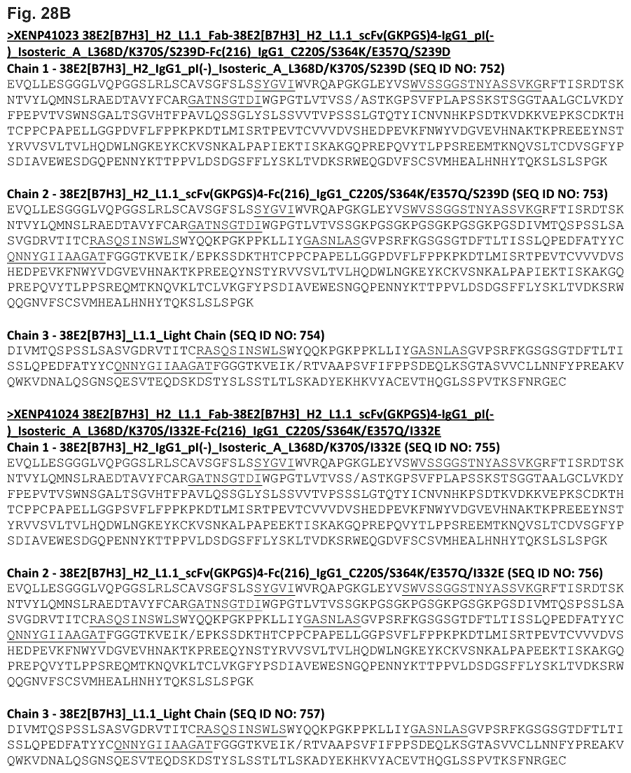

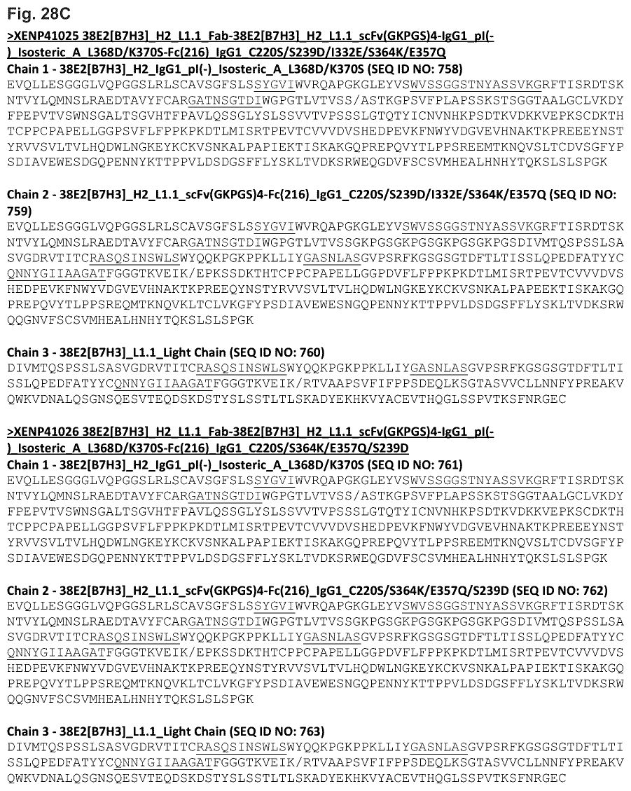

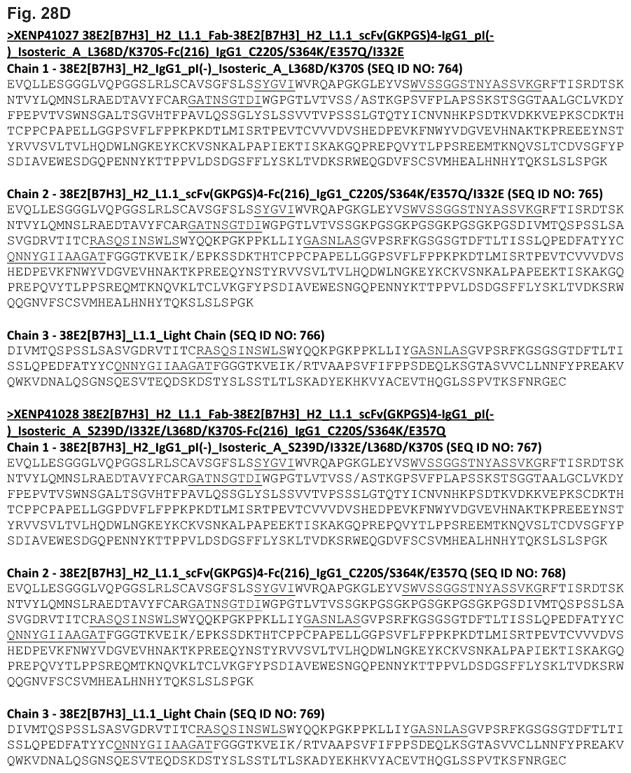

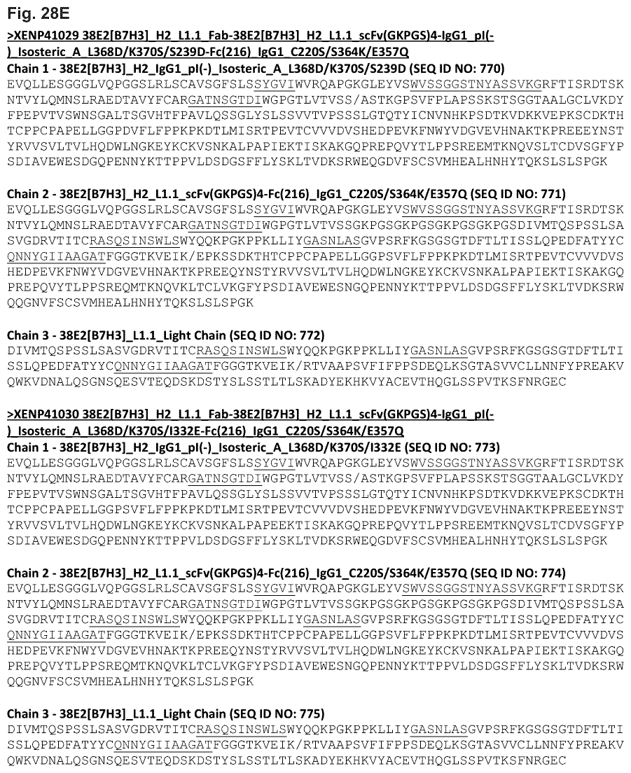

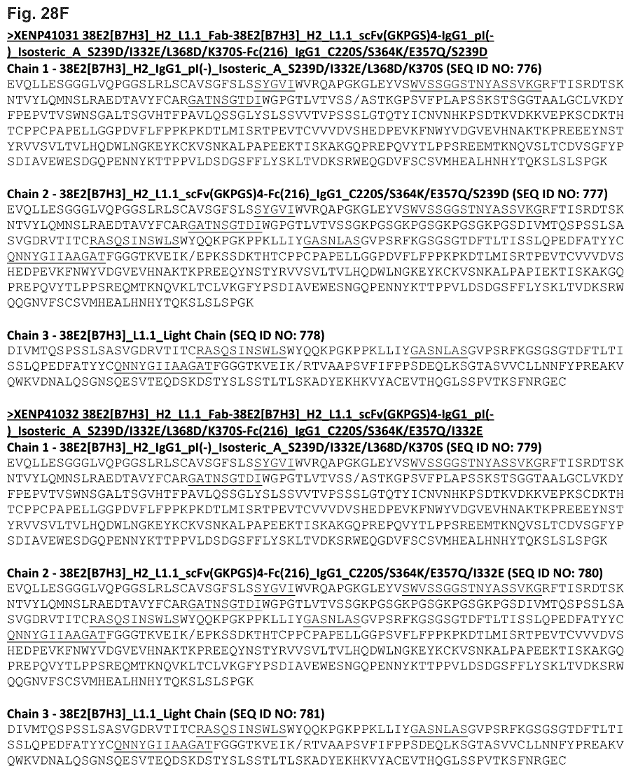

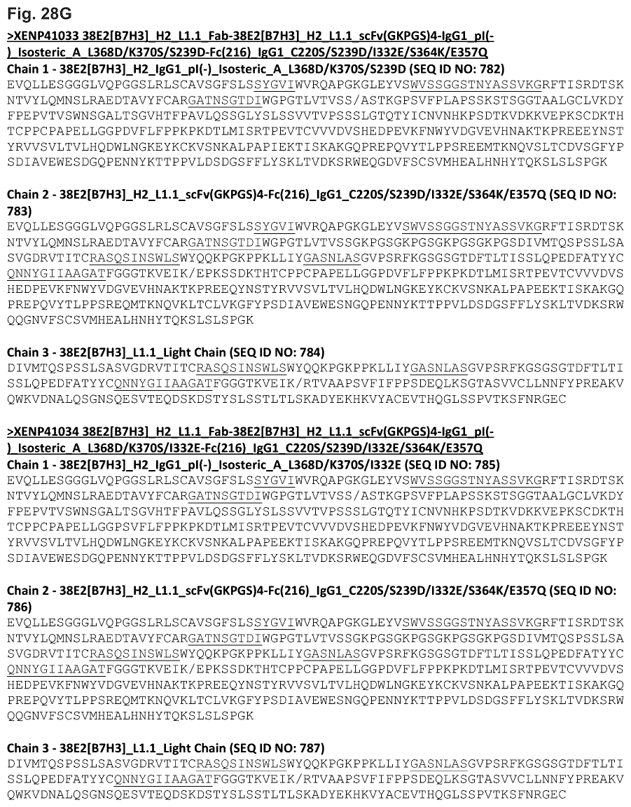

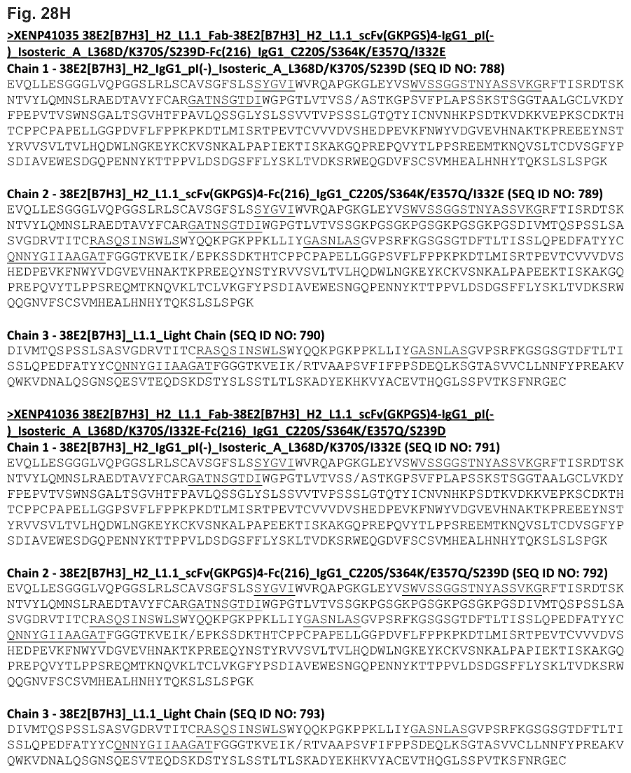

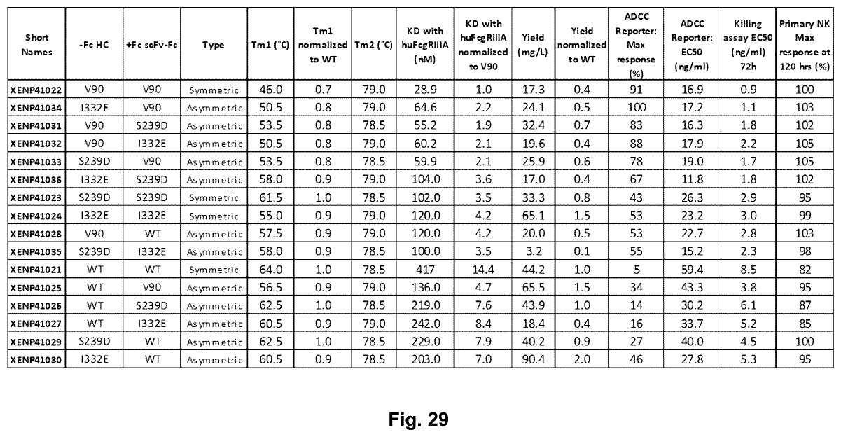

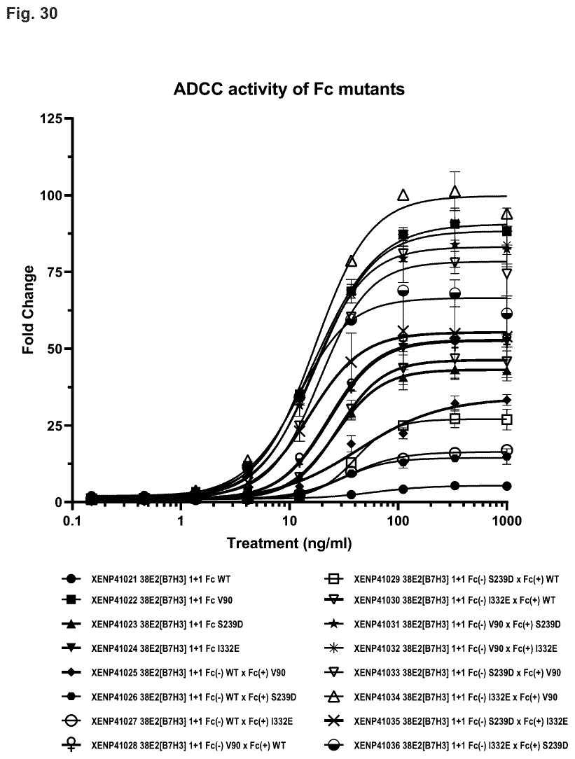

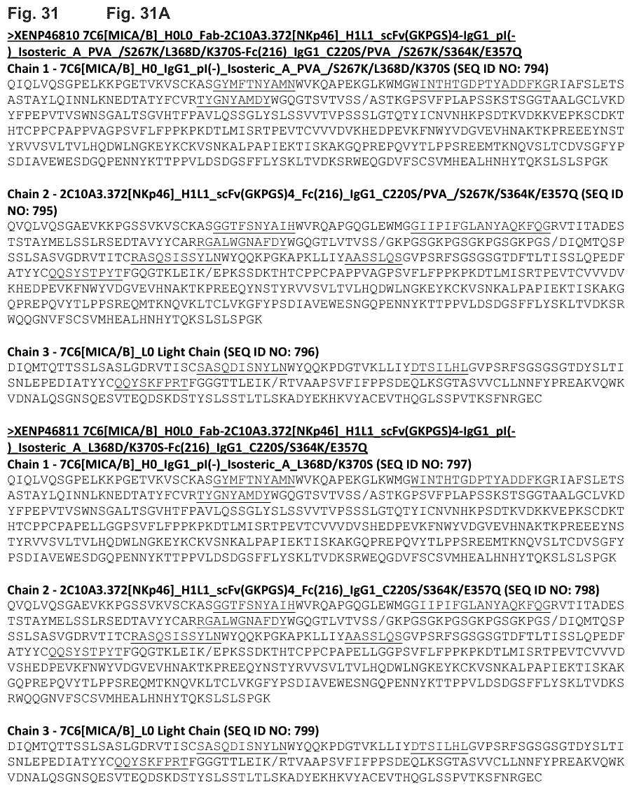

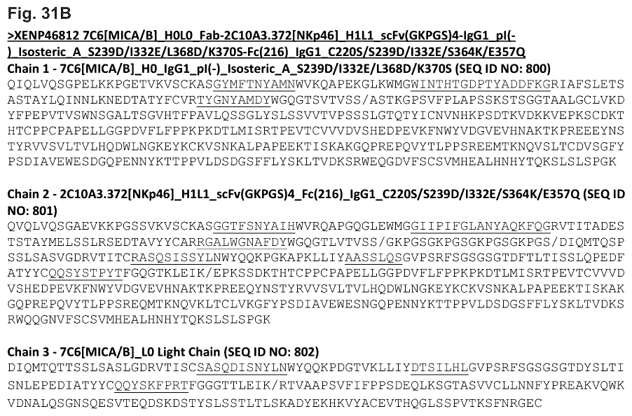

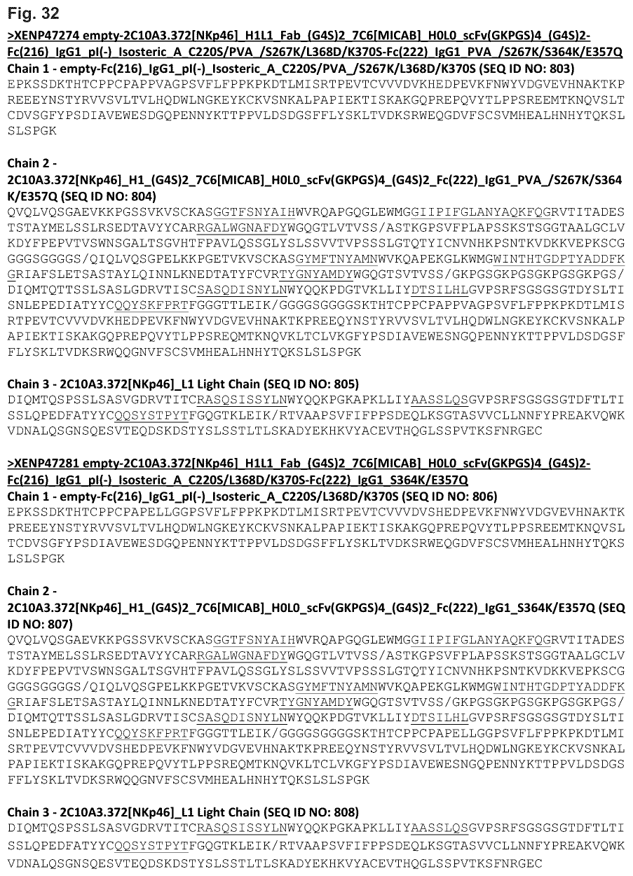

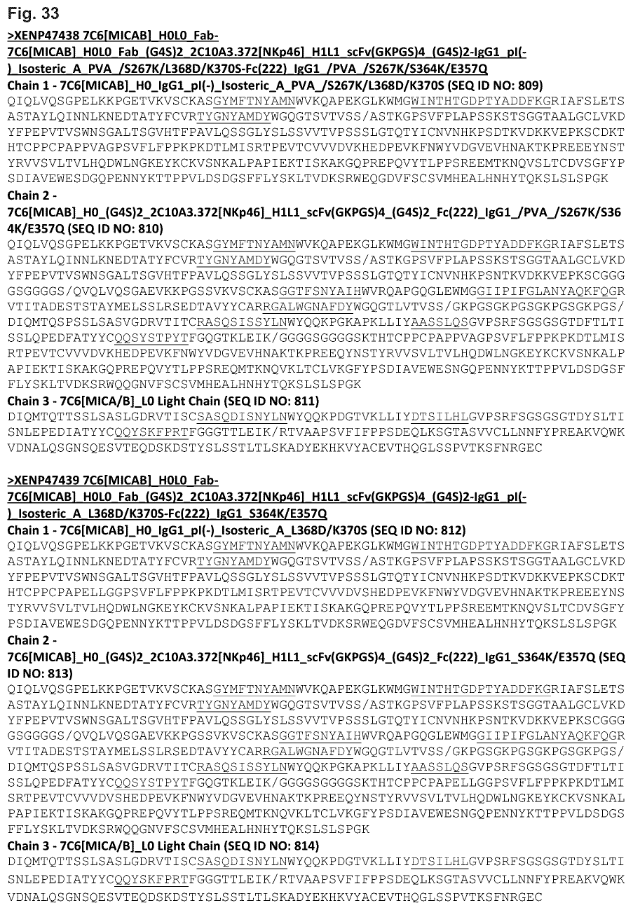

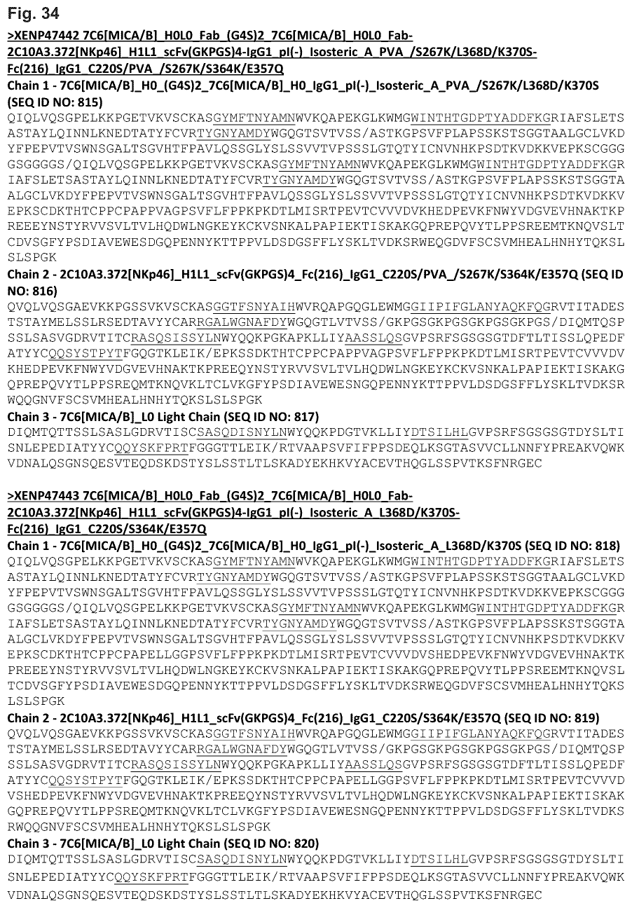

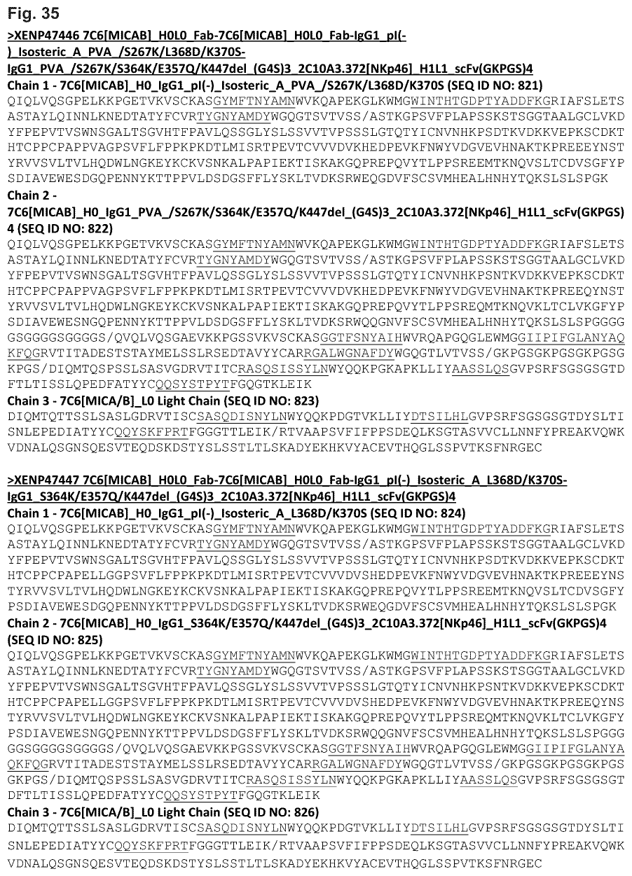

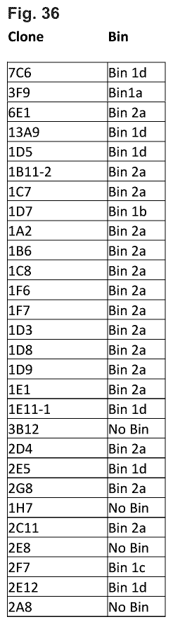

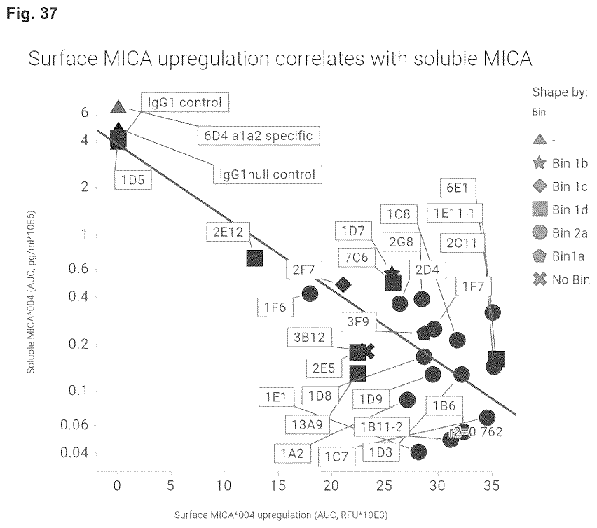

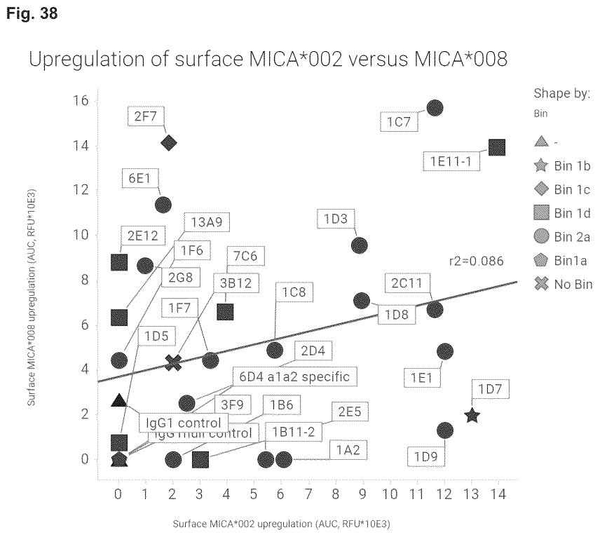

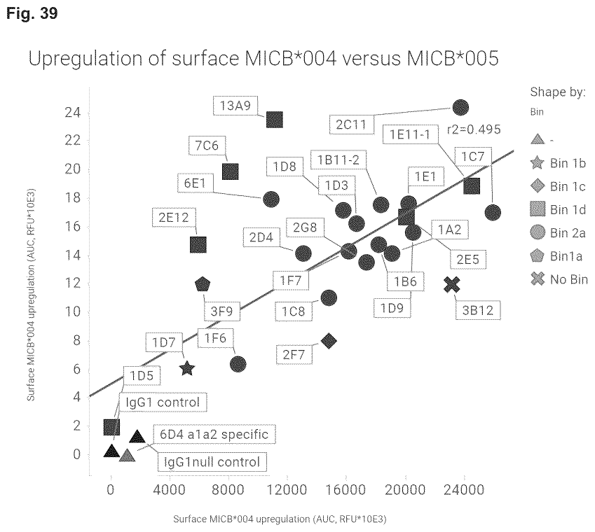

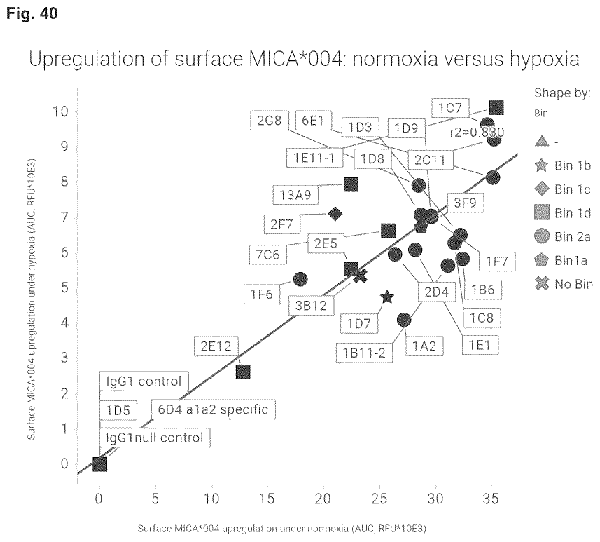

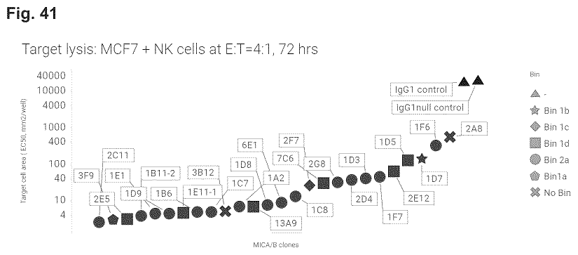

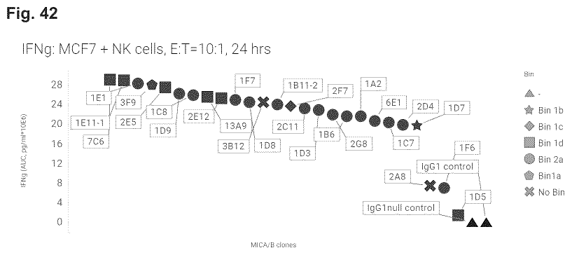

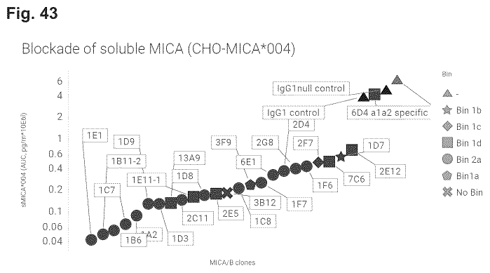

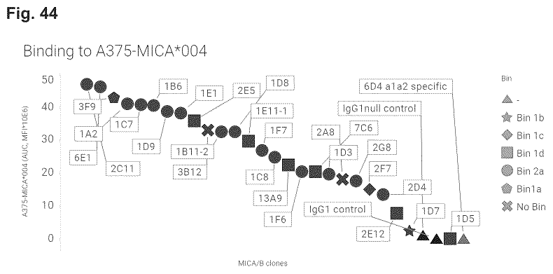

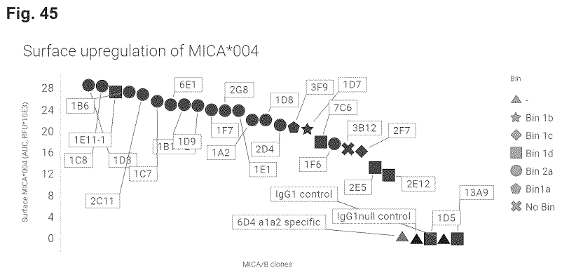

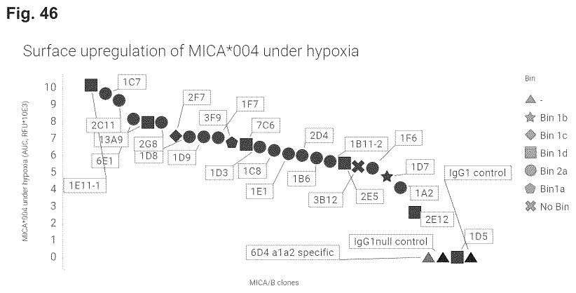

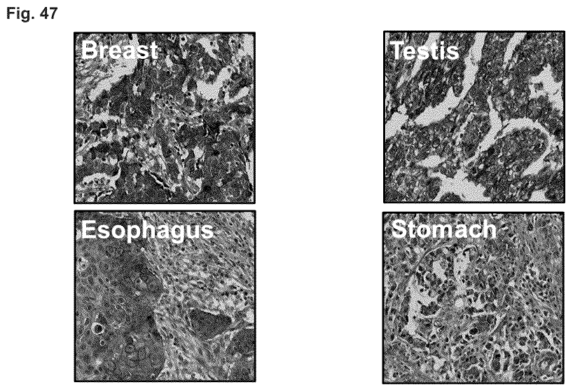

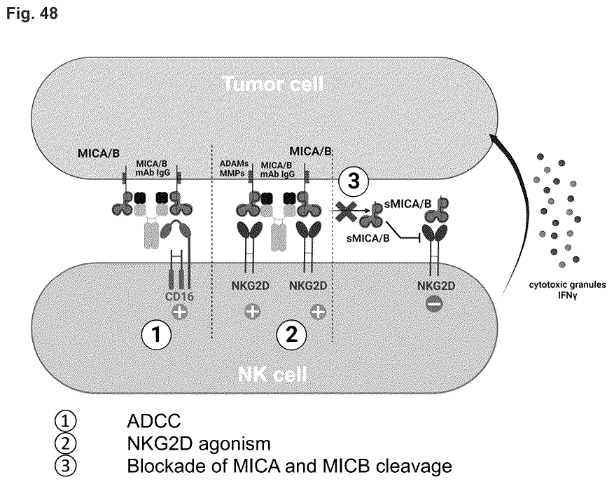

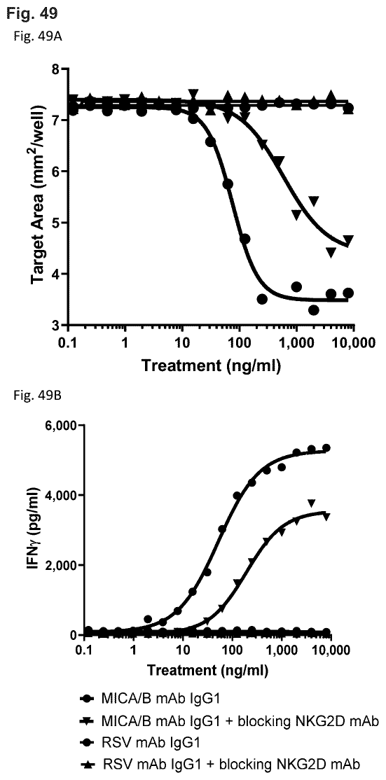

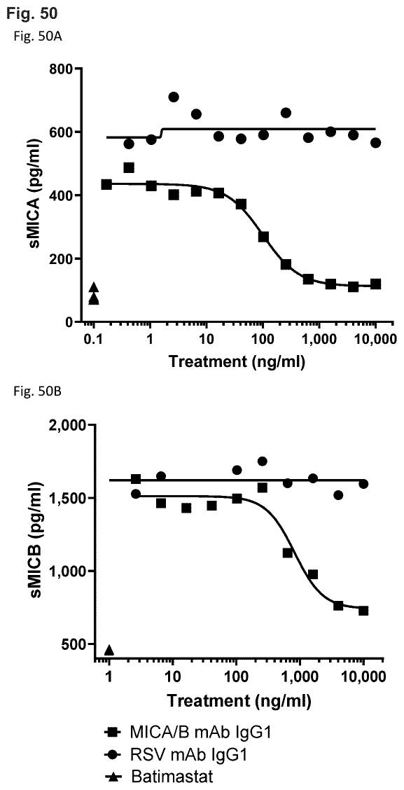

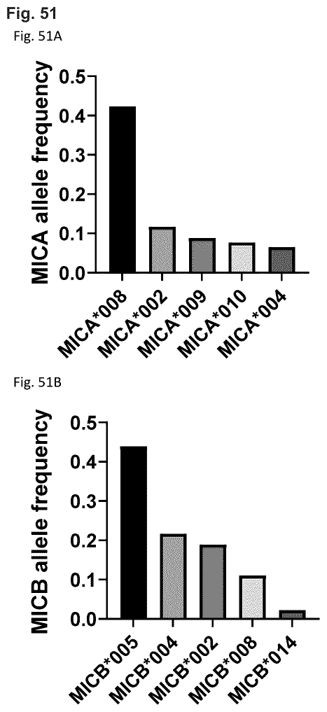

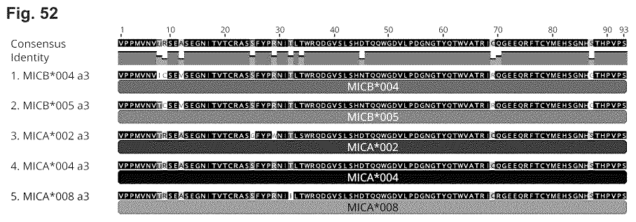

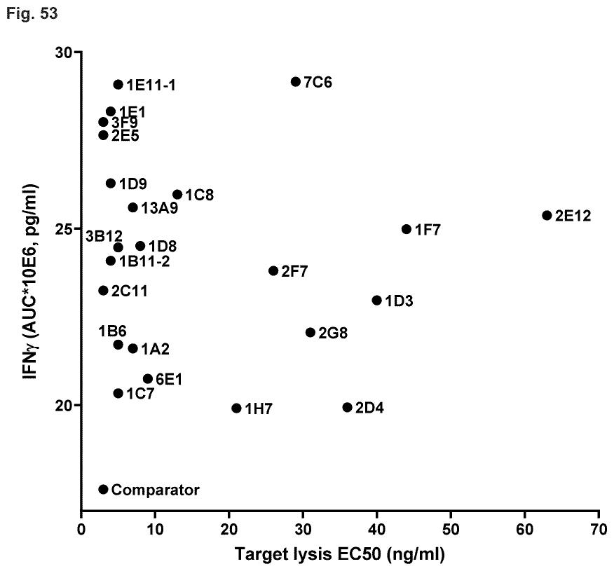

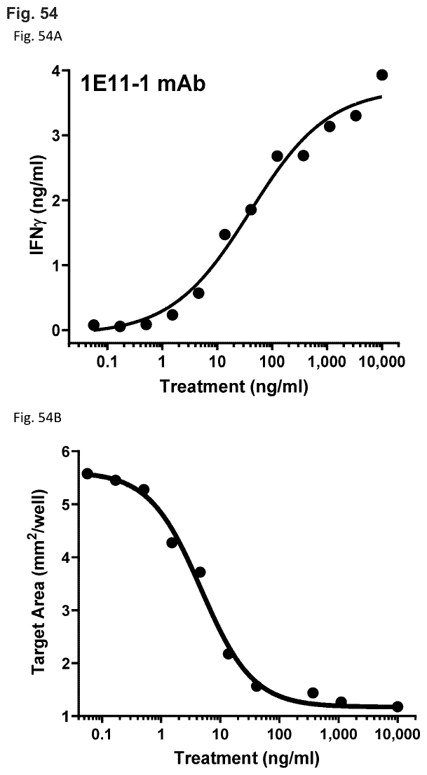

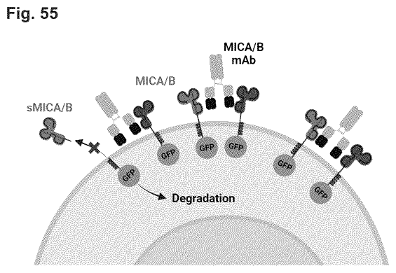

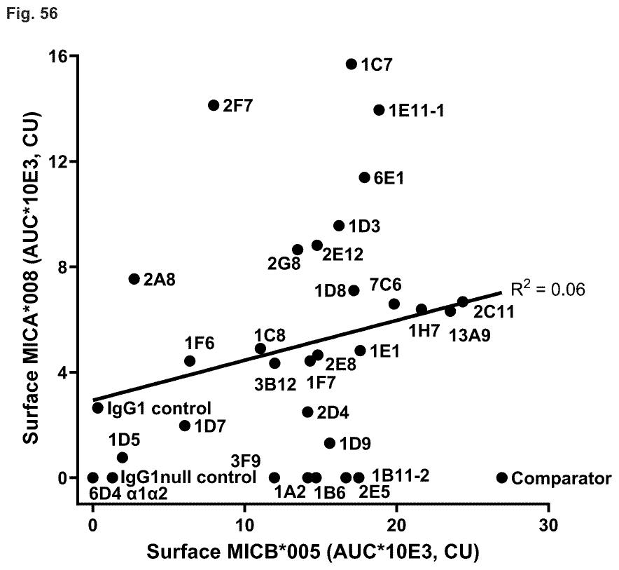

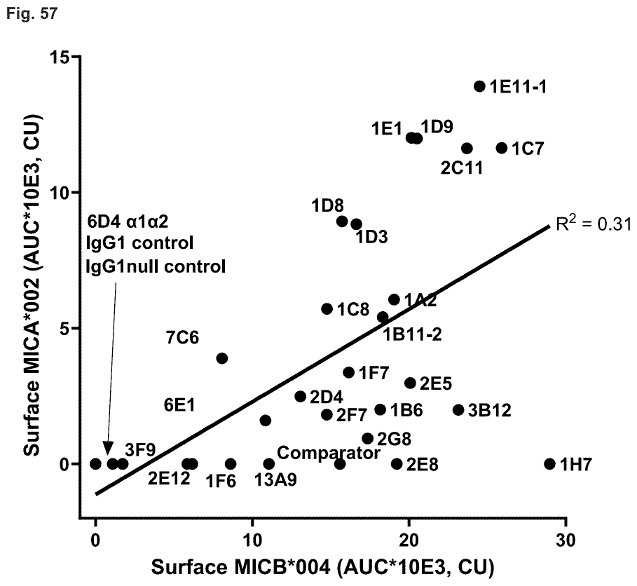

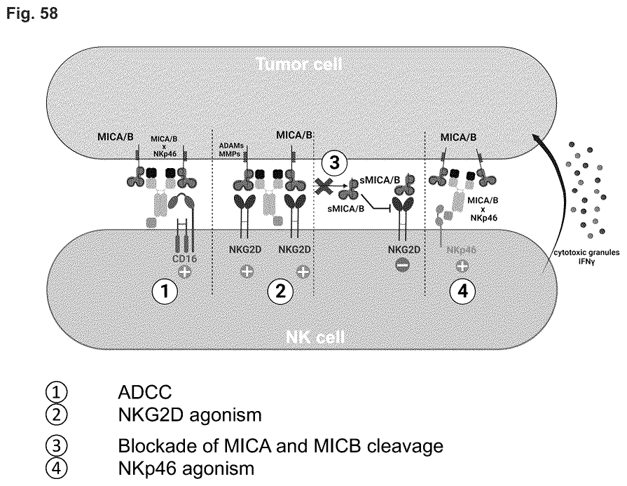

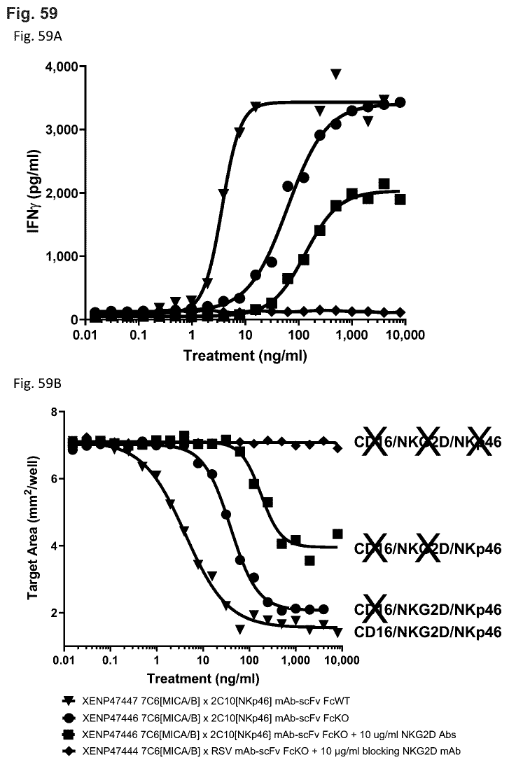

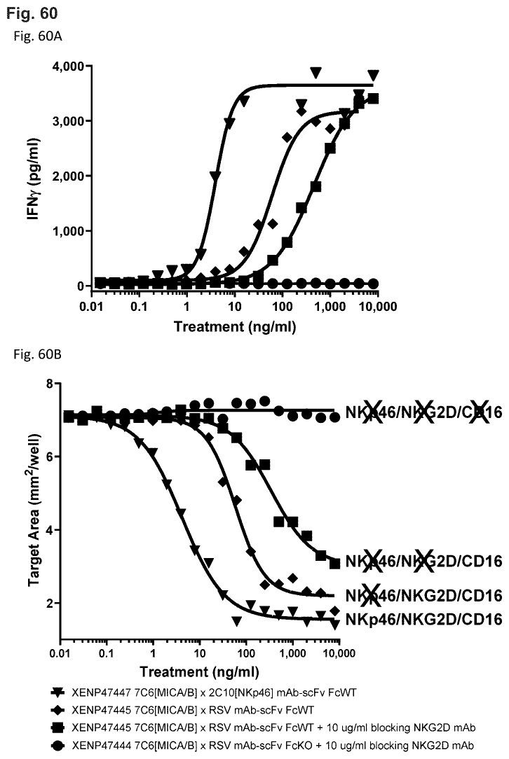

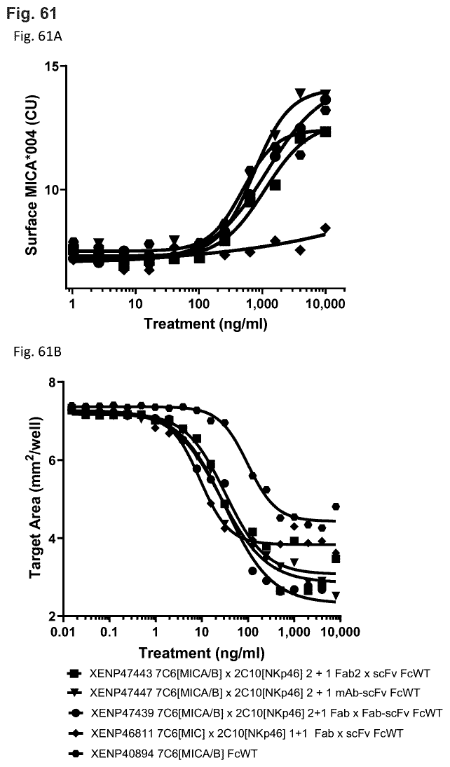

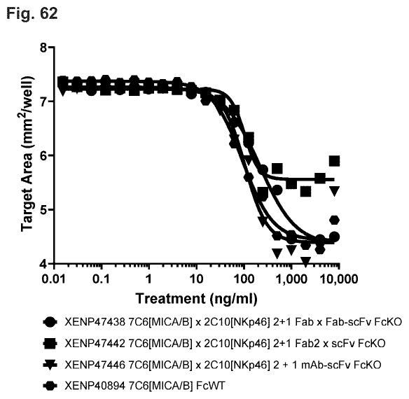

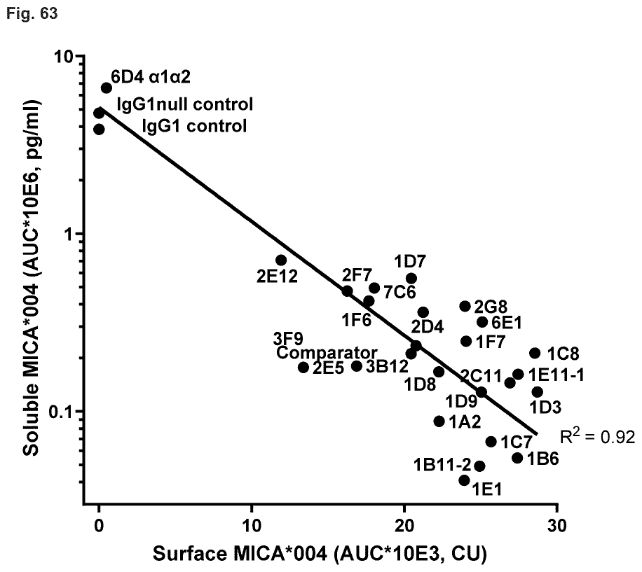

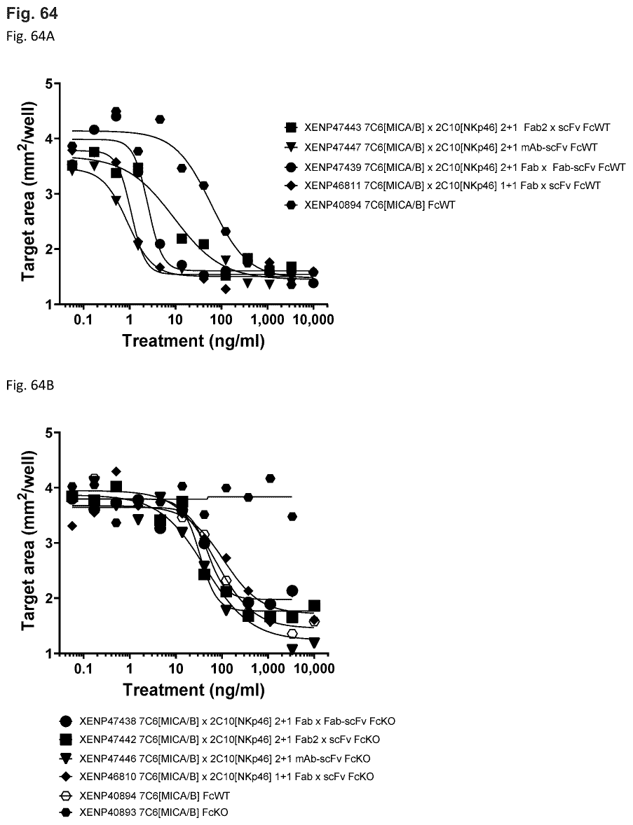

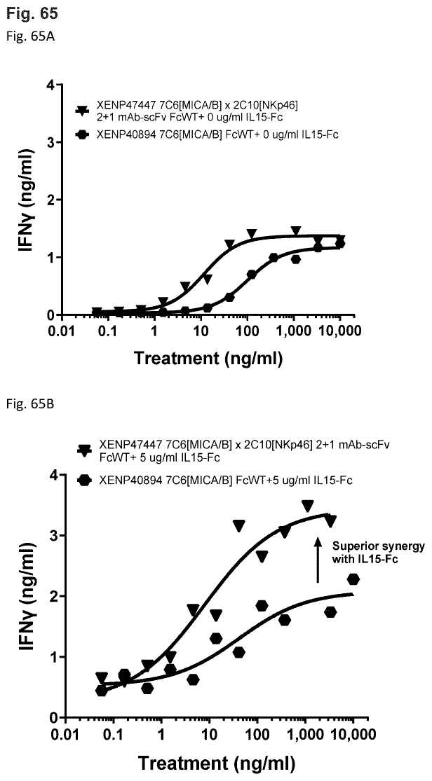

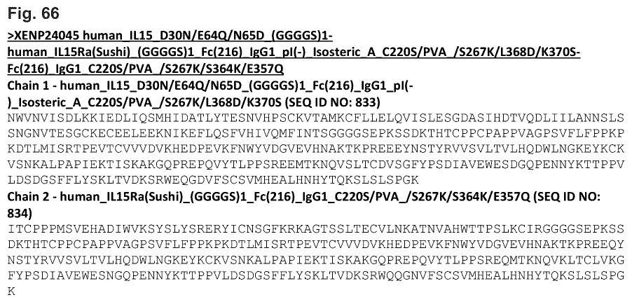

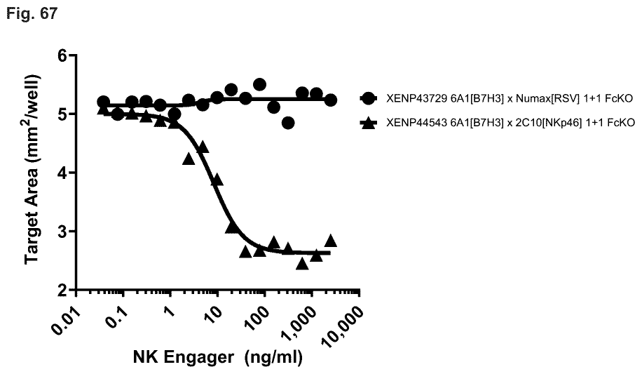

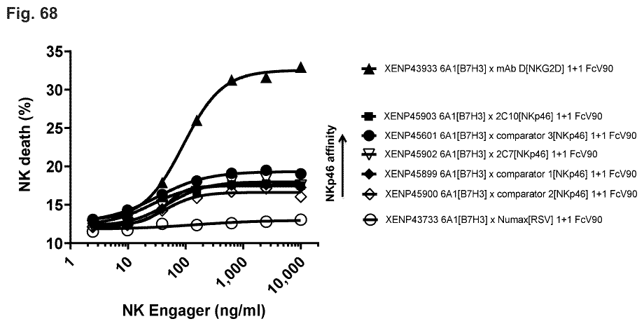

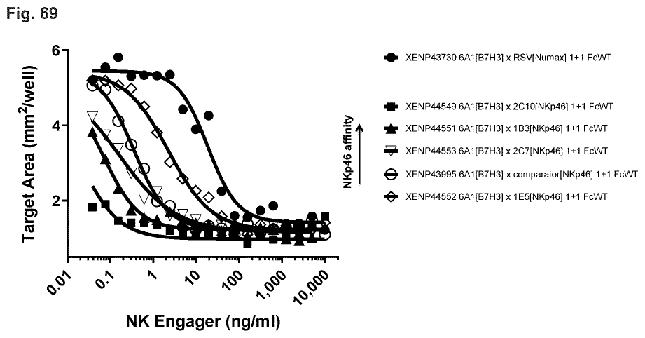

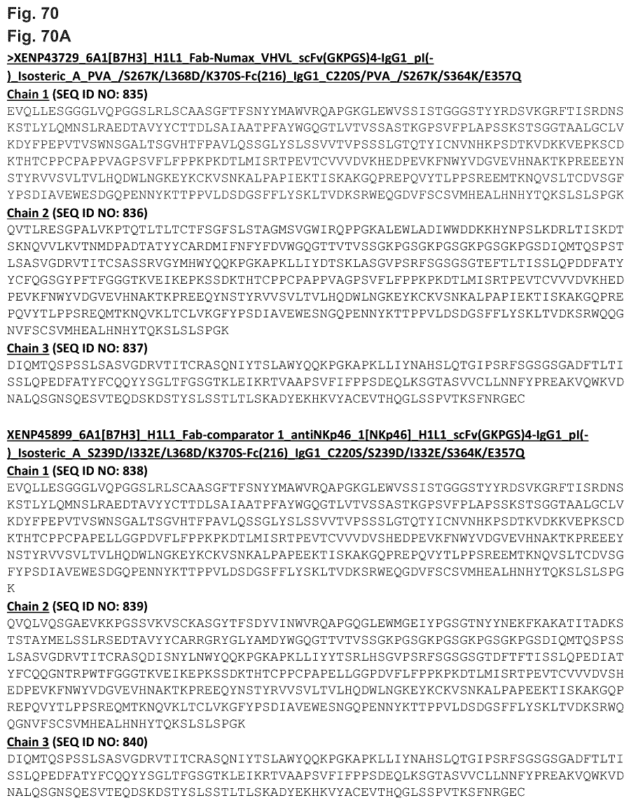

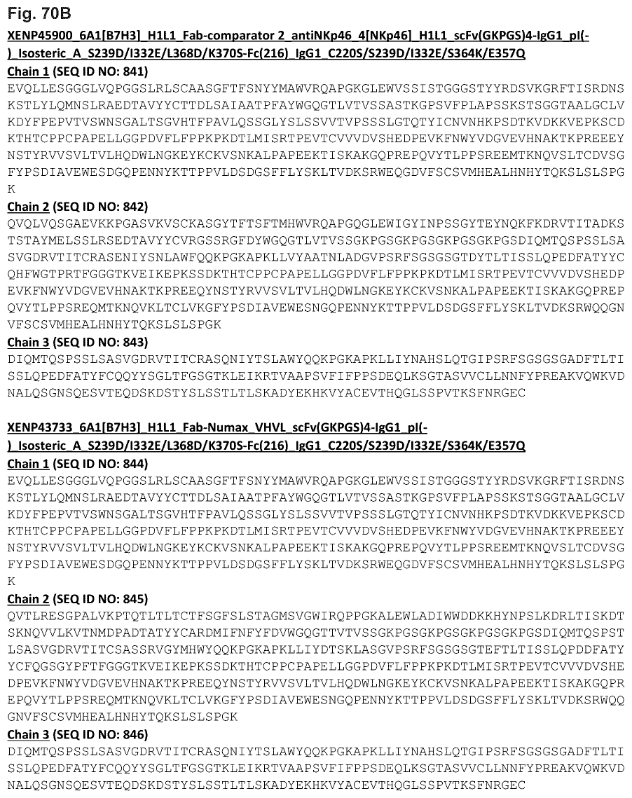

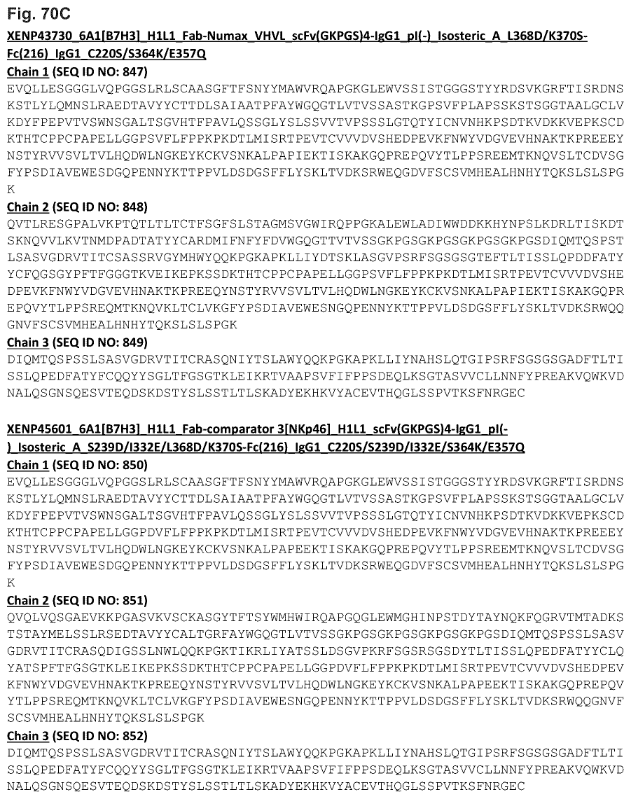

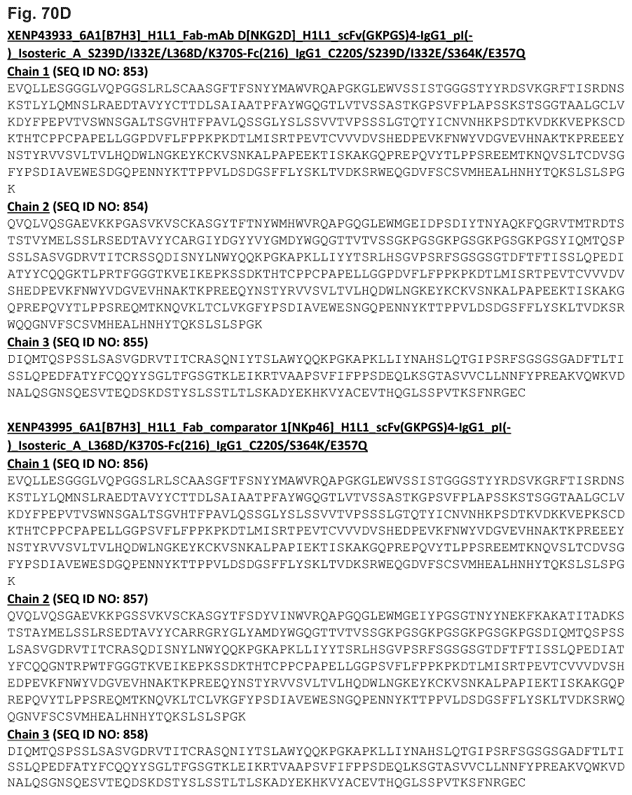

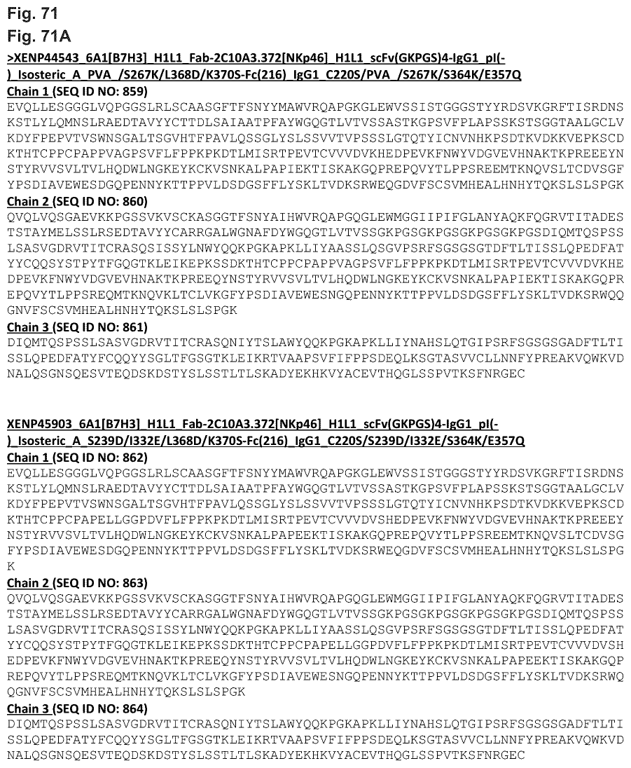

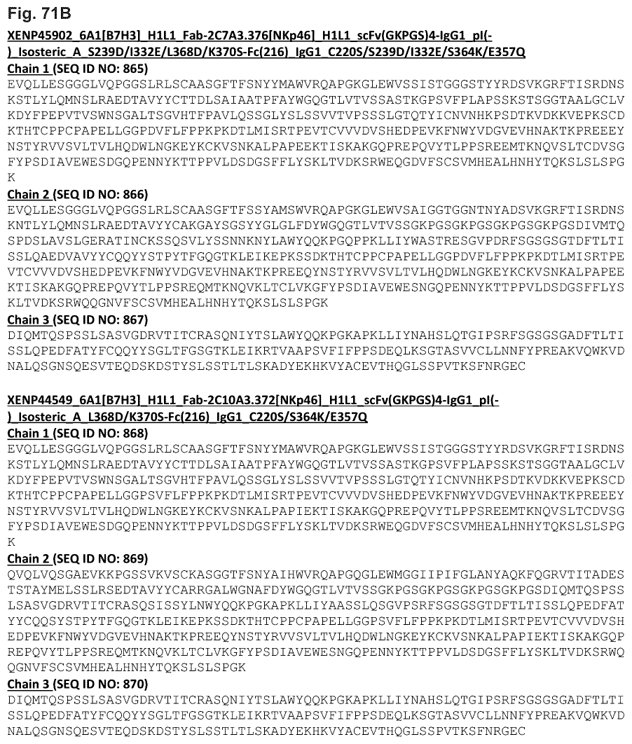

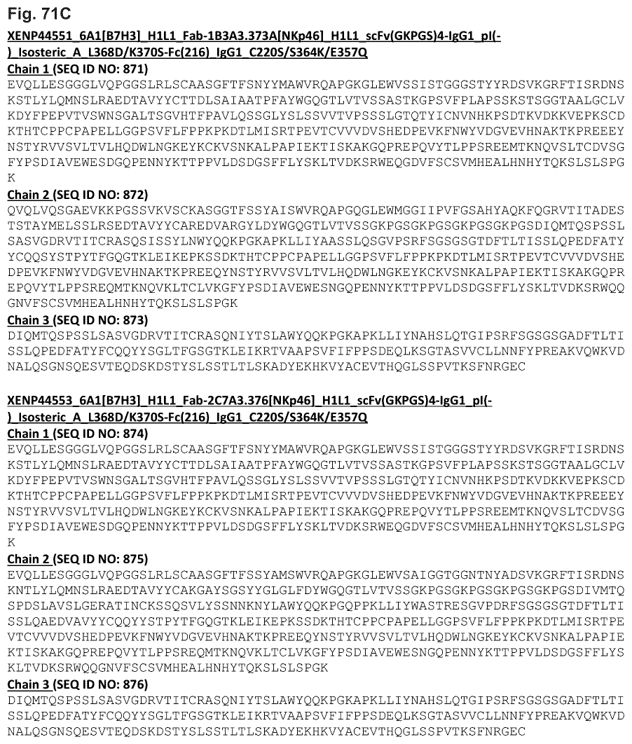

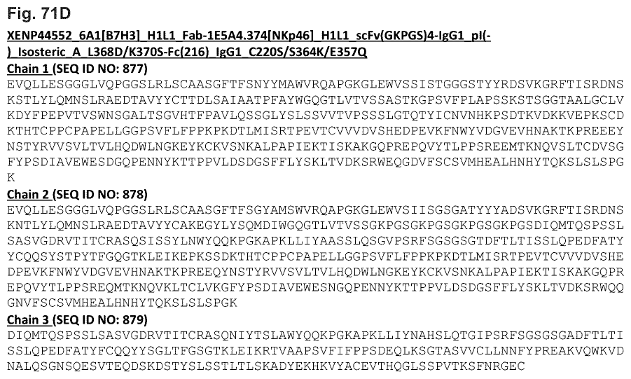

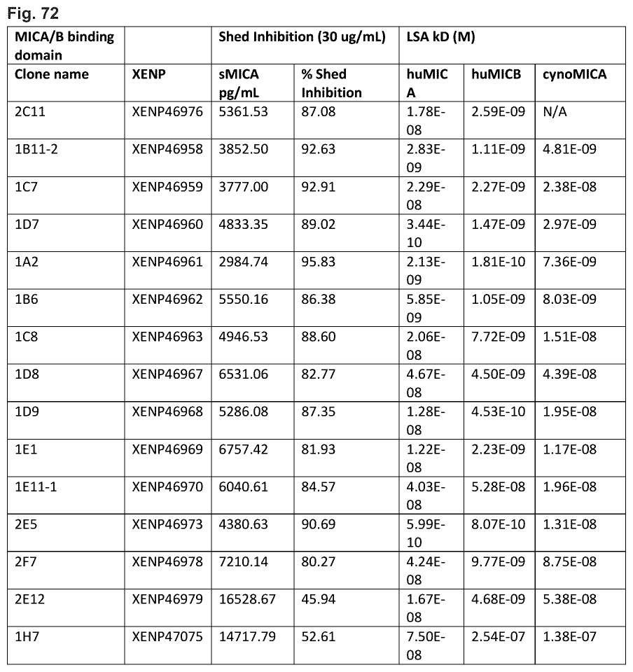

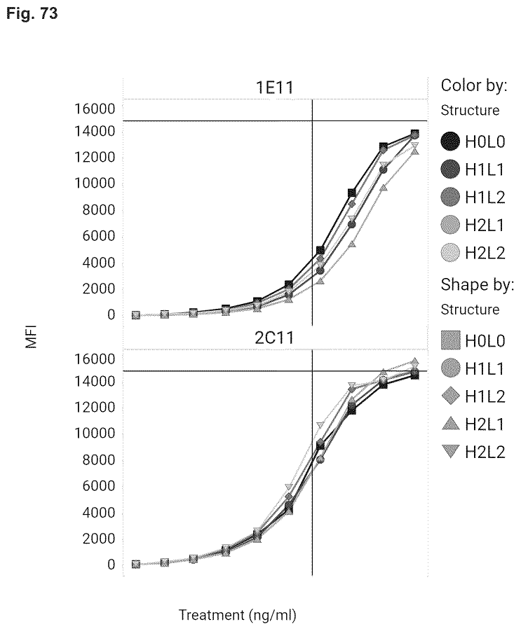

FIG. 16 (FIGS. 16A-16G) depicts illustrative sequences of heterodimeric NKp46×MICA/B bsAb backbone for use in the 2+1 mAb-scFv format. It should be noted that any of the additional backbones depicted in FIGS. 12-15 may be adapted for use in the 2+1 mAb-scFv format with or without including K447_on one or both chains.

FIG. 17 depicts sequences for “CH1” that find use in embodiments of NKp46×MICA/B bsAbs.

FIG. 18 depicts sequences for “hinge” that find use in embodiments of NKp46×MICA/B bsAbs.

FIG. 19 depicts the constant domain of the cognate light chains which find use in the subject NKp46×MICA/B bsAbs that utilize a Fab binding domain.

FIG. 20 depicts soluble MICA/B (sMICA/B) binding NKG2D receptor on NK cells inhibiting signaling. MICA/B bsAbs of the invention may engage cell surface MICA/B and prevent shedding as soluble MICA/B.

FIG. 21 (FIGS. 21A-21S) depicts the variable heavy and variable light chain sequences for novel MICA/B binding domains which may find use in the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 22 (FIGS. 22A and 22B) depicts the variable heavy and variable light chain sequences for additional MICA/B binding domains which may find use in the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 23 depicts the variable heavy and variable light chain sequences for 2C10A3.372, a novel phage-derived NKp46 binding domain. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 24 depicts the variable heavy and variable light chain sequences for additional NKp46 binding domains which may find use in the invention. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 25 (FIGS. 25A-25E) depicts a few of the formats of the present invention. FIG. 25A depicts the “1+1 Fab×scFv” format, with a first Fab arm binding a first antigen and a second scFv arm binding second antigen. The 1+1 Fab-scFv-Fc format comprises a first monomer comprising a first heavy chain variable region (VH1) covalently attached to the N-terminus of a first heterodimeric Fc backbone (optionally via a linker), a second monomer comprising a single-chain Fv covalently attached to the N-terminus of a second corresponding heterodimeric Fc backbone (optionally via a linker), and a third monomer comprising a light chain variable region attached covalently to a light chain constant domain, wherein the light chain variable region is complementary to the VH1. FIG. 25B depicts the “1+1 empty×Fab-scFv” format, with a first Fab arm binding a first antigen and a second scFv arm binding a second antigen. The 1+1 empty×Fab-scFv format comprises a first monomer comprising a first heterodimeric Fc backbone, a second monomer comprising a heavy chain variable region (VH1) covalently attached (optionally via a linker) to a single-chain Fv covalently attached (optionally via a linker) to the N-terminus of a second corresponding heterodimeric Fc backbone, and a third monomer comprising a light chain variable region attached covalently to a light chain constant domain, wherein the light chain variable region is complementary to the VH1. FIG. 25C depicts the “2+1 Fab×Fab-scFv” format, with a first Fab arm and a second Fab-scFv arm, wherein the Fabs bind a first antigen and the scFv binds second antigen. The 2+1 Fab×Fab-scFv format comprises a first monomer comprising a first heavy chain variable region (VH1) covalently attached to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region attached covalently to a light chain constant domain, wherein the light chain variable region is complementary to the VH1. FIG. 25D depicts the “2+1 Fab2×scFv” format, with a first Fab-Fab arm and a second scFv arm, wherein the Fabs bind a first antigen and the scFv binds second antigen. The 2+1 Fab2×scFv format comprises a first monomer comprising a first heavy chain variable region (VH1) covalently attached to a CH1 domain covalently attached to a second VH1 (optionally via a linker) that is further covalently attached to the N-terminus of a first heterodimeric Fc backbone (optionally via a linker), a second monomer comprising a single-chain Fv covalently attached (optionally via a linker) to the N-terminus of a second corresponding heterodimeric Fc backbone, and a third monomer comprising a light chain variable region attached covalently to a light chain constant domain, wherein the light chain variable region is complementary to the VH1. FIG. 25E depicts the “2+1 mAb-scFv” format, with a first Fc comprising an N-terminal Fab arm binding a first antigen and a second Fc comprising an N-terminal Fab arm binding the first antigen and a C-terminal scFv binding a second antigen. The 2+1 mAb-scFv format comprises a first monomer comprising VH1-CH1-hinge-CH2-CH3, a second monomer comprising VH1-CH1-hinge-CH2-CH3-scFv, and a third monomer comprising VL-CL. The VL pairs with the first and second VH1 to form binding domains with binding specificity for the first antigen. In some bsAbs, the first antigen is NKp46 and the second antigen is MICA/B; while in other bsAbs, the first antigen is MICA/B and the second antigen is NKp46.

FIG. 26 (FIGS. 26A and 26B) depicts the sequences of control molecules.

FIG. 27 depicts a proposed mechanism of action for the MICA/B bsAbs of the invention. 1) the bsAb engages MICA/B and tumor antigen on cancer cells, 2) the bsAb engages and activates CD16 on NK cells, and 3) the MICA/B bound by the bsAb engages and signals via NKG2D on NK cells.

FIG. 28 (FIGS. 28A-28H) depicts sequences for B7H3 antibodies comprising Fc variants to enhance ADCC (or WT effector function in the case of XENP41021).

FIG. 29 depicts a matrix of symmetric and asymmetric ADCC-enhanced Fc variants that have been engineered, as well as the corresponding Tm data, affinity data, production yield, ADCC activity and target cell killing activity. As shown, each Fc monomer (−Fc HC or +Fc-scfv-Fc) has either the S239D and I332E (V90) variants, the S239D variant alone, the I332E variant alone, or is wild-type at the 239 and 332 positions; and each test article has a different combination of these Fc monomers.

FIG. 30 depicts the range of ADCC activity of the various symmetric and asymmetric V90 variants outlined in FIG. 29. The results show a large range in levels of fold change in ADCC activity of each construct compared to wild type, with V90 having one of the highest fold changes in ADCC activity compared to WT, and the various S239D and I332E combinations showing a broad range of intermediate levels fold changes.

FIG. 31 (FIGS. 31A and 31B) depicts the sequences or illustrative NKp46×MICA/B bsAbs in the 1+1 Fab×scFv format. CDRs are underlined and slashes indicate the border(s) between the variable regions, linkers, Fc regions, and constant domains. It should be noted that the NKp46×MICA/B bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum. Further, each sequence outlined herein can include or exclude S239D and/or 1332E variants in one or both Fc domains, which results in enhanced ADCC.

FIG. 32 depicts the sequences or illustrative NKp46×MICA/B bsAbs in the 1+1 empty×Fab-scFv format. CDRs are underlined and slashes indicate the border(s) between the variable regions, linkers, Fc regions, and constant domains. It should be noted that the NKp46×MICA/B bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum. Further, each sequence outlined herein can include or exclude S239D and/or 1332E variants in one or both Fc domains, which results in enhanced ADCC.

FIG. 33 depicts the sequences or illustrative NKp46×MICA/B bsAbs in the 2+1 Fab×Fab-scFv format. CDRs are underlined and slashes indicate the border(s) between the variable regions, linkers, Fc regions, and constant domains. It should be noted that the NKp46×MICA/B bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum. Further, each sequence outlined herein can include or exclude S239D and/or I332E variants in one or both Fc domains, which results in enhanced ADCC.

FIG. 34 depicts the sequences or illustrative NKp46×MICA/B bsAbs in the 2+1 Fab2×scFv format. CDRs are underlined and slashes indicate the border(s) between the variable regions, linkers, Fc regions, and constant domains. It should be noted that the NKp46×MICA/B bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum. Further, each sequence outlined herein can include or exclude S239D and/or 1332E variants in one or both Fc domains, which results in enhanced ADCC.

FIG. 35 depicts the sequences or illustrative NKp46×MICA/B bsAbs in the 2+1 mAb-scFv format. CDRs are underlined and slashes indicate the border(s) between the variable regions, linkers, Fc regions, and constant domains. It should be noted that the NKp46×MICA/B bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum. Further, each sequence outlined herein can include or exclude S239D and/or 1332E variants in one or both Fc domains, which results in enhanced ADCC.

FIG. 36 depicts epitope binning of novel MICA/B binding domains (as well as several prior art MICA/B binding domains).

FIG. 37 shows surface MICA upregulation is inversely correlated with soluble MICA. Therefore, surface MICA was used as a readout for screening novel MICA/B binding domains for their ability to prevent MICA and MICB shedding.

FIG. 38 depicts upregulation of surface MICA*002 versus surface MICA*008 to screen MICA/B binding domains that are able to prevent shedding of multiple MICA allelic variants.

FIG. 39 depicts upregulation of surface MICB*004 versus surface MICB*005 to screen MICA/B binding domains that are able to prevent shedding of multiple MICB allelic variants.

FIG. 40 depicts upregulation of surface MICA*004 under normoxic conditions versus hypoxic conditions.

FIG. 41 depicts lysis of MCF7 target cells (4:1 E:T) by NK cells following 72 hours treatment with the novel MICA/B binding domains.

FIG. 42 depicts production of IFNγ by NK cells in co-culture with MCF7 target cells (10:1 E:T) following 24 hours treatment with the novel MICA/B binding domains.

FIG. 43 depicts blockade of soluble MICA shedding from CHO cells engineered to express MICA*004-GFP by novel MICA/B binding domains.

FIG. 44 depicts binding to MICA*004 over-expressed in A375 cell line by novel MICA/B binding domains.

FIG. 45 depicts surface upregulation of MICA*004 expressed in CHO cell line engineered to express MICA*004-GFP following treatment with novel MICA/B binding domains under normoxic conditions.

FIG. 46 depicts surface upregulation of MICA*004 expressed in CHO cell line engineered to express MICA*004-GFP following treatment with novel MICA/B binding domains under hypoxic conditions.

FIG. 47: Pan tumor TMA was stained with MICA/B antibodies showing that breast (top left panel), testis (top right panel), esophagus (bottom left panel), and stomach (bottom right panel) show tumor MICA/B expression. Pancreas, skin, lung, and ovary are additional tumor histologies showing tumor MICA/B expression (not shown).

FIG. 48 depicts MICA/B mAbs mechanisms of action. 1) ADCC, 2) NKG2D agonism, and 3) blockade of MICA and MICB cleavage.

FIG. 49 depicts tumor cell killing (FIG. 49A) and induction of IFNγ secretion (FIG. 49B) by NK cells co-cultured with A375-B2M-KO-RP tumor cell line and dose titration of MICA/B mAb alone, MICA/B mAb with blocking NKG2D mAb, RSV isotype control mAb alone, or RSV mAb with blocking NKG2D mAb.

FIG. 50 depicts blockade of soluble MICA (FIG. 50A) and blockade of soluble MICB (FIG. 50B) after incubating CHO engineered to express MICA or MICB with MICA/B mAb, RSV mAb, or batimastat.

FIG. 51 depicts population frequencies of MICA (FIG. 51A) and MICB (FIG. 51B) allelic variants.

FIG. 52 depicts α3 domain sequence alignment to identify higher frequency MICA and MICB variants. Figure discloses SEQ ID NOS 827-832, respectively, in order of appearance.

FIG. 53 depicts correlation between IFNγ AUC and target lysis EC50 of different MICA/B mAbs.

FIG. 54 depicts target lysis (FIG. 54A) and induction of IFNγ secretion (FIG. 54B) by NK cells co-cultured with MCF7-RFP cells and 1E11-1-based mAb.

FIG. 55 depicts cartoon illustrating surface MICA (MICB) density, measured via C-terminal GFP intensity, inversely correlates with the membrane MICA (MICB) cleavage.

FIG. 56 depicts upregulation of surface MICA*008 versus surface MICB*005 to screen MICA/B binding domains that are able to prevent shedding of MICA and MICB.

FIG. 57 depicts upregulation of surface MICA*002 versus surface MICB*004 to screen MICA/B binding domains that are able to prevent shedding of MICA and MICB.

FIG. 58 depicts hypothesized NKp46×MICA/B mechanisms of action including 1) ADCC, 2) NKG2D agonism, 3) blockade of MICA and MICB cleavage, and 4) NKp46 agonism.

FIG. 59 depicts induction of IFNγ secretion (FIG. 59A) and target cell lysis (FIG. 59B) by NK cells co-cultured with A375-B2M-KO-RFP cells and NKp46×MICA/B bsAbs with WT or KO effector function with or without NKG2D antibody that blocks MICA binding to the receptor.

FIG. 60 depicts induction of IFNγ secretion (FIG. 60A) and target cell lysis (FIG. 60B) by NK cells co-cultured with A375-B2M-KO-RFP cells and NKp46×MICA/B or RSV×MICA/B bsAbs with WT or KO effector function with or without NKG2D antibody that blocks MICA binding to the receptor.

FIG. 61 depicts surface MICA density (indicative of blockade of MICA cleavage) (FIG. 61A) and target cell lysis (FIG. 61B) after co-culturing NK cells with A375-B2M-KO-RFP cells and NKp46×MICA/B in various formats.

FIG. 62 depicts target cell lysis after co-culturing NK cells with A375-B2M-KO-RFP cells and NKp46×MICA/B in various formats with KO effector function.

FIG. 63: Surface MICA upregulation inversely correlates with soluble MICA. Therefore, surface MICA was used as a readout for screening novel MICA/B binding domains for their ability to prevent MICA and MICB shedding.

FIG. 64 depicts target cell lysis after co-culturing NK cells with A375-B2M-KO-RFP cells and NKp46×MICA/B in various formats with WT (FIG. 64A) or KO (FIG. 64B) effector function.

FIG. 65 depicts induction of IFNγ secretion by NK cells co-cultured with A375-B2M-KO-RFP cells and NKp46×MICA/B bsAb without IL-15-Fc fusion (FIG. 65A) and in combination with IL-15-Fc fusion (FIG. 65B).

FIG. 66 depicts sequences for an illustrative IL-15-Fc fusion that may be combined with the NKp46×MICA/B bsAbs of the invention.

FIG. 67 depicts NKp46 mediation of target cell lysis in the absence of CD16 engagement. Test articles XENP43729 (B7H3×RSV control bsAb) and XENP44543 (B7H3×NKp46 bsAb), both having an IgG1 Fc domain that is null for CD16 binding (FcKO), were mixed with purified NK cells and added to a plate of MCF7 target cells at a 3:1 E:T ratio. MCF7 cell lysis was measured by Incucyte at 3-hour intervals.

FIG. 68 depicts the comparatively lower fratricide levels induced by NKp46×B7H3 bsAbs compared to NKG2D×B7H3 bsAb XENP43933. In this experiment, NK cells were mixed with the indicated dilutions of test articles, incubated, stained with Zombie Aqua for 15 minutes and washed before analysis via flow cytometry.

FIG. 69 depicts the target cell lysis induced by B7H3×NKp46 bsAbs of varying affinities. Test articles were mixed with purified NK cells and added to a plate of MCF7 target cells at a 3:1 E:T ratio. MCF7 cell lysis was measured by Incucyte at 3-hour intervals. As depicted, stronger NKp46 affinity correlates with greater levels of target cell lysis.

FIG. 70 (FIGS. 70A-70D) depicts the sequences for various comparator and control antibodies.

FIG. 71 (FIGS. 71A-71D) depicts the sequences for several B7H3×NKp46 antibodies of the invention.

FIG. 72 depicts binding data of MICA/B binding domains of the invention, both from an ELISA measuring the amount of soluble/shed MICA/B, as well as KD data from the Carterra® LSA.

FIG. 73 depicts the comparable binding of murine(H0L0) and humanized (H1L1, H1L2, H2L1, and H2L2) variants of 1E11 and 2C11 to CHO cells expressing MICA*004.

### DETAILED DESCRIPTION

### I. Overview

The description is presented to enable one of ordinary skill in the art to make and use the invention and is provided in the context of a patent application and its requirements. The section headings used herein are for organization purposes only and are not to be construed as limiting the subject matter described. While various embodiments of the invention(s) of the present disclosure have been shown and described herein, it will be obvious to those skilled in the art that such embodiments are provided by way of example only. Numerous variations, changes, and substitutions may occur to those skilled in the art without departing from the invention(s). It should be understood that various alternatives to the embodiments of the invention(s) described herein may be employed in practicing any one of the inventions(s) set forth herein.

All patents, published patent applications, other publications, and sequences from GenBank, and other databases referred to herein are incorporated by reference in their entirety with respect to the related technology.

### II. Nomenclature

The antibodies provided herein are listed in several different formats. In some instances, each monomer of a particular antibody is given a unique “XENP” number, although as will be appreciated in the art, a longer sequence might contain a shorter one. For example, a “scFv-Fc” monomer of a 1+1 Fab-scFv-Fc format antibody may have a first XENP number, while the scFv domain itself will have a different XENP number. Some molecules have three polypeptides, so the XENP number, with the components, is used as a name. Thus, the molecule XENP46810, which is in 1+1 Fab-scFv-Fc format, comprises three sequences (see FIG. 31A) a “Fab-Fc Heavy Chain” monomer (“Chain 1”); 2) a “scFv-Fc Heavy Chain” monomer (“Chain 2”); and 3) a “Light Chain” monomer (“Chain 3”) or equivalents, although one of skill in the art would be able to identify these easily through sequence alignment. These XENP numbers are in the sequence listing as well as identifiers, and used in the Figures. In addition, one molecule, comprising the three components, gives rise to multiple sequence identifiers. For example, the listing of the Fab includes, the full heavy chain sequence, the variable heavy domain sequence and the three CDRs of the variable heavy domain sequence, the full light chain sequence, a variable light domain sequence and the three CDRs of the variable light domain sequence. A Fab-scFv-Fc monomer includes a full-length sequence, a variable heavy domain sequence, 3 heavy CDR sequences, and an scFv sequence (include scFv variable heavy domain sequence, scFv variable light domain sequence and scFv linker). Note that some molecules herein with a scFv domain use a single charged scFv linker (+H), although others can be used. In addition, the naming nomenclature of particular antigen binding domains (e.g., NKp46 and MICA/B binding domains) use a “Hx.xx_Ly.yy” type of format, with the numbers being unique identifiers to particular variable chain sequences. Thus, an Fv domain of the antigen binding domain is “H1 L1,” which indicates that the variable heavy domain, H1, was combined with the light domain L1. In the case that these sequences are used as scFvs, the designation “H1 L1,” indicates that the variable heavy domain, H1 is combined with the light domain, L1, and is in V_{H}-linker-V_{L} orientation, from N- to C-terminus. This molecule with the identical sequences of the heavy and light variable domains but in the reverse order (V_{L}-linker-V_{H} orientation, from N- to C-terminus) would be designated “L1_H1.1”. Similarly, different constructs may “mix and match” the heavy and light chains as will be evident from the sequence listing and the figures.

### III. Definitions

In order that the application may be more completely understood, several definitions are set forth below. Such definitions are meant to encompass grammatical equivalents.

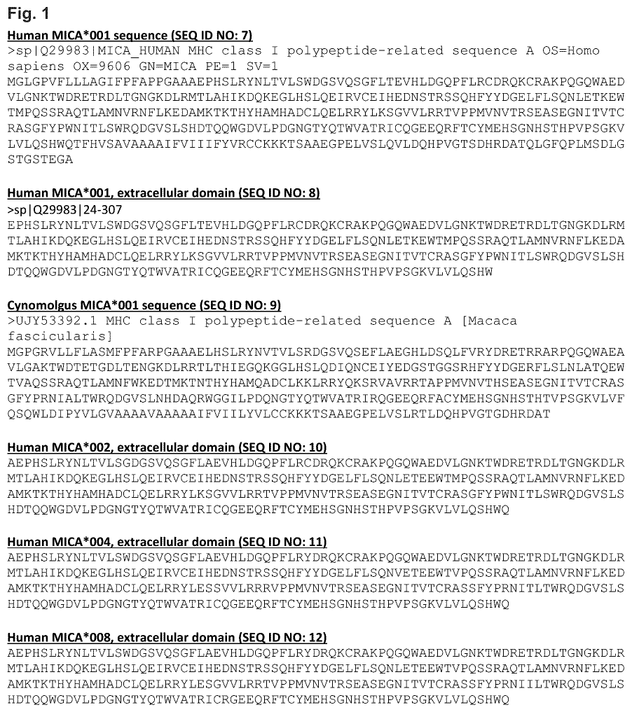

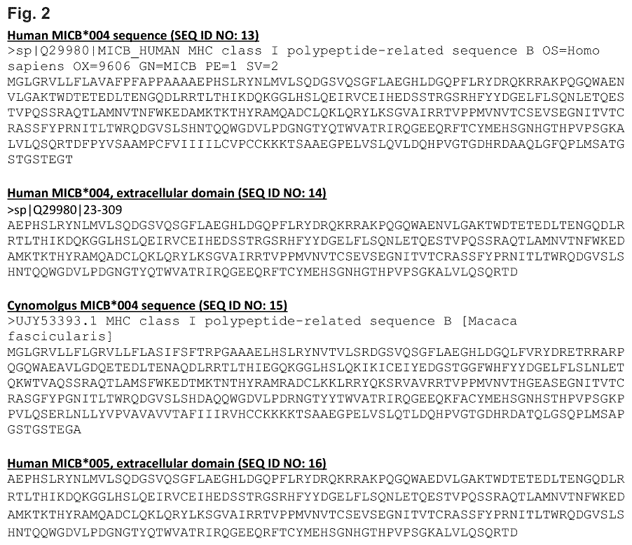

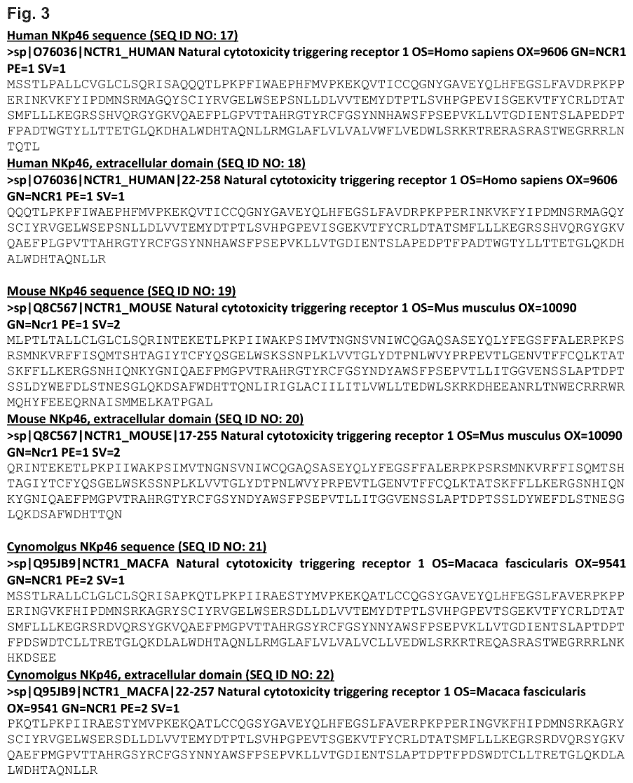

“MICA and MICB” and “MICA/B” is herein meant NKG2D ligands which can, in some instances, be upregulated in several human cancers. MICA/B are transmembrane proteins with MHC-like extracellular domains that, do not associate with beta-2 microglobulin nor present antigens. The proteins can undergo proteolytic cleavage in a multistep process and thereafter, the soluble MICA/B can bind NKG2D on NK cells. In some cases, the soluble MICA/B is shed in the blood plasma and serum in subjects with cancer. Additional information includes amino acid sequences of “MHC class I polypeptide-related sequence A,” “MIC-A” or “MICA” can be found in, for example, UniProt No. Q29983 (human), GenBank Accession Numbers NP_000238.1 (human), NP_001170990.1 (human), NP_001276081 (human), NP_001276082 (human), and NP_001276082 (human). Additional information includes amino acid sequences of “MHC class I polypeptide-related sequence B,” “MIC-B” or “MICB” can be found in, for example, UniProt No. Q29980 (human), GenBank Accession Numbers NP_001276089 (human), NP_001276090 (human), NP_005922 (human), NP_715641 (mouse), and NP_715642 (mouse). Exemplary MICA/B sequences are depicted in FIGS. 1 and 2. Unless otherwise noted, references to MICA/B are to the human MICA/B sequences.

By “NKp46,” “NCR1,” “CD335,” “LY94,” “NK-p46,” “natural cytotoxicity triggering receptor 1” (e.g., GenBank Accession Numbers NM_004829.7 (human), NM_001145457.3 (human), NM_001145458.3 (human), NM_001242356.3 (human), NM_001242357.3 (human), NM_010746.3 (mouse), and NM_001368364.1 (mouse)), is meant a transmembrane protein belonging to the natural cytotoxicity receptor family. Exemplary NKp46 sequences are depicted in FIG. 3. Unless otherwise noted, references to NKp46 are to the human NKp46 sequences.

By “NKG2D,” “NKG2-D,” “natural killer group 2D,” “CD314,” (e.g., GenBank Accession Numbers NP_031386.2 (human), NP_001186734.1 (human), and NP_001076791.1 (mouse)), herein is meant a transmembrane protein belonging to the NKG2 family of C-type lectin-like receptors. NKG2D is a major recognition receptor for the detection and elimination of transformed and/or infected cells as its ligands are induced during cellular stress, either as a result of infection or genomic stress, such as in cancer. In humans, NKG2D is expressed by NK cells, γδ T cells, and CD8⁺ αβ T cells.

By “ablation” herein is meant a decrease or removal of activity. Thus, for example, “ablating FcγR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore, SPR or BLI assay.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

As used herein, the term “antibody” is used generally. Antibodies provided herein can take on a number of formats as described herein, including traditional antibodies as well as antibody derivatives, fragments and mimetics, described herein.

Traditional immunoglobulin (Ig) antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

Other useful antibody formats include, but are not limited to, the “1+1 Fab×scFv” (also referred to herein as the “1+1 Fab-scFv-Fc” or “bottle-opener” format), “1+1 empty×Fab-scFv” (also referred to herein as the “one-armed central-scFv” format), “2+1 Fab×Fab-scFv” (also referred to herein as the “2+1 Fab2-scFv-Fc” format), “2+1 Fab2×scFv” (also referred to herein as the “2+1 stab Fab2-scFv-Fc” format), and “2+1 mAb-scFv” (also referred to herein as the “mAb-scFv” format) formats provided herein (see, e.g., FIGS. 25A-25E). Additional useful antibody formats include, but are not limited to: “mAb-Fv,” “central-Fv,” “1+1 common light chain” (CLC), “2+1 CLC,” “one-armed scFv-mAb,” “scFv-mAb,” “dual scFv,” “bispecific mAb,” and “trident” format antibodies as depicted in FIG. 36 of U.S. Publ. App. No. 2022/0289839, hereby incorporated by reference in its entirety and specifically for its disclosure of antibody formats.

Antibody heavy chains typically include a variable heavy (V_{H}) domain, which includes vhCDR1-3, and an Fc domain, which includes a CH2-CH3 monomer. In some embodiments, antibody heavy chains include a hinge and CH1 domain. Traditional antibody heavy chains are monomers that are organized, from N- to C-terminus: V_{H}—CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype. It should be understood that therapeutic antibodies can also comprise hybrids of isotypes and/or subclasses. For example, as shown in US Publication 2009/0163699, incorporated by reference, the present antibodies, in some embodiments, include human IgG1/G2 hybrids.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (C72 and C73), and optionally all or a portion of the hinge region between CH1 (C71) and CH2 (C72). Thus, in some cases, the Fc domain includes, from N- to C-terminal, CH2-CH3 and hinge-CH2-CH3. In some embodiments, the Fc domain is that from IgG1, IgG2, or IgG4, with IgG1 hinge-CH2-CH3 and IgG4 hinge-CH2-CH3 finding particular use in many embodiments. Additionally, in the case of human IgG1 Fc domains, the hinge may include a C220S amino acid substitution. Furthermore, in the case of human IgG4 Fc domains, the hinge may include a S228P amino acid substitution. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus, for IgG the antibody hinge is herein defined to include positions 216 (E216 in IgG1) to 230 (P230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. Many of the antibodies herein have at least one the cysteines at position 220 according to EU numbering (hinge region) replaced by a serine. Generally, this modification is on the “scFv monomer” side (when 1+1 or 2+1 formats are used) for most of the sequences depicted herein, although it can also be on the “Fab monomer” side, or both, to reduce disulfide formation. Specifically included within the sequences herein are one or both of these cysteines replaced (C220S).

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (V_{L}), which includes light chain CDRs vlCDR1-3, and a constant light chain region (often referred to as C_{L} or C_{K}). The antibody light chain is typically organized from N- to C-terminus: V_{L}-C_{L}.

By “antigen binding domain” or “ABD” herein is meant a set of six Complementary Determining Regions (CDRs) that, when present as part of a polypeptide sequence, specifically binds a target antigen (e.g., NKp46 or MICA/B) as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 vhCDR3 variable light CDRs. The CDRs are present in the variable heavy domain (vhCDR1-3) and variable light domain (vlCDR1-3). The variable heavy domain and variable light domain form an Fv region.

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2, and vhCDR3 (sometimes referred to collectively as vhCDR1-3)) and the disclosure of each variable light region is a disclosure of the vlCDRs (e.g., vlCDR1, vlCDR2, and vlCDR3 (sometimes referred to collectively as vlCDR1-3)). A useful comparison of CDR numbering is as below, see Lafranc et al., Dev. Comp. Immunol. 27(1): 55-77 (2003).

TABLE 2

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 23-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The CDRs contribute to the formation of the antigen-binding, or more specifically, epitope binding site of the antigen binding domains and antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and non-conformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

In some embodiments, the six CDRs of the antigen binding domain are contributed by a variable heavy and a variable light domain. In a “Fab” format, the set of 6 CDRs are contributed by two different polypeptide sequences, the variable heavy domain (vh, VH, or V_{H}; containing the vhCDR1, vhCDR2 and vhCDR3) and the variable light domain (vl, VL, or V_{L}; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of all or part of the hinge in the second monomer.

By “variable region” or “variable domain” as used herein is meant the region of an immunoglobulin that comprises one or more Ig domains substantially encoded by any of the V_{κ}, V_{λ}, and/or V_{H} genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the V_{H}, CH1, V_{L}, and C_{L} immunoglobulin domains, generally on two different polypeptide chains (e.g., V_{H}-CH1 on one chain and V_{L}—C_{L} on the other). Fab may refer to this region in isolation, or this region in the context of a bispecific antibody of the invention. In the context of a Fab, the Fab comprises an Fv region in addition to the CH1 and C_{L} domains.

By “Fv” or “Fv fragment” or “Fv region” as used herein is meant the antibody region that comprises the V_{L} and V_{H} domains. Fv regions can be formatted as both Fabs (as discussed above, generally two different polypeptides that also include the constant regions as outlined above) and single chain Fvs (scFvs), where the vl and vh domains are included in a single peptide, attached generally with a linker as discussed herein.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g., H.X L.Y means N- to C-terminal is vh-linker-vl, and L.Y H.X is vl-linker-vh.

Some embodiments of the subject antibodies provided herein comprise at least one scFv domain, which, while not naturally occurring, generally includes a variable heavy domain and a variable light domain, linked together by a scFv linker. As outlined herein, while the scFv domain is generally from N- to C-terminus oriented as V_{H}-scFv linker-V_{L}, this can be reversed for any of the scFv domains (or those constructed using vh and vl sequences from Fabs), to V_{L}-scFv linker-V_{H}, with optional linkers at one or both ends depending on the format.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233 #, E233( ), E233_or E233del designates a deletion of glutamic acid at position 233. Additionally, EDA233- or EDA233 #designates a deletion of the sequence GluAspAla that begins at position 233.

By “variant protein” or “protein variant,” or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST.

“Variant” as used herein also refers to particular amino acid modifications that confer particular function (e.g., a “heterodimerization variant,” “pI variant,” “ablation variant,” etc.).

As described below, in some embodiments the parent polypeptide, for example an Fc parent polypeptide, is a human wild-type sequence, such as the heavy constant domain or Fc region from IgG1, IgG2, or IgG4, although human sequences with variants can also serve as “parent polypeptides,” for example the IgG1/2 hybrid of US Publication 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2 or IgG4.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

In general, variant Fe domains have at least about 80, 85, 90, 95, 96, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides, and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190: 123-136, entirely incorporated by reference). Fc ligands may include undiscovered molecules that bind Fc.

Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor,” “FcγR,” “FcγR,” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide.

Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “target antigen” as used herein is meant the molecule that is bound specifically by the antigen binding domain comprising the variable regions of a given antibody.

By “strandedness” in the context of the monomers of the heterodimeric antibodies of the invention herein is meant that, similar to the two strands of DNA that “match,” heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pl higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a bispecific antibody according to the invention herein is meant a cell that contains the exogeneous nucleic acids encoding the components of the bispecific antibody and is capable of expressing the bispecific antibody under suitable conditions. Suitable host cells are discussed below.

By “wild-type” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters.

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a K_{D} for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where K_{D} refers to a dissociation rate of a particular antibody-antigen interaction. Typically, an antibody that specifically binds an antigen will have a K_{D} that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a K_{A} or Kₐ for an antigen or epitope of at least 20-, 50-, 100-, 500-, 1000-, 5,000-, 10,000- or more times greater for the epitope relative to a control, where K_{A} or Kₐ refers to an association rate of a particular antibody-antigen interaction. Binding affinity is generally measured using a Biacore, SPR or BLI assay.

As used herein, the singular forms “a,” “an,” and “the” include plural referents unless the context clearly dictates otherwise. Thus, for example, reference to “an antigen” includes mixtures of antigens; reference to “a pharmaceutically acceptable carrier” includes mixtures of two or more such carriers, and the like. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A (alone),” and “B (alone)”.

As used herein, the term “about” a value (or parameter) refers to, for example, ±1%, ±2%, ±3%, ±4%, ±5%, 6%, ±7%, ±8%, ±9%, ±10% and the like of a stated value. When referring to a range of values (or parameters), the term “about” refers to +10% of the upper limit and −10% of the lower limit of a stated range of values. When a range of values is provided, it is to be understood that each intervening value between the upper and lower limit of that range, and any other stated or intervening value in that stated range, is encompassed within the scope of the present disclosure. Where the stated range includes upper and/or lower limits, ranges excluding either of those included limits are also included in the present disclosure.

### IV. Antigen Binding Domains

In another aspect, provided herein are anti-NKp46×anti-MICA/B (also referred to herein as “αNKp46×αMICA/B,” “anti-MICA/B×anti-NKp46,” and “αMICA/B×αNKp46”) bispecific antibodies. Such antibodies include at least one NKp46 binding domain and at least one MICA/B binding domain.

Note that unless specified herein, the order of the antigen list in the name does not confer structure. That is to say, for example, an anti-NKp46×anti-MICA/B 1+1 Fab-scFv-Fc antibody can have the scFv bind to NKp46 or MICA/B, although, in some cases, the order specifies structure as indicated.

As is more fully outlined herein, these combinations of antigen binding domains (ABDs) can be in a variety of formats, as outlined below, generally in combinations where one ABD is in a Fab format and the other is in an scFv format. Exemplary formats that are used in the bispecific antibodies provided herein include the 1+1 Fab×scFv, 1+1 empty×Fab-scFv, 2+1 Fab×Fab-scFv, 2+1 Fab2×scFv, and 2+1 mAb-scFv formats (see, e.g., FIGS. 25A-25E). Other useful antibody formats include, but are not limited to, “mAb-Fv,” “central-Fv,” “1+1 common light chain” (CLC), “2+1 CLC,” “one-armed scFv-mAb,” “scFv-mAb,” “dual scFv,” “bispecific mAb,” and “trident” format antibodies as depicted in FIG. 36 of U.S. Publ. App. No. 2022/0289839, hereby incorporated by reference in its entirety and specifically for its disclosure of antibody formats.

In addition, generally, one of the ABDs comprises a scFv as outlined herein, in an orientation from N- to C-terminus of V_{H}-scFv linker-V_{L} or V_{L}-scFv linker-V_{H}. One or both of the other ABDs, according to the format, generally is a Fab, comprising a V_{H} domain on one protein chain (generally as a component of a heavy chain) and a V_{L} on another protein chain (generally as a component of a light chain).

As will be appreciated by those in the art, any set of 6 CDRs or V_{H} and V_{L} domains can be in the scFv format or in the Fab format, which is then added to the heavy and light constant domains, where the heavy constant domains comprise variants (including within the CH1 domain, as well as the Fc domain). The scFv sequences contained in the sequence listing utilize a particular charged linker, but as outlined herein, uncharged or other charged linkers can be used, including those depicted in FIG. 8 (see, e.g., SEQ ID NOs: 23-48).

In addition, as discussed above, the numbering used in the sequence listing for the identification of the CDRs is Kabat; however, different numbering can be used, which will change the amino acid sequences of the CDRs as shown in Table 2.

For all of the variable heavy and light domains listed herein, further variants can be made. As outlined herein, in some embodiments, the set of 6 CDRs can have 0, 1, 2, 3, 4, or 5 amino acid modifications (with amino acid substitutions finding particular use), as well as changes in the framework regions of the variable heavy and light domains, as long as the frameworks (excluding the CDRs) retain at least about 80%, about 85%, about 90%, about 95%, or about 99% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at least about 80%, about 85%, about 90%, about 95%, or about 99% identity to a human germline sequence selected from those listed in FIG. 1 of U.S. Pat. No. 7,657,380. Alternatively, the CDRs can have amino acid modifications (e.g., from 1, 2, 3, 4, or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.)), as well as having framework region changes, as long as the framework regions retain at least about 80%, about 85%, about 90%, about 95%, or about 99% identity to a human germline sequence selected from those listed in FIG. 1 of U.S. Pat. No. 7,657,380.

As discussed herein, the subject heterodimeric antibodies include two ABDs, each of which binds to NKp46 or MICA/B. As outlined herein, these heterodimeric antibodies can be bispecific and bivalent (each antigen is bound by a single ABD, for example, in the format depicted in FIG. 25), or bispecific and trivalent (one antigen is bound by a single ABD and the other is bound by two ABDs, for example, in the format depicted in FIG. 25).

### A. MICA/B Antigen Binding Domains

Herein is provided monoclonal and bispecific antibodies (e.g., the anti-NKp46×anti-MICA/B antibodies provided herein), and fusion proteins that contain ABDs that bind to MICA/B. Suitable sets of 6 CDRs (vhCDR1-3 and vlCDR1-3; see, e.g., SEQ ID NOs: 240-242 and 244-246, 248-250 and 252-254, 256-258 and 260-262, 264-266 and 268-270, 272-274 and 276-278, 280-282 and 284-286, 288-290 and 292-294, 296-298 and 300-302, 304-306 and 308-310, 312-313 and 316-318, 320-322 and 324-326, 328-330 and 332-334, 336-338 and 340-342, 344-346 and 348-350, 352-354 and 356-358, 360-362 and 364-366, 368-370 and 372-374, 376-378 and 380-382, 384-386 and 388-390, 392-394 and 396-398, 400-402 and 404-406, 408-410 and 412-414, 416-418 and 420-422, 424-426 and 428-430, 432-434 and 436-438, 440-442 and 444-446, 448-450 and 452-454, 456-458 and 460-462, 464-466 and 468-470, 472-474 and 476-478, 480-482 and 483-486, 488-490 and 492-494, 496-498 and 500-502, 504-506 and 508-510, 512-514 and 516-518, 520-522 and 524-526, 528-530 and 532-534, 536-538 and 540-542, 544-546 and 548-550, 552-554 and 556-558, 560-562 and 564-566, 568-570 and 572-574, 576-578 and 580-582, 584-586 and 588-590, 592-594 and 596-598, 600-602 and 604-606, 608-610 and 612-614, 616-618 and 620-622, 624-626 and 628-630, 632-634 and 636-638, 640-642 and 644-646, 648-650 and 652-654, 656-658 and 660-662, 664-666 and 668-670, 672-674 and 676-678, 648-650 and 652-654, 680-682 and 684-686, 688-690 and 692-694, 696-698 and 700-702, 704-706 and 708-710, and 712-714 and 716-718, respectively) and/or V_{H} and V_{L} domains (see, e.g., SEQ ID NOs: 239 and 243, 247 and 251, 255 and 259, 263 and 267, 271 and 275, 279 and 283, 287 and 291, 295 and 299, 303 and 307, 311 and 315, 319 and 323, 327 and 331, 335 and 339, 343 and 347, 351 and 355, 359 and 363, 367 and 371, 375 and 379, 383 and 387, 391 and 395, 399 and 403, 407 and 411, 415 and 419, 423 and 427, 431 and 435, 439 and 443, 447 and 451, 455 and 459, 463 and 467, 471 and 475, 479 and 483, 487 and 491, 495 and 499, 503 and 507, 511 and 515, 519 and 523, 527 and 531, 535 and 539, 543 and 547, 551 and 555, 559 and 563, 567 and 571, 575 and 579, 583 and 587, 591 and 595, 599 and 603, 607 and 611, 615 and 619, 623 and 627, 631 and 635, 639 and 643, 647 and 651, 655 and 659, 663 and 667, 671 and 675, 647 and 651, 679 and 683, 687 and 691, 695 and 699, 703 and 707, and 711 and 715, respectively) are depicted in FIGS. 21 and 22. In some embodiments, the heterodimeric antibody is a 1+1 Fab×scFv, 1+1 empty×Fab-scFv, 2+1 Fab×Fab-scFv, 2+1 Fab2×scFv, or 2+1 mAb-scFv format antibody (see, e.g., FIGS. 25 and 31-35).

In some embodiments, the MICA/B ABD has a set of vhCDRs selected from the vhCDR1, vhCDR2, and vhCDR3 sequences from a V_{H} selected from the group including: (i) D94837_1E11_1 [MICA/B]_H0 (SEQ ID NOs: 240-242), (ii) D94837_E11_1 [MICA/B]_H1 (SEQ ID NOs: 248-250 and 256-258), (iii) D94837_1E1_1 [MICA/B]_H2 (SEQ ID NOs: 264-266 and 272-274), (iv) 2E5 [MICA/B]_H0 (SEQ ID NOs: 280-282), (v) 2E5 [MICA/B]_H1 (SEQ ID NOs: 288-290 and 296-298), (vi) 2E5 [MICA/B]_H2 (SEQ ID NOs: 304-306 and 312-314), (vii) D94852_2E12 [MICA/B]_H0 (SEQ ID NOs: 320-322), (viii) D94852_2E12 [MICA/B]_H1 (SEQ ID NOs: 328-330 and 336-338), (ix) D94852_2E12 [MICA/B]_H2 (SEQ ID NOs: 344-346 and 352-354), (x) D99136_2F7 [MICA/B]_H0 (SEQ ID NOs: 360-362), (xi) D99136_2F7 [MICA/B]_H1 (SEQ ID NOs: 368-370 and 376-378), (xii) D99136_2F7 [MICA/B]_H2 (SEQ ID NOs: 384-386 and 392-394), (xiii) D103388_1C7 [MICA/B]_H0 (SEQ ID NOs: 400-402), (xiv) D103388_1C7 [MICA/B]_H1 (SEQ ID NOs: 408-410 and 416-418), (xv) D103388_1C7 [MICA/B]_H2 (SEQ ID NOs: 424-426 and 432-434), (xvi) D103388_1D7 [MICA/B]_H0 (SEQ ID NOs: 440-442), (xvii) D103388_1D7 [MICA/B]_H1 (SEQ ID NOs: 448-450 and 456-458), (xviii) D103388_1D7 [MICA/B]_H2 (SEQ ID NOs: 464-466 and 472-474), (xix) D105317_1A2 [MICA/B]_H0 (SEQ ID NOs: 480-482), (xx) D105317_1A2 [MICA/B]_H1 (SEQ ID NOs: 488-490 and 496-498), (xxi) D105317_1A2 [MICA/B]_H2 (SEQ ID NOs: 504-506 and 512-514), (xxii) D99136_2C11 [MICA/B]_H0 (SEQ ID NOs: 520-522), (xxiii) D99136_2C11 [MICA/B]_H1 (SEQ ID NOs: 528-530 and 536-538), (xxiv) D99136_2C11 [MICA/B]_H2 (SEQ ID NOs: 544-546 and 552-554), (xxv) D103388_1B11-2 [MICA/B]_H0 (SEQ ID NOs: 560-562), (xxvi) D105317_1B6 [MICA/B]_H0 (SEQ ID NOs: 568-570), (xxvii) D105317_1C8 [MICA/B]_H0 (SEQ ID NOs: 576-578), (xxviii) D105317_1F6 [MICA/B]_H0 (SEQ ID NOs: 584-586), (xxix) D105317_1F7 [MICA/B]_H0 (SEQ ID NOs: 592-594), (xxx) D94837_1D3 [MICA/B]_H0 (SEQ ID NOs: 600-602), (xxxi) D94837_1D8 [MICA/B]_H0 (SEQ ID NOs: 608-610), (xxxii) D94837_1D9 [MICA/B]_H0 (SEQ ID NOs: 616-618), (xxxiii) D94837_1E1 [MICA/B]_H0 (SEQ ID NOs: 624-626), (xxxiv) D94852_2D4 [MICA/B]_H0 (SEQ ID NOs: 632-634), (xxxv) D94852_2G8 [MICA/B]_H0 (SEQ ID NOs: 640-642), (xxxvi) D88487_2A8 [MICA/B]_H0 (SEQ ID NOs: 648-650), (xxxvii) D94837_3B12 [MICA/B]_H0 (SEQ ID NOs: 656-658), (xxxviii) D99122_1H7 [MICA/B]_H0 (SEQ ID NOs: 664-666), (xxxix) D99136_2E8 [MICA/B]_H0 (SEQ ID NOs: 672-674), (xl) D88487_2A8 [MICA/B]_H0 (SEQ ID NOs: 648-650), (xli) 3F9[MICA/B]_H0 (SEQ ID NOs: 680-682), (xlii) 6E1[MICA/B]_H0 (SEQ ID NOs: 688-690), (xliii) 7C6[MICA/B]_H0 (SEQ ID NOs: 696-698), (xliv) 13A9 [MICA/B]_H0 (SEQ ID NOs: 704-706), and (xlv) 1D5 [MICA/B]_H0 (SEQ ID NOs: 712-714), as shown in FIGS. 21 and 22. In some embodiments, the vhCDR1, vhCDR2, and vhCDR3 sequences of the MICA/B ABD are SEQ ID NOs: 240-242 (such as, for example, in D94837_1E11_1 [MICA/B]_H0). In other embodiments, the vhCDR1, vhCDR2, and vhCDR3 sequences of the MICA/B ABD are SEQ ID NOs: 248-250 and 256-258 (such as, for example, in D94837_1E11_1 [MICA/B]_H1). In other embodiments, the vhCDR1, vhCDR2, and vhCDR3 sequences of the MICA/B ABD are SEQ ID NOs: 264-266 and 272-274 (such as, for example, in D94837_1E11_1 [MICA/B]_H2). In other embodiments, the vhCDR1, vhCDR2, and vhCDR3 sequences of the MICA/B ABD are SEQ ID NOs: 280-282 (such as, for example, in 2E5 [MICA/B]_H0). In other embodiments, the vhCDR1, vhCDR2, and vhCDR3 sequences of the MICA/B ABD are SEQ ID NOs: 288-290 and 296-298 (such as, for example, in 2E5 [MICA/B]_H1). In other embodiments, the vhCDR1, vhCDR2, and vhCDR3 sequences of the MICA/B ABD are SEQ ID NOs: 304-306 and 312-314 (such as, for example, in 2E5 [MICA/B]_H2). In other embodiments, the vhCDR1, vhCDR2, and vhCDR3 sequences of the MICA/B ABD are SEQ ID NOs: 320-322 (such as, for example, in D94852_2E12 [MICA/B]_H0). In other embodiments, the vhCDR1, vhCDR2, and vhCDR3 sequences of the MICA/B ABD are SEQ ID NOs: 328-330 and 336-338 (such as, for example, in D94852_2E12 [MICA/B]_H1). In other embodiments, the vhCDR1, vhCDR2, and vhCDR3 sequences of the MICA/B ABD are SEQ ID NOs: 344-346 and 352-354 (such as, for example, in D94852_2E12 [MICA/B]_H2). In other embodiments, the vhCDR1, vhCDR2, and vhCDR3 sequences of the MICA/B ABD are SEQ ID NOs: 360-362 (such as, for example, in D99136_2F7 [MICA/B]_H0). In other embodiments, the vhCDR1, vhCDR2, and vhCDR3 sequences of the MICA/B ABD are SEQ ID NOs: 368-370 and 376-378 (such as, for example, in D99136_2F7 [MICA/B]_H1). In other embodiments, the vhCDR1, vhCDR2, and vhCDR3 sequences of the MICA/B ABD are SEQ ID NOs: 384-386 and 392-394 (such as, for example, in D99136_2F7 [MICA/B]_H2). In other embodiments, the vhCDR1, vhCDR2, and vhCDR3 sequences of the MICA/B ABD are SEQ ID NOs: 400-402 (such as, for example, in D103388_1C7 [MICA/B]_H0). In other embodiments, the vhCDR1, vhCDR2, and vhCDR3 sequences of the MICA/B ABD are SEQ ID NOs: 408-410 and 416-418 (such as, for example, in D103388_1C7 [MICA/B]_H1). In other embodiments, the vhCDR1, vhCDR2, and vhCDR3 sequences of the MICA/B ABD are SEQ ID NOs: 424-426 and 432-434 (such as, for example, in D103388_1C7 [MICA/B]_H2). In other embodiments, the vhCDR1, vhCDR2, and vhCDR3 sequences of the MICA/B ABD are SEQ ID NOs: 440-442 (such as, for example, in D103388_1D7 [MICA/B]_H0). In other embodiments, the vhCDR1, vhCDR2, and vhCDR3 sequences of the MICA/B ABD are SEQ ID NOs: 448-450 and 456-458 (such as, for example, in D103388_1D7 [MICA/B]_H1). In other embodiments, the vhCDR1, vhCDR2, and vhCDR3 sequences of the MICA/B ABD are SEQ ID NOs: 464-466 and 472-474 (such as, for example, in D103388_1D7 [MICA/B]_H2). In other embodiments, the vhCDR1, vhCDR2, and vhCDR3 sequences of the MICA/B ABD are SEQ ID NOs: 480-482 (such as, for example, in D105317_1A2 [MICA/B]_H0). In other embodiments, the vhCDR1, vhCDR2, and vhCDR3 sequences of the MICA/B ABD are SEQ ID NOs: 488-490 and 496-498 (such as, for example, in D105317_1A2 [MICA/B]_H1). In other embodiments, the vhCDR1, vhCDR2, and vhCDR3 sequences of the MICA/B ABD are SEQ ID NOs: 504-506 and 512-514 (such as, for example, in D105317_1A2 [MICA/B]_H2). In other embodiments, the vhCDR1, vhCDR2, and vhCDR3 sequences of the MICA/B ABD are SEQ ID NOs: 520-522 (such as, for example, in D99136_2C11 [MICA/B]_H0). In other embodiments, the vhCDR1, vhCDR2, and vhCDR3 sequences of the MICA/B ABD are SEQ ID NOs: 528-530 and 536-538 (such as, for example, in D99136_2C11 [MICA/B]_H1). In other embodiments, the vhCDR1, vhCDR2, and vhCDR3 sequences of the MICA/B ABD are SEQ ID NOs: 544-546 and 552-554 (such as, for example, in D99136_2C11 [MICA/B]_H2). In other embodiments, the vhCDR1, vhCDR2, and vhCDR3 sequences of the MICA/B ABD are SEQ ID NOs: 560-562 (such as, for example, in D103388_1B11-2 [MICA/B]_H0). In other embodiments, the vhCDR1, vhCDR2, and vhCDR3 sequences of the MICA/B ABD are SEQ ID NOs: 568-570 (such as, for example, in D105317_1B6 [MICA/B]_H0). In other embodiments, the vhCDR1, vhCDR2, and vhCDR3 sequences of the MICA/B ABD are SEQ ID NOs: 576-578 (such as, for example, in D105317_1C8 [MICA/B]_H0). In other embodiments, the vhCDR1, vhCDR2, and vhCDR3 sequences of the MICA/B ABD are SEQ ID NOs: 584-586 (such as, for example, in D105317_1F6 [MICA/B]_H0). In other embodiments, the vhCDR1, vhCDR2, and vhCDR3 sequences of the MICA/B ABD are SEQ ID NOs: 592-594 (such as, for example, in D105317_1F7 [MICA/B]_H0). In other embodiments, the vhCDR1, vhCDR2, and vhCDR3 sequences of the MICA/B ABD are SEQ ID NOs: 600-602 (such as, for example, in D94837_1D3 [MICA/B]_H0). In other embodiments, the vhCDR1, vhCDR2, and vhCDR3 sequences of the MICA/B ABD are SEQ ID NOs: 608-610 (such as, for example, in D94837_1D8 [MICA/B]_H0). In other embodiments, the vhCDR1, vhCDR2, and vhCDR3 sequences of the MICA/B ABD are SEQ ID NOs: 616-618 (such as, for example, in D94837_1D9 [MICA/B]_H0). In other embodiments, the vhCDR1, vhCDR2, and vhCDR3 sequences of the MICA/B ABD are SEQ ID NOs: 624-626 (such as, for example, in D94837_1E1 [MICA/B]_H0). In other embodiments, the vhCDR1, vhCDR2, and vhCDR3 sequences of the MICA/B ABD are SEQ ID NOs: 632-634 (such as, for example, in D94852_2D4 [MICA/B]_H0). In other embodiments, the vhCDR1, vhCDR2, and vhCDR3 sequences of the MICA/B ABD are SEQ ID NOs: 640-642 (such as, for example, in D94852_2G8 [MICA/B]_H0). In other embodiments, the vhCDR1, vhCDR2, and vhCDR3 sequences of the MICA/B ABD are SEQ ID NOs: 648-650 (such as, for example, in D88487_2A8 [MICA/B]_H0). In other embodiments, the vhCDR1, vhCDR2, and vhCDR3 sequences of the MICA/B ABD are SEQ ID NOs: 656-658 (such as, for example, in D94837_3B12 [MICA/B]_H0). In other embodiments, the vhCDR1, vhCDR2, and vhCDR3 sequences of the MICA/B ABD are SEQ ID NOs: 664-666 (such as, for example, in D99122_1H7 [MICA/B]_H0). In other embodiments, the vhCDR1, vhCDR2, and vhCDR3 sequences of the MICA/B ABD are SEQ ID NOs: 672-674 (such as, for example, in D99136_2E8 [MICA/B]_H0). In other embodiments, the vhCDR1, vhCDR2, and vhCDR3 sequences of the MICA/B ABD are SEQ ID NOs: 648-650 (such as, for example, in D88487_2A8 [MICA/B]_H0). In other embodiments, the vhCDR1, vhCDR2, and vhCDR3 sequences of the MICA/B ABD are SEQ ID NOs: 680-682 (such as, for example, in 3F9[MICA/B]_H0). In other embodiments, the vhCDR1, vhCDR2, and vhCDR3 sequences of the MICA/B ABD are SEQ ID NOs: 688-690 (such as, for example, in 6E1[MICA/B]_H0). In other embodiments, the vhCDR1, vhCDR2, and vhCDR3 sequences of the MICA/B ABD are SEQ ID NOs: 696-698 (such as, for example, in 7C6[MICA/B]_H0). In other embodiments, the vhCDR1, vhCDR2, and vhCDR3 sequences of the MICA/B ABD are SEQ ID NOs: 704-706 (such as, for example, in 13A9 [MICA/B]_H0). In other embodiments, the vhCDR1, vhCDR2, and vhCDR3 sequences of the MICA/B ABD are SEQ ID NOs: 712-714 (such as, for example, in 1D5 [MICA/B]_H0).

In some embodiments, the V_{H} domain of the MICA/B ABD is selected from the group the group including: (i) D94837_1E11_1 [MICA/B]_H0 (SEQ ID NO: 239), (ii) D94837_1E11_1 [MICA/B]_H1 (SEQ ID NOs: 247 and 255), (iii) D94837_1E11_1 [MICA/B]_H2 (SEQ ID NOs: 263 and 271), (iv) 2E5 [MICA/B]_H0 (SEQ ID NO: 279), (v) 2E5 [MICA/B]_H1 (SEQ ID NOs: 287 and 295), (vi) 2E5 [MICA/B]_H2 (SEQ ID NOs: 303 and 311), (vii) D94852_2E12 [MICA/B]_H0 (SEQ ID NO: 319), (viii) D94852_2E12 [MICA/B]H1 (SEQ ID NOs: 327 and 335), (ix) D94852_2E12 [MICA/B]_H2 (SEQ ID NOs: 343 and 351), (x) D99136_2F7 [MICA/B]_H0 (SEQ ID NO: 359), (xi) D99136_2F7 [MICA/B]_H1 (SEQ ID NOs: 367 and 375), (xii) D99136_2F7 [MICA/B]_H2 (SEQ ID NOs: 383 and 391), (xiii) D103388_1C7 [MICA/B]_H0 (SEQ ID NO: 399), (xiv) D103388_1C7 [MICA/B]_H1 (SEQ ID NOs: 407 and 415), (xv) D103388_1C7 [MICA/B]_H2 (SEQ ID NOs: 423 and 431), (xvi) D103388_1D7 [MICA/B]_H0 (SEQ ID NO: 439), (xvii) D103388_1D7 [MICA/B]_H1 (SEQ ID NOs: 447 and 455), (xviii) D103388_1D7 [MICA/B]_H2 (SEQ ID NOs: 463 and 471), (xix) D105317_1A2 [MICA/B]_H0 (SEQ ID NO: 479), (xx) D105317_1A2 [MICA/B]_H1 (SEQ ID NOs: 487 and 495), (xxi) D105317_1A2 [MICA/B]_H2 (SEQ ID NOs: 503 and 511), (xxii) D99136_2C11 [MICA/B]_H0 (SEQ ID NO: 519), (xxiii) D99136_2C11 [MICA/B]_H1 (SEQ ID NOs: 527 and 535), (xxiv) D99136_2C11 [MICA/B]_H2 (SEQ ID NOs: 543 and 551), (xxv) D103388_1B11-2 [MICA/B]_H0 (SEQ ID NO: 559), (xxvi) D105317_1B6 [MICA/B]_H0 (SEQ ID NO: 567), (xxvii) D105317_1C8 [MICA/B]_H0 (SEQ ID NO: 575), (xxviii) D105317_1F6 [MICA/B]_H0 (SEQ ID NO: 583), (xxix) D105317_1F7 [MICA/B]_H0 (SEQ ID NO: 591), (xxx) D94837_1D3 [MICA/B]_H0 (SEQ ID NO: 599), (xxxi) D94837_1D8 [MICA/B]_H0 (SEQ ID NO: 607), (xxxii) D94837_1D9 [MICA/B]_H0 (SEQ ID NO: 615), (xxxiii) D94837_1E1 [MICA/B]_H0 (SEQ ID NO: 623), (xxxiv) D94852_2D4 [MICA/B]_H0 (SEQ ID NO: 631), (xxxv) D94852_2G8 [MICA/B]_H0 (SEQ ID NO: 639), (xxxvi) D88487_2A8 [MICA/B]_H0 (SEQ ID NO: 647), (xxxvii) D94837_3B12 [MICA/B]_H0 (SEQ ID NO: 655), (xxxviii) D99122_1H7 [MICA/B]_H0 (SEQ ID NO: 663), (xxxix) D99136_2E8 [MICA/B]H0 (SEQ ID NO: 671), (xl) D88487_2A8 [MICA/B]_H0 (SEQ ID NO: 647), (xli) 3F9[MICA/B]_H0 (SEQ ID NO: 679), (xlii) 6E1[MICA/B]_H0 (SEQ ID NO: 687), (xliii) 7C6[MICA/B]_H0 (SEQ ID NO: 695), (xliv) 13A9 [MICA/B]_H0 (SEQ ID NO: 703), and (xlv) 1D5 [MICA/B]_H0 (SEQ ID NO: 711), as shown in FIGS. 21 and 22.

In some embodiments, the MICA/B ABD has a set of vlCDRs selected from the vlCDR1, vlCDR2, and vlCDR3 sequences from a V_{L} selected from the group including: (i) D94837_1E11_1 [MICA/B]_L0 (SEQ ID NOs: 244-246), (ii) D94837_1E11_1 [MICA/B]_L1 (SEQ ID NOs: 252-254 and 268-270), (iii) D94837_1E11_1 [MICA/B]_L2 (SEQ ID NOs: 260-262 and 276-278), (iv) 2E5 [MICA/B]_L0 (SEQ ID NOs: 284-286), (v) 2E5 [MICA/B]_L1 (SEQ ID NOs: 292-294 and 308-310), (vi) 2E5 [MICA/B]_L2 (SEQ ID NOs: 300-302 and 316-318), (vii) D94852_2E12 [MICA/B]_L0 (SEQ ID NOs: 324-326), (viii) D94852_2E12 [MICA/B]_L1 (SEQ ID NOs: 332-334 and 348-350), (ix) D94852_2E12 [MICA/B]_L2 (SEQ ID NOs: 340-342 and 356-358), (x) D99136_2F7 [MICA/B]_L0 (SEQ ID NOs: 364-366), (xi) D99136_2F7 [MICA/B]_L1 (SEQ ID NOs: 372-374 and 388-390), (xii) D99136_2F7 [MICA/B]_L2 (SEQ ID NOs: 380-382 and 396-398), (xiii) D103388_1C7 [MICA/B]_L0 (SEQ ID NOs: 404-406), (xiv) D103388_1C7 [MICA/B]_L1 (SEQ ID NOs: 412-414 and 428-430), (xv) D103388_1C7 [MICA/B]_L2 (SEQ ID NOs: 420-422 and 436-438), (xvi) D103388_1D7 [MICA/B]_L0 (SEQ ID NOs: 444-446), (xvii) D103388_1D7 [MICA/B]_L1 (SEQ ID NOs: 452-454 and 468-470), (xviii) D103388_1D7 [MICA/B]_L2 (SEQ ID NOs: 460-462 and 476-478), (xix) D105317_1A2 [MICA/B]_L0 (SEQ ID NOs: 484-486), (xx) D105317_1A2 [MICA/B]_L1 (SEQ ID NOs: 492-494 and 508-510), (xxi) D105317_1A2 [MICA/B]_L2 (SEQ ID NOs: 500-502 and 516-518), (xxii) D99136_2C11 [MICA/B]_L0 (SEQ ID NOs: 524-526), (xxiii) D99136_2C11 [MICA/B]_L1 (SEQ ID NOs: 532-534 and 548-550), (xxiv) D99136_2C11 [MICA/B]_L2 (SEQ ID NOs: 540-542 and 556-558), (xxv) D103388_1B11-2 [MICA/B]_L0 (SEQ ID NOs: 564-566), (xxvi) D105317_1B6 [MICA/B]_L0 (SEQ ID NOs: 572-574), (xxvii) D105317_1C8 [MICA/B]_L0 (SEQ ID NOs: 580-582), (xxviii) D105317_1F6 [MICA/B]_L0 (SEQ ID NOs: 588-590), (xxix) D105317_1F7 [MICA/B]_L0 (SEQ ID NOs: 596-598), (xxx) D94837_1D3 [MICA/B]_L0 (SEQ ID NOs: 604-606), (xxxi) D94837_1D8 [MICA/B]_L0 (SEQ ID NOs: 612-614), (xxxii) D94837_1D9 [MICA/B]_L0 (SEQ ID NOs: 620-622), (xxxiii) D94837_1E1 [MICA/B]_L0 (SEQ ID NOs: 628-630), (xxxiv) D94852_2D4 [MICA/B]_L0 (SEQ ID NOs: 636-638), (xxxv) D94852_2G8 [MICA/B]_L0 (SEQ ID NOs: 644-646), (xxxvi) D88487_2A8 [MICA/B]_L0 (SEQ ID NOs: 652-654), (xxxvii) D94837_3B12 [MICA/B]_L0 (SEQ ID NOs: 660-662), (xxxviii) D99122_1H7 [MICA/B]_L0 (SEQ ID NOs: 668-670), (xxxix) D99136_2E8 [MICA/B]_L0 (SEQ ID NOs: 676-678), (xl) D88487_2A8 [MICA/B]_L0 (SEQ ID NOs: 652-654), (xli) 3F9[MICA/B]_L0 (SEQ ID NOs: 684-686), (xlii) 6E1[MICA/B]_L0 (SEQ ID NOs: 692-694), (xliii) 7C6[MICA/B]_L0 (SEQ ID NOs: 700-702), (xliv) 13A9 [MICA/B]_L0 (SEQ ID NOs: 708-710), and (xlv) 1D5 [MICA/B]_L0 (SEQ ID NOs: 716-718), as shown in FIGS. 21 and 22. In some embodiments, the vlCDR1, v1CDR2, and vlCDR3 sequences of the MICA/B ABD are SEQ ID NOs: 244-246 (such as, for example, in D94837_1E11_1 [MICA/B]_L0). In other embodiments, the vlCDR1, vlCDR2, and vlCDR3 sequences of the MICA/B ABD are SEQ ID NOs: 252-254 and 268-270 (such as, for example, in D94837_1E11_1 [MICA/B]_L1). In other embodiments, the vlCDR1, vlCDR2, and vlCDR3 sequences of the MICA/B ABD are SEQ ID NOs: 260-262 and 276-278 (such as, for example, in D94837_1E11_1 [MICA/B]_L2). In other embodiments, the vlCDR1, vlCDR2, and vlCDR3 sequences of the MICA/B ABD are SEQ ID NOs: 284-286 (such as, for example, in 2E5 [MICA/B]_L0). In other embodiments, the vlCDR1, vlCDR2, and vlCDR3 sequences of the MICA/B ABD are SEQ ID NOs: 292-294 and 308-310 (such as, for example, in 2E5 [MICA/B]_L1). In other embodiments, the vlCDR1, vlCDR2, and vlCDR3 sequences of the MICA/B ABD are SEQ ID NOs: 300-302 and 316-318 (such as, for example, in 2E5 [MICA/B]_L2). In other embodiments, the vlCDR1, vlCDR2, and vlCDR3 sequences of the MICA/B ABD are SEQ ID NOs: 324-326 (such as, for example, in D94852_2E12 [MICA/B]_L0). In other embodiments, the vlCDR1, vlCDR2, and vlCDR3 sequences of the MICA/B ABD are SEQ ID NOs: 332-334 and 348-350 (such as, for example, in D94852_2E12 [MICA/B]_L1). In other embodiments, the vlCDR1, vlCDR2, and vlCDR3 sequences of the MICA/B ABD are SEQ ID NOs: 340-342 and 356-358 (such as, for example, in D94852_2E12 [MICA/B]_L2). In other embodiments, the vlCDR1, vlCDR2, and vlCDR3 sequences of the MICA/B ABD are SEQ ID NOs: 364-366 (such as, for example, in D99136_2F7 [MICA/B]_L0). In other embodiments, the vlCDR1, vlCDR2, and vlCDR3 sequences of the MICA/B ABD are SEQ ID NOs: 372-374 and 388-390 (such as, for example, in D99136_2F7 [MICA/B]_L1). In other embodiments, the vlCDR1, vlCDR2, and vlCDR3 sequences of the MICA/B ABD are SEQ ID NOs: 380-382 and 396-398 (such as, for example, in D99136_2F7 [MICA/B]_L2). In other embodiments, the vlCDR1, vlCDR2, and vlCDR3 sequences of the MICA/B ABD are SEQ ID NOs: 404-406 (such as, for example, in D103388_1C7 [MICA/B]_L0). In other embodiments, the vlCDR1, v1CDR2, and vlCDR3 sequences of the MICA/B ABD are SEQ ID NOs: 412-414 and 428-430 (such as, for example, in D103388_1C7 [MICA/B]_L1). In other embodiments, the vlCDR1, vlCDR2, and vlCDR3 sequences of the MICA/B ABD are SEQ ID NOs: 420-422 and 436-438 (such as, for example, in D103388_1C7 [MICA/B]_L2). In other embodiments, the vlCDR1, vlCDR2, and vlCDR3 sequences of the MICA/B ABD are SEQ ID NOs: 444-446 (such as, for example, in D103388_1D7 [MICA/B]_L0). In other embodiments, the vlCDR1, vlCDR2, and vlCDR3 sequences of the MICA/B ABD are SEQ ID NOs: 452-454 and 468-470 (such as, for example, in D103388_1D7 [MICA/B]_L1). In other embodiments, the vlCDR1, vlCDR2, and vlCDR3 sequences of the MICA/B ABD are SEQ ID NOs: 460-462 and 476-478 (such as, for example, in D103388_1D7 [MICA/B]_L2). In other embodiments, the vlCDR1, vlCDR2, and vlCDR3 sequences of the MICA/B ABD are SEQ ID NOs: 484-486 (such as, for example, in D105317_1A2 [MICA/B]_L0). In other embodiments, the vlCDR1, vlCDR2, and vlCDR3 sequences of the MICA/B ABD are SEQ ID NOs: 492-494 and 508-510 (such as, for example, in D105317_1A2 [MICA/B]_L1). In other embodiments, the vlCDR1, vlCDR2, and vlCDR3 sequences of the MICA/B ABD are SEQ ID NOs: 500-502 and 516-518 (such as, for example, in D105317_1A2 [MICA/B]_L2). In other embodiments, the vlCDR1, vlCDR2, and vlCDR3 sequences of the MICA/B ABD are SEQ ID NOs: 524-526 (such as, for example, in D99136_2C11 [MICA/B]_L0). In other embodiments, the vlCDR1, vlCDR2, and vlCDR3 sequences of the MICA/B ABD are SEQ ID NOs: 532-534 and 548-550 (such as, for example, in D99136_2C11 [MICA/B]_L1). In other embodiments, the vlCDR1, vlCDR2, and vlCDR3 sequences of the MICA/B ABD are SEQ ID NOs: 540-542 and 556-558 (such as, for example, in D99136_2C11 [MICA/B]_L2). In other embodiments, the vlCDR1, vlCDR2, and vlCDR3 sequences of the MICA/B ABD are SEQ ID NOs: 564-566 (such as, for example, in D103388_1B11-2 [MICA/B]_L0). In other embodiments, the vlCDR1, vlCDR2, and vlCDR3 sequences of the MICA/B ABD are SEQ ID NOs: 572-574 (such as, for example, in D105317_1B6 [MICA/B]_L0). In other embodiments, the vlCDR1, vlCDR2, and vlCDR3 sequences of the MICA/B ABD are SEQ ID NOs: 580-582 (such as, for example, in D105317_1C8 [MICA/B]_L0). In other embodiments, the vlCDR1, vlCDR2, and vlCDR3 sequences of the MICA/B ABD are SEQ ID NOs: 588-590 (such as, for example, in D105317_1F6 [MICA/B]_L0). In other embodiments, the vlCDR1, vlCDR2, and vlCDR3 sequences of the MICA/B ABD are SEQ ID NOs: 596-598 (such as, for example, in D105317_1F7 [MICA/B]_L0). In other embodiments, the vlCDR1, vlCDR2, and vlCDR3 sequences of the MICA/B ABD are SEQ ID NOs: 604-606 (such as, for example, in D94837_1D3 [MICA/B]_L0). In other embodiments, the vlCDR1, vlCDR2, and vlCDR3 sequences of the MICA/B ABD are SEQ ID NOs: 612-614 (such as, for example, in D94837_1D8 [MICA/B]_L0). In other embodiments, the vlCDR1, vlCDR2, and vlCDR3 sequences of the MICA/B ABD are SEQ ID NOs: 620-622 (such as, for example, in D94837_1D9 [MICA/B]_L0). In other embodiments, the vlCDR1, vlCDR2, and vlCDR3 sequences of the MICA/B ABD are SEQ ID NOs: 628-630 (such as, for example, in D94837_1E1 [MICA/B]_L0). In other embodiments, the vlCDR1, vlCDR2, and vlCDR3 sequences of the MICA/B ABD are SEQ ID NOs: 636-638 (such as, for example, in D94852_2D4 [MICA/B]_L0). In other embodiments, the vlCDR1, vlCDR2, and vlCDR3 sequences of the MICA/B ABD are SEQ ID NOs: 644-646 (such as, for example, in D94852_2G8 [MICA/B]_L0). In other embodiments, the vlCDR1, vlCDR2, and vlCDR3 sequences of the MICA/B ABD are SEQ ID NOs: 652-654 (such as, for example, in D88487_2A8 [MICA/B]_L0). In other embodiments, the vlCDR1, vlCDR2, and vlCDR3 sequences of the MICA/B ABD are SEQ ID NOs: 660-662 (such as, for example, in D94837_3B12 [MICA/B]_L0). In other embodiments, the vlCDR1, vlCDR2, and vlCDR3 sequences of the MICA/B ABD are SEQ ID NOs: 668-670 (such as, for example, in D99122_1H7 [MICA/B]_L0). In other embodiments, the vlCDR1, vlCDR2, and vlCDR3 sequences of the MICA/B ABD are SEQ ID NOs: 676-678 (such as, for example, in D99136_2E8 [MICA/B]_L0). In other embodiments, the vlCDR1, vlCDR2, and vlCDR3 sequences of the MICA/B ABD are SEQ ID NOs: 652-654 (such as, for example, in D88487_2A8 [MICA/B]_L0). In other embodiments, the vlCDR1, vlCDR2, and vlCDR3 sequences of the MICA/B ABD are SEQ ID NOs: 684-686 (such as, for example, in 3F9[MICA/B]_L0). In other embodiments, the vlCDR1, vlCDR2, and vlCDR3 sequences of the MICA/B ABD are SEQ ID NOs: 692-694 (such as, for example, in 6E1[MICA/B]_L0). In other embodiments, the vlCDR1, vlCDR2, and vlCDR3 sequences of the MICA/B ABD are SEQ ID NOs: 700-702 (such as, for example, in 7C6[MICA/B]_L0). In other embodiments, the vlCDR1, vlCDR2, and vlCDR3 sequences of the MICA/B ABD are SEQ ID NOs: 708-710 (such as, for example, in 13A9 [MICA/B]_L0). In other embodiments, the vlCDR1, vlCDR2, and vlCDR3 sequences of the MICA/B ABD are SEQ ID NOs: 716-718 (such as, for example, in 1D5 [MICA/B]_L0).

In some embodiments, the V_{L} of the MICA/B ABD is selected from the group including: (i) D94837_1E11_1 [MICA/B]_L0 (SEQ ID NO: 243), (ii) D94837_1E11_1 [MICA/B]_L1 (SEQ ID NOs: 251 and 267), (iii) D94837_1E11_1 [MICA/B]_L2 (SEQ ID NOs: 259 and 275), (iv) 2E5 [MICA/B]_L0 (SEQ ID NO: 283), (v) 2E5 [MICA/B]_L1 (SEQ ID NOs: 291 and 307), (vi) 2E5 [MICA/B]_L2 (SEQ ID NOs: 299 and 315), (vii) D94852_2E12 [MICA/B]_L0 (SEQ ID NO: 323), (viii) D94852_2E12 [MICA/B]_L1 (SEQ ID NOs: 331 and 347), (ix) D94852_2E12 [MICA/B]_L2 (SEQ ID NOs: 339 and 355), (x) D99136_2F7 [MICA/B]_L0 (SEQ ID NO: 363), (xi) D99136_2F7 [MICA/B]_L1 (SEQ ID NOs: 371 and 387), (xii) D99136_2F7 [MICA/B]_L2 (SEQ ID NOs: 379 and 395), (xiii) D103388_1C7 [MICA/B]_L0 (SEQ ID NO: 403), (xiv) D103388_1C7 [MICA/B]_L1 (SEQ ID NOs: 411 and 427), (xv) D103388_1C7 [MICA/B]_L2 (SEQ ID NOs: 419 and 435), (xvi) D103388_1D7 [MICA/B]_L0 (SEQ ID NO: 443), (xvii) D103388_1D7 [MICA/B]_L1 (SEQ ID NOs: 451 and 467), (xviii) D103388_1D7 [MICA/B]_L2 (SEQ ID NOs: 459 and 475), (xix) D105317_1A2 [MICA/B]_L0 (SEQ ID NO: 483), (xx) D105317_1A2 [MICA/B]_L1 (SEQ ID NOs: 491 and 507), (xxi) D105317_1A2 [MICA/B]_L2 (SEQ ID NOs: 499 and 515), (xxii) D99136_2C11 [MICA/B]_L0 (SEQ ID NO: 523), (xxiii) D99136_2C11 [MICA/B]_L1 (SEQ ID NOs: 531 and 547), (xxiv) D99136_2C11 [MICA/B]_L2 (SEQ ID NOs: 539 and 555), (xxv) D103388_1B11-2 [MICA/B]_L0 (SEQ ID NO: 563), (xxvi) D105317_1B6 [MICA/B]_L0 (SEQ ID NO: 571), (xxvii) D105317_1C8 [MICA/B]_L0 (SEQ ID NO: 579), (xxviii) D105317_1F6 [MICA/B]_L0 (SEQ ID NO: 587), (xxix) D105317_1F7 [MICA/B]_L0 (SEQ ID NO: 595), (xxx) D94837_1D3 [MICA/B]_L0 (SEQ ID NO: 603), (xxxi) D94837_1D8 [MICA/B]_L0 (SEQ ID NO: 611), (xxxii) D94837_1D9 [MICA/B]_L0 (SEQ ID NO: 619), (xxxiii) D94837_1E1 [MICA/B]_L0 (SEQ ID NO: 627), (xxxiv) D94852_2D4 [MICA/B]_L0 (SEQ ID NO: 635), (xxxv) D94852_2G8 [MICA/B]_L0 (SEQ ID NO: 643), (xxxvi) D88487_2A8 [MICA/B]_L0 (SEQ ID NO: 651), (xxxvii) D94837_3B12 [MICA/B]_L0 (SEQ ID NO: 659), (xxxviii) D99122_1H7 [MICA/B]_L0 (SEQ ID NO: 667), (xxxix) D99136_2E8 [MICA/B]_L0 (SEQ ID NO: 675), (xl) D88487_2A8 [MICA/B]_L0 (SEQ ID NO: 651), (xli) 3F9[MICA/B]_L0 (SEQ ID NO: 683), (xlii) 6E1[MICA/B]_L0 (SEQ ID NO: 691), (xliii) 7C6[MICA/B]_L0 (SEQ ID NO: 699), (xliv) 13A9 [MICA/B]_L0 (SEQ ID NO: 707), and (xlv) 1D5 [MICA/B]_L0 (SEQ ID NO: 715), as shown in FIGS. 21 and 22.

Accordingly, included herein are MICA/B ABDs that have a set of 6 CDRs (i.e., vhCDR1-3 and vlCDR1-3) from V_{H}/V_{L} pairs selected from the group including: (i) SEQ ID NOs: 240-242 for vhCDR1-3 and SEQ ID NOs: 244-246 for vlCDR1-3 of D94837_1E11_1 [MICA/B]_H0_D94837_1E11_1 [MICA/B]_L0, respectively, (ii) SEQ ID NOs: 248-250 for vhCDR1-3 and SEQ ID NOs: 252-254 for vlCDR1-3 of D94837_1E11_1 [MICA/B]_H1_D94837_1E11_1 [MICA/B]_L1, respectively, (iii) SEQ ID NOs: 256-258 for vhCDR1-3 and SEQ ID NOs: 260-262 for vlCDR1-3 of D94837_1E11_1 [MICA/B]_H1_D94837_E11_1 [MICA/B]_L2, respectively, (iv) SEQ ID NOs: 264-266 for vhCDR1-3 and SEQ ID NOs: 268-270 for vlCDR1-3 of D94837_1E11_1 [MICA/B]_H2_D94837_1E11_1 [MICA/B]_L1, respectively, (v) SEQ ID NOs: 272-274 for vhCDR1-3 and SEQ ID NOs: 276-278 for vlCDR1-3 of D94837_1E11_1 [MICA/B]_H2_D94837_E11_1 [MICA/B]_L2, respectively, (vi) SEQ ID NOs: 280-282 for vhCDR1-3 and SEQ ID NOs: 284-286 for vlCDR1-3 of 2E5 [MICA/B]_H0_2E5 [MICA/B]_L0, respectively, (vii) SEQ ID NOs: 288-290 for vhCDR1-3 and SEQ ID NOs: 292-294 for vlCDR1-3 of 2E5 [MICA/B]_H1_2E5 [MICA/B]_L1, respectively, (viii) SEQ ID NOs: 296-298 for vhCDR1-3 and SEQ ID NOs: 300-302 for vlCDR1-3 of 2E5 [MICA/B]_H1_2E5 [MICA/B]_L2, respectively, (ix) SEQ ID NOs: 304-306 for vhCDR1-3 and SEQ ID NOs: 308-310 for vlCDR1-3 of 2E5 [MICA/B]_H2_2E5 [MICA/B]_L1, respectively, (x) SEQ ID NOs: 312-314 for vhCDR1-3 and SEQ ID NOs: 316-318 for vlCDR1-3 of 2E5 [MICA/B]_H2_2E5 [MICA/B]_L2, respectively, (xi) SEQ ID NOs: 320-322 for vhCDR1-3 and SEQ ID NOs: 324-326 for vlCDR1-3 of D94852_2E12 [MICA/B]_H0_D94852_2E12 [MICA/B]_L0, respectively, (xii) SEQ ID NOs: 328-330 for vhCDR1-3 and SEQ ID NOs: 332-334 for vlCDR1-3 of D94852_2E12 [MICA/B]_H1_D94852_2E12 [MICA/B]_L1, respectively, (xiii) SEQ ID NOs: 336-338 for vhCDR1-3 and SEQ ID NOs: 340-342 for vlCDR1-3 of D94852_2E12 [MICA/B]_H1_D94852_2E12 [MICA/B]_L2, respectively, (xiv) SEQ ID NOs: 344-346 for vhCDR1-3 and SEQ ID NOs: 348-350 for vlCDR1-3 of D94852_2E12 [MICA/B]_H2_D94852_2E12 [MICA/B]_L1, respectively, (xv) SEQ ID NOs: 352-354 for vhCDR1-3 and SEQ ID NOs: 356-358 for vlCDR1-3 of D94852_2E12 [MICA/B]_H2_D94852_2E12 [MICA/B]_L2, respectively, (xvi) SEQ ID NOs: 360-362 for vhCDR1-3 and SEQ ID NOs: 364-366 for vlCDR1-3 of D99136_2F7 [MICA/B]_H0_D99136_2F7 [MICA/B]_L0, respectively, (xvii) SEQ ID NOs: 368-370 for vhCDR1-3 and SEQ ID NOs: 372-374 for vlCDR1-3 of D99136_2F7 [MICA/B]_H1_D99136_2F7 [MICA/B]_L1, respectively, (xviii) SEQ ID NOs: 376-378 for vhCDR1-3 and SEQ ID NOs: 380-382 for vlCDR1-3 of D99136_2F7 [MICA/B]_H1_D99136_2F7 [MICA/B]_L2, respectively, (xix) SEQ ID NOs: 384-386 for vhCDR1-3 and SEQ ID NOs: 388-390 for vlCDR1-3 of D99136_2F7 [MICA/B]_H2_D99136_2F7 [MICA/B]_L1, respectively, (xx) SEQ ID NOs: 392-394 for vhCDR1-3 and SEQ ID NOs: 396-398 for vlCDR1-3 of D99136_2F7 [MICA/B]_H2_D99136_2F7 [MICA/B]_L2, respectively, (xxi) SEQ ID NOs: 400-402 for vhCDR1-3 and SEQ ID NOs: 404-406 for vlCDR1-3 of D103388_1C7 [MICA/B]_H0_D103388_1C7 [MICA/B]_L0, respectively, (xxii) SEQ ID NOs: 408-410 for vhCDR1-3 and SEQ ID NOs: 412-414 for vlCDR1-3 of D103388_1C7 [MICA/B]_H1_D103388_1C7 [MICA/B]_L1, respectively, (xxiii) SEQ ID NOs: 416-418 for vhCDR1-3 and SEQ ID NOs: 420-422 for vlCDR1-3 of D103388_1C7 [MICA/B]_H1_D103388_1C7 [MICA/B]_L2, respectively, (xxiv) SEQ ID NOs: 424-426 for vhCDR1-3 and SEQ ID NOs: 428-430 for vlCDR1-3 of D103388_1C7 [MICA/B]_H2_D103388_1C7 [MICA/B]_L1, respectively, (xxv) SEQ ID NOs: 432-434 for vhCDR1-3 and SEQ ID NOs: 436-438 for vlCDR1-3 of D103388_1C7 [MICA/B]_H2_D103388_1C7 [MICA/B]_L2, respectively, (xxvi) SEQ ID NOs: 440-442 for vhCDR1-3 and SEQ ID NOs: 444-446 for vlCDR1-3 of D103388_1D7 [MICA/B]_H0_D103388_1D7 [MICA/B]_L0, respectively, (xxvii) SEQ ID NOs: 448-450 for vhCDR1-3 and SEQ ID NOs: 452-454 for vlCDR1-3 of D103388_1D7 [MICA/B]_H1_D103388_1D7 [MICA/B]_L1, respectively, (xxviii) SEQ ID NOs: 456-458 for vhCDR1-3 and SEQ ID NOs: 460-462 for vlCDR1-3 of D103388_1D7 [MICA/B]_H1_D103388_1D7 [MICA/B]_L2, respectively, (xxix) SEQ ID NOs: 464-466 for vhCDR1-3 and SEQ ID NOs: 468-470 for vlCDR1-3 of D103388_1D7 [MICA/B]_H2_D103388_1D7 [MICA/B]_L1, respectively, (xxx) SEQ ID NOs: 472-474 for vhCDR1-3 and SEQ ID NOs: 476-478 for vlCDR1-3 of D103388_1D7 [MICA/B]_H2_D103388_1D7 [MICA/B]_L2, respectively, (xxxi) SEQ ID NOs: 480-482 for vhCDR1-3 and SEQ ID NOs: 484-486 for vlCDR1-3 of D105317_1A2 [MICA/B]_H0_D105317_1A2 [MICA/B]_L0, respectively, (xxxii) SEQ ID NOs: 488-490 for vhCDR1-3 and SEQ ID NOs: 492-494 for vlCDR1-3 of D105317_1A2 [MICA/B]_H1_D105317_1A2 [MICA/B]_L1, respectively, (xxxiii) SEQ ID NOs: 496-498 for vhCDR1-3 and SEQ ID NOs: 500-502 for vlCDR1-3 of D105317_1A2 [MICA/B]_H1_D105317_1A2 [MICA/B]_L2, respectively, (xxxiv) SEQ ID NOs: 504-506 for vhCDR1-3 and SEQ ID NOs: 508-510 for vlCDR1-3 of D105317_1A2 [MICA/B]_H2_D105317_1A2 [MICA/B]_L1, respectively, (xxxv) SEQ ID NOs: 512-514 for vhCDR1-3 and SEQ ID NOs: 516-518 for vlCDR1-3 of D105317_1A2 [MICA/B]_H2_D105317_1A2 [MICA/B]_L2, respectively, (xxxvi) SEQ ID NOs: 520-522 for vhCDR1-3 and SEQ ID NOs: 524-526 for vlCDR1-3 of D99136_2C11 [MICA/B]_H0_D99136_2C11 [MICA/B]_L0, respectively, (xxxvii) SEQ ID NOs: 528-530 for vhCDR1-3 and SEQ ID NOs: 532-534 for vlCDR1-3 of D99136_2C11 [MICA/B]_H1_D99136_2C11 [MICA/B]_L1, respectively, (xxxviii) SEQ ID NOs: 536-538 for vhCDR1-3 and SEQ ID NOs: 540-542 for vlCDR1-3 of D99136_2C11 [MICA/B]_H1_D99136_2C11 [MICA/B]_L2, respectively, (xxxix) SEQ ID NOs: 544-546 for vhCDR1-3 and SEQ ID NOs: 548-550 for vlCDR1-3 of D99136_2C11 [MICA/B]_H2_D99136_2C11 [MICA/B]_L1, respectively, (xl) SEQ ID NOs: 552-554 for vhCDR1-3 and SEQ ID NOs: 556-558 for vlCDR1-3 of D99136_2C11 [MICA/B]_H2_D99136_2C11 [MICA/B]_L2, respectively, (xli) SEQ ID NOs: 560-562 for vhCDR1-3 and SEQ ID NOs: 564-566 for vlCDR1-3 of D103388_1B11-2 [MICA/B]_H0_D103388_1B11-2 [MICA/B]_L0, respectively, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, respectively, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, respectively, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, respectively, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, respectively, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, respectively, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, respectively, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, respectively, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, respectively, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, respectively, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, respectively, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, respectively, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, respectively, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, respectively, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, respectively, (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, respectively, (lvii) SEQ ID NOs: 680-682 for vhCDR1-3 and SEQ ID NOs: 684-686 for vlCDR1-3 of 3F9[MICA/B]_H0_3F9[MICA/B]_L0, respectively, (lviii) SEQ ID NOs: 688-690 for vhCDR1-3 and SEQ ID NOs: 692-694 for vlCDR1-3 of 6E1[MICA/B]_H0_6E1[MICA/B]_L0, respectively, (lix) SEQ ID NOs: 696-698 for vhCDR1-3 and SEQ ID NOs: 700-702 for vlCDR1-3 of 7C6[MICA/B]_H0_7C6[MICA/B]_L0, respectively, (lx) SEQ ID NOs: 704-706 for vhCDR1-3 and SEQ ID NOs: 708-710 for vlCDR1-3 of 13A9 [MICA/B]_H0_13A9 [MICA/B]_L0, respectively, and (lxi) SEQ ID NOs: 712-714 for vhCDR1-3 and SEQ ID NOs: 716-718 for vlCDR1-3 of 1D5 [MICA/B]_H0_1D5 [MICA/B]_L0, respectively, as are generally shown in FIGS. 21 and 22.

Additionally, included herein are MICA/B ABDs that have V_{H}/V_{L} pairs selected from the group including: (i) SEQ ID Nos: 239 and 243 for D94837_1E11_1 [MICA/B]_H0_D94837_1E11_1 [MICA/B]_L0, respectively, (ii) SEQ ID Nos: 247 and 251 for D94837_1E1_1 [MICA/B]_H1_D94837_1E1_1 [MICA/B]_L1, respectively, (iii) SEQ ID Nos: 255 and 259 for D94837_E11_1 [MICA/B]_H1_D94837_1E1_1 [MICA/B]_L2, respectively, (iv) SEQ ID Nos: 263 and 267 for D94837_1E11_1 [MICA/B]_H2_D94837_1E11_1 [MICA/B]_L1, respectively, (v) SEQ ID Nos: 271 and 275 for D94837_1E11_1 [MICA/B]_H2_D94837_1E11_1 [MICA/B]_L2, respectively, (vi) SEQ ID Nos: 279 and 283 for 2E5 [MICA/B]_H0_2E5 [MICA/B]_L0, respectively, (vii) SEQ ID Nos: 287 and 291 for 2E5 [MICA/B]_H1_2E5 [MICA/B]_L1, respectively, (viii) SEQ ID Nos: 295 and 299 for 2E5 [MICA/B]_H1_2E5 [MICA/B]_L2, respectively, (ix) SEQ ID Nos: 303 and 307 for 2E5 [MICA/B]_H2_2E5 [MICA/B]_L1, respectively, (x) SEQ ID Nos: 311 and 315 for 2E5 [MICA/B]_H2_2E5 [MICA/B]_L2, respectively, (xi) SEQ ID Nos: 319 and 323 for D94852_2E12 [MICA/B]_H0_D94852_2E12 [MICA/B]_L0, respectively, (xii) SEQ ID Nos: 327 and 331 for D94852_2E12 [MICA/B]_H1_D94852_2E12 [MICA/B]_L1, respectively, (xiii) SEQ ID Nos: 335 and 339 for D94852_2E12 [MICA/B]_H1_D94852_2E12 [MICA/B]_L2, respectively, (xiv) SEQ ID Nos: 343 and 347 for D94852_2E12 [MICA/B]_H2_D94852_2E12 [MICA/B]_L1, respectively, (xv) SEQ ID Nos: 351 and 355 for D94852_2E12 [MICA/B]_H2_D94852_2E12 [MICA/B]_L2, respectively, (xvi) SEQ ID Nos: 359 and 363 for D99136_2F7 [MICA/B]_H0_D99136_2F7 [MICA/B]_L0, respectively, (xvii) SEQ ID Nos: 367 and 371 for D99136_2F7 [MICA/B]_H1_D99136_2F7 [MICA/B]_L1, respectively, (xviii) SEQ ID Nos: 375 and 379 for D99136_2F7 [MICA/B]_H1_D99136_2F7 [MICA/B]_L2, respectively, (xix) SEQ ID Nos: 383 and 387 for D99136_2F7 [MICA/B]_H2_D99136_2F7 [MICA/B]_L1, respectively, (xx) SEQ ID Nos: 391 and 395 for D99136_2F7 [MICA/B]_H2_D99136_2F7 [MICA/B]_L2, respectively, (xxi) SEQ ID Nos: 399 and 403 for D103388_1C7 [MICA/B]_H0_D103388_1C7 [MICA/B]_L0, respectively, (xxii) SEQ ID Nos: 407 and 411 for D103388_1C7 [MICA/B]_H1_D103388_1C7 [MICA/B]_L1, respectively, (xxiii) SEQ ID Nos: 415 and 419 for D103388_1C7 [MICA/B]_H1_D103388_1C7 [MICA/B]_L2, respectively, (xxiv) SEQ ID Nos: 423 and 427 for D103388_1C7 [MICA/B]_H2_D103388_1C7 [MICA/B]_L1, respectively, (xxv) SEQ ID Nos: 431 and 435 for D103388_1C7 [MICA/B]_H2_D103388_1C7 [MICA/B]_L2, respectively, (xxvi) SEQ ID Nos: 439 and 443 for D103388_1D7 [MICA/B]_H0_D103388_1D7 [MICA/B]_L0, respectively, (xxvii) SEQ ID Nos: 447 and 451 for D103388_1D7 [MICA/B]_H1_D103388_1D7 [MICA/B]_L1, respectively, (xxviii) SEQ ID Nos: 455 and 459 for D103388_1D7 [MICA/B]_H1_D103388_1D7 [MICA/B]_L2, respectively, (xxix) SEQ ID Nos: 463 and 467 for D103388_1D7 [MICA/B]_H2_D103388_1D7 [MICA/B]_L1, respectively, (xxx) SEQ ID Nos: 471 and 475 for D103388_1D7 [MICA/B]_H2_D103388_1D7 [MICA/B]_L2, respectively, (xxxi) SEQ ID Nos: 479 and 483 for D105317_1A2 [MICA/B]_H0_D105317_1A2 [MICA/B]_L0, respectively, (xxxii) SEQ ID Nos: 487 and 491 for D105317_1A2 [MICA/B]_H1_D105317_1A2 [MICA/B]_L1, respectively, (xxxiii) SEQ ID Nos: 495 and 499 for D105317_1A2 [MICA/B]_H1_D105317_1A2 [MICA/B]_L2, respectively, (xxxiv) SEQ ID Nos: 503 and 507 for D105317_1A2 [MICA/B]_H2_D105317_1A2 [MICA/B]_L1, respectively, (xxxv) SEQ ID Nos: 511 and 515 for D105317_1A2 [MICA/B]_H2_D105317_1A2 [MICA/B]_L2, respectively, (xxxvi) SEQ ID Nos: 519 and 523 for D99136_2C11 [MICA/B]_H0_D99136_2C11 [MICA/B]_L0, respectively, (xxxvii) SEQ ID Nos: 527 and 531 for D99136_2C11 [MICA/B]_H1_D99136_2C11 [MICA/B]_L1, respectively, (xxxviii) SEQ ID Nos: 535 and 539 for D99136_2C11 [MICA/B]_H1_D99136_2C11 [MICA/B]_L2, respectively, (xxxix) SEQ ID Nos: 543 and 547 for D99136_2C11 [MICA/B]_H2_D99136_2C11 [MICA/B]_L1, respectively, (xl) SEQ ID Nos: 551 and 555 for D99136_2C11 [MICA/B]_H2_D99136_2C11 [MICA/B]_L2, respectively, (xli) SEQ ID Nos: 559 and 563 for D103388_1B11-2 [MICA/B]_H0_D103388_1B11-2 [MICA/B]_L0, respectively, (xlii) SEQ ID Nos: 567 and 571 for D105317_1B6 [MICA/B]_H0_D105317_1B6 [MICA/B]_L0, respectively, (xliii) SEQ ID Nos: 575 and 579 for D105317_1C8 [MICA/B]_H0_D105317_1C8 [MICA/B]_L0, respectively, (xliv) SEQ ID Nos: 583 and 587 for D105317_1F6 [MICA/B]_H0_D105317_1F6 [MICA/B]_L0, respectively, (xlv) SEQ ID Nos: 591 and 595 for D105317_1F7 [MICA/B]_H0_D105317_1F7 [MICA/B]_L0, respectively, (xlvi) SEQ ID Nos: 599 and 603 for D94837_1D3 [MICA/B]_H0_D94837_1D3 [MICA/B]_L0, respectively, (xlvii) SEQ ID Nos: 607 and 611 for D94837_1D8 [MICA/B]_H0_D94837_1D8 [MICA/B]_L0, respectively, (xlviii) SEQ ID Nos: 615 and 619 for D94837_1D9 [MICA/B]_H0_D94837_1D9 [MICA/B]_L0, respectively, (xlix) SEQ ID Nos: 623 and 627 for D94837_1E1 [MICA/B]_H0_D94837_1E1 [MICA/B]_L0, respectively, (1) SEQ ID Nos: 631 and 635 for D94852_2D4 [MICA/B]_H0_D94852_2D4 [MICA/B]_L0, respectively, (li) SEQ ID Nos: 639 and 643 for D94852_2G8 [MICA/B]_H0_D94852_2G8 [MICA/B]_L0, respectively, (lii) SEQ ID Nos: 647 and 651 for D88487_2A8 [MICA/B]_H0_D88487_2A8 [MICA/B]_L0, respectively, (liii) SEQ ID Nos: 655 and 659 for D94837_3B12 [MICA/B]_H0_D94837_3B12 [MICA/B]_L0, respectively, (liv) SEQ ID Nos: 663 and 667 for D99122_1H7 [MICA/B]_H0_D99122_1H7 [MICA/B]_L0, respectively, (lv) SEQ ID Nos: 671 and 675 for D99136_2E8 [MICA/B]_H0_D99136_2E8 [MICA/B]_L0, respectively, (lvi) SEQ ID Nos: 647 and 651 for D88487_2A8 [MICA/B]_H0_D88487_2A8 [MICA/B]_L0, respectively, (lvii) SEQ ID Nos: 679 and 683 for 3F9[MICA/B]_H0_3F9[MICA/B]_L0, respectively, (lviii) SEQ ID Nos: 687 and 691 for 6E1[MICA/B]_H0_6E1[MICA/B]_L0, respectively, (lix) SEQ ID Nos: 695 and 699 for 7C6[MICA/B]_H0_7C6[MICA/B]_L0, respectively, (lx) SEQ ID Nos: 703 and 707 for 13A9 [MICA/B]_H0_13A9 [MICA/B]_L0, respectively, and (lxi) SEQ ID Nos: 711 and 715 for 1D5 [MICA/B]_H0_1D5 [MICA/B]_L0, respectively, as are generally shown in FIGS. 21 and 22.

In particular embodiments, the V_{H}/V_{L} pairs used in scFvs are selected from the group including: (i) SEQ ID Nos: 239 and 243 for D94837_E11_1 [MICA/B]_H0_D94837_1E11_1 [MICA/B]_L0, respectively, (ii) SEQ ID Nos: 247 and 251 for D94837_1E11_1 [MICA/B]_H1_D94837_1E11_1 [MICA/B]_L1, respectively, (iii) SEQ ID Nos: 255 and 259 for D94837_1E11_1 [MICA/B]_H1_D94837_1E11_1 [MICA/B]_L2, respectively, (iv) SEQ ID Nos: 263 and 267 for D94837_E11_1 [MICA/B]_H2_D94837_E11_1 [MICA/B]_L1, respectively, (v) SEQ ID Nos: 271 and 275 for D94837_1E11_1 [MICA/B]_H2_D94837_E11_1 [MICA/B]_L2, respectively, (vi) SEQ ID Nos: 279 and 283 for 2E5 [MICA/B]_H0_2E5 [MICA/B]_L0, respectively, (vii) SEQ ID Nos: 287 and 291 for 2E5 [MICA/B]_H1_2E5 [MICA/B]_L1, respectively, (viii) SEQ ID Nos: 295 and 299 for 2E5 [MICA/B]_H1_2E5 [MICA/B]_L2, respectively, (ix) SEQ ID Nos: 303 and 307 for 2E5 [MICA/B]_H2_2E5 [MICA/B]_L1, respectively, (x) SEQ ID Nos: 311 and 315 for 2E5 [MICA/B]_H2_2E5 [MICA/B]_L2, respectively, (xi) SEQ ID Nos: 319 and 323 for D94852_2E12 [MICA/B]_H0_D94852_2E12 [MICA/B]_L0, respectively, (xii) SEQ ID Nos: 327 and 331 for D94852_2E12 [MICA/B]_H1_D94852_2E12 [MICA/B]_L1, respectively, (xiii) SEQ ID Nos: 335 and 339 for D94852_2E12 [MICA/B]_H1_D94852_2E12 [MICA/B]_L2, respectively, (xiv) SEQ ID Nos: 343 and 347 for D94852_2E12 [MICA/B]_H2_D94852_2E12 [MICA/B]_L1, respectively, (xv) SEQ ID Nos: 351 and 355 for D94852_2E12 [MICA/B]_H2_D94852_2E12 [MICA/B]_L2, respectively, (xvi) SEQ ID Nos: 359 and 363 for D99136_2F7 [MICA/B]_H0_D99136_2F7 [MICA/B]_L0, respectively, (xvii) SEQ ID Nos: 367 and 371 for D99136_2F7 [MICA/B]_H1_D99136_2F7 [MICA/B]_L1, respectively, (xviii) SEQ ID Nos: 375 and 379 for D99136_2F7 [MICA/B]_H1_D99136_2F7 [MICA/B]_L2, respectively, (xix) SEQ ID Nos: 383 and 387 for D99136_2F7 [MICA/B]_H2_D99136_2F7 [MICA/B]_L1, respectively, (xx) SEQ ID Nos: 391 and 395 for D99136_2F7 [MICA/B]_H2_D99136_2F7 [MICA/B]_L2, respectively, (xxi) SEQ ID Nos: 399 and 403 for D103388_1C7 [MICA/B]_H0_D103388_1C7 [MICA/B]_L0, respectively, (xxii) SEQ ID Nos: 407 and 411 for D103388_1C7 [MICA/B]_H1_D103388_1C7 [MICA/B]_L1, respectively, (xxiii) SEQ ID Nos: 415 and 419 for D103388_1C7 [MICA/B]_H1_D103388_1C7 [MICA/B]_L2, respectively, (xxiv) SEQ ID Nos: 423 and 427 for D103388_1C7 [MICA/B]_H2_D103388_1C7 [MICA/B]_L1, respectively, (xxv) SEQ ID Nos: 431 and 435 for D103388_1C7 [MICA/B]_H2_D103388_1C7 [MICA/B]_L2, respectively, (xxvi) SEQ ID Nos: 439 and 443 for D103388_1D7 [MICA/B]_H0_D103388_1D7 [MICA/B]_L0, respectively, (xxvii) SEQ ID Nos: 447 and 451 for D103388_1D7 [MICA/B]_H1_D103388_1D7 [MICA/B]_L1, respectively, (xxviii) SEQ ID Nos: 455 and 459 for D103388_1D7 [MICA/B]_H1_D103388_1D7 [MICA/B]_L2, respectively, (xxix) SEQ ID Nos: 463 and 467 for D103388_1D7 [MICA/B]_H2_D103388_1D7 [MICA/B]_L1, respectively, (xxx) SEQ ID Nos: 471 and 475 for D103388_1D7 [MICA/B]_H2_D103388_1D7 [MICA/B]_L2, respectively, (xxxi) SEQ ID Nos: 479 and 483 for D105317_1A2 [MICA/B]_H0_D105317_1A2 [MICA/B]_L0, respectively, (xxxii) SEQ ID Nos: 487 and 491 for D105317_1A2 [MICA/B]_H1_D105317_1A2 [MICA/B]_L1, respectively, (xxxiii) SEQ ID Nos: 495 and 499 for D105317_1A2 [MICA/B]_H1_D105317_1A2 [MICA/B]_L2, respectively, (xxxiv) SEQ ID Nos: 503 and 507 for D105317_1A2 [MICA/B]_H2_D105317_1A2 [MICA/B]_L1, respectively, (xxxv) SEQ ID Nos: 511 and 515 for D105317_1A2 [MICA/B]_H2_D105317_1A2 [MICA/B]_L2, respectively, (xxxvi) SEQ ID Nos: 519 and 523 for D99136_2C11 [MICA/B]_H0_D99136_2C11 [MICA/B]_L0, respectively, (xxxvii) SEQ ID Nos: 527 and 531 for D99136_2C11 [MICA/B]_H1_D99136_2C11 [MICA/B]_L1, respectively, (xxxviii) SEQ ID Nos: 535 and 539 for D99136_2C11 [MICA/B]_H1_D99136_2C11 [MICA/B]_L2, respectively, (xxxix) SEQ ID Nos: 543 and 547 for D99136_2C11 [MICA/B]_H2_D99136_2C11 [MICA/B]_L1, respectively, (xl) SEQ ID Nos: 551 and 555 for D99136_2C11 [MICA/B]_H2_D99136_2C11 [MICA/B]_L2, respectively, (xli) SEQ ID Nos: 559 and 563 for D103388_1B11-2 [MICA/B]_H0_D103388_1B11-2 [MICA/B]_L0, respectively, (xlii) SEQ ID Nos: 567 and 571 for D105317_1B6 [MICA/B]_H0_D105317_1B6 [MICA/B]_L0, respectively, (xliii) SEQ ID Nos: 575 and 579 for D105317_1C8 [MICA/B]_H0_D105317_1C8 [MICA/B]_L0, respectively, (xliv) SEQ ID Nos: 583 and 587 for D105317_1F6 [MICA/B]_H0_D105317_1F6 [MICA/B]_L0, respectively, (xlv) SEQ ID Nos: 591 and 595 for D105317_1F7 [MICA/B]_H0_D105317_1F7 [MICA/B]_L0, respectively, (xlvi) SEQ ID Nos: 599 and 603 for D94837_1D3 [MICA/B]_H0_D94837_1D3 [MICA/B]_L0, respectively, (xlvii) SEQ ID Nos: 607 and 611 for D94837_1D8 [MICA/B]_H0_D94837_1D8 [MICA/B]_L0, respectively, (xlviii) SEQ ID Nos: 615 and 619 for D94837_1D9 [MICA/B]_H0_D94837_1D9 [MICA/B]_L0, respectively, (xlix) SEQ ID Nos: 623 and 627 for D94837_1E1 [MICA/B]_H0_D94837_1E1 [MICA/B]_L0, respectively, (1) SEQ ID Nos: 631 and 635 for D94852_2D4 [MICA/B]_H0_D94852_2D4 [MICA/B]_L0, respectively, (li) SEQ ID Nos: 639 and 643 for D94852_2G8 [MICA/B]_H0_D94852_2G8 [MICA/B]_L0, respectively, (lii) SEQ ID Nos: 647 and 651 for D88487_2A8 [MICA/B]_H0_D88487_2A8 [MICA/B]_L0, respectively, (liii) SEQ ID Nos: 655 and 659 for D94837_3B12 [MICA/B]_H0_D94837_3B12 [MICA/B]_L0, respectively, (liv) SEQ ID Nos: 663 and 667 for D99122_1H7 [MICA/B]_H0_D99122_1H7 [MICA/B]_L0, respectively, (lv) SEQ ID Nos: 671 and 675 for D99136_2E8 [MICA/B]_H0_D99136_2E8 [MICA/B]_L0, respectively, (lvi) SEQ ID Nos: 647 and 651 for D88487_2A8 [MICA/B]_H0_D88487_2A8 [MICA/B]_L0, respectively, (lvii) SEQ ID Nos: 679 and 683 for 3F9[MICA/B]_H0_3F9[MICA/B]_L0, respectively, (lviii) SEQ ID Nos: 687 and 691 for 6E1[MICA/B]_H0_6E1[MICA/B]_L0, respectively, (lix) SEQ ID Nos: 695 and 699 for 7C6[MICA/B]_H0_7C6[MICA/B]_L0, respectively, (lx) SEQ ID Nos: 703 and 707 for 13A9 [MICA/B]_H0_13A9 [MICA/B]_L0, respectively, and (lxi) SEQ ID Nos: 711 and 715 for 1D5 [MICA/B]_H0_1D5 [MICA/B]_L0, respectively, as are generally shown in FIGS. 21 and 22.

In particular embodiments, the V_{H}/V_{L} pairs used in Fabs are selected from the group including: (i) SEQ ID Nos: 239 and 243 for D94837_E11_1 [MICA/B]_H0_D94837_1E11_1 [MICA/B]_L0, respectively, (ii) SEQ ID Nos: 247 and 251 for D94837_1E11_1 [MICA/B]_H1_D94837_1E11_1 [MICA/B]_L1, respectively, (iii) SEQ ID Nos: 255 and 259 for D94837_1E11_1 [MICA/B]_H1_D94837_1E11_1 [MICA/B]_L2, respectively, (iv) SEQ ID Nos: 263 and 267 for D94837_E11_1 [MICA/B]_H2_D94837_E11_1 [MICA/B]_L1, respectively, (v) SEQ ID Nos: 271 and 275 for D94837_1E11_1 [MICA/B]_H2_D94837_E11_1 [MICA/B]_L2, respectively, (vi) SEQ ID Nos: 279 and 283 for 2E5 [MICA/B]_H0_2E5 [MICA/B]_L0, respectively, (vii) SEQ ID Nos: 287 and 291 for 2E5 [MICA/B]_H1_2E5 [MICA/B]_L1, respectively, (viii) SEQ ID Nos: 295 and 299 for 2E5 [MICA/B]_H1_2E5 [MICA/B]_L2, respectively, (ix) SEQ ID Nos: 303 and 307 for 2E5 [MICA/B]_H2_2E5 [MICA/B]_L1, respectively, (x) SEQ ID Nos: 311 and 315 for 2E5 [MICA/B]_H2_2E5 [MICA/B]_L2, respectively, (xi) SEQ ID Nos: 319 and 323 for D94852_2E12 [MICA/B]_H0_D94852_2E12 [MICA/B]_L0, respectively, (xii) SEQ ID Nos: 327 and 331 for D94852_2E12 [MICA/B]_H1_D94852_2E12 [MICA/B]_L1, respectively, (xiii) SEQ ID Nos: 335 and 339 for D94852_2E12 [MICA/B]_H1_D94852_2E12 [MICA/B]_L2, respectively, (xiv) SEQ ID Nos: 343 and 347 for D94852_2E12 [MICA/B]_H2_D94852_2E12 [MICA/B]_L1, respectively, (xv) SEQ ID Nos: 351 and 355 for D94852_2E12 [MICA/B]_H2_D94852_2E12 [MICA/B]_L2, respectively, (xvi) SEQ ID Nos: 359 and 363 for D99136_2F7 [MICA/B]_H0_D99136_2F7 [MICA/B]_L0, respectively, (xvii) SEQ ID Nos: 367 and 371 for D99136_2F7 [MICA/B]_H1_D99136_2F7 [MICA/B]_L1, respectively, (xviii) SEQ ID Nos: 375 and 379 for D99136_2F7 [MICA/B]_H1_D99136_2F7 [MICA/B]_L2, respectively, (xix) SEQ ID Nos: 383 and 387 for D99136_2F7 [MICA/B]_H2_D99136_2F7 [MICA/B]_L1, respectively, (xx) SEQ ID Nos: 391 and 395 for D99136_2F7 [MICA/B]_H2_D99136_2F7 [MICA/B]_L2, respectively, (xxi) SEQ ID Nos: 399 and 403 for D103388_1C7 [MICA/B]_H0_D103388_1C7 [MICA/B]_L0, respectively, (xxii) SEQ ID Nos: 407 and 411 for D103388_1C7 [MICA/B]_H1_D103388_1C7 [MICA/B]_L1, respectively, (xxiii) SEQ ID Nos: 415 and 419 for D103388_1C7 [MICA/B]_H1_D103388_1C7 [MICA/B]_L2, respectively, (xxiv) SEQ ID Nos: 423 and 427 for D103388_1C7 [MICA/B]_H2_D103388_1C7 [MICA/B]_L1, respectively, (xxv) SEQ ID Nos: 431 and 435 for D103388_1C7 [MICA/B]_H2_D103388_1C7 [MICA/B]_L2, respectively, (xxvi) SEQ ID Nos: 439 and 443 for D103388_1D7 [MICA/B]_H0_D103388_1D7 [MICA/B]_L0, respectively, (xxvii) SEQ ID Nos: 447 and 451 for D103388_1D7 [MICA/B]_H1_D103388_1D7 [MICA/B]_L1, respectively, (xxviii) SEQ ID Nos: 455 and 459 for D103388_1D7 [MICA/B]_H1_D103388_1D7 [MICA/B]_L2, respectively, (xxix) SEQ ID Nos: 463 and 467 for D103388_1D7 [MICA/B]_H2_D103388_1D7 [MICA/B]_L1, respectively, (xxx) SEQ ID Nos: 471 and 475 for D103388_1D7 [MICA/B]_H2_D103388_1D7 [MICA/B]_L2, respectively, (xxxi) SEQ ID Nos: 479 and 483 for D105317_1A2 [MICA/B]_H0_D105317_1A2 [MICA/B]_L0, respectively, (xxxii) SEQ ID Nos: 487 and 491 for D105317_1A2 [MICA/B]_H1_D105317_1A2 [MICA/B]_L1, respectively, (xxxiii) SEQ ID Nos: 495 and 499 for D105317_1A2 [MICA/B]_H1_D105317_1A2 [MICA/B]_L2, respectively, (xxxiv) SEQ ID Nos: 503 and 507 for D105317_1A2 [MICA/B]_H2_D105317_1A2 [MICA/B]_L1, respectively, (xxxv) SEQ ID Nos: 511 and 515 for D105317_1A2 [MICA/B]_H2_D105317_1A2 [MICA/B]_L2, respectively, (xxxvi) SEQ ID Nos: 519 and 523 for D99136_2C11 [MICA/B]_H0_D99136_2C11 [MICA/B]_L0, respectively, (xxxvii) SEQ ID Nos: 527 and 531 for D99136_2C11 [MICA/B]_H1_D99136_2C11 [MICA/B]_L1, respectively, (xxxviii) SEQ ID Nos: 535 and 539 for D99136_2C11 [MICA/B]_H1_D99136_2C11 [MICA/B]_L2, respectively, (xxxix) SEQ ID Nos: 543 and 547 for D99136_2C11 [MICA/B]_H2_D99136_2C11 [MICA/B]_L1, respectively, (xl) SEQ ID Nos: 551 and 555 for D99136_2C11 [MICA/B]_H2_D99136_2C11 [MICA/B]_L2, respectively, (xli) SEQ ID Nos: 559 and 563 for D103388_1B11-2 [MICA/B]_H0_D103388_1B11-2 [MICA/B]_L0, respectively, (xlii) SEQ ID Nos: 567 and 571 for D105317_1B6 [MICA/B]_H0_D105317_1B6 [MICA/B]_L0, respectively, (xliii) SEQ ID Nos: 575 and 579 for D105317_1C8 [MICA/B]_H0_D105317_1C8 [MICA/B]_L0, respectively, (xliv) SEQ ID Nos: 583 and 587 for D105317_1F6 [MICA/B]_H0_D105317_1F6 [MICA/B]_L0, respectively, (xlv) SEQ ID Nos: 591 and 595 for D105317_1F7 [MICA/B]_H0_D105317_1F7 [MICA/B]_L0, respectively, (xlvi) SEQ ID Nos: 599 and 603 for D94837_1D3 [MICA/B]_H0_D94837_1D3 [MICA/B]_L0, respectively, (xlvii) SEQ ID Nos: 607 and 611 for D94837_1D8 [MICA/B]_H0_D94837_1D8 [MICA/B]_L0, respectively, (xlviii) SEQ ID Nos: 615 and 619 for D94837_1D9 [MICA/B]_H0_D94837_1D9 [MICA/B]_L0, respectively, (xlix) SEQ ID Nos: 623 and 627 for D94837_1E1 [MICA/B]_H0_D94837_1E1 [MICA/B]_L0, respectively, (1) SEQ ID Nos: 631 and 635 for D94852_2D4 [MICA/B]_H0_D94852_2D4 [MICA/B]_L0, respectively, (li) SEQ ID Nos: 639 and 643 for D94852_2G8 [MICA/B]_H0_D94852_2G8 [MICA/B]_L0, respectively, (lii) SEQ ID Nos: 647 and 651 for D88487_2A8 [MICA/B]_H0_D88487_2A8 [MICA/B]_L0, respectively, (liii) SEQ ID Nos: 655 and 659 for D94837_3B12 [MICA/B]_H0_D94837_3B12 [MICA/B]_L0, respectively, (liv) SEQ ID Nos: 663 and 667 for D99122_1H7 [MICA/B]_H0_D99122_1H7 [MICA/B]_L0, respectively, (lv) SEQ ID Nos: 671 and 675 for D99136_2E8 [MICA/B]_H0_D99136_2E8 [MICA/B]_L0, respectively, (lvi) SEQ ID Nos: 647 and 651 for D88487_2A8 [MICA/B]_H0_D88487_2A8 [MICA/B]_L0, respectively, (lvii) SEQ ID Nos: 679 and 683 for 3F9[MICA/B]_H0_3F9[MICA/B]_L0, respectively, (lviii) SEQ ID Nos: 687 and 691 for 6E1[MICA/B]_H0_6E1[MICA/B]_L0, respectively, (lix) SEQ ID Nos: 695 and 699 for 7C6[MICA/B]_H0_7C6[MICA/B]_L0, respectively, (lx) SEQ ID Nos: 703 and 707 for 13A9 [MICA/B]_H0_13A9 [MICA/B]_L0, respectively, and (lxi) SEQ ID Nos: 711 and 715 for 1D5 [MICA/B]_H0_1D5 [MICA/B]_L0, respectively, as are generally shown in FIGS. 21 and 22.

As will be appreciated by those in the art, suitable MICA/B binding domains can comprise a set of 6 CDRs as depicted in the Figures, either as they are underlined or, in the case where a different numbering scheme is used, as described herein and as shown in Table 2, as the CDRs that are identified using other alignments within the V_{H} and V_{L} sequences of those depicted in FIGS. 21 and 22. Suitable ABDs can also include the entire V_{H} and V_{L} sequences as depicted in these sequences and Figures, used as scFvs or as Fabs. In many of the embodiments herein that contain an Fv to MICA/B, it is the scFv monomer that binds MICA/B. However, in many other embodiments herein that contain an Fv to MICA/B, it is the Fab monomer that binds MICA/B.

In addition to the parental CDR sets disclosed in the figures and sequence listing that form an ABD to MICA/B, provided herein are variant MICA/B ABDs having CDRs that include at least one modification of the MICA/B ABD CDRs disclosed herein (see, e.g., FIGS. 21 and 22). In one embodiment, the MICA/B ABD of the subject heterodimeric antibody includes a set of 6 CDRS with 1, 2, 3, 4, 5, 6, 7, 8, 9, or 10 amino acid modifications as compared to the 6 CDRs of a MICA/B binding domain V_{H}/V_{L} pair as described herein, including the figures and sequence listing. In exemplary embodiments, the MICA/B ABD of the subject heterodimeric antibody includes a set of 6 CDRs with 1, 2, 3, 4, 5, 6, 7, 8, 9, or 10 amino acid modifications as compared to the 6 CDRs of one of the following MICA/B binding domain V_{H}/V_{L} pairs: (i) SEQ ID Nos: 239-246 for D94837_E11_1 [MICA/B]_H0_D94837_E11_1 [MICA/B]_L0, respectively, (ii) SEQ ID Nos: 247-254 for D94837_E11_1 [MICA/B]_H1_D94837_1E11_1 [MICA/B]_L1, respectively, (iii) SEQ ID Nos: 255-262 for D94837_1E11_1 [MICA/B]_H1_D94837_E11_1 [MICA/B]_L2, respectively, (iv) SEQ ID Nos: 263-270 for D94837_1E11_1 [MICA/B]_H2_D94837_1E11_1 [MICA/B]_L1, respectively, (v) SEQ ID Nos: 271-278 for D94837_1E11_1 [MICA/B]_H2_D94837_1E11_1 [MICA/B]_L2, respectively, (vi) SEQ ID Nos: 279-286 for 2E5 [MICA/B]_H0_2E5 [MICA/B]_L0, respectively, (vii) SEQ ID Nos: 287-294 for 2E5 [MICA/B]_H1_2E5 [MICA/B]_L1, respectively, (viii) SEQ ID Nos: 295-302 for 2E5 [MICA/B]_H1_2E5 [MICA/B]_L2, respectively, (ix) SEQ ID Nos: 303-310 for 2E5 [MICA/B]_H2_2E5 [MICA/B]_L1, respectively, (x) SEQ ID Nos: 311-318 for 2E5 [MICA/B]_H2_2E5 [MICA/B]_L2, respectively, (xi) SEQ ID Nos: 319-326 for D94852_2E12 [MICA/B]_H0_D94852_2E12 [MICA/B]_L0, respectively, (xii) SEQ ID Nos: 327-334 for D94852_2E12 [MICA/B]_H1_D94852_2E12 [MICA/B]_L1, respectively, (xiii) SEQ ID Nos: 335-342 for D94852_2E12 [MICA/B]_H1_D94852_2E12 [MICA/B]_L2, respectively, (xiv) SEQ ID Nos: 343-350 for D94852_2E12 [MICA/B]_H2_D94852_2E12 [MICA/B]_L1, respectively, (xv) SEQ ID Nos: 351-358 for D94852_2E12 [MICA/B]_H2_D94852_2E12 [MICA/B]_L2, respectively, (xvi) SEQ ID Nos: 359-366 for D99136_2F7 [MICA/B]_H0_D99136_2F7 [MICA/B]_L0, respectively, (xvii) SEQ ID Nos: 367-374 for D99136_2F7 [MICA/B]_H1_D99136_2F7 [MICA/B]_L1, respectively, (xviii) SEQ ID Nos: 375-382 for D99136_2F7 [MICA/B]_H1_D99136_2F7 [MICA/B]_L2, respectively, (xix) SEQ ID Nos: 383-390 for D99136_2F7 [MICA/B]_H2_D99136_2F7 [MICA/B]_L1, respectively, (xx) SEQ ID Nos: 391-398 for D99136_2F7 [MICA/B]_H2_D99136_2F7 [MICA/B]_L2, respectively, (xxi) SEQ ID Nos: 399-406 for D103388_1C7 [MICA/B]_H0_D103388_1C7 [MICA/B]_L0, respectively, (xxii) SEQ ID Nos: 407-414 for D103388_1C7 [MICA/B]_H1_D103388_1C7 [MICA/B]_L1, respectively, (xxiii) SEQ ID Nos: 415-422 for D103388_1C7 [MICA/B]_H1_D103388_1C7 [MICA/B]_L2, respectively, (xxiv) SEQ ID Nos: 423-430 for D103388_1C7 [MICA/B]_H2_D103388_1C7 [MICA/B]_L1, respectively, (xxv) SEQ ID Nos: 431-438 for D103388_1C7 [MICA/B]_H2_D103388_1C7 [MICA/B]_L2, respectively, (xxvi) SEQ ID Nos: 439-446 for D103388_1D7 [MICA/B]_H0_D103388_1D7 [MICA/B]_L0, respectively, (xxvii) SEQ ID Nos: 447-454 for D103388_1D7 [MICA/B]_H1_D103388_1D7 [MICA/B]_L1, respectively, (xxviii) SEQ ID Nos: 455-462 for D103388_1D7 [MICA/B]H1_D103388_1D7 [MICA/B]_L2, respectively, (xxix) SEQ ID Nos: 463-470 for D103388_1D7 [MICA/B]_H2_D103388_1D7 [MICA/B]_L1, respectively, (xxx) SEQ ID Nos: 471-478 for D103388_1D7 [MICA/B]_H2_D103388_1D7 [MICA/B]_L2, respectively, (xxxi) SEQ ID Nos: 479-486 for D105317_1A2 [MICA/B]_H0_D105317_1A2 [MICA/B]_L0, respectively, (xxxii) SEQ ID Nos: 487-494 for D105317_1A2 [MICA/B]_H1_D105317_1A2 [MICA/B]_L1, respectively, (xxxiii) SEQ ID Nos: 495-502 for D105317_1A2 [MICA/B]_H1_D105317_1A2 [MICA/B]_L2, respectively, (xxxiv) SEQ ID Nos: 503-510 for D105317_1A2 [MICA/B]_H2_D105317_1A2 [MICA/B]_L1, respectively, (xxxv) SEQ ID Nos: 511-518 for D105317_1A2 [MICA/B]_H2_D105317_1A2 [MICA/B]_L2, respectively, (xxxvi) SEQ ID Nos: 519-526 for D99136_2C11 [MICA/B]_H0_D99136_2C11 [MICA/B]_L0, respectively, (xxxvii) SEQ ID Nos: 527-534 for D99136_2C11 [MICA/B]_H1_D99136_2C11 [MICA/B]_L1, respectively, (xxxviii) SEQ ID Nos: 535-542 for D99136_2C11 [MICA/B]_H1_D99136_2C11 [MICA/B]_L2, respectively, (xxxix) SEQ ID Nos: 543-550 for D99136_2C11 [MICA/B]_H2_D99136_2C11 [MICA/B]_L1, respectively, (xl) SEQ ID Nos: 551-558 for D99136_2C11 [MICA/B]_H2_D99136_2C11 [MICA/B]_L2, respectively, (xli) SEQ ID Nos: 559-566 for D103388_1B11-2 [MICA/B]_H0_D103388_1B11-2 [MICA/B]_L0, respectively, (xlii) SEQ ID Nos: 567-574 for D105317_1B6 [MICA/B]_H0_D105317_1B6 [MICA/B]_L0, respectively, (xliii) SEQ ID Nos: 575-582 for D105317_1C8 [MICA/B]_H0_D105317_1C8 [MICA/B]_L0, respectively, (xliv) SEQ ID Nos: 583-590 for D105317_1F6 [MICA/B]_H0_D105317_1F6 [MICA/B]_L0, respectively, (xlv) SEQ ID Nos: 591-598 for D105317_1F7 [MICA/B]_H0_D105317_1F7 [MICA/B]_L0, respectively, (xlvi) SEQ ID Nos: 599-606 for D94837_1D3 [MICA/B]_H0_D94837_1D3 [MICA/B]_L0, respectively, (xlvii) SEQ ID Nos: 607-614 for D94837_1D8 [MICA/B]_H0_D94837_1D8 [MICA/B]_L0, respectively, (xlviii) SEQ ID Nos: 615-622 for D94837_1D9 [MICA/B]_H0_D94837_1D9 [MICA/B]_L0, respectively, (xlix) SEQ ID Nos: 623-630 for D94837_1E1 [MICA/B]_H0_D94837_1E1 [MICA/B]_L0, respectively, (1) SEQ ID Nos: 631-638 for D94852_2D4 [MICA/B]_H0_D94852_2D4 [MICA/B]_L0, respectively, (li) SEQ ID Nos: 639-646 for D94852_2G8 [MICA/B]_H0_D94852_2G8 [MICA/B]_L0, respectively, (lii) SEQ ID Nos: 647-654 for D88487_2A8 [MICA/B]_H0_D88487_2A8 [MICA/B]_L0, respectively, (liii) SEQ ID Nos: 655-662 for D94837_3B12 [MICA/B]_H0_D94837_3B12 [MICA/B]_L0, respectively, (liv) SEQ ID Nos: 663-670 for D99122_1H7 [MICA/B]_H0_D99122_1H7 [MICA/B]_L0, respectively, (lv) SEQ ID Nos: 671-678 for D99136_2E8 [MICA/B]_H0_D99136_2E8 [MICA/B]_L0, respectively, (lvi) SEQ ID Nos: 647-654 for D88487_2A8 [MICA/B]_H0_D88487_2A8 [MICA/B]_L0, respectively, (lvii) SEQ ID Nos: 679-686 for 3F9[MICA/B]_H0_3F9[MICA/B]_L0, respectively, (lviii) SEQ ID Nos: 687-694 for 6E1[MICA/B]_H0_6E1[MICA/B]_L0, respectively, (lix) SEQ ID Nos: 695-702 for 7C6[MICA/B]_H0_7C6[MICA/B]_L0, respectively, (lx) SEQ ID Nos: 703-710 for 13A9 [MICA/B]_H0_13A9 [MICA/B]_L0, respectively, or (lxi) SEQ ID Nos: 711-718 for 1D5 [MICA/B]_H0_1D5 [MICA/B]_L0, respectively. In certain embodiments, the MICA/B ABD of the subject antibody is capable of binding to MICA, as measured by at least one of: (i) a Biacore assay, (ii) a surface plasmon resonance (SPR) assay, (iii) a biolayer interferometry (BLI) assay (e.g., an Octet assay), (iv) flow cytometry, or any combination thereof. In particular embodiments, the MICA/B ABD is capable of binding a human MICA antigen (see, e.g., FIG. 1; SEQ ID NOs: 7-12). In particular embodiments, the MICA/B ABD is capable of binding the extracellular domain (ECD) of a human MICA antigen (see, e.g., FIG. 1; SEQ ID NOs: 7-12). In certain embodiments, the MICA/B ABD of the subject antibody is capable of binding to MICB, as measured by at least one of: (i) a Biacore assay, (ii) a surface plasmon resonance (SPR) assay, (iii) a biolayer interferometry (BLI) assay (e.g., an Octet assay), (iv) flow cytometry, or any combination thereof. In particular embodiments, the MICA/B ABD is capable of binding a human MICB antigen (see, e.g., FIG. 2; SEQ ID NO: 13-16). In particular embodiments, the MICA/B ABD is capable of binding the extracellular domain (ECD) of a human MICB antigen (see, e.g., FIG. 2; SEQ ID NO: 13-16).

In another exemplary embodiment, the MICA/B ABD of the subject antibody includes the variable heavy (V_{H}) domain and variable light (V_{L}) domain of any one of the MICA/B binding domain V_{H}/V_{L} pairs described herein, including the figures and sequence listing.

In some embodiments, the subject antibody includes a MICA/B ABD that includes a variable heavy domain and/or a variable light domain that are variants of a MICA/B ABD V_{H} and/or V_{L} domain disclosed herein. In one embodiment, the variant V_{H} domain and/or variant V_{L} domain has from 1, 2, 3, 4, 5, 6, 7, 8, 9, or 10 amino acid changes from a V_{H} and/or V_{L} domain of a MICA/B ABD described herein, including the figures and sequence listing. In exemplary embodiments, the variant V_{H} domain and/or variant V_{L} domain has from 1, 2, 3, 4, 5, 6, 7, 8, 9 or 10 amino acid changes from a V_{H} and/or V_{L} domain of one of the following MICA/B binding domain V_{H}/V_{L} pairs: (i) SEQ ID Nos: 239 and 243 for D94837_1E11_1 [MICA/B]_H0_D94837_1E11_1 [MICA/B]_L0, respectively, (ii) SEQ ID Nos: 247 and 251 for D94837_1E11_1 [MICA/B]_H1_D94837_1E11_1 [MICA/B]_L1, respectively, (iii) SEQ ID Nos: 255 and 259 for D94837_E11_1 [MICA/B]_H1_D94837_E11_1 [MICA/B]_L2, respectively, (iv) SEQ ID Nos: 263 and 267 for D94837_1E11_1 [MICA/B]_H2_D94837_1E11_1 [MICA/B]_L1, respectively, (v) SEQ ID Nos: 271 and 275 for D94837_1E11_1 [MICA/B]_H2_D94837_1E11_1 [MICA/B]_L2, respectively, (vi) SEQ ID Nos: 279 and 283 for 2E5 [MICA/B]_H0_2E5 [MICA/B]_L0, respectively, (vii) SEQ ID Nos: 287 and 291 for 2E5 [MICA/B]_H1_2E5 [MICA/B]_L1, respectively, (viii) SEQ ID Nos: 295 and 299 for 2E5 [MICA/B]_H1_2E5 [MICA/B]_L2, respectively, (ix) SEQ ID Nos: 303 and 307 for 2E5 [MICA/B]_H2_2E5 [MICA/B]_L1, respectively, (x) SEQ ID Nos: 311 and 315 for 2E5 [MICA/B]_H2_2E5 [MICA/B]_L2, respectively, (xi) SEQ ID Nos: 319 and 323 for D94852_2E12 [MICA/B]_H0_D94852_2E12 [MICA/B]_L0, respectively, (xii) SEQ ID Nos: 327 and 331 for D94852_2E12 [MICA/B]_H1_D94852_2E12 [MICA/B]_L1, respectively, (xiii) SEQ ID Nos: 335 and 339 for D94852_2E12 [MICA/B]_H1_D94852_2E12 [MICA/B]_L2, respectively, (xiv) SEQ ID Nos: 343 and 347 for D94852_2E12 [MICA/B]_H2_D94852_2E12 [MICA/B]_L1, respectively, (xv) SEQ ID Nos: 351 and 355 for D94852_2E12 [MICA/B]_H2_D94852_2E12 [MICA/B]_L2, respectively, (xvi) SEQ ID Nos: 359 and 363 for D99136_2F7 [MICA/B]_H0_D99136_2F7 [MICA/B]_L0, respectively, (xvii) SEQ ID Nos: 367 and 371 for D99136_2F7 [MICA/B]_H1_D99136_2F7 [MICA/B]_L1, respectively, (xviii) SEQ ID Nos: 375 and 379 for D99136_2F7 [MICA/B]_H1_D99136_2F7 [MICA/B]_L2, respectively, (xix) SEQ ID Nos: 383 and 387 for D99136_2F7 [MICA/B]_H2_D99136_2F7 [MICA/B]_L1, respectively, (xx) SEQ ID Nos: 391 and 395 for D99136_2F7 [MICA/B]_H2_D99136_2F7 [MICA/B]_L2, respectively, (xxi) SEQ ID Nos: 399 and 403 for D103388_1C7 [MICA/B]_H0_D103388_1C7 [MICA/B]_L0, respectively, (xxii) SEQ ID Nos: 407 and 411 for D103388_1C7 [MICA/B]_H1_D103388_1C7 [MICA/B]_L1, respectively, (xxiii) SEQ ID Nos: 415 and 419 for D103388_1C7 [MICA/B]_H1_D103388_1C7 [MICA/B]_L2, respectively, (xxiv) SEQ ID Nos: 423 and 427 for D103388_1C7 [MICA/B]_H2_D103388_1C7 [MICA/B]_L1, respectively, (xxv) SEQ ID Nos: 431 and 435 for D103388_1C7 [MICA/B]_H2_D103388_1C7 [MICA/B]_L2, respectively, (xxvi) SEQ ID Nos: 439 and 443 for D103388_1D7 [MICA/B]_H0_D103388_1D7 [MICA/B]_L0, respectively, (xxvii) SEQ ID Nos: 447 and 451 for D103388_1D7 [MICA/B]_H1_D103388_1D7 [MICA/B]_L1, respectively, (xxviii) SEQ ID Nos: 455 and 459 for D103388_1D7 [MICA/B]_H1_D103388_1D7 [MICA/B]_L2, respectively, (xxix) SEQ ID Nos: 463 and 467 for D103388_1D7 [MICA/B]_H2_D103388_1D7 [MICA/B]_L1, respectively, (xxx) SEQ ID Nos: 471 and 475 for D103388_1D7 [MICA/B]_H2_D103388_1D7 [MICA/B]_L2, respectively, (xxxi) SEQ ID Nos: 479 and 483 for D105317_1A2 [MICA/B]_H0_D105317_1A2 [MICA/B]_L0, respectively, (xxxii) SEQ ID Nos: 487 and 491 for D105317_1A2 [MICA/B]_H1_D105317_1A2 [MICA/B]_L1, respectively, (xxxiii) SEQ ID Nos: 495 and 499 for D105317_1A2 [MICA/B]_H1_D105317_1A2 [MICA/B]_L2, respectively, (xxxiv) SEQ ID Nos: 503 and 507 for D105317_1A2 [MICA/B]_H2_D105317_1A2 [MICA/B]_L1, respectively, (xxxv) SEQ ID Nos: 511 and 515 for D105317_1A2 [MICA/B]_H2_D105317_1A2 [MICA/B]_L2, respectively, (xxxvi) SEQ ID Nos: 519 and 523 for D99136_2C11 [MICA/B]_H0_D99136_2C11 [MICA/B]_L0, respectively, (xxxvii) SEQ ID Nos: 527 and 531 for D99136_2C11 [MICA/B]_H1_D99136_2C11 [MICA/B]_L1, respectively, (xxxviii) SEQ ID Nos: 535 and 539 for D99136_2C11 [MICA/B]_H1_D99136_2C11 [MICA/B]_L2, respectively, (xxxix) SEQ ID Nos: 543 and 547 for D99136_2C11 [MICA/B]_H2_D99136_2C11 [MICA/B]_L1, respectively, (xl) SEQ ID Nos: 551 and 555 for D99136_2C11 [MICA/B]_H2_D99136_2C11 [MICA/B]_L2, respectively, (xli) SEQ ID Nos: 559 and 563 for D103388_1B11-2 [MICA/B]_H0_D103388_1B11-2 [MICA/B]_L0, respectively, (xlii) SEQ ID Nos: 567 and 571 for D105317_1B6 [MICA/B]_H0_D105317_1B6 [MICA/B]_L0, respectively, (xliii) SEQ ID Nos: 575 and 579 for D105317_1C8 [MICA/B]_H0_D105317_1C8 [MICA/B]_L0, respectively, (xliv) SEQ ID Nos: 583 and 587 for D105317_1F6 [MICA/B]_H0_D105317_1F6 [MICA/B]_L0, respectively, (xlv) SEQ ID Nos: 591 and 595 for D105317_1F7 [MICA/B]_H0_D105317_1F7 [MICA/B]_L0, respectively, (xlvi) SEQ ID Nos: 599 and 603 for D94837_1D3 [MICA/B]_H0_D94837_1D3 [MICA/B]_L0, respectively, (xlvii) SEQ ID Nos: 607 and 611 for D94837_1D8 [MICA/B]_H0_D94837_1D8 [MICA/B]_L0, respectively, (xlviii) SEQ ID Nos: 615 and 619 for D94837_1D9 [MICA/B]_H0_D94837_1D9 [MICA/B]_L0, respectively, (xlix) SEQ ID Nos: 623 and 627 for D94837_1E1 [MICA/B]_H0_D94837_1E1 [MICA/B]_L0, respectively, (1) SEQ ID Nos: 631 and 635 for D94852_2D4 [MICA/B]_H0_D94852_2D4 [MICA/B]_L0, respectively, (li) SEQ ID Nos: 639 and 643 for D94852_2G8 [MICA/B]_H0_D94852_2G8 [MICA/B]_L0, respectively, (lii) SEQ ID Nos: 647 and 651 for D88487_2A8 [MICA/B]_H0_D88487_2A8 [MICA/B]_L0, respectively, (liii) SEQ ID Nos: 655 and 659 for D94837_3B12 [MICA/B]_H0_D94837_3B12 [MICA/B]_L0, respectively, (liv) SEQ ID Nos: 663 and 667 for D99122_1H7 [MICA/B]_H0_D99122_1H7 [MICA/B]_L0, respectively, (lv) SEQ ID Nos: 671 and 675 for D99136_2E8 [MICA/B]_H0_D99136_2E8 [MICA/B]_L0, respectively, (lvi) SEQ ID Nos: 647 and 651 for D88487_2A8 [MICA/B]_H0_D88487_2A8 [MICA/B]_L0, respectively, (lvii) SEQ ID Nos: 679 and 683 for 3F9[MICA/B]_H0_3F9[MICA/B]_L0, respectively, (lviii) SEQ ID Nos: 687 and 691 for 6E1[MICA/B]_H0_6E1[MICA/B]_L0, respectively, (lix) SEQ ID Nos: 695 and 699 for 7C6[MICA/B]_H0_7C6[MICA/B]_L0, respectively, (lx) SEQ ID Nos: 703 and 707 for 13A9 [MICA/B]_H0_13A9 [MICA/B]_L0, respectively, or (lxi) SEQ ID Nos: 711 and 715 for 1D5 [MICA/B]_H0_1D5 [MICA/B]_L0, respectively. In some embodiments, the changes are in a V_{H} domain depicted in FIGS. 21 and 22 (see, e.g., SEQ ID NOs: 239, 247, 255, 263, 271, 279, 287, 295, 303, 311, 319, 327, 335, 343, 351, 359, 367, 375, 383, 391, 399, 407, 415, 423, 431, 439, 447, 455, 463, 471, 479, 487, 495, 503, 511, 519, 527, 535, 543, 551, 559, 567, 575, 583, 591, 599, 607, 615, 623, 631, 639, 647, 655, 663, 671, 679, 687, 695, 703, and 711). In some embodiments, the changes are in a V_{L} domain depicted in FIGS. 21 and 22 (see, e.g., SEQ ID NOs: 243, 251, 259, 267, 275, 283, 291, 299, 307, 315, 323, 331, 339, 347, 355, 363, 371, 379, 387, 395, 403, 411, 419, 427, 435, 443, 451, 459, 467, 475, 483, 491, 499, 507, 515, 523, 531, 539, 547, 555, 563, 571, 579, 587, 595, 603, 611, 619, 627, 635, 643, 651, 659, 667, 675, 683, 691, 699, 707, and 715). In some embodiments, the changes are in a V_{H} and V_{L} domain depicted in FIGS. 21 and 22 (see, e.g., SEQ ID NOs: 239, 243, 247, 251, 255, 259, 263, 267, 271, 275, 279, 283, 287, 291, 295, 299, 303, 307, 311, 315, 319, 323, 327, 331, 335, 339, 343, 347, 351, 355, 359, 363, 367, 371, 375, 379, 383, 387, 391, 395, 399, 403, 407, 411, 415, 419, 423, 427, 431, 435, 439, 443, 447, 451, 455, 459, 463, 467, 471, 475, 479, 483, 487, 491, 495, 499, 503, 507, 511, 515, 519, 523, 527, 531, 535, 539, 543, 547, 551, 555, 559, 563, 567, 571, 575, 579, 583, 587, 591, 595, 599, 603, 607, 611, 615, 619, 623, 627, 631, 635, 639, 643, 647, 651, 655, 659, 663, 667, 671, 675, 679, 683, 687, 691, 695, 699, 703, 707, 711, and 715). In some embodiments, one or more amino acid changes are in the V_{H} and/or V_{L} framework regions (FR1, FR2, FR3, and/or FR4). In some embodiments, one or more amino acid changes are in one or more CDRs. In certain embodiments, the (variant) MICA/B ABD of the subject antibody is capable of binding to MICA, as measured by at least one of: (i) a Biacore assay, (ii) a SPR assay, (iii) a BLI assay (e.g., an Octet assay), (iv) flow cytometry, or any combination thereof. In particular embodiments, the (variant) MICA/B ABD is capable of binding a human MICA antigen (see, e.g., FIG. 1; SEQ ID NOs: 7-12). In particular embodiments, the (variant) MICA/B ABD is capable of binding the ECD of a human MICA antigen (see, e.g., FIG. 1; SEQ ID NOs: 7-12). In certain embodiments, the (variant) MICA/B ABD of the subject antibody is capable of binding to MICB, as measured by at least one of: (i) a Biacore assay, (ii) a SPR assay, (iii) a BLI assay (e.g., an Octet assay), (iv) flow cytometry, or any combination thereof. In particular embodiments, the (variant) MICA/B ABD is capable of binding a human MICB antigen (see, e.g., FIG. 2; SEQ ID NOs: 13-16). In particular embodiments, the (variant) MICA/B ABD is capable of binding the ECD of a human MICB antigen (see, e.g., FIG. 2; SEQ ID NOs: 13-16).

In one embodiment, the variant V_{H} and/or V_{L} domain is at least about 90%, about 95%, about 96%, about 97%, about 98%, or about 99% identical to the V_{H} and/or V_{L} of a MICA/B ABD as described herein, including the figures and sequence listing. In exemplary embodiments, the variant V_{H} and/or V_{L} domain is at least about 90%, about 95%, about 96%, about 97%, about 98%, or about 99% identical to the V_{H} and/or V_{L} of one of the following MICA/B binding domain V_{H}/V_{L} pairs: (i) SEQ ID Nos: 239 and 243 for D94837_1E11_1 [MICA/B]_H0_D94837_1E11_1 [MICA/B]_L0, respectively, (ii) SEQ ID Nos: 247 and 251 for D94837_1E11_1 [MICA/B]_H1_D94837_1E1_1 [MICA/B]_L1, respectively, (iii) SEQ ID Nos: 255 and 259 for D94837_E11_1 [MICA/B]_H1_D94837_1E11_[MICA/B]_L2, respectively, (iv) SEQ ID Nos: 263 and 267 for D94837_1E11_1 [MICA/B]_H2_D94837_1E11_1 [MICA/B]_L1, respectively, (v) SEQ ID Nos: 271 and 275 for D94837_1E11_1 [MICA/B]_H2_D94837_1E11_1 [MICA/B]_L2, respectively, (vi) SEQ ID Nos: 279 and 283 for 2E5 [MICA/B]_H0_2E5 [MICA/B]_L0, respectively, (vii) SEQ ID Nos: 287 and 291 for 2E5 [MICA/B]_H1_2E5 [MICA/B]_L1, respectively, (viii) SEQ ID Nos: 295 and 299 for 2E5 [MICA/B]_H1_2E5 [MICA/B]_L2, respectively, (ix) SEQ ID Nos: 303 and 307 for 2E5 [MICA/B]_H2_2E5 [MICA/B]_L1, respectively, (x) SEQ ID Nos: 311 and 315 for 2E5 [MICA/B]_H2_2E5 [MICA/B]_L2, respectively, (xi) SEQ ID Nos: 319 and 323 for D94852_2E12 [MICA/B]_H0_D94852_2E12 [MICA/B]_L0, respectively, (xii) SEQ ID Nos: 327 and 331 for D94852_2E12 [MICA/B]_H1_D94852_2E12 [MICA/B]_L1, respectively, (xiii) SEQ ID Nos: 335 and 339 for D94852_2E12 [MICA/B]_H1_D94852_2E12 [MICA/B]_L2, respectively, (xiv) SEQ ID Nos: 343 and 347 for D94852_2E12 [MICA/B]_H2_D94852_2E12 [MICA/B]_L1, respectively, (xv) SEQ ID Nos: 351 and 355 for D94852_2E12 [MICA/B]_H2_D94852_2E12 [MICA/B]_L2, respectively, (xvi) SEQ ID Nos: 359 and 363 for D99136_2F7 [MICA/B]_H0_D99136_2F7 [MICA/B]_L0, respectively, (xvii) SEQ ID Nos: 367 and 371 for D99136_2F7 [MICA/B]_H1_D99136_2F7 [MICA/B]_L1, respectively, (xviii) SEQ ID Nos: 375 and 379 for D99136_2F7 [MICA/B]_H1_D99136_2F7 [MICA/B]_L2, respectively, (xix) SEQ ID Nos: 383 and 387 for D99136_2F7 [MICA/B]_H2_D99136_2F7 [MICA/B]_L1, respectively, (xx) SEQ ID Nos: 391 and 395 for D99136_2F7 [MICA/B]_H2_D99136_2F7 [MICA/B]_L2, respectively, (xxi) SEQ ID Nos: 399 and 403 for D103388_1C7 [MICA/B]_H0_D103388_1C7 [MICA/B]_L0, respectively, (xxii) SEQ ID Nos: 407 and 411 for D103388_1C7 [MICA/B]_H1_D103388_1C7 [MICA/B]_L1, respectively, (xxiii) SEQ ID Nos: 415 and 419 for D103388_1C7 [MICA/B]_H1_D103388_1C7 [MICA/B]_L2, respectively, (xxiv) SEQ ID Nos: 423 and 427 for D103388_1C7 [MICA/B]_H2_D103388_1C7 [MICA/B]_L1, respectively, (xxv) SEQ ID Nos: 431 and 435 for D103388_1C7 [MICA/B]_H2_D103388_1C7 [MICA/B]_L2, respectively, (xxvi) SEQ ID Nos: 439 and 443 for D103388_1D7 [MICA/B]_H0_D103388_1D7 [MICA/B]_L0, respectively, (xxvii) SEQ ID Nos: 447 and 451 for D103388_1D7 [MICA/B]_H1_D103388_1D7 [MICA/B]_L1, respectively, (xxviii) SEQ ID Nos: 455 and 459 for D103388_1D7 [MICA/B]_H1_D103388_1D7 [MICA/B]_L2, respectively, (xxix) SEQ ID Nos: 463 and 467 for D103388_1D7 [MICA/B]_H2_D103388_1D7 [MICA/B]_L1, respectively, (xxx) SEQ ID Nos: 471 and 475 for D103388_1D7 [MICA/B]_H2_D103388_1D7 [MICA/B]_L2, respectively, (xxxi) SEQ ID Nos: 479 and 483 for D105317_1A2 [MICA/B]_H0_D105317_1A2 [MICA/B]_L0, respectively, (xxxii) SEQ ID Nos: 487 and 491 for D105317_1A2 [MICA/B]_H1_D105317_1A2 [MICA/B]_L1, respectively, (xxxiii) SEQ ID Nos: 495 and 499 for D105317_1A2 [MICA/B]_H1_D105317_1A2 [MICA/B]_L2, respectively, (xxxiv) SEQ ID Nos: 503 and 507 for D105317_1A2 [MICA/B]_H2_D105317_1A2 [MICA/B]_L1, respectively, (xxxv) SEQ ID Nos: 511 and 515 for D105317_1A2 [MICA/B]_H2_D105317_1A2 [MICA/B]_L2, respectively, (xxxvi) SEQ ID Nos: 519 and 523 for D99136_2C11 [MICA/B]_H0_D99136_2C11 [MICA/B]_L0, respectively, (xxxvii) SEQ ID Nos: 527 and 531 for D99136_2C11 [MICA/B]_H1_D99136_2C11 [MICA/B]_L1, respectively, (xxxviii) SEQ ID Nos: 535 and 539 for D99136_2C11 [MICA/B]_H1_D99136_2C11 [MICA/B]_L2, respectively, (xxxix) SEQ ID Nos: 543 and 547 for D99136_2C11 [MICA/B]_H2_D99136_2C11 [MICA/B]_L1, respectively, (xl) SEQ ID Nos: 551 and 555 for D99136_2C11 [MICA/B]_H2_D99136_2C11 [MICA/B]_L2, respectively, (xli) SEQ ID Nos: 559 and 563 for D103388_1B11-2 [MICA/B]_H0_D103388_1B11-2 [MICA/B]_L0, respectively, (xlii) SEQ ID Nos: 567 and 571 for D105317_1B6 [MICA/B]_H0_D105317_1B6 [MICA/B]_L0, respectively, (xliii) SEQ ID Nos: 575 and 579 for D105317_1C8 [MICA/B]_H0_D105317_1C8 [MICA/B]_L0, respectively, (xliv) SEQ ID Nos: 583 and 587 for D105317_1F6 [MICA/B]_H0_D105317_1F6 [MICA/B]_L0, respectively, (xlv) SEQ ID Nos: 591 and 595 for D105317_1F7 [MICA/B]_H0_D105317_1F7 [MICA/B]_L0, respectively, (xlvi) SEQ ID Nos: 599 and 603 for D94837_1D3 [MICA/B]_H0_D94837_1D3 [MICA/B]_L0, respectively, (xlvii) SEQ ID Nos: 607 and 611 for D94837_1D8 [MICA/B]_H0_D94837_1D8 [MICA/B]_L0, respectively, (xlviii) SEQ ID Nos: 615 and 619 for D94837_1D9 [MICA/B]_H0_D94837_1D9 [MICA/B]_L0, respectively, (xlix) SEQ ID Nos: 623 and 627 for D94837_1E1 [MICA/B]_H0_D94837_1E1 [MICA/B]_L0, respectively, (1) SEQ ID Nos: 631 and 635 for D94852_2D4 [MICA/B]_H0_D94852_2D4 [MICA/B]_L0, respectively, (li) SEQ ID Nos: 639 and 643 for D94852_2G8 [MICA/B]_H0_D94852_2G8 [MICA/B]_L0, respectively, (lii) SEQ ID Nos: 647 and 651 for D88487_2A8 [MICA/B]_H0_D88487_2A8 [MICA/B]_L0, respectively, (liii) SEQ ID Nos: 655 and 659 for D94837_3B12 [MICA/B]_H0_D94837_3B12 [MICA/B]_L0, respectively, (liv) SEQ ID Nos: 663 and 667 for D99122_1H7 [MICA/B]_H0_D99122_1H7 [MICA/B]_L0, respectively, (lv) SEQ ID Nos: 671 and 675 for D99136_2E8 [MICA/B]_H0_D99136_2E8 [MICA/B]_L0, respectively, (lvi) SEQ ID Nos: 647 and 651 for D88487_2A8 [MICA/B]_H0_D88487_2A8 [MICA/B]_L0, respectively, (lvii) SEQ ID Nos: 679 and 683 for 3F9[MICA/B]_H0_3F9[MICA/B]_L0, respectively, (lviii) SEQ ID Nos: 687 and 691 for 6E1[MICA/B]_H0_6E1[MICA/B]_L0, respectively, (lix) SEQ ID Nos: 695 and 699 for 7C6[MICA/B]_H0_7C6[MICA/B]_L0, respectively, (lx) SEQ ID Nos: 703 and 707 for 13A9 [MICA/B]_H0_13A9 [MICA/B]_L0, respectively, or (lxi) SEQ ID Nos: 711 and 715 for 1D5 [MICA/B]_H0_1D5 [MICA/B]_L0, respectively. In some embodiments, the (variant) MICA/B ABD includes a V_{H} that is at least about 90%, about 95%, about 96%, about 97%, about 98%, or about 99% identical to a V_{H} domain depicted in FIGS. 21 and 22 (see, e.g., SEQ ID NOs: 239, 247, 255, 263, 271, 279, 287, 295, 303, 311, 319, 327, 335, 343, 351, 359, 367, 375, 383, 391, 399, 407, 415, 423, 431, 439, 447, 455, 463, 471, 479, 487, 495, 503, 511, 519, 527, 535, 543, 551, 559, 567, 575, 583, 591, 599, 607, 615, 623, 631, 639, 647, 655, 663, 671, 679, 687, 695, 703, and 711). In some embodiments, the (variant) MICA/B ABD includes a V_{L} that is at least about 90%, about 95%, about 96%, about 97%, about 98%, or about 99% identical to a V_{L} domain depicted in FIGS. 21 and 22 (see, e.g., SEQ ID NOs: 243, 251, 259, 267, 275, 283, 291, 299, 307, 315, 323, 331, 339, 347, 355, 363, 371, 379, 387, 395, 403, 411, 419, 427, 435, 443, 451, 459, 467, 475, 483, 491, 499, 507, 515, 523, 531, 539, 547, 555, 563, 571, 579, 587, 595, 603, 611, 619, 627, 635, 643, 651, 659, 667, 675, 683, 691, 699, 707, and 715). In some embodiments, the (variant) MICA/B ABD include a V_{H} and/or a V_{L} that is at least about 90%, about 95%, about 96%, about 97%, about 98%, or about 99% identical to a V_{H} domain and/or a V_{L} domain depicted in FIGS. 21 and 22 (see, e.g., SEQ ID NOs: 239, 243, 247, 251, 255, 259, 263, 267, 271, 275, 279, 283, 287, 291, 295, 299, 303, 307, 311, 315, 319, 323, 327, 331, 335, 339, 343, 347, 351, 355, 359, 363, 367, 371, 375, 379, 383, 387, 391, 395, 399, 403, 407, 411, 415, 419, 423, 427, 431, 435, 439, 443, 447, 451, 455, 459, 463, 467, 471, 475, 479, 483, 487, 491, 495, 499, 503, 507, 511, 515, 519, 523, 527, 531, 535, 539, 543, 547, 551, 555, 559, 563, 567, 571, 575, 579, 583, 587, 591, 595, 599, 603, 607, 611, 615, 619, 623, 627, 631, 635, 639, 643, 647, 651, 655, 659, 663, 667, 671, 675, 679, 683, 687, 691, 695, 699, 703, 707, 711, and 715). In certain embodiments, the (variant) MICA/B ABD of the subject antibody is capable of binding to MICA as measured by at least one of: (i) a Biacore assay, (ii) a SPR assay, (iii) a BLI assay (e.g., an Octet assay), (iv) flow cytometry, or any combination thereof. In particular embodiments, the (variant) MICA/B ABD is capable of binding a human MICA antigen (see, e.g., FIG. 1; SEQ ID NOs: 7-12). In particular embodiments, the (variant) MICA/B ABD is capable of binding the ECD of a human MICA antigen (see, e.g., FIG. 1; SEQ ID NOs: 7-12). In certain embodiments, the (variant) MICA/B ABD of the subject antibody is capable of binding to MICB as measured by at least one of: (i) a Biacore assay, (ii) a SPR assay, (iii) a BLI assay (e.g., an Octet assay), (iv) flow cytometry, or any combination thereof. In particular embodiments, the (variant) MICA/B ABD is capable of binding a human MICB antigen (see, e.g., FIG. 2; SEQ ID NOs: 13-16). In particular embodiments, the (variant) MICA/B ABD is capable of binding the ECD of a human MICB antigen (see, e.g., FIG. 2; SEQ ID NOs: 13-16).

### B. NKp46 Antigen Binding Domains

Herein is provided monoclonal and bispecific antibodies (e.g., the anti-NKp46×anti-MICA/B antibodies provided herein), and fusion proteins that contain ABDs that bind to NKp46. Suitable sets of 6 CDRs (vhCDR1-3 and vlCDR1-3; see, e.g., SEQ ID NOs: 720-722 and 724-726 and 728-730, and 732-734, respectively) and/or V_{H} and V_{L} domains (see, e.g., SEQ ID NOs: 719 and 723 and 727 and 731, respectively) are depicted in FIGS. 23 and 24. In some embodiments, the heterodimeric antibody is a 1+1 Fab×scFv, 1+1 empty×Fab-scFv, 2+1 Fab×Fab-scFv, 2+1 Fab2×scFv, or 2+1 mAb-scFv format antibody (see, e.g., FIGS. 25 and 31-35).

In some embodiments, the NKp46 ABD has a set of vhCDRs selected from the vhCDR1, vhCDR2, and vhCDR3 sequences from a V_{H} selected from the group including: (i) 2C10A3.372[NKp46]_H1 (SEQ ID NO: 720-722), and (ii) NKp46-A[NKp46]_H (SEQ ID NO: 728-730), as shown in FIGS. 23 and 24. In some embodiments, the vhCDR1, vhCDR2, and vhCDR3 sequences of the NKp46 ABD are SEQ ID NOs: 720-722 (such as, for example, in 2C10A3.372[NKp46]_H1). In other embodiments, the vhCDR1, vhCDR2, and vhCDR3 sequences of the NKp46 ABD are SEQ ID NOs: 728-730 (such as, for example, in NKp46-A[NKp46]_H).

In some embodiments, the V_{H} domain of the NKp46 ABD is selected from the group including: (i) 2C10A3.372[NKp46]_H1 (SEQ ID NO: 719), and (ii) NKp46-A[NKp46]_H (SEQ ID NO: 727), as shown in FIGS. 23 and 24.

In some embodiments, the NKp46 ABD has a set of vlCDRs selected from the vlCDR1, vlCDR2, and vlCDR3 sequences from a V_{L} selected from the group including: (i) 2C10A3.372[NKp46]_L1 (SEQ ID NO: 724-726), and (ii) NKp46-A[NKp46]_L (SEQ ID NO: 732-734), as shown in FIGS. 23 and 24. In some embodiments, the vlCDR1, vlCDR2, and vlCDR3 sequences of the NKp46 ABD are SEQ ID NOs: 724-726 (such as, for example, in 2C10A3.372[NKp46]_L1). In other embodiments, the vlCDR1, vlCDR2, and vlCDR3 sequences of the NKp46 ABD are SEQ ID NOs: 732-734 (such as, for example, in NKp46-A[NKp46]_L).

In some embodiments, the V_{L} domain of the NKp46 ABD is selected from the group including: (i) 2C10A3.372[NKp46]_L1 (SEQ ID NO: 723), and (ii) NKp46-A[NKp46]_L (SEQ ID NO: 731), as shown in FIGS. 23 and 24.

Accordingly, included herein are NKp46 ABDs that have a set of 6 CDRs (i.e., vhCDR1, vhCDR2, vhCDR3, vlCDR1, vlCDR2, and vlCDR3) from V_{H}/V_{L} pairs selected from the group including: (i) SEQ ID Nos: 720-722 for vhCDR1-3 and SEQ ID NOs: 724-726 for vlCDR1-3 of 2C10A3.372[NKp46]_H1_2C10A3.372[NKp46]_L1, respectively, and (ii) SEQ ID Nos: 728-730 for vhCDR1-3 and SEQ ID NOs: 732-734 for vlCDR1-3 of NKp46-A[NKp46]_H_NKp46-A[NKp46]_L, respectively, as are generally shown in FIGS. 23 and 24.

Additionally, included herein are NKp46 ABDs that have V_{H}/V_{L} pairs selected from the group including: (i) SEQ ID NOs: 719 and 723 for 2C10A3.372[NKp46]_H1_2C10A3.372[NKp46]_L1, respectively, and (ii) SEQ ID NOs: 727 and 731 for NKp46-A[NKp46]_H_NKp46-A[NKp46]_L, respectively, as are generally shown in FIGS. 23 and 24.

In particular embodiments, the V_{H}/V_{L} pairs used in scFvs are selected from the group including: (i) SEQ ID NOs: 719 and 723 for 2C10A3.372[NKp46]_H1_2C10A3.372[NKp46]_L1, respectively, and (ii) SEQ ID NOs: 727 and 731 for NKp46-A[NKp46]_H_NKp46-A[NKp46]_L, respectively, as are generally shown in FIGS. 23 and 24.

In particular embodiments, the V_{H}/V_{L} pairs used in Fabs are selected from the group including: (i) SEQ ID NOs: 719 and 723 for 2C10A3.372[NKp46]_H1_2C10A3.372[NKp46]_L1, respectively, and (ii) SEQ ID NOs: 727 and 731 for NKp46-A[NKp46]_H_NKp46-A[NKp46]_L, respectively, as are generally shown in FIGS. 23 and 24.

As will be appreciated by those in the art, suitable NKp46 binding domains can comprise a set of 6 CDRs as depicted in the Figures, either as they are underlined or, in the case where a different numbering scheme is used, as described herein and as shown in Table 2, as the CDRs that are identified using other alignments within the V_{H} and V_{L} sequences of those depicted in FIGS. 23 and 24. Suitable ABDs can also include the entire V_{H} and V_{L} sequences as depicted in these sequences and Figures, used as scFvs or as Fabs. In many of the embodiments herein that contain an Fv to NKp46, it is the scFv monomer that binds NKp46. However, in many other embodiments herein that contain an Fv to NKp46, it is the Fab monomer that binds NKp46.

In addition to the parental CDR sets disclosed in the figures and sequence listing that form an ABD to NKp46, provided herein are variant NKp46 ABDs having CDRs that include at least one modification of the NKp46 ABD CDRs disclosed herein (see, e.g., FIGS. 23 and 24). In one embodiment, the NKp46 ABD of the subject heterodimeric antibody includes a set of 6 CDRS with 1, 2, 3, 4, 5, 6, 7, 8, 9, or 10 amino acid modifications as compared to the 6 CDRs of a NKp46 binding domain V_{H}/V_{L} pair as described herein, including the figures and sequence listing. In exemplary embodiments, the NKp46 ABD of the subject heterodimeric antibody includes a set of 6 CDRs with 1, 2, 3, 4, 5, 6, 7, 8, 9, or 10 amino acid modifications as compared to the 6 CDRs of one of the following NKp46 binding domain V_{H}/V_{L} pairs: (i) SEQ ID NOs: 719 and 723 for 2C10A3.372[NKp46]_H1_2C10A3.372[NKp46]_L1, respectively, or (ii) SEQ ID NOs: 727 and 731 for NKp46-A[NKp46]_H_NKp46-A[NKp46]_L, respectively. In certain embodiments, the (variant) NKp46 ABD of the subject antibody is capable of binding to NKp46, as measured by at least one of: (i) a Biacore assay, (ii) a surface plasmon resonance (SPR) assay, (iii) a biolayer interferometry (BLI) assay (e.g., an Octet assay), (iv) flow cytometry, or any combination thereof. In particular embodiments, the (variant) NKp46 ABD is capable of binding a human NKp46 antigen (see, e.g., FIG. 3; SEQ ID NO: 17). In particular embodiments, the (variant) NKp46 ABD is capable of binding the extracellular domain (ECD) of a human NKp46 antigen (see, e.g., FIG. 3; SEQ ID NO: 17).

In another exemplary embodiment, the NKp46 ABD of the subject antibody includes the variable heavy (V_{H}) domain and variable light (V_{L}) domain of any one of the NKp46 binding domain V_{H}/V_{L} pairs described herein, including the figures and sequence listing

In some embodiments, the subject antibody includes a NKp46 ABD that includes a variable heavy domain and/or a variable light domain that are variants of a NKp46 ABD V_{H} and/or V_{L} domain disclosed herein. In one embodiment, the variant V_{H} domain and/or variant V_{L} domain has from 1, 2, 3, 4, 5, 6, 7, 8, 9, or 10 amino acid changes from a V_{H} and/or V_{L} domain of a NKp46 ABD described herein, including the figures and sequence listing. In exemplary embodiments, the variant V_{H} domain and/or variant V_{L} domain has from 1, 2, 3, 4, 5, 6, 7, 8, 9 or 10 amino acid changes from a V_{H} and/or V_{L} domain of one of the following NKp46 binding domain V_{H}/V_{L} pairs: (i) SEQ ID NOs: 719 and 723 for 2C10A3.372[NKp46]_H1_2C10A3.372[NKp46]_L1, respectively, or (ii) SEQ ID NOs: 727 and 731 for NKp46-A[NKp46]_H_NKp46-A[NKp46]_L, respectively. In some embodiments, the changes are in a V_{H} domain depicted in FIGS. 23 and 24 (see, e.g., SEQ ID NOs: 719 and 727). In some embodiments, the changes are in a V_{L} domain depicted in FIGS. 23 and 24 (see, e.g., SEQ ID NOs: 723 and 731). In some embodiments, the changes are in a V_{H} and V_{L} domain depicted in FIGS. 23 and 24 (see, e.g., SEQ ID NOs: 719, 723, 727 and 731). In some embodiments, one or more amino acid changes are in the V_{H} and/or V_{L} framework regions (FR1, FR2, FR3, and/or FR4). In some embodiments, one or more amino acid changes are in one or more CDRs. In certain embodiments, the (variant) NKp46 ABD of the subject antibody is capable of binding to NKp46, as measured by at least one of: (i) a Biacore assay, (ii) a surface plasmon resonance (SPR) assay, (iii) a biolayer interferometry (BLI) assay (e.g., an Octet assay), (iv) flow cytometry, or any combination thereof. In particular embodiments, the (variant) NKp46 ABD is capable of binding a human NKp46 antigen (see, e.g., FIG. 3; SEQ ID NO: 17). In particular embodiments, the (variant) NKp46 ABD is capable of binding the extracellular domain (ECD) of a human NKp46 antigen (see, e.g., FIG. 3; SEQ ID NO: 17).

In one embodiment, the variant V_{H} and/or V_{L} domain is at least about 90%, about 95%, about 96%, about 97%, about 98%, or about 99% identical to the V_{H} and/or V_{L} of a NKp46 ABD as described herein, including the figures and sequence listing. In exemplary embodiments, the variant V_{H} and/or V_{L} domain is at least about 90%, about 95%, about 96%, about 97%, about 98%, or about 99% identical to the V_{H} and/or V_{L} of one of the following NKp46 binding domain V_{H}/V_{L} pairs: (i) SEQ ID NOs: 719 and 723 for 2C10A3.372[NKp46]_H1_2C10A3.372[NKp46]_L1, respectively, or (ii) SEQ ID NOs: 727 and 731 for NKp46-A[NKp46]_H_NKp46-A[NKp46]_L, respectively. In some embodiments, the (variant) NKp46 ABD includes a V_{H} that is at least about 90%, about 95%, about 96%, about 97%, about 98%, or about 99% identical to a V_{H} domain depicted in FIGS. 23 and 24 (see, e.g., SEQ ID NOs: 719 and 727). In some embodiments, the (variant) NKp46 ABD includes a V_{L} that is at least about 90%, about 95%, about 96%, about 97%, about 98%, or about 99% identical to a V_{L} domain depicted in FIGS. 23 and 24 (see, e.g., SEQ ID NOs: 723 and 731). In some embodiments, the (variant) NKp46 ABD include a V_{H} and/or a V_{L} that is at least about 90%, about 95%, about 96%, about 97%, about 98%, or about 99% identical to a V_{H} domain and/or a V_{L} domain depicted in FIGS. 23 and 24 (see, e.g., SEQ ID NOs: 719, 723, 727 and 731). In certain embodiments, the (variant) NKp46 ABD of the subject antibody is capable of binding to NKp46, as measured by at least one of: (i) a Biacore assay, (ii) a surface plasmon resonance (SPR) assay, (iii) a biolayer interferometry (BLI) assay (e.g., an Octet assay), (iv) flow cytometry, or any combination thereof. In particular embodiments, the (variant) NKp46 ABD is capable of binding a human NKp46 antigen (see, e.g., FIG. 3; SEQ ID NO: 17). In particular embodiments, the (variant) NKp46 ABD is capable of binding the extracellular domain (ECD) of a human NKp46 antigen (see, e.g., FIG. 3; SEQ ID NO: 17).

### C. Linkers

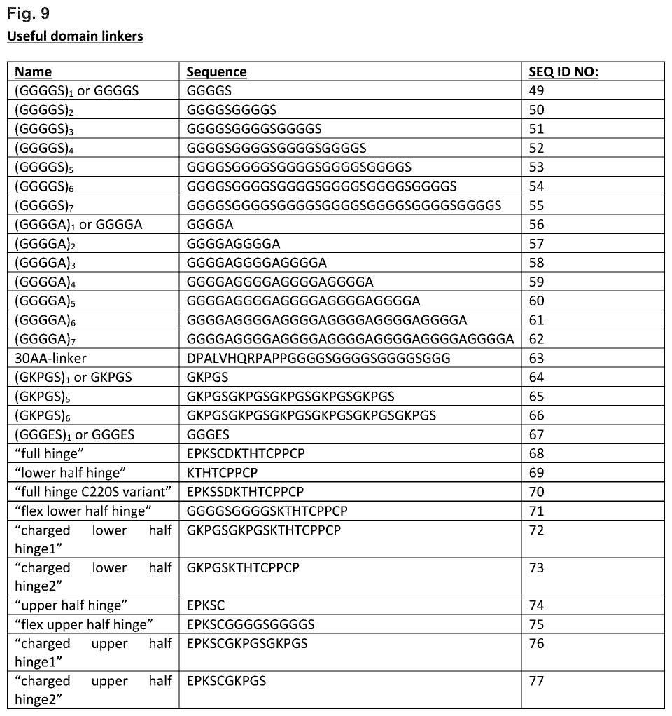

As shown herein, there are a number of suitable linkers (for use as either domain linkers or scFv linkers) that can be used to covalently attach the recited domains (e.g., scFvs, Fabs, Fe domains, etc.), including traditional peptide bonds, generated by recombinant techniques. Exemplary linkers to attach domains of the subject antibody to each other are depicted in FIG. 9. In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n (SEQ ID NO: 3), (GGGGS)n (SEQ ID NO: 4), and (GGGS)n (SEQ ID NO: 5), where n is an integer of at least one (and generally from 3 to 4), glycine-alanine polymers, alanine-serine polymers, and other flexible linkers, some of which are shown in FIG. 9. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of C_{L}/CH1 domain but not all residues of C_{L}CH1 domain; for example, the first 5-12 amino acid residues of the C_{L}/CH1 domains. Linkers can be derived from immunoglobulin light chain, for example C_{κ} or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins.

In some embodiments, the linker is a “domain linker,” used to link any two domains as outlined herein together. For example, in FIG. 9 there may be a domain linker that attaches the C-terminus of a CH1 domain of a Fab to the N-terminus of a scFv, with another optional domain linker attaching the C-terminus of the scFv to a CH2 domain (although in many embodiments the hinge is used as this domain linker). While any suitable linker can be used, many embodiments utilize a glycine-serine polymer as the domain linker, including for example (GS)n, (GSGGS)n (SEQ ID NO: 3), (GGGGS)n (SEQ ID NO: 4), and (GGGS)n (SEQ ID NO: 5), where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness,” as outlined below, charged domain linkers, as used in some embodiments of scFv linkers can be used. Exemplary useful domain linkers are depicted in FIG. 9.

With particular reference to the domain linker used to attach the scFv domain to the Fc domain in the “2+1” format, there are several domain linkers that find particular use, including “full hinge C220S variant,” “flex half hinge,” “charged half hinge 1,” and “charged half hinge 2” as shown in FIG. 9.

In some embodiments, the linker is a “scFv linker,” used to covalently attach the V_{H} and V_{L} domains as discussed herein. In many cases, the scFv linker is a charged scFv linker, a number of which are shown in FIG. 8. Accordingly, in some embodiments, the antibodies described herein further provide charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen.

Charged domain linkers can also be used to increase the pI separation of the monomers of the antibodies described herein as well, and thus those included in FIG. 9 can be used in any embodiment herein where a linker is utilized.

### V. Antibodies

The antibodies provided herein include different antibody domains as is more fully described above. As described herein and known in the art, the antibodies include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains. It should be noted that the term “Fc domain” includes both the CH2-CH3 (and optionally the hinge, hinge-CH2-CH3) of a single monomer, as well as the dimer of two Fc domains that self-assemble. That is, the heavy chain of an antibody has an Fc domain that is a single polypeptide, while the assembled bispecific antibody has an Fc domain that contains two polypeptides. Various antibody domains included in the bispecific, heterodimeric antibodies are more fully described below.

In particular, the formats depicted in FIGS. 25A-25E are usually referred to as “heterodimeric antibodies,” meaning that the protein has at least two associated Fc sequences self-assembled into a heterodimeric Fc domain and at least two Fv regions, whether as Fabs or as scFvs.

Described below are useful variant Fc domains that include amino acid modifications (i.e., substitutions, insertions, or deletions) to enhance FcγR-mediated cytotoxicity, increase serum half-life, and facilitate the self-assembly and/or purification of the heterodimeric antibodies provided. Also, exemplary anti-NKp46×anti-MICA/B bispecific antibodies that include such variant Fc domains are described below and set forth in the Figures and the corresponding sequences.

### A. Chimeric and Humanized Antibodies

In certain embodiments, the antibodies described herein comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants described herein). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants described herein). In some embodiments, the amino acid differences are in one or more of the 6 CDRs. In some embodiments, the amino acid differences are in a V_{H} and/or V_{L} framework region.

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### B. Fc Variants for Increasing Antibody-Dependent Cellular Cytotoxicity (ADCC)

There are a number of useful Fc substitutions that can be made to alter binding to one or more of the FcγR receptors. Substitutions that result in increased binding (or in some cases, decreased binding) can be useful. For example, it is known that increased binding to FcγRIIIa can result in increased ADCC (antibody dependent cell-mediated cytotoxicity). In some instances, decreased binding to FcγRIIb (an inhibitory receptor) can be beneficial as well. Amino acid substitutions that find use in the present invention include those listed in U.S. Ser. No. 11/124,620 (particularly FIG. 41), U.S. Ser. Nos. 11/174,287, 11/396,495, 11/538,406, all of which are expressly incorporated herein by reference in their entirety and specifically for the variants disclosed therein.

In some embodiments, provided herein are bispecific antibodies containing Fc variants that increase antibody-dependent cellular cytotoxicity (ADCC; the cell-mediated reaction wherein nonspecific cytotoxic cells that express FcγRs recognize bound antibody on a target cell and subsequently cause lysis of the target cell) activity of the antibodies. In other words, the heterodimeric antibodies encompassed by the disclosure herein include amino acid substitutions in each or both of the Fc monomeric domains of a parental sequence, usually IgG1, that can enhance ADCC.

In some embodiments, the Fc ADCC variants (e.g., ADCC-enhanced Fc variants) comprise amino acid substitution(s) selected from the group including: E333A, K334A, S239D, S239E, I332D, I332E, S239D/I332E, S239E/I332E, S239D/I332D, S239E/I332D, S239D/A330L/I332E, S239D/A330L, A330L/I332E, F243L, F243L/R292P/Y300L/V305I/P396L, I332E/P247I/A339Q, S298A/E333A, S298A/E333A/K334A, V264I/I332E, S298A, S298A/I332E, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/1332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E298R, S324G, E272R, P227G, G236S, D221K, H224E, K246H, D249Y, R255Y, E258H, T260H, G281D, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering. In some embodiments, the amino acid substitution(s) present in an Fc ADCC variants are selected from the group including: E333A, K334A, S239D, S239E, I332D, I332E, S239D/I332E, S239E/I332E, S239D/I332D, S239E/I332D, S239D/A330L/I332E, S239D/A330L, A330L/I332E, S239D/I332N, S239N/I332D, A330Y/I332E, A330L/I332E, L328V/I332E, L328T/I332E, S239Q/V264I/I332E, S239E/V264I/A330Y/I332E, L235D/S239D/A330Y/I332E, S239D/V240I/A330Y/I332E, S239D/V264T/A330Y/I332E, S239D/K326E/A330Y/I32E, S234D/K326T/A330Y/I332E, E274R, P227G, G236S, D221K, H224E, K246H, D249Y, R255Y, E258H, E258Y, T260H, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering.

In some embodiments, a first Fc domain and/or a second Fc domain of the bispecific antibody provided comprise an Fc ADCC variant selected from the group including: E333A, K334A, S298A/E333A, S298A/E333A/K334A, S239D, S239E, I332D, I332E, S239D/I332E, S239E/I332E, S239D/I332D, S239E/I332D, S239D/A330L/I332E, S239D/A330L, A330L/1332E, S239D/I332N, S239N/I332D, A330Y/I332E, A330L/I332E, L328V/I332E, L328T/I332E, S239Q/V264I/I332E, S239E/V264I/A330Y/I332E, L235D/S239D/A330Y/I332E, S239D/V240I/A330Y/I332E, S239D/V264T/A330Y/I332E, S239D/K326E/A330Y/I32E, S234D/K326T/A330Y/I332E, E274R, P227G, G236S, D221K, H224E, K246H, D249Y, R255Y, E258H, E258Y, T260H, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering.

In some embodiments, one or more of these variants can be included either in both of the Fc monomeric domains or in only one of the Fc monomeric domains of a heterodimeric antibody. In some embodiments, an anti-NKp46×anti-MICA/B bispecific antibody described includes ADCC-enhanced variants which includes one or more amino acid modifications in a first Fc domain and/or a second Fc domain, in other words, in the Fc domain of a first monomer, in the Fc domain of a second monomer, or in the Fc domains of both monomers. In some instances, a first Fc domain includes an Fc ADCC variant, and a second Fc domain does not include an Fc ADCC variant, resulting in an asymmetrical distribution of Fc ADCC variants. In other instances, a first Fc domain includes an Fc ADCC variant, and a second Fc domain includes an Fc ADCC variant. In one embodiment, the Fc ADCC variant of the first and second Fc domains can be the same amino acid substitution. Also, in one embodiment, the Fc ADCC variant of the first and second Fc domains can be different amino acid substitution.

In some embodiments, the Fc ADCC variants described bind with greater affinity to the FcγRIIIa (CD16A) human receptor. In some embodiments, the Fc variants have affinity for FcγRIIIa (CD16A) that is at least 1-fold, 5-fold, 10-fold, 100-fold, 200-fold, or 300-fold greater than that of the parental Fc domain.

In some embodiments, the Fc ADCC variants described can mediate effector function more effectively in the presence of effector cells. In some embodiments, the Fc variants mediate ADCC that is greater than that mediated by the parental Fc domain. In certain embodiments, the Fc variants mediate ADCC that is at least 5-fold, 10-fold, 20-fold, 30-fold, 40-fold, or 50-fold greater than that mediated by the parental Fc domain.

Additional detailed descriptions of Fc variants that may enhance ADCC are provided in WO2004/029207, which is expressly incorporated herein by reference in its entirety and specifically for the variants disclosed therein.

### 1. Fc v90 Variants

In some embodiments, an Fc domain with enhanced binding to human FcγRIIIa (CD16A) and thus increased ADCC activity (“an Fc ADCC variant”) utilizes the amino acid substitutions S239D/I332E (sometimes referred to as the “v90 variants”) in the CH2 domain of one or both of the monomeric Fc domains, according to EU numbering. In some embodiments, a bispecific antibody described herein comprises the Fc v90 variants (e.g., amino acid substitutions S239D/I332E) in both Fc domains. In some embodiments, a bispecific antibody described herein comprises the Fc v90 variants in only one of the monomeric Fc domains. In some embodiments, the antibody comprises the Fc v90 variants in one of the monomeric Fc domains and lacks the Fc v90 variants in another Fc domain. In some embodiments, the antibody comprises the Fc v90 variants in an Fc domain and an amino acid substitution S239D in the CH2 domain of another Fc domain, according to EU numbering. In certain embodiments, the antibody comprises the Fc v90 variants in an Fc domain and an amino acid substitution I332E in the CH2 domain of another Fc domain, according to EU numbering. In some embodiments, the antibody comprises the Fc v90 variants in an Fc domain and lacks an amino acid substitution selected from S239D, I332E and S239D/I332E in the CH2 domain of another Fc domain, according to EU numbering. In some embodiments, one monomeric Fc domain comprises the S239D variant and the other comprises the 1332E variant. In some embodiments, one monomeric Fc domain comprises the S239D variant and the other comprises no Fc ADCC variant. In some embodiments, one monomeric Fc domain comprises the 1332E variant and the other comprises no Fc ADCC variant.

As will be appreciated by those in the art, in the case of these asymmetrical Fc ADCC variants, which monomer receives which variant(s) can be based on the “strandedness” outlined herein; that is, it may be useful to calculate the pI of different combinations and utilize the Fc ADCC variants such that the pIs of the two monomers are different to facilitate purification.

In some embodiments, monomer 1 comprises a first Fc v90 variants, and monomer 2 comprises the amino acid substitution S239D or 1332E. In some embodiments, monomer 1 comprises the Fc V90 variants, and monomer 2 does not comprise the amino acid substitution(s) S239D, 1332E or S239D/I332E. In some embodiments, at least one of the Fc domains of the bispecific antibody comprises the Fc v90 variants. A first Fc domain may comprise the Fc v90 variants, or it may comprise a parental sequence relative to the Fc v90 variants (e.g., a wild-type Fc domain, a Fc domain with one or more amino acid modifications that improves ADCC but does not include S239D, I332E or S239D/I332E substitutions, and the like). In such instances where at least one of the Fc domains comprises a parental sequence, relative to the Fc v90 variants, for the purposes of this section, this Fc domain may be referred to as a “WT Fc domain” with respect to the S239 and 1332 positions of the Fc domain. In some embodiments, the antibody described herein comprises an Fc domain having an amino acid substitution of either S239D, I332E, or S239D/I332E, and another Fc domain having an amino acid substitution of either S239D, I332E, or S239D/I332E. In some embodiments, the antibody described herein comprises an Fc domain having an amino acid substitution of either S239D, I332E, or S239D/I332E, and another Fc domain without an amino acid substitution of either S239D, I332E, or S239D/I332E.

In some embodiments, the first Fc domain and the second Fc domain contain a set of ADCC-enhanced variant substitutions (first Fc domain variant: second Fc domain variant) selected from the group including: S239: I332E; S239D: S239D; S239D: WT; S239D: S239D/I332E; S239D/I332E: WT; S239D/I332E: S239D; S239D/I332E: I332E; S239D/I332E: S239D/I332E; I332E: WT; I332E: I332E; I332E: S239D; I332E: S239D/I332E; WT: S239D; WT: I332E; WT: S239D/I332E, according to EU numbering. In some embodiments, monomer 1 and monomer 2 contain a set of ADCC-enhanced variant substitutions (monomer 1 monomer 2) selected from the group including: S239: I332E; S239D: S239D; S239D: WT; S239D: S239D/I332E; S239D/I332E: WT; S239D/I332E: S239D; S239D/I332E: 1332E; S239D/I332E: S239D/I332E; 1332E: WT; 1332E: 1332E; 1332E: S239D; 1332E: S239D/I332E; WT: S239D; WT: 1332E; WT: S239D/I332E, according to EU numbering.

In some embodiments, Fc domains with enhanced ADCC can further comprise one or more additional modifications at one or more of the following positions, including, but not limited to, 236, 243, 298, 299, or 330 in the CH2 domain, according to EU numbering. In some embodiments, the Fc variant domains comprise an amino acid substitution including, but not limited to: 236A, 243L, 298A, 299T, or 330L in the CH2 domain, according to EU numbering.

In some embodiments, an ADCC-enhanced Fc variant further includes, but is not limited, an amino acid substitution at one or more positions of the CH2 domain, according to EU numbering selected from the group including: position 236, 243, 298, 299, and 330. In some embodiments, an ADCC-enhanced Fc variant includes an amino acid substitution selected from the group including: 236A, 243L, 298A, 299T, 330L, 239D/332E, 236A/332E, 239D/332E/330L, 332E/330L, and any combination thereof in the CH2 domain, according to EU numbering. In some embodiments, the first Fc domain and/or the second Fc domain comprises an ADCC-enhanced Fc variant including, but not limited to, an amino acid substitution selected from the group including: 236A, 243L, 298A, 299T, 330L, 239D/332E, 236A/332E, 239D/332E/330L, 332E/330L, and any combination thereof in the CH2 domain, according to EU numbering, such that the Fc ADCC variant is the same in both Fc domain. Alternatively, the Fc ADCC variant is a different variant in each of the Fc domains.

Engineered antibodies comprising such ADCC-enhanced Fc variants can also have higher-affinity FcγRIIIa binding, thus resulting in stronger ADCC activity with NK cells. Bispecific antibodies having a variant Fc domain described herein can be useful and effective for NK cell-mediated killing of tumor cells.

### 2. Fc Variants to Increase Binding to FcγRIIIa/CD16A

There are additional Fc substitutions that find use in enhancing FcγRIIIa binding. In some embodiments, the Fc domains of the bispecific antibodies provided include one or more Fc domains having increased binding to FcγRIIIa as compared to human IgG1 produced in standard research and production cell lines. In some embodiments, the Fc variants with improved binding affinity to at least FcγRIIIa have amino acid substitution(s) selected from the group including: V264I/I332E, S298A, S298A/I332E, S298A/E333A/K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/1332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E298R, S324G, E272R, P227G, G236S, D221K, H224E, K246H, D249Y, R255Y, E258H, T260H, G281D, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering of the Fc domain. Additional Fc variants with enhanced binding affinity, specificity and/or avidity to FcγRIIIa are disclosed the specification and FIG. 41 of U.S. Pat. No. 8,188,231.

The described bispecific antibodies contain such Fc variants that provide enhanced effector function and substantial increases in affinity for FcγRIIIa. In some embodiments, the Fc variants improve binding to FcγRIIIa allotypes such as, for example, both V158 and F158 polymorphic forms of FcγRIIIa. The FcγR binding affinities of these Fc variants can be evaluated using assay recognized by those skilled in the art including, but not limited to, a Surface Plasmon Resonance (SPR) and/or a BLI binding assay (such as Biacore, Octet, or Carterra LSA).

### C. Fc Variants for Increasing Binding to FcRn

Provided herein are additional Fc substitutions that find use in increased binding to the FcRn receptor and increased serum half-life, as specifically disclosed in U.S. Ser. No. 12/341,769, hereby incorporated by reference in its entirety, including, but not limited to, N434S, N434A, M428L, V308F, V259I, M428L/N434S, M428L/N434A, V2591/V308F, Y436I/M428L, Y436I/N434S, Y436V/N434S, Y436V/M428L, M252Y/S254T/T256E, and V259I/V308F/M428L. Such modification may be included in one or both Fc domains of the subject antibody.

In some embodiments, additional Fc variants can increase serum half-life of a bispecific antibody compared to a parental Fc domain. In some embodiments, the Fc variants have one or more amino acid modifications (i.e., substitutions, insertions or deletions) at one or more of the following amino acid residues or positions selected from the group including: 234, 235, 238, 250, 252, 254, 256, 265, 272, 286, 303, 305, 307, 311, 312, 317, 322, 340, 356, 360, 362, 376, 378, 380, 382, 413, 424, 428, and 434, according to EU numbering of the Fc region.

In some embodiments, the Fc variants have one or more amino acid substitutions selected from the group including: 234F, 235Q, 250E, 250Q, 252T, 252Y, 254T, 256E, 428L, 428F, 434S, 434A, 428L/4345, 428L/434A, 252Y/254T/256E, 234F/235Q/252T/254T/256E/322Q, 250E/428F, 250E/428L, 250Q/428F, and 250Q/428L, according to EU numbering.

In some embodiments, antibodies described can include M428L/N434S or M428L/N434A substitutions in one or both Fc domains, which can result in longer half-life in serum. In more embodiments, a first Fc domain or a second Fc domain include M428L/N434S substitutions. In more embodiments, a first Fc domain and a second Fc domain include M428L/N434S substitutions. In certain embodiments, a first Fc domain or a second Fc domain include M428L/N434A substitutions. In certain embodiments, a first Fc domain and a second Fc domain include M428L/N434Asubstitutions. Such substitutions can result in longer half-life in serum of molecules comprising such.

### D. Fc Variants for Heterodimeric Antibodies

In some embodiments, the bispecific antibodies provided herein are heterodimeric bispecific antibodies that include two variant Fc domain sequences. Such variant Fc domains include amino acid modifications to facilitate the self-assembly and/or purification of the heterodimeric antibodies.

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibodies away from homodimers are further discussed in detailed below.

There are a number of suitable pairs of sets of heterodimerization skew variants. These variants come in “pairs” of “sets.” That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other; that is, these pairs of sets form an interface between the two monomers that encourages heterodimer formation and discourages homodimer formation, allowing the percentage of heterodimers that spontaneously form under biological conditions to be over 90%, rather than the expected 50% (25 homodimer A/A:50% heterodimer A/B:25% homodimer B/B).

### 1. Skew Variants

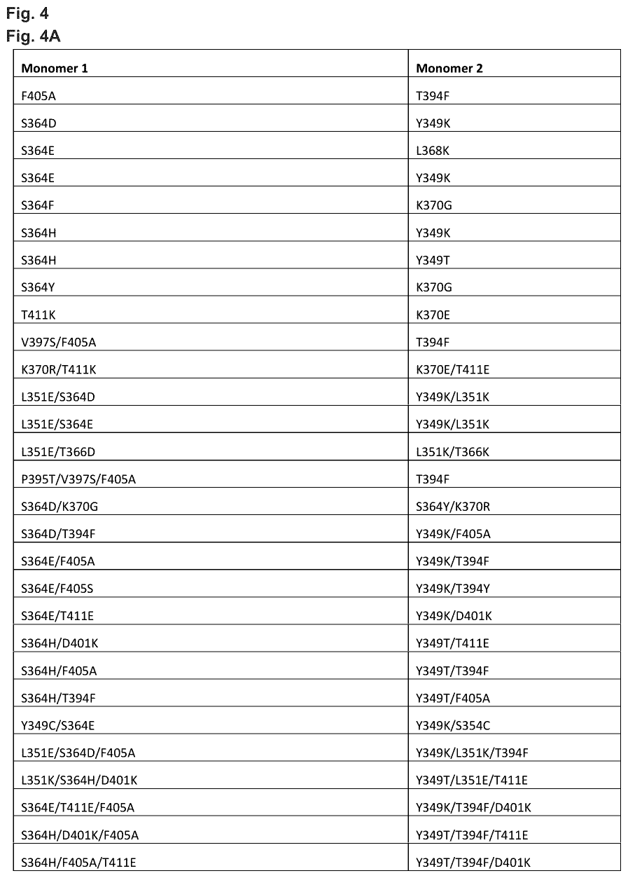

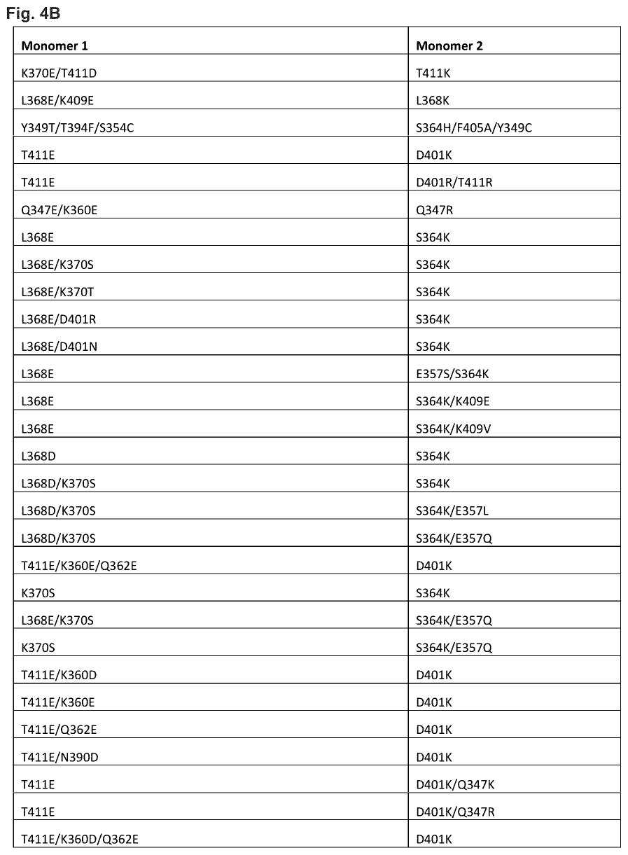

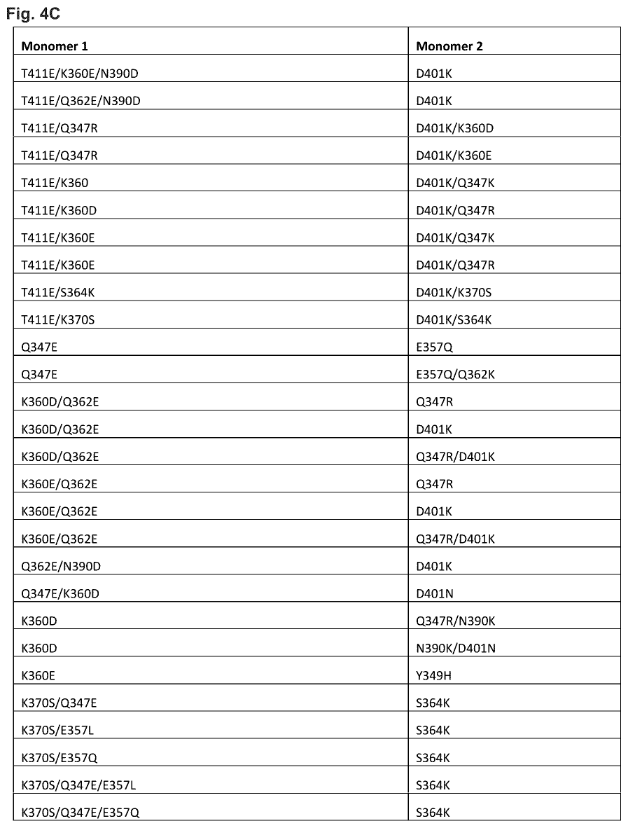

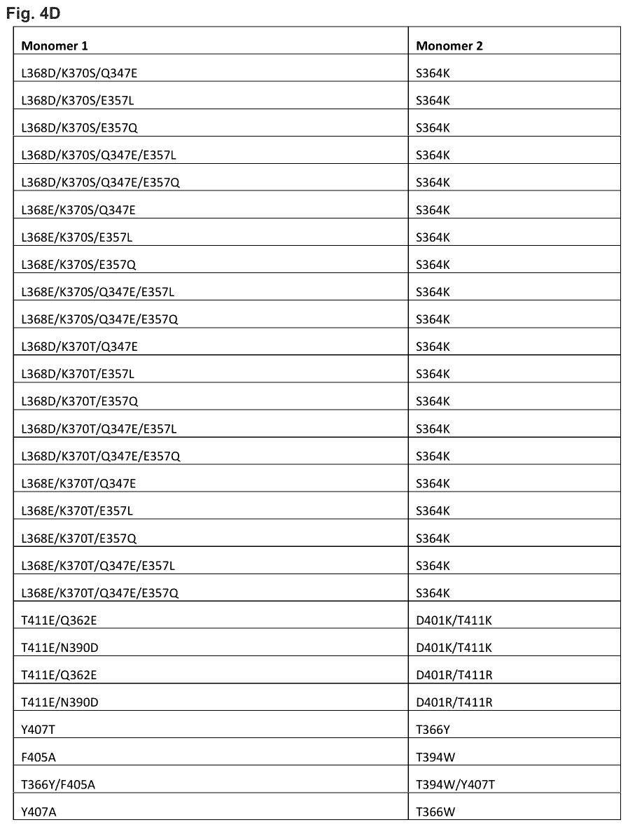

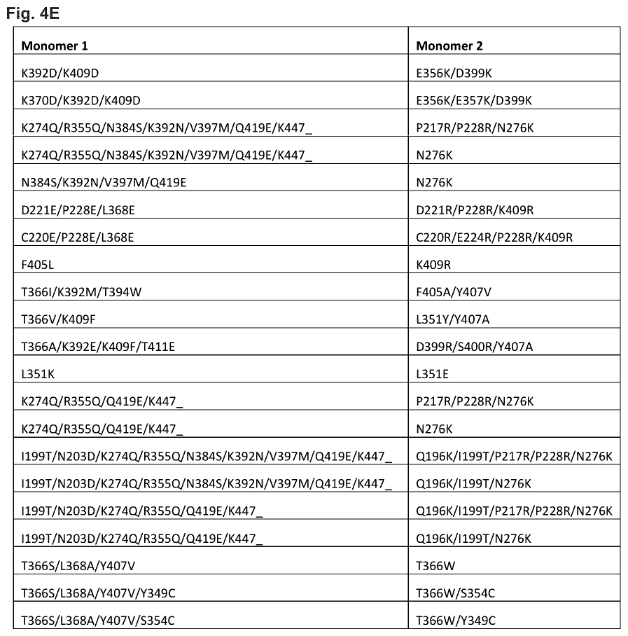

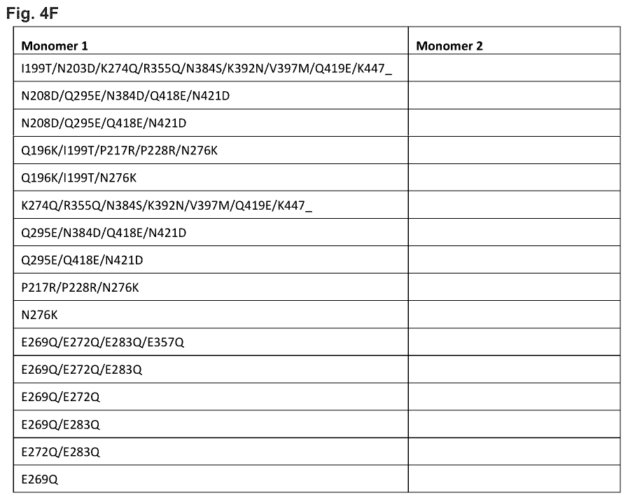

In some embodiments, the heterodimeric antibody includes skew (e.g., steric) variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of U.S. Publ. App. No. 2016/0355608, hereby incorporated by reference in its entirety and specifically for its disclosure of skew variants, as well as in FIGS. 4, 10, and 11 described herein.

Thus, suitable Fc heterodimerization variant pairs that will permit the formation of heterodimeric Fc regions are shown in the figures including FIGS. 4, 10, and 11. Thus, a first Fc domain has first Fc heterodimerization variants and the second Fc domain has second Fc heterodimerization variants selected from the pairs in FIGS. 4, 10, and 11.

One mechanism is generally referred to in the art as “knobs and holes,” referring to amino acid engineering that creates steric influences to favor heterodimeric formation and disfavor homodimeric formation can also optionally be used; this is sometimes referred to as “knobs and holes,” as described in U.S. Ser. No. 61/596,846, Ridgway et al., Protein Engineering 9(7):617 (1996); Atwell et al., J. Mol. Biol., 270:26 (1997); U.S. Pat. No. 8,216,805, all of which are hereby incorporated by reference in their entirety. The Figures identify a number of “monomer A-monomer B” pairs that rely on “knobs and holes”. In addition, as described in Merchant et al., Nature Biotech., 16:677 (1998), these “knobs and hole” mutations can be combined with disulfide bonds to skew formation to heterodimerization.

An additional mechanism that finds use in the generation of heterodimers is sometimes referred to as “electrostatic steering” as described in Gunasekaran et al., J. Biol. Chem., 285(25):19637 (2010), hereby incorporated by reference in its entirety. This is sometimes referred to herein as “charge pairs”. In this embodiment, electrostatics are used to skew the formation towards heterodimerization. As those in the art will appreciate, these variants may also have an effect on pI, and thus on purification, and thus could in some cases also be considered pI variants. However, as these were generated to force heterodimerization and were not used as purification tools, they are classified as “steric variants”. These include, but are not limited to, D221E/P228E/L368E paired with D221R/P228R/K409R (e.g., these are “monomer corresponding sets”) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets.” That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization skew variants are depicted in FIG. 11. Such skew variants include, but are not limited to: S364K/E357Q:L368D/K370S; L368D/K370S:S364K; L368E/K370S:S364K; T411E/K360E/Q362E:D401K; L368D/K370S:S364K/E357L; K370S:S364K/E357Q (EU numbering). In terms of nomenclature, the pair “S364K/E357Q:L368D/K370S” means that one of the monomers has the double variant set S364K/E357Q and the other has the double variant set L368D/K370S.

In exemplary embodiments, the heterodimeric antibody includes Fe heterodimerization variants as sets: S364K/E357Q:L368D/K370S; L368D/K370S:S364K; L368E/K370S:S364K; T411E/K360E/Q362E:D401K; L368D/K370S:S364K/E357L; K370S:S364K/E357Q; or a T366S/L368A/Y407V:T366W (optionally including a bridging disulfide, T366S/L368A/Y407V/Y349C:T366W/S354C or T366S/L368A/Y407V/S354C:T366W/Y349C) are all skew variant amino acid substitution sets of Fc heterodimerization variants. In an exemplary embodiment, the heterodimeric antibody includes a “S364K/E357Q:L368D/K370S” amino acid substitution set. In terms of nomenclature, the pair “S364K/E357Q:L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotypic variants, FcRn variants, ablation variants, etc. into one or both of the first and second Fc domains of the heterodimeric antibody. Further, individual modifications can also independently and optionally be included or excluded from the subject the heterodimeric antibody.

Additional monomer A and monomer B variants that can be combined with other variants, optionally and independently in any amount, such as pI variants outlined herein or other steric variants that are shown in FIG. 37 of US 2012/0149876, the figure and legend and SEQ ID NOs of which are incorporated expressly by reference herein.

In some embodiments, the steric variants outlined herein can be optionally and independently incorporated with any pI variant (or other variants such as, for example, Fc ADCC variants, FcRn variants, etc.) into one or both monomers, and can be independently and optionally included or excluded from the proteins of the antibodies described herein.

A subset of skew variants is “knobs in holes” (KIH) variants. Exemplary “knob-in-hole” variants are depicted in FIG. 7 of U.S. Pat. No. 8,216,805, which is incorporated herein by reference. Such “knob-in-hole” variants include, but are not limited to: an amino acid substitution at position 347, 349, 350, 351, 357, 366, 368, 370, 392, 394, 395, 397, 398, 399, 405, 407 and/or 409 of the CH3 constant domain of an IgG such as an IgG1, IgG2a, IgG2b, or IgG4 (Kabat numbering). In some embodiments, the “knob-in-hole “variants include, but are not limited to: an amino acid substitution at Y349, L351, E357, T366, L368, K370, N390, K392, T394, D399, S400, F405, Y407, K409, R409, T411, or any combination thereof of the CH3 domain of an IgG such as an IgG1, IgG2a, IgG2b, IgG4 (EU numbering). In some embodiments, the “knob-in-hole” variants include, but are not limited to: one or more amino acid substitutions including Y349D/E, L351D/K/Y, E357K, T366A/K/Y, L368E, K370E, N390D/K/R, K392E/F/L/M/R, T394W, D399K/R/W/Y, S400D/E/K/R, F405A/I/M/S/T/V/W, Y407A/Y, K409E/D/F, R409E/D/F, and T411D/E/K/N/Q/R/W.

In some embodiments, such variants include one or more amino acid substitutions including, but not limited to: Y349C, E357K, S354C, T366S, T366W, T366Y, L368A, K370E, T394S T394W, D399K, F405A, F405W, Y407A, Y407T, Y407V, R409D, T366Y/F405A, T394W/Y407T, T366W/F405W, T394S/Y407A, F405W/Y407A, and T366W/T394S (EU numbering). In some embodiments, these variants include knob:hole paired substitutions including, but not limited to: T366W:Y407V; S354C/T366W:Y349C/T366S/Y407V; Y349C/T366W:S354C/T366S/L368A/Y407V; Y349C/T366W/R409D/K370E:S354C/T366S/L368A/Y407V/D399K/E357K; R409D/K370E:D399K/E357K; T366W:T366S/L368A/Y407V; T366W/R409D/K370E:T366S/L368A/Y407V/D399K/E357K; T366W:T366S/L368A/Y407V; T366W/Y366Y:T366S/L368A/T394W/F405A/Y407V; Y349C/T366W:S354C/T366S/L368A/Y407V; Y349C/T366W/R409D/K370E:S354C/T366S/L368A/Y407V/D399K/E357K paired substitutions, according to EU numbering.

Additional exemplary “knob-in-hole” variants as described by the amino acid substitutions of the CH3 domains can be found in, for example, Carter et al., J. Immunol. Methods, 248(1-2):7-15 (2001), Merchant et al. Nat. Biotechnol. 16(7):677-81 (1998), Ridgway et al. Protein Eng. 9(7):617-2 (1996), and U.S. Pat. Nos. 8,216,805 and 10,287,352, the disclosures of which are herein incorporated by reference in their entireties.

### 2. pI (Isoelectric Point) Variants for Heterodimers

In some embodiments, the heterodimeric antibody includes purification variants that advantageously allow for the separation of heterodimeric antibody (e.g., anti-NKp46×anti-MICA/B bispecific antibody) from homodimeric proteins.

There are several basic mechanisms that can lead to ease of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins. Alternatively, some scaffold formats, such as the “1+1 Fab-scFv-Fc” format and the “2+1 Fab₂-scFv-Fc” format, also allows separation on the basis of size. As described above, it is also possible to “skew” the formation of heterodimers over homodimers using skew variants. Thus, a combination of heterodimerization skew variants and pI variants find particular use in the heterodimeric antibodies provided herein.

Additionally, as more fully outlined below, depending on the format of the heterodimeric antibody, pI variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc,” format can include charged scFv linkers (either positive or negative), that give a further pI boost for purification purposes. As will be appreciated by those in the art, some 1+1 Fab-scFv-Fc formats are useful with just charged scFv linkers and no additional pI adjustments, although the antibodies described herein do provide pI variants that are on one or both of the monomers, and/or charged domain linkers as well. In addition, additional amino acid engineering for alternative functionalities may also confer pI changes, such as Fc, FcRn and KO variants.

In subject heterodimeric antibodies for which pI is used as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B can be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (e.g., aspartic acid to lysine) or changing a charged residue to a neutral residue (e.g., loss of a charge; lysine to serine). A number of these variants are shown in the FIGS. 4, 5, and 10.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the antibodies described herein.

As will be appreciated by those in the art, the number of pI variants to be included on each or both monomer(s) to achieve good separation will depend in part on the starting pI of the components, for example in the 1+1 Fab-scFv-Fc and 2+1 Fab₂-scFv-Fc formats, the starting pI of the scFv and Fab(s) of interest. That is, to determine which monomer to engineer or in which “direction” (e.g., more positive, or more negative), the Fv sequences of the two target antigens are calculated and a decision is made from there. As is known in the art, different Fvs will have different starting pIs which are exploited in the antibodies described herein. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively, or in addition to isotypic substitutions, the possibility of immunogenicity resulting from the pI variants is significantly reduced by utilizing isosteric substitutions (e.g., Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

Exemplary combinations of pI variants are shown in FIGS. 4 and 5, and FIG. 30 of U.S. Publ. App. No. 2016/0355608, all of which are herein incorporated by reference in its entirety and specifically for the disclosure of pI variants. Preferred combinations of pI variants are shown in FIGS. 4, 5, and 10. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)₄ (SEQ ID NO: 1). However, as will be appreciated by those in the art, the first monomer includes a CH1 domain, including position 208. Accordingly, in constructs that do not include a CH1 domain (for example for fusion proteins that do not utilize a CH1 domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 4 and the other monomer has a charged linker (either in the form of a charged scFv linker because that monomer comprises an scFv or a charged domain linker, as the format dictates, which can be selected from those depicted in FIGS. 8 and 9.

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389, 392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

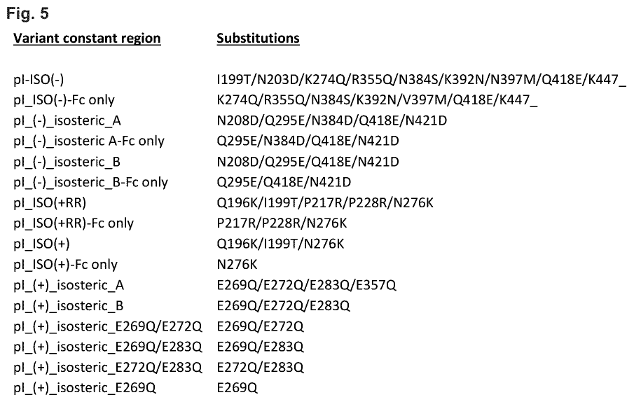

Preferred combinations of pI variants are shown in FIG. 5. As outlined herein and shown in the figures, these changes are shown relative to IgG1, but all isotypes can be altered this way, as well as isotype hybrids. In the case where the heavy chain constant domain is from IgG2 or IgG4, R133E and R133Q can also be used.

In one embodiment, for example in the FIGS. 25A-25E formats, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)₄ (SEQ ID NO: 1). However, as will be appreciated by those in the art, the first monomer includes a CH1 domain, including position 208. Accordingly, in constructs that do not include a CH1 domain (for example for antibodies that do not utilize a CH1 domain on one of the domains, for example in a dual scFv format or a “one-armed” format such as those depicted in FIGS. 25A-25C), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 4F and/or FIG. 5 and the other monomer has a charged linker (either in the form of a charged scFv linker because that monomer comprises an scFv or a charged domain linker, as the format dictates, which can be selected from those depicted in FIGS. 8 and 9.

### 3. Isotypic Variants

In addition, many embodiments of the antibodies described herein rely on the “importation” of pI amino acids at particular positions from one IgG isotype into another, thus reducing or eliminating the possibility of unwanted immunogenicity being introduced into the variants. A number of these are shown in FIG. 21 of U.S. Publ. App. No. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point. 4. Calculating pI:

The pI of each monomer can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of U.S. Publ. App. No. 2014/0370013. As discussed herein, which monomer to engineer is generally decided by the inherent pI of the Fv and scaffold regions. Alternatively, the pI of each monomer can be compared.

### 5. pI Variants that Also Confer Better FcRn In Vivo Binding

In the case where the pI variant decreases the pI of the monomer, they can have the added benefit of improving serum retention in vivo.

Although still under examination, Fc regions are believed to have longer half-lives in vivo, because binding to FcRn at pH 6 in an endosome sequesters the Fc (Ghetie and Ward, 1997, Immunol Today, 18(12): 592-598, entirely incorporated by reference). The endosomal compartment then recycles the Fc to the cell surface. Once the compartment opens to the extracellular space, the higher pH 7.4, induces the release of Fc back into the blood. In mice, Dall'Acqua et al. showed that Fc mutants with increased FcRn binding at pH 6 and pH 7.4 actually had reduced serum concentrations and the same half-life as wild-type Fc (Dall'Acqua et al., 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fe at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010, PEDS, 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### 6. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed herein.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

### 7. Additional Heterodimerization Variants

In some embodiments, the first Fc domain comprises one or more amino acid substitutions selected from the group including: L351Y, D399R, D399K, S400D, S400E, S400R, S400K, F405A, F405I, F405M, F405T, F405S, F405V, F405W, Y407A, Y407I, Y407L, Y407V, and any combination thereof, and the second Fc domain comprises one or more amino acid substitutions selected from the group including: T350V, T366A, T366I, T366L, T366M, T366Y, T366S, T366C, T366V, T366W, N390D, N390E, N390R, K392L, K392M, K392I, K392D, K392E, T394W, K409F, K409W, T411N, T411R, T411Q, T411K, T411D, T411E, T411W, and any combination thereof.

In some embodiments, other heterodimerization pair variants include, but are not limited to, amino acid substitutions of L234A/L235A: wildtype; L234A/L235A: L234K/L235K; L234D/L235E: L234K/L235K; E233A/L234D/L235E: E233A/L234R/L235R; L234D/L235E: E233K/L234R/L235R; E233A/L234K/L235A: E233K/L234A/L235K; E269Q/D270N: E269K/D270R; and WT: L235K/A327K of the CH2 domain, according to the EU numbering.

In some embodiments, the first and/or second Fe domains comprise one or more amino acid substitutions selected from the group including: S239D, D265S, S267D, E269K, S298A, K326E, A330L and 1332E. In certain instances, the Fc paired variants include, but are not limited to, S239D/D265S/I332E/E269K: S239D/D265S/S298A; S239D/K326E/A330L/I332E: S298A or S239D/K326E/A330L/I332E/E269K: S298A of the CH2 domain, according to EU numbering.

Additional descriptions of useful heterodimeric variants are disclosed in U.S. Pat. Nos. 9,732,155; 10,457,742 and 10,875,931 and U.S. Publ. App. Nos. 2021/0277150 and 2020/0087414, the disclosures of which, including the description of Fc domain variants are herein incorporated by reference in their entireties.

### E. Ablation Variants

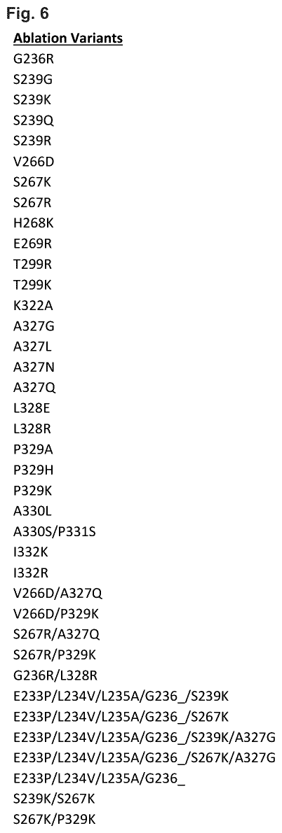

While in general NK engager multispecific antibodies retain at binding to CD16A (including “wild type” binding or increased binding to CD16A as outlined above), in some cases, surprisingly, NK engager activity can be seen even when binding to CD16A has been reduced or ablated. Accordingly, provided is another category of functional Fc variants to include are “FcγR ablation variants” or “Fc knock out (FcKO or KO)” variants. In these embodiments, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific antibodies that bind a target antigen monovalently it is generally desirable to ablate FcγRIIIa binding to eliminate or significantly reduce ADCC activity. Wherein one of the Fc domains comprises one or more Fcγ receptor ablation variants. These ablation variants are depicted in FIG. 6, and each can be independently and optionally included or excluded, with preferred aspects utilizing ablation variants selected from the group including G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fc receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

### F. Combination of Heterodimeric and Fc Variants

As will be appreciated by those in the art, all of the recited heterodimerization variants (including skew and/or pI variants) can be optionally and independently combined in any way, as long as they retain their “strandedness” or “monomer partition.” In addition, all of these variants can be combined into any of the heterodimerization formats.

In the case of pI variants, while embodiments finding particular use are shown in the Figures, other combinations can be generated, following the basic rule of altering the pI difference between two monomers to facilitate purification.

In addition, any of the heterodimerization variants, skew, and pI, are also independently and optionally combined with Fc ADCC variants, Fc variants, FcRn variants, or Fc ablation variants, as generally outlined herein.

Exemplary combination of variants that are included in some embodiments of the heterodimeric 1+1 Fab×scFv, 1+1 empty×Fab-scFv, 2+1 Fab×Fab-scFv, 2+1 Fab2×scFv, and 2+1 mAb-scFv format antibodies are included in FIG. 4 and FIGS. 10-16. In certain embodiments, the antibody is a heterodimeric 1+1 Fab×scFv, 1+1 empty×Fab-scFv, 2+1 Fab×Fab-scFv, 2+1 Fab2×scFv, and 2+1 mAb-scFv formats format antibody as shown in FIGS. 25A-25E.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

### G. Afucosylated Fc Domains

In some embodiments, the increased binding of a Fc domain to CD16A is the result of producing the NKE in a cell line that reduces or eliminates the incorporation of fucose into the glycosylation of the NKE. See, for example, Pereira et al., MAbs (2018) 10(5):693-711.

In some embodiments, antibodies comprising Fc domains described are produced in a host cell such that the Fc domains have reduced fucosylation or no fucosylation compared to a parental Fc domain. In some instances, antibodies described are produced in a genetically modified host cell, wherein the genetic modification to the host cell results in the overexpression of β(1,4)-N-acetylglucosaminyltransferase III (GnTIII), a glycosyltransferase catalyzing the formation of bisected oligosaccharides, which are generally also non-fucosylated. N-glycosylation of the Fc domain can play a role in binding to FcγR; and afucosylation of the N-glycan can increase the binding capacity of the Fc domain to FcγRIIIa. As discussed in further detail above, an increase in FcγRIIIa binding can enhance ADCC, which can be advantageous in certain antibody therapeutic applications in which cytotoxicity is desirable.

In some embodiments, an Fc domain is engineered such that it has reduced fucosylation or no fucosylation, compared to a parental Fc domain. In the context of an Fc domain, the terms “afucosylation,” “afucosylated,” “defucosylation,” and “defucosylated” are used interchangeably, and generally refer to the absence or removal of core-fucose from the N-glycan attached to the CH2 domain of an Fc domain. For instance, an afucosylated antibody lacks core fucosylation in the Fc domain. As used herein, the phrase “a low level of fucosylation” or “reduced fucosylation” generally refers to an overall fucosylation level in a specific Fc domain that is no more than about 10.0%, no more than 5.0%, no more than 2.5%, no more than 1.0%, no more than about 0.5%, no more than 0.25%, no more than about 0.1%, or no more than 0.01%, compared to the fucosylation level of parental Fc domain. The term “% fucosylation” generally refers to the level of fucosylation in a specific Fc domain compared to that of a parental Fc domain. The % fucosylation can be measured according to any suitable method known in the relevant art, such as, for example, by mass spectrometry (MS), HPLC-Chip Cube MS (Agilent), and reverse phase-HPLC.

In some embodiments, a particular level of fucosylation is desired. In some embodiments, a Fc variant is provided, wherein the Fc variant comprises a particular level of afucosylation. In some further embodiments, the fucosylation level of the Fc variant is no more than about 10.0%, no more than about 9.0%, no more than about 8.0%, no more than about 7.0%, no more than about 6.0%, no more than about 5.0%, no more than about 4.0%, no more than about 3.0%, no more than about 2.0%, no more than about 1.5%, no more than about 1.0%, no more than about 0.5%, no more than 0.25%, no more than about 0.1%, or no more than 0.01%, compared to that of a parental Fc domain.

In some embodiments, antibodies comprising afucosylated Fe domains can be enriched (to obtain a particular level of afucosylation) by affinity chromatography using resins conjugated with a fucose binding moiety, such as, for example, an antibody or lectin specific for fucose, with some embodiments finding particular utility when fucose is present in a 1-6 linkage (see, e.g., Kobayashi et al., 2012, J. Biol. Chem. 287:33973-82).

In some embodiments, the fucosylated species of the Fc domain can be separated from the afucosylated species of the Fc domain (to obtain a particular level of afucosylation) using an anti-fucose specific antibody in an affinity column. Alternatively, or in addition to, afucosylated species can be separated from fucosylated species based on the differential binding affinity to FcγRIIIa using affinity chromatography (again, to obtain a particular level of afucosylation).

### H. Useful Formats of the Invention

As will be appreciated by those in the art, and discussed more fully below, the heterodimeric bispecific antibodies provided herein can take on a wide variety of configurations, as are generally depicted in FIGS. 25A-25E. Some figures depict “single ended” configurations, where there is one type of specificity on one “arm” of the molecule and a different specificity on the other “arm.” Other figures depict “dual ended” configurations, where there is at least one type of specificity at the “top” of the molecule and one or more different specificities at the “bottom” of the molecule. Thus, in some embodiments, the antibodies described herein are directed to novel immunoglobulin compositions that co-engage a first antigen and a second antigen that are different.

As will be appreciated by those in the art, the heterodimeric formats of the antibodies described herein can have different valences (e.g., bivalent, trivalent, etc.), as well as specificity (e.g., bispecific). That is, in some embodiments, heterodimeric antibodies of the antibodies described herein can be bivalent and bispecific, wherein one target antigen (for instance and merely as an example, NKp46) is bound by a first binding domain and the other target antigen (for instance and merely as an example, MICA/B) is bound by a second binding domain. In other embodiments, the heterodimeric antibodies can be trivalent and bispecific, wherein the first target antigen is bound by two binding domains (i.e., a first binding domain and a second binding domain) and the second target antigen is bound by another and different binding domain. As will be appreciated by those skilled in the relevant art, the heterodimeric formats of the antibodies described herein can incorporate an ABD as described herein (i.e., an anti-MICA/B ABD or an anti-NKp46 ABD) in conjunction with an ABD capable of specifically binding a separate target antigen, wherein the separate target antigen can be any number of target antigens relevant to cancer therapy. In some embodiments, the target antigen is a tumor target antigen. It is expressly contemplated that such an antibody comprising: (i) an anti-MICA/B ABD or an anti-NKp46 ABD, and (ii) an anti-target antigen binding domain, can be configured in any number of different formats with varying valences and/or specificity. In some further embodiments, the target antigen comprises a tumor target antigen.

The antibodies described herein utilize anti-MICA/B ABDs in combination with anti-NKp46 ABDs. As will be appreciated by those in the art, any collection of anti-MICA/B CDRs, anti-MICA/B variable light and variable heavy domains, Fabs, and scFvs as described herein, and depicted in any of the figures can be used. Similarly, any of the anti-NKp46 ABDs can be used, whether CDRs, variable light and variable heavy domains, Fabs, and scFvs as described herein, and depicted in any of the Figures can be used, optionally and independently combined in any combination. Alternatively, or in addition to, the antibodies described herein can utilize (i) an anti-MICA/B ABD or an anti-NKp46 ABD as described herein, and (ii) an anti-target antigen binding domain. In some further embodiments, the target antigen comprises a tumor target antigen.

In some embodiments of a bispecific antibody having one of the following formats: (i) a 1+1 Fab×scFv format, (ii) a 2+1 Fab×Fab-scFv format, (iii) a 2+1 mAb-scFv format, (iv) a 1+1 Empty×Fab-scFv format, or (v) a 2+1 Fab2×scFv format, the bispecific antibody comprises an anti-MICA/B ABD as described herein and an second ABD that binds to a different target antigen. In some further embodiments, the different target antigen comprises a tumor target antigen.

### 1. 1+1 Fab×scFv Format

One heterodimeric scaffold that finds particular use in the antibodies described herein is the “1+1 Fab×scFv” format (also referred to herein as the “1+1 Fab-scFv-Fc” or “bottle-opener” format), as is shown in FIG. 25A. In this embodiment, one heavy chain monomer of the antibody contains a single chain Fv (“scFv,” as described below) and an Fc domain. The scFv includes a variable heavy domain (VH1) and a variable light domain (VL1), wherein the VH1 is attached to the VL1 using an scFv linker that can be charged (see, e.g., FIG. 8; SEQ ID NOs: 23-48). The scFv is attached to the heavy chain using a domain linker (see, e.g., FIG. 9; SEQ ID NOs: 49-77). The other heavy chain monomer is a “regular” heavy chain (V_{H}-CH1-hinge-CH2-CH3). The 1+1 Fab×scFv format also includes a light chain that interacts with the V_{H}-CH1 to form a Fab. This structure is sometimes referred to herein as the “bottle-opener” format due to a rough visual similarity to a bottle-opener. The two heavy chain monomers are brought together by the use of amino acid variants (e.g., heterodimerization variants, discussed above) in the constant regions (e.g., the Fc domain, the CH1 domain, and/or the hinge region) that promote the formation of heterodimeric antibodies, as is described more fully below.

There are several distinct advantages to the present “1+1 Fab×scFv” format. As is known in the art, antibody analogs relying on two scFv constructs often have stability and aggregation problems, which can be alleviated in the antibodies described herein by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

Many of the embodiments outlined herein rely in general on the 1+1 Fab×scFv or “bottle opener” format antibody that comprises a first monomer comprising an scFv, comprising a variable heavy and a variable light domain, covalently attached using an scFv linker (charged, in many but not all instances), where the scFv is covalently attached to the N-terminus of a first Fc domain, usually through a domain linker. The domain linker can be either charged or uncharged, and exogenous or endogenous (e.g., all or part of the native hinge domain). Any suitable linker can be used to attach the scFv to the N-terminus of the first Fc domain. In some embodiments, the domain linker is chosen from the domain linkers in FIG. 9. The second monomer of the 1+1 Fab×scFv format or “bottle opener” format is a heavy chain, and the composition further comprises a light chain.

In general, in many preferred embodiments, the scFv is the domain that binds to NKp46, and the Fab forms a MICA/B binding domain. However, in several preferred embodiments, the scFv is the domain that binds to MICA/B, and the Fab forms a NKp46 binding domain. An exemplary anti-NKp46×anti-MICA/B bispecific antibody in the 1+1 Fab×scFv is depicted in FIG. 25A. Exemplary anti-NKp46×anti-MICA/B bispecific antibodies in the 1+1 Fab-scFv-Fc format are depicted in FIGS. 31A and 31B.

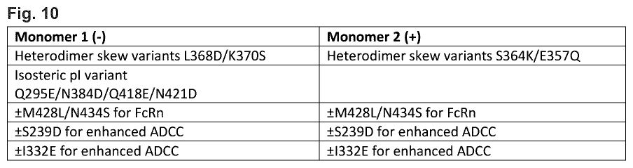

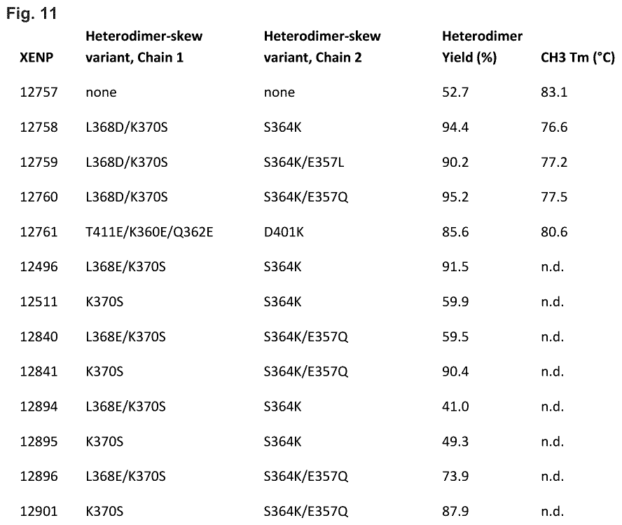

In addition, the Fc domains of the antibodies described herein generally include Fc ADCC variants (including, but not limited to, those shown in FIGS. 7 and 10), skew variants (e.g., a set of amino acid substitutions as shown in FIGS. 4, 10, and 11, with particularly useful skew variants being selected from the group including: (i) S364K/E357Q:L368D/K370S, (ii) L368D/K370S:S364K, (iii) L368E/K370S:S364K, (iv) T411T/E360E/Q362E:D401K, (v) L368D/K370S:S364K/E357L, (vi) K370S:S364K/E357Q, (vii) T366S/L368A/Y407V:T366W, and (viii) T366S/L368A/Y407V/Y349C:T366W/S354C), optionally ablation variants (including those shown in FIG. 6), optionally charged scFv linkers (including those shown in FIG. 8; see, e.g., SEQ ID NOs: 23-48), and the heavy chain comprises pI variants (including those shown in FIGS. 4, 5, and 10).

In certain embodiments the 1+1 Fab×scFv scaffold format includes a first monomer that includes a scFv-domain linker-CH2-CH3 monomer, a second monomer that includes a first variable heavy domain-CH1-hinge-cH2-CH3 monomer, and a third monomer that includes a first variable light domain. In some embodiments, the CH2-CH3 of the first monomer is a first variant Fc domain and the CH2-CH3 of the second monomer is a second variant Fc domain. In some embodiments, the scFv includes a scFv variable heavy domain and a scFv variable light domain that form a NKp46 binding moiety. In other embodiments, the scFv includes a scFv variable heavy domain and a scFv variable light domain that form a MICA/B binding moiety. In certain embodiments, the scFv variable heavy domain and scFv variable light domain are covalently attached using an scFv linker (charged, in many but not all instances; see, e.g., FIG. 8 and SEQ ID NOs: 42-48)). In some embodiments, the first variable heavy domain and first variable light domain form a MICA/B binding domain. In other embodiments, the first variable heavy domain and first variable light domain form a NKp46 binding domain.

Any suitable NKp46 ABD and/or MICA/B ABD can be included in the 1+1 Fab×scFv format antibody, including those provided herein. NKp46 binding domain sequences finding particular use in these embodiments include, but are not limited to: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, or variants thereof (see, e.g., FIGS. 23 and 24). MICA/B ABDs that are of particular use in these embodiments include, but are not limited to, V_{H} and V_{L} domains selected from V_{H}/V_{L} pairs selected from the group including: (i) SEQ ID Nos: 239-246 for D94837_1E11_1 [MICA/B]_H0_D94837_E11_1 [MICA/B]_L0, (ii) SEQ ID Nos: 247-254 for D94837_1E11_1 [MICA/B]_H1_D94837_E11_1 [MICA/B]_L1, (iii) SEQ ID Nos: 255-262 for D94837_E11_1 [MICA/B]_H1_D94837_E11_1 [MICA/B]_L2, (iv) SEQ ID Nos: 263-270 for D94837_E11_1 [MICA/B]_H2_D94837_E11_1 [MICA/B]_L1, (v) SEQ ID Nos: 271-278 for D94837_E11_1 [MICA/B]_H2_D94837_E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0, or variants thereof (see, e.g., FIGS. 21 and 22).

Similarly, any suitable MICA/B ABD and/or NKp46 ABD can be included in the 1+1 Fab×scFv format antibody, including those provided herein. MICA/B binding domain sequences finding particular use in these embodiments include, but are not limited to: (i) SEQ ID Nos: 239-246 for D94837_1E11_1 [MICA/B]_H0_D94837_1E11_1 [MICA/B]_L0, (ii) SEQ ID Nos: 247-254 for D94837_1E11_1 [MICA/B]_H1_D94837_1E11_1 [MICA/B]_L1, (iii) SEQ ID Nos: 255-262 for D94837_E11_1 [MICA/B]_H1_D94837_E11_1 [MICA/B]_L2, (iv) SEQ ID Nos: 263-270 for D94837_E11_1 [MICA/B]_H2_D94837_E11_1 [MICA/B]_L1, (v) SEQ ID Nos: 271-278 for D94837_1E11_1 [MICA/B]_H2_D94837_1E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0, or variants thereof (see, e.g., FIGS. 21 and 22). NKp46 ABDs that are of particular use in these embodiments include, but are not limited to, V_{H} and V_{L} domains selected from V_{H}/V_{L} pairs selected from the group including: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, or variants thereof (see, e.g., FIGS. 23 and 24).

In particular embodiments, the αNKp46 V_{H}/V_{L} pairs are selected from the group including: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, or a variant thereof (see, e.g., FIGS. 23 and 24).

In particular embodiments, the αMICA/B V_{H}/V_{L} pairs are selected from the group including: (i) SEQ ID Nos: 239-246 for D94837_E11_1 [MICA/B]_H0_D94837_1E11_1 [MICA/B]_L0, (ii) SEQ ID Nos: 247-254 for D94837_E11_1 [MICA/B]_H1_D94837_1E11_1 [MICA/B]_L1, (iii) SEQ ID Nos: 255-262 for D94837_1E11_1 [MICA/B]_H1_D94837_E11_1 [MICA/B]_L2, (iv) SEQ ID Nos: 263-270 for D94837_1E11_1 [MICA/B]_H2_D94837_E11_1 [MICA/B]_L1, (v) SEQ ID Nos: 271-278 for D94837_1E11_1 [MICA/B]_H2_D94837_E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0, or a variant thereof (see, e.g., FIGS. 21 and 22).

In some embodiments, the 1+1 Fab×scFv format includes Fc ADCC variants, skew variants, pI variants, and/or ablation variants. Accordingly, some embodiments include 1+1 Fab×scFv formats that comprise: (i) a first monomer (the “scFv monomer”) that comprises a charged scFv linker (with the “+H” sequence of FIG. 8 (i.e., SEQ ID NO: 32) being preferred in some embodiments), the skew variants S364K/E357Q, the ablation variants E233P/L234V/L235A/G236del/S267K, and an scFv that binds to a first target antigen as outlined herein, (ii) a second monomer (the “Fab monomer”) that comprises the skew variants L368D/K370S, the pI variants N208D/Q295E/N384D/Q418E/N421D, the ablation variants E233P/L234V/L235A/G236del/S267K, and a variable heavy domain (V_{H}), and (iii) a light chain that includes a variable light domain (V_{L}) and a constant light domain (C_{L}), wherein numbering is according to EU numbering. In some embodiments, the first target antigen is NKp46, and the first variable heavy domain and the first variable light domain make up a MICA/B binding moiety. In other embodiments, the first target antigen is MICA/B, and the first variable heavy domain and the first variable light domain make up a NKp46 binding moiety. NKp46 binding domain sequences finding particular use in these embodiments include, but are not limited to: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, or variants thereof (see, e.g., FIGS. 23 and 24). MICA/B binding domain sequences finding particular use in these embodiments include, but are not limited to: (i) SEQ ID Nos: 239-246 for D94837_1E11_1 [MICA/B]_H0_D94837_E11_1 [MICA/B]_L0, (ii) SEQ ID Nos: 247-254 for D94837_1E11_1 [MICA/B]_H1_D94837_1E11_1 [MICA/B]_L1, (iii) SEQ ID Nos: 255-262 for D94837_1E1_1 [MICA/B]_H1_D94837_E11_1 [MICA/B]_L2, (iv) SEQ ID Nos: 263-270 for D94837_E11_1_[MICA/B]_H2_D94837_E11_1 [MICA/B]_L1, (v) SEQ ID Nos: 271-278 for D94837_1E11_1 [MICA/B]_H2_D94837_E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0, or variants thereof (see, e.g., FIGS. 21 and 22).

In some embodiments, the 1+1 Fab×scFv format includes skew variants (see, e.g., FIGS. 4, 10, and 11), pI variants (see, e.g., FIGS. 4, 5, and 10), ablation variants (see, e.g., FIG. 6, and/or FcRn variants (see, e.g., FIG. 10). Accordingly, some embodiments include 1+1 Fab×scFv formats that comprise: (i) a first monomer (the “scFv monomer”) that comprises a charged scFv linker (with the “+H” sequence of FIG. 8 (i.e., SEQ ID NO: 32) being preferred in some embodiments), the skew variants S364K/E357Q, the ablation variants E233P/L234V/L235A/G236del/S267K, the FcRn variants M428L/N434S and an scFv that binds to a first target antigen as outlined herein, (ii) a second monomer (the “Fab monomer”) that comprises the skew variants L368D/K370S, the pI variants N208D/Q295E/N384D/Q418E/N421D, the ablation variants E233P/L234V/L235A/G236del/S267K, the FcRn variants M428L/N434S, and a variable heavy domain, and (iii) a light chain that includes a variable light domain (V_{L}) and a constant light domain (C_{L}), wherein numbering is according to EU numbering. In some embodiments, the first target antigen is NKp46, and the first variable heavy domain and the first variable light domain make up a MICA/B binding moiety. In other embodiments, the first target antigen is MICA/B, and the first variable heavy domain and the first variable light domain make up a NKp46 binding moiety. NKp46 binding domain sequences finding particular use in these embodiments include, but are not limited to: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, or variants thereof (see, e.g., FIGS. 23 and 24). MICA/B binding domain sequences finding particular use in these embodiments include, but are not limited to: (i) SEQ ID Nos: 239-246 for D94837_E11_1 [MICA/B]_H0_D94837_1E11_1 [MICA/B]_L0, (ii) SEQ ID Nos: 247-254 for D94837_E11_1 [MICA/B]_H1_D94837_1E11_1 [MICA/B]_L1, (iii) SEQ ID Nos: 255-262 for D94837_1E11_1 [MICA/B]_H1_D94837_E11_1 [MICA/B]_L2, (iv) SEQ ID Nos: 263-270 for D94837_1E11_1 [MICA/B]_H2_D94837_1E11_1 [MICA/B]_L1, (v) SEQ ID Nos: 271-278 for D94837_1E11_1 [MICA/B]_H2_D94837_E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0, or variants thereof (see, e.g., FIGS. 21 and 22).

Particularly useful NKp46 and MICA/B sequence combinations for use with the 1+1 Fab×scFv include, for example, those disclosed in FIGS. 31A and 31B.

FIGS. 12-15 show exemplary Fc domain sequences that are useful in the 1+1 Fab×scFv format antibodies. The “monomer 1” sequences depicted in FIGS. 12-15 typically refer to the Fc domain of the “Fab-Fc heavy chain,” and the “monomer 2” sequences refer to the Fc domain of the “scFv-Fc heavy chain.” In some embodiments, the heterodimeric Fc backbones of the first and second monomers of the 1+1 Fab×scFv format include a backbone pair set forth in FIGS. 12-15.

In some embodiments, one or both of the Fc domains comprising the heterodimeric Fc backbone pairs are configured such that one or both of the Fc domains have wildtype FcγR effector function. In some embodiments, the heterodimeric Fc backbone pairs have wildtype FcγR effector function, including, but not limited to, those pairs of: SEQ ID NOs: 112-133, as shown in FIGS. 13A-13C.

In some embodiments, one or both of the Fc domains comprising the heterodimeric Fc backbone pairs include amino acid substitutions such that one or both of the Fc domains have increased ADCC activity. In certain embodiments, the amino acid substitutions include one or more of: S239D, 1332E, or S239D/I332E, or any of the Fc ADCC variants shown in FIGS. 7 and 10. In some instances, only monomer 1 or monomer 2 of the heterodimeric Fc backbone pairs include amino acid substitutions resulting in increased ADCC activity. In such an instance, the pair of heterodimeric Fc backbones may be referred to as an asymmetric, ADCC-enhanced heterodimeric Fc backbone pair. In other instances, both monomers (i.e., monomer 1 and monomer 2) of the heterodimeric Fc backbone pairs independently include one or more amino acid substitutions conferring an increased ADCC activity. In such an instance, the pair of heterodimeric Fc backbones may be referred to as a symmetric, ADCC-enhanced heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) includes amino acid variants conferring an increase in ADCC activity. In some instances, monomer 1 and monomer 2 can contain identical amino acid substitutions (e.g., both monomers contain a S239D substitution, an I332E substitution, or both substitutions are present on both monomers). In other instances, monomer 1 and monomer 2 can contain at least one amino acid substitution that is the same (e.g., monomer 1 may contain the S239D/I332E substitutions, and monomer 2 contains the S239D substitution and is WT with respect to the 332 residue (or the reverse where monomer 2 is WT with respect to the 239 residue and contains the 1332E substitution); monomer 2 may contain the S239D/I332E substitutions, and monomer 1 contains the S239D substitution and is WT with respect to the 332 residue (or the reverse where monomer 1 is WT with respect to the 239 residue and contains the 1332E substitution); etc.). In still other instances, monomer 1 and monomer 2 can contain amino acid substitutions that are unique to each monomer (e.g., S239D on monomer 1 and 1332E on monomer 2, or the reverse). In some embodiments, the heterodimeric Fc backbone pairs have increased ADCC activity, including, but not limited to, those pairs of: SEQ ID NOs: 134-163, as shown in FIGS. 14A-14C.

In some embodiments, one or both of the Fc domains comprising the heterodimeric Fc backbone pairs include amino acid substitutions such that one or both of the Fc domains have improved serum half-life. In certain embodiments, the amino acid substitutions include one or more of: M428L, N434S, N434A, M428L/N434S, M252Y/S254T/T256E, or M428L/N434A. In some instances, only monomer 1 or monomer 2 of the heterodimeric Fc backbone pairs include amino acid substitutions resulting in improved serum half-life. In such an instance, the pair of heterodimeric Fc backbones may be referred to as an asymmetric, Xtend heterodimeric Fc backbone pair. In other instances, both monomers (i.e., monomer 1 and monomer 2) of the heterodimeric Fc backbone pairs independently include one or more amino acid substitutions conferring an increase serum half-life. In such an instance, the pair of heterodimeric Fc backbones may be referred to as a symmetric, Xtend heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants conferring an increased serum half-life. In some instances, monomer 1 and monomer 2 can contain identical amino acid substitutions. In other instances, monomer 1 and monomer 2 can contain at least one amino acid substitution that is the same (e.g., both monomers contain the M428L substitution, one monomer further contains the N434S substitution, and the other monomer contains the N434A mutation). In some instances, monomer 1 and monomer 2 can contain amino acid substitutions that are unique to each monomer (e.g., N434S on one monomer, and N434A on the other monomer).

In some embodiments, one or both of the Fe domains comprising the heterodimeric Fc backbone pairs include amino acid substitutions such that one or both of the Fc domains have increased ADCC activity and improved serum half-life. In certain embodiments, the amino acid substitutions include: (i) one or more of amino acid substitutions selected from the group including: S239D, 1332E, and S239D/I332E, or any of the Fc ADCC variants shown in FIGS. 7 and 10, and (ii) one or more amino acid substitutions selected from the group including: M428L, N434S, N434A, M428L/N434S, M252Y/S254T/T256E, and M428L/N434A. In some instances, only monomer 1 or monomer 2 of the heterodimeric Fc backbone pairs include amino acid substitutions resulting in increased ADCC and increased serum half-life. In such an instance, the pair of heterodimeric Fc backbones may be referred to as an asymmetric, ADCC-enhanced heterodimeric Fc backbone pair with Xtend. In other instances, both monomers (i.e., monomer 1 and monomer 2) of the heterodimeric Fc backbone pairs independently comprise amino acid substitutions resulting in increased ADCC and increased serum half-life. In such an instance, the pair of heterodimeric Fc backbones may be referred to as a symmetric, ADCC-enhanced heterodimeric Fc backbone pair with Xtend. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants conferring an increase in ADCC activity and improved serum half-life. In some instances, monomer 1 and monomer 2 can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased ADCC activity and increased serum-half-life, including, but not limited to, those pairs of: SEQ ID NOs: 164-193, as shown in FIGS. 15A-15C.

In some embodiments, one or both of the Fc domains comprising the heterodimeric Fc backbone pairs include amino acid substitutions such that one or both of the Fc domains have an ablated FcγR function. Nonlimiting examples of amino acid substitutions (and combinations thereof) conferring an ablated FcγR function are shown in, for example, FIG. 6. In some instances, only monomer 1 or monomer 2 of the heterodimeric Fc backbone pairs include amino acid substitutions that result in an ablated FcγR function (see, e.g., FIG. 6). In such an instance, the pair of heterodimeric Fc backbones may be referred to as an asymmetric, FcKO heterodimeric Fc backbone pair. In other instances, both monomers (i.e., monomer 1 and monomer 2) of the heterodimeric Fc backbone pairs independently comprise one or more amino acid substitutions resulting in an ablated FcγR function (see, e.g., FIG. 6). In such an instance, the pair of heterodimeric Fc backbones may be referred to as a symmetric, FcKO heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants resulting in an ablated FcγR function. In some embodiments, the heterodimeric Fc backbone pairs include amino acid substitutions such that the Fc domains have an ablated FcγR effector function, including, but not limited to, those pairs of: SEQ ID NOs: 78-111, as shown in FIGS. 12A-12E.

Further, FIG. 19 provides useful C_{L} sequences that can be used with this format (see, e.g., SEQ ID NOs: 237-238).

In some embodiments, any of the V_{H} and V_{L} sequences depicted herein (including all V_{H} and V_{L} sequences depicted in the figures and sequence listing, including those directed to NKp46) can be added to the bottle opener backbone formats of FIGS. 12-15 as the “Fab side,” using any of the anti-NKp46 scFv sequences shown in the Figures and sequence listing. Alternatively, any of the V_{H} and V_{L} sequences depicted herein (including all V_{H} and V_{L} sequences depicted in the figures and sequence listing, including those directed to MICA/B) can be added to the bottle opener backbone formats of FIGS. 12-15 as the “Fab side,” using any of the anti-MICA/B scFv sequences shown in the Figures and sequence listing.

For the 1+1 Fab×scFv backbones depicted in FIGS. 12-15, NKp46 binding domain sequences finding particular use in these embodiments include, but are not limited to, NKp46 binding domain 2C10A3.372[NKp46]_H1_2C10A3.372[NKp46]_L1 (SEQ ID NOs: 719 and 723), and NKp46-A[NKp46]_H_NKp46-A[NKp46]_L (SEQ ID NOs: 727 and 731) (see, e.g., FIGS. 23 and 24) attached as the scFv side of the backbones shown in FIGS. 12-15. Alternatively, MICA/B binding domain sequences finding particular use in these embodiments include, but are not limited to, MICA/B binding domain (i) D94837_E11_1 [MICA/B]_H0_D94837_1E11_1 [MICA/B]_L0 (SEQ ID NOs: 239-246), (ii) D94837_E11_1 [MICA/B]_H1_D94837_1E11_1 [MICA/B]_L1 (SEQ ID NOs: 247-254), (iii) D94837_E11_1 [MICA/B]_H1_D94837_1E11_1 [MICA/B]_L2 (SEQ ID NOs: 255-262), (iv) D94837_E11_1 [MICA/B]_H2_D94837_1E11_1 [MICA/B]_L1 (SEQ ID NOs: 263-270), (v) D94837_E11_1 [MICA/B]_H2_D94837_1E11_1 [MICA/B]_L2 (SEQ ID NOs: 271-278), (vi) 2E5 [MICA/B]_H0_2E5 [MICA/B]_L0 (SEQ ID NOs: 279-286), (vii) 2E5 [MICA/B]_H1_2E5 [MICA/B]_L1 (SEQ ID NOs: 287-294), (viii) 2E5 [MICA/B]_H1_2E5 [MICA/B]_L2 (SEQ ID NOs: 295-302), (ix) 2E5 [MICA/B]_H2_2E5 [MICA/B]_L1 (SEQ ID NOs: 303-310), (x) 2E5 [MICA/B]_H2_2E5 [MICA/B]_L2 (SEQ ID NOs: 311-318), (xi) D94852_2E12 [MICA/B]_H0_D94852_2E12 [MICA/B]_L0 (SEQ ID NOs: 319-326), (xii) D94852_2E12 [MICA/B]_H1_D94852_2E12 [MICA/B]_L1 (SEQ ID NOs: 327-334), (xiii) D94852_2E12 [MICA/B]_H1_D94852_2E12 [MICA/B]_L2 (SEQ ID NOs: 335-342), (xiv) D94852_2E12 [MICA/B]_H2_D94852_2E12 [MICA/B]_L1 (SEQ ID NOs: 343-350), (xv) D94852_2E12 [MICA/B]_H2_D94852_2E12 [MICA/B]_L2 (SEQ ID NOs: 351-358), (xvi) D99136_2F7 [MICA/B]_H0_D99136_2F7 [MICA/B]_L0 (SEQ ID NOs: 359-366), (xvii) D99136_2F7 [MICA/B]_H1_D99136_2F7 [MICA/B]_L1 (SEQ ID NOs: 367-374), (xviii) D99136_2F7 [MICA/B]_H1_D99136_2F7 [MICA/B]_L2 (SEQ ID NOs: 375-382), (xix) D991362F7 [MICA/B]_H2_D99136_2F7 [MICA/B]_L1 (SEQ ID NOs: 383-390), (xx) D99136_2F7 [MICA/B]_H2_D99136_2F7 [MICA/B]_L2 (SEQ ID NOs: 391-398), (xxi) D103388_1C7 [MICA/B]_H0_D103388_1C7 [MICA/B]_L0 (SEQ ID NOs: 399-406), (xxii) D103388_1C7 [MICA/B]_H1_D103388_1C7 [MICA/B]_L1 (SEQ ID NOs: 407-414), (xxiii) D103388_1C7 [MICA/B]_H1_D103388_1C7 [MICA/B]_L2 (SEQ ID NOs: 415-422), (xxiv) D103388_1C7 [MICA/B]_H2_D103388_1C7 [MICA/B]_L1 (SEQ ID NOs: 423-430), (xxv) D103388_1C7 [MICA/B]_H2_D103388_1C7 [MICA/B]_L2 (SEQ ID NOs: 431-438), (xxvi) D103388_1D7 [MICA/B]_H0_D103388_1D7 [MICA/B]_L0 (SEQ ID NOs: 439-446), (xxvii) D103388_1D7 [MICA/B]_H1_D103388_1D7 [MICA/B]_L1 (SEQ ID NOs: 447-454), (xxviii) D103388_1D7 [MICA/B]_H1_D103388_1D7 [MICA/B]_L2 (SEQ ID NOs: 455-462), (xxix) D103388_1D7 [MICA/B]_H2_D103388_1D7 [MICA/B]_L1 (SEQ ID NOs: 463-470), (xxx) D103388_1D7 [MICA/B]_H2_D103388_1D7 [MICA/B]_L2 (SEQ ID NOs: 471-478), (xxxi) D105317_1A2 [MICA/B]_H0_D105317_1A2 [MICA/B]_L0 (SEQ ID NOs: 479-486), (xxxii) D105317_1A2 [MICA/B]_H1_D105317_1A2 [MICA/B]_L1 (SEQ ID NOs: 487-494), (xxxiii) D105317_1A2 [MICA/B]_H1_D105317_1A2 [MICA/B]_L2 (SEQ ID NOs: 495-502), (xxxiv) D105317_1A2 [MICA/B]_H2_D105317_1A2 [MICA/B]_L1 (SEQ ID NOs: 503-510), (xxxv) D105317_1A2 [MICA/B]_H2_D105317_1A2 [MICA/B]_L2 (SEQ ID NOs: 511-518), (xxxvi) D99136_2C11 [MICA/B]_H0_D99136_2C11 [MICA/B]_L0 (SEQ ID NOs: 519-526), (xxxvii) D99136_2C11 [MICA/B]_H1_D99136_2C11 [MICA/B]_L1 (SEQ ID NOs: 527-534), (xxxviii) D99136_2C11 [MICA/B]_H1_D99136_2C11 [MICA/B]_L2 (SEQ ID NOs: 535-542), (xxxix) D99136_2C11 [MICA/B]_H2_D99136_2C11 [MICA/B]_L1 (SEQ ID NOs: 543-550), (xl) D99136_2C11 [MICA/B]_H2_D99136_2C11 [MICA/B]_L2 (SEQ ID NOs: 551-558), (xli) D103388_1B11-2 [MICA/B]_H0_D103388_1B11-2 [MICA/B]_L0 (SEQ ID NOs: 559-566), (xlii) D105317_1B6 [MICA/B]_H0_D105317_1B6 [MICA/B]_L0 (SEQ ID NOs: 567-574), (xliii) D105317_1C8 [MICA/B]_H0_D105317_1C8 [MICA/B]_L0 (SEQ ID NOs: 575-582), (xliv) D105317_1F6 [MICA/B]_H0_D105317_1F6 [MICA/B]_L0 (SEQ ID NOs: 583-590), (xlv) D105317_1F7 [MICA/B]_H0_D105317_1F7 [MICA/B]_L0 (SEQ ID NOs: 591-598), (xlvi) D94837_1D3 [MICA/B]_H0_D94837_1D3 [MICA/B]_L0 (SEQ ID NOs: 599-606), (xlvii) D94837_1D8 [MICA/B]_H0_D94837_1D8 [MICA/B]_L0 (SEQ ID NOs: 607-614), (xlviii) D94837_1D9 [MICA/B]_H0_D94837_1D9 [MICA/B]_L0 (SEQ ID NOs: 615-622), (xlix) D94837_1E1 [MICA/B]_H0_D94837_1E1 [MICA/B]_L0 (SEQ ID NOs: 623-630), (1) D94852_2D4 [MICA/B]_H0_D94852_2D4 [MICA/B]_L0 (SEQ ID NOs: 631-638), (li) D94852_2G8 [MICA/B]_H0_D94852_2G8 [MICA/B]_L0 (SEQ ID NOs: 639-646), (lii) D88487_2A8 [MICA/B]_H0_D88487_2A8 [MICA/B]_L0 (SEQ ID NOs: 647-654), (liii) D94837_3B12 [MICA/B]_H0_D94837_3B12 [MICA/B]_L0 (SEQ ID NOs: 655-662), (liv) D99122_1H7 [MICA/B]_H0_D99122_1H7 [MICA/B]_L0 (SEQ ID NOs: 663-670), (lv) D99136_2E8 [MICA/B]_H0_D99136_2E8 [MICA/B]_L0 (SEQ ID NOs: 671-678), (lvi) D88487_2A8 [MICA/B]_H0_D88487_2A8 [MICA/B]_L0 (SEQ ID NOs: 647-654), (lvii) 3F9[MICA/B]_H0_3F9[MICA/B]_L0 (SEQ ID NOs: 679-686), (lviii) 6E1[MICA/B]_H0_6E1[MICA/B]_L0 (SEQ ID NOs: 687-694), (lix) 7C6[MICA/B]_H0_7C6[MICA/B]_L0 (SEQ ID NOs: 695-702), (lx) 13A9 [MICA/B]_H0_13A9 [MICA/B]_L0 (SEQ ID NOs: 703-710), and (lxi) 1D5 [MICA/B]_H0_1D5 [MICA/B]_L0 (SEQ ID NOs: 711-718) (see, e.g., FIGS. 21 and 22) attached as the scFv side of the backbones shown in FIGS. 12-15.

Exemplary anti-NKp46×anti-MICA/B 1+1 Fab×scFv format antibodies are depicted in FIGS. 31A and 31B. In some embodiments, such bispecific antibodies include, but are not limited to: XENP46810, XENP46811, and XENP46812, as are shown in FIGS. 31A and 31B. In some embodiments, the anti-NKp46×anti-MICA/B bispecific antibody is in a 1+1 Fab×scFv format, and the bispecific antibody comprises: (i) a first monomer, (ii) a second monomer, and (iii) a light chain. In an exemplary embodiment, the first monomer comprises SEQ ID NOs: 794, 797 and 800, the second monomer comprises SEQ ID NOs: 795, 798 and 801, and the light chain comprises SEQ ID NOs: 796, 799 and 802; see, e.g., FIG. 31.

### 2. 2+1 Fab×Fab-scFv Format

One heterodimeric scaffold that finds particular use in the antibodies described herein is the “2+1 Fab×Fab-scFv” format (also referred to herein as the “2+1 Fab₂-scFv-Fc” or “central-scFv” format), as is shown in FIG. 25C. In this embodiment, the format relies on the use of an inserted scFv domain thus forming a third antigen binding domain, wherein the Fab portions of the two monomers bind MICA/B and the “extra” scFv domain binds NKp46. Alternatively, the Fab portions of the two monomers can bind NKp46 and the “extra” scFv domain can bind MICA/B. The scFv domain is inserted between the Fc domain and the CH1-Fv region of one of the monomers, thus providing the third antigen binding domain. These MICA/B antibodies find use, for example, in the treatment of MICA/B-associated cancers.

In this embodiment, one monomer comprises a first heavy chain comprising a first variable heavy domain, a CH1 domain (and optional hinge) and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using optional domain linkers (VH1-CH1-[optional linker]-VH2-scFv linker-VL2-[optional linker]-CH2-CH3, or the opposite orientation for the scFv, VH1-CH1-[optional linker]-VL2-scFv linker-VH2-[optional linker]-CH2-CH3). The optional linkers can be any suitable peptide linkers, including, for example, the domain linkers included in FIG. 9 (see, e.g., SEQ ID NOs: 49-77). In some embodiments, the optional linker is a hinge or a fragment thereof. The other monomer is a standard Fab side (i.e., VH1-CH1-hinge-CH2-CH3). This embodiment further utilizes a common light chain comprising a variable light domain and a constant light domain, that associates with the heavy chains to form two identical Fabs that bind MICA/B (or, in other embodiments, to NKp46). As for many of the embodiments herein, these constructs include skew variants, pI variants, ablation variants, additional Fc variants, etc. as desired and described herein (see, e.g., FIGS. 4-7, 10, and 11).

In one embodiment, the 2+1 Fab×Fab-scFv format antibody includes an scFv with the V_{H} and V_{L} of a NKp46 binding domain sequence depicted in FIGS. 23 and 24, or a variant thereof. In one embodiment, the 2+1 Fab×Fab-scFv format includes two Fabs having the V_{H} and V_{L} of a MICA/B binding domain as depicted in FIGS. 21 and 22, or a variant thereof.

In exemplary embodiments, the MICA/B binding domain of the 2+1 Fab×Fab-scFv anti-NKp46×anti-MICA/B bispecific antibody includes the V_{H} and V_{L} NKp46 binding domain sequences finding particular use in these embodiments include, but are not limited to: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, or a variant thereof (see, e.g., FIGS. 23 and 24).

In particular embodiments, the αNKp46 V_{H} and V_{L} binding domain sequences are selected from the group including: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, or a variant thereof (see, e.g., FIGS. 23 and 24).

Any suitable MICA/B ABD can be included in the 2+1 Fab×Fab-scFv format antibody, included those provided herein. MICA/B ABDs that are of particular use in these embodiments include, but are not limited to, V_{H} and V_{L} domains selected from the group including: (i) SEQ ID Nos: 239-246 for D94837_E11_1 [MICA/B]_H0_D94837_1E11_1 [MICA/B]_L0, (ii) SEQ ID Nos: 247-254 for D94837_E11_1 [MICA/B]_H1_D94837_1E11_1 [MICA/B]_L1, (iii) SEQ ID Nos: 255-262 for D94837_1E11_1 [MICA/B]_H1_D94837_E11_1 [MICA/B]_L2, (iv) SEQ ID Nos: 263-270 for D94837_1E11_1 [MICA/B]_H2_D94837_E11_1 [MICA/B]_L1, (v) SEQ ID Nos: 271-278 for D94837_1E11_1 [MICA/B]_H2_D94837_E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0.

In particular embodiments, the αMICA/B V_{H}/V_{L} pairs are selected from the group including: (i) SEQ ID Nos: 239-246 for D94837_E11_1 [MICA/B]_H0_D94837_1E11_1 [MICA/B]_L0, (ii) SEQ ID Nos: 247-254 for D94837_E11_1 [MICA/B]_H1_D94837_1E11_1 [MICA/B]_L1, (iii) SEQ ID Nos: 255-262 for D94837_1E11_1 [MICA/B]_H1_D94837_E11_1 [MICA/B]_L2, (iv) SEQ ID Nos: 263-270 for D94837_1E11_1 [MICA/B]_H2_D94837_E11_1 [MICA/B]_L1, (v) SEQ ID Nos: 271-278 for D94837_1E11_1 [MICA/B]_H2_D94837_E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0.

In addition, the Fe domains of the 2+1 Fab×Fab-scFv format can comprise Fc ADCC variants (including, but not limited to, those shown in FIGS. 7 and 10), skew variants (e.g., a set of amino acid substitutions as shown in FIGS. 4, 10, and 11, with particularly useful skew variants being selected from the group including: (i) S364K/E357Q:L368D/K370S, (ii) L368D/K370S:S364K, (iii) L368E/K370S:S364K, (iv) T411T/E360E/Q362E:D401K, (v) L368D/K370S:S364K/E357L, (vi) K370S:S364K/E357Q, (vii) T366S/L368A/Y407V:T366W, and (viii) T366S/L368A/Y407V/Y349C:T366W/S354C), optionally ablation variants (including those shown in FIG. 6), optionally charged scFv linkers (including those shown in FIG. 8; see, e.g., SEQ ID NOs: 23-48), and the heavy chain comprises pI variants (including those shown in FIGS. 4, 5, and 10).

In some embodiments, the 2+1 Fab×Fab-scFv format antibody includes Fc ADCC variants (see, e.g., FIGS. 7 and 10), skew variants (see, e.g., FIGS. 4, 10, and 11), and/or pI variants (see, e.g., FIGS. 4, 5, and 10). Accordingly, some embodiments include 2+1 Fab×Fab-scFv formats that comprise: (i) a first monomer (the “Fab-scFv-Fc” monomer) that comprises the Fc ADCC variants S239D/I332E, the skew variants S364K/E357Q, and a variable heavy domain that, with the variable light domain of the common light chain, makes up an Fv that binds to MICA/B as outlined herein, and an scFv domain that binds to NKp46, (ii) a second monomer (the “Fab-Fc” monomer) that comprises the skew variants L368D/K370S, the pI variants N208D/Q295E/N384D/Q418E/N421D, and a variable heavy domain that, with variable light domain of the common light chain, makes up an Fv that binds to MICA/B as outlined herein, and (iii) a common light chain comprising the variable light domain and a constant light domain, where numbering is according to EU numbering. Further, bispecific antibodies in the 2+1 Fab×Fab-scFv format can also include one or more Fc domains with one or more ablation variants (see, e.g., FIG. 6). NKp46 binding domain sequences finding particular use in these embodiments include, but are not limited to: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, or variants thereof (see, e.g., FIGS. 23 and 24). MICA/B binding domain sequences finding particular use in these embodiments include, but are not limited to: (i) SEQ ID Nos: 239-246 for D94837_E11_1 [MICA/B]_H0_D94837_1E11_1 [MICA/B]_L0, (ii) SEQ ID Nos: 247-254 for D94837_E11_1 [MICA/B]_H1_D94837_1E11_1 [MICA/B]_L1, (iii) SEQ ID Nos: 255-262 for D94837_1E11_1 [MICA/B]_H1_D94837_E11_1 [MICA/B]_L2, (iv) SEQ ID Nos: 263-270 for D94837_1E11_1 [MICA/B]_H2_D94837_E11_1 [MICA/B]_L1, (v) SEQ ID Nos: 271-278 for D94837_1E11_1 [MICA/B]_H2_D94837_E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0, or variants thereof (see, e.g., FIGS. 21 and 22).

In some embodiments, the 2+1 Fab×Fab-scFv format antibody includes Fc ADCC variants (see, e.g., FIGS. 7 and 10), skew variants (see, e.g., FIGS. 4, 10, and 11), pI variants (see, e.g., FIGS. 4, 5, and 10), and/or FcRn variants (see, e.g., FIG. 10). Accordingly, some embodiments 2+1 Fab×Fab-scFv formats that comprise: (i) a first monomer (the “Fab-scFv-Fc” monomer) that comprises the Fc ADCC variants S239D/I332E, the skew variants S364K/E357Q, the FcRn variants M428L/N434S, and a variable heavy domain that, with the variable light domain of the common light chain, makes up an Fv that binds to MICA/B as outlined herein, and an scFv domain that binds to NKp46, (ii) a second monomer (the “Fab-Fc” monomer) that comprises the skew variants L368D/K370S, the pI variants N208D/Q295E/N384D/Q418E/N421D, the FcRn variants M428L/N434S, and a variable heavy domain that, with variable light domain of the common light chain, makes up an Fv that binds to MICA/B as outlined herein, and (iii) a common light chain comprising the variable light domain and a constant light domain, where numbering is according to EU numbering. Further, bispecific antibodies in the 2+1 Fab×Fab-scFv format can also include one or more Fc domains with one or more ablation variants (see, e.g., FIG. 6). NKp46 binding domain sequences finding particular use in these embodiments include, but are not limited to: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, or variants thereof (see, e.g., FIGS. 23 and 24). MICA/B binding domain sequences finding particular use in these embodiments include, but are not limited to: (i) SEQ ID Nos: 239-246 for D94837_E11_1 [MICA/B]_H0_D94837_1E11_1 [MICA/B]_L0, (ii) SEQ ID Nos: 247-254 for D94837_E11_1 [MICA/B]_H1_D94837_1E11_1 [MICA/B]_L1, (iii) SEQ ID Nos: 255-262 for D94837_1E11_1 [MICA/B]_H1_D94837_E11_1 [MICA/B]_L2, (iv) SEQ ID Nos: 263-270 for D94837_1E11_1 [MICA/B]_H2_D94837_E11_1 [MICA/B]_L1, (v) SEQ ID Nos: 271-278 for D94837_1E11_1 [MICA/B]_H2_D94837_E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0, or variants thereof (see, e.g., FIGS. 21 and 22).

FIGS. 12-15 show some exemplary Fc domain sequences that are useful with the 2+1 Fab×Fab-scFv format. The “monomer 1” sequences depicted in FIGS. 12-15 typically refer to the Fc domain of the “Fab-Fc heavy chain,” and the “monomer 2” sequences refer to the Fc domain of the “Fab-scFv-Fc heavy chain.”

In some embodiments, one or both of the Fc domains comprising the heterodimeric Fc backbone pairs are configured such that one or both of the Fc domains have wildtype FcγR effector function. In some embodiments, the heterodimeric Fc backbone pairs have wildtype FcγR effector function, including, but not limited to, those pairs of: SEQ ID NOs: 112-133, as shown in FIGS. 13A-13C.

In some embodiments, one or both of the Fc domains comprising the heterodimeric Fc backbone pairs include amino acid substitutions such that one or both of the Fc domains have increased ADCC activity. In certain embodiments, the amino acid substitutions include one or more of: S239D, 1332E, or S239D/I332E, or any of the Fc ADCC variants shown in FIGS. 7 and 10. In some instances, only monomer 1 or monomer 2 of the heterodimeric Fc backbone pairs include amino acid substitutions resulting in increased ADCC activity. In such an instance, the pair of heterodimeric Fc backbones may be referred to as an asymmetric, ADCC-enhanced heterodimeric Fc backbone pair. In other instances, both monomers (i.e., monomer 1 and monomer 2) of the heterodimeric Fc backbone pairs independently include one or more amino acid substitutions conferring an increased ADCC activity. In such an instance, the pair of heterodimeric Fc backbones may be referred to as a symmetric, ADCC-enhanced heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) includes amino acid variants conferring an increase in ADCC activity. In some instances, monomer 1 and monomer 2 can contain identical amino acid substitutions (e.g., both monomers contain a S239D substitution, an 1332E substitution, or both substitutions are present on both monomers). In other instances, monomer 1 and monomer 2 can contain at least one amino acid substitution that is the same (e.g., monomer 1 may contain the S239D/I332E substitutions, and monomer 2 contains the S239D substitution and is WT with respect to the 332 residue (or the reverse where monomer 2 is WT with respect to the 239 residue and contains the I332E substitution); monomer 2 may contain the S239D/I332E substitutions, and monomer 1 contains the S239D substitution and is WT with respect to the 332 residue (or the reverse where monomer 1 is WT with respect to the 239 residue and contains the 1332E substitution); etc.). In still other instances, monomer 1 and monomer 2 can contain amino acid substitutions that are unique to each monomer (e.g., S239D on monomer 1 and 1332E on monomer 2, or the reverse). In some embodiments, the heterodimeric Fc backbone pairs have increased ADCC activity, including, but not limited to, those pairs of: SEQ ID NOs: 134-163, as shown in FIGS. 14A-14C.

In some embodiments, one or both of the Fc domains comprising the heterodimeric Fc backbone pairs include amino acid substitutions such that one or both of the Fc domains have improved serum half-life. In certain embodiments, the amino acid substitutions include one or more of: M428L, N434S, N434A, M428L/N434S, M252Y/S254T/T256E, or M428L/N434A. In some instances, only monomer 1 or monomer 2 of the heterodimeric Fc backbone pairs include amino acid substitutions resulting in improved serum half-life. In such an instance, the pair of heterodimeric Fc backbones may be referred to as an asymmetric, Xtend heterodimeric Fc backbone pair. In other instances, both monomers (i.e., monomer 1 and monomer 2) of the heterodimeric Fc backbone pairs independently include one or more amino acid substitutions conferring an increase serum half-life. In such an instance, the pair of heterodimeric Fc backbones may be referred to as a symmetric, Xtend heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants conferring an increased serum half-life. In some instances, monomer 1 and monomer 2 can contain identical amino acid substitutions. In other instances, monomer 1 and monomer 2 can contain at least one amino acid substitution that is the same (e.g., both monomers contain the M428L substitution, one monomer further contains the N434S substitution, and the other monomer contains the N434A mutation). In some instances, monomer 1 and monomer 2 can contain amino acid substitutions that are unique to each monomer (e.g., N434S on one monomer, and N434A on the other monomer).

In some embodiments, one or both of the Fc domains comprising the heterodimeric Fc backbone pairs include amino acid substitutions such that one or both of the Fc domains have increased ADCC activity and improved serum half-life. In certain embodiments, the amino acid substitutions include: (i) one or more of amino acid substitutions selected from the group including: S239D, 1332E, and S239D/I332E, or any of the Fc ADCC variants shown in FIGS. 7 and 10, and (ii) one or more amino acid substitutions selected from the group including: M428L, N434S, N434A, M428L/N434S, M252Y/S254T/T256E, and M428L/N434A. In some instances, only monomer 1 or monomer 2 of the heterodimeric Fc backbone pairs include amino acid substitutions resulting in increased ADCC and increased serum half-life. In such an instance, the pair of heterodimeric Fc backbones may be referred to as an asymmetric, ADCC-enhanced heterodimeric Fc backbone pair with Xtend. In other instances, both monomers (i.e., monomer 1 and monomer 2) of the heterodimeric Fc backbone pairs independently comprise amino acid substitutions resulting in increased ADCC and increased serum half-life. In such an instance, the pair of heterodimeric Fc backbones may be referred to as a symmetric, ADCC-enhanced heterodimeric Fc backbone pair with Xtend. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants conferring an increase in ADCC activity and improved serum half-life. In some instances, monomer 1 and monomer 2 can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased ADCC activity and increased serum-half-life, including, but not limited to, those pairs of: SEQ ID NOs: 164-193, as shown in FIGS. 15A-15C.

In some embodiments, one or both of the Fe domains comprising the heterodimeric Fc backbone pairs include amino acid substitutions such that one or both of the Fc domains have an ablated FcγR function. Nonlimiting examples of amino acid substitutions (and combinations thereof) conferring an ablated FcγR function are shown in, for example, FIG. 6. In some instances, only monomer 1 or monomer 2 of the heterodimeric Fc backbone pairs include amino acid substitutions that result in an ablated FcγR function (see, e.g., FIG. 6). In such an instance, the pair of heterodimeric Fc backbones may be referred to as an asymmetric, FcKO heterodimeric Fc backbone pair. In other instances, both monomers (i.e., monomer 1 and monomer 2) of the heterodimeric Fc backbone pairs independently comprise one or more amino acid substitutions resulting in an ablated FcγR function (see, e.g., FIG. 6). In such an instance, the pair of heterodimeric Fc backbones may be referred to as a symmetric, FcKO heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants resulting in an ablated FcγR function. In some embodiments, the heterodimeric Fc backbone pairs include amino acid substitutions such that the Fc domains have an ablated FcγR effector function, including, but not limited to, those pairs of: SEQ ID NOs: 78-111, as shown in FIGS. 12A-12E.

Further, FIG. 19 provides useful C_{L} sequences that can be used with this format.

Exemplary anti-NKp46×anti-MICA/B 2+1 Fab×Fab-scFv format antibodies are depicted in FIG. 33. In some embodiments, such bispecific antibodies include, but are not limited to: XENP47438 and XENP47439, as are shown in FIG. 33. In some embodiments, the anti-NKp46×anti-MICA/B bispecific antibody is in a 2+1 Fab×Fab-scFv format, and the bispecific antibody comprises: (i) a first monomer, (ii) a second monomer, and (iii) a light chain. In an exemplary embodiment, the first monomer comprises SEQ ID NOs: 809 and 812, the second monomer comprises SEQ ID NOs: 810 and 813, and the light chain comprises SEQ ID NOs: 811 and 814; see, e.g., FIG. 33.

### 3. 2+1 mAb-scFv Format

One heterodimeric scaffold that finds particular use in the antibodies described herein is the “2+1 mAb-scFv” format (also referred to herein as the “mAb-scFv” format), as is shown in FIG. 25E. In this embodiment, the format relies on the use of a C-terminal attachment of a scFv to one of the monomers, thus forming a third antigen binding domain, wherein the Fab portions of the two monomers bind MICA/B and the “extra” scFv domain binds NKp46. Alternatively, the Fab portions of the two monomers can bind NKp46 and the “extra” scFv domain can bind MICA/B. Thus, the first monomer comprises a first heavy chain (comprising a variable heavy domain and a constant domain), with a C-terminally covalently attached scFv comprising a scFv variable light domain, an scFv linker and a scFv variable heavy domain in either orientation (VH1-CH1-hinge-CH2-CH3-[optional linker]-VH2-scFv linker-VL2 or VH1-CH1-hinge-CH2-CH3-[optional linker]-VL2-scFv linker-VH2). This embodiment further utilizes a common light chain comprising a variable light domain and a constant light domain, that associates with the heavy chains to form two identical Fabs that bind MICA/B (or, in other embodiments, to NKp46). In some embodiments, an exemplary 2+1 mAb-scFv format includes: (i) a first Fc comprising an N-terminal Fab arm that binds MICA/B, (ii) a second Fc comprising an N-terminal Fab arm that binds MICA/B, and (iii) a C-terminal scFv that binds NKp46; or the reverse, wherein the Fab arms bind NKp46 and the C-terminal scFv binds MICA/B. Such a format can include a first monomer comprising, from the N-terminus to the C-terminus, VH1-CH1-hinge-CH2-CH3, a second monomer comprising, from the N-terminus to the C-terminus, VH1-CH1-hinge-CH2-CH3-scFv, and a third monomer comprising, from the N-terminus to the C-terminus, V_{L}-C_{L}, wherein the first VH1-V_{L} pair bind MICA/B, the second VH1-V_{L} pair bind MICA/B, and the scFv binds NKp46. In another embodiment of the 2+1 mAb-scFv format, the first and second VH1-V_{L} pairs bind NKp46 and the scFv binds MICA/B. As for many of the embodiments herein, these constructs include skew variants, pI variants, ablation variants, additional Fc variants, etc. as desired and described herein.

The antibodies described herein provide 2+1 mAb-scFv formats, where the NKp46 domain sequences comprise variable heavy and variable light domains selected from the group including: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, and variants thereof (see, e.g., FIGS. 23 and 24); and the MICA/B binding domain sequences comprise variable heavy and variable light domains selected from the group including: (i) SEQ ID Nos: 239-246 for D94837_1E11_1 [MICA/B]_H0_D94837_E11_1 [MICA/B]_L0, (ii) SEQ ID Nos: 247-254 for D94837_1E11_1 [MICA/B]_H1_D94837_1E11_1 [MICA/B]_L1, (iii) SEQ ID Nos: 255-262 for D94837_1E11_1 [MICA/B]_H1_D94837_E11_1 [MICA/B]_L2, (iv) SEQ ID Nos: 263-270 for D94837_1E11_1 [MICA/B]_H2_D94837_1E11_1 [MICA/B]_L1, (v) SEQ ID Nos: 271-278 for D94837_1E11_1 [MICA/B]_H2_D94837_E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0, and variants thereof (see, e.g., FIGS. 21 and 22).

In particular embodiments, the αNKp46 V_{H} and V_{L} binding domain sequences are selected from the group including: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, or a variant thereof (see, e.g., FIGS. 23 and 24).

In particular embodiments, the αMICA/B V_{H}/V_{L} pairs are selected from the group including: (i) SEQ ID Nos: 239-246 for D94837_E11_1 [MICA/B]_H0_D94837_1E11_1 [MICA/B]_L0, (ii) SEQ ID Nos: 247-254 for D94837_E11_1 [MICA/B]_H1_D94837_1E11_1 [MICA/B]_L1, (iii) SEQ ID Nos: 255-262 for D94837_1E11_1 [MICA/B]_H1_D94837_E11_1 [MICA/B]_L2, (iv) SEQ ID Nos: 263-270 for D94837_1E11_1 [MICA/B]_H2_D94837_E11_1 [MICA/B]_L1, (v) SEQ ID Nos: 271-278 for D94837_1E11_1 [MICA/B]_H2_D94837_E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0, or a variant thereof (see, e.g., FIGS. 21 and 22).

In some embodiments, the 2+1 mAb-scFv format includes one or more of: (a) Fc ADCC variants (see, e.g., FIGS. 7 and 10), (b) skew variants (see, e.g., FIGS. 4, 10, and 11), and/or pI variants (see, e.g., FIGS. 4, 5, and 10). Accordingly, some embodiments include mAb-scFv formats that comprise: (i) a first monomer that comprises the Fc ADCC variants S239D/I332E, the skew variants S364K/E357Q, and a variable heavy domain that, with the variable light domain of the common light chain, makes up an Fv that binds to MICA/B as outlined herein, and a scFv domain that binds to NKp46, (ii) a second monomer that comprises the skew variants L368D/K370S, the pI variants N208D/Q295E/N384D/Q418E/N421D, and a variable heavy domain that, with the variable light domain of the common light chain, makes up an Fv that binds to MICA/B as outlined herein, and (iii) a common light chain comprising a variable light domain and a constant light domain. Further, bispecific antibodies in the 2+1 mAb-scFv format can also include one or more Fc domains with one or more ablation variants (see, e.g., FIG. 6). NKp46 binding domain sequences finding particular use in these embodiments include, but are not limited to: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, or variants thereof (see, e.g., FIGS. 23 and 24). MICA/B binding domain sequences finding particular use in these embodiments include, but are not limited to: (i) SEQ ID Nos: 239-246 for D94837_E11_1 [MICA/B]_H0_D94837_1E11_1 [MICA/B]_L0, (ii) SEQ ID Nos: 247-254 for D94837_E11_1 [MICA/B]_H1_D94837_1E11_1 [MICA/B]_L1, (iii) SEQ ID Nos: 255-262 for D94837_1E11_1 [MICA/B]_H1_D94837_E11_1 [MICA/B]_L2, (iv) SEQ ID Nos: 263-270 for D94837_1E11_1 [MICA/B]_H2_D94837_E11_1 [MICA/B]_L1, (v) SEQ ID Nos: 271-278 for D94837_1E11_1 [MICA/B]_H2_D94837_E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0, or variants thereof (see, e.g., FIGS. 21 and 22).

In some embodiments, the 2+1 mAb-scFv format includes Fc ADCC variants (see, e.g., FIGS. 7 and 10), skew variants (see, e.g., FIGS. 4, 10, and 11), pI variants (see, e.g., FIGS. 4, 5, and 10), and/or FcRn variants (see, e.g., FIG. 10). Accordingly, some embodiments include mAb-scFv formats that comprise: (i) a first monomer that comprises the Fc ADCC variants S239D/I332E, the skew variants S364K/E357Q, the FcRn variants M428L/N434S, and a variable heavy domain that, with the variable light domain of the common light chain, makes up an Fv that binds to MICA/B as outlined herein, and a scFv domain that binds to NKp46, (ii) a second monomer that comprises the skew variants L368D/K370S, the pI variants N208D/Q295E/N384D/Q418E/N421D, the FcRn variants M428L/N434S, and a variable heavy domain that, with the variable light domain of the common light chain, makes up an Fv that binds to MICA/B as outlined herein, and (iii) a common light chain comprising a variable light domain and a constant light domain. Further, bispecific antibodies in the mAb-scFv format can also include one or more Fc domains with one or more ablation variants (see, e.g., FIG. 6). NKp46 binding domain sequences finding particular use in these embodiments include, but are not limited to: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, or variants thereof (see, e.g., FIGS. 23 and 24). MICA/B binding domain sequences finding particular use in these embodiments include, but are not limited to: (i) SEQ ID Nos: 239-246 for D94837_E11_1 [MICA/B]_H0_D94837_1E11_1 [MICA/B]_L0, (ii) SEQ ID Nos: 247-254 for D94837_E11_1 [MICA/B]_H1_D94837_1E11_1 [MICA/B]_L1, (iii) SEQ ID Nos: 255-262 for D94837_1E11_1 [MICA/B]_H1_D94837_E11_1 [MICA/B]_L2, (iv) SEQ ID Nos: 263-270 for D94837_1E11_1 [MICA/B]_H2_D94837_E11_1 [MICA/B]_L1, (v) SEQ ID Nos: 271-278 for D94837_1E11_1 [MICA/B]_H2_D94837_E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0, or variants thereof (see, e.g., FIGS. 21 and 22).

FIGS. 16A-16G show some exemplary Fc domain sequences that are useful with the 2+1 mAb-scFv format. The “monomer 1” sequences depicted in FIGS. FIGS. 16A-16G typically refer to the Fc domain of the “Fab-Fc heavy chain,” and the “monomer 2” sequences refer to the Fc domain of the “Fab-Fc-scFv heavy chain.”

In some embodiments, one or both of the Fc domains comprising the heterodimeric Fc backbone pairs are configured such that one or both of the Fc domains have wildtype FcγR effector function. In some embodiments, the heterodimeric Fc backbone pairs have wildtype FcγR effector function, including, but not limited to, those pairs of: SEQ ID NOs: 134, 136, 194-199, as shown in FIG. 16A.

In some embodiments, one or both of the Fc domains comprising the heterodimeric Fc backbone pairs include amino acid substitutions such that one or both of the Fc domains have increased ADCC activity. In certain embodiments, the amino acid substitutions include one or more of: S239D, 1332E, or S239D/I332E, or any of the Fc ADCC variants shown in FIGS. 7 and 10. In some instances, only monomer 1 or monomer 2 of the heterodimeric Fc backbone pairs include amino acid substitutions resulting in increased ADCC activity. In such an instance, the pair of heterodimeric Fc backbones may be referred to as an asymmetric, ADCC-enhanced heterodimeric Fc backbone pair. In other instances, both monomers (i.e., monomer 1 and monomer 2) of the heterodimeric Fc backbone pairs independently include one or more amino acid substitutions conferring an increased ADCC activity. In such an instance, the pair of heterodimeric Fc backbones may be referred to as a symmetric, ADCC-enhanced heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fe domains (in the case of symmetric pairs) includes amino acid variants conferring an increase in ADCC activity. In some instances, monomer 1 and monomer 2 can contain identical amino acid substitutions (e.g., both monomers contain a S239D substitution, an 1332E substitution, or both substitutions are present on both monomers). In other instances, monomer 1 and monomer 2 can contain at least one amino acid substitution that is the same (e.g., monomer 1 may contain the S239D/I332E substitutions, and monomer 2 contains the S239D substitution and is WT with respect to the 332 residue (or the reverse where monomer 2 is WT with respect to the 239 residue and contains the I332E substitution); monomer 2 may contain the S239D/I332E substitutions, and monomer 1 contains the S239D substitution and is WT with respect to the 332 residue (or the reverse where monomer 1 is WT with respect to the 239 residue and contains the I332E substitution); etc.). In still other instances, monomer 1 and monomer 2 can contain amino acid substitutions that are unique to each monomer (e.g., S239D on monomer 1 and 1332E on monomer 2, or the reverse). In some embodiments, the heterodimeric Fc backbone pairs have increased ADCC activity, including, but not limited to, those pairs of: SEQ ID NOs: 134, 136, 138, 140, 142, 144, 146, 148, 150, 152, 154, 156, 158, 160, 162, 164, 166, and 194-214, as shown in FIGS. 16A-16D.

In some embodiments, one or both of the Fc domains comprising the heterodimeric Fc backbone pairs include amino acid substitutions such that one or both of the Fc domains have improved serum half-life. In certain embodiments, the amino acid substitutions include one or more of: M428L, N434S, N434A, M428L/N434S, M252Y/S254T/T256E, or M428L/N434A. In some instances, only monomer 1 or monomer 2 of the heterodimeric Fc backbone pairs include amino acid substitutions resulting in improved serum half-life. In such an instance, the pair of heterodimeric Fc backbones may be referred to as an asymmetric, Xtend heterodimeric Fc backbone pair. In other instances, both monomers (i.e., monomer 1 and monomer 2) of the heterodimeric Fc backbone pairs independently include one or more amino acid substitutions conferring an increase serum half-life. In such an instance, the pair of heterodimeric Fc backbones may be referred to as a symmetric, Xtend heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants conferring an increased serum half-life. In some instances, monomer 1 and monomer 2 can contain identical amino acid substitutions. In other instances, monomer 1 and monomer 2 can contain at least one amino acid substitution that is the same (e.g., both monomers contain the M428L substitution, one monomer further contains the N434S substitution, and the other monomer contains the N434A mutation). In some instances, monomer 1 and monomer 2 can contain amino acid substitutions that are unique to each monomer (e.g., N434S on one monomer, and N434A on the other monomer).

In some embodiments, one or both of the Fc domains comprising the heterodimeric Fc backbone pairs include amino acid substitutions such that one or both of the Fc domains have increased ADCC activity and improved serum half-life. In certain embodiments, the amino acid substitutions include: (i) one or more of amino acid substitutions selected from the group including: S239D, 1332E, and S239D/I332E, or any of the Fc ADCC variants shown in FIGS. 7 and 10, and (ii) one or more amino acid substitutions selected from the group including: M428L, N434S, N434A, M428L/N434S, M252Y/S254T/T256E, and M428L/N434A. In some instances, only monomer 1 or monomer 2 of the heterodimeric Fc backbone pairs include amino acid substitutions resulting in increased ADCC and increased serum half-life. In such an instance, the pair of heterodimeric Fc backbones may be referred to as an asymmetric, ADCC-enhanced heterodimeric Fc backbone pair with Xtend. In other instances, both monomers (i.e., monomer 1 and monomer 2) of the heterodimeric Fc backbone pairs independently comprise amino acid substitutions resulting in increased ADCC and increased serum half-life. In such an instance, the pair of heterodimeric Fc backbones may be referred to as a symmetric, ADCC-enhanced heterodimeric Fc backbone pair with Xtend. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants conferring an increase in ADCC activity and improved serum half-life. In some instances, monomer 1 and monomer 2 can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased ADCC activity and increased serum-half-life, including, but not limited to, those pairs of: SEQ ID NOs: 158, 160, 162, 164, 166, 168, 170, 172, 174, 176, 178, 180, 182, 184, 186, 188, 190, 192, and 210-227, as shown in FIGS. 16D-16G.

In some embodiments, one or both of the Fe domains comprising the heterodimeric Fc backbone pairs include amino acid substitutions such that one or both of the Fc domains have an ablated FcγR function. Nonlimiting examples of amino acid substitutions (and combinations thereof) conferring an ablated FcγR function are shown in, for example, FIG. 6. In some instances, only monomer 1 or monomer 2 of the heterodimeric Fc backbone pairs include amino acid substitutions that result in an ablated FcγR function (see, e.g., FIG. 6). In such an instance, the pair of heterodimeric Fc backbones may be referred to as an asymmetric, FcKO heterodimeric Fc backbone pair. In other instances, both monomers (i.e., monomer 1 and monomer 2) of the heterodimeric Fc backbone pairs independently comprise one or more amino acid substitutions resulting in an ablated FcγR function (see, e.g., FIG. 6). In such an instance, the pair of heterodimeric Fc backbones may be referred to as a symmetric, FcKO heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants resulting in an ablated FcγR function. In some embodiments, the heterodimeric Fc backbone pairs include amino acid substitutions such that the Fc domains have an ablated FcγR effector function, including, but not limited to, those pairs of: SEQ ID NOs: 134, 136, 194-199, as shown in FIG. 16A.

Exemplary anti-NKp46×anti-MICA/B 2+1 mAb-scFv format antibodies are depicted in FIG. 35. In some embodiments, such bispecific antibodies include, but are not limited to: XENP47446 and XENP47447, as are shown in FIG. 35. In some embodiments, the anti-NKp46×anti-MICA/B bispecific antibody is in a 2+1 mAb-scFv format, and the bispecific antibody comprises: (i) a first monomer, (ii) a second monomer, and (iii) a light chain. In an exemplary embodiment, the first monomer comprises SEQ ID NOs: 821 and 824, the second monomer comprises SEQ ID NOs: 822 and 825, and the light chain comprises SEQ ID NOs: 823 and 826; see, e.g., FIG. 35.

### 4. 1+1 Empty×Fab-scFv Format

One heterodimeric scaffold that finds particular use in the antibodies described herein is the “1+1 Empty×Fab-scFv” format (also referred to herein as the “one-armed central-scFv” format), as is shown in FIG. 25B. In this embodiment, one monomer comprises just an Fc domain (i.e., a first Fc domain), while the other monomer includes a Fab domain (a first ABD), a scFv (a second ABD), and an Fc domain (i.e., a second Fc domain), where the scFv domain is inserted between the first Fc domain and the second Fc domain. In this format, the Fab portion binds one receptor target and the scFv binds another. In this format, either the Fab portion binds a MICA/B antigen and the scFv binds a NKp46 antigen, or vice versa.

In this embodiment, one monomer comprises a first heavy chain comprising a first variable heavy domain, a CH1 domain, and a Fc domain, with a scFv comprising a scFv variable light domain, an scFv linker, and a scFv variable heavy domain. The scFv is covalently attached between the C-terminus of the CH1 domain of the heavy constant domain and the N-terminus of the first Fc domain using domain linkers, in either orientation, VH1-CH1-[optional domain linker]-VH2-scFv linker-VL2-[optional domain linker]-CH2-CH3 or VH1-CH1-[optional domain linker]-VL2-scFv linker-VH2-[optional domain linker]-CH2-CH3. The second monomer comprises an Fc domain (CH2-CH3). This embodiment further utilizes a light chain comprising a variable light domain and a constant light domain that associates with the heavy chain to form a Fab. As for many of the embodiments herein, these constructs can include one or more of: (i) Fc ADCC variants, (ii) pI variants, (iii) ablation variants, (iv) skew variants, (v) variants that improve serum half-life, as well as any combination thereof, as desired and described herein.

Any suitable NKp46 antigen binding domain (as described herein and in the Figures, or a variant thereof) can be included in the subject one-armed central-scFv format antibody.

Any suitable MICA/B antigen binding domain (as described herein and in the Figures, or a variant thereof) can be included in the subject one-armed central-scFv format antibody.

In addition, the Fc domains of the 1+1 Empty×Fab-scFv format can comprise Fc ADCC variants (including, but not limited to, those shown in FIGS. 7 and 10), skew variants (e.g., a set of amino acid substitutions as shown in FIGS. 4, 10, and 11, with particularly useful skew variants being selected from the group including: (i) S364K/E357Q:L368D/K370S, (ii) L368D/K370S:S364K, (iii) L368E/K370S:S364K, (iv) T411T/E360E/Q362E:D401K, (v) L368D/K370S:S364K/E357L, (vi) K370S:S364K/E357Q, (vii) T366S/L368A/Y407V:T366W, and (viii) T366S/L368A/Y407V/Y349C:T366W/S354C), optionally ablation variants (including those shown in FIG. 6), optionally charged scFv linkers (including those shown in FIG. 8; see, e.g., SEQ ID NOs: 23-48), and the heavy chain can comprise pI variants (including those shown in FIGS. 4, 5, and 10).

Exemplary anti-NKp46×anti-MICA/B 1+1 Empty×Fab-scFv format antibodies are depicted in FIG. 32. In some embodiments, such bispecific antibodies include, but are not limited to: XENP47274 and XENP47281, as are shown in FIG. 32. In some embodiments, the anti-NKp46×anti-MICA/B bispecific antibody is in a 1+1 Empty×Fab-scFv format, and the bispecific antibody comprises: (i) a first monomer, (ii) a second monomer, and (iii) a light chain. In an exemplary embodiment, the first monomer comprises SEQ ID NOs: 803 and 806, the second monomer comprises SEQ ID NOs: 804 and 807, and the light chain comprises SEQ ID NOs: 805 and 808; see, e.g., FIG. 32.

### 5. 2+1 Fab2×scFv Format

One heterodimeric scaffold that finds particular use in the antibodies described herein is the “2+1 Fab2×scFv” format (also referred to herein as the “2+1 stack Fab₂-scFv-Fc” or “stack Fab₂-scFv-Fc” format), as is shown in FIG. 25D. In this embodiment, the format relies on the use of a “stacked” Fab portion that binds a first target antigen (e.g., a MICA/B antigen), and an scFv domain that binds a second target antigen (e.g., a NKp46 antigen); or the reverse, wherein the first target antigen is a NKp46 antigen, and the second target antigen is a MICA/B antigen. In this format, the first monomer comprises, from N-terminus to C-terminus, a first heavy chain (comprising a variable heavy domain and a constant domain), a domain linker, and a second heavy chain (comprising a variable heavy domain and a constant domain), and a first Fc domain; the second monomer comprises, from N-terminus to C-terminus, a scFv comprising a scFv variable light domain, an scFv linker, and a scFv variable heavy domain in either orientation ((VH1-CH1-hinge-CH2-CH3-[optional linker]-VH2-scFv linker-VL2) or (VH1-CH1-hinge-CH2-CH3-[optional linker]-VL2-scFv linker-VH2)), and a second Fc domain; and the third monomer comprises a common light chain including a variable light domain and a constant light domain, that associates with the heavy chains to form two identical Fabs that bind a MICA/B antigen (or, in other embodiments, a NKp46 antigen). As for many of the embodiments herein, these constructs can include one or more of: (i) Fc ADCC variants, (ii) pI variants, (iii) ablation variants, (iv) skew variants, (v) variants that improve serum half-life, as well as any combination thereof, as desired and described herein.

Any suitable NKp46 antigen binding domain (as described herein and in the Figures, or a variant thereof) can be included in the subject 2+1 Fab2×scFv format antibody.

Any suitable MICA/B antigen binding domain (as described herein and in the Figures, or a variant thereof) can be included in the subject 2+1 Fab2×scFv format antibody.

In addition, the Fc domains of the 2+1 Fab2×scFv format can comprise Fc ADCC variants (including, but not limited to, those shown in FIGS. 7 and 10), skew variants (e.g., a set of amino acid substitutions as shown in FIGS. 4, 10, and 11, with particularly useful skew variants being selected from the group including: (i) S364K/E357Q:L368D/K370S, (ii) L368D/K370S:S364K, (iii) L368E/K370S:S364K, (iv) T411T/E360E/Q362E:D401K, (v) L368D/K370S:S364K/E357L, (vi) K370S:S364K/E357Q, (vii) T366S/L368A/Y407V:T366W, and (viii) T366S/L368A/Y407V/Y349C:T366W/S354C), optionally ablation variants (including those shown in FIG. 6), optionally charged scFv linkers (including those shown in FIG. 8; see, e.g., SEQ ID NOs: 23-48), and the heavy chain can comprise pI variants (including those shown in FIGS. 4, 5, and 10).

Exemplary anti-NKp46×anti-MICA/B 2+1 Fab2×scFv format antibodies are depicted in FIG. 34. In some embodiments, such bispecific antibodies include, but are not limited to: XENP47442 and XENP47443, as are shown in FIG. 34. In some embodiments, the anti-NKp46×anti-MICA/B bispecific antibody is in a 2+1 Fab2×scFv format, and the bispecific antibody comprises: (i) a first monomer, (ii) a second monomer, and (iii) a light chain. In an exemplary embodiment, the first monomer comprises SEQ ID NOs: 815 and 818, the second monomer comprises SEQ ID NOs: 816 and 819, and the light chain comprises SEQ ID NOs: 817 and 820; see, e.g., FIG. 34.

### 6. 1+1 CLC Format

One heterodimeric scaffold that finds particular use in the antibodies described herein is the “1+1 common light chain” (or “1+1 CLC”) format. The 1+1 CLC format antibody includes: a first monomer that includes a VH1-CH1-hinge-CH2-CH3, wherein VH1 is a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer “common light chain” comprising V_{L}—C_{L}, wherein V_{L} is a common variable light domain and C_{L} is a constant light domain. In such embodiments, the V_{L} pairs with the VH1 to form a first binding domain with a first antigen binding specificity; and the V_{L} pairs with the VH2 to form a second binding domain with a second antigen binding specificity. In some embodiments, the 1+1 CLC format antibody is a bivalent antibody.

In some embodiments, the first and/or second Fc domains of the 1+1 CLC format are variant Fc domains. Generally, variant Fc domains can comprise one or more of: (a) one or more Fc ADCC variants (see, e.g., FIGS. 7 and 10), (b) one or more skew variants (see, e.g., FIGS. 4, 10, and 11), (c) one or more ablation variants (see, e.g., FIG. 6), (d) one or more FcRn variants (e.g., M428L, N434S, N434A, M428L/N434S, M428L/N434A, etc.), (e) a scFv linker (including those shown in FIG. 8; see, e.g., SEQ ID NOs: 23-48), and one or both of the heavy chains can comprise one or more pI variants (see, e.g., FIGS. 4, 5, and 10). The various types of variants can be used alone, or in combination with other variant types described herein.

In some embodiments, one or both of the Fc domains are configured such that one or both of the Fc domains have wildtype FcγR effector function. In some embodiments, the heterodimeric Fc backbone pairs have wildtype FcγR effector function, including, but not limited to, those pairs of Fc backbones shown in the Figures.

In some embodiments, one or both of the Fc domains include one or more amino acid substitutions such that one or both of the Fc domains have increased ADCC activity. In certain embodiments, the amino acid substitutions include one or more of: S239D, I332E, or S239D/I332E, or any of the Fc ADCC variants shown in FIGS. 7 and 10. In some instances, the Fc backbone pair comprises an asymmetric, ADCC-enhanced heterodimeric Fc backbone pair (as described above). In other embodiments, the Fc backbone pair comprises a symmetric, ADCC-enhanced heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased ADCC activity, including, but not limited to, those pairs of Fc backbones shown in the Figures. In some embodiments, one or both of the Fc domains include one or more amino acid substitutions such that one or both of the Fc domains have increased ADCC activity, and optionally one or more of: (i) one or more skew variants, (ii) one or more ablation variants, (iii) one or more variants that improve serum half-life, (iv) one or more pI variants in the constant domain of the first and/or second monomer, or (v) any combination thereof.

In some embodiments, one or both of the Fc domains include heterodimerization skew variants (e.g., a set of amino acid substitutions, including, but not limited to, those shown in FIGS. 4, 10, and 11). Particularly useful heterodimerization skew variants include, but are not limited to: (i) S364K/E357Q: L368D/K370S, (ii) L368D/K370S: S364K, (iii) L368E/K370S: S364K, (iv) T411T/K360E/Q362E: D401K, (v) L368D/K370S: S364K/E357L, (vi) K370S: S364K/E357Q, (vii) T366S/L368A/Y407V: T366W, and (viii) T366S/L368A/Y407V/Y349C: T366W/S354C (according to EU numbering). In exemplary embodiments, one of the first or second variant Fc domains includes the heterodimerization skew variants L368D/K370S, and the other of the second or first variant Fc domain includes heterodimerization skew variants S364K/E357Q, wherein numbering is according to EU numbering. In some instances, the heterodimerization skew variants are only included on one of the two Fc domains. In such instances, the heterodimerization skew variants are in an asymmetric configuration. In some other instances, the heterodimerization skew variants are included on both of the Fc domains. In such instances, the heterodimerization skew variants are in a symmetric configuration. As used herein, the terms “asymmetric configuration” and “symmetric configuration” are not intended to mean that the heterodimeric Fc backbone pairs comprise identical heterodimerization skew variants, but rather that only one Fc domain (in the case of asymmetric configurations) or both Fc domains (in the case of symmetric configurations) includes heterodimerization skew variants. In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, one or both of the Fe domains include heterodimerization skew variants, and optionally one or more of: (i) one or more Fc ADCC variants, (ii) one or more ablation variants, (iii) one or more variants that improve serum half-life, (iv) one or more pI variants in the constant domain of the first and/or second monomer, or (v) any combination thereof.

In some embodiments, one or both of the Fc domains include one or more amino acid substitutions such that one or both of the Fc domains have improved serum half-life. In certain embodiments, the amino acid substitutions include one or more of: M428L, N434S, N434A, M428L/N434S, M252Y/S254T/T256E, or M428L/N434A. In some instances, the Fc backbone pair comprises an asymmetric, Xtend heterodimeric Fc backbone pair (as described above). In other embodiments, the Fc backbone pair comprises a symmetric, Xtend heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased serum half-life, including, but not limited to, those pairs of Fc backbones shown in the Figures. In some embodiments, one or both of the Fc domains include one or more amino acid substitutions such that one or both of the Fc domains have improved serum half-life, and optionally one or more of: (i) one or more skew variants, (ii) one or more ablation variants, (iii) one or more Fc ADCC variants, (iv) one or more pI variants in the constant domain of the first and/or second monomer, or (v) any combination thereof.

In some embodiments, one or both of the Fc domains include one or more amino acid substitutions such that one or both of the Fc domains have an ablated FcγR function. Nonlimiting examples of amino acid substitutions (and combinations thereof) conferring an ablated FcγR function are shown in, for example, FIG. 6. In some instances, the Fc backbone pair comprises an asymmetric FcKO heterodimeric Fc backbone pair (as described above). In other embodiments, the Fc backbone pair comprises a symmetric, FcKO heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have ablated FcγR function, including, but not limited to, those pairs of Fc backbones shown in the Figures. In some embodiments, each of the first and second Fc domains include the ablation variants E233P/L234V/L235A/G236_/S267K, wherein numbering is according to EU numbering. In some embodiments, one or both of the Fc domains include one or more amino acid substitutions such that one or both of the Fc domains have an ablated FcγR function, and optionally one or more of: (i) one or more skew variants, (ii) one or more Fc ADCC variants, (iii) one or more variants that improve serum half-life, (iv) one or more pI variants in the constant domain of the first and/or second monomer, or (v) any combination thereof.

In some embodiments, the constant domain (CH1-hinge-CH2-CH3) of the first and/or second monomer includes pI variants (including, but not limited to, those shown in FIGS. 4, 5, and 10). In exemplary embodiments, the constant domain (CH1-hinge-CH2-CH3) of the first or second monomer includes pI variants N208D/Q295E/N384D/Q418E/N421D, wherein numbering is according to EU numbering. In some embodiments, the constant domain of the first and/or second monomer includes pI variants (including, but not limited to, those shown in FIGS. 4, 5, and 10), and the first and/or second Fc domain optionally contains one or more of: (i) one or more skew variants, (ii) one or more ablation variants, (iii) one or more variants that improve serum half-life, (iv) one or more Fc ADCC variants, or (v) any combination thereof.

In some embodiments, one of the first or second antigen binding domains is a NKp46 antigen binding domain. Any suitable NKp46 antigen binding domain (as described herein and in the Figures, or a variant thereof) can be included in the subject 1+1 CLC format antibody.

In some embodiments, one of the first or second antigen binding domains is a MICA/B antigen binding domain. Any suitable MICA/B antigen binding domain (as described herein and in the Figures, or a variant thereof) can be included in the subject 1+1 CLC format antibody.

### 7. 2+1 CLC Format

One heterodimeric scaffold that finds particular use in the antibodies described herein is the “2+1 common light chain” (or (“2+1 CLC”) format. The 2+1 CLC format includes: a first monomer that includes a VH1-CH1-linker-VH1-CH1-hinge-CH2-CH3, wherein the first and second VH1 are each a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer that includes a “common light chain” V_{L}-C_{L}, wherein V_{L} is a common variable light domain and C_{L} is a constant light domain. The V_{L} pairs with each of the VH1s of the first monomer to form two first binding domains, each with a first antigen binding specificity; and the V_{L} pairs with the VH2 to form a second binding domain with a second antigen binding specificity. The linker of the first monomer can be any suitable linker, including, but not limited to, any one of the domain linkers described in FIG. 9 (see, e.g., SEQ ID NOs: 49-77). In some embodiments, the linker is EPKSCGKPGSGKPGS (SEQ ID NO: 6). In some embodiments, the 2+1 CLC format antibody is a trivalent antibody.

In some embodiments, the first and/or second Fc domains of the 2+1 CLC format are variant Fc domains. Generally, variant Fc domains can comprise one or more of: (a) one or more Fc ADCC variants (see, e.g., FIGS. 7 and 10), (b) one or more skew variants (see, e.g., FIGS. 4, 10, and 11), (c) one or more ablation variants (see, e.g., FIG. 6), (d) one or more FcRn variants (e.g., M428L, N434S, N434A, M428L/N434S, M428L/N434A, etc.), (e) a scFv linker (including those shown in FIG. 8; see, e.g., SEQ ID NOs: 23-48), and one or both of the heavy chains can comprise one or more pI variants (see, e.g., FIGS. 4, 5, and 10). The various types of variants can be used alone, or in combination with other variant types described herein.

In some embodiments, one or both of the Fc domains are configured such that one or both of the Fc domains have wildtype FcγR effector function. In some embodiments, the heterodimeric Fc backbone pairs have wildtype FcγR effector function, including, but not limited to, those pairs of Fc backbones shown in the Figures.

In some embodiments, one or both of the Fc domains include one or more amino acid substitutions such that one or both of the Fc domains have increased ADCC activity. In certain embodiments, the amino acid substitutions include one or more of: S239D, 1332E, or S239D/I332E, or any of the Fc ADCC variants shown in FIGS. 7 and 10. In some instances, the Fc backbone pair comprises an asymmetric, ADCC-enhanced heterodimeric Fc backbone pair (as described above). In other embodiments, the Fc backbone pair comprises a symmetric, ADCC-enhanced heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased ADCC activity, including, but not limited to, those pairs of Fc backbones shown in the Figures. In some embodiments, one or both of the Fc domains include one or more amino acid substitutions such that one or both of the Fc domains have increased ADCC activity, and optionally one or more of: (i) one or more skew variants, (ii) one or more ablation variants, (iii) one or more variants that improve serum half-life, (iv) one or more pI variants in the constant domain of the first and/or second monomer, or (v) any combination thereof.

In some embodiments, one or both of the Fc domains include heterodimerization skew variants (e.g., a set of amino acid substitutions, including, but not limited to, those shown in FIGS. 4, 10, and 11). Particularly useful heterodimerization skew variants include, but are not limited to: (i) S364K/E357Q: L368D/K370S, (ii) L368D/K370S: S364K, (iii) L368E/K370S: S364K, (iv) T411T/K360E/Q362E: D401K, (v) L368D/K370S: S364K/E357L, (vi) K370S: S364K/E357Q, (vii) T366S/L368A/Y407V: T366W, and (viii) T366S/L368A/Y407V/Y349C: T366W/S354C (according to EU numbering). In exemplary embodiments, one of the first or second variant Fc domains includes the heterodimerization skew variants L368D/K370S, and the other of the second or first variant Fc domain includes heterodimerization skew variants S364K/E357Q, wherein numbering is according to EU numbering. In some instances, the heterodimerization skew variants are in an asymmetric configuration, as described above. In other embodiments, the heterodimerization skew variants are in a symmetric configuration, as described above. In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, one or both of the Fc domains include heterodimerization skew variants, and optionally one or more of: (i) one or more Fc ADCC variants, (ii) one or more ablation variants, (iii) one or more variants that improve serum half-life, (iv) one or more pI variants in the constant domain of the first and/or second monomer, or (v) any combination thereof.

In some embodiments, one or both of the Fc domains include one or more amino acid substitutions such that one or both of the Fc domains have improved serum half-life. In certain embodiments, the amino acid substitutions include one or more of: M428L, N434S, N434A, M428L/N434S, M252Y/S254T/T256E, or M428L/N434A. In some instances, the Fc backbone pair comprises an asymmetric, Xtend heterodimeric Fc backbone pair (as described above). In other embodiments, the Fc backbone pair comprises a symmetric, Xtend heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased serum half-life, including, but not limited to, those pairs of Fc backbones shown in the Figures. In some embodiments, one or both of the Fc domains include one or more amino acid substitutions such that one or both of the Fc domains have improved serum half-life, and optionally one or more of: (i) one or more skew variants, (ii) one or more ablation variants, (iii) one or more Fc ADCC variants, (iv) one or more pI variants in the constant domain of the first and/or second monomer, or (v) any combination thereof.

In some embodiments, one or both of the Fc domains include one or more amino acid substitutions such that one or both of the Fc domains have an ablated FcγR function. Nonlimiting examples of amino acid substitutions (and combinations thereof) conferring an ablated FcγR function are shown in, for example, FIG. 6. In some instances, the Fc backbone pair comprises an asymmetric FcKO heterodimeric Fc backbone pair (as described above). In other embodiments, the Fc backbone pair comprises a symmetric, FcKO heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have ablated FcγR function, including, but not limited to, those pairs of Fc backbones shown in the Figures. In some embodiments, each of the first and second Fc domains include the ablation variants E233P/L234V/L235A/G236_/S267K, wherein numbering is according to EU numbering. In some embodiments, one or both of the Fc domains include one or more amino acid substitutions such that one or both of the Fc domains have an ablated FcγR function, and optionally one or more of: (i) one or more skew variants, (ii) one or more Fc ADCC variants, (iii) one or more variants that improve serum half-life, (iv) one or more pI variants in the constant domain of the first and/or second monomer, or (v) any combination thereof.

In some embodiments, the constant domain (CH1-hinge-CH2-CH3) of the first and/or second monomer includes pI variants (including, but not limited to, those shown in FIGS. 4, 5, and 10). In exemplary embodiments, the constant domain (CH1-hinge-CH2-CH3) of the first or second monomer includes pI variants N208D/Q295E/N384D/Q418E/N421D, wherein numbering is according to EU numbering. In some embodiments, the constant domain of the first and/or second monomer includes pI variants (including, but not limited to, those shown in FIGS. 4, 5, and 10), and the first and/or second Fc domain optionally contains one or more of: (i) one or more skew variants, (ii) one or more ablation variants, (iii) one or more variants that improve serum half-life, (iv) one or more Fc ADCC variants, or (v) any combination thereof.

In some embodiments, each of the first antigen binding domains or the second antigen binding domain is a NKp46 antigen binding domain. Any suitable NKp46 antigen binding domain (as described herein and in the Figures, or a variant thereof) can be included in the subject 2+1 CLC format antibody.

In some embodiments, each of the first antigen binding domains or the second antigen binding domain is a MICA/B antigen binding domain. Any suitable MICA/B antigen binding domain (as described herein and in the Figures, or a variant thereof) can be included in the subject 2+1 CLC format antibody.

### 8. mAb-Fv Format

One heterodimeric scaffold that finds particular use in the antibodies described herein is the “mAb-Fv” format. In this embodiment, the format relies on the use of a C-terminal attachment of an “extra” variable heavy domain to one monomer and the C-terminal attachment of an “extra” variable light domain to the other monomer, thus forming a third ABD (i.e., an “extra” Fv domain), wherein the Fab portions of the two monomers bind a MICA/B antigen and the “extra” scFv domain binds a NKp46. Alternatively, the Fab portions of the two monomers can bind a NKp46 antigen and the “extra” scFv domain can bind MICA/B.

In this embodiment, the first monomer comprises: a first heavy chain, comprising a first variable heavy domain and a first constant heavy domain comprising a first Fc domain, with a first variable light domain covalently attached to the C-terminus of the first Fc domain using a domain linker (VH1-CH1-hinge-CH2-CH3-[optional linker]-VL2). The second monomer comprises: a second variable heavy domain, a second constant heavy domain comprising a second Fc domain, and a third variable heavy domain covalently attached to the C-terminus of the second Fc domain using a domain linker (VH1-CH1-hinge-CH2-CH3-[optional linker]-VH2). This embodiment further utilizes a common light chain comprising a variable light domain and a constant light domain, which associates with the heavy chains to form two identical Fabs that include two identical Fvs. The two C-terminally attached variable domains (VL2 and VH2) make up the “extra” third Fv. As for many of the embodiments herein, these constructs can include one or more of: (i) Fc ADCC variants, (ii) pI variants, (iii) ablation variants, (iv) skew variants, (v) variants that improve serum half-life, as well as any combination thereof, as desired and described herein.

Any suitable NKp46 antigen binding domain (as described herein and in the Figures, or a variant thereof) can be included in the subject mAb-Fv format antibody.

Any suitable MICA/B antigen binding domain (as described herein and in the Figures, or a variant thereof) can be included in the subject mAb-Fv format antibody.

### 9. Dual scFv Format

The antibodies described herein also provide dual scFv formats as are known in the art. In this embodiment, the anti-NKp46×anti-MICA/B heterodimeric bispecific antibody is made up of two scFv-Fc monomers (both in either the V_{H}-scFv linker-V_{L}-[optional domain linker]-CH2-CH3 format, the V_{L}-scFv linker-V_{H}-[optional domain linker]-CH2-CH3 format, or with one monomer in one orientation and the other monomer in the other orientation).

In this case, all ABDs are in the scFv format. Any suitable NKp46 ABD and MICA/B ABD can be included in the subject bispecific antibodies in the dual scFv format, including any of the NKp46 ABDs and MICA/B ABDs described herein, and in the Figures, as well as variants thereof.

In some embodiments, the first and/or second Fc domains of the dual scFv format are variant Fc domains. Generally, variant Fc domains can comprise one or more of: (a) one or more Fc ADCC variants (see, e.g., FIGS. 7 and 10), (b) one or more skew variants (see, e.g., FIGS. 4, 10, and 11), (c) one or more ablation variants (see, e.g., FIG. 6), (d) one or more FcRn variants (e.g., M428L, N434S, N434A, M428L/N434S, M428L/N434A, etc.), (e) a scFv linker (including those shown in FIG. 8; see, e.g., SEQ ID NOs: 23-48), and one or both of the heavy chains can comprise one or more pI variants (see, e.g., FIGS. 4, 5, and 10). The various types of variants can be used alone, or in combination with other variant types described herein.

In some embodiments, one or both of the Fc domains are configured such that one or both of the Fc domains have wildtype FcγR effector function. In some embodiments, the heterodimeric Fc backbone pairs have wildtype FcγR effector function, including, but not limited to, those pairs of Fc backbones shown in the Figures.

In some embodiments, one or both of the Fc domains include one or more amino acid substitutions such that one or both of the Fc domains have increased ADCC activity. In certain embodiments, the amino acid substitutions include one or more of: S239D, 1332E, or S239D/I332E, or any of the Fc ADCC variants shown in FIGS. 7 and 10. In some instances, the Fc backbone pair comprises an asymmetric, ADCC-enhanced heterodimeric Fc backbone pair (as described above). In other embodiments, the Fc backbone pair comprises a symmetric, ADCC-enhanced heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased ADCC activity, including, but not limited to, those pairs of Fc backbones shown in the Figures. In some embodiments, one or both of the Fc domains include one or more amino acid substitutions such that one or both of the Fc domains have increased ADCC activity, and optionally one or more of: (i) one or more skew variants, (ii) one or more ablation variants, (iii) one or more variants that improve serum half-life, (iv) one or more pI variants in the constant domain of the first and/or second monomer, or (v) any combination thereof.

In some embodiments, one or both of the Fc domains include heterodimerization skew variants (e.g., a set of amino acid substitutions, including, but not limited to, those shown in FIGS. 4, 10, and 11). Particularly useful heterodimerization skew variants include, but are not limited to: (i) S364K/E357Q: L368D/K370S, (ii) L368D/K370S: S364K, (iii) L368E/K370S: S364K, (iv) T411T/K360E/Q362E: D401K, (v) L368D/K370S: S364K/E357L, (vi) K370S: S364K/E357Q, (vii) T366S/L368A/Y407V: T366W, and (viii) T366S/L368A/Y407V/Y349C: T366W/S354C (according to EU numbering). In exemplary embodiments, one of the first or second variant Fc domains includes the heterodimerization skew variants L368D/K370S, and the other of the second or first variant Fc domain includes heterodimerization skew variants S364K/E357Q, wherein numbering is according to EU numbering. In some instances, the heterodimerization skew variants are in an asymmetric configuration, as described above. In other embodiments, the heterodimerization skew variants are in a symmetric configuration, as described above. In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, one or both of the Fc domains include heterodimerization skew variants, and optionally one or more of: (i) one or more Fc ADCC variants, (ii) one or more ablation variants, (iii) one or more variants that improve serum half-life, (iv) one or more pI variants in the constant domain of the first and/or second monomer, or (v) any combination thereof.

In some embodiments, one or both of the Fc domains include one or more amino acid substitutions such that one or both of the Fc domains have improved serum half-life. In certain embodiments, the amino acid substitutions include one or more of: M428L, N434S, N434A, M428L/N434S, M252Y/S254T/T256E, or M428L/N434A. In some instances, the Fc backbone pair comprises an asymmetric, Xtend heterodimeric Fc backbone pair (as described above). In other embodiments, the Fc backbone pair comprises a symmetric, Xtend heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased serum half-life, including, but not limited to, those pairs of Fc backbones shown in the Figures. In some embodiments, one or both of the Fc domains include one or more amino acid substitutions such that one or both of the Fc domains have improved serum half-life, and optionally one or more of: (i) one or more skew variants, (ii) one or more ablation variants, (iii) one or more Fc ADCC variants, (iv) one or more pI variants in the constant domain of the first and/or second monomer, or (v) any combination thereof.

In some embodiments, one or both of the Fc domains include one or more amino acid substitutions such that one or both of the Fc domains have an ablated FcγR function. Nonlimiting examples of amino acid substitutions (and combinations thereof) conferring an ablated FcγR function are shown in, for example, FIG. 6. In some instances, the Fc backbone pair comprises an asymmetric FcKO heterodimeric Fc backbone pair (as described above). In other embodiments, the Fc backbone pair comprises a symmetric, FcKO heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have ablated FcγR function, including, but not limited to, those pairs of Fc backbones shown in the Figures. In some embodiments, each of the first and second Fc domains include the ablation variants E233P/L234V/L235A/G236_/S267K, wherein numbering is according to EU numbering. In some embodiments, one or both of the Fc domains include one or more amino acid substitutions such that one or both of the Fc domains have an ablated FcγR function, and optionally one or more of: (i) one or more skew variants, (ii) one or more Fc ADCC variants, (iii) one or more variants that improve serum half-life, (iv) one or more pI variants in the constant domain of the first and/or second monomer, or (v) any combination thereof.

In some embodiments, the constant domain (CH1-hinge-CH2-CH3) of the first and/or second monomer includes pI variants (including, but not limited to, those shown in FIGS. 4, 5, and 10). In exemplary embodiments, the constant domain (CH1-hinge-CH2-CH3) of the first or second monomer includes pI variants N208D/Q295E/N384D/Q418E/N421D, wherein numbering is according to EU numbering. In some embodiments, the constant domain of the first and/or second monomer includes pI variants (including, but not limited to, those shown in FIGS. 4, 5, and 10), and the first and/or second Fc domain optionally contains one or more of: (i) one or more skew variants, (ii) one or more ablation variants, (iii) one or more variants that improve serum half-life, (iv) one or more Fc ADCC variants, or (v) any combination thereof.

In some embodiments, the first scFv or the second scFv is the domain that binds to the NKp46 antigen. Any suitable NKp46 V_{H}/V_{L} pairs described herein (and in the Figures), or a variant thereof, can be included in the subject dual scFv format antibody.

In some embodiments, the first scFv or the second scFv is the domain that binds to the MICA/B antigen. Any suitable MICA/B V_{H}/V_{L} pairs described herein (and in the Figures), or a variant thereof, can be included in the subject dual scFv format antibody.

### 10. One-Armed scFv-mAb Format

One heterodimeric scaffold that finds particular use in the antibodies described herein is the “one-armed scFv-mAb” format. This format includes: 1) a first monomer that comprises an “empty” Fc domain; 2) a second monomer that includes a first variable heavy domain (V_{H}), a scFv domain (a second ABD), an Fc domain, where the scFv domain is attached to the N-terminus of the first variably heavy domain; and 3) a light chain that includes a first variable light domain and a constant light domain. The first variable heavy domain and the first variable light domain form a first antigen binding domain and the scFv is a second antigen binding domain. In this format, one of the first ABDs and the second ABDs binds a NKp46 antigen, and the other ABD binds a MICA/B antigen. As for many of the embodiments herein, these constructs can include one or more of: (i) Fc ADCC variants, (ii) pI variants, (iii) ablation variants, (iv) skew variants, (v) variants that improve serum half-life, as well as any combination thereof, as desired and described herein.

Any suitable NKp46 antigen binding domain (as described herein and in the Figures, or a variant thereof) can be included in the subject one-armed scFv-mAb format antibody.

Any suitable MICA/B antigen binding domain (as described herein and in the Figures, or a variant thereof) can be included in the subject one-armed scFv-mAb format antibody.

### 11. Central Fv Format

One heterodimeric scaffold that finds particular use in the antibodies described herein is the “central-Fv” format. In this embodiment, the format relies on the use of an inserted Fv domain (i.e., the central Fv domain) thus forming an “extra” third ABD, wherein the Fab portions of the two monomers bind a MICA/B antigen and the “extra” central Fv domain binds a NKp46 antigen. Alternatively, the Fab portions of the two monomers can bind a NKp46 antigen and the “extra” central Fv domain binds a MICA/B antigen. The Fv domain is inserted between the Fc domain and the CH1-Fv region of the monomers, thus providing a third ABD, wherein each monomer contains a component of the Fv (e.g., one monomer comprises a variable heavy domain and the other comprises a variable light domain of the “extra” central Fv domain).

In this embodiment, one monomer comprises a first heavy chain comprising a first variable heavy domain, a CH1 domain, and Fc domain, and an additional variable light domain. The additional variable light domain is covalently attached between the C-terminus of the CH1 domain of the heavy constant domain and the N-terminus of the first Fc domain using domain linkers (VH1-CH1-[optional linker]-VL2-hinge-CH2-CH3). The other monomer comprises a first heavy chain comprising a first variable heavy domain, a CH1 domain and Fc domain, and an additional variable heavy domain (VH1-CH1-[optional linker]-VH2-hinge-CH2-CH3). The additional variable heavy domain is covalently attached between the C-terminus of the CH1 domain of the heavy constant domain and the N-terminus of the first Fc domain using domain linkers. This embodiment further utilizes a common light chain comprising a variable light domain and a constant light domain, that associates with the heavy chains to form two identical Fabs that each bind a MICA/B antigen. The additional variable heavy domain and additional variable light domain form an “extra” central Fc that binds a NKp46 antigen. As for many of the embodiments herein, these constructs can include one or more of: (i) Fc ADCC variants, (ii) pI variants, (iii) ablation variants, (iv) skew variants, (v) variants that improve serum half-life, as well as any combination thereof, as desired and described herein.

Any suitable NKp46 antigen binding domain (as described herein and in the Figures, or a variant thereof) can be included in the subject central-Fv format antibody.

Any suitable MICA/B antigen binding domain (as described herein and in the Figures, or a variant thereof) can be included in the subject central-Fv format antibody.

### 12. scFv-mAb Format

One heterodimeric scaffold that finds particular use in the antibodies described herein is the “scFv-mAb” format. In this embodiment, the format relies on the use of a N-terminal attachment of a scFv to one of the monomers, thus forming a third ABD, wherein the Fab portions of the two monomers bind a MICA/B antigen and the “extra” scFv domain binds a NKp46 antigen. Alternatively, the Fab portions of the two monomers can bind a NKp46 antigen and the “extra” scFv domain can bind a MICA/B antigen.

In this embodiment, the first monomer comprises a first heavy chain (comprising a variable heavy domain and a constant domain), with a N-terminally covalently attached scFv comprising a scFv variable light domain, an scFv linker, and a scFv variable heavy domain in either orientation ((VH1-scFv linker-VL1-[optional domain linker]-VH2-CH1-hinge-CH2-CH3) or (with the scFv in the opposite orientation (VL1-scFv linker-VH1-[optional domain linker]-VH2-CH1-hinge-CH2-CH3))). This embodiment further utilizes a common light chain comprising a variable light domain and a constant light domain that associates with the heavy chains to form two identical Fabs that bind a MICA/B antigen. As for many of the embodiments herein, these constructs can include one or more of: (i) Fc ADCC variants, (ii) pI variants, (iii) ablation variants, (iv) skew variants, (v) variants that improve serum half-life, as well as any combination thereof, as desired and described herein.

Any suitable NKp46 antigen binding domain (as described herein and in the Figures, or a variant thereof) can be included in the subject scFv-mAb format antibody.

Any suitable MICA/B antigen binding domain (as described herein and in the Figures, or a variant thereof) can be included in the subject scFv-mAb format antibody.

### 13. Non-Heterodimeric Bispecific Antibodies

As will be appreciated by those in the art, the anti-NKp46×anti-MICA/B antibodies provided herein can also be included in non-heterodimeric bispecific formats. In this format, the anti-NKp46×anti-MICA/B antibody includes: (i) a first monomer comprising a VH1-CH1-hinge-CH2-CH3, (ii) a second monomer comprising a VH2-CH1-hinge-CH2-CH3, (iii) a first light chain comprising a VL1-C_{L}, and (iv) a second light chain comprising a VL2-C_{L}. In such embodiments, the VH1 and VL1 form a first ABD, and VH2 and VL2 form a second ABD. One of the first or second ABDS binds a MICA/B antigen, and the other ABD binds a NKp46 antigen.

Any suitable NKp46 antigen binding domain (as described herein and in the Figures, or a variant thereof) can be included in the subject non-heterodimeric bispecific format antibody.

Any suitable MICA/B antigen binding domain (as described herein and in the Figures, or a variant thereof) can be included in the subject non-heterodimeric bispecific format antibody.

### 14. Trident Format

In some embodiments, the bispecific antibodies described herein are in the “Trident” format as generally described in WO2015/184203, hereby expressly incorporated by reference in its entirety and in particular for the Figures, Legends, definitions, and sequences of “Heterodimer-Promoting Domains” or “HPDs,” including “K-coil” and “E-coil” sequences, and as are shown in FIG. 25M herein. Tridents rely on using two different HPDs that associate to form a heterodimeric structure as a component of the structure. In this embodiment, the Trident format includes a “traditional” heavy and light chain (e.g., VH1-CH1-hinge-CH2-CH3 and VL1-C_{L}), a third chain comprising a first “diabody-type binding domain” or “DART®,” VH2-(linker)-VL3-HPD1, and a fourth chain comprising a second DART®, VH3-(linker)-(linker)-VL2-HPD2. The VH1 and VL1 form a first ABD, the VH2 and VL2 form a second ABD, and the VH3 and VL3 form a third ABD. In some cases, the second and third ABDs bind the same antigen, in this instance generally a MICA/B antigen, e.g., bivalently, with the first ABD binding a NKp46 antigen monovalently. In other cases, the second and third ABDs bind the same antigen, in this instance generally a NKp46 antigen, e.g., bivalently, with the first ABD binding a MICA/B antigen monovalently.

Any suitable NKp46 antigen binding domain (as described herein and in the Figures, or a variant thereof) can be included in the subject Trident format antibody.

Any suitable MICA/B antigen binding domain (as described herein and in the Figures, or a variant thereof) can be included in the subject Trident format antibody.

### 15. Monospecific, Monoclonal Antibodies

As will be appreciated by those in the art, the novel MICA/B ABD sequences outlined herein can also be used in both monospecific antibodies (e.g., “traditional monoclonal antibodies”) or non-heterodimeric bispecific formats. Accordingly, in some embodiments, the antibodies described herein provide monoclonal (monospecific) antibodies comprising the 6 CDRs and/or the vh and vl sequences from the figures, generally with IgG1, IgG2, or IgG4 constant regions, with IgG1, IgG2 and IgG4 (including IgG4 constant regions comprising a S228P amino acid substitution) finding particular use in some embodiments. That is, any sequence herein with a “H_L” designation can be linked to the constant region of a human IgG1 antibody.

Any suitable MICA/B ABD can be included in the monospecific antibody, including any of the MICA/B ABDs described herein (see, e.g., FIGS. 21 and 22). In some embodiments, the monospecific antibody is a MICA/B monospecific antibody that has a V_{H}_V_{L} pair selected from the group including: (i) SEQ ID Nos: 239-246 for D94837_1E11_1 [MICA/B]_H0_D94837_E11_1 [MICA/B]_L0, (ii) SEQ ID Nos: 247-254 for D94837_1E11_1 [MICA/B]_H1_D94837_1E11_1 [MICA/B]_L1, (iii) SEQ ID Nos: 255-262 for D94837_1E11_1 [MICA/B]_H1_D94837_E11_1 [MICA/B]_L2, (iv) SEQ ID Nos: 263-270 for D94837_1E11_1 [MICA/B]_H2_D94837_E11_1 [MICA/B]_L1, (v) SEQ ID Nos: 271-278 for D94837_1E1_1 [MICA/B]_H2_D94837_E11_1 [MICA/B]_L2, (vi) SEQ ID Nos: 279-286 for 2E5 [MICA/B]_H0_2E5 [MICA/B]_L0, (vii) SEQ ID Nos: 287-294 for 2E5 [MICA/B]_H1_2E5 [MICA/B]_L1, (viii) SEQ ID Nos: 295-302 for 2E5 [MICA/B]_H1_2E5 [MICA/B]_L2, (ix) SEQ ID Nos: 303-310 for 2E5 [MICA/B]_H2_2E5 [MICA/B]_L1, (x) SEQ ID Nos: 311-318 for 2E5 [MICA/B]_H2_2E5 [MICA/B]_L2, (xi) SEQ ID Nos: 319-326 for D94852_2E12 [MICA/B]_H0_D94852_2E12 [MICA/B]_L0, (xii) SEQ ID Nos: 327-334 for D94852_2E12 [MICA/B]_H1_D94852_2E12 [MICA/B]_L1, (xiii) SEQ ID Nos: 335-342 for D94852_2E12 [MICA/B]_H1_D94852_2E12 [MICA/B]_L2, (xiv) SEQ ID Nos: 343-350 for D94852_2E12 [MICA/B]_H2_D94852_2E12 [MICA/B]_L1, (xv) SEQ ID Nos: 351-358 for D94852_2E12 [MICA/B]_H2_D94852_2E12 [MICA/B]_L2, (xvi) SEQ ID Nos: 359-366 for D99136_2F7 [MICA/B]_H0_D99136_2F7 [MICA/B]_L0, (xvii) SEQ ID Nos: 367-374 for D99136_2F7 [MICA/B]_H1_D99136_2F7 [MICA/B]_L1, (xviii) SEQ ID Nos: 375-382 for D99136_2F7 [MICA/B]_H1_D99136_2F7 [MICA/B]_L2, (xix) SEQ ID Nos: 383-390 for D99136_2F7 [MICA/B]_H2_D99136_2F7 [MICA/B]_L1, (xx) SEQ ID Nos: 391-398 for D99136_2F7 [MICA/B]_H2_D99136_2F7 [MICA/B]_L2, (xxi) SEQ ID Nos: 399-406 for D103388_1C7 [MICA/B]_H0_D103388_1C7 [MICA/B]_L0, (xxii) SEQ ID Nos: 407-414 for D103388_1C7 [MICA/B]_H1_D103388_1C7 [MICA/B]_L1, (xxiii) SEQ ID Nos: 415-422 for D103388_1C7 [MICA/B]_H1_D103388_1C7 [MICA/B]_L2, (xxiv) SEQ ID Nos: 423-430 for D103388_1C7 [MICA/B]_H2_D103388_1C7 [MICA/B]_L1, (xxv) SEQ ID Nos: 431-438 for D103388_1C7 [MICA/B]_H2_D103388_1C7 [MICA/B]_L2, (xxvi) SEQ ID Nos: 439-446 for D103388_1D7 [MICA/B]_H0_D103388_1D7 [MICA/B]_L0, (xxvii) SEQ ID Nos: 447-454 for D103388_1D7 [MICA/B]_H1_D103388_1D7 [MICA/B]_L1, (xxviii) SEQ ID Nos: 455-462 for D103388_1D7 [MICA/B]_H1_D103388_1D7 [MICA/B]_L2, (xxix) SEQ ID Nos: 463-470 for D103388_1D7 [MICA/B]_H2_D103388_1D7 [MICA/B]_L1, (xxx) SEQ ID Nos: 471-478 for D103388_1D7 [MICA/B]_H2_D103388_1D7 [MICA/B]_L2, (xxxi) SEQ ID Nos: 479-486 for D105317_1A2 [MICA/B]_H0_D105317_1A2 [MICA/B]_L0, (xxxii) SEQ ID Nos: 487-494 for D105317_1A2 [MICA/B]_H1_D105317_1A2 [MICA/B]_L1, (xxxiii) SEQ ID Nos: 495-502 for D105317_1A2 [MICA/B]_H1_D105317_1A2 [MICA/B]_L2, (xxxiv) SEQ ID Nos: 503-510 for D105317_1A2 [MICA/B]_H2_D105317_1A2 [MICA/B]_L1, (xxxv) SEQ ID Nos: 511-518 for D105317_1A2 [MICA/B]_H2_D105317_1A2 [MICA/B]_L2, (xxxvi) SEQ ID Nos: 519-526 for D99136_2C11 [MICA/B]_H0_D99136_2C11 [MICA/B]_L0, (xxxvii) SEQ ID Nos: 527-534 for D99136_2C11 [MICA/B]_H1_D99136_2C11 [MICA/B]_L1, (xxxviii) SEQ ID Nos: 535-542 for D99136_2C11 [MICA/B]_H1_D99136_2C11 [MICA/B]_L2, (xxxix) SEQ ID Nos: 543-550 for D99136_2C11 [MICA/B]_H2_D99136_2C11 [MICA/B]_L1, (xl) SEQ ID Nos: 551-558 for D99136_2C11 [MICA/B]_H2_D99136_2C11 [MICA/B]_L2, (xli) SEQ ID Nos: 559-566 for D103388_1B11-2 [MICA/B]_H0_D103388_1B11-2 [MICA/B]_L0, (xlii) SEQ ID Nos: 567-574 for D105317_1B6 [MICA/B]_H0_D105317_1B6 [MICA/B]_L0, (xliii) SEQ ID Nos: 575-582 for D105317_1C8 [MICA/B]_H0_D105317_1C8 [MICA/B]_L0, (xliv) SEQ ID Nos: 583-590 for D105317_1F6 [MICA/B]_H0_D105317_1F6 [MICA/B]_L0, (xlv) SEQ ID Nos: 591-598 for D105317_1F7 [MICA/B]_H0_D105317_1F7 [MICA/B]_L0, (xlvi) SEQ ID Nos: 599-606 for D94837_1D3 [MICA/B]_H0_D94837_1D3 [MICA/B]_L0, (xlvii) SEQ ID Nos: 607-614 for D94837_1D8 [MICA/B]_H0_D94837_1D8 [MICA/B]_L0, (xlviii) SEQ ID Nos: 615-622 for D94837_1D9 [MICA/B]_H0_D94837_1D9 [MICA/B]_L0, (xlix) SEQ ID Nos: 623-630 for D94837_1E1 [MICA/B]_H0_D94837_1E1 [MICA/B]_L0, (1) SEQ ID Nos: 631-638 for D94852_2D4 [MICA/B]_H0_D94852_2D4 [MICA/B]_L0, (li) SEQ ID Nos: 639-646 for D94852_2G8 [MICA/B]_H0_D94852_2G8 [MICA/B]_L0, (lii) SEQ ID Nos: 647-654 for D88487_2A8 [MICA/B]_H0_D88487_2A8 [MICA/B]_L0, (liii) SEQ ID Nos: 655-662 for D94837_3B12 [MICA/B]_H0_D94837_3B12 [MICA/B]_L0, (liv) SEQ ID Nos: 663-670 for D99122_1H7 [MICA/B]_H0_D99122_1H7 [MICA/B]_L0, (lv) SEQ ID Nos: 671-678 for D99136_2E8 [MICA/B]_H0_D99136_2E8 [MICA/B]_L0, (lvi) SEQ ID Nos: 647-654 for D88487_2A8 [MICA/B]_H0_D88487_2A8 [MICA/B]_L0, (lvii) SEQ ID Nos: 679-686 for 3F9[MICA/B]_H0_3F9[MICA/B]_L0, (lviii) SEQ ID Nos: 687-694 for 6E1[MICA/B]_H0_6E1[MICA/B]_L0, (lix) SEQ ID Nos: 695-702 for 7C6[MICA/B]_H0_7C6[MICA/B]_L0, (lx) SEQ ID Nos: 703-710 for 13A9 [MICA/B]_H0_13A9 [MICA/B]_L0, and (lxi) SEQ ID Nos: 711-718 for 1D5 [MICA/B]_H0_1D5 [MICA/B]_L0, or variants thereof (see, e.g., FIGS. 21 and 22).

In particular monoclonal embodiments, the V_{H}_V_{L} pair is selected from the group including: (i) D94837_1E11_1 [MICA/B]_H0_D94837_1E11_1 [MICA/B]_L0 (SEQ ID NOs: 239-246), (ii) D94837_1E11_1 [MICA/B]_H1_D94837_1E11_1 [MICA/B]_L1 (SEQ ID NOs: 247-254), (iii) D94837_1E11_1 [MICA/B]_H1_D94837_1E11_1 [MICA/B]_L2 (SEQ ID NOs: 255-262), (iv) D94837_1E11_1 [MICA/B]_H2_D94837_1E11_1 [MICA/B]_L1 (SEQ ID NOs: 263-270), (v) D94837_1E11_1 [MICA/B]_H2_D94837_1E11_1 [MICA/B]_L2 (SEQ ID NOs: 271-278), (vi) 2E5 [MICA/B]_H0_2E5 [MICA/B]_L0 (SEQ ID NOs: 279-286), (vii) 2E5 [MICA/B]_H1_2E5 [MICA/B]_L1 (SEQ ID NOs: 287-294), (viii) 2E5 [MICA/B]_H1_2E5 [MICA/B]_L2 (SEQ ID NOs: 295-302), (ix) 2E5 [MICA/B]_H2_2E5 [MICA/B]_L1 (SEQ ID NOs: 303-310), (x) 2E5 [MICA/B]_H2_2E5 [MICA/B]_L2 (SEQ ID NOs: 311-318), (xi) D94852_2E12 [MICA/B]_H0_D94852_2E12 [MICA/B]_L0 (SEQ ID NOs: 319-326), (xii) D94852_2E12 [MICA/B]_H1_D94852_2E12 [MICA/B]_L1 (SEQ ID NOs: 327-334), (xiii) D94852_2E12 [MICA/B]_H1_D94852_2E12 [MICA/B]_L2 (SEQ ID NOs: 335-342), (xiv) D94852_2E12 [MICA/B]_H2_D94852_2E12 [MICA/B]_L1 (SEQ ID NOs: 343-350), (xv) D94852_2E12 [MICA/B]_H2_D94852_2E12 [MICA/B]_L2 (SEQ ID NOs: 351-358), (xvi) D99136_2F7 [MICA/B]_H0_D99136_2F7 [MICA/B]_L0 (SEQ ID NOs: 359-366), (xvii) D99136_2F7 [MICA/B]_H1_D99136_2F7 [MICA/B]_L1 (SEQ ID NOs: 367-374), (xviii) D99136_2F7 [MICA/B]_H1_D99136_2F7 [MICA/B]_L2 (SEQ ID NOs: 375-382), (xix) D991362F7 [MICA/B]_H2_D99136_2F7 [MICA/B]_L1 (SEQ ID NOs: 383-390), (xx) D99136_2F7 [MICA/B]_H2_D99136_2F7 [MICA/B]_L2 (SEQ ID NOs: 391-398), (xxi) D103388_1C7 [MICA/B]_H0_D103388_1C7 [MICA/B]_L0 (SEQ ID NOs: 399-406), (xxii) D103388_1C7 [MICA/B]_H1_D103388_1C7 [MICA/B]_L1 (SEQ ID NOs: 407-414), (xxiii) D103388_1C7 [MICA/B]_H1_D103388_1C7 [MICA/B]_L2 (SEQ ID NOs: 415-422), (xxiv) D103388_1C7 [MICA/B]_H2_D103388_1C7 [MICA/B]_L1 (SEQ ID NOs: 423-430), (xxv) D103388_1C7 [MICA/B]_H2_D103388_1C7 [MICA/B]_L2 (SEQ ID NOs: 431-438), (xxvi) D103388_1D7 [MICA/B]_H0_D103388_1D7 [MICA/B]_L0 (SEQ ID NOs: 439-446), (xxvii) D103388_1D7 [MICA/B]_H1_D103388_1D7 [MICA/B]_L1 (SEQ ID NOs: 447-454), (xxviii) D103388_1D7 [MICA/B]_H1_D103388_1D7 [MICA/B]_L2 (SEQ ID NOs: 455-462), (xxix) D103388_1D7 [MICA/B]_H2_D103388_1D7 [MICA/B]_L1 (SEQ ID NOs: 463-470), (xxx) D103388_1D7 [MICA/B]_H2_D103388_1D7 [MICA/B]_L2 (SEQ ID NOs: 471-478), (xxxi) D105317_1A2 [MICA/B]_H0_D105317_1A2 [MICA/B]_L0 (SEQ ID NOs: 479-486), (xxxii) D105317_1A2 [MICA/B]_H1_D105317_1A2 [MICA/B]_L1 (SEQ ID NOs: 487-494), (xxxiii) D105317_1A2 [MICA/B]_H1_D105317_1A2 [MICA/B]_L2 (SEQ ID NOs: 495-502), (xxxiv) D105317_1A2 [MICA/B]_H2_D105317_1A2 [MICA/B]_L1 (SEQ ID NOs: 503-510), (xxxv) D105317_1A2 [MICA/B]_H2_D105317_1A2 [MICA/B]_L2 (SEQ ID NOs: 511-518), (xxxvi) D99136_2C11 [MICA/B]_H0_D99136_2C11 [MICA/B]_L0 (SEQ ID NOs: 519-526), (xxxvii) D99136_2C11 [MICA/B]_H1_D99136_2C11 [MICA/B]_L1 (SEQ ID NOs: 527-534), (xxxviii) D99136_2C11 [MICA/B]_H1_D99136_2C11 [MICA/B]_L2 (SEQ ID NOs: 535-542), (xxxix) D99136_2C11 [MICA/B]_H2_D99136_2C11 [MICA/B]_L1 (SEQ ID NOs: 543-550), (xl) D99136_2C11 [MICA/B]_H2_D99136_2C11 [MICA/B]_L2 (SEQ ID NOs: 551-558), (xli) D103388_1B11-2 [MICA/B]_H0_D103388_1B11-2 [MICA/B]_L0 (SEQ ID NOs: 559-566), (xlii) D105317_1B6 [MICA/B]_H0_D105317_1B6 [MICA/B]_L0 (SEQ ID NOs: 567-574), (xliii) D105317_1C8 [MICA/B]_H0_D105317_1C8 [MICA/B]_L0 (SEQ ID NOs: 575-582), (xliv) D105317_1F6 [MICA/B]_H0_D105317_1F6 [MICA/B]_L0 (SEQ ID NOs: 583-590), (xlv) D105317_1F7 [MICA/B]_H0_D105317_1F7 [MICA/B]_L0 (SEQ ID NOs: 591-598), (xlvi) D94837_1D3 [MICA/B]_H0_D94837_1D3 [MICA/B]_L0 (SEQ ID NOs: 599-606), (xlvii) D94837_1D8 [MICA/B]_H0_D94837_1D8 [MICA/B]_L0 (SEQ ID NOs: 607-614), (xlviii) D94837_1D9 [MICA/B]_H0_D94837_1D9 [MICA/B]_L0 (SEQ ID NOs: 615-622), (xlix) D94837_1E1 [MICA/B]_H0_D94837_1E1 [MICA/B]_L0 (SEQ ID NOs: 623-630), (1) D94852_2D4 [MICA/B]_H0_D94852_2D4 [MICA/B]_L0 (SEQ ID NOs: 631-638), (li) D94852_2G8 [MICA/B]_H0_D94852_2G8 [MICA/B]_L0 (SEQ ID NOs: 639-646), (lii) D88487_2A8 [MICA/B]_H0_D88487_2A8 [MICA/B]_L0 (SEQ ID NOs: 647-654), (liii) D94837_3B12 [MICA/B]_H0_D94837_3B12 [MICA/B]_L0 (SEQ ID NOs: 655-662), (liv) D99122_1H7 [MICA/B]_H0_D99122_1H7 [MICA/B]_L0 (SEQ ID NOs: 663-670), (lv) D99136_2E8 [MICA/B]_H0_D99136_2E8 [MICA/B]_L0 (SEQ ID NOs: 671-678), (lvi) D88487_2A8 [MICA/B]_H0_D88487_2A8 [MICA/B]_L0 (SEQ ID NOs: 647-654), (lvii) 3F9[MICA/B]_H0_3F9[MICA/B]_L0 (SEQ ID NOs: 679-686), (lviii) 6E1[MICA/B]_H0_6E1[MICA/B]_L0 (SEQ ID NOs: 687-694), (lix) 7C6[MICA/B]_H0_7C6[MICA/B]_L0 (SEQ ID NOs: 695-702), (lx) 13A9 [MICA/B]_H0_13A9 [MICA/B]_L0 (SEQ ID NOs: 703-710), and (lxi) 1D5 [MICA/B]_H0_1D5 [MICA/B]_L0 (SEQ ID NOs: 711-718).

Similarly, the novel NKp46 ABD sequences outlined herein can also be used in both monospecific antibodies (e.g., “traditional monoclonal antibodies”) or non-heterodimeric bispecific formats. Accordingly, in some embodiments, the antibodies described herein provide monoclonal (monospecific) antibodies comprising the 6 CDRs and/or the vh and vl sequences from the figures, generally with IgG1, IgG2, or IgG4 constant regions, with IgG1, IgG2 and IgG4 (including IgG4 constant regions comprising a S228P amino acid substitution) finding particular use in some embodiments. That is, any sequence herein with a “H_L” designation can be linked to the constant region of a human IgG1 antibody.

Any suitable NKp46 ABD can be included in the monospecific antibody, including any of the NKp46 ABDs described herein (see, e.g., FIGS. 23 and 24). In some embodiments, the monospecific antibody is a NKp46 monospecific antibody that has a V_{H}_V_{L} pair selected from the group including: (i) SEQ ID NOs: 719 and 723 for 2C10A3.372[NKp46]_H1_2C10A3.372[NKp46]_L1, and (ii) SEQ ID NOs: 727 and 731 for NKp46-A[NKp46]_H_NKp46-A[NKp46]_L, or variants thereof (see, e.g., FIGS. 23 and 24).

In particular monoclonal embodiments, the V_{H}_V_{L} pair is selected from the group including: 2C10A3.372[NKp46]_H1_2C10A3.372[NKp46]_L1 (SEQ ID NOs: 719 and 723), and NKp46-A[NKp46]_H_NKp46-A[NKp46]_L (SEQ ID NOs: 727 and 731).

### I. Particular Embodiments of the Invention with Increased Binding to FcγRIIIa

In particular 1+1 format embodiments, the αNKp46 ABD is the Fab and has the V_{H}_V_{L} pair selected from the group including: (i) 2C10A3.372[NKp46]_H1_2C10A3.372[NKp46]_L1 (SEQ ID NOs: 719-726), and (ii) NKp46-A[NKp46]_H_NKp46-A[NKp46]_L (SEQ ID NOs: 727-734); and the αMICA/B is a scFv selected from the group including: (i) D94837_1E11_1 [MICA/B]_H0_D94837_1E11_1 [MICA/B]_L0 (SEQ ID NOs: 239-246), (ii) D94837_1E11_1 [MICA/B]_H1_D94837_1E11_1 [MICA/B]_L1 (SEQ ID NOs: 247-254), (iii) D94837_1E11_1 [MICA/B]_H1_D94837_1E11_1 [MICA/B]_L2 (SEQ ID NOs: 255-262), (iv) D94837_1E11_1 [MICA/B]_H2_D94837_1E11_1 [MICA/B]_L1 (SEQ ID NOs: 263-270), (v) D94837_1E11_1 [MICA/B]_H2_D94837_1E11_1 [MICA/B]_L2 (SEQ ID NOs: 271-278), (vi) 2E5 [MICA/B]_H0_2E5 [MICA/B]_L0 (SEQ ID NOs: 279-286), (vii) 2E5 [MICA/B]_H1_2E5 [MICA/B]_L1 (SEQ ID NOs: 287-294), (viii) 2E5 [MICA/B]H1 2E5 [MICA/B]L2 (SEQ ID NOs: 295-302), (ix) 2E5 [MICA/B]_H2_2E5 [MICA/B]_L1 (SEQ ID NOs: 303-310), (x) 2E5 [MICA/B]_H2_2E5 [MICA/B]_L2 (SEQ ID NOs: 311-318), (xi) D94852_2E12 [MICA/B]_H0_D94852_2E12 [MICA/B]_L0 (SEQ ID NOs: 319-326), (xii) D94852_2E12 [MICA/B]_H1_D94852_2E12 [MICA/B]_L1 (SEQ ID NOs: 327-334), (xiii) D94852_2E12 [MICA/B]_H1_D94852_2E12 [MICA/B]_L2 (SEQ ID NOs: 335-342), (xiv) D94852_2E12 [MICA/B]_H2_D94852_2E12 [MICA/B]_L1 (SEQ ID NOs: 343-350), (xv) D94852_2E12 [MICA/B]_H2_D94852_2E12 [MICA/B]_L2 (SEQ ID NOs: 351-358), (xvi) D99136_2F7 [MICA/B]_H0_D99136_2F7 [MICA/B]_L0 (SEQ ID NOs: 359-366), (xvii) D99136_2F7 [MICA/B]_H1_D99136_2F7 [MICA/B]_L1 (SEQ ID NOs: 367-374), (xviii) D99136_2F7 [MICA/B]_H1_D99136_2F7 [MICA/B]_L2 (SEQ ID NOs: 375-382), (xix) D99136_2F7 [MICA/B]_H2_D99136_2F7 [MICA/B]_L1 (SEQ ID NOs: 383-390), (xx) D99136_2F7 [MICA/B]_H2_D99136_2F7 [MICA/B]_L2 (SEQ ID NOs: 391-398), (xxi) D103388_1C7 [MICA/B]_H0_D103388_1C7 [MICA/B]_L0 (SEQ ID NOs: 399-406), (xxii) D103388_1C7 [MICA/B]_H1_D103388_1C7 [MICA/B]_L1 (SEQ ID NOs: 407-414), (xxiii) D103388_1C7 [MICA/B]_H1_D103388_1C7 [MICA/B]_L2 (SEQ ID NOs: 415-422), (xxiv) D103388_1C7 [MICA/B]_H2_D103388_1C7 [MICA/B]_L1 (SEQ ID NOs: 423-430), (xxv) D103388_1C7 [MICA/B]_H2_D103388_1C7 [MICA/B]_L2 (SEQ ID NOs: 431-438), (xxvi) D103388_1D7 [MICA/B]_H0_D103388_1D7 [MICA/B]_L0 (SEQ ID NOs: 439-446), (xxvii) D103388_1D7 [MICA/B]_H1_D103388_1D7 [MICA/B]_L1 (SEQ ID NOs: 447-454), (xxviii) D103388_1D7 [MICA/B]_H1_D103388_1D7 [MICA/B]_L2 (SEQ ID NOs: 455-462), (xxix) D103388_1D7 [MICA/B]_H2_D103388_1D7 [MICA/B]_L1 (SEQ ID NOs: 463-470), (xxx) D103388_1D7 [MICA/B]_H2_D103388_1D7 [MICA/B]_L2 (SEQ ID NOs: 471-478), (xxxi) D105317_1A2 [MICA/B]_H0_D105317_1A2 [MICA/B]_L0 (SEQ ID NOs: 479-486), (xxxii) D105317_1A2 [MICA/B]_H1_D105317_1A2 [MICA/B]_L1 (SEQ ID NOs: 487-494), (xxxiii) D105317_1A2 [MICA/B]_H1_D105317_1A2 [MICA/B]_L2 (SEQ ID NOs: 495-502), (xxxiv) D105317_1A2 [MICA/B]_H2_D105317_1A2 [MICA/B]_L1 (SEQ ID NOs: 503-510), (xxxv) D105317_1A2 [MICA/B]_H2_D105317_1A2 [MICA/B]_L2 (SEQ ID NOs: 511-518), (xxxvi) D99136_2C11 [MICA/B]_H0_D99136_2C11 [MICA/B]_L0 (SEQ ID NOs: 519-526), (xxxvii) D99136_2C11 [MICA/B]_H1_D99136_2C11 [MICA/B]_L1 (SEQ ID NOs: 527-534), (xxxviii) D99136_2C11 [MICA/B]_H1_D99136_2C11 [MICA/B]_L2 (SEQ ID NOs: 535-542), (xxxix) D99136_2C11 [MICA/B]_H2_D99136_2C11 [MICA/B]_L1 (SEQ ID NOs: 543-550), (xl) D99136_2C11 [MICA/B]_H2_D99136_2C11 [MICA/B]_L2 (SEQ ID NOs: 551-558), (xli) D103388_1B11-2 [MICA/B]_H0_D103388_1B11-2 [MICA/B]_L0 (SEQ ID NOs: 559-566), (xlii) D105317_1B6 [MICA/B]_H0_D105317_1B6 [MICA/B]_L0 (SEQ ID NOs: 567-574), (xliii) D105317_1C8 [MICA/B]_H0_D105317_1C8 [MICA/B]_L0 (SEQ ID NOs: 575-582), (xliv) D105317_1F6 [MICA/B]_H0_D105317_1F6 [MICA/B]_L0 (SEQ ID NOs: 583-590), (xlv) D105317_1F7 [MICA/B]_H0_D105317_1F7 [MICA/B]_L0 (SEQ ID NOs: 591-598), (xlvi) D94837_1D3 [MICA/B]_H0_D94837_1D3 [MICA/B]_L0 (SEQ ID NOs: 599-606), (xlvii) D94837_1D8 [MICA/B]_H0_D94837_1D8 [MICA/B]_L0 (SEQ ID NOs: 607-614), (xlviii) D94837_1D9 [MICA/B]_H0_D94837_1D9 [MICA/B]_L0 (SEQ ID NOs: 615-622), (xlix) D94837_1E1 [MICA/B]_H0_D94837_1E1 [MICA/B]_L0 (SEQ ID NOs: 623-630), (1) D94852_2D4 [MICA/B]_H0_D94852_2D4 [MICA/B]_L0 (SEQ ID NOs: 631-638), (li) D94852_2G8 [MICA/B]_H0_D94852_2G8 [MICA/B]_L0 (SEQ ID NOs: 639-646), (lii) D88487_2A8 [MICA/B]_H0_D88487_2A8 [MICA/B]_L0 (SEQ ID NOs: 647-654), (liii) D94837_3B12 [MICA/B]_H0_D94837_3B12 [MICA/B]_L0 (SEQ ID NOs: 655-662), (liv) D99122_1H7 [MICA/B]_H0_D99122_1H7 [MICA/B]_L0 (SEQ ID NOs: 663-670), (lv) D99136_2E8 [MICA/B]_H0_D99136_2E8 [MICA/B]_L0 (SEQ ID NOs: 671-678), (lvi) D88487_2A8 [MICA/B]_H0_D88487_2A8 [MICA/B]_L0 (SEQ ID NOs: 647-654), (lvii) 3F9[MICA/B]_H0_3F9[MICA/B]_L0 (SEQ ID NOs: 679-686), (lviii) 6E1[MICA/B]_H0_6E1[MICA/B]_L0 (SEQ ID NOs: 687-694), (lix) 7C6[MICA/B]_H0_7C6[MICA/B]_L0 (SEQ ID NOs: 695-702), (lx) 13A9 [MICA/B]_H0_13A9 [MICA/B]_L0 (SEQ ID NOs: 703-710), and (lxi) 1D5 [MICA/B]_H0_1D5 [MICA/B]_L0 (SEQ ID NOs: 711-718).

In particular 1+1 format embodiments, the αMICA/B ABD is the Fab and has the V_{H}_V_{L} pair selected from the group including: (i) D94837_1E11_1 [MICA/B]_H0_D94837_1E11_1 [MICA/B]_L0 (SEQ ID NOs: 239-246), (ii) D94837_1E11_1 [MICA/B]_H1_D94837_1E11_1 [MICA/B]_L1 (SEQ ID NOs: 247-254), (iii) D94837_1E11_1 [MICA/B]_H1_D94837_1E11_1 [MICA/B]_L2 (SEQ ID NOs: 255-262), (iv) D94837_1E11_1 [MICA/B]_H2_D94837_1E11_1 [MICA/B]_L1 (SEQ ID NOs: 263-270), (v) D94837_1E11_1 [MICA/B]_H2_D94837_1E11_1 [MICA/B]_L2 (SEQ ID NOs: 271-278), (vi) 2E5 [MICA/B]_H0_2E5 [MICA/B]_L0 (SEQ ID NOs: 279-286), (vii) 2E5 [MICA/B]_H1_2E5 [MICA/B]_L1 (SEQ ID NOs: 287-294), (viii) 2E5 [MICA/B]_H1_2E5 [MICA/B]_L2 (SEQ ID NOs: 295-302), (ix) 2E5 [MICA/B]_H2_2E5 [MICA/B]_L1 (SEQ ID NOs: 303-310), (x) 2E5 [MICA/B]_H2_2E5 [MICA/B]_L2 (SEQ ID NOs: 311-318), (xi) D94852_2E12 [MICA/B]_H0_D94852_2E12 [MICA/B]_L0 (SEQ ID NOs: 319-326), (xii) D94852_2E12 [MICA/B]_H1_D94852_2E12 [MICA/B]_L1 (SEQ ID NOs: 327-334), (xiii) D94852_2E12 [MICA/B]_H1_D94852_2E12 [MICA/B]_L2 (SEQ ID NOs: 335-342), (xiv) D94852_2E12 [MICA/B]_H2_D94852_2E12 [MICA/B]_L1 (SEQ ID NOs: 343-350), (xv) D94852_2E12 [MICA/B]_H2_D94852_2E12 [MICA/B]_L2 (SEQ ID NOs: 351-358), (xvi) D99136_2F7 [MICA/B]_H0_D99136_2F7 [MICA/B]_L0 (SEQ ID NOs: 359-366), (xvii) D99136_2F7 [MICA/B]_H1_D99136_2F7 [MICA/B]_L1 (SEQ ID NOs: 367-374), (xviii) D99136_2F7 [MICA/B]_H1_D99136_2F7 [MICA/B]_L2 (SEQ ID NOs: 375-382), (xix) D99136_2F7 [MICA/B]_H2_D99136_2F7 [MICA/B]_L1 (SEQ ID NOs: 383-390), (xx) D99136_2F7 [MICA/B]_H2_D99136_2F7 [MICA/B]_L2 (SEQ ID NOs: 391-398), (xxi) D103388_1C7 [MICA/B]_H0_D103388_1C7 [MICA/B]_L0 (SEQ ID NOs: 399-406), (xxii) D103388_1C7 [MICA/B]_H1_D103388_1C7 [MICA/B]_L1 (SEQ ID NOs: 407-414), (xxiii) D103388_1C7 [MICA/B]_H1_D103388_1C7 [MICA/B]_L2 (SEQ ID NOs: 415-422), (xxiv) D103388_1C7 [MICA/B]_H2_D103388_1C7 [MICA/B]_L1 (SEQ ID NOs: 423-430), (xxv) D103388_1C7 [MICA/B]_H2_D103388_1C7 [MICA/B]_L2 (SEQ ID NOs: 431-438), (xxvi) D103388_1D7 [MICA/B]_H0_D103388_1D7 [MICA/B]_L0 (SEQ ID NOs: 439-446), (xxvii) D103388_1D7 [MICA/B]_H1_D103388_1D7 [MICA/B]_L1 (SEQ ID NOs: 447-454), (xxviii) D103388_1D7 [MICA/B]_H1_D103388_1D7 [MICA/B]_L2 (SEQ ID NOs: 455-462), (xxix) D103388_1D7 [MICA/B]_H2_D103388_1D7 [MICA/B]_L1 (SEQ ID NOs: 463-470), (xxx) D103388_1D7 [MICA/B]_H2_D103388_1D7 [MICA/B]_L2 (SEQ ID NOs: 471-478), (xxxi) D105317_1A2 [MICA/B]_H0_D105317_1A2 [MICA/B]_L0 (SEQ ID NOs: 479-486), (xxxii) D105317_1A2 [MICA/B]_H1_D105317_1A2 [MICA/B]_L1 (SEQ ID NOs: 487-494), (xxxiii) D105317_1A2 [MICA/B]_H1_D105317_1A2 [MICA/B]_L2 (SEQ ID NOs: 495-502), (xxxiv) D105317_1A2 [MICA/B]_H2_D105317_1A2 [MICA/B]_L1 (SEQ ID NOs: 503-510), (xxxv) D105317_1A2 [MICA/B]_H2_D105317_1A2 [MICA/B]_L2 (SEQ ID NOs: 511-518), (xxxvi) D99136_2C11 [MICA/B]_H0_D99136_2C11 [MICA/B]_L0 (SEQ ID NOs: 519-526), (xxxvii) D99136_2C11 [MICA/B]_H1_D99136_2C11 [MICA/B]_L1 (SEQ ID NOs: 527-534), (xxxviii) D99136_2C11 [MICA/B]_H1_D99136_2C11 [MICA/B]_L2 (SEQ ID NOs: 535-542), (xxxix) D99136_2C11 [MICA/B]_H2_D99136_2C11 [MICA/B]_L1 (SEQ ID NOs: 543-550), (xl) D99136_2C11 [MICA/B]_H2_D99136_2C11 [MICA/B]_L2 (SEQ ID NOs: 551-558), (xli) D103388_1B11-2 [MICA/B]_H0_D103388_1B11-2 [MICA/B]_L0 (SEQ ID NOs: 559-566), (xlii) D105317_1B6 [MICA/B]_H0_D105317_1B6 [MICA/B]_L0 (SEQ ID NOs: 567-574), (xliii) D105317_1C8 [MICA/B]_H0_D105317_1C8 [MICA/B]_L0 (SEQ ID NOs: 575-582), (xliv) D105317_1F6 [MICA/B]_H0_D105317_1F6 [MICA/B]_L0 (SEQ ID NOs: 583-590), (xlv) D105317_1F7 [MICA/B]_H0_D105317_1F7 [MICA/B]_L0 (SEQ ID NOs: 591-598), (xlvi) D94837_1D3 [MICA/B]_H0_D94837_1D3 [MICA/B]_L0 (SEQ ID NOs: 599-606), (xlvii) D94837_1D8 [MICA/B]_H0_D94837_1D8 [MICA/B]_L0 (SEQ ID NOs: 607-614), (xlviii) D94837_1D9 [MICA/B]_H0_D94837_1D9 [MICA/B]_L0 (SEQ ID NOs: 615-622), (xlix) D94837_1E1 [MICA/B]_H0_D94837_1E1 [MICA/B]_L0 (SEQ ID NOs: 623-630), (1) D94852_2D4 [MICA/B]_H0_D94852_2D4 [MICA/B]_L0 (SEQ ID NOs: 631-638), (li) D94852_2G8 [MICA/B]_H0_D94852_2G8 [MICA/B]_L0 (SEQ ID NOs: 639-646), (lii) D88487_2A8 [MICA/B]_H0_D88487_2A8 [MICA/B]_L0 (SEQ ID NOs: 647-654), (liii) D94837_3B12 [MICA/B]_H0_D94837_3B12 [MICA/B]_L0 (SEQ ID NOs: 655-662), (liv) D99122_1H7 [MICA/B]_H0_D99122_1H7 [MICA/B]_L0 (SEQ ID NOs: 663-670), (lv) D99136_2E8 [MICA/B]_H0_D99136_2E8 [MICA/B]_L0 (SEQ ID NOs: 671-678), (lvi) D88487_2A8 [MICA/B]_H0_D88487_2A8 [MICA/B]_L0 (SEQ ID NOs: 647-654), (lvii) 3F9[MICA/B]_H0_3F9[MICA/B]_L0 (SEQ ID NOs: 679-686), (lviii) 6E1[MICA/B]_H0_6E1[MICA/B]_L0 (SEQ ID NOs: 687-694), (lix) 7C6[MICA/B]_H0_7C6[MICA/B]_L0 (SEQ ID NOs: 695-702), (lx) 13A9 [MICA/B]_H0_13A9 [MICA/B]_L0 (SEQ ID NOs: 703-710), and (lxi) 1D5 [MICA/B]_H0_1D5 [MICA/B]_L0 (SEQ ID NOs: 711-718); and the αNKp46 is a scFv selected from the group including: (i) 2C10A3.372[NKp46]_H1_2C10A3.372[NKp46]_L1 (SEQ ID NOs: 719-726), and (ii) NKp46-A[NKp46]_H_NKp46-A[NKp46]_L (SEQ ID NOs: 727-734).

In particular 2+1 format embodiments, the αNKp46 ABD is the Fab and has the V_{H}_V_{L} pair selected from the group including: (i) 2C10A3.372[NKp46]_H1_2C10A3.372[NKp46]_L1 (SEQ ID NOs: 719-726), and (ii) NKp46-A[NKp46]_H_NKp46-A[NKp46]_L (SEQ ID NOs: 727-734); and the αMICA/B is a scFv selected from the group including: (i) D94837_1E11_1 [MICA/B]_H0_D94837_1E11_1 [MICA/B]_L0 (SEQ ID NOs: 239-246), (ii) D94837_1E11_1 [MICA/B]_H1_D94837_1E11_1 [MICA/B]_L1 (SEQ ID NOs: 247-254), (iii) D94837_1E11_1 [MICA/B]_H1_D94837_1E11_1 [MICA/B]_L2 (SEQ ID NOs: 255-262), (iv) D94837_1E11_1 [MICA/B]_H2_D94837_1E11_1 [MICA/B]_L1 (SEQ ID NOs: 263-270), (v) D94837_1E11_1 [MICA/B]_H2_D94837_1E11_1 [MICA/B]_L2 (SEQ ID NOs: 271-278), (vi) 2E5 [MICA/B]_H0_2E5 [MICA/B]_L0 (SEQ ID NOs: 279-286), (vii) 2E5 [MICA/B]_H1_2E5 [MICA/B]_L1 (SEQ ID NOs: 287-294), (viii) 2E5 [MICA/B]_H1_2E5 [MICA/B]_L2 (SEQ ID NOs: 295-302), (ix) 2E5 [MICA/B]_H2_2E5 [MICA/B]_L1 (SEQ ID NOs: 303-310), (x) 2E5 [MICA/B]_H2_2E5 [MICA/B]_L2 (SEQ ID NOs: 311-318), (xi) D94852_2E12 [MICA/B]_H0_D94852_2E12 [MICA/B]_L0 (SEQ ID NOs: 319-326), (xii) D94852_2E12 [MICA/B]_H1_D94852_2E12 [MICA/B]_L1 (SEQ ID NOs: 327-334), (xiii) D94852_2E12 [MICA/B]_H1_D94852_2E12 [MICA/B]_L2 (SEQ ID NOs: 335-342), (xiv) D94852_2E12 [MICA/B]_H2_D94852_2E12 [MICA/B]_L1 (SEQ ID NOs: 343-350), (xv) D94852_2E12 [MICA/B]_H2_D94852_2E12 [MICA/B]_L2 (SEQ ID NOs: 351-358), (xvi) D99136_2F7 [MICA/B]_H0_D99136_2F7 [MICA/B]_L0 (SEQ ID NOs: 359-366), (xvii) D99136_2F7 [MICA/B]_H1_D99136_2F7 [MICA/B]_L1 (SEQ ID NOs: 367-374), (xviii) D99136_2F7 [MICA/B]_H1_D99136_2F7 [MICA/B]_L2 (SEQ ID NOs: 375-382), (xix) D99136_2F7 [MICA/B]_H2_D99136_2F7 [MICA/B]_L1 (SEQ ID NOs: 383-390), (xx) D99136_2F7 [MICA/B]_H2_D99136_2F7 [MICA/B]_L2 (SEQ ID NOs: 391-398), (xxi) D103388_1C7 [MICA/B]_H0_D103388_1C7 [MICA/B]_L0 (SEQ ID NOs: 399-406), (xxii) D103388_1C7 [MICA/B]_H1_D103388_1C7 [MICA/B]_L1 (SEQ ID NOs: 407-414), (xxiii) D103388_1C7 [MICA/B]_H1_D103388_1C7 [MICA/B]_L2 (SEQ ID NOs: 415-422), (xxiv) D103388_1C7 [MICA/B]_H2_D103388_1C7 [MICA/B]_L1 (SEQ ID NOs: 423-430), (xxv) D103388_1C7 [MICA/B]_H2_D103388_1C7 [MICA/B]_L2 (SEQ ID NOs: 431-438), (xxvi) D103388_1D7 [MICA/B]_H0_D103388_1D7 [MICA/B]_L0 (SEQ ID NOs: 439-446), (xxvii) D103388_1D7 [MICA/B]_H1_D103388_1D7 [MICA/B]_L1 (SEQ ID NOs: 447-454), (xxviii) D103388_1D7 [MICA/B]_H1_D103388_1D7 [MICA/B]_L2 (SEQ ID NOs: 455-462), (xxix) D103388_1D7 [MICA/B]_H2_D103388_1D7 [MICA/B]_L1 (SEQ ID NOs: 463-470), (xxx) D103388_1D7 [MICA/B]_H2_D103388_1D7 [MICA/B]_L2 (SEQ ID NOs: 471-478), (xxxi) D105317_1A2 [MICA/B]_H0_D105317_1A2 [MICA/B]_L0 (SEQ ID NOs: 479-486), (xxxii) D105317_1A2 [MICA/B]_H1_D105317_1A2 [MICA/B]_L1 (SEQ ID NOs: 487-494), (xxxiii) D105317_1A2 [MICA/B]_H1_D105317_1A2 [MICA/B]_L2 (SEQ ID NOs: 495-502), (xxxiv) D105317_1A2 [MICA/B]_H2_D105317_1A2 [MICA/B]_L1 (SEQ ID NOs: 503-510), (xxxv) D105317_1A2 [MICA/B]_H2_D105317_1A2 [MICA/B]_L2 (SEQ ID NOs: 511-518), (xxxvi) D99136_2C11 [MICA/B]_H0_D99136_2C11 [MICA/B]_L0 (SEQ ID NOs: 519-526), (xxxvii) D99136_2C11 [MICA/B]_H1_D99136_2C11 [MICA/B]_L1 (SEQ ID NOs: 527-534), (xxxviii) D99136_2C11 [MICA/B]_H1_D99136_2C11 [MICA/B]_L2 (SEQ ID NOs: 535-542), (xxxix) D99136_2C11 [MICA/B]_H2_D99136_2C11 [MICA/B]_L1 (SEQ ID NOs: 543-550), (xl) D99136_2C11 [MICA/B]_H2_D99136_2C11 [MICA/B]_L2 (SEQ ID NOs: 551-558), (xli) D103388_1B11-2 [MICA/B]_H0_D103388_1B11-2 [MICA/B]_L0 (SEQ ID NOs: 559-566), (xlii) D105317_1B6 [MICA/B]_H0_D105317_1B6 [MICA/B]_L0 (SEQ ID NOs: 567-574), (xliii) D105317_1C8 [MICA/B]_H0_D105317_1C8 [MICA/B]_L0 (SEQ ID NOs: 575-582), (xliv) D105317_1F6 [MICA/B]_H0_D105317_1F6 [MICA/B]_L0 (SEQ ID NOs: 583-590), (xlv) D105317_1F7 [MICA/B]_H0_D105317_1F7 [MICA/B]_L0 (SEQ ID NOs: 591-598), (xlvi) D94837_1D3 [MICA/B]_H0_D94837_1D3 [MICA/B]_L0 (SEQ ID NOs: 599-606), (xlvii) D94837_1D8 [MICA/B]_H0_D94837_1D8 [MICA/B]_L0 (SEQ ID NOs: 607-614), (xlviii) D94837_1D9 [MICA/B]_H0_D94837_1D9 [MICA/B]_L0 (SEQ ID NOs: 615-622), (xlix) D94837_1E1 [MICA/B]_H0_D94837_1E1 [MICA/B]_L0 (SEQ ID NOs: 623-630), (1) D94852_2D4 [MICA/B]_H0_D94852_2D4 [MICA/B]_L0 (SEQ ID NOs: 631-638), (li) D94852_2G8 [MICA/B]_H0_D94852_2G8 [MICA/B]_L0 (SEQ ID NOs: 639-646), (lii) D88487_2A8 [MICA/B]_H0_D88487_2A8 [MICA/B]_L0 (SEQ ID NOs: 647-654), (liii) D94837_3B12 [MICA/B]_H0_D94837_3B12 [MICA/B]_L0 (SEQ ID NOs: 655-662), (liv) D99122_1H7 [MICA/B]_H0_D99122_1H7 [MICA/B]_L0 (SEQ ID NOs: 663-670), (lv) D99136_2E8 [MICA/B]_H0_D99136_2E8 [MICA/B]_L0 (SEQ ID NOs: 671-678), (lvi) D88487_2A8 [MICA/B]_H0_D88487_2A8 [MICA/B]_L0 (SEQ ID NOs: 647-654), (lvii) 3F9[MICA/B]_H0_3F9[MICA/B]_L0 (SEQ ID NOs: 679-686), (lviii) 6E1[MICA/B]_H0_6E1[MICA/B]_L0 (SEQ ID NOs: 687-694), (lix) 7C6[MICA/B]_H0_7C6[MICA/B]_L0 (SEQ ID NOs: 695-702), (lx) 13A9 [MICA/B]_H0_13A9 [MICA/B]_L0 (SEQ ID NOs: 703-710), and (lxi) 1D5 [MICA/B]_H0_1D5 [MICA/B]_L0 (SEQ ID NOs: 711-718).

In particular 2+1 format embodiments, the αMICA/B ABD is the Fab and has the V_{H}_V_{L} pair selected from the group including: (i) D94837_1E11_1 [MICA/B]_H0_D94837_1E11_1 [MICA/B]_L0 (SEQ ID NOs: 239-246), (ii) D94837_1E11_1 [MICA/B]_H1_D94837_1E11_1 [MICA/B]_L1 (SEQ ID NOs: 247-254), (iii) D94837_1E11_1 [MICA/B]_H1_D94837_1E11_1 [MICA/B]_L2 (SEQ ID NOs: 255-262), (iv) D94837_1E11_1 [MICA/B]_H2_D94837_1E11_1 [MICA/B]_L1 (SEQ ID NOs: 263-270), (v) D94837_1E11_1 [MICA/B]_H2_D94837_1E11_1 [MICA/B]_L2 (SEQ ID NOs: 271-278), (vi) 2E5 [MICA/B]_H0_2E5 [MICA/B]_L0 (SEQ ID NOs: 279-286), (vii) 2E5 [MICA/B]_H1_2E5 [MICA/B]_L1 (SEQ ID NOs: 287-294), (viii) 2E5 [MICA/B]_H1_2E5 [MICA/B]_L2 (SEQ ID NOs: 295-302), (ix) 2E5 [MICA/B]_H2_2E5 [MICA/B]_L1 (SEQ ID NOs: 303-310), (x) 2E5 [MICA/B]_H2_2E5 [MICA/B]_L2 (SEQ ID NOs: 311-318), (xi) D94852_2E12 [MICA/B]_H0_D94852_2E12 [MICA/B]_L0 (SEQ ID NOs: 319-326), (xii) D94852_2E12 [MICA/B]_H1_D94852_2E12 [MICA/B]_L1 (SEQ ID NOs: 327-334), (xiii) D94852_2E12 [MICA/B]_H1_D94852_2E12 [MICA/B]_L2 (SEQ ID NOs: 335-342), (xiv) D94852_2E12 [MICA/B]_H2_D94852_2E12 [MICA/B]_L1 (SEQ ID NOs: 343-350), (xv) D94852_2E12 [MICA/B]_H2_D94852_2E12 [MICA/B]_L2 (SEQ ID NOs: 351-358), (xvi) D99136_2F7 [MICA/B]_H0_D99136_2F7 [MICA/B]_L0 (SEQ ID NOs: 359-366), (xvii) D99136_2F7 [MICA/B]_H1_D99136_2F7 [MICA/B]_L1 (SEQ ID NOs: 367-374), (xviii) D99136_2F7 [MICA/B]_H1_D99136_2F7 [MICA/B]_L2 (SEQ ID NOs: 375-382), (xix) D99136_2F7 [MICA/B]_H2_D99136_2F7 [MICA/B]_L1 (SEQ ID NOs: 383-390), (xx) D99136_2F7 [MICA/B]_H2_D99136_2F7 [MICA/B]_L2 (SEQ ID NOs: 391-398), (xxi) D103388_1C7 [MICA/B]_H0_D103388_1C7 [MICA/B]_L0 (SEQ ID NOs: 399-406), (xxii) D103388_1C7 [MICA/B]_H1_D103388_1C7 [MICA/B]_L1 (SEQ ID NOs: 407-414), (xxiii) D103388_1C7 [MICA/B]_H1_D103388_1C7 [MICA/B]_L2 (SEQ ID NOs: 415-422), (xxiv) D103388_1C7 [MICA/B]_H2_D103388_1C7 [MICA/B]_L1 (SEQ ID NOs: 423-430), (xxv) D103388_1C7 [MICA/B]_H2_D103388_1C7 [MICA/B]_L2 (SEQ ID NOs: 431-438), (xxvi) D103388_1D7 [MICA/B]_H0_D103388_1D7 [MICA/B]_L0 (SEQ ID NOs: 439-446), (xxvii) D103388_1D7 [MICA/B]_H1_D103388_1D7 [MICA/B]_L1 (SEQ ID NOs: 447-454), (xxviii) D103388_1D7 [MICA/B]_H1_D103388_1D7 [MICA/B]_L2 (SEQ ID NOs: 455-462), (xxix) D103388_1D7 [MICA/B]_H2_D103388_1D7 [MICA/B]_L1 (SEQ ID NOs: 463-470), (xxx) D103388_1D7 [MICA/B]_H2_D103388_1D7 [MICA/B]_L2 (SEQ ID NOs: 471-478), (xxxi) D105317_1A2 [MICA/B]_H0_D105317_1A2 [MICA/B]_L0 (SEQ ID NOs: 479-486), (xxxii) D105317_1A2 [MICA/B]_H1_D105317_1A2 [MICA/B]_L1 (SEQ ID NOs: 487-494), (xxxiii) D105317_1A2 [MICA/B]_H1_D105317_1A2 [MICA/B]_L2 (SEQ ID NOs: 495-502), (xxxiv) D105317_1A2 [MICA/B]_H2_D105317_1A2 [MICA/B]_L1 (SEQ ID NOs: 503-510), (xxxv) D105317_1A2 [MICA/B]_H2_D105317_1A2 [MICA/B]_L2 (SEQ ID NOs: 511-518), (xxxvi) D99136_2C11 [MICA/B]_H0_D99136_2C11 [MICA/B]_L0 (SEQ ID NOs: 519-526), (xxxvii) D99136_2C11 [MICA/B]_H1_D99136_2C11 [MICA/B]_L1 (SEQ ID NOs: 527-534), (xxxviii) D99136_2C11 [MICA/B]_H1_D99136_2C11 [MICA/B]_L2 (SEQ ID NOs: 535-542), (xxxix) D99136_2C11 [MICA/B]_H2_D99136_2C11 [MICA/B]_L1 (SEQ ID NOs: 543-550), (xl) D99136_2C11 [MICA/B]_H2_D99136_2C11 [MICA/B]_L2 (SEQ ID NOs: 551-558), (xli) D103388_1B11-2 [MICA/B]_H0_D103388_1B11-2 [MICA/B]_L0 (SEQ ID NOs: 559-566), (xlii) D105317_1B6 [MICA/B]_H0_D105317_1B6 [MICA/B]_L0 (SEQ ID NOs: 567-574), (xliii) D105317_1C8 [MICA/B]_H0_D105317_1C8 [MICA/B]_L0 (SEQ ID NOs: 575-582), (xliv) D105317_1F6 [MICA/B]_H0_D105317_1F6 [MICA/B]_L0 (SEQ ID NOs: 583-590), (xlv) D105317_1F7 [MICA/B]_H0_D105317_1F7 [MICA/B]_L0 (SEQ ID NOs: 591-598), (xlvi) D94837_1D3 [MICA/B]_H0_D94837_1D3 [MICA/B]_L0 (SEQ ID NOs: 599-606), (xlvii) D94837_1D8 [MICA/B]_H0_D94837_1D8 [MICA/B]_L0 (SEQ ID NOs: 607-614), (xlviii) D94837_1D9 [MICA/B]_H0_D94837_1D9 [MICA/B]_L0 (SEQ ID NOs: 615-622), (xlix) D94837_1E1 [MICA/B]_H0_D94837_1E1 [MICA/B]_L0 (SEQ ID NOs: 623-630), (1) D94852_2D4 [MICA/B]_H0_D94852_2D4 [MICA/B]_L0 (SEQ ID NOs: 631-638), (li) D94852_2G8 [MICA/B]_H0_D94852_2G8 [MICA/B]_L0 (SEQ ID NOs: 639-646), (lii) D88487_2A8 [MICA/B]_H0_D88487_2A8 [MICA/B]_L0 (SEQ ID NOs: 647-654), (liii) D94837_3B12 [MICA/B]_H0_D94837_3B12 [MICA/B]_L0 (SEQ ID NOs: 655-662), (liv) D99122_1H7 [MICA/B]_H0_D99122_1H7 [MICA/B]_L0 (SEQ ID NOs: 663-670), (lv) D99136_2E8 [MICA/B]_H0_D99136_2E8 [MICA/B]_L0 (SEQ ID NOs: 671-678), (lvi) D88487_2A8 [MICA/B]_H0_D88487_2A8 [MICA/B]_L0 (SEQ ID NOs: 647-654), (lvii) 3F9[MICA/B]_H0_3F9[MICA/B]_L0 (SEQ ID NOs: 679-686), (lviii) 6E1[MICA/B]_H0_6E1[MICA/B]_L0 (SEQ ID NOs: 687-694), (lix) 7C6[MICA/B]_H0_7C6[MICA/B]_L0 (SEQ ID NOs: 695-702), (lx) 13A9 [MICA/B]_H0_13A9 [MICA/B]_L0 (SEQ ID NOs: 703-710), and (lxi) 1D5 [MICA/B]_H0_1D5 [MICA/B]_L0 (SEQ ID NOs: 711-718); and the αNKp46 is a scFv selected from the group including: (i) 2C10A3.372[NKp46]_H1_2C10A3.372[NKp46]_L1 (SEQ ID NOs: 719-726), and (ii) NKp46-A[NKp46]_H_NKp46-A[NKp46]_L (SEQ ID NOs: 727-734).

### J. Additional Embodiments

[Embodiment A] In one embodiment, the disclosure provides a heterodimeric antibody, comprising: (a) a first monomer; (b) a second monomer; and (c) a third monomer, wherein the first monomer, the second monomer, and the third monomer comprise: (i) the amino acid sequences of SEQ ID NOs: 859-861 of XENP44543, respectively, (ii) the amino acid sequences of SEQ ID NOs: 862-864 of XENP45903, respectively, (iii) the amino acid sequences of SEQ ID NOs: 865-867 of XENP45902, respectively, (iv) the amino acid sequences of SEQ ID NOs: 868-870 of XENP44549, respectively, (v) the amino acid sequences of SEQ ID NOs: 871-873 of XENP44551, respectively, (vi) the amino acid sequences of SEQ ID NOs: 874-876 of XENP44553, respectively, or (vii) the amino acid sequences of SEQ ID NOs: 877-879 of XENP44552, respectively, as depicted in FIG. 71.

[Embodiment B] In another embodiment, the disclosure provides a nucleic acid composition comprising nucleic acids encoding the first monomer, the second monomer, and the third monomer of any one of the heterodimeric antibodies of Embodiment A.

[Embodiment C] In another embodiment, the disclosure provides an expression vector comprising the nucleic acid(s) of Embodiment B.

[Embodiment D] In another embodiment, the disclosure provides a host cell transformed with the expression vector of Embodiment C.

[Embodiment E] In another embodiment, the disclosure provides a method of making a heterodimeric antibody, comprising: (a) culturing the host cell of Embodiment D under conditions wherein the heterodimeric antibody is expressed; and (b) recovering the heterodimeric antibody.

[Embodiment F] In another embodiment, the disclosure provides a method of treating cancer, comprising: administering to a human subject an antibody comprising any one of the heterodimeric antibodies of Embodiment A.

[Embodiment G] In another embodiment, the disclosure provides a heterodimeric antibody, comprising: (a) a first monomer, comprising: (i) an anti-NKp46 scFv comprising a first variable heavy VH1 domain, an scFv linker, and a first variable light VL1 domain, and (ii) a first Fc domain, wherein the scFv is covalently attached to the N-terminus of the first Fc domain using a domain linker; (b) a second monomer comprising a VH2-CH1-hinge-CH2-CH3 monomer, wherein VH2 is a second variable heavy domain and CH2-CH3 is a second Fc domain; and (c) a light chain comprising a second variable light VL2 domain, wherein the second variable heavy VH2 domain and the second variable light VL2 domain form a B7H3 antigen binding domain.

[Embodiment H] In another embodiment, the disclosure provides the heterodimeric antibody of Embodiment G, wherein the first monomer, the second monomer, and the light chain of the heterodimeric antibody are selected from the group including: (i) the amino acid sequences of SEQ ID NOs: 859-861 of XENP44543, respectively, (ii) the amino acid sequences of SEQ ID NOs: 862-864 of XENP45903, respectively, (iii) the amino acid sequences of SEQ ID NOs: 865-867 of XENP45902, respectively, (iv) the amino acid sequences of SEQ ID NOs: 868-870 of XENP44549, respectively, (v) the amino acid sequences of SEQ ID NOs: 871-873 of XENP44551, respectively, (vi) the amino acid sequences of SEQ ID NOs: 874-876 of XENP44553, respectively, and (vii) the amino acid sequences of SEQ ID NOs: 877-879 of XENP44552, respectively, as depicted in FIG. 71.

[EMBODIMENT I] In another embodiment, the disclosure provides a method of treating cancer, comprising: administering to a human subject an antibody comprising: (i) a MICA/B antigen binding domain as described herein, (ii) a NKp46 antigen binding domain as described herein, or (iii) a heterodimeric antibody as described herein, to the human subject.

[EMBODIMENT J] In another embodiment and in accordance with Embodiment I, the disclosure provides a method further comprising administering an IL-15-Fc fusion protein to the human subject.

[EMBODIMENT K] In another embodiment and in accordance with Embodiment K, the disclosure provides a method further comprising administering an IL-15-Fc fusion protein to the human subject, wherein the IL-15-Fc fusion protein comprises the amino acid sequences of SEQ ID NOs: 833-834 of XENP24045.

[EMBODIMENT L] In another embodiment, the disclosure provides a method of blocking MICA/B shedding by NK cells in a human subject, the method comprising: administering to the human subject a therapeutically effective amount of an antibody comprising: (i) a MICA/B antigen binding domain as described herein, or (ii) a heterodimeric antibody as described herein.

[EMBODIMENT M] In another embodiment and in accordance with Embodiment L, the disclosure provides a method wherein CD16 and/or NKG2D is activated on the NK cells.

[EMBODIMENT N] In another embodiment and in accordance with Embodiment L or M, the disclosure provides a method further comprising administering an IL-15-Fc fusion protein to the human subject.

[EMBODIMENT O] In another embodiment and in accordance with Embodiment N, the disclosure provides a method further comprising administering an IL-15-Fc fusion protein to the human subject, wherein the IL-15-Fc fusion protein comprises the amino acid sequences of SEQ ID NOs: 833-834 of XENP24045.

### VI. Nucleic Acids

The disclosure further provides nucleic acid compositions encoding the anti-NKp46 antibodies provided herein, including, but not limited to, NKp46×MICA/B bispecific antibodies and NKp46 monospecific antibodies.

As will be appreciated by those in the art, the nucleic acid compositions will depend on the format and scaffold of the heterodimeric protein. Thus, for example, when the format requires three amino acid sequences, such as for the 1+1 Fab-scFv-Fc format (e.g., a first amino acid monomer comprising an Fc domain and a scFv, a second amino acid monomer comprising a heavy chain and a light chain), three nucleic acid sequences can be incorporated into one or more expression vectors for expression. Similarly, some formats (e.g., dual scFv formats) only two nucleic acids are needed; again, they can be put into one or two expression vectors.

As is known in the art, the nucleic acids encoding the components of the antibodies described herein can be incorporated into expression vectors as is known in the art, and depending on the host cells used to produce the heterodimeric antibodies described herein. Generally the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the antibodies described herein are then transformed into any number of different types of host cells as is well known in the art, including mammalian, bacterial, yeast, insect and/or fungal cells, with mammalian cells (e.g., CHO cells), finding use in many embodiments.

In some embodiments, nucleic acids encoding each monomer and the optional nucleic acid encoding a light chain, as applicable depending on the format, are each contained within a single expression vector, generally under different or the same promoter controls. In embodiments of particular use in the antibodies described herein, each of these two or three nucleic acids are contained on a different expression vector. As shown herein and in U.S. Pat. No. 9,822,186, hereby incorporated by reference, different vector ratios can be used to drive heterodimer formation. That is, surprisingly, while the proteins comprise first monomer:second monomer:light chains (in the case of many of the embodiments herein that have three polypeptides comprising the heterodimeric antibody) in a 1:1:2 ratio, these are not the ratios that give the best results.

The heterodimeric antibodies described herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (p1) of each monomer so that such that each monomer has a different pI and the heterodimer also has a distinct p1, thus facilitating isoelectric purification of the “1+1 Fab-scFv-Fc” and “2+1” heterodimers (e.g., anionic exchange columns, cationic exchange columns). These substitutions also aid in the determination and monitoring of any contaminating dual scFv-Fc and mAb homodimers post-purification (e.g., IEF gels, clEF, and analytical IEX columns).

Additionally, as is known in the art, N- and/or C-terminal clipping can occur during protein synthesis, whereby the heavy chains depicted herein may have the C-terminal lysine (K447) removed, as well as the penultimate glycine (G446), and optionally additional amino acids residues (e.g., from 1, 2, 3, 4, 5, 6, 7, 8, 9, 10 or more C-terminal amino acids can be removed). Alternatively, the nucleic acids encoding the heavy chains of the bispecific antibodies can be engineered such that these terminal residues are eliminated entirely to facilitate additional homogeneity.

### VII. Biological and Biochemical Functionality of the Heterodimeric Bispecific Antibodies

Generally, the bispecific NKp46×MICA/B antibodies described herein are administered to patients with cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### VIII. Treatments

Once made, the compositions of the invention find use in a number of oncology applications, by treating cancer, generally by enhancing immune responses, including, activating NK cells, enhancing NK cell mediated lysis of tumor cells. Such compositions can be combined with proinflammatory cytokines for increased cytotoxicity against tumor cells.

### A. Combination with Interleukin 15 Therapies

In some embodiments, a bispecific anti-NKp46×anti-MICA/B antibody described herein can be used in combination with an IL-15 therapy. In some embodiments, the IL-15 therapy includes, but is not limited to, an IL-15-Fc based therapy.

Non-limiting examples of an IL-15 therapy include administration to a human subject an IL-15 protein or a fragment thereof, an IL-15 variant protein or a fragment thereof, an IL-15 fusion protein, or an IL-15 agonist. Useful IL-15 fusion proteins include, but are not limited to those shown in FIG. 66, such as, for example, XENP24045. Sequences of exemplary IL-15 fusion proteins are provided in the sequence listing, such as, for example, SEQ ID NOs: 833-834, as shown in FIG. 66.

IL-15 agonists are well-known in the art. Non-limiting examples of IL-15 agonists include PF-07209960 (Pfizer), KD033 (Kadmon), SOT201/SO-C108 (SOTIO/Cytune), SOT101/SO C101/RLI-15 (SOTIO/Cytune), XmAb24306 (Genentech/Xencor), NKTR-255 (Nektar), ALT-803/N-803/Anktiva (Altor Bioscience), and NIZ985 (Novartis). In some embodiments, the IL-15 agonist is an IL-15 protein. The wild-type human IL-15 protein comprises the amino acid sequence shown in FIG. 22C (see, e.g., SEQ ID NOs: 1079 and 1080) of U.S. Publication No. 2023/0151095, which is incorporated herein by reference in its entirety. In some embodiments, the IL-15 agonist is an IL-15 variant protein. IL-15 variant proteins are well-known in the art. See, e.g., U.S. Pat. Nos. 5,552,303; 5,574,138; 6,001,973; 6,013,480; 6,548,065; 6,764,836; 6,998,476; 7,858,081; 8,163,879; 8,178,660; 8,415,456; 8,507,222; 8,940,288; 9,303,080; 9,365,630; 9,371,368; 9,428,563; 9,428,573; 9,493,533; 9,725,492; 9,790,261; 9,932,387; and 9,975,937; U.S. Publication Nos. 2004/009149, 2006/0057680, 2006/0236411, 2006/0257361, 2007/0106066, 2007/0134718, 2009/0105455, 2009/0238791, 2015/0359853, 2016/0130318, 2016/0175459, 2016/0184399, 2016/0275236, 2017/0020963, 2017/0202924, 2017/0246253, 2018/0044424, 2018/0126003, and 2018/0200366; and PCT Publication Nos. WO9527722, WO2016095642, WO2017081082, WO2017046200, WO2017136818, WO2018013855 WO2018023093, WO2018071918, and WO2018071919, each of which is incorporated herein by reference in its entirety. Sequences of human IL-15 proteins, such as the precursor protein and the mature protein are set for in FIG. 22C as SEQ ID NOS:1079 and 1080 in U.S. Publication No. 2023/0151095. Further, sequences for: human IL-15Rα, the extracellular domain of human IL-15Rα, human IL-15Rα sushi domain, human IL-15Rβ, and the extracellular domain of human IL-15Rβ are set forth in FIGS. 22C-22D of U.S. Publication No. 2023/0151095 as SEQ ID NOs: 1081, 1082, 1083, 1084, and 1085, respectively, each of which is incorporated by reference in their entirety.

In some instances, administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

### B. Antibody Compositions for In Vivo Administration

Formulations of the antibodies used in accordance with the antibodies described herein are prepared for storage by mixing an antibody having the desired degree of purity with optional pharmaceutically acceptable carriers, excipients, or stabilizers (Remington's Pharmaceutical Sciences 16th edition, Osol, A. Ed. [1980]), in the form of lyophilized formulations or aqueous solutions.

### C. Administration Modalities

The antibodies provided herein administered to a subject, in accord with known methods, such as intravenous administration as a bolus or by continuous infusion over a period of time.

### D. Treatment Modalities

In the methods described herein, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MM) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the disclosure includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for cancer therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the disclosure are dictated by and directly dependent on (a) the unique characteristics of the active compound and the particular therapeutic effect to be achieved, and (b) the limitations inherent in the art of compounding such an active compound for the treatment of sensitivity in individuals.

All cited references are herein expressly incorporated by reference in their entirety.

Whereas particular embodiments of the disclosure have been described above for purposes of illustration, it will be appreciated by those skilled in the art that numerous variations of the details may be made without departing from the invention as described in the appended claims.

### EXAMPLES

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### A. Example 1: MICA/B Binding Domains

Tumor microenvironment induced antigens present a unique opportunity to effectively target diseased tissue over normal and to modulate the immune suppression they might elicit. MICA and MICB (MICA/B) are stress-induced antigens expressed in a range of cancers. As depicted in FIG. 47, pan tumor TMA was stained with MICA/B antibodies showing that breast, testis, esophagus, and stomach show tumor MICA/B expression (pancreas, skin, lung, and ovary are additional tumor histologies showing tumor MICA/B expression, not shown). MICA/B antigens are recognized by NKG2D, an activating receptor on NK and CD8+ T cells. While the membrane-bound form of MICA/B is immuno-stimulatory, the cleaved soluble form found in the tumor microenvironment prevents NKG2D mediated tumor cell recognition. To stop tumor escape and, concurrently, stimulate the innate and adaptive immune responses, antibodies targeting MICA/B were developed. Anti-MICA/B antibodies block proteolytic cleavage, increase MICA/B membrane surface densities, and activate NK and CD8+ T cells by bringing membrane bound MICA/B to NKG2D. In summary, MICA/B mechanisms of action include ADCC, NKG2D agonism, and blockade of MICA and MICB cleavage, as depicted in FIG. 48. Prior to developing novel MICA/B binding domains, these mechanisms were investigated using a prior art MICA/B antibody.

NK cells were co-cultured with A375-B2M-KO-RP tumor cell line and dose titration of MICA/B mAb alone, MICA/B mAb with blocking NKG2D mAb, RSV isotype control mAb alone, or RSV mAb with blocking NKG2D mAb. Target lysis and IFNγ secretion are depicted in FIG. 49, demonstrating ADCC and NKG2D agonism.

CHO-MICA cells and CHO-MICB cells were incubated with MICA/B mAb, RSV mAb, or batimastat (an inhibitor of cell surface proteases). Soluble MICA and MICB in the supernatant were measured by ELISA as depicted in FIG. 50, demonstrating blockade of sMICA and sMICB.

### 1A. Novel MICA/B Binding Domains which Block MICA/B Shedding and Bind Multiple Alleles

As depicted in FIG. 20, soluble MICA/B (sMICA/B) bind NKG2D receptor on NK cells inhibiting signaling. Additionally, there are numerous MICA and MICB alleles, sequences for illustrative alleles are depicted in FIGS. 1 and 2. Population frequencies of MICA and MICB allelic variants are depicted in FIG. 51. Accordingly, novel MICA/B binding domains were generated in a campaign using single cell technology to identify MICA/B binding domains that: a) are capable of binding to cell surface MICA/B and prevent shedding; and b) are able to bind multiple alleles. Alignment of α3 domain sequences for MICA and MICB variants with a high population frequency is depicted in FIG. 52, to identify higher frequency MICA and MICB variants. Five BALB/c and five C57BL/6J mice were immunized with MICA*002, MICA*004, MICA*008, MICB*004, and MICB*005 to raise antibodies specific for multiple MICA and MICB alleles. Injections were administered in the hock and base of neck, twice weekly for four weeks, with an additional boost 5 days before harvest. Antibody secreting plasma cells were isolated and enriched from lymph nodes harvested from immunized mice.

Bulk plasma cells were imported onto a microfluidic chip and single cells were sequestered into individual wells for screening. Each plasma cell was individually screened for secretion of antigen-specific IgG that was detected using an antigen coated bead-based fluorescence assay on the Beacon. Individual cells of interest were lysed, their mRNA captured and used to generate cDNA encoding antibody VH and VL sequences. Sequences for such illustrative MICA/B binding domains (original murine sequences; and sequences humanized using string content optimization as described in see, e.g., U.S. Pat. No. 7,657,380, issued Feb. 2, 2010) are depicted in FIG. 21. All humanized variants of MICA/B binding domains 1E11-1 and 2C11 (designated as H1L1, H1L2, H2L1, and H2L2) maintained comparable binding to the parental murine H0L0 clones as depicted in FIG. 73.

### 1A(a). Characterizing Novel MICA/B Binding Domain

In order to begin characterization, the 277 hits obtained from the Beacon screening were cloned into IgG1 WT backbones for a small scale 5 mL transfection. Anti-MICA/B antibodies that did not express well (<200 μg/mL) were removed from consideration, and the remaining 164 antibodies were captured onto anti-human Fc capture chips, and screened against human MICA, cyno MICA, and human MICB using the Carterra® LSA platform. Out of the 164 antibodies screened, 48 clones showed binding to at least one of the antigens and progressed to further characterization. The KD data for select anti-MICA/B clones is depicted in FIG. 72. These 48 clones were also analyzed via ELISA using the DuoSet® ELISA development system for Human MICA (R&D cat #DY1300). Anti-MICA/B antibodies (at a final concentration of 30 ug/ml) were mixed with CHO engineered to express GFP-tagged MICA*004, and then an ELISA plate was coated with anti-MICA antibody and processed according to ELISA kit instructions (R&D cat #DY1300). The ability of the anti-MICA/B antibodies to bind MICA/B and their ability to block shedding were measured, and resulting data showed they are correlated. The levels of soluble MICA detected in the assay and the correlated inhibition of shedding and are depicted in FIG. 72.

Epitope binning was performed on the novel MICA/B binding domains and additional MICA/B binding domains as described below in 1B (formatted as bivalent mAbs with WT Fc), data for which are depicted in FIG. 36.

Next, the ability of the novel MICA/B binding domains to bind to cell surface MICA/B and prevent shedding was investigated further. Surface MICA/B upregulation inversely correlates with soluble MICA/B; therefore, surface MICA/B was used as a readout for screening the novel MICA/B binding domains for their ability to prevent MICA and MICB shedding (see FIG. 55). CHO cells were engineered to express various GFP-tagged MICA or MICB alleles and incubated with dose titration of the MICA/B binding domains for 24 or 48 hours. After 24 or 48 hours, supernatant was collected to determined GFP intensity data by Incucyte and analyze the soluble MIC levels. Area under curve (AUC) from the resulting dose responses are plotted, data for which are depicted in FIGS. 37, 43, and 63 showing that the novel MICA/B binding domains were able to bind/block MICA*004 shedding. Additionally, a similar experiment was performed using A375 cell engineered to over-express MICA*004, data for which are depicted in FIG. 44.

In similar experiments, the ability of the novel MICA/B binding domains to bind multiple MICA and MICB alleles was investigated. Data as depicted in FIGS. 38 and 39 show that that the novel MICA/B binding domains were able to bind and prevent shedding of multiple MICA and MICB allelic variants. Additionally, data as depicted in FIGS. 56 and 57 show that the MICA/B binding domains were able to bind both MICA and MICB.

Next, the ability of the novel MICA/B binding domains to bind and prevent shedding of MICA under normoxic versus hypoxic conditions was investigated, data for which are depicted in FIGS. 40, 45, and 46.

Finally, biological activity of the novel MICA/B binding domains were investigated. NK cells (purified from PBMC using EasySep™ Human NK Cell Isolation Kit from Stemcell Technologies) were incubated with MCF7-RFP target cells at indicated effector:target ratios and incubated with dose titration of the novel MICA/B binding domains (formatted as bivalent mAbs). Data depicting target cell lysis are depicted in FIG. 41; and data depicting IFNγ production by NK cells are depicted in FIG. 42. Data depicting correlation between IFNγ AUC and target lysis EC50 of different MICA/B mAbs are depicted in FIG. 53 to screen for mAbs that enable high IFNγ AUC at low EC50. FIG. 54 depicts data corresponding to just 1E11-1. Taken together, the data showed that 1E11-1, 2C11, and 1C7 stood out as some of the clones best able to bind multiple allelic variants of MICA and MICB.

### 1B. Additional MICA/B Binding Domains

Sequences for additional MICA/B binding domains known in the art are depicted in FIG. 22 and as SEQ ID NOS: 679-718.

### B. Example 2: NKp46 Binding Domains

### 2A. Novel NKp46 Binding Domains

NKp46 is an activating receptor on NK cells. Novel NKp46 binding domains were generated using in-house de novo phage libraries. Sequences for illustrative such NKp46 binding domains are depicted in FIG. 23.

### 2B. Additional NKp46 Binding Domains

Sequences for additional NKp46 binding domains are depicted in FIG. 24 and as SEQ ID NOS: 727-734. Additionally, NKp46 binding domains are utilized in NKp46×B7H3 bsAbs as described in Example 3 and depicted in FIG. 71.

### C. Example 3: B7H3 Binding Domains

The B7H3 binding domains used herein, and methods of their discovery have been previously described in U.S. patent application Ser. No. 17/407,135, hereby incorporated by reference. A B7H3 binding domains designated 6A1 utilized herein was obtained from rabbit hybridoma and humanized using string content optimization (see, e.g., U.S. Pat. No. 7,657,380, issued Feb. 2, 2010). Exemplary bivalent antibodies comprising the 6A1 binding domain are depicted in FIG. 71.

### D. Example 4: Engineering NKp46×MICA/B bsAbs and NKp46×B7H3 bsAbs

A number of formats for NKp46×MICA/B bsAbs were conceived, illustrative formats for which are outlined below and in FIG. 25.

### 4A(i). 1+1 Fab×scFv Format

One format utilizing a Fab domain and an scFv is the 1+1 Fab×scFv format (depicted schematically in FIG. 25A) which comprises a first monomer comprising a single-chain Fv (“scFv”) with a first antigen binding specificity covalently attached to a first heterodimeric Fc domain, a second monomer comprising a heavy chain variable region (VH) covalently attached to a complementary second heterodimeric Fc domain, and a light chain (LC) transfected separately so that a Fab domain having a second antigen binding specificity is formed with the variable heavy domain. Sequences for illustrative NKp46×MICA/B bsAbs (based on binding domains as described in Examples 1 and 2) in the 1+1 Fab×scFv format are depicted in FIG. 31. Additionally, sequences for illustrative NKp46×B7H3 bsAbs are depicted in FIG. 71.

### 4A(ii). 1+1 Empty×Fab-scFv Format

Another format utilizing a Fab domain and an scFv is the 1+1 empty×Fab-scFv format (depicted schematically in FIG. 25B) which comprises a first monomer comprising a first heterodimeric Fc domain, a second monomer comprising a heavy chain variable region (VH) covalently attached to an scFv further covalently attached to a complementary second heterodimeric Fc domain, and a light chain (LC) transfected separately so that a Fab domain having a second antigen binding specificity is formed with the variable heavy domain. Sequences for illustrative NKp46×MICA/B bsAbs (based on binding domains as described in Examples 1 and 2) in the 1+1 empty×Fab-scFv format are depicted in FIG. 32.

### 4A(iii). 2+1 Fab×Fab-scFv Format

A format utilizing two Fab domains and an scFv is the 2+1 Fab×Fab-scFv (depicted schematically in FIG. 25C) which comprises a first monomer comprising a VH domain covalently attached to an scFv (having a first antigen binding specificity) covalently attached to a first heterodimeric Fc domain, a second monomer comprising a VH domain covalently attached to a complementary second heterodimeric Fc domain, and a LC transfected separately so that Fab domains having a second antigen binding specificity are formed with the VH domains. Sequences for illustrative NKp46×MICA/B bsAbs (based on binding domains as described in Examples 1 and 2) in the 2+1 Fab×Fab-scFv format are depicted in FIG. 33.

### 4A(iv). 2+1 Fab2×scFv Format

A format utilizing two Fab domains and an scFv is the 2+1 Fab2×scFv (depicted schematically in FIG. 25D) which comprises a first monomer comprising an scFv (having a first antigen binding specificity) covalently attached to a first heterodimeric Fc domain, a second monomer comprising a VH domain covalently attached to another VH domain that is covalently attached to a complementary second heterodimeric Fc domain, and a LC transfected separately so that Fab domains having a second antigen binding specificity are formed with the VH domains. Sequences for illustrative NKp46×MICA/B bsAbs (based on binding domains as described in Examples 1 and 2) in the 2+1 Fab2×scFv format are depicted in FIG. 34.

### 4A(v). 2+1 mAb-scFv Format

An additional format utilizing two Fab domains and scFv is the 2+1 mAb-scFv format (depicted schematically in FIG. 25E) which comprises a first monomer comprising a VH domain covalently attached to a first heterodimeric Fc domain covalently attached to an scFv (having a first antigen binding specificity), a second monomer comprising a VH domain covalently attached to a complementary second heterodimeric Fc domain, and a LC transfected separately so that Fab domains having a second antigen specificity are formed with the VH domains. Sequences for illustrative NKp46×MICA/B bsAbs (based on binding domains as described in Examples 1 and 2) in the 2+1 mAb-scFv format are depicted in FIG. 35.

### E. Example 5: ADCC-Enhanced Fc Engineering

As depicted in FIG. 27, one hypothesized mechanism of action for MICA/B bsAbs is that 1) the bsAb engages MICA/B and tumor antigen on cancer cells, 2) the bsAb engages and activates CD16 on NK cells, and 3) the MICA/B bound by the bsAb engages and signals via NKG2D on NK cells. Accordingly, the antibodies (mAbs and bsAbs) of the invention were engineered with Fc having WT CD16 binding or enhanced CD16 binding. Fc variants enabling enhanced CD16 binding (or ADCC-enhanced variants) include those in FIG. 7. Antibodies were also engineered with FcKO variants PVA_/S267K abrogating CD16 binding to compare with antibodies retaining WT or enhanced CD16 binding.

ADCC enhancing S239D/I332E Fc variants in antibody constructs can result in decreased stability and a lower yield in production. To address this, B7H3 antibodies in the 1+1 Fab-scFv-Fc format (sequences for which are depicted in FIG. 28) were engineered and produced with Fc regions having different combinations of S239D and/or 1332E substitutions on either one or both Fc monomers, either symmetrically or asymmetrically. B7H3 was used as the ABD in both the Fab and the scFv arm for these test articles so that the binding and activity assays would be solely assessing CD16 engagement and not be impacted by MICA/B binding. FIGS. 14 and 15 depict the sequences of the various symmetric & asymmetric ADCC-enhanced backbone sequences, both without and with the addition of the Xtend M428L/N434S variants. In order to investigate the ADCC activity, MCF7 cells were seeded in a 96 well plate and incubated overnight. The next day, the ADCC Reporter Bioassay kit from Promega (Catalog #G7018) was used according to the manufacturer's protocol. Test articles and effector cells were added to the MCF7 cells, and after a 6 hour incubation, Bio-Glo luciferase reagent was added and plates were analyzed with an EnVision plate reader. This resulted in the ladder of ADCC activity depicted numerically in FIG. 29 and graphically in FIG. 30. As demonstrated by the values in FIG. 29, this Fc engineering was successful in improving both the production yields and the stability as measured by the melting temperature (Tm). As a general trend, the more similar the Fc region is to the wild-type Fc 239 and 332 positions, the better the stability, but the lower the affinity for CD16 and the lower the ADCC and target cell killing activity. Test articles with only the S239D mutations, such as XENP41023, stood out from this trend in that they showed a significantly better improvement in stability compared to V90 than the constructs with only the 1332E variants, such as XENP41024, while also maintaining a similar (and even slightly stronger) affinity for CD16 compared with XENP41024. Overall, the results showed that S239D had less of a negative impact on stability and production than 1332E. The range of properties and affinities conferred by the different symmetric and asymmetric Fc variants disclosed may each provide unique advantages in certain contexts.

The monospecific and bispecific antibodies of the invention may utilize any of the heterodimeric Fc backbones depicted in FIGS. 12-16.

### F. Example 6: Characterizing NKp46×MICA/B bsAbs

To enhance the anti-tumor activities of MICA/B antibodies, multi-specific NK cell engaging antibodies were designed with the aim to simultaneously target MICA/B antigens and an orthogonal activating receptor NKp46. It was hypothesized that such bsAbs would have mechanisms of actions of ADCC, NKG2D agonism, blockade MICA and MICB cleavage, and NKp46 agonism (as depicted in FIG. 58).

### 6A. NKp46×MICA/B bsAb Synergistically Targets Multiple Activating Pathways

NK cells were co-cultured with A375-B2M-KO-RFP tumor cell line and NKp46×MICA/B or RSV×MICA/B bsAbs with WT effector function or KO effector function (i.e. ablated with PVA_/S267K to knockout CD16 binding) with or without NKG2D antibody that blocks MICA binding to the receptor. Tumor cell growth was assessed with Incucyte. The data as depicted in FIGS. 59 and 60 show that the NKp46×MICA/B bsAb synergistically targets multiple activating pathways resulting in superior IFNγ secretion and tumor cell kill.

### 6B. NKp46×MICA/B bsAbs are Superior to MICA/B mAbs

NK cells were co-cultured with A375-B2M-KO-RFP tumor cell lines and NKp46×MICA/B bsAbs in various formats and WT or KO effector function. Tumor growth and surface MICA density were assessed with Incucyte, data for which are depicted in FIGS. 61, 62, and 64. The data show that the bsAbs enabled more potent tumor cell kill than MICA/B, but only when crosslinking CD16.

### 6C. NKp46×MICA/B bsAbs Combine Synergistically with IL-15

NK cells were co-cultured with A375-B2M-KO-RFP tumor cell lines and NKp46×MICA/B bsAb or MICA/B mAb, either alone or in combination with IL-15-Fc fusion (sequences for an illustrative IL-15-Fc fusion is depicted in FIG. 66), and IFNγ secretion assessed. The data as depicted in FIG. 65 show that there was superior synergy in combining the NKp46×MICA/B bsAb with IL-15-Fc (in comparison to MICA/B mAb).

1. A MICA/B antigen binding domain comprising a set of vhCDR1-3 and vlCDR1-3 from a variable heavy domain and variable light domain pair selected from the group consisting of: (i) SEQ ID NOs: 240-2_42 for vhCDR1-3 and SEQ ID NOs: 244-246 for vlCDR1-3 of D94837_E11_1 [MICA/B]_H0_D94837_1E11_1 [MICA/B]_L0, (ii) SEQ ID NOs: 248-250 for vhCDR1-3 and SEQ ID NOs: 252-254 for vlCDR1-3 of D94837_E11_1 [MICA/B]_H1_D94837_1E11_1 [MICA/B]_L1, (iii) SEQ ID NOs: 256-258 for vhCDR1-3 and SEQ ID NOs: 260-262 for vlCDR1-3 of D94837_E11_1 [MICA/B]_H1_D94837_1E11_1 [MICA/B]_L2, (iv) SEQ ID NOs: 264-266 for vhCDR1-3 and SEQ ID NOs: 268-270 for vlCDR1-3 of D94837_E11_1 [MICA/B]_H2_D94837_1E11_1 [MICA/B]_L1, (v) SEQ ID NOs: 272-274 for vhCDR1-3 and SEQ ID NOs: 276-278 for vlCDR1-3 of D94837_E11_1 [MICA/B]_H2_D94837_1E11_1 [MICA/B]_L2, (vi) SEQ ID NOs: 280-282 for vhCDR1-3 and SEQ ID NOs: 284-286 for vlCDR1-3 of 2E5 [MICA/B]_H0_2E5 [MICA/B]_L0, (vii) SEQ ID NOs: 288-290 for vhCDR1-3 and SEQ ID NOs: 292-294 for vlCDR1-3 of 2E5 [MICA/B]_H1_2E5 [MICA/B]_L1, (viii) SEQ ID NOs: 296-298 for vhCDR1-3 and SEQ ID NOs: 300-302 for vlCDR1-3 of 2E5 [MICA/B]_H1_2E5 [MICA/B]_L2, (ix) SEQ ID NOs: 304-306 for vhCDR1-3 and SEQ ID NOs: 308-310 for vlCDR1-3 of 2E5 [MICA/B]_H2_2E5 [MICA/B]_L1, (x) SEQ ID NOs: 312-314 for vhCDR1-3 and SEQ ID NOs: 316-318 for vlCDR1-3 of 2E5 [MICA/B]_H2_2E5 [MICA/B]_L2, (xi) SEQ ID NOs: 320-322 for vhCDR1-3 and SEQ ID NOs: 324-326 for vlCDR1-3 of D94852_2E12 [MICA/B]_H0_D94852_2E12 [MICA/B]_L0, (xii) SEQ ID NOs: 328-330 for vhCDR1-3 and SEQ ID NOs: 332-334 for vlCDR1-3 of D94852_2E12 [MICA/B]_H1_D94852_2E12 [MICA/B]_L1, (xiii) SEQ ID NOs: 336-338 for vhCDR1-3 and SEQ ID NOs: 340-342 for v1CDR1-3 of D94852_2E12 [MICA/B]_H1_D94852_2E12 [MICA/B]_L2, (xiv) SEQ ID NOs: 344-346 for vhCDR1-3 and SEQ ID NOs: 348-350 for v1CDR1-3 of D94852_2E12 [MICA/B]_H2_D94852_2E12 [MICA/B]_L1, (xv) SEQ ID NOs: 352-354 for vhCDR1-3 and SEQ ID NOs: 356-358 for v1CDR1-3 of D94852_2E12 [MICA/B]_H2_D94852_2E12 [MICA/B]_L2, (xvi) SEQ ID NOs: 360-362 for vhCDR1-3 and SEQ ID NOs: 364-366 for v1CDR1-3 of D99136_2F7 [MICA/B]_H0_D99136_2F7 [MICA/B]_L0, (xvii) SEQ ID NOs: 368-370 for vhCDR1-3 and SEQ ID NOs: 372-374 for v1CDR1-3 of D99136_2F7 [MICA/B]_H1_D99136_2F7 [MICA/B]_L1, (xviii) SEQ ID NOs: 376-378 for vhCDR1-3 and SEQ ID NOs: 380-382 for v1CDR1-3 of D99136_2F7 [MICA/B]_H1_D99136_2F7 [MICA/B]_L2, (xix) SEQ ID NOs: 384-386 for vhCDR1-3 and SEQ ID NOs: 388-390 for v1CDR1-3 of D99136_2F7 [MICA/B]_H2_D99136_2F7 [MICA/B]_L1, (xx) SEQ ID NOs: 392-394 for vhCDR1-3 and SEQ ID NOs: 396-398 for v1CDR1-3 of D99136_2F7 [MICA/B]_H2_D99136_2F7 [MICA/B]_L2, (xxi) SEQ ID NOs: 400-402 for vhCDR1-3 and SEQ ID NOs: 404-406 for v1CDR1-3 of D103388_1C7 [MICA/B]_H0_D103388_1C7 [MICA/B]_L0, (xxii) SEQ ID NOs: 408-410 for vhCDR1-3 and SEQ ID NOs: 412-414 for v1CDR1-3 of D103388_1C7 [MICA/B]_H1_D103388_1C7 [MICA/B]_L1, (xxiii) SEQ ID NOs: 416-418 for vhCDR1-3 and SEQ ID NOs: 420-422 for v1CDR1-3 of D103388_1C7 [MICA/B]_H1_D103388_1C7 [MICA/B]_L2, (xxiv) SEQ ID NOs: 424-426 for vhCDR1-3 and SEQ ID NOs: 428-430 for v1CDR1-3 of D103388_1C7 [MICA/B]_H2_D103388_1C7 [MICA/B]_L1, (xxv) SEQ ID NOs: 432-434 for vhCDR1-3 and SEQ ID NOs: 436-438 for v1CDR1-3 of D103388_1C7 [MICA/B]_H2_D103388_1C7 [MICA/B]_L2, (xxvi) SEQ ID NOs: 440-442 for vhCDR1-3 and SEQ ID NOs: 444-446 for v1CDR1-3 of D103388_1D7 [MICA/B]_H0_D103388_1D7 [MICA/B]_L0, (xxvii) SEQ ID NOs: 448-450 for vhCDR1-3 and SEQ ID NOs: 452-454 for v1CDR1-3 of D103388_1D7 [MICA/B]_H1_D103388_1D7 [MICA/B]_L1, (xxviii) SEQ ID NOs: 456-458 for vhCDR1-3 and SEQ ID NOs: 460-462 for v1CDR1-3 of D103388_1D7 [MICA/B]_H1_D103388_1D7 [MICA/B]_L2, (xxix) SEQ ID NOs: 464-466 for vhCDR1-3 and SEQ ID NOs: 468-470 for vlCDR1-3 of D103388_1D7 [MICA/B]_H2_D103388_1D7 [MICA/B]_L1, (xxx) SEQ ID NOs: 472-474 for vhCDR1-3 and SEQ ID NOs: 476-478 for v1CDR1-3 of D103388_1D7 [MICA/B]_H2_D103388_1D7 [MICA/B]_L2, (xxxi) SEQ ID NOs: 480-482 for vhCDR1-3 and SEQ ID NOs: 484-486 for v1CDR1-3 of D105317_1A2 [MICA/B]_H0_D105317_1A2 [MICA/B]_L0, (xxxii) SEQ ID NOs: 488-490 for vhCDR1-3 and SEQ ID NOs: 492-494 for v1CDR1-3 of D105317_1A2 [MICA/B]_H1_D105317_1A2 [MICA/B]_L1, (xxxiii) SEQ ID NOs: 496-498 for vhCDR1-3 and SEQ ID NOs: 500-502 for v1CDR1-3 of D105317_1A2 [MICA/B]_H1_D105317_1A2 [MICA/B]_L2, (xxxiv) SEQ ID NOs: 504-506 for vhCDR1-3 and SEQ ID NOs: 508-510 for v1CDR1-3 of D105317_1A2 [MICA/B]_H2_D105317_1A2 [MICA/B]_L1, (xxxv) SEQ ID NOs: 512-514 for vhCDR1-3 and SEQ ID NOs: 516-518 for v1CDR1-3 of D105317_1A2 [MICA/B]_H2_D105317_1A2 [MICA/B]_L2, (xxxvi) SEQ ID NOs: 520-522 for vhCDR1-3 and SEQ ID NOs: 524-526 for v1CDR1-3 of D99136_2C11 [MICA/B]_H0_D99136_2C11 [MICA/B]_L0, (xxxvii) SEQ ID NOs: 528-530 for vhCDR1-3 and SEQ ID NOs: 532-534 for v1CDR1-3 of D99136_2C11 [MICA/B]_H1_D99136_2C11 [MICA/B]_L1, (xxxviii) SEQ ID NOs: 536-538 for vhCDR1-3 and SEQ ID NOs: 540-542 for v1CDR1-3 of D99136_2C11 [MICA/B]_H1_D99136_2C11 [MICA/B]_L2, (xxxix) SEQ ID NOs: 544-546 for vhCDR1-3 and SEQ ID NOs: 548-550 for v1CDR1-3 of D99136_2C11 [MICA/B]_H2_D99136_2C11 [MICA/B]_L1, (xl) SEQ ID NOs: 552-554 for vhCDR1-3 and SEQ ID NOs: 556-558 for v1CDR1-3 of D99136_2C11 [MICA/B]_H2_D99136_2C11 [MICA/B]_L2, (xli) SEQ ID NOs: 560-562 for vhCDR1-3 and SEQ ID NOs: 564-566 for v1CDR1-3 of D103388_1B11-2 [MICA/B]_H0_D103388_1B11-2 [MICA/B]_L0, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, and (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, as depicted in FIG. 21.

2. A MICA/B antigen binding domain comprising a variable heavy domain and variable light domain pair selected from the group consisting of: (i) SEQ ID Nos: 239 and 243 for D94837_1E11_1 [MICA/B]_H0_D94837_1E11_1 [MICA/B]_L0, (ii) SEQ ID Nos: 247and 251 for D94837_1E11_1 [MICA/B]_H1_D94837_1E11_1 [MICA/B]_L1, (iii) SEQ ID Nos: 255 and 259 for D94837_1E11_1 [MICA/B]_H1_D94837_1E11_1 [MICA/B]_L2, (iv) SEQ ID Nos: 263and 267 for D94837_1E11_1 [MICA/B]_H2_D94837_1E11_1 [MICA/B]_L1, (v) SEQ ID Nos: 271and 275 for D94837_1E11_1 [MICA/B]_H2_D94837_E11_1 [MICA/B]_L2, (vi) SEQ ID Nos: 279 and 283 for 2E5 [MICA/B]_H0_2E5 [MICA/B]_L0, (vii) SEQ ID Nos: 287and 291 for 2E5 [MICA/B]_H1_2E5 [MICA/B]_L1, (viii) SEQ ID Nos: 295and 299 for 2E5 [MICA/B]_H1_2E5 [MICA/B]_L2, (ix) SEQ ID Nos: 303 and 307 for 2E5 [MICA/B]_H2_2E5 [MICA/B]_L1, (x) SEQ ID Nos: 311and 315 for 2E5 [MICA/B]_H2_2E5 [MICA/B]_L2, (xi) SEQ ID Nos: 319 and 323 for D94852_2E12 [MICA/B]_H0_D94852_2E12 [MICA/B]_L0, (xii) SEQ ID Nos: 327 and 331 for D94852_2E12 [MICA/B]_H1_D94852_2E12 [MICA/B]_L1, (xiii) SEQ ID Nos: 335and 339 for D94852_2E12 [MICA/B]_H1_D94852_2E12 [MICA/B]_L2, (xiv) SEQ ID Nos: 343and 347 for D94852_2E12 [MICA/B]_H2_D94852_2E12 [MICA/B]_L1, (xv) SEQ ID Nos: 351 and 355 for D94852_2E12 [MICA/B]_H2_D94852_2E12 [MICA/B]_L2, (xvi) SEQ ID Nos: 359and 363 for D99136_2F7 [MICA/B]_H0_D99136_2F7 [MICA/B]_L0, (xvii) SEQ ID Nos: 367and 371 for D99136_2F7 [MICA/B]_H1_D99136_2F7 [MICA/B]_L1, (xviii) SEQ ID Nos: 375and 379 for D99136_2F7 [MICA/B]_H1_D99136_2F7 [MICA/B]_L2, (xix) SEQ ID Nos: 383 and 387 for D99136_2F7 [MICA/B]_H2_D99136_2F7 [MICA/B]_L1, (xx) SEQ ID Nos: 391 and 395 for D99136_2F7 [MICA/B]_H2_D99136_2F7 [MICA/B]_L2, (xxi) SEQ ID Nos: 399 and 403 for D103388_1C7 [MICA/B]_H0_D103388_1C7 [MICA/B]_L0, (xxii) SEQ ID Nos: 407 and 411 for D103388_1C7 [MICA/B]_H1_D103388_1C7 [MICA/B]_L1, (xxiii) SEQ ID Nos: 415 and 419 for D103388_1C7 [MICA/B]_H1_D103388_1C7 [MICA/B]_L2, (xxiv) SEQ ID Nos: 423 and 427 for D103388_1C7 [MICA/B]_H2_D103388_1C7 [MICA/B]_L1, (xxv) SEQ ID Nos: 431 and 435 for D103388_1C7 [MICA/B]_H2_D103388_1C7 [MICA/B]_L2, (xxvi) SEQ ID Nos: 439 and 443 for D103388_1D7 [MICA/B]_H0_D103388_1D7 [MICA/B]_L0, (xxvii) SEQ ID Nos: 447 and 451 for D103388_1D7 [MICA/B]_H1_D103388_1D7 [MICA/B]_L1, (xxviii) SEQ ID Nos: 455 and 459 for D103388_1D7 [MICA/B]_H1_D103388_1D7 [MICA/B]_L2, (xxix) SEQ ID Nos: 463 and 467 for D103388_1D7 [MICA/B]_H2_D103388_1D7 [MICA/B]_L1, (xxx) SEQ ID Nos: 471 and 475 for D103388_1D7 [MICA/B]_H2_D103388_1D7 [MICA/B]_L2, (xxxi) SEQ ID Nos: 479 and 483 for D105317_1A2 [MICA/B]_H0_D105317_1A2 [MICA/B]_L0, (xxxii) SEQ ID Nos: 487 and 491 for D105317_1A2 [MICA/B]_H1_D105317_1A2 [MICA/B]_L1, (xxxiii) SEQ ID Nos: 495 and 499 for D105317_1A2 [MICA/B]_H1_D105317_1A2 [MICA/B]_L2, (xxxiv) SEQ ID Nos: 503 and 507 for D105317_1A2 [MICA/B]_H2_D105317_1A2 [MICA/B]_L1, (xxxv) SEQ ID Nos: 511 and 515 for D105317_1A2 [MICA/B]_H2_D105317_1A2 [MICA/B]_L2, (xxxvi) SEQ ID Nos: 519 and 523 for D99136_2C11 [MICA/B]_H0_D99136_2C11 [MICA/B]_L0, (xxxvii) SEQ ID Nos: 527 and 531 for D99136_2C11 [MICA/B]_H1_D99136_2C11 [MICA/B]_L1, (xxxviii) SEQ ID Nos: 535 and 539 for D99136_2C11 [MICA/B]_H1_D99136_2C11 [MICA/B]_L2, (xxxix) SEQ ID Nos: 543 and 547 for D99136_2C11 [MICA/B]_H2_D99136_2C11 [MICA/B]_L1, (xl) SEQ ID Nos: 551 and 555 for D99136_2C11 [MICA/B]_H2_D99136_2C11 [MICA/B]_L2, (xli) SEQ ID Nos: 559 and 563 for D103388_1B11-2 [MICA/B]_H0_D103388_1B11-2 [MICA/B]_L0, (xlii) SEQ ID Nos: 567 and 571 for D105317_1B6 [MICA/B]_H0_D105317_1B6 [MICA/B]_L0, (xliii) SEQ ID Nos: 575 and 579 for D105317_1C8 [MICA/B]_H0_D105317_1C8 [MICA/B]_L0, (xliv) SEQ ID Nos: 583 and 587 for D105317_1F6 [MICA/B]_H0_D105317_1F6 [MICA/B]_L0, (xlv) SEQ ID Nos: 591 and 595 for D105317_1F7 [MICA/B]_H0_D105317_1F7 [MICA/B]_L0, (xlvi) SEQ ID Nos: 599 and 603 for D94837_1D3 [MICA/B]_H0_D94837_1D3 [MICA/B]_L0, (xlvii) SEQ ID Nos: 607 and 611 for D94837_1D8 [MICA/B]_H0_D94837_1D8 [MICA/B]_L0, (xlviii) SEQ ID Nos: 615 and 619 for D94837_1D9 [MICA/B]_H0_D94837_1D9 [MICA/B]_L0, (xlix) SEQ ID Nos: 623 and 627 for D94837_1E1 [MICA/B]_H0_D94837_1E1 [MICA/B]_L0, (1) SEQ ID Nos: 631 and 635 for D94852_2D4 [MICA/B]_H0_D94852_2D4 [MICA/B]_L0, (li) SEQ ID Nos: 639 and 643 for D94852_2G8 [MICA/B]_H0_D94852_2G8 [MICA/B]_L0, (lii) SEQ ID Nos: 647 and 651 for D88487_2A8 [MICA/B]_H0_D88487_2A8 [MICA/B]_L0, (liii) SEQ ID Nos: 655 and 659 for D94837_3B12 [MICA/B]_H0_D94837_3B12 [MICA/B]_L0, (liv) SEQ ID Nos: 663 and 667 for D99122_1H7 [MICA/B]_H0_D99122_1H7 [MICA/B]_L0, (lv) SEQ ID Nos: 671 and 675 for D99136_2E8 [MICA/B]_H0_D99136_2E8 [MICA/B]_L0, and (lvi) SEQ ID Nos: 647 and 651 for D88487_2A8 [MICA/B]_H0_D88487_2A8 [MICA/B]_L0, as depicted in FIG. 21.

3. An antibody comprising a MICA/B antigen binding domain according to claim 1.

4. The antibody according to claim 3, wherein the antibody is a monoclonal antibody.

5. The antibody according to claim 3, wherein the antibody is a bispecific antibody.

6. A composition comprising a MICA/B antigen binding domain according to claim 1.

7. A nucleic acid composition comprising (a) a first nucleic acid encoding the variable heavy domain according to claim 1; and (b) a second nucleic acid encoding the variable light domain according to claim 1.

8. An expression vector composition comprising (a) a first expression vector comprising the first nucleic acid according to claim 7; and (b) a second expression vector comprising the second nucleic acid composition according to claim 7.

9. A host cell comprising the expression vector composition according to claim 8.

10. A method of making a MICA/B antigen binding domain or an antibody comprising such, the method comprising culturing a host cell according to claim 9 under conditions wherein the MICA/B antigen binding domain or the antibody comprising such is expressed, and recovering the MICA/B antigen binding domain or the antibody comprising such.

11. A NKp46 antigen binding domain comprising a set of vhCDR1-3 and vlCDR1-3 from a variable heavy domain and variable light domain pair selected from the group consisting of: SEQ ID NOs: 20-222 for vhCDR1-3 and SEQ ID NOs: 724-726 for vlCDR1-3 of 2C10A3.372[NKp46]_H1_2C10A3.372[NKp46]_L1, as depicted in FIG. 23.

12. A NKp46 antigen binding domain comprising a variable heavy domain and variable light domain pair selected from the group consisting of SEQ ID Nos: 719 and 723 for 2C10A3.372[NKp46]_H1_2C10A3.372[NKp46]_L1, as depicted in FIG. 23.

13.-20. (canceled)

21. A heterodimeric antibody, comprising:
(a) a first monomer, comprising:
(i) an anti-NKp46 scFv comprising a first variable heavy VH1 domain, an scFv linker, and a first variable light VL1 domain; and
(ii) a first Fc domain, wherein the scFv is covalently attached to the N-terminus of the first Fc domain using a domain linker;
(b) a second monomer comprising a VH2-CH1-hinge-CH2-CH3 monomer, wherein VH2 is a second variable heavy domain and CH2-CH3 is a second Fc domain; and
(c) a light chain comprising a second variable light VL2 domain, wherein the second variable heavy VH2 domain and the second variable light VL2 domain form a MICA/B antigen binding domain.

22. The heterodimeric antibody according to claim 21, wherein the MICA/B antigen binding domain comprises a set of vhCDR1-3 and vlCDR1-3 from a variable heavy domain and variable light domain pair selected from the group consisting of: (i) SEQ ID NOs: 240-242 for vhCDR1-3 and SEQ ID NOs: 244-246 for vlCDR1-3 of D94837_1E11_1 [MICA/B]_H0_D94837_1E11_1 [MICA/B]_L0, (ii) SEQ ID NOs: 248-250 for vhCDR1-3 and SEQ ID NOs: 252-254 for vlCDR1-3 of D94837_1E11_1 [MICA/B]_H1_D94837_1E11_1 [MICA/B]_L1, (iii) SEQ ID NOs: 256-258 for vhCDR1-3 and SEQ ID NOs: 260-262 for vlCDR1-3 of D94837_1E11_1 [MICA/B]_H1_D94837_1E11_1 [MICA/B]_L2, (iv) SEQ ID NOs: 264-266 for vhCDR1-3 and SEQ ID NOs: 268-270 for vlCDR1-3 of D94837_1E11_1 [MICA/B]_H2_D94837_1E11_1 [MICA/B]_L1, (v) SEQ ID NOs: 272-274 for vhCDR1-3 and SEQ ID NOs: 276-278 for v1CDR1-3 of D94837_E11_1 [MICA/B]_H2_D94837_1E11_1 [MICA/B]_L2, (vi) SEQ ID NOs: 280-282 for vhCDR1-3 and SEQ ID NOs: 284-286 for v1CDR1-3 of 2E5 [MICA/B]_H0_2E5 [MICA/B]_L0, (vii) SEQ ID NOs: 288-290 for vhCDR1-3 and SEQ ID NOs: 292-294 for v1CDR1-3 of 2E5 [MICA/B]_H1_2E5 [MICA/B]_L1, (viii) SEQ ID NOs: 296-298 for vhCDR1-3 and SEQ ID NOs: 300-302 for v1CDR1-3 of 2E5 [MICA/B]_H1_2E5 [MICA/B]_L2, (ix) SEQ ID NOs: 304-306 for vhCDR1-3 and SEQ ID NOs: 308-310 for v1CDR1-3 of 2E5 [MICA/B]_H2_2E5 [MICA/B]_L1, (x) SEQ ID NOs: 312-314 for vhCDR1-3 and SEQ ID NOs: 316-318 for v1CDR1-3 of 2E5 [MICA/B]_H2_2E5 [MICA/B]_L2, (xi) SEQ ID NOs: 320-322 for vhCDR1-3 and SEQ ID NOs: 324-326 for vlCDR1-3 of D94852_2E12 [MICA/B]_H0_D94852_2E12 [MICA/B]_L0, (xii) SEQ ID NOs: 328-330 for vhCDR1-3 and SEQ ID NOs: 332-334 for vlCDR1-3 of D94852_2E12 [MICA/B]_H1_D94852_2E12 [MICA/B]_L1, (xiii) SEQ ID NOs: 336-338 for vhCDR1-3 and SEQ ID NOs: 340-342 for vlCDR1-3 of D94852_2E12 [MICA/B]_H1_D94852_2E12 [MICA/B]_L2, (xiv) SEQ ID NOs: 344-346 for vhCDR1-3 and SEQ ID NOs: 348-350 for vlCDR1-3 of D94852_2E12 [MICA/B]_H2_D94852_2E12 [MICA/B]_L1, (xv) SEQ ID NOs: 352-354 for vhCDR1-3 and SEQ ID NOs: 356-358 for vlCDR1-3 of D94852_2E12 [MICA/B]_H2_D94852_2E12 [MICA/B]_L2, (xvi) SEQ ID NOs: 360-362 for vhCDR1-3 and SEQ ID NOs: 364-366 for vlCDR1-3 of D99136_2F7 [MICA/B]_H0_D99136_2F7 [MICA/B]_L0, (xvii) SEQ ID NOs: 368-370 for vhCDR1-3 and SEQ ID NOs: 372-374 for vlCDR1-3 of D99136_2F7 [MICA/B]_H1_D99136_2F7 [MICA/B]_L1, (xviii) SEQ ID NOs: 376-378 for vhCDR1-3 and SEQ ID NOs: 380-382 for vlCDR1-3 of D99136_2F7 [MICA/B]_H1_D99136_2F7 [MICA/B]_L2, (xix) SEQ ID NOs: 384-386 for vhCDR1-3 and SEQ ID NOs: 388-390 for vlCDR1-3 of D99136_2F7 [MICA/B]_H2_D99136_2F7 [MICA/B]_L1, (xx) SEQ ID NOs: 392-394 for vhCDR1-3 and SEQ ID NOs: 396-398 for vlCDR1-3 of D99136_2F7 [MICA/B]_H2_D99136_2F7 [MICA/B]_L2, (xxi) SEQ ID NOs: 400-402 for vhCDR1-3 and SEQ ID NOs: 404-406 for vlCDR1-3 of D103388_1C7 [MICA/B]_H0_D103388_1C7 [MICA/B]_L0, (xxii) SEQ ID NOs: 408-410 for vhCDR1-3 and SEQ ID NOs: 412-414 for vlCDR1-3 of D103388_1C7 [MICA/B]_H1_D103388_1C7 [MICA/B]_L1, (xxiii) SEQ ID NOs: 416-418 for vhCDR1-3 and SEQ ID NOs: 420-422 for vlCDR1-3 of D103388_1C7 [MICA/B]_H1_D103388_1C7 [MICA/B]_L2, (xxiv) SEQ ID NOs: 424-426 for vhCDR1-3 and SEQ ID NOs: 428-430 for vlCDR1-3 of D103388_1C7 [MICA/B]_H2_D103388_1C7 [MICA/B]_L1, (xxv) SEQ ID NOs: 432-434 for vhCDR1-3 and SEQ ID NOs: 436-438 for vlCDR1-3 of D103388_1C7 [MICA/B]_H2_D103388_1C7 [MICA/B]_L2, (xxvi) SEQ ID NOs: 440-442 for vhCDR1-3 and SEQ ID NOs: 444-446 for vlCDR1-3 of D103388_1D7 [MICA/B]_H0_D103388_1D7 [MICA/B]_L0, (xxvii) SEQ ID NOs: 448-450 for vhCDR1-3 and SEQ ID NOs: 452-454 for v1CDR1-3 of D103388_1D7 [MICA/B]_H1_D103388_1D7 [MICA/B]_L1, (xxviii) SEQ ID NOs: 456-458 for vhCDR1-3 and SEQ ID NOs: 460-462 for v1CDR1-3 of D103388_1D7 [MICA/B]_H1_D103388_1D7 [MICA/B]_L2, (xxix) SEQ ID NOs: 464-466 for vhCDR1-3 and SEQ ID NOs: 468-470 for v1CDR1-3 of D103388_1D7 [MICA/B]_H2_D103388_1D7 [MICA/B]_L1, (xxx) SEQ ID NOs: 472-474 for vhCDR1-3 and SEQ ID NOs: 476-478 for v1CDR1-3 of D103388_1D7 [MICA/B]_H2_D103388_1D7 [MICA/B]_L2, (xxxi) SEQ ID NOs: 480-482 for vhCDR1-3 and SEQ ID NOs: 484-486 for v1CDR1-3 of D105317_1A2 [MICA/B]_H0_D105317_1A2 [MICA/B]_L0, (xxxii) SEQ ID NOs: 488-490 for vhCDR1-3 and SEQ ID NOs: 492-494 for v1CDR1-3 of D105317_1A2 [MICA/B]_H1_D105317_1A2 [MICA/B]_L1, (xxxiii) SEQ ID NOs: 496-498 for vhCDR1-3 and SEQ ID NOs: 500-502 for v1CDR1-3 of D105317_1A2 [MICA/B]_H1_D105317_1A2 [MICA/B]_L2, (xxxiv) SEQ ID NOs: 504-506 for vhCDR1-3 and SEQ ID NOs: 508-510 for v1CDR1-3 of D105317_1A2 [MICA/B]_H2_D105317_1A2 [MICA/B]_L1, (xxxv) SEQ ID NOs: 512-514 for vhCDR1-3 and SEQ ID NOs: 516-518 for v1CDR1-3 of D105317_1A2 [MICA/B]_H2_D105317_1A2 [MICA/B]_L2, (xxxvi) SEQ ID NOs: 520-522 for vhCDR1-3 and SEQ ID NOs: 524-526 for v1CDR1-3 of D99136_2C11 [MICA/B]_H0_D99136_2C11 [MICA/B]_L0, (xxxvii) SEQ ID NOs: 528-530 for vhCDR1-3 and SEQ ID NOs: 532-534 for v1CDR1-3 of D99136_2C11 [MICA/B]_H1_D99136_2C11 [MICA/B]_L1, (xxxviii) SEQ ID NOs: 536-538 for vhCDR1-3 and SEQ ID NOs: 540-542 for v1CDR1-3 of D99136_2C11 [MICA/B]_H1_D99136_2C11 [MICA/B]_L2, (xxxix) SEQ ID NOs: 544-546 for vhCDR1-3 and SEQ ID NOs: 548-550 for v1CDR1-3 of D99136_2C11 [MICA/B]_H2_D99136_2C11 [MICA/B]_L1, (xl) SEQ ID NOs: 552-554 for vhCDR1-3 and SEQ ID NOs: 556-558 for v1CDR1-3 of D99136_2C11 [MICA/B]_H2_D99136_2C11 [MICA/B]_L2, (xli) SEQ ID NOs: 560-562 for vhCDR1-3 and SEQ ID NOs: 564-566 for v1CDR1-3 of D103388_1B11-2 [MICA/B]_H0_D103388_1B11-2 [MICA/B]_L0, (xlii) SEQ ID NOs: 568-570 for vhCDR1-3 and SEQ ID NOs: 572-574 for vlCDR1-3 of D105317_1B6 [MICA/B]_H0_D105317_1B6 [MICA/B]_L0, (xliii) SEQ ID NOs: 576-578 for vhCDR1-3 and SEQ ID NOs: 580-582 for vlCDR1-3 of D105317_1C8 [MICA/B]_H0_D105317_1C8 [MICA/B]_L0, (xliv) SEQ ID NOs: 584-586 for vhCDR1-3 and SEQ ID NOs: 588-590 for vlCDR1-3 of D105317_1F6 [MICA/B]_H0_D105317_1F6 [MICA/B]_L0, (xlv) SEQ ID NOs: 592-594 for vhCDR1-3 and SEQ ID NOs: 596-598 for vlCDR1-3 of D105317_1F7 [MICA/B]_H0_D105317_1F7 [MICA/B]_L0, (xlvi) SEQ ID NOs: 600-602 for vhCDR1-3 and SEQ ID NOs: 604-606 for vlCDR1-3 of D94837_1D3 [MICA/B]_H0_D94837_1D3 [MICA/B]_L0, (xlvii) SEQ ID NOs: 608-610 for vhCDR1-3 and SEQ ID NOs: 612-614 for vlCDR1-3 of D94837_1D8 [MICA/B]_H0_D94837_1D8 [MICA/B]_L0, (xlviii) SEQ ID NOs: 616-618 for vhCDR1-3 and SEQ ID NOs: 620-622 for vlCDR1-3 of D94837_1D9 [MICA/B]_H0_D94837_1D9 [MICA/B]_L0, (xlix) SEQ ID NOs: 624-626 for vhCDR1-3 and SEQ ID NOs: 628-630 for vlCDR1-3 of D94837_1E1 [MICA/B]_H0_D94837_1E1 [MICA/B]_L0, (1) SEQ ID NOs: 632-634 for vhCDR1-3 and SEQ ID NOs: 636-638 for vlCDR1-3 of D94852_2D4 [MICA/B]_H0_D94852_2D4 [MICA/B]_L0, (li) SEQ ID NOs: 640-642 for vhCDR1-3 and SEQ ID NOs: 644-646 for vlCDR1-3 of D94852_2G8 [MICA/B]_H0_D94852_2G8 [MICA/B]_L0, (lii) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (liii) SEQ ID NOs: 656-658 for vhCDR1-3 and SEQ ID NOs: 660-662 for vlCDR1-3 of D94837_3B12 [MICA/B]_H0_D94837_3B12 [MICA/B]_L0, (liv) SEQ ID NOs: 664-666 for vhCDR1-3 and SEQ ID NOs: 668-670 for vlCDR1-3 of D99122_1H7 [MICA/B]_H0_D99122_1H7 [MICA/B]_L0, (lv) SEQ ID NOs: 672-674 for vhCDR1-3 and SEQ ID NOs: 676-678 for vlCDR1-3 of D99136_2E8 [MICA/B]_H0_D99136_2E8 [MICA/B]_L0, (lvi) SEQ ID NOs: 648-650 for vhCDR1-3 and SEQ ID NOs: 652-654 for vlCDR1-3 of D88487_2A8 [MICA/B]_H0_D88487_2A8 [MICA/B]_L0, (lvii) SEQ ID NOs: 680-682 for vhCDR1-3 and SEQ ID NOs: 684-686 for vlCDR1-3 of 3F9[MICA/B]_H0_3F9[MICA/B]_L0, (lviii) SEQ ID NOs: 688-690 for vhCDR1-3 and SEQ ID NOs: 692-694 for vlCDR1-3 of 6E1[MICA/B]_H0_6E1[MICA/B]_L0, (lix) SEQ ID NOs: 696-698 for vhCDR1-3 and SEQ ID NOs: 700-702 for vlCDR1-3 of 7C6[MICA/B]_H0_7C6[MICA/B]_L0, (lx) SEQ ID NOs: 704-706 for vhCDR1-3 and SEQ ID NOs: 708-710 for vlCDR1-3 of 13A9 [MICA/B]_H0_13A9 [MICA/B]_L0, and (lxi) SEQ ID NOs: 712-714 for vhCDR1-3 and SEQ ID NOs: 716-718 for vlCDR1-3 of 1D5 [MICA/B]_H0_1D5 [MICA/B]_L0, as depicted in FIGS. 21 and 22.

23. The heterodimeric antibody according to claim 21, wherein the MICA/B antigen binding domain comprises a variable heavy domain and variable light domain pair selected from the group consisting of: (i) SEQ ID Nos: 239 and 243 for D94837_1E11_1 [MICA/B]_H0_D94837_1E1_1 [MICA/B]_L0, (ii) SEQ ID Nos: 247and 251 for D94837_1E11_1 [MICA/B]_H1_D94837_1E11_1 [MICA/B]_L1, (iii) SEQ ID Nos: 255 and 259 for D94837_1E11_1 [MICA/B]_H1_D94837_1E11_1 [MICA/B]_L2, (iv) SEQ ID Nos: 263and 267 for D94837_1E11_1 [MICA/B]_H2_D94837_1E11_1 [MICA/B]_L1, (v) SEQ ID Nos: 271and 275 for D94837_1E11_1 [MICA/B]_H2_D94837_1E11_1 [MICA/B]_L2, (vi) SEQ ID Nos: 279 and 283 for 2E5 [MICA/B]_H0_2E5 [MICA/B]_L0, (vii) SEQ ID Nos: 287and 291 for 2E5 [MICA/B]_H1_2E5 [MICA/B]_L1, (viii) SEQ ID Nos: 295and 299 for 2E5 [MICA/B]_H1_2E5 [MICA/B]_L2, (ix) SEQ ID Nos: 303 and 307 for 2E5 [MICA/B]_H2_2E5 [MICA/B]_L1, (x) SEQ ID Nos: 311and 315 for 2E5 [MICA/B]_H2_2E5 [MICA/B]_L2, (xi) SEQ ID Nos: 319 and 323 for D94852_2E12 [MICA/B]_H0_D94852_2E12 [MICA/B]_L0, (xii) SEQ ID Nos: 327 and 331 for D94852_2E12 [MICA/B]_H1_D94852_2E12 [MICA/B]_L1, (xiii) SEQ ID Nos: 335[[XXX] and 339 for D94852_2E12 [MICA/B]_H1_D94852_2E12 [MICA/B]_L2, (xiv) SEQ ID Nos: 343and 347 for D94852_2E12 [MICA/B]_H2_D94852_2E12 [MICA/B]_L1, (xv) SEQ ID Nos: 351 and 355 for D94852_2E12 [MICA/B]_H2_D94852_2E12 [MICA/B]_L2, (xvi) SEQ ID Nos: 359and 363 for D99136_2F7 [MICA/B]_H0_D99136_2F7 [MICA/B]_L0, (xvii) SEQ ID Nos: 367[[XXX] and 371 for D99136_2F7 [MICA/B]_H1_D99136_2F7 [MICA/B]_L1, (xviii) SEQ ID Nos: 375and 379 for D99136_2F7 [MICA/B]_H1_D99136_2F7 [MICA/B]_L2, (xix) SEQ ID Nos: 383 and 387 for D99136_2F7 [MICA/B]_H2_D99136_2F7 [MICA/B]_L1, (xx) SEQ ID Nos: 391 and 395 for D99136_2F7 [MICA/B]_H2_D99136_2F7 [MICA/B]_L2, (xxi) SEQ ID Nos: 399 and 403 for D103388_1C7 [MICA/B]_H0_D103388_1C7 [MICA/B]_L0, (xxii) SEQ ID Nos: 407 and 411 for D103388_1C7 [MICA/B]_H1_D103388_1C7 [MICA/B]_L1, (xxiii) SEQ ID Nos: 415 and 419 for D103388_1C7 [MICA/B]_H1_D103388_1C7 [MICA/B]_L2, (xxiv) SEQ ID Nos: 423 and 427 for D103388_1C7 [MICA/B]_H2_D103388_1C7 [MICA/B]_L1, (xxv) SEQ ID Nos: 431 and 435 for D103388_1C7 [MICA/B]_H2_D103388_1C7 [MICA/B]_L2, (xxvi) SEQ ID Nos: 439 and 443 for D103388_1D7 [MICA/B]_H0_D103388_1D7 [MICA/B]_L0, (xxvii) SEQ ID Nos: 447 and 451 for D103388_1D7 [MICA/B]_H1_D103388_1D7 [MICA/B]_L1, (xxviii) SEQ ID Nos: 455 and 459 for D103388_1D7 [MICA/B]_H1_D103388_1D7 [MICA/B]_L2, (xxix) SEQ ID Nos: 463 and 467 for D103388_1D7 [MICA/B]_H2_D103388_1D7 [MICA/B]_L1, (xxx) SEQ ID Nos: 471 and 475 for D103388_1D7 [MICA/B]_H2_D103388_1D7 [MICA/B]_L2, (xxxi) SEQ ID Nos: 479 and 483 for D105317_1A2 [MICA/B]_H0_D105317_1A2 [MICA/B]_L0, (xxxii) SEQ ID Nos: 487 and 491 for D105317_1A2 [MICA/B]_H1_D105317_1A2 [MICA/B]_L1, (xxxiii) SEQ ID Nos: 495 and 499 for D105317_1A2 [MICA/B]_H1_D105317_1A2 [MICA/B]_L2, (xxxiv) SEQ ID Nos: 503 and 507 for D105317_1A2 [MICA/B]_H2_D105317_1A2 [MICA/B]_L1, (xxxv) SEQ ID Nos: 511 and 515 for D105317_1A2 [MICA/B]_H2_D105317_1A2 [MICA/B]_L2, (xxxvi) SEQ ID Nos: 519 and 523 for D99136_2C11 [MICA/B]_H0_D99136_2C11 [MICA/B]_L0, (xxxvii) SEQ ID Nos: 527 and 531 for D99136_2C11 [MICA/B]_H1_D99136_2C11 [MICA/B]_L1, (xxxviii) SEQ ID Nos: 535 and 539 for D99136_2C11 [MICA/B]_H1_D99136_2C11 [MICA/B]_L2, (xxxix) SEQ ID Nos: 543 and 547 for D99136_2C11 [MICA/B]_H2_D99136_2C11 [MICA/B]_L1, (xl) SEQ ID Nos: 551 and 555 for D99136_2C11 [MICA/B]_H2_D99136_2C11 [MICA/B]_L2, (xli) SEQ ID Nos: 559 and 563 for D103388_1B11-2 [MICA/B]_H0_D103388_1B11-2 [MICA/B]_L0, (xlii) SEQ ID Nos: 567 and 571 for D105317_1B6 [MICA/B]_H0_D105317_1B6 [MICA/B]_L0, (xliii) SEQ ID Nos: 575 and 579 for D105317_1C8 [MICA/B]_H0_D105317_1C8 [MICA/B]_L0, (xliv) SEQ ID Nos: 583 and 587 for D105317_1F6 [MICA/B]_H0_D105317_1F6 [MICA/B]_L0, (xlv) SEQ ID Nos: 591 and 595 for D105317_1F7 [MICA/B]_H0_D105317_1F7 [MICA/B]_L0, (xlvi) SEQ ID Nos: 599 and 603 for D94837_1D3 [MICA/B]_H0_D94837_1D3 [MICA/B]_L0, (xlvii) SEQ ID Nos: 607 and 611 for D94837_1D8 [MICA/B]_H0_D94837_1D8 [MICA/B]_L0, (xlviii) SEQ ID Nos: 615 and 619 for D94837_1D9 [MICA/B]_H0_D94837_1D9 [MICA/B]_L0, (xlix) SEQ ID Nos: 623 and 627 for D94837_1E1 [MICA/B]_H0_D94837_1E1 [MICA/B]_L0, (1) SEQ ID Nos: 631 and 635 for D94852_2D4 [MICA/B]_H0_D94852_2D4 [MICA/B]_L0, (li) SEQ ID Nos: 639 and 643 for D94852_2G8 [MICA/B]_H0_D94852_2G8 [MICA/B]_L0, (lii) SEQ ID Nos: 647 and 651 for D88487_2A8 [MICA/B]_H0_D88487_2A8 [MICA/B]_L0, (liii) SEQ ID Nos: 655 and 659 for D94837_3B12 [MICA/B]_H0_D94837_3B12 [MICA/B]_L0, (liv) SEQ ID Nos: 663 and 667 for D99122_1H7 [MICA/B]_H0_D99i22_1H7 [MICA/B]_L0, (lv) SEQ ID Nos: 671 and 675 for D99136_2E8 [MICA/B]_H0_D99136_2E8 [MICA/B]_L0, (lvi) SEQ ID Nos: 647 and 651 for D88487_2A8 [MICA/B]_H0_D88487_2A8 [MICA/B]_L0, (lvii) SEQ ID Nos: 679 and 683 for 3F9[MICA/B]_H0_3F9[MICA/B]_L0, (lviii) SEQ ID Nos: 687 and 691 for 6E1[MICA/B]_H0_6E1[MICA/B]_L0, (lix) SEQ ID Nos: 695 and 699 for 7C6[MICA/B]_H0_7C6[MICA/B]_L0, (lx) SEQ ID Nos: 703 and 707 for 13A9 [MICA/B]_H0_13A9 [MICA/B]_L0, and (lxi) SEQ ID Nos: 711 and 21 for 1D5 [MICA/B]_H0_1D5 [MICA/B]_L0, as depicted in FIGS. 21 and 22.

24. The heterodimeric antibody according to claim 21, wherein the anti-NKp46 scFv comprises a set of vhCDR1-3 and vlCDR1-3 from a first variable heavy VH1 domain and first variable light VL1 domain pair selected from the group consisting of: (i) SEQ ID NOs: 720-722 for vhCDR1-3 and SEQ ID NOs: 724-726 for vlCDR1-3 of 2C10A3.372[NKp46]_H1_2C10A3.372[NKp46]_L1, and (ii) SEQ ID NOs: 728-730 for vhCDR1-3 and SEQ ID NOs: 732-734 for vlCDR1-3 of NKp46-A[NKp46]_H_NKp46-A[NKp46]_L, as depicted in FIGS. 23 and 24.

25. The heterodimeric antibody according to claim 21, wherein the anti-NKp46 scFv comprises a variable heavy domain and variable light domain pair selected from the group consisting of: (i) SEQ ID Nos: 719 and 72 for 2C10A3.372[NKp46]_H1_2C10A3.372[NKp46]_L1, and (ii) SEQ ID Nos: 727 and 731 for NKp46-A[NKp46]_H_NKp46-A[NKp46]_L, as depicted in FIGS. 23 and 24.

26. The heterodimeric antibody according to claim 21, wherein the first variable light VL1 domain of the anti-NKp46 scFv is covalently attached to the N-terminus of the first Fc domain using a domain linker.

27. The heterodimeric antibody according to claim 21, wherein the first variable heavy VH1 domain of the anti-NKp46 scFv is covalently attached to the N-terminus of the first Fc domain using a domain linker.

28. The heterodimeric antibody according to claim 21, selected from the group consisting of: (i) the amino acid sequences of SEQ ID NOs: 794-796 of XENP46810, (ii) the amino acid sequences of SEQ ID NOS: 797-799 of XENP46811, and (iii) the amino acid sequences of SEQ ID NOs: 800-802 of XENP46812, as depicted in FIG. 31.

29.-35. (canceled)

36. The heterodimeric antibody according to claim 21, wherein the scFv linker is a charged scFv linker.

37. The heterodimeric antibody according to claim 36, wherein the scFv linker is a charged scFv linker having the amino acid sequence (GKPGS)₄ (SEQ ID NO: 1).

38. The heterodimeric antibody according to claim 21, wherein the first and second Fc domains are each variant Fc domains.

39. The heterodimeric antibody according to claim 38, wherein the first and/or second variant Fc domains comprise one or more FcγRIIIA (CD16a) binding variant substitutions.

40. The heterodimeric antibody according to claim 39, wherein the one or more FcγRIIIA (CD16a) binding variant substitutions are selected from the group consisting of: (i) 236A, (ii) 239D, (iii) 239E, (iv) 243L, (v) 298A, (vi) 299T, (vii) 332E, (viii) 332D, (ix) 239D/332E, (x) 236A/332E, (xi) 239D/332E/330L, and (xii) 332E/330L, wherein numbering is according to EU numbering.

41. The heterodimeric antibody according to claim 40, wherein the first and second variant Fc domains comprise a set of FcγRIIIA (CD16a) binding variant substitutions selected from the group consisting of: (i) S239D/I332E: S239D/I332E, (ii) S239D S239D, (iii) 1332E: 1332E, (iv) WT: S239D/I332E, (v) WT: S239D, (vi) WT: 1332E, (vii) S239D/I332E: WT, (viii) S239D: WT, (ix) 1332E: WT, (x) S239D/I332E: S239D, (xi) S239D/I332E: 1332E, (xii) S239D: S239D/I332E, (xiii) 1332E: S239D/I332E, (xiv) S239D: 1332E, and (xv) 1332E: S239D, wherein numbering is according to EU numbering.

42. The heterodimeric antibody according to claim 41, wherein the first and/or second variant Fc domains comprise the FcγRIIIA (CD16a) binding variant substitutions of S239D/I332E, wherein numbering is according to EU numbering.

43. The heterodimeric antibody according to claim 38, wherein the first and second variant Fc domains comprise a set of heterodimerization variants selected from the group consisting of those depicted in FIGS. 4A-4F, wherein numbering is according to EU numbering.

44. The heterodimeric antibody according to claim 43, wherein the set of heterodimerization variants is selected from the group consisting of: (i) S364K/E357Q: L368D/K370S, (ii) S364K: L368D/K370S, (iii) S364K: L368E/K370S, (iv) D401K: T411E/K360E/Q362E, and (v) T366W: T366S/L368A/Y407V, wherein numbering is according to EU numbering.

45. The heterodimeric antibody according to claim 38, wherein the first and second variant Fc domains further comprise one or more ablation variants.

46. The heterodimeric antibody according to claim 45, wherein the one or more ablation variants are E233P/L234V/L235A/G236del/S267K, wherein numbering is according to EU numbering.

47. The heterodimeric antibody according to claim 21, wherein one of the first or second variant Fc domain comprises one or more pI variants.

48. The heterodimeric antibody according to claim 47, wherein the one or more pI variants are N208D/Q295E/N384D/Q418E/N421D, wherein numbering is according to EU numbering.

49. The heterodimeric antibody according to claim 21, wherein the first monomer comprises amino acid variants S364K/E357Q/E233P/L234V/L235A/G236del/S267K, wherein the second monomer comprises amino acid variants L368D/K370S/N208D/Q295E/N384D/Q418E/N421D/E233P/L234V/L235A/G236del/S267K, and wherein numbering is according to EU numbering.

50. The heterodimeric antibody according to claim 21, wherein the first and second monomers each further comprise amino acid variants M428L/N434S, M428L/434A, or M252Y/S254T/T256E, wherein numbering is according to EU numbering.

51. A nucleic acid composition comprising nucleic acids encoding the first and second monomers and the light chain of the antibody according to claim 21.

52. An expression vector comprising the nucleic acids according to claim 51.

53. A host cell transformed with an expression vector according to claim 52.

54. A method of making a heterodimeric antibody comprising:
(a) culturing the host cell according to claim 53 under conditions wherein the heterodimeric antibody is expressed; and
(b) recovering the heterodimeric antibody.

55.-62. (canceled)

63. A heterodimeric antibody, comprising:
(a) a first monomer comprising, from N-terminus to C-terminus, a VH1-CH1-[first optional domain linker]-scFv-[second optional domain linker]-CH2-CH3, wherein VH1 is a first variable heavy VH1 domain, scFv is an anti-NKp46 scFv, and CH2-CH3 is a first Fc domain;
(b) a second monomer comprising, from N-terminus to C-terminus, a CH2-CH3, wherein the CH2-CH3 is a second Fc domain; and
(c) a light chain comprising, from N-terminus to C-terminus, a VL1-CL, wherein VL1 is a first variable light VH1 domain and CL is a constant light domain,
wherein: (i) the first variable heavy VH1 domain and the first variable light VL1 domain form a MICA/B antigen binding domain, (ii) the anti-NKp46 scFv comprises a second variable heavy VH2 domain, a scFv linker, and a second variable light VL2 domain, and
(iii) the anti-NKp46 scFv is covalently attached between the C-terminus of the CH1 domain and the N-terminus of the first Fc domain.

64.-88. (canceled)

89. A heterodimeric antibody, comprising:
(a) a first monomer comprising, from N-terminus to C-terminus, a VH1-CH1-first linker-scFv-second linker-CH2-CH3, wherein the VH1 is a first variable heavy domain, the scFv is an anti-NKp46 scFv, and the CH2-CH3 is a first Fc domain;
(b) a second monomer comprising, from N-terminus to C-terminus, a VH2-CH1-hinge-CH2-CH3, wherein the VH2 is a second variable heavy domain and the CH2-CH3 is a second Fc domain; and
(c) a common light chain comprising, from N-terminus to C-terminus, a VL-CL, wherein the VL is a variable light domain, and the CL is a light chain constant domain,
wherein the first variable heavy VH1 domain and the variable light VL domain form a first MICA/B antigen binding domain, and the second variably heavy VH2 domain and the variable light VL domain form a second MICA/B antigen binding domain.

90.-122. (canceled)

123. A heterodimeric antibody, comprising:
(a) a first monomer comprising, from N-terminus to C-terminus, a VH1-CH1-hinge-first linker-VH1-CH1-hinge-CH2-CH3, wherein VH1 is a first variable heavy domain and CH2-CH3 is a first Fc domain;
(b) a light chain comprising, from N-terminus to C-terminus, a VL1-CL, wherein VL1 is a first variable light domain and CL is a constant light domain, and wherein the VH1 and VL1 form MICA/B antigen binding domains; and
(c) a second monomer comprising, from N-terminus to C-terminus, an anti-NKp46 scFv and a second Fc domain, wherein the scFv is covalently attached to the N-terminus of the second Fc domain using a domain linker.

124.-155. (canceled)

156. A heterodimeric antibody, comprising:
(a) a first monomer comprising, from N-terminus to C-terminus, a VH1-CH1-hinge-CH2-CH3-domain linker-scFv, wherein VH1 is a first variable heavy domain, scFv is an anti-NKp46 scFv, and CH2-CH3 is a first Fc domain;
(b) a second monomer comprising, from N-terminus to C-terminus, a VH2-CH1-hinge-CH2-CH3, wherein CH2-CH3 is a second Fc domain; and
(c) a light chain comprising, from N-terminus to C-terminus, a VL1-CL, wherein VL1 is a first variable light domain and CL is a constant light domain,
wherein the VH1 and the VL1 form a first MICA/B antigen binding domain, the VH2 domain and the VL1 form a second MICA/B antigen binding domain,
and the anti-NKp46 scFv comprises a VH3 domain, a scFv linker, and a VL2 domain.

157.-189. (canceled)
